SEQUENCE LISTING (2533 sequences)

SEQ ID NO: 1 - DNA, 25 bp, Homo sapiens
aacctactagtcttgaacaaactgt

SEQ ID NO: 2 - DNA, 25 bp, Homo sapiens
aactgtcatacgtatgggacctaca

SEQ ID NO: 3 - DNA, 25 bp, Homo sapiens
acacttaatctatatgctttacact

SEQ ID NO: 4 - DNA, 25 bp, Homo sapiens
agagctgatcacaagcacaaatctt

SEQ ID NO: 5 - DNA, 25 bp, Homo sapiens
atatgctttacactagctttctgca

SEQ ID NO: 6 - DNA, 25 bp, Homo sapiens
ctttcccactagccatttaataagt

SEQ ID NO: 7 - DNA, 25 bp, Homo sapiens
gctttacactagctttctgcattta

SEQ ID NO: 8 - DNA, 25 bp, Homo sapiens
gctttctgcatttaataggttagaa

SEQ ID NO: 9 - DNA, 25 bp, Homo sapiens
ggacctacacttaatctatatgctt

SEQ ID NO: 10 - DNA, 25 bp, Homo sapiens
gtatgggacctacacttaatctata

SEQ ID NO: 11 - DNA, 25 bp, Homo sapiens
tgatcacaagcacaaatctttccca

SEQ ID NO: 12 - DNA, 25 bp, Homo sapiens
aagctgtgtctgagatctggatctg

SEQ ID NO: 13 - DNA, 25 bp, Homo sapiens
cttgtcctccggtatgttctaaagc

SEQ ID NO: 14 - DNA, 25 bp, Homo sapiens
gaatgctgtcttgacatctcttgcc

SEQ ID NO: 15 - DNA, 25 bp, Homo sapiens
gactggtgttaaatgttgtctacag

SEQ ID NO: 16 - DNA, 25 bp, Homo sapiens
gaggcatcacatgctggtgctgtgt

SEQ ID NO: 17 - DNA, 25 bp, Homo sapiens
gatcttgtattcagtcaggttaaaa

SEQ ID NO: 18 - DNA, 25 bp, Homo sapiens
ggtgatgggttgtgttatgcttgta

SEQ ID NO: 19 - DNA, 25 bp, Homo sapiens
ggtgctgtgtctttatgaatgtttt

SEQ ID NO: 20 - DNA, 25 bp, Homo sapiens
gttatgcttgtattgaatgctgtct

SEQ ID NO: 21 - DNA, 25 bp, Homo sapiens
tccggtatgttctaaagctgtgtct

SEQ ID NO: 22 - DNA, 25 bp, Homo sapiens
tctgagatctggatctgcccatcac

SEQ ID NO: 23 - DNA, 25 bp, Homo sapiens
acaacacctaatgccaccaaagaga

SEQ ID NO: 24 - DNA, 25 bp, Homo sapiens
agaggtgaaggctgagacccgggct

SEQ ID NO: 25 - DNA, 25 bp, Homo sapiens
agcctatggagggcctgggctttct

SEQ ID NO: 26 - DNA, 25 bp, Homo sapiens
agcgactggatggctgtcatccgct

SEQ ID NO: 27 - DNA, 25 bp, Homo sapiens
ccaagttccgttccactggactaga

SEQ ID NO: 28 - DNA, 25 bp, Homo sapiens
ccttcctgagcgacctttgacagag

SEQ ID NO: 29 - DNA, 25 bp, Homo sapiens
gaagagctccggaaattggcctcag

SEQ ID NO: 30 - DNA, 25 bp, Homo sapiens
gaatgccagcacagtggtggtttct

SEQ ID NO: 31 - DNA, 25 bp, Homo sapiens
gcaacgtagctgctccaggagatgc

SEQ ID NO: 32 - DNA, 25 bp, Homo sapiens
gtcatccgctctcagagcagtaccc

SEQ ID NO: 33 - DNA, 25 bp, Homo sapiens
tagagctggagacaccatccttggt

SEQ ID NO: 34 - DNA, 25 bp, Homo sapiens
aaaggctggggtgggtgacttgact

SEQ ID NO: 35 - DNA, 25 bp, Homo sapiens
aacctcactgttcagatgggctgta

SEQ ID NO: 36 - DNA, 25 bp, Homo sapiens
aatatgccccgttgacagtgtttaa

SEQ ID NO: 37 - DNA, 25 bp, Homo sapiens
ataaatatctttcccaatatgcccc

SEQ ID NO: 38 - DNA, 25 bp, Homo sapiens
cactcaaggttcattgggctctgct

SEQ ID NO: 39 - DNA, 25 bp, Homo sapiens
gactaggactgctgatctgcacaat

SEQ ID NO: 40 - DNA, 25 bp, Homo sapiens
gcagggtgcacatgctgcgaggtct

SEQ ID NO: 41 - DNA, 25 bp, Homo sapiens
gcgtgtctgtaaatgtctgcgcagg

SEQ ID NO: 42 - DNA, 25 bp, Homo sapiens
ggagctgtggacagagctccctcac

SEQ ID NO: 43 - DNA, 25 bp, Homo sapiens
gtatgcctgggtacaaacctcactg

SEQ ID NO: 44 - DNA, 25 bp, Homo sapiens
tcctccctgccattacgggagctgt

SEQ ID NO: 45 - DNA, 25 bp, Homo sapiens
aaattgcccttagccgaagagttga

SEQ ID NO: 46 - DNA, 25 bp, Homo sapiens
acggcttctaggtgtacgcactgaa

SEQ ID NO: 47 - DNA, 25 bp, Homo sapiens
atgatctgcaatatgctgctccagg

SEQ ID NO: 48 - DNA, 25 bp, Homo sapiens
caaaaattgaccccactttgtgccg

SEQ ID NO: 49 - DNA, 25 bp, Homo sapiens
cccactttgtgccgggctgacagaa

SEQ ID NO: 50 - DNA, 25 bp, Homo sapiens
gagttagtgctgtcaaggccgattt

SEQ ID NO: 51 - DNA, 25 bp, Homo sapiens
gcaagtacttggtgcagtcggagct

SEQ ID NO: 52 - DNA, 25 bp, Homo sapiens
gctgctccaggcggtcttattggag

SEQ ID NO: 53 - DNA, 25 bp, Homo sapiens
ggtgaacataggatccctgtcaaca

SEQ ID NO: 54 - DNA, 25 bp, Homo sapiens
gtcggagctttacctgagatattca

SEQ ID NO: 55 - DNA, 25 bp, Homo sapiens
tatttcctgcttagacggcttctag

SEQ ID NO: 56 - DNA, 25 bp, Homo sapiens
aattggccttggggactactcggct

SEQ ID NO: 57 - DNA, 25 bp, Homo sapiens
acagaaatgtggctccagttgctct

SEQ ID NO: 58 - DNA, 25 bp, Homo sapiens
cccacctgcccatgtgatgaagcag

SEQ ID NO: 59 - DNA, 25 bp, Homo sapiens
cccacgggactcagaagtgcgccgc

SEQ ID NO: 60 - DNA, 25 bp, Homo sapiens
ctcagctcccacgggactcagaagt

SEQ ID NO: 61 - DNA, 25 bp, Homo sapiens
cttggatcttgagggtctggcacat

SEQ ID NO: 62 - DNA, 25 bp, Homo sapiens
gccgttgccatggtggacggactcc

SEQ ID NO: 63 - DNA, 25 bp, Homo sapiens
ggaaagcccaacgaccatggagaga

SEQ ID NO: 64 - DNA, 25 bp, Homo sapiens
gtcagccgcagactttggaaagccc

SEQ ID NO: 65 - DNA, 25 bp, Homo sapiens
tggagagatgggccgttgccatggt

SEQ ID NO: 66 - DNA, 25 bp, Homo sapiens
tggcacatccttaatcctgtgcccc

SEQ ID NO: 67 - DNA, 25 bp, Homo sapiens
aaatgtggctagtccaaattcaaat

SEQ ID NO: 68 - DNA, 25 bp, Homo sapiens
aatggactagacctgtactaatata

SEQ ID NO: 69 - DNA, 25 bp, Homo sapiens
cactagcaacctgttgagcacttga

SEQ ID NO: 70 - DNA, 25 bp, Homo sapiens
ccggctctcacttcatatgtttaaa

SEQ ID NO: 71 - DNA, 25 bp, Homo sapiens
cctcagacttccgagtggctgggat

SEQ ID NO: 72 - DNA, 25 bp, Homo sapiens
cgccaccacaccaggttgatttttg

SEQ ID NO: 73 - DNA, 25 bp, Homo sapiens
gaaattgagttattgagcactgaaa

SEQ ID NO: 74 - DNA, 25 bp, Homo sapiens
gcaattactactgctaaatgtggga

SEQ ID NO: 75 - DNA, 25 bp, Homo sapiens
ggtagtcactagcaacctgttgagc

SEQ ID NO: 76 - DNA, 25 bp, Homo sapiens
gttaagtatctcaatttttcatatt

SEQ ID NO: 77 - DNA, 25 bp, Homo sapiens
tatatgtagctcacgtatttctatt

SEQ ID NO: 78 - DNA, 25 bp, Homo sapiens
acttctcagggctggaagtcccgtc

SEQ ID NO: 79 - DNA, 25 bp, Homo sapiens
atcttcagtggtggctactgttctc

SEQ ID NO: 80 - DNA, 25 bp, Homo sapiens
caggtgtgtgatggcggctgcaatc

SEQ ID NO: 81 - DNA, 25 bp, Homo sapiens
ctagctgttctacaaaactggagca

SEQ ID NO: 82 - DNA, 25 bp, Homo sapiens
gaggctacttctcagggctggaagt

SEQ ID NO: 83 - DNA, 25 bp, Homo sapiens
gcaatctgtcttgtgggtattaatg

SEQ ID NO: 84 - DNA, 25 bp, Homo sapiens
gctgcaatctgtcttgtgggtatta

SEQ ID NO: 85 - DNA, 25 bp, Homo sapiens
gtcttgtgggtattaatgcaatctt

SEQ ID NO: 86 - DNA, 25 bp, Homo sapiens
tctcagggctggaagtcccgtcagt

SEQ ID NO: 87 - DNA, 25 bp, Homo sapiens
tctctagctgttctacaaaactgga

SEQ ID NO: 88 - DNA, 25 bp, Homo sapiens
tgcaatcttcagtggtggctactgt

SEQ ID NO: 89 - DNA, 25 bp, Homo sapiens
aactcctcatttagatgggcatcat

SEQ ID NO: 90 - DNA, 25 bp, Homo sapiens
aatttctcttgtcaatggccaacag

SEQ ID NO: 91 - DNA, 25 bp, Homo sapiens
cagatgcagctagcaaaccgtttgc

SEQ ID NO: 92 - DNA, 25 bp, Homo sapiens
cataacaacgaaaccaactcctcat

SEQ ID NO: 93 - DNA, 25 bp, Homo sapiens
cctctgattccgaaagtgctactga

SEQ ID NO: 94 - DNA, 25 bp, Homo sapiens
gagttgtctctttcacagagttgtc

SEQ ID NO: 95 - DNA, 25 bp, Homo sapiens
gatacaaatggttcacagttcttca

SEQ ID NO: 96 - DNA, 25 bp, Homo sapiens
gcgagaactttcgttgtctttgtac

SEQ ID NO: 97 - DNA, 25 bp, Homo sapiens
gcggaggtacggatactcagttgtg

SEQ ID NO: 98 - DNA, 25 bp, Homo sapiens
gtgtgccccaaaacatgcgagaact

SEQ ID NO: 99 - DNA, 25 bp, Homo sapiens
gttgtggagagctgattcccaaatc

SEQ ID NO: 100 - DNA, 25 bp, Homo sapiens
acgtttcctgtattctaatctattt

SEQ ID NO: 101 - DNA, 25 bp, Homo sapiens
atcttccaacttccaatatttatcc

SEQ ID NO: 102 - DNA, 25 bp, Homo sapiens
cccgagtctcttacactttattgtg

SEQ ID NO: 103 - DNA, 25 bp, Homo sapiens
gaggtgggacgaatgcacttgcttc

SEQ ID NO: 104 - DNA, 25 bp, Homo sapiens
gatgtccacgtttttgtgactcttc

SEQ ID NO: 105 - DNA, 25 bp, Homo sapiens
ggttacctcagtattacagccaata

SEQ ID NO: 106 - DNA, 25 bp, Homo sapiens
gtggacccacagattgcatctttaa

SEQ ID NO: 107 - DNA, 25 bp, Homo sapiens
tatagtccaagggaccatttctccc

SEQ ID NO: 108 - DNA, 25 bp, Homo sapiens
tgcacttgcttcctgtggcaataaa

SEQ ID NO: 109 - DNA, 25 bp, Homo sapiens
ttatgtttctagtctttcaagctta

SEQ ID NO: 110 - DNA, 25 bp, Homo sapiens
tttatccattcgttgtggacccaca

SEQ ID NO: 111 - DNA, 25 bp, Homo sapiens
aactatttcttggcgacctttgaga

SEQ ID NO: 112 - DNA, 25 bp, Homo sapiens
aattagatttgtctctgggaatgtg

SEQ ID NO: 113 - DNA, 25 bp, Homo sapiens
ctttcaccaaaactatttcttggcg

SEQ ID NO: 114 - DNA, 25 bp, Homo sapiens
ggagctcccatgttgaatttgtttg

SEQ ID NO: 115 - DNA, 25 bp, Homo sapiens
gtgtttctctcctgaggcaaagccc

SEQ ID NO: 116 - DNA, 25 bp, Homo sapiens
gtgtttgtaacataccaacctactg

SEQ ID NO: 117 - DNA, 25 bp, Homo sapiens
tcttggcgacctttgagagatttca

SEQ ID NO: 118 - DNA, 25 bp, Homo sapiens
tgtaacataccaacctactgcagac

SEQ ID NO: 119 - DNA, 25 bp, Homo sapiens
ttgtccacttctccagcaaattaga

SEQ ID NO: 120 - DNA, 25 bp, Homo sapiens
ttgtctctgggaatgtgtttgtaac

SEQ ID NO: 121 - DNA, 25 bp, Homo sapiens
ttttgtccacttctccagcaaatta

SEQ ID NO: 122 - DNA, 25 bp, Homo sapiens
aagctcacatctaaacagcctgtag

SEQ ID NO: 123 - DNA, 25 bp, Homo sapiens
aattccgcaaacactacgactagag

SEQ ID NO: 124 - DNA, 25 bp, Homo sapiens
actgtacctcagttcattgccagag

SEQ ID NO: 125 - DNA, 25 bp, Homo sapiens
caagaacaaactcgtagggactcca

SEQ ID NO: 126 - DNA, 25 bp, Homo sapiens
cgcttcgatcctgaaattccgcaaa

SEQ ID NO: 127 - DNA, 25 bp, Homo sapiens
gaatgctttgaatggcatccgcttc

SEQ ID NO: 128 - DNA, 25 bp, Homo sapiens
gccatatgagctcacagtgcctgca

SEQ ID NO: 129 - DNA, 25 bp, Homo sapiens
gtcagttttgacagtcgctcagaag

SEQ ID NO: 130 - DNA, 25 bp, Homo sapiens
tagccctgaagtgtgggccccgtac

SEQ ID NO: 131 - DNA, 25 bp, Homo sapiens
tctgtacccagcggagttagcgcct

SEQ ID NO: 132 - DNA, 25 bp, Homo sapiens
tttaccccagtagccctgaagtgtg

SEQ ID NO: 133 - DNA, 25 bp, Homo sapiens
aactgcaagcagcctctcagctgat

SEQ ID NO: 134 - DNA, 25 bp, Homo sapiens
caccaggcaccgtgggtcctggatg

SEQ ID NO: 135 - DNA, 25 bp, Homo sapiens
cattcccctttctagctttaactag

SEQ ID NO: 136 - DNA, 25 bp, Homo sapiens
gatgagaggcttcctcagtccagtc

SEQ ID NO: 137 - DNA, 25 bp, Homo sapiens
ggaagattagacactgtggccgagg

SEQ ID NO: 138 - DNA, 25 bp, Homo sapiens
ggacttcatcgtactcgggattttc

SEQ ID NO: 139 - DNA, 25 bp, Homo sapiens
ggccgagggcacgtctagaatcgag

SEQ ID NO: 140 - DNA, 25 bp, Homo sapiens
gggtcctggatggggaactgcaagc

SEQ ID NO: 141 - DNA, 25 bp, Homo sapiens
gtcctcaggtacaaaatccgggcag

SEQ ID NO: 142 - DNA, 25 bp, Homo sapiens
tactcgggattttcttcatctccct

SEQ ID NO: 143 - DNA, 25 bp, Homo sapiens
tagaaccgcgttgggtttgtgggtg

SEQ ID NO: 144 - DNA, 25 bp, Homo sapiens
aagactggtcagcctgcattagtat

SEQ ID NO: 145 - DNA, 25 bp, Homo sapiens
agaattgctgctatactggtggtat

SEQ ID NO: 146 - DNA, 25 bp, Homo sapiens
atatttcacatttatccacacagta

SEQ ID NO: 147 - DNA, 25 bp, Homo sapiens
atttctttgtggtacctgcagttta

SEQ ID NO: 148 - DNA, 25 bp, Homo sapiens
caaaaagatattaatccctctactc

SEQ ID NO: 149 - DNA, 25 bp, Homo sapiens
gagcatattggtatctggatgttcc

SEQ ID NO: 150 - DNA, 25 bp, Homo sapiens
gagtttcctgtagtgctgtttcatt

SEQ ID NO: 151 - DNA, 25 bp, Homo sapiens
ggtggtatggattatcatggcattg

SEQ ID NO: 152 - DNA, 25 bp, Homo sapiens
gtaatgcagatccaatttctttgtg

SEQ ID NO: 153 - DNA, 25 bp, Homo sapiens
gtaggggggctgttagaattgctgc

SEQ ID NO: 154 - DNA, 25 bp, Homo sapiens
tactcccaggttccctttatatgtt

SEQ ID NO: 155 - DNA, 25 bp, Homo sapiens
atctgtgtacaattgtttttgcttc

SEQ ID NO: 156 - DNA, 25 bp, Homo sapiens
atgaatgccttctgcatgttgtaca

SEQ ID NO: 157 - DNA, 25 bp, Homo sapiens
cttgtataatacactactgctgaga

SEQ ID NO: 158 - DNA, 25 bp, Homo sapiens
gaatggatgtgttcgtgcatatata

SEQ ID NO: 159 - DNA, 25 bp, Homo sapiens
gagatggctttcagttgagtttaat

SEQ ID NO: 160 - DNA, 25 bp, Homo sapiens
gcatgttgtacattatctctaacag

SEQ ID NO: 161 - DNA, 25 bp, Homo sapiens
gcatttttggtggtaaatccctttg

SEQ ID NO: 162 - DNA, 25 bp, Homo sapiens
gccacagattcagtagcttttggta

SEQ ID NO: 163 - DNA, 25 bp, Homo sapiens
ggtaaatccctttgccacagattca

SEQ ID NO: 164 - DNA, 25 bp, Homo sapiens
gtagcttttggtaaacttcactgtt

SEQ ID NO: 165 - DNA, 25 bp, Homo sapiens
tgggccaatctggaatagagacatt

SEQ ID NO: 166 - DNA, 25 bp, Homo sapiens
aaagcaaatgatttccatattcctg

SEQ ID NO: 167 - DNA, 25 bp, Homo sapiens
aaagctccaagctgcagtggattta

SEQ ID NO: 168 - DNA, 25 bp, Homo sapiens
aacaacgacaaaaagctccaagctg

SEQ ID NO: 169 - DNA, 25 bp, Homo sapiens
aactggtccttagtcatttgtataa

SEQ ID NO: 170 - DNA, 25 bp, Homo sapiens
acaagtttcttgttcatattgtgaa

SEQ ID NO: 171 - DNA, 25 bp, Homo sapiens
actacctcatactttccttggaaga

SEQ ID NO: 172 - DNA, 25 bp, Homo sapiens
atttccatattcctgattgatcttt

SEQ ID NO: 173 - DNA, 25 bp, Homo sapiens
atttgtatagccttctagaatcaga

SEQ ID NO: 174 - DNA, 25 bp, Homo sapiens
gaaataacctttttgcatattcttt

SEQ ID NO: 175 - DNA, 25 bp, Homo sapiens
gatttgttaaactggtccttagtca

SEQ ID NO: 176 - DNA, 25 bp, Homo sapiens
ggctaaaactacctcatactttcct

SEQ ID NO: 177 - DNA, 25 bp, Homo sapiens
aatgggacattagttcaagtagcaa

SEQ ID NO: 178 - DNA, 25 bp, Homo sapiens
acctgaaatggatgcccctttctgg

SEQ ID NO: 179 - DNA, 25 bp, Homo sapiens
acttggcaactgtacatttccccat

SEQ ID NO: 180 - DNA, 25 bp, Homo sapiens
atctccgacctgaaatggatgcccc

SEQ ID NO: 181 - DNA, 25 bp, Homo sapiens
atgcccctttctggtgtaatcaagg

SEQ ID NO: 182 - DNA, 25 bp, Homo sapiens
ggattcagaagtacattaactggca

SEQ ID NO: 183 - DNA, 25 bp, Homo sapiens
taactggcaagaactacacaatgga

SEQ ID NO: 184 - DNA, 25 bp, Homo sapiens
tatgcatgatgccattggattcaga

SEQ ID NO: 185 - DNA, 25 bp, Homo sapiens
tgcttttttgagggaattgatgatg

SEQ ID NO: 186 - DNA, 25 bp, Homo sapiens
tggtatgaacttttccaacttggca

SEQ ID NO: 187 - DNA, 25 bp, Homo sapiens
ttctggtgtaatcaaggcgctgcct

SEQ ID NO: 188 - DNA, 25 bp, Homo sapiens
aaaccattgcaggtgccagtgtccc

SEQ ID NO: 189 - DNA, 25 bp, Homo sapiens
agtggagtctgtgactgctctgcat

SEQ ID NO: 190 - DNA, 25 bp, Homo sapiens
ataaaaaaaacatcctgctgcggct

SEQ ID NO: 191 - DNA, 25 bp, Homo sapiens
cagaacactcatgtctacagctggc

SEQ ID NO: 192 - DNA, 25 bp, Homo sapiens
gaaacctgttgtgcagagctcttcc

SEQ ID NO: 193 - DNA, 25 bp, Homo sapiens
gaggccaggccatgtttggggcctt

SEQ ID NO: 194 - DNA, 25 bp, Homo sapiens
gcttgtgtatcctcagaccaaactg

SEQ ID NO: 195 - DNA, 25 bp, Homo sapiens
ggccttgttctgacagcattctggc

SEQ ID NO: 196 - DNA, 25 bp, Homo sapiens
gttagccagatgcttgtgtatcctc

SEQ ID NO: 197 - DNA, 25 bp, Homo sapiens
tccacacaccctggctttgaagtgg

SEQ ID NO: 198 - DNA, 25 bp, Homo sapiens
tggcccccaggaaacctgttgtgca

SEQ ID NO: 199 - DNA, 25 bp, Homo sapiens
atccaggttaactgatgctgccatt

SEQ ID NO: 200 - DNA, 25 bp, Homo sapiens
ccgtgtgccccagggggatcaggga

SEQ ID NO: 201 - DNA, 25 bp, Homo sapiens
ctggttggcatttccccattatgta

SEQ ID NO: 202 - DNA, 25 bp, Homo sapiens
gaacatggcttcatccaggttaact

SEQ ID NO: 203 - DNA, 25 bp, Homo sapiens
gctttgctctctctaggtgggcaag

SEQ ID NO: 204 - DNA, 25 bp, Homo sapiens
ggatgcctgtagtagggaactctgg

SEQ ID NO: 205 - DNA, 25 bp, Homo sapiens
gtgagggagccatgctgctgaattc

SEQ ID NO: 206 - DNA, 25 bp, Homo sapiens
gtgggagtgtgaacggatcgctgaa

SEQ ID NO: 207 - DNA, 25 bp, Homo sapiens
gtgttgggtagggcagactctgctt

SEQ ID NO: 208 - DNA, 25 bp, Homo sapiens
tcgcccatctgttgctgtgggagtg

SEQ ID NO: 209 - DNA, 25 bp, Homo sapiens
tgggctgaggtgggattttccctcc

SEQ ID NO: 210 - DNA, 25 bp, Homo sapiens
atggcatccacgcatgggatctgca

SEQ ID NO: 211 - DNA, 25 bp, Homo sapiens
atgggatctgcaagctggagccctc

SEQ ID NO: 212 - DNA, 25 bp, Homo sapiens
catctctgcactaactcatctgaat

SEQ ID NO: 213 - DNA, 25 bp, Homo sapiens
cggcagtggctgtaaggtcaccttc

SEQ ID NO: 214 - DNA, 25 bp, Homo sapiens
ctgtgactgggccagggcacacgtt

SEQ ID NO: 215 - DNA, 25 bp, Homo sapiens
gacagactgggctgaggctgacagg

SEQ ID NO: 216 - DNA, 25 bp, Homo sapiens
ggctgcaggcagtctactggcagga

SEQ ID NO: 217 - DNA, 25 bp, Homo sapiens
ggtggcagtcttggtcagtagttta

SEQ ID NO: 218 - DNA, 25 bp, Homo sapiens
ggtgtagaccagccctgggatttcc

SEQ ID NO: 219 - DNA, 25 bp, Homo sapiens
tcagtgctgatgccatgccaactgc

SEQ ID NO: 220 - DNA, 25 bp, Homo sapiens
tctgcacacgcaggttctgggcgac

SEQ ID NO: 221 - DNA, 25 bp, Homo sapiens
cctgcacactgggctattgctttat

SEQ ID NO: 222 - DNA, 25 bp, Homo sapiens
ctccacatgctgcaaggacagactg

SEQ ID NO: 223 - DNA, 25 bp, Homo sapiens
ctgggctattgctttatccctatcc

SEQ ID NO: 224 - DNA, 25 bp, Homo sapiens
gaaaggtagggatgggccagcctcc

SEQ ID NO: 225 - DNA, 25 bp, Homo sapiens
gaagggctgtgagcaggtgtaaggg

SEQ ID NO: 226 - DNA, 25 bp, Homo sapiens
gacagtaggcaggctgagtggccca

SEQ ID NO: 227 - DNA, 25 bp, Homo sapiens
gagcaggtgtaagggctcccacatc

SEQ ID NO: 228 - DNA, 25 bp, Homo sapiens
gctttatccctatcctgagagcagc

SEQ ID NO: 229 - DNA, 25 bp, Homo sapiens
tcagctgttgggagacagtaggcag

SEQ ID NO: 230 - DNA, 25 bp, Homo sapiens
tgctccagcctgcaacttagtggaa

SEQ ID NO: 231 - DNA, 25 bp, Homo sapiens
ttagtggaaggaattacttcctcct

SEQ ID NO: 232 - DNA, 25 bp, Homo sapiens
aacagattcatcattattcctaaag

SEQ ID NO: 233 - DNA, 25 bp, Homo sapiens
agtgcctacttttcttcgatatcat

SEQ ID NO: 234 - DNA, 25 bp, Homo sapiens
gaacattgtcatttagccaagcaaa

SEQ ID NO: 235 - DNA, 25 bp, Homo sapiens
gagatttctcatatgtttgcgtata

SEQ ID NO: 236 - DNA, 25 bp, Homo sapiens
gagccagcaggttcaccagaaagct

SEQ ID NO: 237 - DNA, 25 bp, Homo sapiens
gagcgtttgctggaacacattatgc

SEQ ID NO: 238 - DNA, 25 bp, Homo sapiens
gatatcattagctgtttttcgaaac

SEQ ID NO: 239 - DNA, 25 bp, Homo sapiens
ggagccagtcgaagatcctgttcaa

SEQ ID NO: 240 - DNA, 25 bp, Homo sapiens
ggcaggcatttcttgaacattgtca

SEQ ID NO: 241 - DNA, 25 bp, Homo sapiens
tagaaagtatccaccagtgcctact

SEQ ID NO: 242 - DNA, 25 bp, Homo sapiens
tatgcttctgtggcaggcatttctt

SEQ ID NO: 243 - DNA, 25 bp, Homo sapiens
acagcccctgcacaaggctgacaca

SEQ ID NO: 244 - DNA, 25 bp, Homo sapiens
actaatgctatcaaagtcctccttt

SEQ ID NO: 245 - DNA, 25 bp, Homo sapiens
agcccggctgctctagcaggaatgt

SEQ ID NO: 246 - DNA, 25 bp, Homo sapiens
aggactctgcttgtttcagtagccc

SEQ ID NO: 247 - DNA, 25 bp, Homo sapiens
ccttgactggtgggctttttacgtg

SEQ ID NO: 248 - DNA, 25 bp, Homo sapiens
gcttctcccacgggtagtgtcagtt

SEQ ID NO: 249 - DNA, 25 bp, Homo sapiens
ggacctctccctagtgattatctag

SEQ ID NO: 250 - DNA, 25 bp, Homo sapiens
taagacaccttttataagcctccct

SEQ ID NO: 251 - DNA, 25 bp, Homo sapiens
tacatttgcggtttggccacaggtc

SEQ ID NO: 252 - DNA, 25 bp, Homo sapiens
ttaaaaagtcacttcagccccacaa

SEQ ID NO: 253 - DNA, 25 bp, Homo sapiens
ttatctagccagctacaccttactc

SEQ ID NO: 254 - DNA, 25 bp, Homo sapiens
aagtgtatattgacatttctggaat

SEQ ID NO: 255 - DNA, 25 bp, Homo sapiens
cactcacaagagtgtataccctgtg

SEQ ID NO: 256 - DNA, 25 bp, Homo sapiens
gcacaatttgggccactcacaagag

SEQ ID NO: 257 - DNA, 25 bp, Homo sapiens
ggaaatgtattaattgcccaaagta

SEQ ID NO: 258 - DNA, 25 bp, Homo sapiens
ggcaggagagccgaggtaagactta

SEQ ID NO: 259 - DNA, 25 bp, Homo sapiens
ggtaagacttactgtaggctgtcgt

SEQ ID NO: 260 - DNA, 25 bp, Homo sapiens
gttttttgtctttgcgatggagtct

SEQ ID NO: 261 - DNA, 25 bp, Homo sapiens
taaacagttacctacattctcctct

SEQ ID NO: 262 - DNA, 25 bp, Homo sapiens
tactgtaggctgtcgttttttttgt

SEQ ID NO: 263 - DNA, 25 bp, Homo sapiens
tcctctgcatgcttgtctttagagg

SEQ ID NO: 264 - DNA, 25 bp, Homo sapiens
tgtttgcacaatttgggccactcac

SEQ ID NO: 265 - DNA, 25 bp, Homo sapiens
aagtcgtagggcagctatggaaacc

SEQ ID NO: 266 - DNA, 25 bp, Homo sapiens
agaagccttcaccttccagcttttg

SEQ ID NO: 267 - DNA, 25 bp, Homo sapiens
agacaagcagtgtgatagagtcctt

SEQ ID NO: 268 - DNA, 25 bp, Homo sapiens
agtcactgtatatacgtgcacattt

SEQ ID NO: 269 - DNA, 25 bp, Homo sapiens
ccagcttttgtctggcctgtgctgc

SEQ ID NO: 270 - DNA, 25 bp, Homo sapiens
cgtgggagccactggtctgtgcaca

SEQ ID NO: 271 - DNA, 25 bp, Homo sapiens
gcctgggatgctccattgcatttgt

SEQ ID NO: 272 - DNA, 25 bp, Homo sapiens
ggcagctatggaaaccactgggttc

SEQ ID NO: 273 - DNA, 25 bp, Homo sapiens
ggtgggtttagtcatctcggaagtc

SEQ ID NO: 274 - DNA, 25 bp, Homo sapiens
gtccttggtgggtttagtcatctcg

SEQ ID NO: 275 - DNA, 25 bp, Homo sapiens
tcacctagtcactgtatatacgtgc

SEQ ID NO: 276 - DNA, 25 bp, Homo sapiens
tcgtagggcagctatggaaaccact

SEQ ID NO: 277 - DNA, 25 bp, Homo sapiens
tctcggaagtcgtagggcagctatg

SEQ ID NO: 278 - DNA, 25 bp, Homo sapiens
tgagtggccaagacaagcagtgtga

SEQ ID NO: 279 - DNA, 25 bp, Homo sapiens
tggtctgtgcacatccacggtgggt

SEQ ID NO: 280 - DNA, 25 bp, Homo sapiens
ttcatcccagcctgggatgctccat

SEQ ID NO: 281 - DNA, 25 bp, Homo sapiens
aaagactgctgcttctggaattccc

SEQ ID NO: 282 - DNA, 25 bp, Homo sapiens
aagaagctgtctgcgaagtggccct

SEQ ID NO: 283 - DNA, 25 bp, Homo sapiens
aagcaggtcctggcacaatgtttat

SEQ ID NO: 284 - DNA, 25 bp, Homo sapiens
acacatggatccaggctatctcttc

SEQ ID NO: 285 - DNA, 25 bp, Homo sapiens
agagaagcggttcagcctttttggc

SEQ ID NO: 286 - DNA, 25 bp, Homo sapiens
agcgaggtccctgtgagtttgaaag

SEQ ID NO: 287 - DNA, 25 bp, Homo sapiens
ccttctcttaccttttcagtgaaat

SEQ ID NO: 288 - DNA, 25 bp, Homo sapiens
cgccatctcctctgataaacacgag

SEQ ID NO: 289 - DNA, 25 bp, Homo sapiens
ctgtgcctaatgttcctcaatgtgg

SEQ ID NO: 290 - DNA, 25 bp, Homo sapiens
gaaccaacacattactctctgtgcc

SEQ ID NO: 291 - DNA, 25 bp, Homo sapiens
ggcaatgagtacctcttccaagcca

SEQ ID NO: 292 - DNA, 25 bp, Homo sapiens
aagcagtatctgttatttagctgta

SEQ ID NO: 293 - DNA, 25 bp, Homo sapiens
aatacttccctcaattctgtaaatt

SEQ ID NO: 294 - DNA, 25 bp, Homo sapiens
aatttagtgatcaaactgccattca

SEQ ID NO: 295 - DNA, 25 bp, Homo sapiens
atgactttatacccaattctacata

SEQ ID NO: 296 - DNA, 25 bp, Homo sapiens
gatctatctttttttgttaccttca

SEQ ID NO: 297 - DNA, 25 bp, Homo sapiens
gccattcacagtgtaaggcagcact

SEQ ID NO: 298 - DNA, 25 bp, Homo sapiens
ggcagcacttaaatttcgaacctaa

SEQ ID NO: 299 - DNA, 25 bp, Homo sapiens
ttaccttcagatgttcactaaataa

SEQ ID NO: 300 - DNA, 25 bp, Homo sapiens
ttgaccccaaatgactttataccca

SEQ ID NO: 301 - DNA, 25 bp, Homo sapiens
ttgggatttttggtgcttatatgct

SEQ ID NO: 302 - DNA, 25 bp, Homo sapiens
tttggagtactgtttcttccttcaa

SEQ ID NO: 303 - DNA, 25 bp, Homo sapiens
acccatttgtgcattgagttttctt

SEQ ID NO: 304 - DNA, 25 bp, Homo sapiens
agcacttttatactaattaacccat

SEQ ID NO: 305 - DNA, 25 bp, Homo sapiens
gagcagtcagcattgcacctgctat

SEQ ID NO: 306 - DNA, 25 bp, Homo sapiens
gatacccagtatgcttaacgtgaaa

SEQ ID NO: 307 - DNA, 25 bp, Homo sapiens
gatggcagttcttatctgcatcact

SEQ ID NO: 308 - DNA, 25 bp, Homo sapiens
gcattgcacctgctatggagaaggg

SEQ ID NO: 309 - DNA, 25 bp, Homo sapiens
gctcactggccagagacattgatgg

SEQ ID NO: 310 - DNA, 25 bp, Homo sapiens
ggaagtttgttgtagtatgcctcaa

SEQ ID NO: 311 - DNA, 25 bp, Homo sapiens
gtaaatacttggacagaggttgctg

SEQ ID NO: 312 - DNA, 25 bp, Homo sapiens
gttttcaatttgctcactggccaga

SEQ ID NO: 313 - DNA, 25 bp, Homo sapiens
tggtaacttttcaaacagcccttag

SEQ ID NO: 314 - DNA, 25 bp, Homo sapiens
cagaagacccctgactcatcatttg

SEQ ID NO: 315 - DNA, 25 bp, Homo sapiens
cagtcccttataattggtgcatagc

SEQ ID NO: 316 - DNA, 25 bp, Homo sapiens
cattccctctcatctcaggtagaag

SEQ ID NO: 317 - DNA, 25 bp, Homo sapiens
cctcctccagggtgattttatgatc

SEQ ID NO: 318 - DNA, 25 bp, Homo sapiens
ctcatcatttgtggcagtcccttat

SEQ ID NO: 319 - DNA, 25 bp, Homo sapiens
gatcctggtttcataacttcctgta

SEQ ID NO: 320 - DNA, 25 bp, Homo sapiens
gatggtttccacatttagatcctgg

SEQ ID NO: 321 - DNA, 25 bp, Homo sapiens
tacacactgtcatgcttcatcattc

SEQ ID NO: 322 - DNA, 25 bp, Homo sapiens
tgatcagtgttgttgctctaggaag

SEQ ID NO: 323 - DNA, 25 bp, Homo sapiens
tgtcctaattcttctgtcctgagaa

SEQ ID NO: 324 - DNA, 25 bp, Homo sapiens
ttttccgtttgcttttgttccaatg

SEQ ID NO: 325 - DNA, 25 bp, Homo sapiens
aagccttacagttatcctgcaaggg

SEQ ID NO: 326 - DNA, 25 bp, Homo sapiens
atagtcccaccttggagcatttatg

SEQ ID NO: 327 - DNA, 25 bp, Homo sapiens
atcagctgttgcaggcagtgtctta

SEQ ID NO: 328 - DNA, 25 bp, Homo sapiens
caccttatacatcacttcctgtttt

SEQ ID NO: 329 - DNA, 25 bp, Homo sapiens
catcaagcatcattgtccccatgca

SEQ ID NO: 330 - DNA, 25 bp, Homo sapiens
cgcctaggatttcagccatgcgcgc

SEQ ID NO: 331 - DNA, 25 bp, Homo sapiens
gaagcctaattgtcacatcaagcat

SEQ ID NO: 332 - DNA, 25 bp, Homo sapiens
gagcaaagcatcggtcatgtgtgta

SEQ ID NO: 333 - DNA, 25 bp, Homo sapiens
gcatgtctaattcatttactcacca

SEQ ID NO: 334 - DNA, 25 bp, Homo sapiens
gtgtattttttcatagtcccacctt

SEQ ID NO: 335 - DNA, 25 bp, Homo sapiens
tctttcttctcgcctaggatttcag

SEQ ID NO: 336 - DNA, 25 bp, Homo sapiens
aacctacagcatattcttcacgcag

SEQ ID NO: 337 - DNA, 25 bp, Homo sapiens
aagattggccatgttccacttggaa

SEQ ID NO: 338 - DNA, 25 bp, Homo sapiens
acaattcttagatctggtgtccagc

SEQ ID NO: 339 - DNA, 25 bp, Homo sapiens
acagatgccaattacggtgtacagt

SEQ ID NO: 340 - DNA, 25 bp, Homo sapiens
gaattccagatgtaggcattccccc

SEQ ID NO: 341 - DNA, 25 bp, Homo sapiens
gagaagatcctgtcacaattcttag

SEQ ID NO: 342 - DNA, 25 bp, Homo sapiens
gagtgcagctaacatgagtatcatc

SEQ ID NO: 343 - DNA, 25 bp, Homo sapiens
gagtttggtccctaaatttgcatga

SEQ ID NO: 344 - DNA, 25 bp, Homo sapiens
gcgtaactccatctgacaaattcaa

SEQ ID NO: 345 - DNA, 25 bp, Homo sapiens
tagagaaacctgcgtaactccatct

SEQ ID NO: 346 - DNA, 25 bp, Homo sapiens
tgcttcaggagtttcatgttggatc

SEQ ID NO: 347 - DNA, 25 bp, Homo sapiens
agttcctggaatggcacgttgctgc

SEQ ID NO: 348 - DNA, 25 bp, Homo sapiens
attttaagccctatcactgacacat

SEQ ID NO: 349 - DNA, 25 bp, Homo sapiens
cactgacacatcagcatgttttctg

SEQ ID NO: 350 - DNA, 25 bp, Homo sapiens
ctgccacaaaaatgttcacttcgaa

SEQ ID NO: 351 - DNA, 25 bp, Homo sapiens
gaatggcacgttgctgccagtgccc

SEQ ID NO: 352 - DNA, 25 bp, Homo sapiens
gatgacgaatcctgctctaaaatac

SEQ ID NO: 353 - DNA, 25 bp, Homo sapiens
ggcccgcacgttttatgaggttgat

SEQ ID NO: 354 - DNA, 25 bp, Homo sapiens
ggcttgtgatgacgaatcctgctct

SEQ ID NO: 355 - DNA, 25 bp, Homo sapiens
gtcagttaacgtcacccaaaagcac

SEQ ID NO: 356 - DNA, 25 bp, Homo sapiens
tatcggtgctatgtgtttggtttat

SEQ ID NO: 357 - DNA, 25 bp, Homo sapiens
ttatgacagtatcgaggcttgtgat

SEQ ID NO: 358 - DNA, 25 bp, Homo sapiens
agtccaaacctttatctgtctgtta

SEQ ID NO: 359 - DNA, 25 bp, Homo sapiens
caacaccacaaagatcgcatctgtt

SEQ ID NO: 360 - DNA, 25 bp, Homo sapiens
caaggcatgggaccaggcctgcttg

SEQ ID NO: 361 - DNA, 25 bp, Homo sapiens
ccactggcaaggccaaggtctcctc

SEQ ID NO: 362 - DNA, 25 bp, Homo sapiens
gagcaaagccttatccgaatcggat

SEQ ID NO: 363 - DNA, 25 bp, Homo sapiens
ggatttagcactggggtctcagcac

SEQ ID NO: 364 - DNA, 25 bp, Homo sapiens
ggcctgcttgcctatgtgtgatggc

SEQ ID NO: 365 - DNA, 25 bp, Homo sapiens
gtcaattagagcgatcccaaggcat

SEQ ID NO: 366 - DNA, 25 bp, Homo sapiens
gtctgagactaagtgatctgccctc

SEQ ID NO: 367 - DNA, 25 bp, Homo sapiens
gtctttaattttgagcaccttacca

SEQ ID NO: 368 - DNA, 25 bp, Homo sapiens
tcctccacgttttttctgcaattaa

SEQ ID NO: 369 - DNA, 25 bp, Homo sapiens
aaggtgttcattttgtcacaagctg

SEQ ID NO: 370 - DNA, 25 bp, Homo sapiens
atgagcatctcaaatgttttctgca

SEQ ID NO: 371 - DNA, 25 bp, Homo sapiens
atggccgtatcaaatggtagctgaa

SEQ ID NO: 372 - DNA, 25 bp, Homo sapiens
atgggattttctagtttcctgcctt

SEQ ID NO: 373 - DNA, 25 bp, Homo sapiens
atttgagcactggtctctcttggaa

SEQ ID NO: 374 - DNA, 25 bp, Homo sapiens
ctcgtcaatccatcagcaatgcttc

SEQ ID NO: 375 - DNA, 25 bp, Homo sapiens
gcaatgcttctctcatagtgtcata

SEQ ID NO: 376 - DNA, 25 bp, Homo sapiens
ggaccatccaaatttatggccgtat

SEQ ID NO: 377 - DNA, 25 bp, Homo sapiens
ggacgtagagttggcctttttacag

SEQ ID NO: 378 - DNA, 25 bp, Homo sapiens
tcctgccttcagagtatctaatcct

SEQ ID NO: 379 - DNA, 25 bp, Homo sapiens
ttaatgatctggtggtctcctcgtc

SEQ ID NO: 380 - DNA, 25 bp, Homo sapiens
atgcatattcaacacactgccttat

SEQ ID NO: 381 - DNA, 25 bp, Homo sapiens
ccaagtcctttaactcgttgcagtc

SEQ ID NO: 382 - DNA, 25 bp, Homo sapiens
ccagtccttggctgtatccatgtaa

SEQ ID NO: 383 - DNA, 25 bp, Homo sapiens
gaaatccccgggaagagttagcctg

SEQ ID NO: 384 - DNA, 25 bp, Homo sapiens
gaattgctgtctagccttagtcaat

SEQ ID NO: 385 - DNA, 25 bp, Homo sapiens
gagttagcctggatagccttgaaaa

SEQ ID NO: 386 - DNA, 25 bp, Homo sapiens
gtatcatgtatctctctgtggtggt

SEQ ID NO: 387 - DNA, 25 bp, Homo sapiens
gtggtggttcattccacaggacgaa

SEQ ID NO: 388 - DNA, 25 bp, Homo sapiens
taagtacttggtcccgtggatgctc

SEQ ID NO: 389 - DNA, 25 bp, Homo sapiens
tgaaagttggggcccagtccttggc

SEQ ID NO: 390 - DNA, 25 bp, Homo sapiens
tggatgctctttcaatgcagcaccc

SEQ ID NO: 391 - DNA, 25 bp, Homo sapiens
aaatctccttcaaaatatccaatcc

SEQ ID NO: 392 - DNA, 25 bp, Homo sapiens
aagctgacacctaagtttaccaaca

SEQ ID NO: 393 - DNA, 25 bp, Homo sapiens
acatgctacagctgatggctttccc

SEQ ID NO: 394 - DNA, 25 bp, Homo sapiens
caaggacttctttatccgagcgctg

SEQ ID NO: 395 - DNA, 25 bp, Homo sapiens
ccgagcgctggattgcatgagaaga

SEQ ID NO: 396 - DNA, 25 bp, Homo sapiens
ctggctgcaacgatttgccgcaaac

SEQ ID NO: 397 - DNA, 25 bp, Homo sapiens
gaatggtattcgtttcacctgttgt

SEQ ID NO: 398 - DNA, 25 bp, Homo sapiens
gatgagcaccagttacacaaggact

SEQ ID NO: 399 - DNA, 25 bp, Homo sapiens
gatgcctcctgattatatttcacat

SEQ ID NO: 400 - DNA, 25 bp, Homo sapiens
gtagaaattatgtggctggctgcaa

SEQ ID NO: 401 - DNA, 25 bp, Homo sapiens
gtcagtgacacttgaacaatgctca

SEQ ID NO: 402 - DNA, 25 bp, Homo sapiens
aaatatctgatcttaccctgggaca

SEQ ID NO: 403 - DNA, 25 bp, Homo sapiens
agatgacgcctttagctgatctctg

SEQ ID NO: 404 - DNA, 25 bp, Homo sapiens
atcgtcagctggagccgtacgagct

SEQ ID NO: 405 - DNA, 25 bp, Homo sapiens
gaaactgcagcttctccataattta

SEQ ID NO: 406 - DNA, 25 bp, Homo sapiens
gagggaactggattggacccttcca

SEQ ID NO: 407 - DNA, 25 bp, Homo sapiens
gctgtgacaacactgtggtgcgcat

SEQ ID NO: 408 - DNA, 25 bp, Homo sapiens
ggaattctgtttgtctggtctttga

SEQ ID NO: 409 - DNA, 25 bp, Homo sapiens
gtgcgcatggtctccagtggaaaac

SEQ ID NO: 410 - DNA, 25 bp, Homo sapiens
taaccaacccagtgatttacatgct

SEQ ID NO: 411 - DNA, 25 bp, Homo sapiens
tcataccagtcagtatttcccagcc

SEQ ID NO: 412 - DNA, 25 bp, Homo sapiens
ttcatggcccggcccagatgaaagt

SEQ ID NO: 413 - DNA, 25 bp, Homo sapiens
aaagcccttcatctaatatttgttg

SEQ ID NO: 414 - DNA, 25 bp, Homo sapiens
aaatgcttgccgctttagaggtgga

SEQ ID NO: 415 - DNA, 25 bp, Homo sapiens
aagccaatcatttgtaaccattcta

SEQ ID NO: 416 - DNA, 25 bp, Homo sapiens
accatacactggatgacctagtcga

SEQ ID NO: 417 - DNA, 25 bp, Homo sapiens
gcattcattgacacatagctctaat

SEQ ID NO: 418 - DNA, 25 bp, Homo sapiens
gctagtagaatggcagcacgctgta

SEQ ID NO: 419 - DNA, 25 bp, Homo sapiens
gtaaccattctagcagtgtcatatt

SEQ ID NO: 420 - DNA, 25 bp, Homo sapiens
gtagacacctttcagtaagccaatc

SEQ ID NO: 421 - DNA, 25 bp, Homo sapiens
tatacggtagttgctttagggggtg

SEQ ID NO: 422 - DNA, 25 bp, Homo sapiens
tggtgctcataaaaggccccagtcg

SEQ ID NO: 423 - DNA, 25 bp, Homo sapiens
ttactgtattgtgtactggctataa

SEQ ID NO: 424 - DNA, 25 bp, Homo sapiens
acacactcttactcccgtgatgtgt

SEQ ID NO: 425 - DNA, 25 bp, Homo sapiens
agtcaaaggctgatgtcctgtttct

SEQ ID NO: 426 - DNA, 25 bp, Homo sapiens
atttgaccacgtccattgtttccat

SEQ ID NO: 427 - DNA, 25 bp, Homo sapiens
caagccatggcaatatctgtcccac

SEQ ID NO: 428 - DNA, 25 bp, Homo sapiens
catctacatccatatcatgcccatg

SEQ ID NO: 429 - DNA, 25 bp, Homo sapiens
catgcccatgcatctgtaacttgct

SEQ ID NO: 430 - DNA, 25 bp, Homo sapiens
gagtttgttcaatgcatgtgtctgt

SEQ ID NO: 431 - DNA, 25 bp, Homo sapiens
gtgatgtgtgttaagggctccgatg

SEQ ID NO: 432 - DNA, 25 bp, Homo sapiens
tgaatttctgcacgctgttgtctgt

SEQ ID NO: 433 - DNA, 25 bp, Homo sapiens
tgtaacttgcttttcccgtgtaaga

SEQ ID NO: 434 - DNA, 25 bp, Homo sapiens
tgtttccatcttttgggctgttctt

SEQ ID NO: 435 - DNA, 25 bp, Homo sapiens
aaaaccttccgagtgagctcacatc

SEQ ID NO: 436 - DNA, 25 bp, Homo sapiens
aacatgcagcagttttcagtggaga

SEQ ID NO: 437 - DNA, 25 bp, Homo sapiens
acatcttattcgacactttagaatt

SEQ ID NO: 438 - DNA, 25 bp, Homo sapiens
agagctcaaaccttagtcaacacca

SEQ ID NO: 439 - DNA, 25 bp, Homo sapiens
agctcaaaacttgctaggcatcaga

SEQ ID NO: 440 - DNA, 25 bp, Homo sapiens
gaactcacatcttatcaggcatcag

SEQ ID NO: 441 - DNA, 25 bp, Homo sapiens
gctcagatcttactagacatcggcg

SEQ ID NO: 442 - DNA, 25 bp, Homo sapiens
gctttcaggcacagctcaaaacttg

SEQ ID NO: 443 - DNA, 25 bp, Homo sapiens
gctttgcagagagctcagatcttac

SEQ ID NO: 444 - DNA, 25 bp, Homo sapiens
ggagagcattcaacctgaactcaca

SEQ ID NO: 445 - DNA, 25 bp, Homo sapiens
tagacatcggcgaattcacactggg

SEQ ID NO: 446 - DNA, 25 bp, Homo sapiens
atggcgaagtctttagtctttttca

SEQ ID NO: 447 - DNA, 25 bp, Homo sapiens
atttgcagcatgcttgactttacca

SEQ ID NO: 448 - DNA, 25 bp, Homo sapiens
cactaagaccttgttatggcgaagt

SEQ ID NO: 449 - DNA, 25 bp, Homo sapiens
cggacactattatcactaagacctt

SEQ ID NO: 450 - DNA, 25 bp, Homo sapiens
gactttaccaattctgatgacatct

SEQ ID NO: 451 - DNA, 25 bp, Homo sapiens
gataatctgggaaagacaccaaatc

SEQ ID NO: 452 - DNA, 25 bp, Homo sapiens
gatgacatctttacggacactatta

SEQ ID NO: 453 - DNA, 25 bp, Homo sapiens
gttgtcgcaaaggggataatctggg

SEQ ID NO: 454 - DNA, 25 bp, Homo sapiens
tatgccttacctttgtaaatatttt

SEQ ID NO: 455 - DNA, 25 bp, Homo sapiens
tgcttgtgttgtcgcaaaggggata

SEQ ID NO: 456 - DNA, 25 bp, Homo sapiens
ttagtctttttcatgtattttcctc

SEQ ID NO: 457 - DNA, 25 bp, Homo sapiens
aactatttcttggcgacctttgaga

SEQ ID NO: 458 - DNA, 25 bp, Homo sapiens
aattagatttgtctctgggaatgtg

SEQ ID NO: 459 - DNA, 25 bp, Homo sapiens
ctttcaccaaaactatttcttggcg

SEQ ID NO: 460 - DNA, 25 bp, Homo sapiens
ggagctcccatgttgaatttgtttg

SEQ ID NO: 461 - DNA, 25 bp, Homo sapiens
gtgtttctctcctgaggcaaagccc

SEQ ID NO: 462 - DNA, 25 bp, Homo sapiens
gtgtttgtaacataccaacctactg

SEQ ID NO: 463 - DNA, 25 bp, Homo sapiens
tcttggcgacctttgagagatttca

SEQ ID NO: 464 - DNA, 25 bp, Homo sapiens
tgtaacataccaacctactgcagac

SEQ ID NO: 465 - DNA, 25 bp, Homo sapiens
ttgtccacttctccagcaaattaga

SEQ ID NO: 466 - DNA, 25 bp, Homo sapiens
ttgtctctgggaatgtgtttgtaac

SEQ ID NO: 467 - DNA, 25 bp, Homo sapiens
ttttgtccacttctccagcaaatta

SEQ ID NO: 468 - DNA, 25 bp, Homo sapiens
aatctggtgaacgctacgcttacat

SEQ ID NO: 469 - DNA, 25 bp, Homo sapiens
caagtgcgagacctgggtgtccaac

SEQ ID NO: 470 - DNA, 25 bp, Homo sapiens
gaggagatctaagcagcgttcccac

SEQ ID NO: 471 - DNA, 25 bp, Homo sapiens
gagttccgcagagcttactatacgc

SEQ ID NO: 472 - DNA, 25 bp, Homo sapiens
gtatcgtcttcctcaacaagtgcga

SEQ ID NO: 473 - DNA, 25 bp, Homo sapiens
gttcgctatctcttgtgtcaaatct

SEQ ID NO: 474 - DNA, 25 bp, Homo sapiens
tactatacgcggtctgtcctaatct

SEQ ID NO: 475 - DNA, 25 bp, Homo sapiens
tcgacatgaccaccttcagcaagga

SEQ ID NO: 476 - DNA, 25 bp, Homo sapiens
tgcaaaaacaatcctctttctctct

SEQ ID NO: 477 - DNA, 25 bp, Homo sapiens
tgcgctacaaccacatgctgcggaa

SEQ ID NO: 478 - DNA, 25 bp, Homo sapiens
tgtcctaatctttgtggtgttcgct

SEQ ID NO: 479 - DNA, 25 bp, Homo sapiens
aaagcgccagtgaactctgactgta

SEQ ID NO: 480 - DNA, 25 bp, Homo sapiens
aacaacgcattgccatagctcgtgc

SEQ ID NO: 481 - DNA, 25 bp, Homo sapiens
agccacgtcagctctggatacagaa

SEQ ID NO: 482 - DNA, 25 bp, Homo sapiens
caaaggaactcagctctctggtggc

SEQ ID NO: 483 - DNA, 25 bp, Homo sapiens
cagcctcatattttgcttttggatg

SEQ ID NO: 484 - DNA, 25 bp, Homo sapiens
gagtgagagacatcatcaagtggag

SEQ ID NO: 485 - DNA, 25 bp, Homo sapiens
gcccttgttagacagcctcatattt

SEQ ID NO: 486 - DNA, 25 bp, Homo sapiens
gtcactgcctaataaatatagcact

SEQ ID NO: 487 - DNA, 25 bp, Homo sapiens
tcctcagtcaagttcagagtcttca

SEQ ID NO: 488 - DNA, 25 bp, Homo sapiens
tctgtttaacatttcctcagtcaag

SEQ ID NO: 489 - DNA, 25 bp, Homo sapiens
tttggatgaagccacgtcagctctg

SEQ ID NO: 490 - DNA, 25 bp, Homo sapiens
aagctatgtgtatcttctgtgtaaa

SEQ ID NO: 491 - DNA, 25 bp, Homo sapiens
aatggtgtggctagcatttcccttt

SEQ ID NO: 492 - DNA, 25 bp, Homo sapiens
acttccttggaatatagctgcatta

SEQ ID NO: 493 - DNA, 25 bp, Homo sapiens
agtcactttccttatgtatcatcta

SEQ ID NO: 494 - DNA, 25 bp, Homo sapiens
cttccctaagtcactttccttatgt

SEQ ID NO: 495 - DNA, 25 bp, Homo sapiens
gaagcctgttgggccagaagacaga

SEQ ID NO: 496 - DNA, 25 bp, Homo sapiens
gaagggaacacatttccttctgaac

SEQ ID NO: 497 - DNA, 25 bp, Homo sapiens
gcaatccaggcctctgttgaaaaga

SEQ ID NO: 498 - DNA, 25 bp, Homo sapiens
taagtttgcttttgaccatcacctc

SEQ ID NO: 499 - DNA, 25 bp, Homo sapiens
taatccatttagcaatccaggcctc

SEQ ID NO: 500 - DNA, 25 bp, Homo sapiens
tcacctcccagtagcaatttgcttt

SEQ ID NO: 501 - DNA, 25 bp, Homo sapiens
aaaccatttgtatctggcatcactt

SEQ ID NO: 502 - DNA, 25 bp, Homo sapiens
aattttcatcttactgcacaatcaa

SEQ ID NO: 503 - DNA, 25 bp, Homo sapiens
acatgcggcttttctgcatcaactg

SEQ ID NO: 504 - DNA, 25 bp, Homo sapiens
gaggctgggcctgaacagggaggtg

SEQ ID NO: 505 - DNA, 25 bp, Homo sapiens
gtgctcagtcgtacgacctgtacct

SEQ ID NO: 506 - DNA, 25 bp, Homo sapiens
taacacacgacatgcggcttttctg

SEQ ID NO: 507 - DNA, 25 bp, Homo sapiens
taatttgcttcatttccttgctatt

SEQ ID NO: 508 - DNA, 25 bp, Homo sapiens
taggaatgaactccagaggctgggc

SEQ ID NO: 509 - DNA, 25 bp, Homo sapiens
tctaatggttacttgctcgtgcgtt

SEQ ID NO: 510 - DNA, 25 bp, Homo sapiens
tctggcatcacttactaacacacga

SEQ ID NO: 511 - DNA, 25 bp, Homo sapiens
tgcatttctctgtcactgtaactat

SEQ ID NO: 512 - DNA, 25 bp, Homo sapiens
aaaagccatagccgaggactgtccc

SEQ ID NO: 513 - DNA, 25 bp, Homo sapiens
aacaccgccagcgtggattttccaa

SEQ ID NO: 514 - DNA, 25 bp, Homo sapiens
accaccagaatgcagttccagctta

SEQ ID NO: 515 - DNA, 25 bp, Homo sapiens
cagaccactctagccacagtatatt

SEQ ID NO: 516 - DNA, 25 bp, Homo sapiens
ccgtggactgcgtctaggtcatgtg

SEQ ID NO: 517 - DNA, 25 bp, Homo sapiens
ctctgtggtcccttcaaagttgtta

SEQ ID NO: 518 - DNA, 25 bp, Homo sapiens
gaaaggcgatctcttcactgtgaaa

SEQ ID NO: 519 - DNA, 25 bp, Homo sapiens
gagagtctctggagcccaggatgcc

SEQ ID NO: 520 - DNA, 25 bp, Homo sapiens
ggatgccagcatgtgccaatgactg

SEQ ID NO: 521 - DNA, 25 bp, Homo sapiens
tgccaatgactgtcaccttcatctc

SEQ ID NO: 522 - DNA, 25 bp, Homo sapiens
tggaaagtaagtctcgctcttgcca

SEQ ID NO: 523 - DNA, 25 bp, Homo sapiens
aaaatcttcgcagatctttgatatc

SEQ ID NO: 524 - DNA, 25 bp, Homo sapiens
aaggcctgtgacagaattcgctgtt

SEQ ID NO: 525 - DNA, 25 bp, Homo sapiens
atgggcattgcaagtgccaccgtgc

SEQ ID NO: 526 - DNA, 25 bp, Homo sapiens
cctgcttcccatgggcattgcaagt

SEQ ID NO: 527 - DNA, 25 bp, Homo sapiens
ctccccaacaggtctctcttgttgg

SEQ ID NO: 528 - DNA, 25 bp, Homo sapiens
ctccgcaataattcaccagaccaga

SEQ ID NO: 529 - DNA, 25 bp, Homo sapiens
ctgccccagggcacataagagcaaa

SEQ ID NO: 530 - DNA, 25 bp, Homo sapiens
ggatgactctgcaaaagtgaccccc

SEQ ID NO: 531 - DNA, 25 bp, Homo sapiens
gtatactgtatcagcagctttgtgt

SEQ ID NO: 532 - DNA, 25 bp, Homo sapiens
taacttggggatggtctcccctgcc

SEQ ID NO: 533 - DNA, 25 bp, Homo sapiens
tactgaggtaacttccacgtagccc

SEQ ID NO: 534 - DNA, 25 bp, Homo sapiens
aattcagactctcttttcattatgt

SEQ ID NO: 535 - DNA, 25 bp, Homo sapiens
agagtcatagtctaggatcctgaga

SEQ ID NO: 536 - DNA, 25 bp, Homo sapiens
gattgagccaaattctgttgtcagt

SEQ ID NO: 537 - DNA, 25 bp, Homo sapiens
gttctaagcatgcagttctcacctc

SEQ ID NO: 538 - DNA, 25 bp, Homo sapiens
tagctaatttgccattttacttaaa

SEQ ID NO: 539 - DNA, 25 bp, Homo sapiens
tagctggggagcctaaatttagttc

SEQ ID NO: 540 - DNA, 25 bp, Homo sapiens
tcctttcttagcttgatattgccta

SEQ ID NO: 541 - DNA, 25 bp, Homo sapiens
tgtcaccattcacttgcattgtaaa

SEQ ID NO: 542 - DNA, 25 bp, Homo sapiens
ttctgttgtcagttctaagcatgca

SEQ ID NO: 543 - DNA, 25 bp, Homo sapiens
ttgatattgcctagctttgttgttt

SEQ ID NO: 544 - DNA, 25 bp, Homo sapiens
ttttctttgtctgttgttggcatag

SEQ ID NO: 545 - DNA, 25 bp, Homo sapiens
aatatctagttctcagagcatttgg

SEQ ID NO: 546 - DNA, 25 bp, Homo sapiens
acttgttgacaatgcactgacttta

SEQ ID NO: 547 - DNA, 25 bp, Homo sapiens
atataaaatctgtcctttcctacct

SEQ ID NO: 548 - DNA, 25 bp, Homo sapiens
ctactaatgttgtttgatctgtgtt

SEQ ID NO: 549 - DNA, 25 bp, Homo sapiens
gatctgtgtttgttatactggttgt

SEQ ID NO: 550 - DNA, 25 bp, Homo sapiens
ggagtggcctaaattatctaatgta

SEQ ID NO: 551 - DNA, 25 bp, Homo sapiens
ggttatcttaaatggctacctaaat

SEQ ID NO: 552 - DNA, 25 bp, Homo sapiens
taaccacattcaccttgtaaatgac

SEQ ID NO: 553 - DNA, 25 bp, Homo sapiens
tggctacctaaattgaaatcctttt

SEQ ID NO: 554 - DNA, 25 bp, Homo sapiens
tttatctgtaactgttatccaaaca

SEQ ID NO: 555 - DNA, 25 bp, Homo sapiens
tttcctacctggacatgtcccatta

SEQ ID NO: 556 - DNA, 25 bp, Homo sapiens
aaatagcacatgctctttgcctctc

SEQ ID NO: 557 - DNA, 25 bp, Homo sapiens
aggtgactttctgaaactcccttgt

SEQ ID NO: 558 - DNA, 25 bp, Homo sapiens
agtagatctgctttctgttcatctc

SEQ ID NO: 559 - DNA, 25 bp, Homo sapiens
ccctggatgcgcaagctgcacataa

SEQ ID NO: 560 - DNA, 25 bp, Homo sapiens
cgtccctgagtatctgagcgtttaa

SEQ ID NO: 561 - DNA, 25 bp, Homo sapiens
cgttacctgacccgcagaaggagga

SEQ ID NO: 562 - DNA, 25 bp, Homo sapiens
gttcatctctttgtcctgaatggct

SEQ ID NO: 563 - DNA, 25 bp, Homo sapiens
gtttattgccattatagcgcctgta

SEQ ID NO: 564 - DNA, 25 bp, Homo sapiens
tagcggatcccgcgtagtgtcagta

SEQ ID NO: 565 - DNA, 25 bp, Homo sapiens
tcatgacaacataggcggcccggaa

SEQ ID NO: 566 - DNA, 25 bp, Homo sapiens
tcgttgccctaattcatcttttaat

SEQ ID NO: 567 - DNA, 25 bp, Homo sapiens
agcataaatccccttttcaggaaga

SEQ ID NO: 568 - DNA, 25 bp, Homo sapiens
agcctttaagtgctgcttctgtcag

SEQ ID NO: 569 - DNA, 25 bp, Homo sapiens
atcccatttgaggtataagtcactc

SEQ ID NO: 570 - DNA, 25 bp, Homo sapiens
cagtgttagcataaatccccttttc

SEQ ID NO: 571 - DNA, 25 bp, Homo sapiens
ccacagcatttgtactgttcctttt

SEQ ID NO: 572 - DNA, 25 bp, Homo sapiens
gagctttaccctagttgaacataca

SEQ ID NO: 573 - DNA, 25 bp, Homo sapiens
gatttacacatactgtttcattcta

SEQ ID NO: 574 - DNA, 25 bp, Homo sapiens
ggaagttaaaatatctctacacgta

SEQ ID NO: 575 - DNA, 25 bp, Homo sapiens
gtgacatgctcttgagctttaccct

SEQ ID NO: 576 - DNA, 25 bp, Homo sapiens
gtgctgcttctgtcagtcaaacgtt

SEQ ID NO: 577 - DNA, 25 bp, Homo sapiens
ttcaaagtgcccagactgtgtacaa

SEQ ID NO: 578 - DNA, 25 bp, Homo sapiens
actgaattctccaacagactctacc

SEQ ID NO: 579 - DNA, 25 bp, Homo sapiens
caggctcaccttaaaatcagccctt

SEQ ID NO: 580 - DNA, 25 bp, Homo sapiens
ccactgtcactcctcagaaagctaa

SEQ ID NO: 581 - DNA, 25 bp, Homo sapiens
gaacagacgatccatgctaatattg

SEQ ID NO: 582 - DNA, 25 bp, Homo sapiens
gaagccttcattgctgatcttgaca

SEQ ID NO: 583 - DNA, 25 bp, Homo sapiens
gaggacctgctaaaatcagctacta

SEQ ID NO: 584 - DNA, 25 bp, Homo sapiens
gcttcagaaagttccgaggacctgc

SEQ ID NO: 585 - DNA, 25 bp, Homo sapiens
ggacaaagacgtgcactcaaccttc

SEQ ID NO: 586 - DNA, 25 bp, Homo sapiens
tagcagtaagctttcccattataat

SEQ ID NO: 587 - DNA, 25 bp, Homo sapiens
tcagctactagaatctgctgccaga

SEQ ID NO: 588 - DNA, 25 bp, Homo sapiens
tctgggtcctttcatcataagggag

SEQ ID NO: 589 - DNA, 25 bp, Homo sapiens
aatgcatctggctactttttcatgt

SEQ ID NO: 590 - DNA, 25 bp, Homo sapiens
acaagactttaccatacacgcaact

SEQ ID NO: 591 - DNA, 25 bp, Homo sapiens
actggcattactcagcaggagcccc

SEQ ID NO: 592 - DNA, 25 bp, Homo sapiens
agaaactaatccttactatcctatt

SEQ ID NO: 593 - DNA, 25 bp, Homo sapiens
attaggataccacttttcattgcaa

SEQ ID NO: 594 - DNA, 25 bp, Homo sapiens
caagttcaagggctctttctccctg

SEQ ID NO: 595 - DNA, 25 bp, Homo sapiens
ctgcatcagttcactgctgcatgtt

SEQ ID NO: 596 - DNA, 25 bp, Homo sapiens
gaaacactttctcacttacagggga

SEQ ID NO: 597 - DNA, 25 bp, Homo sapiens
ggatttcacggagacagcaaccaga

SEQ ID NO: 598 - DNA, 25 bp, Homo sapiens
tggcttctctttacagctttgtttc

SEQ ID NO: 599 - DNA, 25 bp, Homo sapiens
ttcatatgtccccactggcattact

SEQ ID NO: 600 - DNA, 25 bp, Homo sapiens
aagaatcccaattgcaccttctgtt

SEQ ID NO: 601 - DNA, 25 bp, Homo sapiens
actttcgctctctaatcagcatttc

SEQ ID NO: 602 - DNA, 25 bp, Homo sapiens
attgtgtcggaccctacttttgaga

SEQ ID NO: 603 - DNA, 25 bp, Homo sapiens
gcaacctaaattactttcgctctct

SEQ ID NO: 604 - DNA, 25 bp, Homo sapiens
gcaccttctgtttctgacagtcaca

SEQ ID NO: 605 - DNA, 25 bp, Homo sapiens
gcatcaccctgctaatacataataa

SEQ ID NO: 606 - DNA, 25 bp, Homo sapiens
tagtctctggcctgtggatccagtg

SEQ ID NO: 607 - DNA, 25 bp, Homo sapiens
tcttacctgccaacatattcaccat

SEQ ID NO: 608 - DNA, 25 bp, Homo sapiens
tggatccagtgctattctgtcacca

SEQ ID NO: 609 - DNA, 25 bp, Homo sapiens
tgggaactggctattccttgtcccg

SEQ ID NO: 610 - DNA, 25 bp, Homo sapiens
ttgataagcactcctagtctctggc

SEQ ID NO: 611 - DNA, 25 bp, Homo sapiens
atggtgctttatatttagattggaa

SEQ ID NO: 612 - DNA, 25 bp, Homo sapiens
attattgcttatgtgccctgttcaa

SEQ ID NO: 613 - DNA, 25 bp, Homo sapiens
attccagcatcttaccttcatatgc

SEQ ID NO: 614 - DNA, 25 bp, Homo sapiens
gaaagcccgctttagtcaatacttt

SEQ ID NO: 615 - DNA, 25 bp, Homo sapiens
gaaagctgtttgtcgtaacttgaaa

SEQ ID NO: 616 - DNA, 25 bp, Homo sapiens
ggcagttgtctgcattaacctgttc

SEQ ID NO: 617 - DNA, 25 bp, Homo sapiens
ggccttttctattcctgtaatgaaa

SEQ ID NO: 618 - DNA, 25 bp, Homo sapiens
tatcttttactatgggagtcactat

SEQ ID NO: 619 - DNA, 25 bp, Homo sapiens
tatgtagtgtgctttttgtcccttt

SEQ ID NO: 620 - DNA, 25 bp, Homo sapiens
tatttgtttctggtctttgttaagt

SEQ ID NO: 621 - DNA, 25 bp, Homo sapiens
tgttattggccttttctattcctgt

SEQ ID NO: 622 - DNA, 25 bp, Homo sapiens
aaacctcagttctgtcacttcttac

SEQ ID NO: 623 - DNA, 25 bp, Homo sapiens
aagtgattcgggcatatttgtgtga

SEQ ID NO: 624 - DNA, 25 bp, Homo sapiens
agctcaaatttcagtccacatatga

SEQ ID NO: 625 - DNA, 25 bp, Homo sapiens
caatggcttttctaacaacctcagt

SEQ ID NO: 626 - DNA, 25 bp, Homo sapiens
catcatccagaccattaatagaatc

SEQ ID NO: 627 - DNA, 25 bp, Homo sapiens
gaaatgtgagagaggctcgccacta

SEQ ID NO: 628 - DNA, 25 bp, Homo sapiens
gaggctcgccactaagtattctaaa

SEQ ID NO: 629 - DNA, 25 bp, Homo sapiens
gatactcagctgtcatgtttataat

SEQ ID NO: 630 - DNA, 25 bp, Homo sapiens
gctctcagtctgtgtcatgtaagga

SEQ ID NO: 631 - DNA, 25 bp, Homo sapiens
tagttgcttttgatactcagctgtc

SEQ ID NO: 632 - DNA, 25 bp, Homo sapiens
ttcaaaaagctctcagtctgtgtca

SEQ ID NO: 633 - DNA, 25 bp, Homo sapiens
aaactgctgcatactttgacaagga

SEQ ID NO: 634 - DNA, 25 bp, Homo sapiens
aaagatcaccttgtattctctttac

SEQ ID NO: 635 - DNA, 25 bp, Homo sapiens
aatctatatttgtcttccgatcaac

SEQ ID NO: 636 - DNA, 25 bp, Homo sapiens
atacctggtttacttctttagcatt

SEQ ID NO: 637 - DNA, 25 bp, Homo sapiens
atccgacttgaatattcctggactt

SEQ ID NO: 638 - DNA, 25 bp, Homo sapiens
cagacagtctgttatgcactgtggt

SEQ ID NO: 639 - DNA, 25 bp, Homo sapiens
gatggtgcttggtgagtcttggttc

SEQ ID NO: 640 - DNA, 25 bp, Homo sapiens
gccaagggggtgactggaagttgtg

SEQ ID NO: 641 - DNA, 25 bp, Homo sapiens
ggaagaccagaattcccttgaattg

SEQ ID NO: 642 - DNA, 25 bp, Homo sapiens
ggtttattcccaagtatgccttaag

SEQ ID NO: 643 - DNA, 25 bp, Homo sapiens
ttttctatatagttccttgccttaa

SEQ ID NO: 644 - DNA, 25 bp, Homo sapiens
agaaaacacctgtgaagctggaggt

SEQ ID NO: 645 - DNA, 25 bp, Homo sapiens
agttgacttccatcagtgttgagcc

SEQ ID NO: 646 - DNA, 25 bp, Homo sapiens
ataagaaaatctccttgtggtgaag

SEQ ID NO: 647 - DNA, 25 bp, Homo sapiens
cactcatcgctgttccgaacaagtc

SEQ ID NO: 648 - DNA, 25 bp, Homo sapiens
gaatgtctaagtgaagggaccagtt

SEQ ID NO: 649 - DNA, 25 bp, Homo sapiens
gagattgttaagcagttgacttcca

SEQ ID NO: 650 - DNA, 25 bp, Homo sapiens
ggtgtgtgctgactggattcagagg

SEQ ID NO: 651 - DNA, 25 bp, Homo sapiens
ggttcagagacatgggatcgtttcc

SEQ ID NO: 652 - DNA, 25 bp, Homo sapiens
tccatcagtgttgagccaggaattg

SEQ ID NO: 653 - DNA, 25 bp, Homo sapiens
tcgctgttccgaacaagtcagccag

SEQ ID NO: 654 - DNA, 25 bp, Homo sapiens
tgaagctggaggtgaccattcacca

SEQ ID NO: 655 - DNA, 25 bp, Homo sapiens
aaacagatcacatgtgggcccgtgt

SEQ ID NO: 656 - DNA, 25 bp, Homo sapiens
aagagatccaggtctttgcgtttcc

SEQ ID NO: 657 - DNA, 25 bp, Homo sapiens
aagcacggtgtgttctgcttttctt

SEQ ID NO: 658 - DNA, 25 bp, Homo sapiens
agacgagggactctttgtcacgtgg

SEQ ID NO: 659 - DNA, 25 bp, Homo sapiens
cacctaatttattgccgtgcgtcct

SEQ ID NO: 660 - DNA, 25 bp, Homo sapiens
gccggggaagcacggtgtgttctgc

SEQ ID NO: 661 - DNA, 25 bp, Homo sapiens
gtgactgcttttgtacctttgcaat

SEQ ID NO: 662 - DNA, 25 bp, Homo sapiens
tgcggccaccacctaatttattgcc

SEQ ID NO: 663 - DNA, 25 bp, Homo sapiens
tgtgctacttggcagttccatttca

SEQ ID NO: 664 - DNA, 25 bp, Homo sapiens
ttcttggtgtccacgtcttgtgggc

SEQ ID NO: 665 - DNA, 25 bp, Homo sapiens
ttttgtgctgctttttatcatgata

SEQ ID NO: 666 - DNA, 25 bp, Homo sapiens
aaatagcactgttccagtcagccac

SEQ ID NO: 667 - DNA, 25 bp, Homo sapiens
aatgaagtgttcccaaccttatgtt

SEQ ID NO: 668 - DNA, 25 bp, Homo sapiens
actccatattttatgctggttgtct

SEQ ID NO: 669 - DNA, 25 bp, Homo sapiens
actgtattcagttattttgcccttt

SEQ ID NO: 670 - DNA, 25 bp, Homo sapiens
actttatgacgtctgaggcacaccc

SEQ ID NO: 671 - DNA, 25 bp, Homo sapiens
atggtgtttggcttttcttaacatt

SEQ ID NO: 672 - DNA, 25 bp, Homo sapiens
gcctttcagtgcattactatgggag

SEQ ID NO: 673 - DNA, 25 bp, Homo sapiens
gtcagccactactttatgacgtctg

SEQ ID NO: 674 - DNA, 25 bp, Homo sapiens
gttgtctgcaagcttgtgcgatgtt

SEQ ID NO: 675 - DNA, 25 bp, Homo sapiens
tgaggtactttcttcaaatgctttg

SEQ ID NO: 676 - DNA, 25 bp, Homo sapiens
ttttgccctttattgaggaaccaga

SEQ ID NO: 677 - DNA, 25 bp, Homo sapiens
aatgcaccggtttggattcaggcac

SEQ ID NO: 678 - DNA, 25 bp, Homo sapiens
ataactccaacctgtttgattccgt

SEQ ID NO: 679 - DNA, 25 bp, Homo sapiens
cttcccccaataatgcagctgtata

SEQ ID NO: 680 - DNA, 25 bp, Homo sapiens
cttctgcgtctgtgaggccaatgca

SEQ ID NO: 681 - DNA, 25 bp, Homo sapiens
gactaagattcctgcattttgactc

SEQ ID NO: 682 - DNA, 25 bp, Homo sapiens
gaggccaatgcaaatccttttcagg

SEQ ID NO: 683 - DNA, 25 bp, Homo sapiens
gatttgactgtgtgctttttcaagt

SEQ ID NO: 684 - DNA, 25 bp, Homo sapiens
gtttgattccgtctgttttctaaat

SEQ ID NO: 685 - DNA, 25 bp, Homo sapiens
tcccccttcctgatgatgagtgaga

SEQ ID NO: 686 - DNA, 25 bp, Homo sapiens
tgagaactttcggggtcagtgccct

SEQ ID NO: 687 - DNA, 25 bp, Homo sapiens
ttttttgcttaccctcatcaacaga

SEQ ID NO: 688 - DNA, 25 bp, Homo sapiens
caggagtgcacggcgcagatgtata

SEQ ID NO: 689 - DNA, 25 bp, Homo sapiens
gataactttaatcctcacttctcag

SEQ ID NO: 690 - DNA, 25 bp, Homo sapiens
gcacatcagtaaatatctgcagtct

SEQ ID NO: 691 - DNA, 25 bp, Homo sapiens
gtcgtttgataactttaatcctcac

SEQ ID NO: 692 - DNA, 25 bp, Homo sapiens
gtgcacggcgcagatgtatatacat

SEQ ID NO: 693 - DNA, 25 bp, Homo sapiens
gtggttgccctcaggatggtattca

SEQ ID NO: 694 - DNA, 25 bp, Homo sapiens
taatacaaatgggctctttgttttt

SEQ ID NO: 695 - DNA, 25 bp, Homo sapiens
tctgcagtcttgtgcacatggtggt

SEQ ID NO: 696 - DNA, 25 bp, Homo sapiens
ttaatcctcacttctcaggaaacat

SEQ ID NO: 697 - DNA, 25 bp, Homo sapiens
ttctcaggaaacattgcacatcagt

SEQ ID NO: 698 - DNA, 25 bp, Homo sapiens
ttggtctgtcgccaaggcaggagtg

SEQ ID NO: 699 - DNA, 25 bp, Homo sapiens
aaatggtagcaacagacagccctct

SEQ ID NO: 700 - DNA, 25 bp, Homo sapiens
agcatggaattgtctacgccttttg

SEQ ID NO: 701 - DNA, 25 bp, Homo sapiens
cttttgattggaatgcactccccct

SEQ ID NO: 702 - DNA, 25 bp, Homo sapiens
gaaagaccatcaatcctgggtttta

SEQ ID NO: 703 - DNA, 25 bp, Homo sapiens
ggaggtgaacgtctttgtggctatg

SEQ ID NO: 704 - DNA, 25 bp, Homo sapiens
ggtgaaagtcggcctgtgagtaaca

SEQ ID NO: 705 - DNA, 25 bp, Homo sapiens
taggttcagggtcagttaccagcct

SEQ ID NO: 706 - DNA, 25 bp, Homo sapiens
tcaccattctttcccataaggcttg

SEQ ID NO: 707 - DNA, 25 bp, Homo sapiens
tcagttccgtgctctgtaaaaccga

SEQ ID NO: 708 - DNA, 25 bp, Homo sapiens
tgctttacctaccttccaaggttat

SEQ ID NO: 709 - DNA, 25 bp, Homo sapiens
ttgtacataagccctaccttttgtc

SEQ ID NO: 710 - DNA, 25 bp, Homo sapiens
acacctatccaggacctagtttcca

SEQ ID NO: 711 - DNA, 25 bp, Homo sapiens
aggatagggcaatcattcccaagga

SEQ ID NO: 712 - DNA, 25 bp, Homo sapiens
attttgttggaagctccattcccaa

SEQ ID NO: 713 - DNA, 25 bp, Homo sapiens
caggacctagtttccatgaccatgc

SEQ ID NO: 714 - DNA, 25 bp, Homo sapiens
cccttttctcattgtccatgtgatc

SEQ ID NO: 715 - DNA, 25 bp, Homo sapiens
gaacgatggctgctaacacctatcc

SEQ ID NO: 716 - DNA, 25 bp, Homo sapiens
gctccattcccaaagcttaacactt

SEQ ID NO: 717 - DNA, 25 bp, Homo sapiens
taaacaggacagttccatgcaggga

SEQ ID NO: 718 - DNA, 25 bp, Homo sapiens
tcctttgcccacttcttaaatgtta

SEQ ID NO: 719 - DNA, 25 bp, Homo sapiens
ttctccaagttaagtttcagccctt

SEQ ID NO: 720 - DNA, 25 bp, Homo sapiens
tttatgtagtcttatccactgccac

SEQ ID NO: 721 - DNA, 25 bp, Homo sapiens
aaacttcttaattatggaggtacat

SEQ ID NO: 722 - DNA, 25 bp, Homo sapiens
aagaagaaatcttaccttgctctgt

SEQ ID NO: 723 - DNA, 25 bp, Homo sapiens
aagcccatttctaattggtgattgt

SEQ ID NO: 724 - DNA, 25 bp, Homo sapiens
agaaatcttaccttgctctgtatct

SEQ ID NO: 725 - DNA, 25 bp, Homo sapiens
atggaggtacatctccaatacctaa

SEQ ID NO: 726 - DNA, 25 bp, Homo sapiens
catccccctgtcaaaatgtttgctt

SEQ ID NO: 727 - DNA, 25 bp, Homo sapiens
ggcacacactgtagtttcctaagca

SEQ ID NO: 728 - DNA, 25 bp, Homo sapiens
gtacatctccaatacctaaaattaa

SEQ ID NO: 729 - DNA, 25 bp, Homo sapiens
gtattgtcatttaagcccatttcta

SEQ ID NO: 730 - DNA, 25 bp, Homo sapiens
gtttcctaagcagtttgttctaatt

SEQ ID NO: 731 - DNA, 25 bp, Homo sapiens
ttcacatccccctgtcaaaatgttt

SEQ ID NO: 732 - DNA, 25 bp, Homo sapiens
aaagacctcataccatacctgtaat

SEQ ID NO: 733 - DNA, 25 bp, Homo sapiens
aatggtagtaggtgtgcctctctcc

SEQ ID NO: 734 - DNA, 25 bp, Homo sapiens
attgccactactgtgaggtttgggt

SEQ ID NO: 735 - DNA, 25 bp, Homo sapiens
cagttgcaggtagctactctggaaa

SEQ ID NO: 736 - DNA, 25 bp, Homo sapiens
cctctctcccatgaacggatatcgc

SEQ ID NO: 737 - DNA, 25 bp, Homo sapiens
gtcagaacccataacaacaggccag

SEQ ID NO: 738 - DNA, 25 bp, Homo sapiens
gtcttagtccccttaatggtagtag

SEQ ID NO: 739 - DNA, 25 bp, Homo sapiens
gtgtagctgaacttccttagtatca

SEQ ID NO: 740 - DNA, 25 bp, Homo sapiens
tcttcttagccaaatacttctcctt

SEQ ID NO: 741 - DNA, 25 bp, Homo sapiens
tgccgcactcttagtttttttgccc

SEQ ID NO: 742 - DNA, 25 bp, Homo sapiens
ttgataattttcgtcttagtcccct

SEQ ID NO: 743 - DNA, 25 bp, Homo sapiens
aactctgtatactgtatcagcagct

SEQ ID NO: 744 - DNA, 25 bp, Homo sapiens
aattcaccagaccagaagccactgg

SEQ ID NO: 745 - DNA, 25 bp, Homo sapiens
acacccaggaaaagtctgcagaccc

SEQ ID NO: 746 - DNA, 25 bp, Homo sapiens
acatgtccctggagttgcttccagc

SEQ ID NO: 747 - DNA, 25 bp, Homo sapiens
actgtatcagcagctttgtgtaaaa

SEQ ID NO: 748 - DNA, 25 bp, Homo sapiens
atgggcattgcaagtgccaccgtgc

SEQ ID NO: 749 - DNA, 25 bp, Homo sapiens
caccagaccagaagccactggtgta

SEQ ID NO: 750 - DNA, 25 bp, Homo sapiens
cagaagccactggtgtacagagaac

SEQ ID NO: 751 - DNA, 25 bp, Homo sapiens
ccactggtgtacagagaacacttaa

SEQ ID NO: 752 - DNA, 25 bp, Homo sapiens
cccaaagggggcacatgtccctgga

SEQ ID NO: 753 - DNA, 25 bp, Homo sapiens
cgcaataattcaccagaccagaagc

SEQ ID NO: 754 - DNA, 25 bp, Homo sapiens
cttcccatgggcattgcaagtgcca

SEQ ID NO: 755 - DNA, 25 bp, Homo sapiens
gaggtaacttccacgtagccccttg

SEQ ID NO: 756 - DNA, 25 bp, Homo sapiens
gcctggctctgcacacccaggaaaa

SEQ ID NO: 757 - DNA, 25 bp, Homo sapiens
gcctgtgacagaattcgctgttaag

SEQ ID NO: 758 - DNA, 25 bp, Homo sapiens
tttgatatcgtactgaggtaacttc

SEQ ID NO: 759 - DNA, 25 bp, Homo sapiens
ataagtagccgcctggttactgtgt

SEQ ID NO: 760 - DNA, 25 bp, Homo sapiens
cgcctggttactgtgtcctgtaaaa

SEQ ID NO: 761 - DNA, 25 bp, Homo sapiens
aaaatacagacacttgacccttggt

SEQ ID NO: 762 - DNA, 25 bp, Homo sapiens
ccttggtgtagcttctgttcaactt

SEQ ID NO: 763 - DNA, 25 bp, Homo sapiens
tggatgggtctgatttcttggccct

SEQ ID NO: 764 - DNA, 25 bp, Homo sapiens
ttcttggccctcttcttgaattggc

SEQ ID NO: 765 - DNA, 25 bp, Homo sapiens
gaattggccatatacagggtccctg

SEQ ID NO: 766 - DNA, 25 bp, Homo sapiens
ccagtggactgaaggctttgtctaa

SEQ ID NO: 767 - DNA, 25 bp, Homo sapiens
gatgtgggggagggcggttttatct

SEQ ID NO: 768 - DNA, 25 bp, Homo sapiens
ttgaggttttgatctctgggtaaag

SEQ ID NO: 769 - DNA, 25 bp, Homo sapiens
gaggccgtttatctttgtaaacacg

SEQ ID NO: 770 - DNA, 25 bp, Homo sapiens
ggtaggtggtgattttgaggctgta

SEQ ID NO: 771 - DNA, 25 bp, Homo sapiens
tgaggctgtaacatgcccagaagct

SEQ ID NO: 772 - DNA, 25 bp, Homo sapiens
gaagctgttgtggccgacacttcaa

SEQ ID NO: 773 - DNA, 25 bp, Homo sapiens
gtggccgacacttcaacaataggga

SEQ ID NO: 774 - DNA, 25 bp, Homo sapiens
atatccctactgacagtaactacct

SEQ ID NO: 775 - DNA, 25 bp, Homo sapiens
gtaactacctgtcacatatttctct

SEQ ID NO: 776 - DNA, 25 bp, Homo sapiens
cttttgggtggtggggcttgatgta

SEQ ID NO: 777 - DNA, 25 bp, Homo sapiens
ggcatggtttgcggaggttagattt

SEQ ID NO: 778 - DNA, 25 bp, Homo sapiens
gtgaattgtgctctgatggttaaaa

SEQ ID NO: 779 - DNA, 25 bp, Homo sapiens
agattgtcaagcattccgtattaac

SEQ ID NO: 780 - DNA, 25 bp, Homo sapiens
attgattcccatctggcatattcta

SEQ ID NO: 781 - DNA, 25 bp, Homo sapiens
actgtgatataggtactctgattta

SEQ ID NO: 782 - DNA, 25 bp, Homo sapiens
aactttggacatcctgtgatctgtt

SEQ ID NO: 783 - DNA, 25 bp, Homo sapiens
gggggtgggaaatttagctgactag

SEQ ID NO: 784 - DNA, 25 bp, Homo sapiens
gacaaacatgtaaacctattttcct

SEQ ID NO: 785 - DNA, 25 bp, Homo sapiens
aaatgtcccacttgaataacgtaat

SEQ ID NO: 786 - DNA, 25 bp, Homo sapiens
ctgtcttctgggagttatcaatttt

SEQ ID NO: 787 - DNA, 25 bp, Homo sapiens
gaaagtgcactactgcctcatgtaa

SEQ ID NO: 788 - DNA, 25 bp, Homo sapiens
tactgcctcatgtaaagactcttgc

SEQ ID NO: 789 - DNA, 25 bp, Homo sapiens
aagactcttgcacgcagagccttta

SEQ ID NO: 790 - DNA, 25 bp, Homo sapiens
gcacgcagagcctttaagtgactaa

SEQ ID NO: 791 - DNA, 25 bp, Homo sapiens
tgaatacttcaattgtgcctctcaa

SEQ ID NO: 792 - DNA, 25 bp, Homo sapiens
ttttgctagtgttgaattttcttct

SEQ ID NO: 793 - DNA, 25 bp, Homo sapiens
caagcccaagactgcttaacttcca

SEQ ID NO: 794 - DNA, 25 bp, Homo sapiens
ggtatgggagtgggctctatggggt

SEQ ID NO: 795 - DNA, 25 bp, Homo sapiens
ctctatggggtggtctgcacccatc

SEQ ID NO: 796 - DNA, 25 bp, Homo sapiens
tgggactcttttccctaaatcctgc

SEQ ID NO: 797 - DNA, 25 bp, Homo sapiens
ggcagggtgcacagcattagtttca

SEQ ID NO: 798 - DNA, 25 bp, Homo sapiens
cgcccccaccttgaatagctaaagt

SEQ ID NO: 799 - DNA, 25 bp, Homo sapiens
gagttgttgacgtctaactccttcc

SEQ ID NO: 800 - DNA, 25 bp, Homo sapiens
gtctaactccttccattaaattaat

SEQ ID NO: 801 - DNA, 25 bp, Homo sapiens
aagtactgacctcctaatatttaag

SEQ ID NO: 802 - DNA, 25 bp, Homo sapiens
gattcttttatattccattgttcag

SEQ ID NO: 803 - DNA, 25 bp, Homo sapiens
tctgctgaatactatacccttcagc

SEQ ID NO: 804 - DNA, 25 bp, Homo sapiens
gaatactatacccttcagcaatggc

SEQ ID NO: 805 - DNA, 25 bp, Homo sapiens
tcagcaatggctactagaaggacga

SEQ ID NO: 806 - DNA, 25 bp, Homo sapiens
ctagaaggacgaacaattgccctcc

SEQ ID NO: 807 - DNA, 25 bp, Homo sapiens
aaggacgaacaattgccctcctttg

SEQ ID NO: 808 - DNA, 25 bp, Homo sapiens
ttggaagtacggctaatagaagccc

SEQ ID NO: 809 - DNA, 25 bp, Homo sapiens
atagaagccctagatccgaataaga

SEQ ID NO: 810 - DNA, 25 bp, Homo sapiens
gccctagatccgaataagatccgaa

SEQ ID NO: 811 - DNA, 25 bp, Homo sapiens
taagaatatgtaatggaccaggcgc

SEQ ID NO: 812 - DNA, 25 bp, Homo sapiens
atgtaatggaccaggcgcagtgcct

SEQ ID NO: 813 - DNA, 25 bp, Homo sapiens
tgatgacagaagtgtgagaccagcc

SEQ ID NO: 814 - DNA, 25 bp, Homo sapiens
caacattgaggcagggctcactctc

SEQ ID NO: 815 - DNA, 25 bp, Homo sapiens
agggctcactctcctaaattgtagg

SEQ ID NO: 816 - DNA, 25 bp, Homo sapiens
gacagatctaactttcctagtggaa

SEQ ID NO: 817 - DNA, 25 bp, Homo sapiens
gtttcagcatgtgtgtacacctatg

SEQ ID NO: 818 - DNA, 25 bp, Homo sapiens
tacacctatgaaaccaccacagtca

SEQ ID NO: 819 - DNA, 25 bp, Homo sapiens
accacagtcaagatatccaacacaa

SEQ ID NO: 820 - DNA, 25 bp, Homo sapiens
aagattgtccctttataatcctcaa

SEQ ID NO: 821 - DNA, 25 bp, Homo sapiens
tataatcctcaatttttccttatct

SEQ ID NO: 822 - DNA, 25 bp, Homo sapiens
tttccacaattcacaagcaacagca

SEQ ID NO: 823 - DNA, 25 bp, Homo sapiens
ggttaatccattatcttgttgcatg

SEQ ID NO: 824 - DNA, 25 bp, Homo sapiens
gaatcaattgtttgctcatttgtat

SEQ ID NO: 825 - DNA, 25 bp, Homo sapiens
aacctctgtatgcacatgatgggat

SEQ ID NO: 826 - DNA, 25 bp, Homo sapiens
aggacatttgaaaccctaattgtga

SEQ ID NO: 827 - DNA, 25 bp, Homo sapiens
aggcactatgcttttattatataac

SEQ ID NO: 828 - DNA, 25 bp, Homo sapiens
atgcacaatgtcttaagtcttccta

SEQ ID NO: 829 - DNA, 25 bp, Homo sapiens
gatattctcagccctgttaacacta

SEQ ID NO: 830 - DNA, 25 bp, Homo sapiens
gccttgaggatagtcttcatgttca

SEQ ID NO: 831 - DNA, 25 bp, Homo sapiens
gtagtgactcattgtattacttaaa

SEQ ID NO: 832 - DNA, 25 bp, Homo sapiens
gtcttcatgttcaaaggcactatgc

SEQ ID NO: 833 - DNA, 25 bp, Homo sapiens
taaaacttatataacacgctgtatt

SEQ ID NO: 834 - DNA, 25 bp, Homo sapiens
tacatcaccttaacctctgtatgca

SEQ ID NO: 835 - DNA, 25 bp, Homo sapiens
tgaaccacatgatattctcagccct

SEQ ID NO: 836 - DNA, 25 bp, Homo sapiens
actctagagtaatgatggtccctgt

SEQ ID NO: 837 - DNA, 25 bp, Homo sapiens
ataaacaccaacgatggcctctttt

SEQ ID NO: 838 - DNA, 25 bp, Homo sapiens
catatgtatttgaccctgtgggagg

SEQ ID NO: 839 - DNA, 25 bp, Homo sapiens
ccccttcctttgatcatttcatgtg

SEQ ID NO: 840 - DNA, 25 bp, Homo sapiens
gattctcaattgttatgtccactta

SEQ ID NO: 841 - DNA, 25 bp, Homo sapiens
ggagttatgcatagacccactctag

SEQ ID NO: 842 - DNA, 25 bp, Homo sapiens
ggtccctgtggtatatactttctcc

SEQ ID NO: 843 - DNA, 25 bp, Homo sapiens
ggttctcagaagccaaaatacacaa

SEQ ID NO: 844 - DNA, 25 bp, Homo sapiens
gtccacttattcactaggtaaattt

SEQ ID NO: 845 - DNA, 25 bp, Homo sapiens
taaattccttgttgatgtaccctta

SEQ ID NO: 846 - DNA, 25 bp, Homo sapiens
tactttctcctactctagcaaacat

SEQ ID NO: 847 - DNA, 25 bp, Homo sapiens
aggtgagcagtaggtcatccagtcc

SEQ ID NO: 848 - DNA, 25 bp, Homo sapiens
caactcgaagtcatccatggacccc

SEQ ID NO: 849 - DNA, 25 bp, Homo sapiens
cacagcctattccaagcctaaacgg

SEQ ID NO: 850 - DNA, 25 bp, Homo sapiens
cagccaagacgtagatccatccaag

SEQ ID NO: 851 - DNA, 25 bp, Homo sapiens
ccatcccaatggcttatcttacact

SEQ ID NO: 852 - DNA, 25 bp, Homo sapiens
cctttctacttactaccagcaatgc

SEQ ID NO: 853 - DNA, 25 bp, Homo sapiens
gcaaaatacatctcgcctggtacag

SEQ ID NO: 854 - DNA, 25 bp, Homo sapiens
ggacccctgatgattccacagatac

SEQ ID NO: 855 - DNA, 25 bp, Homo sapiens
gggagcagtgtggagagcttgcccc

SEQ ID NO: 856 - DNA, 25 bp, Homo sapiens
tctggatgtccctgagatcgtcata

SEQ ID NO: 857 - DNA, 25 bp, Homo sapiens
tgattactacctcaggaccaacctc

SEQ ID NO: 858 - DNA, 25 bp, Homo sapiens
aggtgagcagtaggtcatccagtcc

SEQ ID NO: 859 - DNA, 25 bp, Homo sapiens
caaaagcctttctacttactaccag

SEQ ID NO: 860 - DNA, 25 bp, Homo sapiens
caactcgaagtcatccatggacccc

SEQ ID NO: 861 - DNA, 25 bp, Homo sapiens
cagccaagacgtagatccatccaag

SEQ ID NO: 862 - DNA, 25 bp, Homo sapiens
ccatcccaatggcttatcttacact

SEQ ID NO: 863 - DNA, 25 bp, Homo sapiens
gacaaggcacttcatgattctctgg

SEQ ID NO: 864 - DNA, 25 bp, Homo sapiens
gaccaacctcagaaaagccaactcg

SEQ ID NO: 865 - DNA, 25 bp, Homo sapiens
gattctctgggaccgttacattttg

SEQ ID NO: 866 - DNA, 25 bp, Homo sapiens
gcaaaatacatctcgcctggtacag

SEQ ID NO: 867 - DNA, 25 bp, Homo sapiens
ggacccctgatgattccacagatac

SEQ ID NO: 868 - DNA, 25 bp, Homo sapiens
tgattactacctcaggaccaacctc

SEQ ID NO: 869 - DNA, 25 bp, Homo sapiens
aagtagtccatcctatacagatagc

SEQ ID NO: 870 - DNA, 25 bp, Homo sapiens
agagggtacatactcctttctgggg

SEQ ID NO: 871 - DNA, 25 bp, Homo sapiens
ccagggaccactgcctggcattatc

SEQ ID NO: 872 - DNA, 25 bp, Homo sapiens
gaatgctccctaccatatagttgac

SEQ ID NO: 873 - DNA, 25 bp, Homo sapiens
gattatgtgtattgatcaccctgca

SEQ ID NO: 874 - DNA, 25 bp, Homo sapiens
gtataaggtgggcttggtccaacag

SEQ ID NO: 875 - DNA, 25 bp, Homo sapiens
tagctgattaactgtattccccttt

SEQ ID NO: 876 - DNA, 25 bp, Homo sapiens
tgatcaccctgcaatcctattatgt

SEQ ID NO: 877 - DNA, 25 bp, Homo sapiens
tgcctggcattatcgcatgctggga

SEQ ID NO: 878 - DNA, 25 bp, Homo sapiens
tggcccctctaccaatagggcagta

SEQ ID NO: 879 - DNA, 25 bp, Homo sapiens
tttcttccatacattagttcccacc

SEQ ID NO: 880 - DNA, 25 bp, Homo sapiens
aaagcagcctgcacagggcaaggcc

SEQ ID NO: 881 - DNA, 25 bp, Homo sapiens
caaaccggcctagacacgaagacca

SEQ ID NO: 882 - DNA, 25 bp, Homo sapiens
cctcgttctctcagttagcagctgg

SEQ ID NO: 883 - DNA, 25 bp, Homo sapiens
gaaacacaatacactgcctcgttct

SEQ ID NO: 884 - DNA, 25 bp, Homo sapiens
gattgtattcctcagtagcacttta

SEQ ID NO: 885 - DNA, 25 bp, Homo sapiens
gcagcgcaccattcatcatttaggc

SEQ ID NO: 886 - DNA, 25 bp, Homo sapiens
ggcaggacacgtatctctgtctgac

SEQ ID NO: 887 - DNA, 25 bp, Homo sapiens
ggcttgtggtttgttgtttactcta

SEQ ID NO: 888 - DNA, 25 bp, Homo sapiens
gtccatgaccgtttgcattcgaaac

SEQ ID NO: 889 - DNA, 25 bp, Homo sapiens
taatctcacggctcttgatctggaa

SEQ ID NO: 890 - DNA, 25 bp, Homo sapiens
ttggcctgacgctggagtgcggtga

SEQ ID NO: 891 - DNA, 25 bp, Homo sapiens
aagtcagcgattatgccggcggtta

SEQ ID NO: 892 - DNA, 25 bp, Homo sapiens
atgtcggtgcacagctgaaagtcag

SEQ ID NO: 893 - DNA, 25 bp, Homo sapiens
attcccgtcagagtttgctttgatt

SEQ ID NO: 894 - DNA, 25 bp, Homo sapiens
cactccatgtggtttcagggttcag

SEQ ID NO: 895 - DNA, 25 bp, Homo sapiens
gccggcggttagaaatgtgccaggg

SEQ ID NO: 896 - DNA, 25 bp, Homo sapiens
gggtgtcattgatgtgggctgagct

SEQ ID NO: 897 - DNA, 25 bp, Homo sapiens
ggttaaaggagtccgcagctcccac

SEQ ID NO: 898 - DNA, 25 bp, Homo sapiens
tgagctggggaacatgtcggtgcac

SEQ ID NO: 899 - DNA, 25 bp, Homo sapiens
tggcctggagggtgacaccatgtca

SEQ ID NO: 900 - DNA, 25 bp, Homo sapiens
ttatttttagctctgcactccatgt

SEQ ID NO: 901 - DNA, 25 bp, Homo sapiens
ttctttattcccctctggactaaag

SEQ ID NO: 902 - DNA, 25 bp, Homo sapiens
cagaggaggctaagcccgggcagct

SEQ ID NO: 903 - DNA, 25 bp, Homo sapiens
cccagtgcccagaaacaatgcctag

SEQ ID NO: 904 - DNA, 25 bp, Homo sapiens
cccagttacacacttccatggtact

SEQ ID NO: 905 - DNA, 25 bp, Homo sapiens
ctcattctcaactccttagactcag

SEQ ID NO: 906 - DNA, 25 bp, Homo sapiens
ctttccatacctgtactcacaacta

SEQ ID NO: 907 - DNA, 25 bp, Homo sapiens
gtactatatatcattccttcagagc

SEQ ID NO: 908 - DNA, 25 bp, Homo sapiens
gtcctttgcaaactcattctcaact

SEQ ID NO: 909 - DNA, 25 bp, Homo sapiens
tatttcctatgtatttgtccagtca

SEQ ID NO: 910 - DNA, 25 bp, Homo sapiens
tccttagactcagtcaagtccccca

SEQ ID NO: 911 - DNA, 25 bp, Homo sapiens
tgaccatttctatctgtgttcacca

SEQ ID NO: 912 - DNA, 25 bp, Homo sapiens
tgttcaccaatgtgttcccagtgcc

SEQ ID NO: 913 - DNA, 25 bp, Homo sapiens
aaattaccttcctattgcatttcct

SEQ ID NO: 914 - DNA, 25 bp, Homo sapiens
agggtagggctgtggtttactcctg

SEQ ID NO: 915 - DNA, 25 bp, Homo sapiens
ctttcctgagcctcttgcttgaatg

SEQ ID NO: 916 - DNA, 25 bp, Homo sapiens
gacatttgtgattctcattttctca

SEQ ID NO: 917 - DNA, 25 bp, Homo sapiens
gatgtactctttgttctctaaaacc

SEQ ID NO: 918 - DNA, 25 bp, Homo sapiens
gcttgaatgtgatttctttctccct

SEQ ID NO: 919 - DNA, 25 bp, Homo sapiens
tattgccacctgtcaaaatcttcat

SEQ ID NO: 920 - DNA, 25 bp, Homo sapiens
tggacaaattctcgaacccattcac

SEQ ID NO: 921 - DNA, 25 bp, Homo sapiens
tgtctaaacccctgaagcctaacac

SEQ ID NO: 922 - DNA, 25 bp, Homo sapiens
ttgctacgtgtattggacctctggc

SEQ ID NO: 923 - DNA, 25 bp, Homo sapiens
tttctccctgagacccataaggttc

SEQ ID NO: 924 - DNA, 25 bp, Homo sapiens
acacgtggctccagatcaaagcggc

SEQ ID NO: 925 - DNA, 25 bp, Homo sapiens
caaagcggccaaggacggagcatcc

SEQ ID NO: 926 - DNA, 25 bp, Homo sapiens
caaagctctgggtgacacgtggctc

SEQ ID NO: 927 - DNA, 25 bp, Homo sapiens
caagaattacaaggagcccgagccg

SEQ ID NO: 928 - DNA, 25 bp, Homo sapiens
ccacctgtgaccccgtggtggagga

SEQ ID NO: 929 - DNA, 25 bp, Homo sapiens
cctcctccaacaacacgtggatctg

SEQ ID NO: 930 - DNA, 25 bp, Homo sapiens
cgtggatctgcatggtttgcctgag

SEQ ID NO: 931 - DNA, 25 bp, Homo sapiens
gacgaccactttgccaaagctctgg

SEQ ID NO: 932 - DNA, 25 bp, Homo sapiens
ggtttgcctgagctttgaacagtca

SEQ ID NO: 933 - DNA, 25 bp, Homo sapiens
gtggtggaggagcatttccgcagga

SEQ ID NO: 934 - DNA, 25 bp, Homo sapiens
tgtggtctcctgaagggagcgcctc

SEQ ID NO: 935 - DNA, 25 bp, Homo sapiens
caagctgtatgtgggcagtcgggtg

SEQ ID NO: 936 - DNA, 25 bp, Homo sapiens
cagtcgggtggtcgccaaatacaaa

SEQ ID NO: 937 - DNA, 25 bp, Homo sapiens
ccatttgccggccactgaaaaagac

SEQ ID NO: 938 - DNA, 25 bp, Homo sapiens
gaaggcacgtggtggaagtcccgag

SEQ ID NO: 939 - DNA, 25 bp, Homo sapiens
gccccatggtactgctcaagagtgg

SEQ ID NO: 940 - DNA, 25 bp, Homo sapiens
gctcaagagtggccagcttatcaag

SEQ ID NO: 941 - DNA, 25 bp, Homo sapiens
ggaagtcccgagttgaggaggtgga

SEQ ID NO: 942 - DNA, 25 bp, Homo sapiens
ggacatagaagacatctcctgccgt

SEQ ID NO: 943 - DNA, 25 bp, Homo sapiens
ggatggcagcctagtcaggatcctc

SEQ ID NO: 944 - DNA, 25 bp, Homo sapiens
tagaggagtatgtcactgcctaccc

SEQ ID NO: 945 - DNA, 25 bp, Homo sapiens
tctcctgccgtgacttcatagagga

SEQ ID NO: 946 - DNA, 25 bp, Homo sapiens
actgacatgcattattttcactgtg

SEQ ID NO: 947 - DNA, 25 bp, Homo sapiens
gaataggccgtgagggtgtgaggaa

SEQ ID NO: 948 - DNA, 25 bp, Homo sapiens
gaatgagggacttccatcagactct

SEQ ID NO: 949 - DNA, 25 bp, Homo sapiens
gagttgccaaactacctgttgtact

SEQ ID NO: 950 - DNA, 25 bp, Homo sapiens
gcaatgatgttcttcctggaattca

SEQ ID NO: 951 - DNA, 25 bp, Homo sapiens
gttcttatcccacccataatgagag

SEQ ID NO: 952 - DNA, 25 bp, Homo sapiens
tacagactgcgaacaacggctttca

SEQ ID NO: 953 - DNA, 25 bp, Homo sapiens
tgcccttcccactttttggaatagg

SEQ ID NO: 954 - DNA, 25 bp, Homo sapiens
ttcagggaaccaagcaactctattt

SEQ ID NO: 955 - DNA, 25 bp, Homo sapiens
tttaggatgttcttatcccacccat

SEQ ID NO: 956 - DNA, 25 bp, Homo sapiens
ttttgctaatggctttgtatgtaac

SEQ ID NO: 957 - DNA, 25 bp, Homo sapiens
acaccactgagtgccatgcagagaa

SEQ ID NO: 958 - DNA, 25 bp, Homo sapiens
acattaagtattttcagcccactag

SEQ ID NO: 959 - DNA, 25 bp, Homo sapiens
agagtccgagtgtctttacaccact

SEQ ID NO: 960 - DNA, 25 bp, Homo sapiens
agcattcaacttttgagggctacca

SEQ ID NO: 961 - DNA, 25 bp, Homo sapiens
aggactgaagtatctactctgggtt

SEQ ID NO: 962 - DNA, 25 bp, Homo sapiens
ctgctgcaccagcttaacatgtggg

SEQ ID NO: 963 - DNA, 25 bp, Homo sapiens
ctttctggatgagctgttctgtctg

SEQ ID NO: 964 - DNA, 25 bp, Homo sapiens
gaaacctacaaggcaccaggctaga

SEQ ID NO: 965 - DNA, 25 bp, Homo sapiens
gaattccaaactttgagccgacgaa

SEQ ID NO: 966 - DNA, 25 bp, Homo sapiens
gatttcagtggccacctgaggaatc

SEQ ID NO: 967 - DNA, 25 bp, Homo sapiens
gtcattttccttgtatctggggagg

SEQ ID NO: 968 - DNA, 25 bp, Homo sapiens
acagaggcatgctaccatacctggt

SEQ ID NO: 969 - DNA, 25 bp, Homo sapiens
acctcctaataccaacaccttgaag

SEQ ID NO: 970 - DNA, 25 bp, Homo sapiens
agagcggtaggttactctgggcaca

SEQ ID NO: 971 - DNA, 25 bp, Homo sapiens
cagaggctctggctcgaaggaagcg

SEQ ID NO: 972 - DNA, 25 bp, Homo sapiens
ccaaggccttccttggtgttgcctc

SEQ ID NO: 973 - DNA, 25 bp, Homo sapiens
gaaggaagcggagggcgtggctgct

SEQ ID NO: 974 - DNA, 25 bp, Homo sapiens
gccttttcttagtgccttagagggc

SEQ ID NO: 975 - DNA, 25 bp, Homo sapiens
ggctgctgagacagccaacacctct

SEQ ID NO: 976 - DNA, 25 bp, Homo sapiens
gtgtgctcttcccagtagagcggta

SEQ ID NO: 977 - DNA, 25 bp, Homo sapiens
ttgcctccattccctggaaaggtct

SEQ ID NO: 978 - DNA, 25 bp, Homo sapiens
ttttacatttcagtgtgctcttccc

SEQ ID NO: 979 - DNA, 25 bp, Homo sapiens
aaccaggccgagaggccacacactt

SEQ ID NO: 980 - DNA, 25 bp, Homo sapiens
atgcatgcgtgtccaggctgaagat

SEQ ID NO: 981 - DNA, 25 bp, Homo sapiens
ccatccccacaaaccaggtaatgcc

SEQ ID NO: 982 - DNA, 25 bp, Homo sapiens
ctgaatgcttcttgctaaccaggcc

SEQ ID NO: 983 - DNA, 25 bp, Homo sapiens
cttctggaagtctctgctcagcaca

SEQ ID NO: 984 - DNA, 25 bp, Homo sapiens
gaagatgcccctatattctgtcaaa

SEQ ID NO: 985 - DNA, 25 bp, Homo sapiens
gaccgtgagggggctcttgatggga

SEQ ID NO: 986 - DNA, 25 bp, Homo sapiens
gctcaaggtgtccaggcttttgggg

SEQ ID NO: 987 - DNA, 25 bp, Homo sapiens
gtcctggtcataactgtgtgctcaa

SEQ ID NO: 988 - DNA, 25 bp, Homo sapiens
gtttgccagcagctatttgcctata

SEQ ID NO: 989 - DNA, 25 bp, Homo sapiens
tgggcctatctgggtgcattatgga

SEQ ID NO: 990 - DNA, 25 bp, Homo sapiens
aacagaatactaagggcccctactg

SEQ ID NO: 991 - DNA, 25 bp, Homo sapiens
aggtgtttgctgaatccaggtctga

SEQ ID NO: 992 - DNA, 25 bp, Homo sapiens
cactgtaccacattatctcttttca

SEQ ID NO: 993 - DNA, 25 bp, Homo sapiens
ccaggtctgagatcacaatcccacc

SEQ ID NO: 994 - DNA, 25 bp, Homo sapiens
ctctgccctcatagaatcctaattg

SEQ ID NO: 995 - DNA, 25 bp, Homo sapiens
gaaacattgaacagccccatttaga

SEQ ID NO: 996 - DNA, 25 bp, Homo sapiens
gaggcccaatctcaactgtagactg

SEQ ID NO: 997 - DNA, 25 bp, Homo sapiens
gattcctagtctagtatccttccca

SEQ ID NO: 998 - DNA, 25 bp, Homo sapiens
ggatccgcatatgagagtcgcacat

SEQ ID NO: 999 - DNA, 25 bp, Homo sapiens
taccgacctcttaggcttggtgtga

SEQ ID NO: 1000 - DNA, 25 bp, Homo sapiens
tcttgtccttgtgctctgtgaaaca

SEQ ID NO: 1001 - DNA, 25 bp, Homo sapiens
acttcgcctgtactgaaagggccaa

SEQ ID NO: 1002 - DNA, 25 bp, Homo sapiens
caaccttctaattaggtaggcctct

SEQ ID NO: 1003 - DNA, 25 bp, Homo sapiens
cccttggatctgttactgcatcact

SEQ ID NO: 1004 - DNA, 25 bp, Homo sapiens
gatcactgctggtcttgatagccat

SEQ ID NO: 1005 - DNA, 25 bp, Homo sapiens
gatgcaatagaacacttcgcctgta

SEQ ID NO: 1006 - DNA, 25 bp, Homo sapiens
gctgaatttgtcaaataccccttcc

SEQ ID NO: 1007 - DNA, 25 bp, Homo sapiens
taatttgagccacattcccaactct

SEQ ID NO: 1008 - DNA, 25 bp, Homo sapiens
taggcctctaggtattctgcagatc

SEQ ID NO: 1009 - DNA, 25 bp, Homo sapiens
tatctcactctgtcattgttaatct

SEQ ID NO: 1010 - DNA, 25 bp, Homo sapiens
tgttactgcatcactagcacttgtg

SEQ ID NO: 1011 - DNA, 25 bp, Homo sapiens
ttccccaccacaccttataaaattg

SEQ ID NO: 1012 - DNA, 25 bp, Homo sapiens
gcttcaggttcttcacctctaagat

SEQ ID NO: 1013 - DNA, 25 bp, Homo sapiens
ggggatgatgaaaacagtacctgtc

SEQ ID NO: 1014 - DNA, 25 bp, Homo sapiens
gtacctgtcatgcagaattgttggg

SEQ ID NO: 1015 - DNA, 25 bp, Homo sapiens
tgctcttttcacttgatatccagta

SEQ ID NO: 1016 - DNA, 25 bp, Homo sapiens
gaaggtgcatgtcttctgtattctg

SEQ ID NO: 1017 - DNA, 25 bp, Homo sapiens
cccatttcttttgcgtgcagtcttt

SEQ ID NO: 1018 - DNA, 25 bp, Homo sapiens
ttgcgtgcagtctttgattcgtaca

SEQ ID NO: 1019 - DNA, 25 bp, Homo sapiens
gaaattgctaccaaactcatttaat

SEQ ID NO: 1020 - DNA, 25 bp, Homo sapiens
ataccaactgttctatatttcttta

SEQ ID NO: 1021 - DNA, 25 bp, Homo sapiens
atcttcagtgattccttttactata

SEQ ID NO: 1022 - DNA, 25 bp, Homo sapiens
aggtttcctttcccatcatatggaa

SEQ ID NO: 1023 - DNA, 25 bp, Homo sapiens
cccattcagacaactgttccccaat

SEQ ID NO: 1024 - DNA, 25 bp, Homo sapiens
ctaccagccatctgcaggggtcagt

SEQ ID NO: 1025 - DNA, 25 bp, Homo sapiens
gtgccacttatgaagagtgccccat

SEQ ID NO: 1026 - DNA, 25 bp, Homo sapiens
aaaaggagactcagctgtcccttgg

SEQ ID NO: 1027 - DNA, 25 bp, Homo sapiens
cttgtgccagtatcccagggcagaa

SEQ ID NO: 1028 - DNA, 25 bp, Homo sapiens
ccttgcgcagagccactgtgagagg

SEQ ID NO: 1029 - DNA, 25 bp, Homo sapiens
tgagaggcggtgggagccaacaccc

SEQ ID NO: 1030 - DNA, 25 bp, Homo sapiens
attaagttcatatccaccttttggg

SEQ ID NO: 1031 - DNA, 25 bp, Homo sapiens
ccaagtgtgtgacttctccatatcc

SEQ ID NO: 1032 - DNA, 25 bp, Homo sapiens
tgggaattttcaatcccctgtgctt

SEQ ID NO: 1033 - DNA, 25 bp, Homo sapiens
tgcttgtctaacgtctgctttaaaa

SEQ ID NO: 1034 - DNA, 25 bp, Homo sapiens
atttaagttgtgattacctgctgca

SEQ ID NO: 1035 - DNA, 25 bp, Homo sapiens
aagtggcatgggggaccctgtgcat

SEQ ID NO: 1036 - DNA, 25 bp, Homo sapiens
gaccctgtgcatctgtgcatttggc

SEQ ID NO: 1037 - DNA, 25 bp, Homo sapiens
tccatttctggacatgacgtctgtg

SEQ ID NO: 1038 - DNA, 25 bp, Homo sapiens
gacgtctgtggtttaagctttgtga

SEQ ID NO: 1039 - DNA, 25 bp, Homo sapiens
aatgtgctttgattcgaagggtctt

SEQ ID NO: 1040 - DNA, 25 bp, Homo sapiens
taatcgtcaaccacttttaaacata

SEQ ID NO: 1041 - DNA, 25 bp, Homo sapiens
agaattcacacaactactttcatga

SEQ ID NO: 1042 - DNA, 25 bp, Homo sapiens
attccaagagtatcccagtattagc

SEQ ID NO: 1043 - DNA, 25 bp, Homo sapiens
atataggcacattaccattcatagt

SEQ ID NO: 1044 - DNA, 25 bp, Homo sapiens
aatttgatgcgatctgctcagtaat

SEQ ID NO: 1045 - DNA, 25 bp, Homo sapiens
tattattgaatgtacccctcagcct

SEQ ID NO: 1046 - DNA, 25 bp, Homo sapiens
agcatttccttatcccaagactagt

SEQ ID NO: 1047 - DNA, 25 bp, Homo sapiens
ccaagactagtgtgctttctgctac

SEQ ID NO: 1048 - DNA, 25 bp, Homo sapiens
ctttctgctacactgctagttttca

SEQ ID NO: 1049 - DNA, 25 bp, Homo sapiens
gctacactgctagttttcagttttg

SEQ ID NO: 1050 - DNA, 25 bp, Homo sapiens
aacattaccaatttacagattcagt

SEQ ID NO: 1051 - DNA, 25 bp, Homo sapiens
ttacatttacattaatcctcactta

SEQ ID NO: 1052 - DNA, 25 bp, Homo sapiens
tgagcaagctcatttccagaaaagt

SEQ ID NO: 1053 - DNA, 25 bp, Homo sapiens
tttcagtgaagtcattttgcttcag

SEQ ID NO: 1054 - DNA, 25 bp, Homo sapiens
attatcctagttaccaagtcctatt

SEQ ID NO: 1055 - DNA, 25 bp, Homo sapiens
tatgttcgtttatcatttcagaaat

SEQ ID NO: 1056 - DNA, 25 bp, Homo sapiens
attcatgctctgctagtctatgcct

SEQ ID NO: 1057 - DNA, 25 bp, Homo sapiens
gtctatgcctgcaactccaaatgtt

SEQ ID NO: 1058 - DNA, 25 bp, Homo sapiens
cagtatttcccacctacatttctgt

SEQ ID NO: 1059 - DNA, 25 bp, Homo sapiens
tatgaccgagtctagtttttcttta

SEQ ID NO: 1060 - DNA, 25 bp, Homo sapiens
aaatacttttcatcaccaattgccc

SEQ ID NO: 1061 - DNA, 25 bp, Homo sapiens
tgcttcctcagccttgtagcaaagg

SEQ ID NO: 1062 - DNA, 25 bp, Homo sapiens
agcaaaggctacacagcagcccaca

SEQ ID NO: 1063 - DNA, 25 bp, Homo sapiens
gcccacagtccacagtctttttggg

SEQ ID NO: 1064 - DNA, 25 bp, Homo sapiens
ctgccaccttctttaagctcagttt

SEQ ID NO: 1065 - DNA, 25 bp, Homo sapiens
tttgacttactttctttgctgtagt

SEQ ID NO: 1066 - DNA, 25 bp, Homo sapiens
ttctcgtagctctgcgttgtgtgaa

SEQ ID NO: 1067 - DNA, 25 bp, Homo sapiens
agaaagcatttacctgcctgtctgt

SEQ ID NO: 1068 - DNA, 25 bp, Homo sapiens
gcatttacctgcctgtctgtaaggt

SEQ ID NO: 1069 - DNA, 25 bp, Homo sapiens
gtggaaatttcatcagtttgcaaac

SEQ ID NO: 1070 - DNA, 25 bp, Homo sapiens
aaaaagctccttccatatactgtga

SEQ ID NO: 1071 - DNA, 25 bp, Homo sapiens
gagacatttgttaagtgacatctat

SEQ ID NO: 1072 - DNA, 25 bp, Homo sapiens
gacatctattgtttatcagctttta

SEQ ID NO: 1073 - DNA, 25 bp, Homo sapiens
ggatattcctttatgagctctccat

SEQ ID NO: 1074 - DNA, 25 bp, Homo sapiens
agctctccatatccttcttgagaaa

SEQ ID NO: 1075 - DNA, 25 bp, Homo sapiens
gagagtagtctgaagattcctgtgt

SEQ ID NO: 1076 - DNA, 25 bp, Homo sapiens
aataagttctttctgcttgctgcta

SEQ ID NO: 1077 - DNA, 25 bp, Homo sapiens
tctgcttgctgctaagagtttgcta

SEQ ID NO: 1078 - DNA, 25 bp, Homo sapiens
agagcagcctgatcttacacggtgc

SEQ ID NO: 1079 - DNA, 25 bp, Homo sapiens
gtgcaaatgtgccctcatgttaaca

SEQ ID NO: 1080 - DNA, 25 bp, Homo sapiens
tcaaagggcccagttactccttacg

SEQ ID NO: 1081 - DNA, 25 bp, Homo sapiens
tactccttacgttccacaactatga

SEQ ID NO: 1082 - DNA, 25 bp, Homo sapiens
gcaaacaatattgtctcccttccag

SEQ ID NO: 1083 - DNA, 25 bp, Homo sapiens
ggttcttgaccgtgaatctggagcc

SEQ ID NO: 1084 - DNA, 25 bp, Homo sapiens
aatctggagccgtttgagttcacaa

SEQ ID NO: 1085 - DNA, 25 bp, Homo sapiens
gtctctacttggggtgacagtgctc

SEQ ID NO: 1086 - DNA, 25 bp, Homo sapiens
tgctcacgtggctcgactatagaaa

SEQ ID NO: 1087 - DNA, 25 bp, Homo sapiens
aaaactccactgactgtcgggcttt

SEQ ID NO: 1088 - DNA, 25 bp, Homo sapiens
gcttgctgtgcttcaaactactact

SEQ ID NO: 1089 - DNA, 25 bp, Homo sapiens
gaatcccttaatctacaatatcaca

SEQ ID NO: 1090 - DNA, 25 bp, Homo sapiens
tcctttctgctgtctcaggtgttat

SEQ ID NO: 1091 - DNA, 25 bp, Homo sapiens
tgagttaaatgcctggactctcccc

SEQ ID NO: 1092 - DNA, 25 bp, Homo sapiens
tcccctggctggtatcaaaacttac

SEQ ID NO: 1093 - DNA, 25 bp, Homo sapiens
aaaccagtgagatacccacctgctt

SEQ ID NO: 1094 - DNA, 25 bp, Homo sapiens
ccacctgcttgttcacatgcacagg

SEQ ID NO: 1095 - DNA, 25 bp, Homo sapiens
gttcacatgcacaggtgctctcagc

SEQ ID NO: 1096 - DNA, 25 bp, Homo sapiens
gctctcagctctgcaaagcgaatga

SEQ ID NO: 1097 - DNA, 25 bp, Homo sapiens
ggaggagcaagtccttttccaactg

SEQ ID NO: 1098 - DNA, 25 bp, Homo sapiens
ttttccaactgggtgtgcatgctaa

SEQ ID NO: 1099 - DNA, 25 bp, Homo sapiens
gatagtttagcttcagtactgtgac

SEQ ID NO: 1100 - DNA, 25 bp, Homo sapiens
gaagggtctctgatttcttgagcat

SEQ ID NO: 1101 - DNA, 25 bp, Homo sapiens
gaagccaaattctgtccaagtatta

SEQ ID NO: 1102 - DNA, 25 bp, Homo sapiens
gagagttccagttctaatagtcttt

SEQ ID NO: 1103 - DNA, 25 bp, Homo sapiens
aatggctgtattgttgctattccgt

SEQ ID NO: 1104 - DNA, 25 bp, Homo sapiens
gctattccgttgctgacatgttttt

SEQ ID NO: 1105 - DNA, 25 bp, Homo sapiens
aaagctttaacattcctgctactaa

SEQ ID NO: 1106 - DNA, 25 bp, Homo sapiens
gcggagagtgtttgccaggtttcaa

SEQ ID NO: 1107 - DNA, 25 bp, Homo sapiens
aggtttcaatgtgggctgcagcttt

SEQ ID NO: 1108 - DNA, 25 bp, Homo sapiens
ctccttctctggtttgcagtgtaat

SEQ ID NO: 1109 - DNA, 25 bp, Homo sapiens
gattatgcctcttatctacttgaga

SEQ ID NO: 1110 - DNA, 25 bp, Homo sapiens
gagagcaacatgtcttttcaatcat

SEQ ID NO: 1111 - DNA, 25 bp, Homo sapiens
ttctcttgtgcttcttggagtctgt

SEQ ID NO: 1112 - DNA, 25 bp, Homo sapiens
gtggcttggcatttctgtcatacaa

SEQ ID NO: 1113 - DNA, 25 bp, Homo sapiens
tacaagtactgcaagcgctctaagc

SEQ ID NO: 1114 - DNA, 25 bp, Homo sapiens
aacaggaattgagcccggtgtcttc

SEQ ID NO: 1115 - DNA, 25 bp, Homo sapiens
gttaccacctcaagttctatgaagc

SEQ ID NO: 1116 - DNA, 25 bp, Homo sapiens
gccaccaaacaccttagggtcttag

SEQ ID NO: 1117 - DNA, 25 bp, Homo sapiens
agactctgctgatactggacttctc

SEQ ID NO: 1118 - DNA, 25 bp, Homo sapiens
aaagtcctgctgcaccgttagagat

SEQ ID NO: 1119 - DNA, 25 bp, Homo sapiens
tctccatcttgctccagtatcagag

SEQ ID NO: 1120 - DNA, 25 bp, Homo sapiens
gatactggtctagtgggtctgtgaa

SEQ ID NO: 1121 - DNA, 25 bp, Homo sapiens
tagactgcaatatcatctcctgccc

SEQ ID NO: 1122 - DNA, 25 bp, Homo sapiens
gaagttttcagtaattgtgactttt

SEQ ID NO: 1123 - DNA, 25 bp, Homo sapiens
ggaagtacatccagtaaacaatgcc

SEQ ID NO: 1124 - DNA, 25 bp, Homo sapiens
taaacaatgccatgtacattccccc

SEQ ID NO: 1125 - DNA, 25 bp, Homo sapiens
tccccatttgctgtccagagtgtga

SEQ ID NO: 1126 - DNA, 25 bp, Homo sapiens
gagtgtgaccacagttaacggttaa

SEQ ID NO: 1127 - DNA, 25 bp, Homo sapiens
gttaacggttaatgtgcatctttta

SEQ ID NO: 1128 - DNA, 25 bp, Homo sapiens
gcatcttttatgtacttaacatgtc

SEQ ID NO: 1129 - DNA, 25 bp, Homo sapiens
gaacttccatgttagtatgtgcagc

SEQ ID NO: 1130 - DNA, 25 bp, Homo sapiens
gtgcagctgtaacacattctttttt

SEQ ID NO: 1131 - DNA, 25 bp, Homo sapiens
attctttttttagtagccacatagt

SEQ ID NO: 1132 - DNA, 25 bp, Homo sapiens
aaaatacattacccatttcctgctg

SEQ ID NO: 1133 - DNA, 25 bp, Homo sapiens
agtgcagacctgtcatctctgtctg

SEQ ID NO: 1134 - DNA, 25 bp, Homo sapiens
tctctgtctgggtttaacaccgcca

SEQ ID NO: 1135 - DNA, 25 bp, Homo sapiens
cgctcttcaccttggttcagtaact

SEQ ID NO: 1136 - DNA, 25 bp, Homo sapiens
gcaacacctacataacatgccacca

SEQ ID NO: 1137 - DNA, 25 bp, Homo sapiens
ccatctgccctcagtcagttgggag

SEQ ID NO: 1138 - DNA, 25 bp, Homo sapiens
cttgcactagcactcatttatctca

SEQ ID NO: 1139 - DNA, 25 bp, Homo sapiens
ccttctactcaaagcctcaacatca

SEQ ID NO: 1140 - DNA, 25 bp, Homo sapiens
ggcggggagatctcctgttgacagc

SEQ ID NO: 1141 - DNA, 25 bp, Homo sapiens
gcagctgtagcagttcgtacgacgg

SEQ ID NO: 1142 - DNA, 25 bp, Homo sapiens
ggatcaccggaacgaattccactcc

SEQ ID NO: 1143 - DNA, 25 bp, Homo sapiens
caagcgcatgcgactttctgaagga

SEQ ID NO: 1144 - DNA, 25 bp, Homo sapiens
taaactgatttgttgctccctatcc

SEQ ID NO: 1145 - DNA, 25 bp, Homo sapiens
accagtaactcttgtgttcaccagg

SEQ ID NO: 1146 - DNA, 25 bp, Homo sapiens
gggataggctcgttggtgacattgt

SEQ ID NO: 1147 - DNA, 25 bp, Homo sapiens
taaatggtcgatcaacttcccacaa

SEQ ID NO: 1148 - DNA, 25 bp, Homo sapiens
tgaattccacgagcctgttctgaaa

SEQ ID NO: 1149 - DNA, 25 bp, Homo sapiens
aagacaaacacgtgctcgtccttta

SEQ ID NO: 1150 - DNA, 25 bp, Homo sapiens
taatggagttcaccagcacacttgt

SEQ ID NO: 1151 - DNA, 25 bp, Homo sapiens
agcacacttgttaaccagtcctgtt

SEQ ID NO: 1152 - DNA, 25 bp, Homo sapiens
tttgctttcgtctttttttgtgcgt

SEQ ID NO: 1153 - DNA, 25 bp, Homo sapiens
atgaaaaggggctgtctggggctcc

SEQ ID NO: 1154 - DNA, 25 bp, Homo sapiens
agctccgaccatgttgctgtgtgat

SEQ ID NO: 1155 - DNA, 25 bp, Homo sapiens
ggagcaatccaagccacatatcttc

SEQ ID NO: 1156 - DNA, 25 bp, Homo sapiens
atctggtattgcattttgccttccc

SEQ ID NO: 1157 - DNA, 25 bp, Homo sapiens
cttccctgttcatacctcaaattga

SEQ ID NO: 1158 - DNA, 25 bp, Homo sapiens
aagtgacggattctgttgtggtttg

SEQ ID NO: 1159 - DNA, 25 bp, Homo sapiens
gaatgcagtaccagtgttctcttcg

SEQ ID NO: 1160 - DNA, 25 bp, Homo sapiens
gtagacctgggtcactgtaggcata

SEQ ID NO: 1161 - DNA, 25 bp, Homo sapiens
ggacttggattgcttcagatggttt

SEQ ID NO: 1162 - DNA, 25 bp, Homo sapiens
tcttttcctggggacttgtttccat

SEQ ID NO: 1163 - DNA, 25 bp, Homo sapiens
atagaggctcacagcggcataagct

SEQ ID NO: 1164 - DNA, 25 bp, Homo sapiens
tggactttgtcgccactagatgaca

SEQ ID NO: 1165 - DNA, 25 bp, Homo sapiens
ccacatctgtgtatctcaagggact

SEQ ID NO: 1166 - DNA, 25 bp, Homo sapiens
ctacctcactagtagttcacgtgat

SEQ ID NO: 1167 - DNA, 25 bp, Homo sapiens
gtagttcacgtgatgtctgacagat

SEQ ID NO: 1168 - DNA, 25 bp, Homo sapiens
tgagatactcttgtgaggtcactct

SEQ ID NO: 1169 - DNA, 25 bp, Homo sapiens
cttgtgaggtcactctaatgccctg

SEQ ID NO: 1170 - DNA, 25 bp, Homo sapiens
taagctttcatattctagccttcag

SEQ ID NO: 1171 - DNA, 25 bp, Homo sapiens
catattctagccttcagtcttgttc

SEQ ID NO: 1172 - DNA, 25 bp, Homo sapiens
cagtcttgttcttcaaccattttta

SEQ ID NO: 1173 - DNA, 25 bp, Homo sapiens
ttaggaactttcccataaggttatg

SEQ ID NO: 1174 - DNA, 25 bp, Homo sapiens
ataaggttatgttttccagcccagg

SEQ ID NO: 1175 - DNA, 25 bp, Homo sapiens
tccagcccaggcatggaggatcact

SEQ ID NO: 1176 - DNA, 25 bp, Homo sapiens
ggccacagtgaattaggattgcacc

SEQ ID NO: 1177 - DNA, 25 bp, Homo sapiens
ggggaggggactagatgggcaaggg

SEQ ID NO: 1178 - DNA, 25 bp, Homo sapiens
tgggcaaggggcagcactgcctgct

SEQ ID NO: 1179 - DNA, 25 bp, Homo sapiens
ttccttcccctgtttacagcaataa

SEQ ID NO: 1180 - DNA, 25 bp, Homo sapiens
ttacagcaataagcacgtcctcctc

SEQ ID NO: 1181 - DNA, 25 bp, Homo sapiens
actcccacttccaggattgtggttt

SEQ ID NO: 1182 - DNA, 25 bp, Homo sapiens
caagtttacaagtagacacccctgg

SEQ ID NO: 1183 - DNA, 25 bp, Homo sapiens
aaggggtgggcattggggtgccagg

SEQ ID NO: 1184 - DNA, 25 bp, Homo sapiens
ccaggcaggcatgtacagactctat

SEQ ID NO: 1185 - DNA, 25 bp, Homo sapiens
gacaggacctatgcaacgcacagac

SEQ ID NO: 1186 - DNA, 25 bp, Homo sapiens
cgcacagacacttttggagaccgta

SEQ ID NO: 1187 - DNA, 25 bp, Homo sapiens
ctttcatactctgctcttagtctaa

SEQ ID NO: 1188 - DNA, 25 bp, Homo sapiens
gactccctcaagcaagctgtggggc

SEQ ID NO: 1189 - DNA, 25 bp, Homo sapiens
ctccccactatcctgtggtgtgttg

SEQ ID NO: 1190 - DNA, 25 bp, Homo sapiens
cttcgggtcctcagatgtgtagcaa

SEQ ID NO: 1191 - DNA, 25 bp, Homo sapiens
gacactgggcagtttatgctattca

SEQ ID NO: 1192 - DNA, 25 bp, Homo sapiens
gtacatcagactgcgggttcgggct

SEQ ID NO: 1193 - DNA, 25 bp, Homo sapiens
actgccagcatgagactgctctgca

SEQ ID NO: 1194 - DNA, 25 bp, Homo sapiens
tctgcagggcaatgtcttctctaac

SEQ ID NO: 1195 - DNA, 25 bp, Homo sapiens
gtttgagcgctttaaccaggccaac

SEQ ID NO: 1196 - DNA, 25 bp, Homo sapiens
acatcaagttctctgagctcaccta

SEQ ID NO: 1197 - DNA, 25 bp, Homo sapiens
tacctcgatgcattctggcgtgact

SEQ ID NO: 1198 - DNA, 25 bp, Homo sapiens
ggcgtgactacatcaatggctcttt

SEQ ID NO: 1199 - DNA, 25 bp, Homo sapiens
gctgtggcgctggcataagtcacgc

SEQ ID NO: 1200 - DNA, 25 bp, Homo sapiens
cctgctgcaggcttctgaaggcggg

SEQ ID NO: 1201 - DNA, 25 bp, Homo sapiens
aaggcgggttggcaggtatgcccac

SEQ ID NO: 1202 - DNA, 25 bp, Homo sapiens
gtcacattttgtaggcgtggacggg

SEQ ID NO: 1203 - DNA, 25 bp, Homo sapiens
gtaggcgtggacggggtacaggctt

SEQ ID NO: 1204 - DNA, 25 bp, Homo sapiens
tctctctcattgcggactcgcagaa

SEQ ID NO: 1205 - DNA, 25 bp, Homo sapiens
cgcagaagagtcacctgattttccc

SEQ ID NO: 1206 - DNA, 25 bp, Homo sapiens
gaaaagcgagccactcttgatagct

SEQ ID NO: 1207 - DNA, 25 bp, Homo sapiens
gccactcttgatagctgaagactca

SEQ ID NO: 1208 - DNA, 25 bp, Homo sapiens
gaagactcagctatcattttaggca

SEQ ID NO: 1209 - DNA, 25 bp, Homo sapiens
ggcaaatgtgacccgacaagtaatc

SEQ ID NO: 1210 - DNA, 25 bp, Homo sapiens
gaagtgactgttgtaccatggttgt

SEQ ID NO: 1211 - DNA, 25 bp, Homo sapiens
gtaccatggttgtgcacatgcttca

SEQ ID NO: 1212 - DNA, 25 bp, Homo sapiens
gtgcacatgcttcagaatcctatgg

SEQ ID NO: 1213 - DNA, 25 bp, Homo sapiens
gaatattcctacttgcagtacatca

SEQ ID NO: 1214 - DNA, 25 bp, Homo sapiens
gaatggatggtggaccctactattc

SEQ ID NO: 1215 - DNA, 25 bp, Homo sapiens
gtggaccctactattcatgttttga

SEQ ID NO: 1216 - DNA, 25 bp, Homo sapiens
tgtgcactaccatagctacatcagt

SEQ ID NO: 1217 - DNA, 25 bp, Homo sapiens
atattttgctgtttatgatctattt

SEQ ID NO: 1218 - DNA, 25 bp, Homo sapiens
tttaaggctgtgtgaatttttctaa

SEQ ID NO: 1219 - DNA, 25 bp, Homo sapiens
tagcagtcgcgagcacatgttcata

SEQ ID NO: 1220 - DNA, 25 bp, Homo sapiens
tcccagtaggcttttaccattagca

SEQ ID NO: 1221 - DNA, 25 bp, Homo sapiens
aattcaaattgcacctcttttctta

SEQ ID NO: 1222 - DNA, 25 bp, Homo sapiens
tttgcattcttctagccagtgattg

SEQ ID NO: 1223 - DNA, 25 bp, Homo sapiens
atgctttctttgccactctaagtaa

SEQ ID NO: 1224 - DNA, 25 bp, Homo sapiens
gctggctgtttataactgcatcgca

SEQ ID NO: 1225 - DNA, 25 bp, Homo sapiens
gcatcgcacttctagttgtggcttg

SEQ ID NO: 1226 - DNA, 25 bp, Homo sapiens
tgtttcatgctaggcttttcctggc

SEQ ID NO: 1227 - DNA, 25 bp, Homo sapiens
tttcctggcagcatgtccattgcag

SEQ ID NO: 1228 - DNA, 25 bp, Homo sapiens
gaaaccaccagcattgagctaaccc

SEQ ID NO: 1229 - DNA, 25 bp, Homo sapiens
gctaacccagtacatgctaggacct

SEQ ID NO: 1230 - DNA, 25 bp, Homo sapiens
tgtcctagaggggccacttttcatt

SEQ ID NO: 1231 - DNA, 25 bp, Homo sapiens
ggccacttttcattacctgagttat

SEQ ID NO: 1232 - DNA, 25 bp, Homo sapiens
gtgatatgctgacaggctgacacgc

SEQ ID NO: 1233 - DNA, 25 bp, Homo sapiens
gctgacacgcagatggttttgtcct

SEQ ID NO: 1234 - DNA, 25 bp, Homo sapiens
ctgcgttcagtgttgaggcggctgc

SEQ ID NO: 1235 - DNA, 25 bp, Homo sapiens
gcggctgcttacaagaggcactggt

SEQ ID NO: 1236 - DNA, 25 bp, Homo sapiens
gacactcgggttgttttgtagctct

SEQ ID NO: 1237 - DNA, 25 bp, Homo sapiens
gtagctctttttcttattggctgta

SEQ ID NO: 1238 - DNA, 25 bp, Homo sapiens
tattggctgtactaacgcttgctga

SEQ ID NO: 1239 - DNA, 25 bp, Homo sapiens
aacgcttgctgaggttatctgtaat

SEQ ID NO: 1240 - DNA, 25 bp, Homo sapiens
gctttctgtgtctttcttgttcagt

SEQ ID NO: 1241 - DNA, 25 bp, Homo sapiens
gctaggtgtgtggacattgtgctaa

SEQ ID NO: 1242 - DNA, 25 bp, Homo sapiens
gtgctaaggtagtttcagtgtgtca

SEQ ID NO: 1243 - DNA, 25 bp, Homo sapiens
tgtctggaatgtggccttccacggt

SEQ ID NO: 1244 - DNA, 25 bp, Homo sapiens
gcacatccacggtgggtgagtggcc

SEQ ID NO: 1245 - DNA, 25 bp, Homo sapiens
gtccttggtgggtttagtcatctcg

SEQ ID NO: 1246 - DNA, 25 bp, Homo sapiens
gtcatctcggaagtcgtagggcagc

SEQ ID NO: 1247 - DNA, 25 bp, Homo sapiens
gaacgttccagccaggcagtggttg

SEQ ID NO: 1248 - DNA, 25 bp, Homo sapiens
aggcagtggttgttcctcataggta

SEQ ID NO: 1249 - DNA, 25 bp, Homo sapiens
taggtaggtggccttggccttcatc

SEQ ID NO: 1250 - DNA, 25 bp, Homo sapiens
tccattgcatttgtcacctagtcac

SEQ ID NO: 1251 - DNA, 25 bp, Homo sapiens
gtatatacgtgcacatttgaccttt

SEQ ID NO: 1252 - DNA, 25 bp, Homo sapiens
ttctcattcccttaactgacattat

SEQ ID NO: 1253 - DNA, 25 bp, Homo sapiens
tttagtgtcagaggccgagcacagt

SEQ ID NO: 1254 - DNA, 25 bp, Homo sapiens
ttagattcagattcctggtgcctcc

SEQ ID NO: 1255 - DNA, 25 bp, Homo sapiens
cctgggaacagactcctgtagaccc

SEQ ID NO: 1256 - DNA, 25 bp, Homo sapiens
ctagtctcctgagcctatagagccc

SEQ ID NO: 1257 - DNA, 25 bp, Homo sapiens
tagagcccccaggagactgggaccc

SEQ ID NO: 1258 - DNA, 25 bp, Homo sapiens
gggacccaaagaacttcacagcaca

SEQ ID NO: 1259 - DNA, 25 bp, Homo sapiens
tcacagcacacttaccgaatgcaga

SEQ ID NO: 1260 - DNA, 25 bp, Homo sapiens
tgcagagagcagctttcctggcttt

SEQ ID NO: 1261 - DNA, 25 bp, Homo sapiens
gcagaggctctgaagcactttcctt

SEQ ID NO: 1262 - DNA, 25 bp, Homo sapiens
tagcaacagcagctctgtacctcat

SEQ ID NO: 1263 - DNA, 25 bp, Homo sapiens
tctgttgatcccacctttgaagagg

SEQ ID NO: 1264 - DNA, 25 bp, Homo sapiens
gacacagtgctcaccttaattgcgc

SEQ ID NO: 1265 - DNA, 25 bp, Homo sapiens
gatttggtttcatcagaagcagcaa

SEQ ID NO: 1266 - DNA, 25 bp, Homo sapiens
tttttggttatggtgctattcctaa

SEQ ID NO: 1267 - DNA, 25 bp, Homo sapiens
ggtgctattcctaaggttaactttg

SEQ ID NO: 1268 - DNA, 25 bp, Homo sapiens
taactttgaatatgtgacacacaca

SEQ ID NO: 1269 - DNA, 25 bp, Homo sapiens
cacacacactcctaagtacctttaa

SEQ ID NO: 1270 - DNA, 25 bp, Homo sapiens
atattttgacagtttaggcttcatt

SEQ ID NO: 1271 - DNA, 25 bp, Homo sapiens
gaactattctgattatttggactgc

SEQ ID NO: 1272 - DNA, 25 bp, Homo sapiens
tggactgcattaattggtcactgac

SEQ ID NO: 1273 - DNA, 25 bp, Homo sapiens
taattggtcactgactggccatcca

SEQ ID NO: 1274 - DNA, 25 bp, Homo sapiens
ctggccatccaattaccattttttc

SEQ ID NO: 1275 - DNA, 25 bp, Homo sapiens
aatagttagacccttgcatacagaa

SEQ ID NO: 1276 - DNA, 25 bp, Homo sapiens
aagcttctactcctgcagtaagcac

SEQ ID NO: 1277 - DNA, 25 bp, Homo sapiens
cagtaagcacagatcgcactgcctc

SEQ ID NO: 1278 - DNA, 25 bp, Homo sapiens
gatcgcactgcctcaataacttggt

SEQ ID NO: 1279 - DNA, 25 bp, Homo sapiens
aacttggtattgagcacgtattttg

SEQ ID NO: 1280 - DNA, 25 bp, Homo sapiens
aatttccagataagacatgtcacca

SEQ ID NO: 1281 - DNA, 25 bp, Homo sapiens
caccattaattctcaacgactgctc

SEQ ID NO: 1282 - DNA, 25 bp, Homo sapiens
acgactgctctattttgttgtacgg

SEQ ID NO: 1283 - DNA, 25 bp, Homo sapiens
gtacggtaatagttatcaccttcta

SEQ ID NO: 1284 - DNA, 25 bp, Homo sapiens
tgtttattgtcttgtatcctttctc

SEQ ID NO: 1285 - DNA, 25 bp, Homo sapiens
gtatcctttctctggagtgtaagca

SEQ ID NO: 1286 - DNA, 25 bp, Homo sapiens
aatgcaacatactctcagcacctaa

SEQ ID NO: 1287 - DNA, 25 bp, Homo sapiens
tgtgctcttgatggctgggaattta

SEQ ID NO: 1288 - DNA, 25 bp, Homo sapiens
tctgctgatctgctgagaatttcaa

SEQ ID NO: 1289 - DNA, 25 bp, Homo sapiens
tctacagactgactaacatgcatta

SEQ ID NO: 1290 - DNA, 25 bp, Homo sapiens
gtaactgatagcttctgtccttatt

SEQ ID NO: 1291 - DNA, 25 bp, Homo sapiens
gcttctgtccttattagtacactta

SEQ ID NO: 1292 - DNA, 25 bp, Homo sapiens
gagactagtatttattgatccaggc

SEQ ID NO: 1293 - DNA, 25 bp, Homo sapiens
catgcttgggcttactttttcagtt

SEQ ID NO: 1294 - DNA, 25 bp, Homo sapiens
aatgccttttccatatcttaaatgt

SEQ ID NO: 1295 - DNA, 25 bp, Homo sapiens
atagacccattgtacttaagtgctg

SEQ ID NO: 1296 - DNA, 25 bp, Homo sapiens
taagtgctgatgactgttagccagt

SEQ ID NO: 1297 - DNA, 25 bp, Homo sapiens
agtttacaactttttaccatcgatg

SEQ ID NO: 1298 - DNA, 25 bp, Homo sapiens
ggtcaccggattgaaactgtctcag

SEQ ID NO: 1299 - DNA, 25 bp, Homo sapiens
gtctcaggaccttgatgatcttgcc

SEQ ID NO: 1300 - DNA, 25 bp, Homo sapiens
tctctacctggccacagttcaagcc

SEQ ID NO: 1301 - DNA, 25 bp, Homo sapiens
ctttggggactcgcttcattataga

SEQ ID NO: 1302 - DNA, 25 bp, Homo sapiens
agcagggcactcaatcagtactctt

SEQ ID NO: 1303 - DNA, 25 bp, Homo sapiens
tcttttcctatgtggaggcctcagc

SEQ ID NO: 1304 - DNA, 25 bp, Homo sapiens
gcggacattactggcatgcctgtgg

SEQ ID NO: 1305 - DNA, 25 bp, Homo sapiens
agaggtggagtccgttcttgtgggt

SEQ ID NO: 1306 - DNA, 25 bp, Homo sapiens
cctcaggggatttcgcttctgtaca

SEQ ID NO: 1307 - DNA, 25 bp, Homo sapiens
gaaagttgtgttcccgagactacag

SEQ ID NO: 1308 - DNA, 25 bp, Homo sapiens
cagggcttgcaggtgctgatgccag

SEQ ID NO: 1309 - DNA, 25 bp, Homo sapiens
tcagaaagtctgtgtcgggtcataa

SEQ ID NO: 1310 - DNA, 25 bp, Homo sapiens
gagcgagttgtaagaacccattcaa

SEQ ID NO: 1311 - DNA, 25 bp, Homo sapiens
atggcaatttttgaactagtttcta

SEQ ID NO: 1312 - DNA, 25 bp, Homo sapiens
gagctttctgggcatattgatcttt

SEQ ID NO: 1313 - DNA, 25 bp, Homo sapiens
gtgtgtgccatcaatcactttgtca

SEQ ID NO: 1314 - DNA, 25 bp, Homo sapiens
aaatgttgcacagaatcctttaaaa

SEQ ID NO: 1315 - DNA, 25 bp, Homo sapiens
gaaacactggtcatctgtacaggat

SEQ ID NO: 1316 - DNA, 25 bp, Homo sapiens
atgttcaagttttgctaataccagt

SEQ ID NO: 1317 - DNA, 25 bp, Homo sapiens
tcaggcatttgctaagtaacgatgg

SEQ ID NO: 1318 - DNA, 25 bp, Homo sapiens
tttgaagttcaatttaccatatttt

SEQ ID NO: 1319 - DNA, 25 bp, Homo sapiens
taaattgcgcattctgcacagtgaa

SEQ ID NO: 1320 - DNA, 25 bp, Homo sapiens
cagttgggatgcactacctagcgaa

SEQ ID NO: 1321 - DNA, 25 bp, Homo sapiens
acatctattgtcattccattgctat

SEQ ID NO: 1322 - DNA, 25 bp, Homo sapiens
taaaatcctagatccagttcttgtt

SEQ ID NO: 1323 - DNA, 25 bp, Homo sapiens
aaaatctgagcttctaggatccagc

SEQ ID NO: 1324 - DNA, 25 bp, Homo sapiens
cttctaggatccagctgtgtcaacc

SEQ ID NO: 1325 - DNA, 25 bp, Homo sapiens
ctgtgtcaacctttatttagcatat

SEQ ID NO: 1326 - DNA, 25 bp, Homo sapiens
atagatcaccttttacagatgctga

SEQ ID NO: 1327 - DNA, 25 bp, Homo sapiens
gattaatcttcattggtttctcaaa

SEQ ID NO: 1328 - DNA, 25 bp, Homo sapiens
taaaagggcctgtacccaaaggatg

SEQ ID NO: 1329 - DNA, 25 bp, Homo sapiens
aaacatccacgagtgctgttgcact

SEQ ID NO: 1330 - DNA, 25 bp, Homo sapiens
ctgttgcactaccatctatttgttg

SEQ ID NO: 1331 - DNA, 25 bp, Homo sapiens
gccaacgacccttacacagttagaa

SEQ ID NO: 1332 - DNA, 25 bp, Homo sapiens
tcctgatttggctatactcgaccct

SEQ ID NO: 1333 - DNA, 25 bp, Homo sapiens
ttcagtggtgtgcggagtcctggca

SEQ ID NO: 1334 - DNA, 25 bp, Homo sapiens
ctacttcaccctgttcatcgtgatg

SEQ ID NO: 1335 - DNA, 25 bp, Homo sapiens
tgatgatgttatatgccccagcagc

SEQ ID NO: 1336 - DNA, 25 bp, Homo sapiens
ggcctgtcctgataagcgctatgcc

SEQ ID NO: 1337 - DNA, 25 bp, Homo sapiens
ctatgccatggtcctgtttcgaatc

SEQ ID NO: 1338 - DNA, 25 bp, Homo sapiens
gtattttacatcctctggttgccat

SEQ ID NO: 1339 - DNA, 25 bp, Homo sapiens
ggttgccatatatcatctacttctt

SEQ ID NO: 1340 - DNA, 25 bp, Homo sapiens
gactaaagcgcctctcaggggctat

SEQ ID NO: 1341 - DNA, 25 bp, Homo sapiens
caggggctatgtgtacttcttgtgc

SEQ ID NO: 1342 - DNA, 25 bp, Homo sapiens
tggcagccacatccaagactggagc

SEQ ID NO: 1343 - DNA, 25 bp, Homo sapiens
ccaagactggagcagcaggctggcc

SEQ ID NO: 1344 - DNA, 25 bp, Homo sapiens
agagagagctcacagctgaagctct

SEQ ID NO: 1345 - DNA, 25 bp, Homo sapiens
agctcacagctgaagctcttggagg

SEQ ID NO: 1346 - DNA, 25 bp, Homo sapiens
gaccaggagcatggtgaagccaagt

SEQ ID NO: 1347 - DNA, 25 bp, Homo sapiens
ccaagtggcagatgggagccaacct

SEQ ID NO: 1348 - DNA, 25 bp, Homo sapiens
tttgccctgcatcctgtcatttctg

SEQ ID NO: 1349 - DNA, 25 bp, Homo sapiens
gttcttgtccctcatacatctttgg

SEQ ID NO: 1350 - DNA, 25 bp, Homo sapiens
ttgtccctcatacatctttggagaa

SEQ ID NO: 1351 - DNA, 25 bp, Homo sapiens
ccctcatacatctttggagaaccgg

SEQ ID NO: 1352 - DNA, 25 bp, Homo sapiens
tcatacatctttggagaaccgggct

SEQ ID NO: 1353 - DNA, 25 bp, Homo sapiens
tgccttatggctctagtgtgtgacc

SEQ ID NO: 1354 - DNA, 25 bp, Homo sapiens
cttatggctctagtgtgtgacctac

SEQ ID NO: 1355 - DNA, 25 bp, Homo sapiens
atggctctagtgtgtgacctacaga

SEQ ID NO: 1356 - DNA, 25 bp, Homo sapiens
ctctagtgtgtgacctacagagcat

SEQ ID NO: 1357 - DNA, 25 bp, Homo sapiens
tgtgacctacagagcatgctccaca

SEQ ID NO: 1358 - DNA, 25 bp, Homo sapiens
tccgagctaaaatcccagggaccgg

SEQ ID NO: 1359 - DNA, 25 bp, Homo sapiens
ttacctgagcgaccaggactacatt

SEQ ID NO: 1360 - DNA, 25 bp, Homo sapiens
gcctgctgggacttgtagttgccta

SEQ ID NO: 1361 - DNA, 25 bp, Homo sapiens
tgctgggacttgtagttgcctagac

SEQ ID NO: 1362 - DNA, 25 bp, Homo sapiens
tgggacttgtagttgcctagacagg

SEQ ID NO: 1363 - DNA, 25 bp, Homo sapiens
tgtagttgcctagacagggcaccac

SEQ ID NO: 1364 - DNA, 25 bp, Homo sapiens
gtagttgcctagacagggcaccacc

SEQ ID NO: 1365 - DNA, 25 bp, Homo sapiens
aggcgttggtgtctcctggatgcta

SEQ ID NO: 1366 - DNA, 25 bp, Homo sapiens
ggcgttggtgtctcctggatgctac

SEQ ID NO: 1367 - DNA, 25 bp, Homo sapiens
gcgttggtgtctcctggatgctact

SEQ ID NO: 1368 - DNA, 25 bp, Homo sapiens
cgttggtgtctcctggatgctacta

SEQ ID NO: 1369 - DNA, 25 bp, Homo sapiens
gggaggcctgagcttggatttacac

SEQ ID NO: 1370 - DNA, 25 bp, Homo sapiens
ggaggcctgagcttggatttacact

SEQ ID NO: 1371 - DNA, 25 bp, Homo sapiens
ggcctgagcttggatttacactgta

SEQ ID NO: 1372 - DNA, 25 bp, Homo sapiens
ctgagcttggatttacactgtaata

SEQ ID NO: 1373 - DNA, 25 bp, Homo sapiens
cttggatttacactgtaataaagac

SEQ ID NO: 1374 - DNA, 25 bp, Homo sapiens
agaatggtgtttacagtgctgcaaa

SEQ ID NO: 1375 - DNA, 25 bp, Homo sapiens
cagtgctgcaaattacaccaatggg

SEQ ID NO: 1376 - DNA, 25 bp, Homo sapiens
gaagtaattttgtgtctgctggtat

SEQ ID NO: 1377 - DNA, 25 bp, Homo sapiens
aggactggtaatccaacagggactt

SEQ ID NO: 1378 - DNA, 25 bp, Homo sapiens
gaatggttatgatagcactcagcaa

SEQ ID NO: 1379 - DNA, 25 bp, Homo sapiens
tgcatatcctgctactgcagctgca

SEQ ID NO: 1380 - DNA, 25 bp, Homo sapiens
gcagctgcacctatgattggttatc

SEQ ID NO: 1381 - DNA, 25 bp, Homo sapiens
tccaatgccaacaggatattcccaa

SEQ ID NO: 1382 - DNA, 25 bp, Homo sapiens
gtctgtttttcataattgctcttta

SEQ ID NO: 1383 - DNA, 25 bp, Homo sapiens
tatggtgcactttttcgctatttaa

SEQ ID NO: 1384 - DNA, 25 bp, Homo sapiens
agttggatatttctctacattcctg

SEQ ID NO: 1385 - DNA, 25 bp, Homo sapiens
agaatggtgtttacagtgctgcaaa

SEQ ID NO: 1386 - DNA, 25 bp, Homo sapiens
cagtgctgcaaattacaccaatggg

SEQ ID NO: 1387 - DNA, 25 bp, Homo sapiens
gaagtaattttgtgtctgctggtat

SEQ ID NO: 1388 - DNA, 25 bp, Homo sapiens
aggactggtaatccaacagggactt

SEQ ID NO: 1389 - DNA, 25 bp, Homo sapiens
gaatggttatgatagcactcagcaa

SEQ ID NO: 1390 - DNA, 25 bp, Homo sapiens
tgcatatcctgctactgcagctgca

SEQ ID NO: 1391 - DNA, 25 bp, Homo sapiens
gcagctgcacctatgattggttatc

SEQ ID NO: 1392 - DNA, 25 bp, Homo sapiens
tccaatgccaacaggatattcccaa

SEQ ID NO: 1393 - DNA, 25 bp, Homo sapiens
gtctgtttttcataattgctcttta

SEQ ID NO: 1394 - DNA, 25 bp, Homo sapiens
tatggtgcactttttcgctatttaa

SEQ ID NO: 1395 - DNA, 25 bp, Homo sapiens
agttggatatttctctacattcctg

SEQ ID NO: 1396 - DNA, 25 bp, Homo sapiens
gatatgagtctgcatggcctcagga

SEQ ID NO: 1397 - DNA, 25 bp, Homo sapiens
gattttaggttgtctgcactctagc

SEQ ID NO: 1398 - DNA, 25 bp, Homo sapiens
gcactctagcttttttgtcgttttc

SEQ ID NO: 1399 - DNA, 25 bp, Homo sapiens
atacatcatatcttaatttccactg

SEQ ID NO: 1400 - DNA, 25 bp, Homo sapiens
tctacacggccggggtttcaacaag

SEQ ID NO: 1401 - DNA, 25 bp, Homo sapiens
acaaggtactgatgtcttctgccct

SEQ ID NO: 1402 - DNA, 25 bp, Homo sapiens
tgcccttgcctcttcgacaggcaag

SEQ ID NO: 1403 - DNA, 25 bp, Homo sapiens
attctttaggcacacaaattcacat

SEQ ID NO: 1404 - DNA, 25 bp, Homo sapiens
cattatacttcctgatctgtgattg

SEQ ID NO: 1405 - DNA, 25 bp, Homo sapiens
tcgtaactagtatgtctgtcccacc

SEQ ID NO: 1406 - DNA, 25 bp, Homo sapiens
tatgtctgtcccacctttaaaaagt

SEQ ID NO: 1407 - DNA, 25 bp, Homo sapiens
aaatcactctcagtgcttcttacta

SEQ ID NO: 1408 - DNA, 25 bp, Homo sapiens
gcagtaaaaactgttctctattaga

SEQ ID NO: 1409 - DNA, 25 bp, Homo sapiens
atgtacctgatgtacctgatgctat

SEQ ID NO: 1410 - DNA, 25 bp, Homo sapiens
atctatatggaattgcttaccaaag

SEQ ID NO: 1411 - DNA, 25 bp, Homo sapiens
tagtgcgatgtttcaggaggctgga

SEQ ID NO: 1412 - DNA, 25 bp, Homo sapiens
agcccactgagaagtcaaacatttc

SEQ ID NO: 1413 - DNA, 25 bp, Homo sapiens
gtggcatgtgctgtgaccatttata

SEQ ID NO: 1414 - DNA, 25 bp, Homo sapiens
tttacaataggtgcttattctcaaa

SEQ ID NO: 1415 - DNA, 25 bp, Homo sapiens
aggtgcttattctcaaagcaggaat

SEQ ID NO: 1416 - DNA, 25 bp, Homo sapiens
gcaggaattggtggcagattttaca

SEQ ID NO: 1417 - DNA, 25 bp, Homo sapiens
cttctctttgacaattcctagataa

SEQ ID NO: 1418 - DNA, 25 bp, Homo sapiens
tagcagccctatctttgggcctttg

SEQ ID NO: 1419 - DNA, 25 bp, Homo sapiens
gggcctttggtggacatttgatcgt

SEQ ID NO: 1420 - DNA, 25 bp, Homo sapiens
tgatcgttccagaagtggccttggg

SEQ ID NO: 1421 - DNA, 25 bp, Homo sapiens
ggctggggatcaccatagcttttct

SEQ ID NO: 1422 - DNA, 25 bp, Homo sapiens
tagctacgctgatcacgcagtttct

SEQ ID NO: 1423 - DNA, 25 bp, Homo sapiens
ttcctctatattcgttcttggctcc

SEQ ID NO: 1424 - DNA, 25 bp, Homo sapiens
ttttctcaggaggcgtcacggtggg

SEQ ID NO: 1425 - DNA, 25 bp, Homo sapiens
gttcctgaaaagccccatagtgatt

SEQ ID NO: 1426 - DNA, 25 bp, Homo sapiens
gggctgactgtacaaatgactcctg

SEQ ID NO: 1427 - DNA, 25 bp, Homo sapiens
gatgacttaccctgaagtcttccct

SEQ ID NO: 1428 - DNA, 25 bp, Homo sapiens
cttcccaagtattcgatttcattca

SEQ ID NO: 1429 - DNA, 25 bp, Homo sapiens
tcccacacaagacccaagtagctgc

SEQ ID NO: 1430 - DNA, 25 bp, Homo sapiens
ccaagtagctgctaccttctttggc

SEQ ID NO: 1431 - DNA, 25 bp, Homo sapiens
tctaccagacccttctggtgccaga

SEQ ID NO: 1432 - DNA, 25 bp, Homo sapiens
tcattcctgttctttcttacacaag

SEQ ID NO: 1433 - DNA, 25 bp, Homo sapiens
gactcgggccttagaactttgcata

SEQ ID NO: 1434 - DNA, 25 bp, Homo sapiens
atagcagctgctactagctctttga

SEQ ID NO: 1435 - DNA, 25 bp, Homo sapiens
atacattccgaggggctcagttctg

SEQ ID NO: 1436 - DNA, 25 bp, Homo sapiens
gcttctcactcatcactaacaaggt

SEQ ID NO: 1437 - DNA, 25 bp, Homo sapiens
gaacagtttgtctccattcttatgg

SEQ ID NO: 1438 - DNA, 25 bp, Homo sapiens
tccattcttatggccagcattccac

SEQ ID NO: 1439 - DNA, 25 bp, Homo sapiens
actccctgacaaagccagttgacct

SEQ ID NO: 1440 - DNA, 25 bp, Homo sapiens
ttcagactctatctttgcttgttca

SEQ ID NO: 1441 - DNA, 25 bp, Homo sapiens
tgggtctctttatcgtggtctgaca

SEQ ID NO: 1442 - DNA, 25 bp, Homo sapiens
cgtggtctgacaactcatctagtga

SEQ ID NO: 1443 - DNA, 25 bp, Homo sapiens
gaattggtggtttacctactcaatg

SEQ ID NO: 1444 - DNA, 25 bp, Homo sapiens
gcagcacgaggactgctgtactgca

SEQ ID NO: 1445 - DNA, 25 bp, Homo sapiens
atcacaccacattacttggcctttc

SEQ ID NO: 1446 - DNA, 25 bp, Homo sapiens
gggcatgtctgcttcatatgctggt

SEQ ID NO: 1447 - DNA, 25 bp, Homo sapiens
atcagagactgttatccattttgtt

SEQ ID NO: 1448 - DNA, 25 bp, Homo sapiens
gtgggaatgatgctagctgctgcca

SEQ ID NO: 1449 - DNA, 25 bp, Homo sapiens
gctgccacctcaaaaacttgtgcca

SEQ ID NO: 1450 - DNA, 25 bp, Homo sapiens
gccacaactatagcatatccacatg

SEQ ID NO: 1451 - DNA, 25 bp, Homo sapiens
acctgctctccacaataaatcacaa

SEQ ID NO: 1452 - DNA, 25 bp, Homo sapiens
acagctgtcagaacctcgagagcag

SEQ ID NO: 1453 - DNA, 25 bp, Homo sapiens
actcagagctctggaccgaaagcag

SEQ ID NO: 1454 - DNA, 25 bp, Homo sapiens
attaccatcgattcagtgcctggat

SEQ ID NO: 1455 - DNA, 25 bp, Homo sapiens
gcttacttgtttaatggcagccaca

SEQ ID NO: 1456 - DNA, 25 bp, Homo sapiens
ggcagccacatgcacgaagatgcta

SEQ ID NO: 1457 - DNA, 25 bp, Homo sapiens
gaattccaaatcctcaacttttgag

SEQ ID NO: 1458 - DNA, 25 bp, Homo sapiens
acttttgaggtttcggctctccaat

SEQ ID NO: 1459 - DNA, 25 bp, Homo sapiens
ttcggctctccaatttaactctttg

SEQ ID NO: 1460 - DNA, 25 bp, Homo sapiens
agttcaaggttcactccctatatgt

SEQ ID NO: 1461 - DNA, 25 bp, Homo sapiens
gattaacatacccgtctatgcctaa

SEQ ID NO: 1462 - DNA, 25 bp, Homo sapiens
gagcagtaaatcaatggaacatccc

SEQ ID NO: 1463 - DNA, 25 bp, Homo sapiens
acaaattggacttgttcaactgctg

SEQ ID NO: 1464 - DNA, 25 bp, Homo sapiens
cagccccaacttaaaattcttacat

SEQ ID NO: 1465 - DNA, 25 bp, Homo sapiens
acagaccaacctggcattacagttg

SEQ ID NO: 1466 - DNA, 25 bp, Homo sapiens
ttggcctctccttgaggtgggcaca

SEQ ID NO: 1467 - DNA, 25 bp, Homo sapiens
gccaggggtggccatgtaagtccca

SEQ ID NO: 1468 - DNA, 25 bp, Homo sapiens
gctacccgagtttagtaacagtgca

SEQ ID NO: 1469 - DNA, 25 bp, Homo sapiens
aacagtgcagattccacgttcttgt

SEQ ID NO: 1470 - DNA, 25 bp, Homo sapiens
cgttcttgttccgatactctgagaa

SEQ ID NO: 1471 - DNA, 25 bp, Homo sapiens
gatgttgatgtacttacagacacaa

SEQ ID NO: 1472 - DNA, 25 bp, Homo sapiens
gacacaagaacaatctttgctataa

SEQ ID NO: 1473 - DNA, 25 bp, Homo sapiens
ctcttgtcaaatctgtgtcggctgc

SEQ ID NO: 1474 - DNA, 25 bp, Homo sapiens
ccctgatccttccattatcaagttt

SEQ ID NO: 1475 - DNA, 25 bp, Homo sapiens
actgatgtaacctcaaagcctctca

SEQ ID NO: 1476 - DNA, 25 bp, Homo sapiens
tcaccattcctcttggcttggaaag

SEQ ID NO: 1477 - DNA, 25 bp, Homo sapiens
acaagcgattgtccatctgttgcct

SEQ ID NO: 1478 - DNA, 25 bp, Homo sapiens
cctgctttagccatctgtgggaaac

SEQ ID NO: 1479 - DNA, 25 bp, Homo sapiens
gacacctctgcaaaatgtgcctcaa

SEQ ID NO: 1480 - DNA, 25 bp, Homo sapiens
gtgcctcaagtccatttcttgggat

SEQ ID NO: 1481 - DNA, 25 bp, Homo sapiens
catttcttgggatcgctcgtttggt

SEQ ID NO: 1482 - DNA, 25 bp, Homo sapiens
gtttggtgcactctcgtgggagaca

SEQ ID NO: 1483 - DNA, 25 bp, Homo sapiens
aacatatacttgtgccttattttca

SEQ ID NO: 1484 - DNA, 25 bp, Homo sapiens
gtttgatatttaccacagcgctgtg

SEQ ID NO: 1485 - DNA, 25 bp, Homo sapiens
gcgctgtgcctttctacagtagaac

SEQ ID NO: 1486 - DNA, 25 bp, Homo sapiens
ggttttattgcccatagtcatttag

SEQ ID NO: 1487 - DNA, 25 bp, Homo sapiens
atatttctttcttagttgttggcac

SEQ ID NO: 1488 - DNA, 25 bp, Homo sapiens
gttgttggcactcttaggtcttagt

SEQ ID NO: 1489 - DNA, 25 bp, Homo sapiens
gtgtgtgtgtagtttatcctctctc

SEQ ID NO: 1490 - DNA, 25 bp, Homo sapiens
gattgactgatacctcattctgttt

SEQ ID NO: 1491 - DNA, 25 bp, Homo sapiens
aatttctgtgcaaccttactatgtg

SEQ ID NO: 1492 - DNA, 25 bp, Homo sapiens
gtgtgcttttgttttcggatagact

SEQ ID NO: 1493 - DNA, 25 bp, Homo sapiens
atttctttagttctgcacttttcca

SEQ ID NO: 1494 - DNA, 25 bp, Homo sapiens
cacttttccacattatactccatat

SEQ ID NO: 1495 - DNA, 25 bp, Homo sapiens
tggcagtggttctggtactaaaaat

SEQ ID NO: 1496 - DNA, 25 bp, Homo sapiens
gttctggtactaaaaattgtggttg

SEQ ID NO: 1497 - DNA, 25 bp, Homo sapiens
ttttctgtttacgtaacctgcttag

SEQ ID NO: 1498 - DNA, 25 bp, Homo sapiens
acgtaacctgcttagtattgacact

SEQ ID NO: 1499 - DNA, 25 bp, Homo sapiens
aacctgcttagtattgacactctct

SEQ ID NO: 1500 - DNA, 25 bp, Homo sapiens
gcttagtattgacactctctaccaa

SEQ ID NO: 1501 - DNA, 25 bp, Homo sapiens
gacactctctaccaagagggtcttc

SEQ ID NO: 1502 - DNA, 25 bp, Homo sapiens
ctctaccaagagggtcttcctaaga

SEQ ID NO: 1503 - DNA, 25 bp, Homo sapiens
cttcctaagaagagtgctgtcatta

SEQ ID NO: 1504 - DNA, 25 bp, Homo sapiens
gagtgctgtcattatttcctcttat

SEQ ID NO: 1505 - DNA, 25 bp, Homo sapiens
tttcctcttatcaacaacttgtgac

SEQ ID NO: 1506 - DNA, 25 bp, Homo sapiens
gagatagctcgcattcagactacct

SEQ ID NO: 1507 - DNA, 25 bp, Homo sapiens
ctcgcattcagactacctactaaca

SEQ ID NO: 1508 - DNA, 25 bp, Homo sapiens
tcagctggaccaactaatcttcgaa

SEQ ID NO: 1509 - DNA, 25 bp, Homo sapiens
aaattcagattggactctatcatat

SEQ ID NO: 1510 - DNA, 25 bp, Homo sapiens
atcatatgtgtcaaatccaagctta

SEQ ID NO: 1511 - DNA, 25 bp, Homo sapiens
gtgtggttcatctgatcgactacta

SEQ ID NO: 1512 - DNA, 25 bp, Homo sapiens
tcgactactatgttcagatgtgcaa

SEQ ID NO: 1513 - DNA, 25 bp, Homo sapiens
taagcggacaggtccagaagccccc

SEQ ID NO: 1514 - DNA, 25 bp, Homo sapiens
actgttcacctttatctgaccaaac

SEQ ID NO: 1515 - DNA, 25 bp, Homo sapiens
ctgaccaaaccgctctacacgtcag

SEQ ID NO: 1516 - DNA, 25 bp, Homo sapiens
ttaacaaatgtaccggtgccatctg

SEQ ID NO: 1517 - DNA, 25 bp, Homo sapiens
aggatccggagctggtgctgataac

SEQ ID NO: 1518 - DNA, 25 bp, Homo sapiens
tgctgataacagcggaatcccccgt

SEQ ID NO: 1519 - DNA, 25 bp, Homo sapiens
ttggtcctggaacagcgctactgat

SEQ ID NO: 1520 - DNA, 25 bp, Homo sapiens
gtcctggaacagcgctactgatcac

SEQ ID NO: 1521 - DNA, 25 bp, Homo sapiens
ggaacagcgctactgatcaccaagt

SEQ ID NO: 1522 - DNA, 25 bp, Homo sapiens
tcaccaagtagccacaaaatataat

SEQ ID NO: 1523 - DNA, 25 bp, Homo sapiens
tataataaaccctcagcacttgctc

SEQ ID NO: 1524 - DNA, 25 bp, Homo sapiens
ataaaccctcagcacttgctcagta

SEQ ID NO: 1525 - DNA, 25 bp, Homo sapiens
aaccctcagcacttgctcagtagtt

SEQ ID NO: 1526 - DNA, 25 bp, Homo sapiens
cagcacttgctcagtagttttgtga

SEQ ID NO: 1527 - DNA, 25 bp, Homo sapiens
gtagttttgtgaaagtctcaagtaa

SEQ ID NO: 1528 - DNA, 25 bp, Homo sapiens
gatttaacattgttgggccatttaa

SEQ ID NO: 1529 - DNA, 25 bp, Homo sapiens
aaatgtgcatattggagcagaacat

SEQ ID NO: 1530 - DNA, 25 bp, Homo sapiens
atctgtttccattttagtcacagaa

SEQ ID NO: 1531 - DNA, 25 bp, Homo sapiens
acaatgctttctacctgaaatgtgt

SEQ ID NO: 1532 - DNA, 25 bp, Homo sapiens
cctctcagtccttgttcttttgaag

SEQ ID NO: 1533 - DNA, 25 bp, Homo sapiens
gtccttgttcttttgaagcttgtgc

SEQ ID NO: 1534 - DNA, 25 bp, Homo sapiens
gcttgtgctgaggttttagcttttc

SEQ ID NO: 1535 - DNA, 25 bp, Homo sapiens
gtgctgaggttttagcttttctatg

SEQ ID NO: 1536 - DNA, 25 bp, Homo sapiens
gccgctgctttgaaagagaacctag

SEQ ID NO: 1537 - DNA, 25 bp, Homo sapiens
gagaacctagattctatagttgtat

SEQ ID NO: 1538 - DNA, 25 bp, Homo sapiens
tattattgttgtttcatactttaaa

SEQ ID NO: 1539 - DNA, 25 bp, Homo sapiens
ggtccctttttccgaggaagagctg

SEQ ID NO: 1540 - DNA, 25 bp, Homo sapiens
attttttcaccagtacgctctgtgc

SEQ ID NO: 1541 - DNA, 25 bp, Homo sapiens
ctccttggccgtctactggaaaaat

SEQ ID NO: 1542 - DNA, 25 bp, Homo sapiens
actggaaaaatcgagcctctcccac

SEQ ID NO: 1543 - DNA, 25 bp, Homo sapiens
ccaccctcagtcgcatagacttatg

SEQ ID NO: 1544 - DNA, 25 bp, Homo sapiens
gaattagcgtttaatccacttcctt

SEQ ID NO: 1545 - DNA, 25 bp, Homo sapiens
tattgggcactcggttatcttttaa

SEQ ID NO: 1546 - DNA, 25 bp, Homo sapiens
ttccgtttggtagactccttccaat

SEQ ID NO: 1547 - DNA, 25 bp, Homo sapiens
ggtagactccttccaatgaaatctc

SEQ ID NO: 1548 - DNA, 25 bp, Homo sapiens
cccgggccattgccagaagacgtct

SEQ ID NO: 1549 - DNA, 25 bp, Homo sapiens
gccagaagacgtcttctcggggcgc

SEQ ID NO: 1550 - DNA, 25 bp, Homo sapiens
atgcttgcccaacacactgtgaaat

SEQ ID NO: 1551 - DNA, 25 bp, Homo sapiens
atgcagcatcttcattctttctgag

SEQ ID NO: 1552 - DNA, 25 bp, Homo sapiens
gatggttttctttacatgaacaaat

SEQ ID NO: 1553 - DNA, 25 bp, Homo sapiens
gagatcctagatccataacgtagct

SEQ ID NO: 1554 - DNA, 25 bp, Homo sapiens
aaggcatctaagagtttgctgttga

SEQ ID NO: 1555 - DNA, 25 bp, Homo sapiens
tgctgttgataatcttgctgaccaa

SEQ ID NO: 1556 - DNA, 25 bp, Homo sapiens
gtaacacaggttatatgccatcaca

SEQ ID NO: 1557 - DNA, 25 bp, Homo sapiens
atgccatcacaaatacaatgctcat

SEQ ID NO: 1558 - DNA, 25 bp, Homo sapiens
agagtcaatgaacctgtgtccagaa

SEQ ID NO: 1559 - DNA, 25 bp, Homo sapiens
agaggtcttaactttgcatttataa

SEQ ID NO: 1560 - DNA, 25 bp, Homo sapiens
tcatttgcagtctttgtatttaaaa

SEQ ID NO: 1561 - DNA, 25 bp, Homo sapiens
atgatgaggtggtctaccctacctg

SEQ ID NO: 1562 - DNA, 25 bp, Homo sapiens
ccctacctggctccatgaagatgcc

SEQ ID NO: 1563 - DNA, 25 bp, Homo sapiens
cagggaggcgagcacgccatcttga

SEQ ID NO: 1564 - DNA, 25 bp, Homo sapiens
gccatcttgagacatccttttttta

SEQ ID NO: 1565 - DNA, 25 bp, Homo sapiens
ttaaggaaatcgactgggcccagct

SEQ ID NO: 1566 - DNA, 25 bp, Homo sapiens
gaaccatcgccaaatagaaccgcct

SEQ ID NO: 1567 - DNA, 25 bp, Homo sapiens
tcagacccagaatcaaatcccgaga

SEQ ID NO: 1568 - DNA, 25 bp, Homo sapiens
agaaacttttcctatgtgtctccag

SEQ ID NO: 1569 - DNA, 25 bp, Homo sapiens
gtgtctccagaattgcaaccatagc

SEQ ID NO: 1570 - DNA, 25 bp, Homo sapiens
ccaggaatttcctctatcggacctt

SEQ ID NO: 1571 - DNA, 25 bp, Homo sapiens
cttcccagcatcagccttagaacaa

SEQ ID NO: 1572 - DNA, 25 bp, Homo sapiens
ctctgatccctcaatttggtctgtt

SEQ ID NO: 1573 - DNA, 25 bp, Homo sapiens
atagaacgccaaactgctctcagta

SEQ ID NO: 1574 - DNA, 25 bp, Homo sapiens
tagattaccaggatgtcccagacac

SEQ ID NO: 1575 - DNA, 25 bp, Homo sapiens
tcccagacactttaaccctgtgtgt

SEQ ID NO: 1576 - DNA, 25 bp, Homo sapiens
ccctgtgtgtggcagtgacatgtcc

SEQ ID NO: 1577 - DNA, 25 bp, Homo sapiens
gtgacatgtccacttatgccaatga

SEQ ID NO: 1578 - DNA, 25 bp, Homo sapiens
cattcgaaatggaccctgctgatgg

SEQ ID NO: 1579 - DNA, 25 bp, Homo sapiens
ggcgcaggtaacagaccgcaggggc

SEQ ID NO: 1580 - DNA, 25 bp, Homo sapiens
agaatccttgtttcttggcttttgc

SEQ ID NO: 1581 - DNA, 25 bp, Homo sapiens
tcttggcttttgctcctggagttaa

SEQ ID NO: 1582 - DNA, 25 bp, Homo sapiens
gagttaagcttactgcccaggtgac

SEQ ID NO: 1583 - DNA, 25 bp, Homo sapiens
gatggccgtgtttcggaatgtcctc

SEQ ID NO: 1584 - DNA, 25 bp, Homo sapiens
tcctcacacctaccaaaacaggcga

SEQ ID NO: 1585 - DNA, 25 bp, Homo sapiens
aaacaggcgacctttcagcagagag

SEQ ID NO: 1586 - DNA, 25 bp, Homo sapiens
agatggatttggggctactctctcc

SEQ ID NO: 1587 - DNA, 25 bp, Homo sapiens
ctccgcaggctcaggtcgaagattc

SEQ ID NO: 1588 - DNA, 25 bp, Homo sapiens
tagttttaaggacttcatccctcca

SEQ ID NO: 1589 - DNA, 25 bp, Homo sapiens
ccacctatccctaacaggctgtaga

SEQ ID NO: 1590 - DNA, 25 bp, Homo sapiens
ttatcaactgctgcttcaccagact

SEQ ID NO: 1591 - DNA, 25 bp, Homo sapiens
tttctctagaagccgtctgcgttta

SEQ ID NO: 1592 - DNA, 25 bp, Homo sapiens
ggagcattgatctgcatccaaggcc

SEQ ID NO: 1593 - DNA, 25 bp, Homo sapiens
ggccggcttgagtgaattgtgtacc

SEQ ID NO: 1594 - DNA, 25 bp, Homo sapiens
agcgtgttcccaatagtgtactctg

SEQ ID NO: 1595 - DNA, 25 bp, Homo sapiens
tactctggctgttgcgttttccagc

SEQ ID NO: 1596 - DNA, 25 bp, Homo sapiens
gccccagaaccgtatcattttttca

SEQ ID NO: 1597 - DNA, 25 bp, Homo sapiens
gaggaacacgtcaggagaggccagc

SEQ ID NO: 1598 - DNA, 25 bp, Homo sapiens
ggacactccactctgtaatgggcac

SEQ ID NO: 1599 - DNA, 25 bp, Homo sapiens
gatggatgtcctctgggcagggacc

SEQ ID NO: 1600 - DNA, 25 bp, Homo sapiens
acccccatggtgactatgccaggag

SEQ ID NO: 1601 - DNA, 25 bp, Homo sapiens
aggagagactcttgcttctcgagtt

SEQ ID NO: 1602 - DNA, 25 bp, Homo sapiens
atcccagctcacttgcttaggttgt

SEQ ID NO: 1603 - DNA, 25 bp, Homo sapiens
gagcggctctatctacagatgtggg

SEQ ID NO: 1604 - DNA, 25 bp, Homo sapiens
tgcagctggcaacaaacctgaccac

SEQ ID NO: 1605 - DNA, 25 bp, Homo sapiens
tcagtcttctggatttttttttctt

SEQ ID NO: 1606 - DNA, 25 bp, Homo sapiens
taagctaaaatgttactccctgttt

SEQ ID NO: 1607 - DNA, 25 bp, Homo sapiens
tactccctgttttagtttctgaact

SEQ ID NO: 1608 - DNA, 25 bp, Homo sapiens
gggactttgctggtgtagtcttttt

SEQ ID NO: 1609 - DNA, 25 bp, Homo sapiens
accacttgagcctatatcagtcgtt

SEQ ID NO: 1610 - DNA, 25 bp, Homo sapiens
atcagtcgttttagtgtctgaccta

SEQ ID NO: 1611 - DNA, 25 bp, Homo sapiens
gtctgacctaatatttggagctatc

SEQ ID NO: 1612 - DNA, 25 bp, Homo sapiens
ggagctatcagtgctttgttgattt

SEQ ID NO: 1613 - DNA, 25 bp, Homo sapiens
agattttttctggtccatttcccat

SEQ ID NO: 1614 - DNA, 25 bp, Homo sapiens
tcacccttaaaattctcctgtaact

SEQ ID NO: 1615 - DNA, 25 bp, Homo sapiens
aagcctgattcaaaacatcctaggg

SEQ ID NO: 1616 - DNA, 25 bp, Homo sapiens
ggagagctattccgtgtacgtgtac

SEQ ID NO: 1617 - DNA, 25 bp, Homo sapiens
caaggtgctgaagcaggtccacccc

SEQ ID NO: 1618 - DNA, 25 bp, Homo sapiens
gcctgaaccagctaagtcagctccc

SEQ ID NO: 1619 - DNA, 25 bp, Homo sapiens
catctcgtccaaggctatggggatt

SEQ ID NO: 1620 - DNA, 25 bp, Homo sapiens
gattatgaactccttcgtcaacgac

SEQ ID NO: 1621 - DNA, 25 bp, Homo sapiens
ttttcgagcgcattgcaggcgaggc

SEQ ID NO: 1622 - DNA, 25 bp, Homo sapiens
tcccgcctggcgcattataacaagc

SEQ ID NO: 1623 - DNA, 25 bp, Homo sapiens
ttataacaagcgctcgaccatcact

SEQ ID NO: 1624 - DNA, 25 bp, Homo sapiens
ccagggagatccaaacggctgtgcg

SEQ ID NO: 1625 - DNA, 25 bp, Homo sapiens
aaacacgcggtgtcggagggcacca

SEQ ID NO: 1626 - DNA, 25 bp, Homo sapiens
gaagggctccaagaaggcggtgacc

SEQ ID NO: 1627 - DNA, 25 bp, Homo sapiens
aagcgcagccgcaaggagagctact

SEQ ID NO: 1628 - DNA, 25 bp, Homo sapiens
gagagctactccgtatacgtgtaca

SEQ ID NO: 1629 - DNA, 25 bp, Homo sapiens
atgcctgagccagcgaaatccgctc

SEQ ID NO: 1630 - DNA, 25 bp, Homo sapiens
gcatctcctctaaagccatggggat

SEQ ID NO: 1631 - DNA, 25 bp, Homo sapiens
tgtcaacgacatcttcgagcgcatc

SEQ ID NO: 1632 - DNA, 25 bp, Homo sapiens
gcattacaacaagcgctcgaccatc

SEQ ID NO: 1633 - DNA, 25 bp, Homo sapiens
tcgaccatcacctccagggagatcc

SEQ ID NO: 1634 - DNA, 25 bp, Homo sapiens
ggccaagcacgctgtgtcagagggc

SEQ ID NO: 1635 - DNA, 25 bp, Homo sapiens
gtcagagggcaccaaggccgttacc

SEQ ID NO: 1636 - DNA, 25 bp, Homo sapiens
ttaccaagtacaccagctccaagta

SEQ ID NO: 1637 - DNA, 25 bp, Homo sapiens
gaagggctccaagaaggccgtgacc

SEQ ID NO: 1638 - DNA, 25 bp, Homo sapiens
gttgaccctgcagttcggttatgca

SEQ ID NO: 1639 - DNA, 25 bp, Homo sapiens
gaggattcacttgggtgttgggatc

SEQ ID NO: 1640 - DNA, 25 bp, Homo sapiens
caaatttggattctgtcccaggcct

SEQ ID NO: 1641 - DNA, 25 bp, Homo sapiens
ttctgtcccaggccttactgtaaaa

SEQ ID NO: 1642 - DNA, 25 bp, Homo sapiens
actaggggattgcctttccatatct

SEQ ID NO: 1643 - DNA, 25 bp, Homo sapiens
catatctgctgggggtggagaccct

SEQ ID NO: 1644 - DNA, 25 bp, Homo sapiens
cactcaatcccactggaagcctaat

SEQ ID NO: 1645 - DNA, 25 bp, Homo sapiens
gaagcaatgcctggctggggcagta

SEQ ID NO: 1646 - DNA, 25 bp, Homo sapiens
attccacccaattttgctatgagcc

SEQ ID NO: 1647 - DNA, 25 bp, Homo sapiens
gctatgagcctaaaacctctttaaa

SEQ ID NO: 1648 - DNA, 25 bp, Homo sapiens
acactgtttacaagagcatcaccta

SEQ ID NO: 1649 - DNA, 25 bp, Homo sapiens
tgcaatatgctaatcccactttaca

SEQ ID NO: 1650 - DNA, 25 bp, Homo sapiens
acctgccttttactttcgtgtggat

SEQ ID NO: 1651 - DNA, 25 bp, Homo sapiens
tatgtgaagcattgggtcgggaact

SEQ ID NO: 1652 - DNA, 25 bp, Homo sapiens
gggtcgggaactagctgtagaacac

SEQ ID NO: 1653 - DNA, 25 bp, Homo sapiens
gaataatgtgccagttttttgtagc

SEQ ID NO: 1654 - DNA, 25 bp, Homo sapiens
aaatgctttgtaccagagcacctcc

SEQ ID NO: 1655 - DNA, 25 bp, Homo sapiens
cagagcacctccaaactgcattgag

SEQ ID NO: 1656 - DNA, 25 bp, Homo sapiens
aaagccatgttgactattttacagc

SEQ ID NO: 1657 - DNA, 25 bp, Homo sapiens
acagccactggagttaactaaccct

SEQ ID NO: 1658 - DNA, 25 bp, Homo sapiens
tttcttttgatgtccagttacacca

SEQ ID NO: 1659 - DNA, 25 bp, Homo sapiens
gttacaccatccattctgttaattt

SEQ ID NO: 1660 - DNA, 25 bp, Homo sapiens
aattgtgctcttttctaatccaaag

SEQ ID NO: 1661 - DNA, 25 bp, Homo sapiens
caaagggtatatttgcagcatgctt

SEQ ID NO: 1662 - DNA, 25 bp, Homo sapiens
aataaaaaaaccttcagctgtgcta

SEQ ID NO: 1663 - DNA, 25 bp, Homo sapiens
ctgtgctaaacagtatattacctct

SEQ ID NO: 1664 - DNA, 25 bp, Homo sapiens
atattacctctgtataaaattcttc

SEQ ID NO: 1665 - DNA, 25 bp, Homo sapiens
aattcttcagggagtgtcacctcaa

SEQ ID NO: 1666 - DNA, 25 bp, Homo sapiens
gagtgtcacctcaaatgcaatactt

SEQ ID NO: 1667 - DNA, 25 bp, Homo sapiens
tgcaatactttgggttggtttcttt

SEQ ID NO: 1668 - DNA, 25 bp, Homo sapiens
gtgtgtgagcatgggtacccatttg

SEQ ID NO: 1669 - DNA, 25 bp, Homo sapiens
atgggtacccatttgataagagaaa

SEQ ID NO: 1670 - DNA, 25 bp, Homo sapiens
aattctccattatgttcgtggtgta

SEQ ID NO: 1671 - DNA, 25 bp, Homo sapiens
gttgctgatttagagtcaatctcca

SEQ ID NO: 1672 - DNA, 25 bp, Homo sapiens
tagagtcaatctccaatgttgtgct

SEQ ID NO: 1673 - DNA, 25 bp, Homo sapiens
gggataagtcttatgctatctcagt

SEQ ID NO: 1674 - DNA, 25 bp, Homo sapiens
tatgctatctcagttgacacattga

SEQ ID NO: 1675 - DNA, 25 bp, Homo sapiens
cagttgacacattgaggttattttg

SEQ ID NO: 1676 - DNA, 25 bp, Homo sapiens
gaagctagttggactttgttttgtt

SEQ ID NO: 1677 - DNA, 25 bp, Homo sapiens
tgttttccaaaagttctccactatt

SEQ ID NO: 1678 - DNA, 25 bp, Homo sapiens
aagttctccactattggttttagag

SEQ ID NO: 1679 - DNA, 25 bp, Homo sapiens
agcaaggacatctttcctctgacac

SEQ ID NO: 1680 - DNA, 25 bp, Homo sapiens
acgtgggaatgggtgatatttgtgt

SEQ ID NO: 1681 - DNA, 25 bp, Homo sapiens
gaaatagcctccaatgggaaatatt

SEQ ID NO: 1682 - DNA, 25 bp, Homo sapiens
gtgagaagacatctctttctgctca

SEQ ID NO: 1683 - DNA, 25 bp, Homo sapiens
caggggcagtcgttgagcctttgag

SEQ ID NO: 1684 - DNA, 25 bp, Homo sapiens
ccccaagcaagtctcaaagccagtg

SEQ ID NO: 1685 - DNA, 25 bp, Homo sapiens
cagtgatctctctgactttcaatca

SEQ ID NO: 1686 - DNA, 25 bp, Homo sapiens
accaggggcaagccatgcacatgca

SEQ ID NO: 1687 - DNA, 25 bp, Homo sapiens
tattccttttcaggcctgcagagtg

SEQ ID NO: 1688 - DNA, 25 bp, Homo sapiens
ggctccagaacgaagatccacactt

SEQ ID NO: 1689 - DNA, 25 bp, Homo sapiens
ttgaggactactctcagtcgctgca

SEQ ID NO: 1690 - DNA, 25 bp, Homo sapiens
acgccagaactctgtctggctctcc

SEQ ID NO: 1691 - DNA, 25 bp, Homo sapiens
ccgatcctgttctgagcaagctcga

SEQ ID NO: 1692 - DNA, 25 bp, Homo sapiens
gcaagctcgagtcttcgtggatgat

SEQ ID NO: 1693 - DNA, 25 bp, Homo sapiens
gaacctcatcaattgatagcagtga

SEQ ID NO: 1694 - DNA, 25 bp, Homo sapiens
gtgagtgactgaagcttccaaatca

SEQ ID NO: 1695 - DNA, 25 bp, Homo sapiens
atcaagaaaagccggcaccaagaac

SEQ ID NO: 1696 - DNA, 25 bp, Homo sapiens
ggcaccaagaacttccattctaatc

SEQ ID NO: 1697 - DNA, 25 bp, Homo sapiens
taatctagagctgaccagtttgagc

SEQ ID NO: 1698 - DNA, 25 bp, Homo sapiens
gattgcagtgcagtactggcatttc

SEQ ID NO: 1699 - DNA, 25 bp, Homo sapiens
ttacccttccatttttgtatatcaa

SEQ ID NO: 1700 - DNA, 25 bp, Homo sapiens
gtatatcaaatttccattgtcatta

SEQ ID NO: 1701 - DNA, 25 bp, Homo sapiens
gtatcttgaaactttgtgaactgac

SEQ ID NO: 1702 - DNA, 25 bp, Homo sapiens
gtgaactgacttgctgtatttgcac

SEQ ID NO: 1703 - DNA, 25 bp, Homo sapiens
gtatttgcactttgagctcttgaaa

SEQ ID NO: 1704 - DNA, 25 bp, Homo sapiens
ttctatgcttattgtacttgttatc

SEQ ID NO: 1705 - DNA, 25 bp, Homo sapiens
acacgtttgtatcagagttgctttt

SEQ ID NO: 1706 - DNA, 25 bp, Homo sapiens
gtatcagagttgcttttctaatctt

SEQ ID NO: 1707 - DNA, 25 bp, Homo sapiens
aaattgcttattctaggtctgtaat

SEQ ID NO: 1708 - DNA, 25 bp, Homo sapiens
taatttattaactggctactgggaa

SEQ ID NO: 1709 - DNA, 25 bp, Homo sapiens
attacttattttctggatctatctg

SEQ ID NO: 1710 - DNA, 25 bp, Homo sapiens
aaattatcatactaccggctacatc

SEQ ID NO: 1711 - DNA, 25 bp, Homo sapiens
taccggctacatcaaatcagtcctt

SEQ ID NO: 1712 - DNA, 25 bp, Homo sapiens
tcagtcctttgattccatttggtga

SEQ ID NO: 1713 - DNA, 25 bp, Homo sapiens
attcagtcattgggaaatgccgccc

SEQ ID NO: 1714 - DNA, 25 bp, Homo sapiens
aatgccgcccatttaagtacagtgg

SEQ ID NO: 1715 - DNA, 25 bp, Homo sapiens
ccccttgtgccacacattgcattat

SEQ ID NO: 1716 - DNA, 25 bp, Homo sapiens
cccttgtgccacacattgcattatt

SEQ ID NO: 1717 - DNA, 25 bp, Homo sapiens
cttgtgccacacattgcattattaa

SEQ ID NO: 1718 - DNA, 25 bp, Homo sapiens
gtgccacacattgcattattaaatg

SEQ ID NO: 1719 - DNA, 25 bp, Homo sapiens
gcatccaagcatgatgagccctctc

SEQ ID NO: 1720 - DNA, 25 bp, Homo sapiens
catccaagcatgatgagccctctca

SEQ ID NO: 1721 - DNA, 25 bp, Homo sapiens
agccctctcacggtgcaatggagtg

SEQ ID NO: 1722 - DNA, 25 bp, Homo sapiens
gccctctcacggtgcaatggagtgc

SEQ ID NO: 1723 - DNA, 25 bp, Homo sapiens
ccctctcacggtgcaatggagtgca

SEQ ID NO: 1724 - DNA, 25 bp, Homo sapiens
gcaatggagtgcacggtctgaatct

SEQ ID NO: 1725 - DNA, 25 bp, Homo sapiens
agccaacaggactcttgagctgaag

SEQ ID NO: 1726 - DNA, 25 bp, Homo sapiens
atctatgcaaacacctttcccataa

SEQ ID NO: 1727 - DNA, 25 bp, Homo sapiens
aaccaaaccccatagtacagtgcct

SEQ ID NO: 1728 - DNA, 25 bp, Homo sapiens
tacagtgccttgtcctagtgttcac

SEQ ID NO: 1729 - DNA, 25 bp, Homo sapiens
agtgttcacatgttcagctctgttt

SEQ ID NO: 1730 - DNA, 25 bp, Homo sapiens
gatgccaaggtttccattttcaggg

SEQ ID NO: 1731 - DNA, 25 bp, Homo sapiens
ttaccgctcggttgaatgtgtccac

SEQ ID NO: 1732 - DNA, 25 bp, Homo sapiens
ttggtgacgctgtaaccattccacg

SEQ ID NO: 1733 - DNA, 25 bp, Homo sapiens
cacttggcgcggcctgatactgaaa

SEQ ID NO: 1734 - DNA, 25 bp, Homo sapiens
tagcgtctactcgtgcactgaataa

SEQ ID NO: 1735 - DNA, 25 bp, Homo sapiens
agattttatcactctctgctaagac

SEQ ID NO: 1736 - DNA, 25 bp, Homo sapiens
aagctttatcattgcccatatgtac

SEQ ID NO: 1737 - DNA, 25 bp, Homo sapiens
cgtcaacgacatcttcgagcgcatc

SEQ ID NO: 1738 - DNA, 25 bp, Homo sapiens
cccgcctggcgcattacaacaagcg

SEQ ID NO: 1739 - DNA, 25 bp, Homo sapiens
gcattacaacaagcgctcgaccatc

SEQ ID NO: 1740 - DNA, 25 bp, Homo sapiens
tcgaccatcacctccagggagatcc

SEQ ID NO: 1741 - DNA, 25 bp, Homo sapiens
tcaccaagtacaccagttccaagta

SEQ ID NO: 1742 - DNA, 25 bp, Homo sapiens
gaacttaggaagtctcatctgcctg

SEQ ID NO: 1743 - DNA, 25 bp, Homo sapiens
tgactgtgtggatcccacccaaatc

SEQ ID NO: 1744 - DNA, 25 bp, Homo sapiens
aaatccaactcatcctggtttgctg

SEQ ID NO: 1745 - DNA, 25 bp, Homo sapiens
aggtgtttgcacttcatgttacttt

SEQ ID NO: 1746 - DNA, 25 bp, Homo sapiens
atttacttctgttacagacctagtt

SEQ ID NO: 1747 - DNA, 25 bp, Homo sapiens
tacttgccatggactacctttgcta

SEQ ID NO: 1748 - DNA, 25 bp, Homo sapiens
gaaaaggttgtccaagaagccctgg

SEQ ID NO: 1749 - DNA, 25 bp, Homo sapiens
gaagccctggacaaagccagagaag

SEQ ID NO: 1750 - DNA, 25 bp, Homo sapiens
acaaagccagagaaggccgcacctg

SEQ ID NO: 1751 - DNA, 25 bp, Homo sapiens
cacctgcattgtgattgctcaccgc

SEQ ID NO: 1752 - DNA, 25 bp, Homo sapiens
caccatccagaatgcagacttaata

SEQ ID NO: 1753 - DNA, 25 bp, Homo sapiens
gcagacttaatagtggtgtttcaga

SEQ ID NO: 1754 - DNA, 25 bp, Homo sapiens
agagtcaaggagcatggcacgcatc

SEQ ID NO: 1755 - DNA, 25 bp, Homo sapiens
gtcaaggagcatggcacgcatcagc

SEQ ID NO: 1756 - DNA, 25 bp, Homo sapiens
tcaaggagcatggcacgcatcagca

SEQ ID NO: 1757 - DNA, 25 bp, Homo sapiens
atctatttttcaatggtcagtgtcc

SEQ ID NO: 1758 - DNA, 25 bp, Homo sapiens
tttttcaatggtcagtgtccaggct

SEQ ID NO: 1759 - DNA, 25 bp, Homo sapiens
gccagatttctgctttttggaagaa

SEQ ID NO: 1760 - DNA, 25 bp, Homo sapiens
aagatcctggaatatgtcgaggtta

SEQ ID NO: 1761 - DNA, 25 bp, Homo sapiens
gtcgaggttatattaccaggtattt

SEQ ID NO: 1762 - DNA, 25 bp, Homo sapiens
gaacgtttcaagtatggtggatgcc

SEQ ID NO: 1763 - DNA, 25 bp, Homo sapiens
caagtatggtggatgcctgggcaat

SEQ ID NO: 1764 - DNA, 25 bp, Homo sapiens
gatgcctgggcaatatgaacaattt

SEQ ID NO: 1765 - DNA, 25 bp, Homo sapiens
gagacactggaagaatgcaagaaca

SEQ ID NO: 1766 - DNA, 25 bp, Homo sapiens
gatggtccgaatggtttccaggtgg

SEQ ID NO: 1767 - DNA, 25 bp, Homo sapiens
aattatggaacccagctcaatgctg

SEQ ID NO: 1768 - DNA, 25 bp, Homo sapiens
aatgctgtgaataactccctgactc

SEQ ID NO: 1769 - DNA, 25 bp, Homo sapiens
cctgactccgcaatcaaccaaggtt

SEQ ID NO: 1770 - DNA, 25 bp, Homo sapiens
gattggatagcatttcatgcctatg

SEQ ID NO: 1771 - DNA, 25 bp, Homo sapiens
catgcctatgttaatatttgtgctt

SEQ ID NO: 1772 - DNA, 25 bp, Homo sapiens
ttatatgtatacgtgatgcctttgt

SEQ ID NO: 1773 - DNA, 25 bp, Homo sapiens
gtgatgcctttgtagcatactgcta

SEQ ID NO: 1774 - DNA, 25 bp, Homo sapiens
aaatgatggttggaagaatgcggct

SEQ ID NO: 1775 - DNA, 25 bp, Homo sapiens
gaatgcggctcatatttaccaagtc

SEQ ID NO: 1776 - DNA, 25 bp, Homo sapiens
gcggctcatatttaccaagtctttc

SEQ ID NO: 1777 - DNA, 25 bp, Homo sapiens
ttaccaagtctttctgaacgccttc

SEQ ID NO: 1778 - DNA, 25 bp, Homo sapiens
gccttctgcattcatgcatccatgt

SEQ ID NO: 1779 - DNA, 25 bp, Homo sapiens
tcatgcatccatgttctttctagga

SEQ ID NO: 1780 - DNA, 25 bp, Homo sapiens
catgttctttctaggattggatagc

SEQ ID NO: 1781 - DNA, 25 bp, Homo sapiens
tcagaaacctaaacacccaacaaca

SEQ ID NO: 1782 - DNA, 25 bp, Homo sapiens
acaggaattattgccatcacattgg

SEQ ID NO: 1783 - DNA, 25 bp, Homo sapiens
atgtcacgaagttcacctagctggt

SEQ ID NO: 1784 - DNA, 25 bp, Homo sapiens
ttaaatacaacttttctgacctcaa

SEQ ID NO: 1785 - DNA, 25 bp, Homo sapiens
tgacctcaagagtcctttgcactac

SEQ ID NO: 1786 - DNA, 25 bp, Homo sapiens
gcagagcagctctttttgaaggaca

SEQ ID NO: 1787 - DNA, 25 bp, Homo sapiens
aaaacctcgtaatcaacttgactca

SEQ ID NO: 1788 - DNA, 25 bp, Homo sapiens
gactcaagattgactctacagactc

SEQ ID NO: 1789 - DNA, 25 bp, Homo sapiens
atatgttggatgcactcgtcaaata

SEQ ID NO: 1790 - DNA, 25 bp, Homo sapiens
gattcataaccaccagcttaatttc

SEQ ID NO: 1791 - DNA, 25 bp, Homo sapiens
gaaaccagccttaaacctgatttat

SEQ ID NO: 1792 - DNA, 25 bp, Homo sapiens
caaagttgaaagtgtcctttctctc

SEQ ID NO: 1793 - DNA, 25 bp, Homo sapiens
ctccccgtcgtaaacgctgaggaat

SEQ ID NO: 1794 - DNA, 25 bp, Homo sapiens
ggcaagaatgccatgatgttcttta

SEQ ID NO: 1795 - DNA, 25 bp, Homo sapiens
gagttttaagggcttgtctcattat

SEQ ID NO: 1796 - DNA, 25 bp, Homo sapiens
gggcttgtctcattatagaggcaca

SEQ ID NO: 1797 - DNA, 25 bp, Homo sapiens
ggcacattgtggctgtgtaggtgaa

SEQ ID NO: 1798 - DNA, 25 bp, Homo sapiens
ataggtgtactttttccaatgctgc

SEQ ID NO: 1799 - DNA, 25 bp, Homo sapiens
tccaatgctgctccaagttacttaa

SEQ ID NO: 1800 - DNA, 25 bp, Homo sapiens
ataaacatgccattctctttcagct

SEQ ID NO: 1801 - DNA, 25 bp, Homo sapiens
tctttcagctgtaatgttcttaaaa

SEQ ID NO: 1802 - DNA, 25 bp, Homo sapiens
ttattcttgaatgtactgtgatgtc

SEQ ID NO: 1803 - DNA, 25 bp, Homo sapiens
agtatgccttcttaccagcaatagt

SEQ ID NO: 1804 - DNA, 25 bp, Homo sapiens
atcatgccagatttttgccaagatc

SEQ ID NO: 1805 - DNA, 25 bp, Homo sapiens
ccaagatcagtgtttcctcaacatg

SEQ ID NO: 1806 - DNA, 25 bp, Homo sapiens
gtatagtgtgctcttgtacctctac

SEQ ID NO: 1807 - DNA, 25 bp, Homo sapiens
gtgctcttgtacctctacatagatt

SEQ ID NO: 1808 - DNA, 25 bp, Homo sapiens
agcagttacacatttatctaaagga

SEQ ID NO: 1809 - DNA, 25 bp, Homo sapiens
aatgcatgtttaccaaaatggctgt

SEQ ID NO: 1810 - DNA, 25 bp, Homo sapiens
ttagacatcgatcacatctggagac

SEQ ID NO: 1811 - DNA, 25 bp, Homo sapiens
gtaggcgagctaacacagtgtacct

SEQ ID NO: 1812 - DNA, 25 bp, Homo sapiens
acacagtgtacctaattgcagaatt

SEQ ID NO: 1813 - DNA, 25 bp, Homo sapiens
agttgtataacattttcatatctta

SEQ ID NO: 1814 - DNA, 25 bp, Homo sapiens
tattttggtacctgtgcttgccaca

SEQ ID NO: 1815 - DNA, 25 bp, Homo sapiens
ttgatagatttctctttgacttcca

SEQ ID NO: 1816 - DNA, 25 bp, Homo sapiens
ttgacttccaagacctagcagttat

SEQ ID NO: 1817 - DNA, 25 bp, Homo sapiens
gtcctagtgcttccgaatcatttaa

SEQ ID NO: 1818 - DNA, 25 bp, Homo sapiens
aatggcattgtcggatatcttttac

SEQ ID NO: 1819 - DNA, 25 bp, Homo sapiens
atcttttacatttcaattgcaatcc

SEQ ID NO: 1820 - DNA, 25 bp, Homo sapiens
agtacttaactgtagtcttctccat

SEQ ID NO: 1821 - DNA, 25 bp, Homo sapiens
gtagtcttctccatgaattacacgt

SEQ ID NO: 1822 - DNA, 25 bp, Homo sapiens
gcctctagcttatagtttcatccct

SEQ ID NO: 1823 - DNA, 25 bp, Homo sapiens
gcctgcgtgagtctgtacagggata

SEQ ID NO: 1824 - DNA, 25 bp, Homo sapiens
ggtccaaactactctttgcactact

SEQ ID NO: 1825 - DNA, 25 bp, Homo sapiens
gatgcaattggttctcctgcattga

SEQ ID NO: 1826 - DNA, 25 bp, Homo sapiens
gttaacatttatacttgccttggac

SEQ ID NO: 1827 - DNA, 25 bp, Homo sapiens
tacttgccttggactgtagaacaga

SEQ ID NO: 1828 - DNA, 25 bp, Homo sapiens
tacaatcaagtcattttacctttac

SEQ ID NO: 1829 - DNA, 25 bp, Homo sapiens
atagcatgatgctctgcagttttat

SEQ ID NO: 1830 - DNA, 25 bp, Homo sapiens
taaccatacaactctcatttcctta

SEQ ID NO: 1831 - DNA, 25 bp, Homo sapiens
aactctcatttccttagtaagccaa

SEQ ID NO: 1832 - DNA, 25 bp, Homo sapiens
aatgtttaacattttgtgccaattt

SEQ ID NO: 1833 - DNA, 25 bp, Homo sapiens
gccaatttgttcctgtattcatgta

SEQ ID NO: 1834 - DNA, 25 bp, Homo sapiens
gttacagatctgactcttcattttt

SEQ ID NO: 1835 - DNA, 25 bp, Homo sapiens
agttccttgttacatcatggtcatt

SEQ ID NO: 1836 - DNA, 25 bp, Homo sapiens
aatttccacagatacttcccttaga

SEQ ID NO: 1837 - DNA, 25 bp, Homo sapiens
tgagcgaggccttgtcaattttaag

SEQ ID NO: 1838 - DNA, 25 bp, Homo sapiens
taggaaggaccacaacatgacccgt

SEQ ID NO: 1839 - DNA, 25 bp, Homo sapiens
tacacactttatttacttcgttttg

SEQ ID NO: 1840 - DNA, 25 bp, Homo sapiens
gttggcttctgtttctagttgagga

SEQ ID NO: 1841 - DNA, 25 bp, Homo sapiens
tcctctttttccatcataattctaa

SEQ ID NO: 1842 - DNA, 25 bp, Homo sapiens
gatttgcccatttacacttttgaga

SEQ ID NO: 1843 - DNA, 25 bp, Homo sapiens
gtaaataaccccattctttgcttga

SEQ ID NO: 1844 - DNA, 25 bp, Homo sapiens
gtattttcccaatagcactttcatt

SEQ ID NO: 1845 - DNA, 25 bp, Homo sapiens
attgccagtgtctttctttggtgcc

SEQ ID NO: 1846 - DNA, 25 bp, Homo sapiens
ttcagcattcttagcctgtggcaat

SEQ ID NO: 1847 - DNA, 25 bp, Homo sapiens
aacaacgacaaaaagctccaagctg

SEQ ID NO: 1848 - DNA, 25 bp, Homo sapiens
aaagctccaagctgcagtggattta

SEQ ID NO: 1849 - DNA, 25 bp, Homo sapiens
ggctaaaactacctcatactttcct

SEQ ID NO: 1850 - DNA, 25 bp, Homo sapiens
actacctcatactttccttggaaga

SEQ ID NO: 1851 - DNA, 25 bp, Homo sapiens
aaagcaaatgatttccatattcctg

SEQ ID NO: 1852 - DNA, 25 bp, Homo sapiens
atttccatattcctgattgatcttt

SEQ ID NO: 1853 - DNA, 25 bp, Homo sapiens
acaagtttcttgttcatattgtgaa

SEQ ID NO: 1854 - DNA, 25 bp, Homo sapiens
gatttgttaaactggtccttagtca

SEQ ID NO: 1855 - DNA, 25 bp, Homo sapiens
aactggtccttagtcatttgtataa

SEQ ID NO: 1856 - DNA, 25 bp, Homo sapiens
atttgtatagccttctagaatcaga

SEQ ID NO: 1857 - DNA, 25 bp, Homo sapiens
gaaataacctttttgcatattcttt

SEQ ID NO: 1858 - DNA, 25 bp, Homo sapiens
taagctagttttctgaggtgttttc

SEQ ID NO: 1859 - DNA, 25 bp, Homo sapiens
gtgttttcacacgtctttttatagt

SEQ ID NO: 1860 - DNA, 25 bp, Homo sapiens
ttatagttacttcatcttagatttt

SEQ ID NO: 1861 - DNA, 25 bp, Homo sapiens
aagggatatgacttcctactaagga

SEQ ID NO: 1862 - DNA, 25 bp, Homo sapiens
gtttaccacaacaattctgactaca

SEQ ID NO: 1863 - DNA, 25 bp, Homo sapiens
ttgaggaggatatttggctactgta

SEQ ID NO: 1864 - DNA, 25 bp, Homo sapiens
ggctactgtaaacatggctggtgga

SEQ ID NO: 1865 - DNA, 25 bp, Homo sapiens
ggcaagccgaaaccacttggctctg

SEQ ID NO: 1866 - DNA, 25 bp, Homo sapiens
ggctctggaaatctaagttcatact

SEQ ID NO: 1867 - DNA, 25 bp, Homo sapiens
tggtttaattaagctctctcctgac

SEQ ID NO: 1868 - DNA, 25 bp, Homo sapiens
tgacaacccccagaattaaatgaac

SEQ ID NO: 1869 - DNA, 25 bp, Homo sapiens
ctcgagggttcatgcagtcagtgtt

SEQ ID NO: 1870 - DNA, 25 bp, Homo sapiens
gtcagtgttataccaaacccagtgt

SEQ ID NO: 1871 - DNA, 25 bp, Homo sapiens
aaaaatgcgcatctctttctttgtt

SEQ ID NO: 1872 - DNA, 25 bp, Homo sapiens
ttcaggacctcatcattatgtgggg

SEQ ID NO: 1873 - DNA, 25 bp, Homo sapiens
caggtaagagatggccttcttggct

SEQ ID NO: 1874 - DNA, 25 bp, Homo sapiens
ggctgccacaatcagaaatcacgca

SEQ ID NO: 1875 - DNA, 25 bp, Homo sapiens
gcattttgggtaggcggcctccagt

SEQ ID NO: 1876 - DNA, 25 bp, Homo sapiens
ccagttttcctttgagtcgcgaacg

SEQ ID NO: 1877 - DNA, 25 bp, Homo sapiens
gtcgcgaacgctgtgcgtttgtcag

SEQ ID NO: 1878 - DNA, 25 bp, Homo sapiens
actacgagttgatctcggccagcca

SEQ ID NO: 1879 - DNA, 25 bp, Homo sapiens
tcggccagccaaagacacacgacaa

SEQ ID NO: 1880 - DNA, 25 bp, Homo sapiens
gttctactccatacagttcacactg

SEQ ID NO: 1881 - DNA, 25 bp, Homo sapiens
gattgtgacacattcttagtagcta

SEQ ID NO: 1882 - DNA, 25 bp, Homo sapiens
gctagtgtctgttctagtcactgca

SEQ ID NO: 1883 - DNA, 25 bp, Homo sapiens
agtcactgcactggagtctacgagc

SEQ ID NO: 1884 - DNA, 25 bp, Homo sapiens
gagtctacgagccggaactcgctat

SEQ ID NO: 1885 - DNA, 25 bp, Homo sapiens
cggaactcgctatatgcacgtgtgt

SEQ ID NO: 1886 - DNA, 25 bp, Homo sapiens
acgtgtgtgtgtccgtatgtaagaa

SEQ ID NO: 1887 - DNA, 25 bp, Homo sapiens
gaaagtgtgcaccgagtgactgaat

SEQ ID NO: 1888 - DNA, 25 bp, Homo sapiens
gactgatatcgagcattctgcccac

SEQ ID NO: 1889 - DNA, 25 bp, Homo sapiens
gctttaacaacccattgagcagtca

SEQ ID NO: 1890 - DNA, 25 bp, Homo sapiens
gggaatgtgagtaagcttgctgcca

SEQ ID NO: 1891 - DNA, 25 bp, Homo sapiens
actgtccttttgggcttctataaat

SEQ ID NO: 1892 - DNA, 25 bp, Homo sapiens
atatgtaatcgtgccagtctgttct

SEQ ID NO: 1893 - DNA, 25 bp, Homo sapiens
cagtctgttctctgcatgacataat

SEQ ID NO: 1894 - DNA, 25 bp, Homo sapiens
atgacataattttccagcaatagct

SEQ ID NO: 1895 - DNA, 25 bp, Homo sapiens
gctgtgtggtttttgtaatcctatc

SEQ ID NO: 1896 - DNA, 25 bp, Homo sapiens
gtaatcctatcatctagtcagttca

SEQ ID NO: 1897 - DNA, 25 bp, Homo sapiens
gtcagttcaagatcttgcaacactg

SEQ ID NO: 1898 - DNA, 25 bp, Homo sapiens
caacactgtgtgattctttgctccg

SEQ ID NO: 1899 - DNA, 25 bp, Homo sapiens
ttgctccgtagttcagtcttgttga

SEQ ID NO: 1900 - DNA, 25 bp, Homo sapiens
gacacaggtgtttactttcctgttc

SEQ ID NO: 1901 - DNA, 25 bp, Homo sapiens
tttcctgttcttgcatctagtttca

SEQ ID NO: 1902 - DNA, 25 bp, Homo sapiens
gaatccagatggcatggttgctcta

SEQ ID NO: 1903 - DNA, 25 bp, Homo sapiens
ggttgctctattggactaccgtgag

SEQ ID NO: 1904 - DNA, 25 bp, Homo sapiens
ctaccgtgaggatggtgtgacccca

SEQ ID NO: 1905 - DNA, 25 bp, Homo sapiens
gtgaccccatatatgattttcttta

SEQ ID NO: 1906 - DNA, 25 bp, Homo sapiens
atgtggcaattattttggatctatc

SEQ ID NO: 1907 - DNA, 25 bp, Homo sapiens
gactgatgtcatcttgagctcttca

SEQ ID NO: 1908 - DNA, 25 bp, Homo sapiens
ttcccttgtactgtagtttgttttg

SEQ ID NO: 1909 - DNA, 25 bp, Homo sapiens
gagctcttcatttattttgactgtg

SEQ ID NO: 1910 - DNA, 25 bp, Homo sapiens
tttggagtggaggcattgtttttaa

SEQ ID NO: 1911 - DNA, 25 bp, Homo sapiens
gtttgttttgaatggcatgtatttg

SEQ ID NO: 1912 - DNA, 25 bp, Homo sapiens
taattctaggtattttgtttgcttc

SEQ ID NO: 1913 - DNA, 25 bp, Homo sapiens
gatacaacgtgtttcctaaaagtag

SEQ ID NO: 1914 - DNA, 25 bp, Homo sapiens
cttgacttaactgcttccctgaagt

SEQ ID NO: 1915 - DNA, 25 bp, Homo sapiens
gacttaactgcttccctgaagtacc

SEQ ID NO: 1916 - DNA, 25 bp, Homo sapiens
taactgcttccctgaagtaccgtga

SEQ ID NO: 1917 - DNA, 25 bp, Homo sapiens
gcttccctgaagtaccgtgaggttc

SEQ ID NO: 1918 - DNA, 25 bp, Homo sapiens
agtaccgtgaggttcctgatgtgcg

SEQ ID NO: 1919 - DNA, 25 bp, Homo sapiens
ccgtgaggttcctgatgtgcgggcg

SEQ ID NO: 1920 - DNA, 25 bp, Homo sapiens
ttcctgatgtgcgggcggtagacgg

SEQ ID NO: 1921 - DNA, 25 bp, Homo sapiens
atgtgcgggcggtagacggtaggct

SEQ ID NO: 1922 - DNA, 25 bp, Homo sapiens
cgggcggtagacggtaggcttatgc

SEQ ID NO: 1923 - DNA, 25 bp, Homo sapiens
tagacggtaggcttatgcggcacgc

SEQ ID NO: 1924 - DNA, 25 bp, Homo sapiens
tattcatacgtaaaattttggatta

SEQ ID NO: 1925 - DNA, 25 bp, Homo sapiens
gaaccacctcaatgcaaagattcta

SEQ ID NO: 1926 - DNA, 25 bp, Homo sapiens
gagggtaacaagcgaataacatgta

SEQ ID NO: 1927 - DNA, 25 bp, Homo sapiens
ccaccaaaatgcttacatccgtgtg

SEQ ID NO: 1928 - DNA, 25 bp, Homo sapiens
acatccgtgtgtaatatcccgagaa

SEQ ID NO: 1929 - DNA, 25 bp, Homo sapiens
tccgtgtgtaatatcccgagaaatt

SEQ ID NO: 1930 - DNA, 25 bp, Homo sapiens
aacatagcattaaggtggacagcca

SEQ ID NO: 1931 - DNA, 25 bp, Homo sapiens
gcattaaggtggacagccaaacaga

SEQ ID NO: 1932 - DNA, 25 bp, Homo sapiens
gaatttgtgtgtaaacggggatatc

SEQ ID NO: 1933 - DNA, 25 bp, Homo sapiens
tcacgttctcacacattgcgaacaa

SEQ ID NO: 1934 - DNA, 25 bp, Homo sapiens
cacacattgcgaacaacatgttggg

SEQ ID NO: 1935 - DNA, 25 bp, Homo sapiens
aagcgcaagcgcagtcgtaaggaga

SEQ ID NO: 1936 - DNA, 25 bp, Homo sapiens
gcgcagtcgtaaggagagctactcc

SEQ ID NO: 1937 - DNA, 25 bp, Homo sapiens
gtgctaaaacaggttcaccccgata

SEQ ID NO: 1938 - DNA, 25 bp, Homo sapiens
taaaacaggttcaccccgatactgg

SEQ ID NO: 1939 - DNA, 25 bp, Homo sapiens
aagcacgcagtgtccgaaggtacca

SEQ ID NO: 1940 - DNA, 25 bp, Homo sapiens
gtccgaaggtaccaaggctgtcacc

SEQ ID NO: 1941 - DNA, 25 bp, Homo sapiens
ggctgtcaccaagtatacaagctcc

SEQ ID NO: 1942 - DNA, 25 bp, Homo sapiens
tacaagctccaagtaaatgtgtgct

SEQ ID NO: 1943 - DNA, 25 bp, Homo sapiens
tcagctcctgctccgaagaagggtt

SEQ ID NO: 1944 - DNA, 25 bp, Homo sapiens
cctgctccgaagaagggttccaaga

SEQ ID NO: 1945 - DNA, 25 bp, Homo sapiens
gttccaagaaggctgtgaccaaggc

SEQ ID NO: 1946 - DNA, 25 bp, Homo sapiens
gtgatgcgttggaaggttaatcgaa

SEQ ID NO: 1947 - DNA, 25 bp, Homo sapiens
ccatccttacccctatttaatgtag

SEQ ID NO: 1948 - DNA, 25 bp, Homo sapiens
aacaataccatatagcttgcttttt

SEQ ID NO: 1949 - DNA, 25 bp, Homo sapiens
ctttgtccatatttctacttataac

SEQ ID NO: 1950 - DNA, 25 bp, Homo sapiens
tatttctacttataacctgttgcta

SEQ ID NO: 1951 - DNA, 25 bp, Homo sapiens
tgtatctcttgttatctgcatctca

SEQ ID NO: 1952 - DNA, 25 bp, Homo sapiens
gttatctgcatctcattgtttattg

SEQ ID NO: 1953 - DNA, 25 bp, Homo sapiens
gaaccaatctacaagtctctgtctt

SEQ ID NO: 1954 - DNA, 25 bp, Homo sapiens
agcctctcggtggtgggattatgaa

SEQ ID NO: 1955 - DNA, 25 bp, Homo sapiens
ttatgaatgatttttctccttttgc

SEQ ID NO: 1956 - DNA, 25 bp, Homo sapiens
ttctccttttgcttgttagtatttt

SEQ ID NO: 1957 - DNA, 25 bp, Homo sapiens
ccttcagttcccggtagggcgagtg

SEQ ID NO: 1958 - DNA, 25 bp, Homo sapiens
gcatcgcttgctgcgcaaaggcaac

SEQ ID NO: 1959 - DNA, 25 bp, Homo sapiens
tcctcgagtatctgaccgccgagat

SEQ ID NO: 1960 - DNA, 25 bp, Homo sapiens
cgccgagatcctggagctggcgggc

SEQ ID NO: 1961 - DNA, 25 bp, Homo sapiens
caggcaggagtttctctcggtgact

SEQ ID NO: 1962 - DNA, 25 bp, Homo sapiens
aagctgctgggcaaagtcaccatcg

SEQ ID NO: 1963 - DNA, 25 bp, Homo sapiens
tcttgcctaacatccaggccgtact

SEQ ID NO: 1964 - DNA, 25 bp, Homo sapiens
aaagggcaagtgaggctgacgtccg

SEQ ID NO: 1965 - DNA, 25 bp, Homo sapiens
gcgtctcgaaggggcacctgtgaac

SEQ ID NO: 1966 - DNA, 25 bp, Homo sapiens
tactatcgctgtcatgtctggtcgt

SEQ ID NO: 1967 - DNA, 25 bp, Homo sapiens
gcaagcaaggaggcaaggcccgcgc

SEQ ID NO: 1968 - DNA, 25 bp, Homo sapiens
ccctccctttggatgctggtgaata

SEQ ID NO: 1969 - DNA, 25 bp, Homo sapiens
ttggatgctggtgaatactgtgtgc

SEQ ID NO: 1970 - DNA, 25 bp, Homo sapiens
gatgggatatgatgcataggcttgg

SEQ ID NO: 1971 - DNA, 25 bp, Homo sapiens
tgatggtttccctaaagttattacg

SEQ ID NO: 1972 - DNA, 25 bp, Homo sapiens
gacccctgctttcgaatttacatgt

SEQ ID NO: 1973 - DNA, 25 bp, Homo sapiens
atgttcatgatgtgcccttgttgta

SEQ ID NO: 1974 - DNA, 25 bp, Homo sapiens
atgatgtgcccttgttgtaaacctt

SEQ ID NO: 1975 - DNA, 25 bp, Homo sapiens
tgtaaacctttacctgtcacttgtt

SEQ ID NO: 1976 - DNA, 25 bp, Homo sapiens
ctgtcacttgtttacgtgggtctcc

SEQ ID NO: 1977 - DNA, 25 bp, Homo sapiens
cacttgtttacgtgggtctcctatt

SEQ ID NO: 1978 - DNA, 25 bp, Homo sapiens
attgtgtttttgaaccagtctgtaa

SEQ ID NO: 1979 - DNA, 25 bp, Homo sapiens
taatcatttctgggttcactgcgac

SEQ ID NO: 1980 - DNA, 25 bp, Homo sapiens
cactgcgactcactgtagtgctggg

SEQ ID NO: 1981 - DNA, 25 bp, Homo sapiens
atcccccttgtaacactggaactga

SEQ ID NO: 1982 - DNA, 25 bp, Homo sapiens
gaggagaaatgccacatacctttcc

SEQ ID NO: 1983 - DNA, 25 bp, Homo sapiens
atacctttcccatgggacctgtggt

SEQ ID NO: 1984 - DNA, 25 bp, Homo sapiens
cgagcagacttttgttctcggcgct

SEQ ID NO: 1985 - DNA, 25 bp, Homo sapiens
ggcgctcctcacgatggagtttcat

SEQ ID NO: 1986 - DNA, 25 bp, Homo sapiens
gtttcatgcttcattttcacatctc

SEQ ID NO: 1987 - DNA, 25 bp, Homo sapiens
gagtacgtgccttaatctttatctt

SEQ ID NO: 1988 - DNA, 25 bp, Homo sapiens
atgaacagagtgcctcctggtacac

SEQ ID NO: 1989 - DNA, 25 bp, Homo sapiens
agaatgggatttactctgctttacc

SEQ ID NO: 1990 - DNA, 25 bp, Homo sapiens
caccaagacgactgcttcagcttct

SEQ ID NO: 1991 - DNA, 25 bp, Homo sapiens
tctcttatccttactttctttaata

SEQ ID NO: 1992 - DNA, 25 bp, Homo sapiens
aaaggtgccacaatgcccagtattg

SEQ ID NO: 1993 - DNA, 25 bp, Homo sapiens
agctttcattcattctggagtctac

SEQ ID NO: 1994 - DNA, 25 bp, Homo sapiens
attctgtgaaatgcctctccacgtt

SEQ ID NO: 1995 - DNA, 25 bp, Homo sapiens
tctccacgttgcatatgtcacactt

SEQ ID NO: 1996 - DNA, 25 bp, Homo sapiens
gtctgcacataactcttttttcaca

SEQ ID NO: 1997 - DNA, 25 bp, Homo sapiens
gccacaacagcacagtcagcgggtg

SEQ ID NO: 1998 - DNA, 25 bp, Homo sapiens
gtcagcgggtgaattacaggtgcct

SEQ ID NO: 1999 - DNA, 25 bp, Homo sapiens
gtaatctgatcttgtctgtatcgcc

SEQ ID NO: 2000 - DNA, 25 bp, Homo sapiens
agaattgcaggccactcatgtcagt

SEQ ID NO: 2001 - DNA, 25 bp, Homo sapiens
tttccatagcaggtattttctacta

SEQ ID NO: 2002 - DNA, 25 bp, Homo sapiens
tctgaagtctttttcatgcccttgt

SEQ ID NO: 2003 - DNA, 25 bp, Homo sapiens
agcttgacttatttttttctctctc

SEQ ID NO: 2004 - DNA, 25 bp, Homo sapiens
ggagaaattttctcagcattttgca

SEQ ID NO: 2005 - DNA, 25 bp, Homo sapiens
gcattttgcatgttctttctaatct

SEQ ID NO: 2006 - DNA, 25 bp, Homo sapiens
gttctttctaatctttgttggtctg

SEQ ID NO: 2007 - DNA, 25 bp, Homo sapiens
tcaaaaatttttccactatgtcttt

SEQ ID NO: 2008 - DNA, 25 bp, Homo sapiens
tatgtcttttttctagtggctactg

SEQ ID NO: 2009 - DNA, 25 bp, Homo sapiens
gtggctactgttttagttttctagt

SEQ ID NO: 2010 - DNA, 25 bp, Homo sapiens
atctctgacaagctttcgtatggtt

SEQ ID NO: 2011 - DNA, 25 bp, Homo sapiens
ggttttgttatatcttcatctacat

SEQ ID NO: 2012 - DNA, 25 bp, Homo sapiens
tatattcatcttttcagggtaaatt

SEQ ID NO: 2013 - DNA, 25 bp, Homo sapiens
gagtttctcgtaatgctcattttta

SEQ ID NO: 2014 - DNA, 25 bp, Homo sapiens
ctcatttttacatgctgctactagc

SEQ ID NO: 2015 - DNA, 25 bp, Homo sapiens
gtgccattgcaatcgtaagtagact

SEQ ID NO: 2016 - DNA, 25 bp, Homo sapiens
gtagactatgtatttcctataatga

SEQ ID NO: 2017 - DNA, 25 bp, Homo sapiens
tttaacttgcctagatccctgtatt

SEQ ID NO: 2018 - DNA, 25 bp, Homo sapiens
tagatccctgtattccaaaacctgc

SEQ ID NO: 2019 - DNA, 25 bp, Homo sapiens
tgtattccaaaacctgctgcatcat

SEQ ID NO: 2020 - DNA, 25 bp, Homo sapiens
catgatttctatgtttcttaatgat

SEQ ID NO: 2021 - DNA, 25 bp, Homo sapiens
ggaatttgtgcgttcatgctttttc

SEQ ID NO: 2022 - DNA, 25 bp, Homo sapiens
gttcatgctttttcgtattctttat

SEQ ID NO: 2023 - DNA, 25 bp, Homo sapiens
catgctgacatgttctgccacaggc

SEQ ID NO: 2024 - DNA, 25 bp, Homo sapiens
gcgtcatctacaagctgggtggcga

SEQ ID NO: 2025 - DNA, 25 bp, Homo sapiens
actggagcactgccatggactgtgg

SEQ ID NO: 2026 - DNA, 25 bp, Homo sapiens
aagggccacccgaggaagcagtatt

SEQ ID NO: 2027 - DNA, 25 bp, Homo sapiens
aggacaggaaaaccacgtgctccac

SEQ ID NO: 2028 - DNA, 25 bp, Homo sapiens
tggcaaggaggctcaggtgcttcca

SEQ ID NO: 2029 - DNA, 25 bp, Homo sapiens
gtgcttccatctgtggtgactggaa

SEQ ID NO: 2030 - DNA, 25 bp, Homo sapiens
ggaatgggacccacgtggagtaggt

SEQ ID NO: 2031 - DNA, 25 bp, Homo sapiens
gagtaggtgacatatgcttcccaga

SEQ ID NO: 2032 - DNA, 25 bp, Homo sapiens
gtggctgtgccaggagtacatgtga

SEQ ID NO: 2033 - DNA, 25 bp, Homo sapiens
atatatgtgcccatttatctttttc

SEQ ID NO: 2034 - DNA, 25 bp, Homo sapiens
gacaacaatgaagtagcccctgaac

SEQ ID NO: 2035 - DNA, 25 bp, Homo sapiens
gtagcccctgaacagcatggagttg

SEQ ID NO: 2036 - DNA, 25 bp, Homo sapiens
gagttgctgtgagtttgttcgttgc

SEQ ID NO: 2037 - DNA, 25 bp, Homo sapiens
gttcgttgcagacctttgtgttggg

SEQ ID NO: 2038 - DNA, 25 bp, Homo sapiens
ggtcctgggaatctgagctttgttc

SEQ ID NO: 2039 - DNA, 25 bp, Homo sapiens
ctttgttccctgtgcatggtggata

SEQ ID NO: 2040 - DNA, 25 bp, Homo sapiens
gggatagaccttgtgacagaccaat

SEQ ID NO: 2041 - DNA, 25 bp, Homo sapiens
gacagaccaattctgtgacccctgt

SEQ ID NO: 2042 - DNA, 25 bp, Homo sapiens
tgacccctgtcttctgggtcacatt

SEQ ID NO: 2043 - DNA, 25 bp, Homo sapiens
aaatgtgtatgtgtccttgtaaatg

SEQ ID NO: 2044 - DNA, 25 bp, Homo sapiens
gcaagaatgccacgtactcagagta

SEQ ID NO: 2045 - DNA, 25 bp, Homo sapiens
tgtcctgcacactgtaggatgctta

SEQ ID NO: 2046 - DNA, 25 bp, Homo sapiens
gatgcttaaaggtatccctggcctc

SEQ ID NO: 2047 - DNA, 25 bp, Homo sapiens
cccagtcagacatgacctcagagtc

SEQ ID NO: 2048 - DNA, 25 bp, Homo sapiens
ctcagagtctctgtgtctcctagaa

SEQ ID NO: 2049 - DNA, 25 bp, Homo sapiens
ctcctagaagcctgacagagacccc

SEQ ID NO: 2050 - DNA, 25 bp, Homo sapiens
tgggtgggtggcgggctagagaccc

SEQ ID NO: 2051 - DNA, 25 bp, Homo sapiens
ccctccgcactaacagtgttctcag

SEQ ID NO: 2052 - DNA, 25 bp, Homo sapiens
gcctggtgattctgctctccaggga

SEQ ID NO: 2053 - DNA, 25 bp, Homo sapiens
ctcccttttcgttgcctgaggagct

SEQ ID NO: 2054 - DNA, 25 bp, Homo sapiens
ggagctggtggtttcatgagttaat

SEQ ID NO: 2055 - DNA, 25 bp, Homo sapiens
gtggaaaagcacgccaaagccttat

SEQ ID NO: 2056 - DNA, 25 bp, Homo sapiens
atgctgtgaatgcagcttgcttctc

SEQ ID NO: 2057 - DNA, 25 bp, Homo sapiens
gtccagcttcaaaagttacttgcca

SEQ ID NO: 2058 - DNA, 25 bp, Homo sapiens
agattttgcacttctgaattcaggt

SEQ ID NO: 2059 - DNA, 25 bp, Homo sapiens
tctgcttagaggactgtgacttgaa

SEQ ID NO: 2060 - DNA, 25 bp, Homo sapiens
tgagctttttggtagcgtccacaat

SEQ ID NO: 2061 - DNA, 25 bp, Homo sapiens
ataggcgagatccgtgttctccatt

SEQ ID NO: 2062 - DNA, 25 bp, Homo sapiens
tgtagaccaatttaactgctgtgtt

SEQ ID NO: 2063 - DNA, 25 bp, Homo sapiens
ttagtgcttaatctttgcctcatgt

SEQ ID NO: 2064 - DNA, 25 bp, Homo sapiens
ttctctcaattctgtagactctcgc

SEQ ID NO: 2065 - DNA, 25 bp, Homo sapiens
gcactgaagatctttgctggacctt

SEQ ID NO: 2066 - DNA, 25 bp, Homo sapiens
ctggaccttcttctcttcagaagat

SEQ ID NO: 2067 - DNA, 25 bp, Homo sapiens
cacctgtgctctgattaaatctaca

SEQ ID NO: 2068 - DNA, 25 bp, Homo sapiens
agtaatccattacacttttctatgt

SEQ ID NO: 2069 - DNA, 25 bp, Homo sapiens
attctggctttagatcccgacattc

SEQ ID NO: 2070 - DNA, 25 bp, Homo sapiens
ccgacattcactcctgtgcaaatta

SEQ ID NO: 2071 - DNA, 25 bp, Homo sapiens
gtacattcactccctcaagagaatc

SEQ ID NO: 2072 - DNA, 25 bp, Homo sapiens
atttcaatcaatcattccatctaaa

SEQ ID NO: 2073 - DNA, 25 bp, Homo sapiens
aaatctctacaggactacataacat

SEQ ID NO: 2074 - DNA, 25 bp, Homo sapiens
aaacgattgcctatctgaattttta

SEQ ID NO: 2075 - DNA, 25 bp, Homo sapiens
tgaatttttatacctaccactactt

SEQ ID NO: 2076 - DNA, 25 bp, Homo sapiens
ggaaactatatccatatcgcttttg

SEQ ID NO: 2077 - DNA, 25 bp, Homo sapiens
atcgcttttggtgtcagattgtatc

SEQ ID NO: 2078 - DNA, 25 bp, Homo sapiens
ggcatggcttgggtatctcaattcc

SEQ ID NO: 2079 - DNA, 25 bp, Homo sapiens
tctcaattcccttataaatccactg

SEQ ID NO: 2080 - DNA, 25 bp, Homo sapiens
tgcatcatcaagcacgaccacattg

SEQ ID NO: 2081 - DNA, 25 bp, Homo sapiens
acattgtttccaccatttactcaac

SEQ ID NO: 2082 - DNA, 25 bp, Homo sapiens
atcccactggcattgattttgatcc

SEQ ID NO: 2083 - DNA, 25 bp, Homo sapiens
ggaggtgaatggccaagtccttttg

SEQ ID NO: 2084 - DNA, 25 bp, Homo sapiens
atcagtttccacatcctatgtacta

SEQ ID NO: 2085 - DNA, 25 bp, Homo sapiens
aaagtctttatctgacccatcaaca

SEQ ID NO: 2086 - DNA, 25 bp, Homo sapiens
agagaacggaaagcagccactaccc

SEQ ID NO: 2087 - DNA, 25 bp, Homo sapiens
gcagccactaccctgggattaatct

SEQ ID NO: 2088 - DNA, 25 bp, Homo sapiens
taatatgttggcttcctttttttgt

SEQ ID NO: 2089 - DNA, 25 bp, Homo sapiens
gaacacatccaaaatgcatgattct

SEQ ID NO: 2090 - DNA, 25 bp, Homo sapiens
tatagatctgattctttcttttcct

SEQ ID NO: 2091 - DNA, 25 bp, Homo sapiens
aactgggattaatgtatgctctaga

SEQ ID NO: 2092 - DNA, 25 bp, Homo sapiens
tgtatgctctagatccatttattag

SEQ ID NO: 2093 - DNA, 25 bp, Homo sapiens
ataactcactcatatagctctgcct

SEQ ID NO: 2094 - DNA, 25 bp, Homo sapiens
atgtctgcttaatcagtgttaaact

SEQ ID NO: 2095 - DNA, 25 bp, Homo sapiens
ataacctgaatgttggtctctttgt

SEQ ID NO: 2096 - DNA, 25 bp, Homo sapiens
tggtctctttgtacacatcttttct

SEQ ID NO: 2097 - DNA, 25 bp, Homo sapiens
cacatcttttctatgactgcaaatc

SEQ ID NO: 2098 - DNA, 25 bp, Homo sapiens
gactgcaaatcttcactttatgtat

SEQ ID NO: 2099 - DNA, 25 bp, Homo sapiens
ctttatgtatcatttttactgtcat

SEQ ID NO: 2100 - DNA, 25 bp, Homo sapiens
aagcaccagggcacggacaggaata

SEQ ID NO: 2101 - DNA, 25 bp, Homo sapiens
gtactgaattagccactttctccat

SEQ ID NO: 2102 - DNA, 25 bp, Homo sapiens
ctttctccatagccaagttgcgaat

SEQ ID NO: 2103 - DNA, 25 bp, Homo sapiens
ggccccggcaagttggacaacatgt

SEQ ID NO: 2104 - DNA, 25 bp, Homo sapiens
gagctttgggcgacagttgctacaa

SEQ ID NO: 2105 - DNA, 25 bp, Homo sapiens
aacaagatggccactctgacattga

SEQ ID NO: 2106 - DNA, 25 bp, Homo sapiens
gactggacactcaaaaagactcgcc

SEQ ID NO: 2107 - DNA, 25 bp, Homo sapiens
attgttggatgcagttgtgccagtc

SEQ ID NO: 2108 - DNA, 25 bp, Homo sapiens
tggacacttcgaggtaccggtaggt

SEQ ID NO: 2109 - DNA, 25 bp, Homo sapiens
agcagtctgacggctcatttctgaa

SEQ ID NO: 2110 - DNA, 25 bp, Homo sapiens
gtttacatgccataagtccttttaa

SEQ ID NO: 2111 - DNA, 25 bp, Homo sapiens
ttttgctggcgtcgttggagttaaa

SEQ ID NO: 2112 - DNA, 25 bp, Homo sapiens
tggagttaaaatgccccgttactgt

SEQ ID NO: 2113 - DNA, 25 bp, Homo sapiens
aaacaatgtcactctggctaacaaa

SEQ ID NO: 2114 - DNA, 25 bp, Homo sapiens
actcaaagactgtcctggtttcgtg

SEQ ID NO: 2115 - DNA, 25 bp, Homo sapiens
tcgtgtttacccctcgatcaaggga

SEQ ID NO: 2116 - DNA, 25 bp, Homo sapiens
ttccaccaaacttccctagtgaaat

SEQ ID NO: 2117 - DNA, 25 bp, Homo sapiens
agtgaaatccccggaatctgccatt

SEQ ID NO: 2118 - DNA, 25 bp, Homo sapiens
aacaaactcaaaaccatgcttccaa

SEQ ID NO: 2119 - DNA, 25 bp, Homo sapiens
gtcacaatctttctcctgtttaaca

SEQ ID NO: 2120 - DNA, 25 bp, Homo sapiens
ctgatgaagttatgtctccccatgg

SEQ ID NO: 2121 - DNA, 25 bp, Homo sapiens
ctccccatggagaacctatcaagat

SEQ ID NO: 2122 - DNA, 25 bp, Homo sapiens
ccggcatctcttccaaggcaatggg

SEQ ID NO: 2123 - DNA, 25 bp, Homo sapiens
ggatcatgaattccttcgtcaacga

SEQ ID NO: 2124 - DNA, 25 bp, Homo sapiens
cgtcaacgacatcttcgagcgcatc

SEQ ID NO: 2125 - DNA, 25 bp, Homo sapiens
attaacgctacgatgcctgaaccta

SEQ ID NO: 2126 - DNA, 25 bp, Homo sapiens
gcattacaacaagcgctcgaccatc

SEQ ID NO: 2127 - DNA, 25 bp, Homo sapiens
tcgaccatcacctccagggagatcc

SEQ ID NO: 2128 - DNA, 25 bp, Homo sapiens
gtaagcatctttacacctaatccca

SEQ ID NO: 2129 - DNA, 25 bp, Homo sapiens
tacacctaatcccaaaggctctttt

SEQ ID NO: 2130 - DNA, 25 bp, Homo sapiens
taagagccacgcatgttttcaataa

SEQ ID NO: 2131 - DNA, 25 bp, Homo sapiens
gctcctgccccaaagaagggctcca

SEQ ID NO: 2132 - DNA, 25 bp, Homo sapiens
agggctccaagaaggcggtgactaa

SEQ ID NO: 2133 - DNA, 25 bp, Homo sapiens
ggcctgcagtggatagagcctagca

SEQ ID NO: 2134 - DNA, 25 bp, Homo sapiens
atctctgacagtgatttccagcgac

SEQ ID NO: 2135 - DNA, 25 bp, Homo sapiens
ccagcgactttgtcaacacggtccg

SEQ ID NO: 2136 - DNA, 25 bp, Homo sapiens
tccgcccccagcaagtataagagga

SEQ ID NO: 2137 - DNA, 25 bp, Homo sapiens
acaaatgtctttactgccttgtctt

SEQ ID NO: 2138 - DNA, 25 bp, Homo sapiens
cccttgccacttgtcattattcaag

SEQ ID NO: 2139 - DNA, 25 bp, Homo sapiens
ttaccagctgtgcttgcgttgcaag

SEQ ID NO: 2140 - DNA, 25 bp, Homo sapiens
cttgcgttgcaagacctgtcacagt

SEQ ID NO: 2141 - DNA, 25 bp, Homo sapiens
ctgcaccattcaaactagccaaccc

SEQ ID NO: 2142 - DNA, 25 bp, Homo sapiens
tcttcggggctcatgctaggcccga

SEQ ID NO: 2143 - DNA, 25 bp, Homo sapiens
gctaggcccgagtgcattcaataaa

SEQ ID NO: 2144 - DNA, 25 bp, Homo sapiens
gaacttcatatggcagtccatttag

SEQ ID NO: 2145 - DNA, 25 bp, Homo sapiens
taaaatctggttccttcttagcaaa

SEQ ID NO: 2146 - DNA, 25 bp, Homo sapiens
aaaactctgtgacatagtttctttt

SEQ ID NO: 2147 - DNA, 25 bp, Homo sapiens
tactccccgtatcaggtattttcga

SEQ ID NO: 2148 - DNA, 25 bp, Homo sapiens
aagtactcaagtcacatcacattca

SEQ ID NO: 2149 - DNA, 25 bp, Homo sapiens
aaacaccagcagatactattacttg

SEQ ID NO: 2150 - DNA, 25 bp, Homo sapiens
attgggagggggcacttttcatagt

SEQ ID NO: 2151 - DNA, 25 bp, Homo sapiens
ggcacttttcatagtcttggaatgc

SEQ ID NO: 2152 - DNA, 25 bp, Homo sapiens
tattatatttgatactcttacagtt

SEQ ID NO: 2153 - DNA, 25 bp, Homo sapiens
aattattgaccagttttgaagtttg

SEQ ID NO: 2154 - DNA, 25 bp, Homo sapiens
gaaggactcttgttttacacttgta

SEQ ID NO: 2155 - DNA, 25 bp, Homo sapiens
tgatctttaaattttcccacaccat

SEQ ID NO: 2156 - DNA, 25 bp, Homo sapiens
aaattttcccacaccataagagagg

SEQ ID NO: 2157 - DNA, 25 bp, Homo sapiens
aaagctatatcattcccagttatta

SEQ ID NO: 2158 - DNA, 25 bp, Homo sapiens
gttaacacaaattcagccacattct

SEQ ID NO: 2159 - DNA, 25 bp, Homo sapiens
gagtattgtttgttcacctttcaga

SEQ ID NO: 2160 - DNA, 25 bp, Homo sapiens
tgtttgttcacctttcagacttggt

SEQ ID NO: 2161 - DNA, 25 bp, Homo sapiens
gacttggtgatactggacatgtcag

SEQ ID NO: 2162 - DNA, 25 bp, Homo sapiens
aggatcttctaagtgtataactgtc

SEQ ID NO: 2163 - DNA, 25 bp, Homo sapiens
gcccatcactgtggcacactgtaga

SEQ ID NO: 2164 - DNA, 25 bp, Homo sapiens
aaagcctatgcttgtgtaagtgaaa

SEQ ID NO: 2165 - DNA, 25 bp, Homo sapiens
tagaggctcagtacttttccaatgc

SEQ ID NO: 2166 - DNA, 25 bp, Homo sapiens
gctctatacgtagtgaggacccaga

SEQ ID NO: 2167 - DNA, 25 bp, Homo sapiens
gtgaggacccagatttagagaaact

SEQ ID NO: 2168 - DNA, 25 bp, Homo sapiens
atttatctccgcatttgtgtgtgtg

SEQ ID NO: 2169 - DNA, 25 bp, Homo sapiens
aactctgtaggccaataaaccaaca

SEQ ID NO: 2170 - DNA, 25 bp, Homo sapiens
aaatagcttccagaatgtggtggtt

SEQ ID NO: 2171 - DNA, 25 bp, Homo sapiens
gaatgtggtggttctgggcaacaaa

SEQ ID NO: 2172 - DNA, 25 bp, Homo sapiens
gagattgtggcgacgtggagattaa

SEQ ID NO: 2173 - DNA, 25 bp, Homo sapiens
tgatcaagtcttgtcagttcgtgcc

SEQ ID NO: 2174 - DNA, 25 bp, Homo sapiens
tctttccccatgttccctgggaaga

SEQ ID NO: 2175 - DNA, 25 bp, Homo sapiens
gttctgtgccgcagcacgcaaaatt

SEQ ID NO: 2176 - DNA, 25 bp, Homo sapiens
gaattctacagactagctctatacg

SEQ ID NO: 2177 - DNA, 25 bp, Homo sapiens
catgtgtctctgtaatagggataat

SEQ ID NO: 2178 - DNA, 25 bp, Homo sapiens
tctatcttatgttgtcttgaggcca

SEQ ID NO: 2179 - DNA, 25 bp, Homo sapiens
gaggccaagatttaccacgtttgcc

SEQ ID NO: 2180 - DNA, 25 bp, Homo sapiens
ttaccacgtttgcccagtgtattga

SEQ ID NO: 2181 - DNA, 25 bp, Homo sapiens
ggtagaaggtagttccatgttccat

SEQ ID NO: 2182 - DNA, 25 bp, Homo sapiens
tccatgttccatttgtagatcttta

SEQ ID NO: 2183 - DNA, 25 bp, Homo sapiens
agaatgtggctcagttctggtcctt

SEQ ID NO: 2184 - DNA, 25 bp, Homo sapiens
ggtccttcaagcctgtatggtttgg

SEQ ID NO: 2185 - DNA, 25 bp, Homo sapiens
ttggattttcagtaggggacagttg

SEQ ID NO: 2186 - DNA, 25 bp, Homo sapiens
ggagtcaatctctttggtacacagg

SEQ ID NO: 2187 - DNA, 25 bp, Homo sapiens
ttcattcacgaatctcttattttgg

SEQ ID NO: 2188 - DNA, 25 bp, Homo sapiens
aatattggtacctgtcattttttca

SEQ ID NO: 2189 - DNA, 25 bp, Homo sapiens
tgttagtgactttgatgccttttaa

SEQ ID NO: 2190 - DNA, 25 bp, Homo sapiens
aaagagatctctagcgtgtgtgaat

SEQ ID NO: 2191 - DNA, 25 bp, Homo sapiens
gcgtgtgtgaatagagctccagatg

SEQ ID NO: 2192 - DNA, 25 bp, Homo sapiens
gctccagatgcctctaaaagccgca

SEQ ID NO: 2193 - DNA, 25 bp, Homo sapiens
agccgcatgtacaaaggaagccacg

SEQ ID NO: 2194 - DNA, 25 bp, Homo sapiens
aaaggaagccacgtctatcctgtct

SEQ ID NO: 2195 - DNA, 25 bp, Homo sapiens
tgcttttcctgttttgtaacctctt

SEQ ID NO: 2196 - DNA, 25 bp, Homo sapiens
ttgtaacctctttgtactttgttca

SEQ ID NO: 2197 - DNA, 25 bp, Homo sapiens
gtactttgttcatggtgacttgtaa

SEQ ID NO: 2198 - DNA, 25 bp, Homo sapiens
ggaaggggtgcctagatgcctttgt

SEQ ID NO: 2199 - DNA, 25 bp, Homo sapiens
ggcctctgaagagtcaaggtctgct

SEQ ID NO: 2200 - DNA, 25 bp, Homo sapiens
tgtgtttacctcactcaagctgaca

SEQ ID NO: 2201 - DNA, 25 bp, Homo sapiens
gggaatctatccttcttttagacac

SEQ ID NO: 2202 - DNA, 25 bp, Homo sapiens
gacacacggtaatccttgggctgta

SEQ ID NO: 2203 - DNA, 25 bp, Homo sapiens
gggctgtattactgaaggcttttta

SEQ ID NO: 2204 - DNA, 25 bp, Homo sapiens
aggtgaattcctggtcttggcagat

SEQ ID NO: 2205 - DNA, 25 bp, Homo sapiens
ggagcacagaagtcgtggcctgagg

SEQ ID NO: 2206 - DNA, 25 bp, Homo sapiens
ggcctgaggctgttctatgggcact

SEQ ID NO: 2207 - DNA, 25 bp, Homo sapiens
tgggcacttggggctaaatcgcctc

SEQ ID NO: 2208 - DNA, 25 bp, Homo sapiens
aatcgcctcctgagggtgactgttg

SEQ ID NO: 2209 - DNA, 25 bp, Homo sapiens
gtgactgttgcttattctgctggac

SEQ ID NO: 2210 - DNA, 25 bp, Homo sapiens
gaagtggtaggcaagagtctctgtg

SEQ ID NO: 2211 - DNA, 25 bp, Homo sapiens
gagtctctgtgttaccatgggaacg

SEQ ID NO: 2212 - DNA, 25 bp, Homo sapiens
gaacgattaagttttccaaggtgca

SEQ ID NO: 2213 - DNA, 25 bp, Homo sapiens
cctcattccagcttcagggtcaatg

SEQ ID NO: 2214 - DNA, 25 bp, Homo sapiens
gggtcaatgacttactagctcagag

SEQ ID NO: 2215 - DNA, 25 bp, Homo sapiens
acatacctactatctgtacagagtg

SEQ ID NO: 2216 - DNA, 25 bp, Homo sapiens
gagtgactctcattacccagagaac

SEQ ID NO: 2217 - DNA, 25 bp, Homo sapiens
ggggagtacttaaggtgtatgagca

SEQ ID NO: 2218 - DNA, 25 bp, Homo sapiens
aacaaattgttatccaggtcactcc

SEQ ID NO: 2219 - DNA, 25 bp, Homo sapiens
tcactccagaactgttgtatacaga

SEQ ID NO: 2220 - DNA, 25 bp, Homo sapiens
ttgtgccctgaaaattgtatcaaca

SEQ ID NO: 2221 - DNA, 25 bp, Homo sapiens
taaacctatttcctagcatgccttc

SEQ ID NO: 2222 - DNA, 25 bp, Homo sapiens
gttgtgccagaccctagattgtgaa

SEQ ID NO: 2223 - DNA, 25 bp, Homo sapiens
cactgttcttctgttgtacgagctc

SEQ ID NO: 2224 - DNA, 25 bp, Homo sapiens
caatgtcacatcgcttcatgggcat

SEQ ID NO: 2225 - DNA, 25 bp, Homo sapiens
ggcatggcccatggagcatctgggt

SEQ ID NO: 2226 - DNA, 25 bp, Homo sapiens
tattggctcttctgcgaggctgata

SEQ ID NO: 2227 - DNA, 25 bp, Homo sapiens
cctctcttccacatgatcatttgca

SEQ ID NO: 2228 - DNA, 25 bp, Homo sapiens
ctgcgtggatgtttccttaacctca

SEQ ID NO: 2229 - DNA, 25 bp, Homo sapiens
tgtctaatgctagttcagggcctcc

SEQ ID NO: 2230 - DNA, 25 bp, Homo sapiens
ggcctccaggcattgatttgtacag

SEQ ID NO: 2231 - DNA, 25 bp, Homo sapiens
ggtaactcccaatgaggcttctgtt

SEQ ID NO: 2232 - DNA, 25 bp, Homo sapiens
ggtgggcgtggtactgagagtccca

SEQ ID NO: 2233 - DNA, 25 bp, Homo sapiens
gtgaggggagtgccctcaggcaggc

SEQ ID NO: 2234 - DNA, 25 bp, Homo sapiens
ggagggaacagcgctgacattcagc

SEQ ID NO: 2235 - DNA, 25 bp, Homo sapiens
tcagctggttcgcactgatacggct

SEQ ID NO: 2236 - DNA, 25 bp, Homo sapiens
gatacggctcaaccagtttgttaaa

SEQ ID NO: 2237 - DNA, 25 bp, Homo sapiens
ggacttcccgctgcatttgagaagc

SEQ ID NO: 2238 - DNA, 25 bp, Homo sapiens
ttgagaagctttgcagcgccatctg

SEQ ID NO: 2239 - DNA, 25 bp, Homo sapiens
tgctttgcgccttcatcttgaagca

SEQ ID NO: 2240 - DNA, 25 bp, Homo sapiens
gaagcactctgaaattgcctgttta

SEQ ID NO: 2241 - DNA, 25 bp, Homo sapiens
gaatcatggagttgctactgcttct

SEQ ID NO: 2242 - DNA, 25 bp, Homo sapiens
agtgcattgtcgttcttgtgtcagt

SEQ ID NO: 2243 - DNA, 25 bp, Homo sapiens
aactgtgagagatgtctgggcctgc

SEQ ID NO: 2244 - DNA, 25 bp, Homo sapiens
gagatgtctgggcctgcagaagtcc

SEQ ID NO: 2245 - DNA, 25 bp, Homo sapiens
gcagaagtccagcattgctcaaaaa

SEQ ID NO: 2246 - DNA, 25 bp, Homo sapiens
attatttatccccctacattatgta

SEQ ID NO: 2247 - DNA, 25 bp, Homo sapiens
aggacattgtgtttcctgtcatgta

SEQ ID NO: 2248 - DNA, 25 bp, Homo sapiens
aaaggcatgaactcagctcctaatc

SEQ ID NO: 2249 - DNA, 25 bp, Homo sapiens
actcagctcctaatcgtcactgtat

SEQ ID NO: 2250 - DNA, 25 bp, Homo sapiens
aatcgtcactgtatagtcctgaatt

SEQ ID NO: 2251 - DNA, 25 bp, Homo sapiens
tagagttaattccctcttggaactt

SEQ ID NO: 2252 - DNA, 25 bp, Homo sapiens
tttctttgttcttcagtagttactt

SEQ ID NO: 2253 - DNA, 25 bp, Homo sapiens
aagggttgtctgtcaaacaattctt

SEQ ID NO: 2254 - DNA, 25 bp, Homo sapiens
gaaaaaagctatcagctgtatgtta

SEQ ID NO: 2255 - DNA, 25 bp, Homo sapiens
agagagactcttactaacatgttgt

SEQ ID NO: 2256 - DNA, 25 bp, Homo sapiens
attttatggtttccatgcttttgta

SEQ ID NO: 2257 - DNA, 25 bp, Homo sapiens
tccatgcttttgtaatcctaaaaat

SEQ ID NO: 2258 - DNA, 25 bp, Homo sapiens
aaaatattaatgtctagttgttcta

SEQ ID NO: 2259 - DNA, 25 bp, Homo sapiens
ttataaccacatttgcgctctatgc

SEQ ID NO: 2260 - DNA, 25 bp, Homo sapiens
cacatttgcgctctatgcaagccct

SEQ ID NO: 2261 - DNA, 25 bp, Homo sapiens
cgctctatgcaagcccttggaacag

SEQ ID NO: 2262 - DNA, 25 bp, Homo sapiens
aatttttctatggtagcctagttat

SEQ ID NO: 2263 - DNA, 25 bp, Homo sapiens
gtagcctagttatttgagcctggtt

SEQ ID NO: 2264 - DNA, 25 bp, Homo sapiens
atttgagcctggtttcaatgtgaga

SEQ ID NO: 2265 - DNA, 25 bp, Homo sapiens
gattaaacctatacaagtctggcaa

SEQ ID NO: 2266 - DNA, 25 bp, Homo sapiens
tacaagtctggcaatgagctctgca

SEQ ID NO: 2267 - DNA, 25 bp, Homo sapiens
aatgagctctgcatgaggaaatgga

SEQ ID NO: 2268 - DNA, 25 bp, Homo sapiens
tccttttctgatcatgggctctgga

SEQ ID NO: 2269 - DNA, 25 bp, Homo sapiens
gatcatgggctctggaaagtattca

SEQ ID NO: 2270 - DNA, 25 bp, Homo sapiens
gaaagtattcatggcctttaccagc

SEQ ID NO: 2271 - DNA, 25 bp, Homo sapiens
accagcattcagtataaaccagaga

SEQ ID NO: 2272 - DNA, 25 bp, Homo sapiens
atatgtacttacgtgtgtctgtgag

SEQ ID NO: 2273 - DNA, 25 bp, Homo sapiens
tgtgtgtctgagtgttattctgaac

SEQ ID NO: 2274 - DNA, 25 bp, Homo sapiens
gagtgttattctgaacagcttgtaa

SEQ ID NO: 2275 - DNA, 25 bp, Homo sapiens
aagctgagttcttttggcaaatata

SEQ ID NO: 2276 - DNA, 25 bp, Homo sapiens
atatgtttagagatgccgccagaac

SEQ ID NO: 2277 - DNA, 25 bp, Homo sapiens
agcatgttctccatttgcagtctac

SEQ ID NO: 2278 - DNA, 25 bp, Homo sapiens
gaaaatccttaccagttgtttgtca

SEQ ID NO: 2279 - DNA, 25 bp, Homo sapiens
tcttgttctcttgctggttattggc

SEQ ID NO: 2280 - DNA, 25 bp, Homo sapiens
gctggttattggcagactcagtctt

SEQ ID NO: 2281 - DNA, 25 bp, Homo sapiens
gatagggaaacccacgtatgccttt

SEQ ID NO: 2282 - DNA, 25 bp, Homo sapiens
atgcctttgaggctagggactatgt

SEQ ID NO: 2283 - DNA, 25 bp, Homo sapiens
gggactatgttgtaagttcacctgt

SEQ ID NO: 2284 - DNA, 25 bp, Homo sapiens
gttcacctgtgatggccaggtcata

SEQ ID NO: 2285 - DNA, 25 bp, Homo sapiens
agactggggacccagaggcacttgt

SEQ ID NO: 2286 - DNA, 25 bp, Homo sapiens
acttgttatgcttccacactacgaa

SEQ ID NO: 2287 - DNA, 25 bp, Homo sapiens
acttgggacgtgagttgtctctcaa

SEQ ID NO: 2288 - DNA, 25 bp, Homo sapiens
gagttgtctctcaaagcacagtagt

SEQ ID NO: 2289 - DNA, 25 bp, Homo sapiens
aagcacagctggggattgatcatgg

SEQ ID NO: 2290 - DNA, 25 bp, Homo sapiens
ggagcttggcagctctcatatccag

SEQ ID NO: 2291 - DNA, 25 bp, Homo sapiens
gcagctctcatatccagaataagcc

SEQ ID NO: 2292 - DNA, 25 bp, Homo sapiens
ataagccactaagacggaactcatc

SEQ ID NO: 2293 - DNA, 25 bp, Homo sapiens
actaagacggaactcatcaatcacc

SEQ ID NO: 2294 - DNA, 25 bp, Homo sapiens
aattaacttagcatgcaacttaccg

SEQ ID NO: 2295 - DNA, 25 bp, Homo sapiens
aactgccatatttaccagatgtttt

SEQ ID NO: 2296 - DNA, 25 bp, Homo sapiens
cagatgttttctttaaccgaacttg

SEQ ID NO: 2297 - DNA, 25 bp, Homo sapiens
ttaaccgaacttgtctgtaaatata

SEQ ID NO: 2298 - DNA, 25 bp, Homo sapiens
gatagcgaatgcactcagggtcagc

SEQ ID NO: 2299 - DNA, 25 bp, Homo sapiens
acttatttaaatgacagcacctgag

SEQ ID NO: 2300 - DNA, 25 bp, Homo sapiens
agaggaaccgttttacactggatgt

SEQ ID NO: 2301 - DNA, 25 bp, Homo sapiens
tacatgtctgttgttggtcatctct

SEQ ID NO: 2302 - DNA, 25 bp, Homo sapiens
gtcatctctcctgtgtcttaaatac

SEQ ID NO: 2303 - DNA, 25 bp, Homo sapiens
gagcatagtgtttgggctagtgggt

SEQ ID NO: 2304 - DNA, 25 bp, Homo sapiens
gctagtgggtttctgacagcccatg

SEQ ID NO: 2305 - DNA, 25 bp, Homo sapiens
acagcccatgggaatgccctgaaac

SEQ ID NO: 2306 - DNA, 25 bp, Homo sapiens
ggaatgccctgaaactactgtatct

SEQ ID NO: 2307 - DNA, 25 bp, Homo sapiens
gatgtttgttttcgatgaggttcca

SEQ ID NO: 2308 - DNA, 25 bp, Homo sapiens
cgatgaggttccatgttttgttttc

SEQ ID NO: 2309 - DNA, 25 bp, Homo sapiens
ttggactagtcctatcataaatggg

SEQ ID NO: 2310 - DNA, 25 bp, Homo sapiens
gatactgtaccatttgcatgtgtgc

SEQ ID NO: 2311 - DNA, 25 bp, Homo sapiens
tgtgtttgtgtctttctgcaggcac

SEQ ID NO: 2312 - DNA, 25 bp, Homo sapiens
tgtgtctttctgcaggcacatctca

SEQ ID NO: 2313 - DNA, 25 bp, Homo sapiens
atcacttttgtgataggctcacttt

SEQ ID NO: 2314 - DNA, 25 bp, Homo sapiens
ggctcacttttgtgaatgatctgag

SEQ ID NO: 2315 - DNA, 25 bp, Homo sapiens
gtttgaaagatctagttgcatacac

SEQ ID NO: 2316 - DNA, 25 bp, Homo sapiens
ttgcatacacagactcttggatcaa

SEQ ID NO: 2317 - DNA, 25 bp, Homo sapiens
ctctgggctcacttcttagatcagt

SEQ ID NO: 2318 - DNA, 25 bp, Homo sapiens
acttcttagatcagtctgtggccaa

SEQ ID NO: 2319 - DNA, 25 bp, Homo sapiens
aattcctggcacatcagtttgtcaa

SEQ ID NO: 2320 - DNA, 25 bp, Homo sapiens
aagacacttcttccaaaccttgaat

SEQ ID NO: 2321 - DNA, 25 bp, Homo sapiens
gatgtgtgtttacttcatgtttaca

SEQ ID NO: 2322 - DNA, 25 bp, Homo sapiens
atcagccaaaaccataacttacaat

SEQ ID NO: 2323 - DNA, 25 bp, Homo sapiens
ttggatatgctttaccattcttagg

SEQ ID NO: 2324 - DNA, 25 bp, Homo sapiens
accattcttaggtttctgtggaaca

SEQ ID NO: 2325 - DNA, 25 bp, Homo sapiens
tttttccaattgctattgcccaaga

SEQ ID NO: 2326 - DNA, 25 bp, Homo sapiens
gctattgcccaagaattgctttcca

SEQ ID NO: 2327 - DNA, 25 bp, Homo sapiens
gaattgctttccatgcacatattgt

SEQ ID NO: 2328 - DNA, 25 bp, Homo sapiens
ttgtaaaaattccgctttgtgccac

SEQ ID NO: 2329 - DNA, 25 bp, Homo sapiens
gctttgtgccacaggtcatgattgt

SEQ ID NO: 2330 - DNA, 25 bp, Homo sapiens
agggactatttgtattgtatgttgc

SEQ ID NO: 2331 - DNA, 25 bp, Homo sapiens
acaatcggctaaccttgacatttct

SEQ ID NO: 2332 - DNA, 25 bp, Homo sapiens
catatgccactatctcggtagttca

SEQ ID NO: 2333 - DNA, 25 bp, Homo sapiens
taaattgccttgaagtttaccttgt

SEQ ID NO: 2334 - DNA, 25 bp, Homo sapiens
ccttgtgctggagagccttatgata

SEQ ID NO: 2335 - DNA, 25 bp, Homo sapiens
gataactccaaagactttcttacgg

SEQ ID NO: 2336 - DNA, 25 bp, Homo sapiens
taggattgtgtttcttagtcactga

SEQ ID NO: 2337 - DNA, 25 bp, Homo sapiens
atacctaaacatttctgaacatcag

SEQ ID NO: 2338 - DNA, 25 bp, Homo sapiens
tctgaacatcagtattgcagttgtg

SEQ ID NO: 2339 - DNA, 25 bp, Homo sapiens
ggaggatacatttgtttgtgttgct

SEQ ID NO: 2340 - DNA, 25 bp, Homo sapiens
aaaattccaccttgcatttgcatca

SEQ ID NO: 2341 - DNA, 25 bp, Homo sapiens
ccctcaattgaggcagttttctttg

SEQ ID NO: 2342 - DNA, 25 bp, Homo sapiens
tcagtatttttattcgccttctaga

SEQ ID NO: 2343 - DNA, 25 bp, Homo sapiens
atccacacatcacccatttatatta

SEQ ID NO: 2344 - DNA, 25 bp, Homo sapiens
ggcttaccttctgtcatcaagtgat

SEQ ID NO: 2345 - DNA, 25 bp, Homo sapiens
gtatcatcctggatcgtcatttcca

SEQ ID NO: 2346 - DNA, 25 bp, Homo sapiens
gtcatttccaaggaactagcctttc

SEQ ID NO: 2347 - DNA, 25 bp, Homo sapiens
ctttcttttcctaagcgtctgtatg

SEQ ID NO: 2348 - DNA, 25 bp, Homo sapiens
gtatgtgttctaaaacttccagtat

SEQ ID NO: 2349 - DNA, 25 bp, Homo sapiens
ctggagtacctatgtttgttttctt

SEQ ID NO: 2350 - DNA, 25 bp, Homo sapiens
gattgtttcctggtctgtgttttta

SEQ ID NO: 2351 - DNA, 25 bp, Homo sapiens
tttccttcagttttcctcatgaaga

SEQ ID NO: 2352 - DNA, 25 bp, Homo sapiens
atcacattggttgtactctgaagac

SEQ ID NO: 2353 - DNA, 25 bp, Homo sapiens
gcatttttccaacattgaaggtatt

SEQ ID NO: 2354 - DNA, 25 bp, Homo sapiens
gtcactaagagattcattcttttat

SEQ ID NO: 2355 - DNA, 25 bp, Homo sapiens
aaaagtttcactctctttatagtgc

SEQ ID NO: 2356 - DNA, 25 bp, Homo sapiens
gtgcttcaggatacaactttttcag

SEQ ID NO: 2357 - DNA, 25 bp, Homo sapiens
gatacaactttttcagggccttatt

SEQ ID NO: 2358 - DNA, 25 bp, Homo sapiens
actgattcacatgttattcttctaa

SEQ ID NO: 2359 - DNA, 25 bp, Homo sapiens
agatatggttccaggcagacctcct

SEQ ID NO: 2360 - DNA, 25 bp, Homo sapiens
ttccaggcagacctccttagagacc

SEQ ID NO: 2361 - DNA, 25 bp, Homo sapiens
atttcattactgttactgggtgcca

SEQ ID NO: 2362 - DNA, 25 bp, Homo sapiens
gggtgccaagtgtctttcatttgga

SEQ ID NO: 2363 - DNA, 25 bp, Homo sapiens
ggaagtgaacttactccagttattg

SEQ ID NO: 2364 - DNA, 25 bp, Homo sapiens
ccaaatcaaaacaccctctgtcatc

SEQ ID NO: 2365 - DNA, 25 bp, Homo sapiens
gccagttggagtttgtgctatgcag

SEQ ID NO: 2366 - DNA, 25 bp, Homo sapiens
ggatctcatcagcgtgcaaacctag

SEQ ID NO: 2367 - DNA, 25 bp, Homo sapiens
gtgcaaacctagcatcttctgtggc

SEQ ID NO: 2368 - DNA, 25 bp, Homo sapiens
ccacaagccacacacttgctttttt

SEQ ID NO: 2369 - DNA, 25 bp, Homo sapiens
cctgggtttctgtctaactcgaagt

SEQ ID NO: 2370 - DNA, 25 bp, Homo sapiens
tgtatcgggtttttttgccactggc

SEQ ID NO: 2371 - DNA, 25 bp, Homo sapiens
ggcaagaacatgccctctgtgctaa

SEQ ID NO: 2372 - DNA, 25 bp, Homo sapiens
actcgaagtcttgaatcctagctag

SEQ ID NO: 2373 - DNA, 25 bp, Homo sapiens
ctgtgctaagccaggcctgggtgtc

SEQ ID NO: 2374 - DNA, 25 bp, Homo sapiens
gtagcaaagttgatctctccatgtc

SEQ ID NO: 2375 - DNA, 25 bp, Homo sapiens
atttttctgcaggggtacacccaca

SEQ ID NO: 2376 - DNA, 25 bp, Homo sapiens
gggtacacccacatctattgtatta

SEQ ID NO: 2377 - DNA, 25 bp, Homo sapiens
ttttctctggttgatcgggatgcat

SEQ ID NO: 2378 - DNA, 25 bp, Homo sapiens
gatcgggatgcattatccaccagaa

SEQ ID NO: 2379 - DNA, 25 bp, Homo sapiens
aaaacactgtagacgactcactcac

SEQ ID NO: 2380 - DNA, 25 bp, Homo sapiens
atcaagtcttatgagccaggtgcag

SEQ ID NO: 2381 - DNA, 25 bp, Homo sapiens
gagggtggagtgtgatatgatcgtc

SEQ ID NO: 2382 - DNA, 25 bp, Homo sapiens
agcactgcatcctggacaagatagg

SEQ ID NO: 2383 - DNA, 25 bp, Homo sapiens
actgcattgtacattcattgaggac

SEQ ID NO: 2384 - DNA, 25 bp, Homo sapiens
gaggacagggactttaaacttcatt

SEQ ID NO: 2385 - DNA, 25 bp, Homo sapiens
acttcattatattgctgttgctgtg

SEQ ID NO: 2386 - DNA, 25 bp, Homo sapiens
taataccttaggttaaggccacata

SEQ ID NO: 2387 - DNA, 25 bp, Homo sapiens
gccttttctgcggaggactctgaag

SEQ ID NO: 2388 - DNA, 25 bp, Homo sapiens
ggaggactctgaagggatactaaac

SEQ ID NO: 2389 - DNA, 25 bp, Homo sapiens
tacttttacctacattgtctcttat

SEQ ID NO: 2390 - DNA, 25 bp, Homo sapiens
gaaagtgtttactatggactgaatt

SEQ ID NO: 2391 - DNA, 25 bp, Homo sapiens
tcatatattgaagccataaacccca

SEQ ID NO: 2392 - DNA, 25 bp, Homo sapiens
taaaccccaatatgactctattcct

SEQ ID NO: 2393 - DNA, 25 bp, Homo sapiens
gactctattcctagacaggacttat

SEQ ID NO: 2394 - DNA, 25 bp, Homo sapiens
ggtcattaggatgggttcctaactg

SEQ ID NO: 2395 - DNA, 25 bp, Homo sapiens
atgtttcttgttagccatgacccta

SEQ ID NO: 2396 - DNA, 25 bp, Homo sapiens
cttgttagccatgaccctataagaa

SEQ ID NO: 2397 - DNA, 25 bp, Homo sapiens
gactcagattgtatgtctctaagaa

SEQ ID NO: 2398 - DNA, 25 bp, Homo sapiens
ttctctttctctttgcagatttcta

SEQ ID NO: 2399 - DNA, 25 bp, Homo sapiens
tgcagatttctaggccgcttctgct

SEQ ID NO: 2400 - DNA, 25 bp, Homo sapiens
cttttctatagttcatgttttcttt

SEQ ID NO: 2401 - DNA, 25 bp, Homo sapiens
aagaatcttaagctttggcattaaa

SEQ ID NO: 2402 - DNA, 25 bp, Homo sapiens
gctttggcattaaatagtcctcgat

SEQ ID NO: 2403 - DNA, 25 bp, Homo sapiens
agtcctcgattcaaatctaagctca

SEQ ID NO: 2404 - DNA, 25 bp, Homo sapiens
taagctcaacatctgattaacttca

SEQ ID NO: 2405 - DNA, 25 bp, Homo sapiens
ggaaagctcttatggttcttgtcac

SEQ ID NO: 2406 - DNA, 25 bp, Homo sapiens
gctcttatggttcttgtcacctaag

SEQ ID NO: 2407 - DNA, 25 bp, Homo sapiens
gagaccatctagtaaatgacctcat

SEQ ID NO: 2408 - DNA, 25 bp, Homo sapiens
gaggcacaggtcattctttttgaac

SEQ ID NO: 2409 - DNA, 25 bp, Homo sapiens
aaacttcagtgccatggacatgatt

SEQ ID NO: 2410 - DNA, 25 bp, Homo sapiens
gagactacagcagtgttacctgtgc

SEQ ID NO: 2411 - DNA, 25 bp, Homo sapiens
acaacttactacttctgttaccttg

SEQ ID NO: 2412 - DNA, 25 bp, Homo sapiens
agtgctctaccgaatgatgctgctt

SEQ ID NO: 2413 - DNA, 25 bp, Homo sapiens
gacattttgctagcttttttcatct

SEQ ID NO: 2414 - DNA, 25 bp, Homo sapiens
cttttttcatcttagcttgtgtttt

SEQ ID NO: 2415 - DNA, 25 bp, Homo sapiens
gaatactacagcttttatcagtcag

SEQ ID NO: 2416 - DNA, 25 bp, Homo sapiens
aactgcctcaatttgtaacacttcc

SEQ ID NO: 2417 - DNA, 25 bp, Homo sapiens
taacacttccccaaattctctagaa

SEQ ID NO: 2418 - DNA, 25 bp, Homo sapiens
attctctagaaagtcctggcttgga

SEQ ID NO: 2419 - DNA, 25 bp, Homo sapiens
gctgtggctttaccttgttgtggaa

SEQ ID NO: 2420 - DNA, 25 bp, Homo sapiens
ggaagttgggttcggacaccaggat

SEQ ID NO: 2421 - DNA, 25 bp, Homo sapiens
ataatagaatcttcctctcatttcc

SEQ ID NO: 2422 - DNA, 25 bp, Homo sapiens
ttcccccagatccttgacagtataa

SEQ ID NO: 2423 - DNA, 25 bp, Homo sapiens
ggaattgcataccttggttttcagg

SEQ ID NO: 2424 - DNA, 25 bp, Homo sapiens
ggaagtccaggagtcgcgtggattt

SEQ ID NO: 2425 - DNA, 25 bp, Homo sapiens
actgtaatacttctcttggtactgt

SEQ ID NO: 2426 - DNA, 25 bp, Homo sapiens
agctcagattgtctagttgggcact

SEQ ID NO: 2427 - DNA, 25 bp, Homo sapiens
gggcactgactttcagcacattgtc

SEQ ID NO: 2428 - DNA, 25 bp, Homo sapiens
gtctcatgagacactacctcttaat

SEQ ID NO: 2429 - DNA, 25 bp, Homo sapiens
gagtagcatggccatttgtttattt

SEQ ID NO: 2430 - DNA, 25 bp, Homo sapiens
catttattttcacatgattaactga

SEQ ID NO: 2431 - DNA, 25 bp, Homo sapiens
aaatctaggttgtctatccagtatg

SEQ ID NO: 2432 - DNA, 25 bp, Homo sapiens
gtctatccagtatgtgaatgcttaa

SEQ ID NO: 2433 - DNA, 25 bp, Homo sapiens
gagtaagtcaccaggtacacaaaac

SEQ ID NO: 2434 - DNA, 25 bp, Homo sapiens
atatattcaagttgatccatattca

SEQ ID NO: 2435 - DNA, 25 bp, Homo sapiens
aatacttcagattggtcctttgtcc

SEQ ID NO: 2436 - DNA, 25 bp, Homo sapiens
tggtcctttgtccacatttgtttaa

SEQ ID NO: 2437 - DNA, 25 bp, Homo sapiens
atccttggctaaattcacatgtatc

SEQ ID NO: 2438 - DNA, 25 bp, Homo sapiens
tatacttttggattgtgcctttgtc

SEQ ID NO: 2439 - DNA, 25 bp, Homo sapiens
ttttggattgtgcctttgtcatgag

SEQ ID NO: 2440 - DNA, 25 bp, Homo sapiens
agctgagttactgaattctataagg

SEQ ID NO: 2441 - DNA, 25 bp, Homo sapiens
gtatgtagcactttcctatatattt

SEQ ID NO: 2442 - DNA, 25 bp, Homo sapiens
tgaaaactggactgggtataactat

SEQ ID NO: 2443 - DNA, 25 bp, Homo sapiens
aaaaggcacaatggtactacagaat

SEQ ID NO: 2444 - DNA, 25 bp, Homo sapiens
gttttctgttctacaaagttgatgc

SEQ ID NO: 2445 - DNA, 25 bp, Homo sapiens
gaatcagattccctatgtaaagcag

SEQ ID NO: 2446 - DNA, 25 bp, Homo sapiens
ggaattcaatgttcagtgctcaggt

SEQ ID NO: 2447 - DNA, 25 bp, Homo sapiens
tgtagtaagtactgtagtcctgtgg

SEQ ID NO: 2448 - DNA, 25 bp, Homo sapiens
gtagtcctgtgggggcaaatgtgta

SEQ ID NO: 2449 - DNA, 25 bp, Homo sapiens
ggtctaacataatgccagttccact

SEQ ID NO: 2450 - DNA, 25 bp, Homo sapiens
atgccagttccactttaactttgtt

SEQ ID NO: 2451 - DNA, 25 bp, Homo sapiens
gaagaatgtatgtagcactttccta

SEQ ID NO: 2452 - DNA, 25 bp, Homo sapiens
aaggaaggtcagtcagtgaatggga

SEQ ID NO: 2453 - DNA, 25 bp, Homo sapiens
gaaagggagctcctctagcatcaaa

SEQ ID NO: 2454 - DNA, 25 bp, Homo sapiens
gagctcctctagcatcaaactgtct

SEQ ID NO: 2455 - DNA, 25 bp, Homo sapiens
caaactgtctgcatgtcgagtctca

SEQ ID NO: 2456 - DNA, 25 bp, Homo sapiens
ctgcatgtcgagtctcagaaaaaca

SEQ ID NO: 2457 - DNA, 25 bp, Homo sapiens
aacaaggattcgtcagtcaacccct

SEQ ID NO: 2458 - DNA, 25 bp, Homo sapiens
cccctttctgcatgcacagtggatt

SEQ ID NO: 2459 - DNA, 25 bp, Homo sapiens
gcatgcacagtggatttagggtaaa

SEQ ID NO: 2460 - DNA, 25 bp, Homo sapiens
taaagtttatgttaccctgtctttg

SEQ ID NO: 2461 - DNA, 25 bp, Homo sapiens
aatgactcatgaacttaaggtactt

SEQ ID NO: 2462 - DNA, 25 bp, Homo sapiens
ccatagcggagaactactgagttaa

SEQ ID NO: 2463 - DNA, 25 bp, Homo sapiens
cttagcctgacagtgtcctgttctc

SEQ ID NO: 2464 - DNA, 25 bp, Homo sapiens
gaaatacacccactctcttggaata

SEQ ID NO: 2465 - DNA, 25 bp, Homo sapiens
atgacgtaccactcagttggaccct

SEQ ID NO: 2466 - DNA, 25 bp, Homo sapiens
gaccctcaagagtcactgctttgtc

SEQ ID NO: 2467 - DNA, 25 bp, Homo sapiens
cgcacgcttccatttgatgcatttg

SEQ ID NO: 2468 - DNA, 25 bp, Homo sapiens
atgtcattgtccttgagaccctaca

SEQ ID NO: 2469 - DNA, 25 bp, Homo sapiens
gagaccctacatgtgcagtttggct

SEQ ID NO: 2470 - DNA, 25 bp, Homo sapiens
tttcctgcaggcttttccatgagta

SEQ ID NO: 2471 - DNA, 25 bp, Homo sapiens
gaacaaatctgtatggcttttcccc

SEQ ID NO: 2472 - DNA, 25 bp, Homo sapiens
gtgaacttgtcctagtatgcttgcc

SEQ ID NO: 2473 - DNA, 25 bp, Homo sapiens
cttgcctcacaaacgttttagccat

SEQ ID NO: 2474 - DNA, 25 bp, Homo sapiens
ggtgatgttctctagccaaattcga

SEQ ID NO: 2475 - DNA, 25 bp, Homo sapiens
agcagtttcgcttatttgattattc

SEQ ID NO: 2476 - DNA, 25 bp, Homo sapiens
acgcattacgtgtaccagaaactgt

SEQ ID NO: 2477 - DNA, 25 bp, Homo sapiens
ggtacacttaactgtggagctgggg

SEQ ID NO: 2478 - DNA, 25 bp, Homo sapiens
acatgccgccttaagtgagttcaga

SEQ ID NO: 2479 - DNA, 25 bp, Homo sapiens
gagttcagatggcttatcttccggt

SEQ ID NO: 2480 - DNA, 25 bp, Homo sapiens
gaggcatcaagtacacaggtccgtt

SEQ ID NO: 2481 - DNA, 25 bp, Homo sapiens
acaggtccgttgtaaaccagtgtct

SEQ ID NO: 2482 - DNA, 25 bp, Homo sapiens
aaccagtgtcttaagtgctaacctt

SEQ ID NO: 2483 - DNA, 25 bp, Homo sapiens
tgctaaccttatcacatttgctatt

SEQ ID NO: 2484 - DNA, 25 bp, Homo sapiens
tgccttgtctgtacacctggattaa

SEQ ID NO: 2485 - DNA, 25 bp, Homo sapiens
gtaattgtccaaatgtaatgctgct

SEQ ID NO: 2486 - DNA, 25 bp, Homo sapiens
gctatgtatattatttgggttccag

SEQ ID NO: 2487 - DNA, 25 bp, Homo sapiens
ttctcaaaacactcagtgtccttac

SEQ ID NO: 2488 - DNA, 25 bp, Homo sapiens
gtgtccttacaactgcagctaaaat

SEQ ID NO: 2489 - DNA, 25 bp, Homo sapiens
caacctctccttgaatgtagataca

SEQ ID NO: 2490 - DNA, 25 bp, Homo sapiens
ggttttgcagtcaattctgaatgga

SEQ ID NO: 2491 - DNA, 25 bp, Homo sapiens
atgtggcttcagatcatttgaacga

SEQ ID NO: 2492 - DNA, 25 bp, Homo sapiens
gcaacattatctctctctaatctgc

SEQ ID NO: 2493 - DNA, 25 bp, Homo sapiens
atattccctagattgtgttgccact

SEQ ID NO: 2494 - DNA, 25 bp, Homo sapiens
ttgtgttgccactgtattgattctg

SEQ ID NO: 2495 - DNA, 25 bp, Homo sapiens
aatttggcttgtttatgcgtgattt

SEQ ID NO: 2496 - DNA, 25 bp, Homo sapiens
aaaattgcattggccaacttggagg

SEQ ID NO: 2497 - DNA, 25 bp, Homo sapiens
ggccaacttggaggcttcagtgtta

SEQ ID NO: 2498 - DNA, 25 bp, Homo sapiens
agaagaatgttcacttttgtcatct

SEQ ID NO: 2499 - DNA, 25 bp, Homo sapiens
gtcatctaattttacactgctcctt

SEQ ID NO: 2500 - DNA, 25 bp, Homo sapiens
acactgctccttcagcaaactgact

SEQ ID NO: 2501 - DNA, 25 bp, Homo sapiens
gagagataaccctgtttacctttag

SEQ ID NO: 2502 - DNA, 25 bp, Homo sapiens
agttgtggattcctcagtcttactc

SEQ ID NO: 2503 - DNA, 25 bp, Homo sapiens
gtcttactcccattactattggtca

SEQ ID NO: 2504 - DNA, 25 bp, Homo sapiens
tattggtcattcaacagcccatctt

SEQ ID NO: 2505 - DNA, 25 bp, Homo sapiens
gtaacctgacttttgcgccagaata

SEQ ID NO: 2506 - DNA, 25 bp, Homo sapiens
agttaaccacttaaacttgtcatat

SEQ ID NO: 2507 - DNA, 25 bp, Homo sapiens
ttagaaaactactcggatgctccaa

SEQ ID NO: 2508 - DNA, 25 bp, Homo sapiens
ctactcggatgctccaatgacacca

SEQ ID NO: 2509 - DNA, 25 bp, Homo sapiens
caatgacaccaaaacagattctgca

SEQ ID NO: 2510 - DNA, 25 bp, Homo sapiens
tctcgcatgcctcaatgctatgcta

SEQ ID NO: 2511 - DNA, 25 bp, Homo sapiens
cgcatgcctcaatgctatgctacat

SEQ ID NO: 2512 - DNA, 25 bp, Homo sapiens
gcctcaatgctatgctacattccaa

SEQ ID NO: 2513 - DNA, 25 bp, Homo sapiens
caatgctatgctacattccaattca

SEQ ID NO: 2514 - DNA, 25 bp, Homo sapiens
tgttttataaactgcctggccgaat

SEQ ID NO: 2515 - DNA, 25 bp, Homo sapiens
ataaactgcctggccgaatcagcct

SEQ ID NO: 2516 - DNA, 25 bp, Homo sapiens
atcagccttttcacgctcaaggtgt

SEQ ID NO: 2517 - DNA, 25 bp, Homo sapiens
gccttttcacgctcaaggtgtgagc

SEQ ID NO: 2518 - DNA, 25 bp, Homo sapiens
gttataatctcttcctagctaatgg

SEQ ID NO: 2519 - DNA, 25 bp, Homo sapiens
ctcttcctagctaatgggcttactc

SEQ ID NO: 2520 - DNA, 25 bp, Homo sapiens
cttcctagctaatgggcttactcaa

SEQ ID NO: 2521 - DNA, 25 bp, Homo sapiens
tagctaatgggcttactcaaagatt

SEQ ID NO: 2522 - DNA, 25 bp, Homo sapiens
tgggcttactcaaagattcaccacc

SEQ ID NO: 2523 - DNA, 25 bp, Homo sapiens
ctagcaatgatattctcagttgttt

SEQ ID NO: 2524 - DNA, 25 bp, Homo sapiens
agcaatgatattctcagttgtttct

SEQ ID NO: 2525 - DNA, 25 bp, Homo sapiens
gcaatgatattctcagttgtttctc

SEQ ID NO: 2526 - DNA, 25 bp, Homo sapiens
ctcagttgtttctctcttgtggtgc

SEQ ID NO: 2527 - DNA, 25 bp, Homo sapiens
ttctctcttgtggtgcagagttgca

SEQ ID NO: 2528 - DNA, 25 bp, Homo sapiens
tctcttgtggtgcagagttgcattg

SEQ ID NO: 2529 - DNA, 25 bp, Homo sapiens
ctcttgtggtgcagagttgcattgg

SEQ ID NO: 2530 - DNA, 25 bp, Homo sapiens
tgcagagttgcattgggttttctac

SEQ ID NO: 2531 - DNA, 25 bp, Homo sapiens
tgcattgggttttctacattttccc

SEQ ID NO: 2532 - DNA, 25 bp, Homo sapiens
gcattgggttttctacattttccca

SEQ ID NO: 2533 - DNA, 25 bp, Homo sapiens
cccactgagtcttccctgttgtaaa

(19) United States
(12) Patent Application Publication
(10) Pub. No.: US 2012/0237488 A1
(43) Pub. Date: Sep. 20, 2012

(54) LSC AND HSC SIGNATURES FOR PREDICTING SURVIVAL OF PATIENTS HAVING HEMATOLOGICAL CANCER

(75) Inventors: John Dick, Toronto (CA); Kolja Eppert, Toronto (CA); Igor Jurisica, Toronto (CA); Levi David Waldron, Jamaica Plain, MA (US); Mark Minden, Toronto (CA); Eric Lechman, Toronto (CA); Bjorn Nilsson, Lund (SE); Benjamin Levine Ebert, Brookline, MA (US); Katsuto Takenaka, Fukuoka (JP); Jayne S. Danska, Toronto (CA)

(73) Assignees: UNIVERSITY HEALTH NETWORK, Toronto, ON (CA); THE BRIGHAM AND WOMEN'S HOSPITAL, INC., Boston, MA (US); THE HOSPITAL FOR SICK CHILDREN, Toronto, ON (CA)

(21) Appl. No.: 13/513,268

(22) PCT Filed: Dec. 3, 2010
(86) PCT No.: PCT/CA10/02048

§ 371 Date: Jun. 1, 2012

Related U.S. Application Data
(60) Provisional application No. 61/266,704, filed on Dec. 4, 2009.

(51) Int. Cl. C40B 30/04; A61K 35/12; G06F 19/24; C40B 40/06

(52) U.S. Cl. 424/93.7; 506/9; 506/16; 702/19

(57) ABSTRACT

A method for determining prognosis in a subject having a hematological cancer comprising: a) determining an expression profile by measuring the gene expression levels of a set of genes selected from a leukemic stem cell (LSC) gene signature marker set or an hematopoietic stem cell (HSC) gene signature marker set, in a sample from a subject; and b) classifying the subject as having a good prognosis or a poor prognosis based on the expression profile; wherein a good prognosis predicts an increased likelihood of survival within a predetermined period after initial diagnosis and poor prognosis predicts a decreased likelihood of survival within the predetermined period after initial diagnosis.

### RELATED APPLICATIONS

This is a Patent Cooperation Treaty Application which claims the benefit of 35 U.S.C. 119 based on the priority of corresponding U.S. Provisional Patent Application No. 61/266,704 filed Dec. 4, 2009, which is incorporated herein in its entirety.

### FIELD OF THE DISCLOSURE

The disclosure pertains to methods and compositions for determining gene expression signatures for predicting survival in patients having a hematological malignancy and particularly leukemia patients such as AML patients.

### BACKGROUND OF THE DISCLOSURE

Acute myeloid leukemia (AML) is a clonal disease, marked by the growth of abnormally differentiated immature myeloid cells, with a long term survival rate in adult patients of only 30%^{1, 2}. The first explicit experimental evidence for the existence of leukemic stem cells (LSC), the only cell capable of initiating and sustaining the leukemic clonal disease, has been demonstrated³. Leukemia stem cells (LSCs) are a biologically distinct blast population positioned at the apex of the acute myeloid leukemia (AML) developmental hierarchy. A more complete understanding of the unique properties of LSCs is crucial for the identification of novel AML regulatory pathways and the subsequent development of innovative therapies that effectively target these cells in leukemia patients. Typically, studies overlook the heterogeneity of AML and the existence of LSC, potentially masking important molecular pathways.

While the cancer stem cell model was proposed over three decades ago, only recently has experimental evidence confirmed the hierarchical model for leukemia³. Using a quantitative assay for transplantation of primary AML into SCID or NOD/SCID mice, human AML cells that can initiate a human leukemic graft in mice (termed SCID Leukemia-Initiating Cells—SL-IC) were identified and prospectively purified³. The cells presenting with surface markers CD34⁺CD38⁻, representing from 0.1-1% of the AML cell population, were the only AML fraction capable of serially transplanting the leukemia. Additionally, this fraction could recapitulate the cellular diversity of the original leukemia, and therefore contained the LSC. The CD34⁺CD38⁺ fraction contained progenitor cells (cells capable of forming colonies but with limited self-renewal ability) while the other two fractions contain blast cells with no self-renewal capacity. Several groups have since used the NOD/SCID xenotransplant model to isolate rare cancer stem cell (CSC) in, for example, brain and breast tumours, indicating that the CSC model applies to multiple types of cancer⁴⁻⁶.

Since AML samples are more variable than normal hematopoietic cells it is essential to validate each sorted fraction. Incorrectly labeling a sorted AML fraction would severely compromise the ability to properly analyze the global gene expression data. Currently, the in vivo transplantation assay is the best technique to accurately detect LSCs. In vitro methods suffer from the alteration of marker expression and the inability to maintain LSC in culture. Importantly, a novel and improved in vivo SCID leukemia initiating cell assay to confirm the presence of LSC activity in each sorted fraction of 16 AML involving intrafemoral injection into NOD/SCID mice depleted of CD122 cells has been applied. With this assay, LSC were detected in the expected CD34+/CD38− population of sorted AML. However, in the majority of AML samples, LSC were detected in at least one additional fraction, demonstrating the critical importance of functional validation when interpreting global gene expression profiles of sorted stem cell populations¹⁹.

Significantly, while it is expected that HSC and LSC share similar regulatory pathways, a recent finding has highlighted differences between HSC and LSC regulatory networks^{7, 8}. Deletion of the tumour suppressor gene Pten in murine hematopoietic cells resulted in the generation of transplantable leukemias. However, Pten deletion in HSCs lead to HSC depletion, indicating that, unlike LSCs, HSCs could not be maintained without Pten. Regulatory differences between HSC and LSC represent a vulnerability that can be used to specifically target LSCs for eradication, leaving HSCs unharmed. Greater understanding of both LSC and HSC regulation may reveal further differences between LSC and HSC control and lead to novel therapies.

Little is currently known of the expression profile of LSC enriched sub-populations in AML. Gal et al. examined the expression of CD34+/CD38− vs CD34+/CD38+ populations in 5 AML and identified 409 genes that are 2-fold over or under expressed between the cell populations⁹. However, the different cell populations were not functionally validated, and it is likely that the CD34+/CD38+ fractions also contain LSC, therefore the gene profile is cell marker dependent, not functionally dependent. Additionally, Majeti et al. identified 3005 differentially expressed genes in a comparison between AML CD34+/CD38− cells and normal bone marrow CD34+/CD38− cells. However, the analysis did not include mature cell populations, suggesting that the profile is a leukemia specific profile, not necessarily a stem cell profile¹⁰. The prognostic significance of these profiles was not explored.

AML is a genetically heterogeneous disease, with the karyotype of the AML blast as the most important prognostic factor^{11, 12}. However, approximately half of all adult AML are cytogenetically normal at diagnosis. Within the cytogenetically normal AML (CN-AML) patient population, the mutational status of genes such as FLT3, NPM1, MN1 and CEBPA are associated with outcome; however, the association is not absolute and not all CN-AML present with such mutations, indicating that this class of AML is heterogeneous and additional factors are prognostically significant^{13, 14}. Two groups have attempted to use gene expression profiling to predict outcome specifically in CN-AML patients. Bullinger et al. developed a signature that was validated by Radmacher et al., where there was a correlation with overall survival (p=0.001) of an classification rule developed using the previously identified signature^{15, 16}. Metzeler et al. used an cohort of 163 CN-AML to develop an 86 probe signature that predicts survival in CN-AML, with a significant prediction of overall survival in an independent set of 79 CN-AML (p=0.002)¹⁷. There was a correlation with FLT3ITD status for these signatures; however, the 86 probe signature maintained association with outcome, independent of FLT3ITD status, indicating that gene expression profiling can be of value for predicting prognosis, in addition to mutational status.

### SUMMARY OF THE DISCLOSURE

A method for determining a prognosis of a subject having a hematological cancer comprising:

a) determining a gene expression level for each of a set of genes selected from leukemia stem cell (LSC) signature genes listed in Tables 2, 6, and/or 12, hematopoietic stem cell (HSC) signature genes listed in Tables 4 and/or 14, and/or CE-HSC/LSC signature genes listed in Table 19, to obtain a subject expression profile of a sample obtained from the subject; and

b) classifying the subject as having a good prognosis or a poor prognosis based on the subject expression profile;

wherein a good prognosis predicts an increased likelihood of survival within a predetermined period after initial diagnosis and poor prognosis predicts a decreased likelihood of survival within the predetermined period after initial diagnosis.

A computer-implemented method for determining a prognosis of a subject having a hematological cancer comprising: obtaining a subject expression profile and classifying, on a computer, the subject as having a good prognosis or a poor prognosis based on the subject expression profile comprising measurements of expression levels of a set of genes in a sample from the subject, wherein the set of genes is selected from genes listed in Table 2, 4, 6, 12 and 14, comprises at least 2 genes; wherein a good prognosis predicts an increased likelihood of survival within a predetermined period after initial diagnosis, and wherein a poor prognosis predicts a decreased likelihood of survival within the predetermined period after initial diagnosis.

A method for monitoring a response to a cancer treatment in a subject having a hematological cancer, comprising:

a) collecting a first sample from the subject before the subject has received the cancer treatment;

b) collecting a subsequent sample from the subject after the subject has received the cancer treatment;

c) determining the gene expression levels of a set of genes selected from LSC signature genes and/or HSC signature genes in the first and the subsequent samples according to a method described herein, to obtain a first sample subject expression profile and a subsequent sample subject expression profile, wherein the set of genes comprises at least 2 genes; and

d) calculating a first sample subject expression profile score and a subsequent sample subject expression profile score;

wherein a lower subsequent sample expression profile score compared to the first sample expression profile score is indicative of a positive response, and a higher subsequent sample expression profile score compared to the first expression profile score is indicative of a negative response.

A method of treating a subject having a hematological cancer, comprising determining a prognosis of the subject according to a method described herein, and providing a suitable cancer treatment to the subject in need thereof according to the prognosis determined.

Use of a prognosis determined according to a method described herein, and identifying a suitable treatment for treating a subject with a hematological cancer.

A composition comprising a set of nucleic acid molecules each comprising a polynucleotide probe sequence selected from SEQ ID NO:1-2533.

An array comprising for each gene in a set of genes, the set of genes comprising at least 2 of the genes listed in Table 2, 4, 6, 12 and/or 14, one or more polynucleotide probes complementary and hybridizable to a coding sequence in the gene, for determining a prognosis according to a method described herein.

A kit for determining prognosis in a subject having a hematological cancer according to the method described herein comprising:

a) an array or composition described herein;

b) a kit control; and

c) optionally instructions for use.

A computer system comprising:

a) a database including records comprising reference expression profiles associated with clinical outcomes, each reference profile comprising the expression levels of a set of genes listed in Table 2, 4, 6, 12 and/or 14;

b) a user interface capable of receiving and/or inputting a selection of gene expression levels of a set of genes, the set comprising at least 2 genes listed in Table 2, 4, 6, 12 and/or 14 for use in comparing to the gene reference expression profiles in the database;

c) an output that displays a prediction of clinical prognosis according to the expression levels of the set of genes.

In an embodiment, the expression profile is used to calculate an subject risk score, wherein the subject is classified has having a good prognosis if the subject risk score is low and as having a poor prognosis if the expression profile is high.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

FIG. 1A Experimental Design: Sixteen AML patient samples were sorted into 4 subpopulations based upon CD34 and CD38 antibody staining and cells recovered for functional and gene expression analysis. Functional validation of the presence of SCID Leukemia Initiating Cells (SL-IC) was undertaken for each fraction of 16 of the AML samples. SL-IC is a functional readout of LSC—only LSC are known to generate long term leukemic grafts in mice. Functional validation was successful for at least 1 fraction for each of 16 AML. Generally, CD34+ and CD38− and approximately 60% of CD34+/CD38+ fractions contained SL-IC. RNA was extracted from each fraction and global gene expression was measured using Affymetrix microarrays. The mRNA expression between fractions containing SL-IC and fractions that did not contain SL-IC was compared and each mRNA probe was ranked according to correlation with SL-IC. Publicly available data for gene expression and overall survival of 160 AML was used to measure prognostic significance of the top 25 LSC probe sets that positively correlated with SL-IC¹⁷.

FIG. 1B Correlation of the 25 LSC Probe Signature with Overall Survival in CN-AML: Publicly available overall survival and expression data was analyzed¹⁷. In short, the expression of each probe set was scaled to 0 across the 160 AML patient bone marrow samples using the median value. The expression of the 25 probe sets was summed for each of the 160 bone marrow AML samples (expression score). This expression score was used to divide the 160 AML patient group into two equal sized populations of 80 patients based upon above (high expression score) or below (low expression score) median expression score of the 25 LSC probe set. The overall survival of the two groups was examined using a Kaplan-Meier plot and log-rank (Mantel-Cox) test. The 25 LSC probe set signature separated the AML patients into 2 populations with distinct outcomes (poor and good survival). The AML patients with a high expression score using the 25 LSC probe set signature had lower overall survival than the AML patients with low expression (p=0.0001; median survival of 236 days vs 999 days; hazard ratio of 2.641 with a 95% Cl of 1.763 to 3.957, computed using the Mantel-Haenszel method).

FIG. 2A Experimental Design: Three pooled cord blood samples were sorted into 3 subpopulations based upon CD34 and CD38 antibody staining and cells recovered for functional and gene expression analysis. Two cell fractions enriched for HSC, Lin-CD34+CD38− (HSC-1) and Lin-CD34+CD38lowCD36− (HSC-2), and one population enriched for progenitors, Lin-CD34+CD38+ (containing all multilineage and unilineage progenitors), were obtained. Whole CB from each pooled sample set was used as a mature cell fraction. To identify a set of genes associated with the HSC subsets, a Student's ANOVA (analysis of variance) test was performed. To reduce the incidence of false positives to <1%, Benjamini and Hochberg False Discovery Rate (FDR) was applied to the analysis. Tukey Post Hoc testing revealed that 19 differentially expressed probe sets that were over-expressed in HSC-1 compared to the other 3 groups, and 28 probe sets that were over-expressed in HSC-1 and HSC-2 compared to the other 2 groups. These probe sets were combined and duplicates removed to generate a 43 HSC probe set signature. Publicly available data for gene expression and overall survival of 160 AML was used to measure prognostic significance of this 43 HSC probe set signature¹⁷.

FIG. 2B Correlation of 43 HSC Probes Signature with Overall Survival in CN-AML: Same approach as described in FIG. 1B. The AML patients with high expression of the 43 HSC probe set signature in their bone marrow cells had lower overall survival than the AML patients with low expression (p,0.0001; median survival of 233 days vs 999 days; hazard ratio of 2.680 with a 95% Cl of 1.782 to 4.030, computed using the Mantel-Haenszel method).

FIG. 3 Example of AML Cell Sorting: Fifty three million low density peripheral blood cells from AML sample 8227 were stained with CD34 and CD38 antibodies and sorted with a BD FACSAria (Becton-Dickinson). Sorting gates were set wide to minimize contamination from other fractions. Fractionated cells were captured in 100% FCS and recovered by centrifugation. As a result, the AML patient sample was sorted into 4 subpopulations based upon CD34 and CD38 antibody staining and cells recovered for functional and gene expression analysis, including injection into the right femur of mice in the SL-IC xenotransplant assay.

FIG. 4 Example of Engraftment: Ten weeks post injection of 50,000 CD34+/CD38+ cells from AML sample 8227, the mouse was euthanized by cervical dislocation and hind leg bones removed and flushed with media to recover engrafted cells. (A) Percent human AML engraftment was assessed by flow cytometry for human CD45+ staining cells. (B) Myeloid cell marker positivity (CD33) was used to indicate that human cells are AML.

FIG. 5 Strategy of transcriptional profiling of functionally determined stem cell fractions. (A) Overview of experimental design. Cells were sorted on CD34/CD38, with representative sort gates shown for AML and cord blood. Functional validation of sorted fractions was performed in vivo and combined with gene expression profiling to generate stem cell related gene expression profiles. (B) The surface marker profiles of AML are variable. Shown are the CD34/CD38 marker profiles for 16 AML that were sorted into 4 populations and assayed for LSC.

FIG. 6 Correlation between the LSC-R and HSC-R. (A) GSEA plot showing the enrichment of the HSC-R gene signature (top) and common lineage-committed progenitor gene signature (bottom) in LSC vs non-LSC gene expression profile. (B) Heat map of the HSC-R GSEA plot from 2A (top panel) showing the core enriched HSC-R genes in the LSC expression profile (CE-HSC/LSC).

FIG. 7 The LSC-R and HSC-R gene signatures correlate with the disease outcome. 160 unsorted cytogenetically normal AML samples were divided into two populations of 80 AML by expression of the stem cell gene signatures. (A) Correlation of the LSC-R and HSC-R signatures and overall survival. The * line represent patients whose AML expressed the LSC-R (left panel) or HSC-R (right panel) signatures above the median while the ** line represent those who expressed the respective stem cell signature below the median. ‘HR’ is hazard ratio. (B) Event free survival of patients stratified by expression of the LSC-R and HSC-R, as in (A). (C) The correlation between the LSC-R signature and overall survival is not based upon a single or few genes. The y axis is the log-rank p-value of each combination of probes. The x axis is the number of probes included in the analysis, starting with the top ranked probe positively correlated with LSC followed by the addition of each next ranked probe in the LSC-R gene profile (as determined by Z-score in the LSC vs non-LSC t-test). Therefore the first point on the x axis represents the p-value of the correlation with overall survival of the top ranked LSC probe. The second point is the p-value of the combination of the top two ranked LSC-R probes. (D) An AML signature based upon phenotypic markers (CD34+/CD38− ‘stem cell’ vs CD34+/CD38+‘progenitor’) does not correlate with overall survival. The * line represent patients whose AML expressed the CD34+/CD38− gene list above the median while the ** line represent those who expressed the stem cell signature below the median.

FIG. 8 Multivariate correlation of LSC, HSC gene expression signatures and molecular risk status with overall survival in a cohort of 160 cytogenetically normal AML. Overall survival curves of 160 CN-AML divided by expression of the LSC-R (A) or HSC-R (B) signatures and molecular risk with multivariate analysis of prognostic factors below. Low molecular risk group (LMR) include NPM1mut/FLT3wt CN AML; high molecular risk (HMR) include NPM1wt or FLT3ITD positive CN AML.

FIG. 9 LSC from each AML engraft mice with similar kinetics, regardless of LSC marker profile. (A) Engraftment of AML #2, derived from LSC with different CD34/CD38 marker profiles, as detected by human CD45+CD33+ chimerism 7.5-11 weeks after injection of sorted cells. (B) Engraftment of AML #5, derived from LSC with different CD34/CD38 marker profiles, as detected by human CD45+CD33+ chimerism 8-10.5 weeks after injection of sorted cells.

FIG. 10 Representative AML sample—primary and post xenograft transplantation. (A) Differentiation marker profile for primary patient AML sample 5. (B) Sorting scheme for AML sample 5 into 4 populations based upon CD34 and CD38. (C) Both CD34+/CD38+ and CD34+/CD38− cells engrafted mice, as measured by human CD45. In each case, the differentiation marker profile is identical between chimaeric cells derived from either CD34+/CD38+ or CD34+/CD38− cells injected into mice.

FIG. 11 Properties of sorted cord blood fractions. (A) Two cell fractions enriched for HSC and one population enriched for progenitors were isolated by FACS-sorting. (B) Biological assessment of FACS-sorted cells by in vitro CFC assay with myeloid (white columns) and erythroid (black columns) colonies. (C) In vivo SRC repopulating assay. Column colour denotes cell type (black—erythroid cells, white—non-erythroid) in bone marrow of right femur (R—injected femur), left femur (L) and tibias (T).

FIG. 12 Validation of differential gene expression of 19 genes included in the HSC-R gene signature. qRT-PCR was performed on 3 populations used in the development of the HSC-R signature, including two stem cell enriched populations and one progenitor enriched population: CD34+CD38-lin− cells (HSC1), CD34+CD38loCD36-lin− (HSC2), and CD34+CD38+ (progenitor). Gene expression was normalized to that of GAPDH.

FIG. 13 Correlation between the LSC-R signature and HSC gene expression data. (A) GSEA plot showing the enrichment of the LSC-R gene signature in the HSC-R gene expression profile, comparing HSC and non-HSC. (B) Heat map of the GSEA plot showing the core enriched LSC genes in the HSC expression profile as described for (A). The populations are HSC(HSC1 and HSC2), lineage-committed progenitor (Prog) and lineage+ cells (Lin+).

FIG. 14 LSC and HSC gene expression signatures correlate with poor risk AML patients. GSEA plots showing the enrichment of (A) LSC-R FDR0.10 gene signature and (B) HSC-R FDR0.05 gene signature in 110 AML split into poor and good cytogenetic risk status. The leading edge genes are listed below. Twenty-one of the 32 leading edge HSC-R genes are enriched in LSC cell fractions and are included in the CE-HSC/LSC gene list (FIG. 2A).

FIG. 15 Correlation of LSC, HSC gene expression signatures and FLT3 status with overall survival in a cohort of 160 cytogenetically normal AML. Overall survival curves of 160 CN-AML divided by expression of the LSC-R (left panel) or HSC-R (right panel) signatures and FLT3ITD status. Multivariate analysis of prognostic factors is shown below.

FIG. 16 Schematic showing a computer system.

FIG. 17 Survival graph for expression levels of 2 LSC genes CLN5 AND NF1 showing they are significantly correlated with overall survival in the 160 AML cohort (214252_s_at and 212676_at respectively). The p value is 0.0293 and the hazard ratio is 1.53.

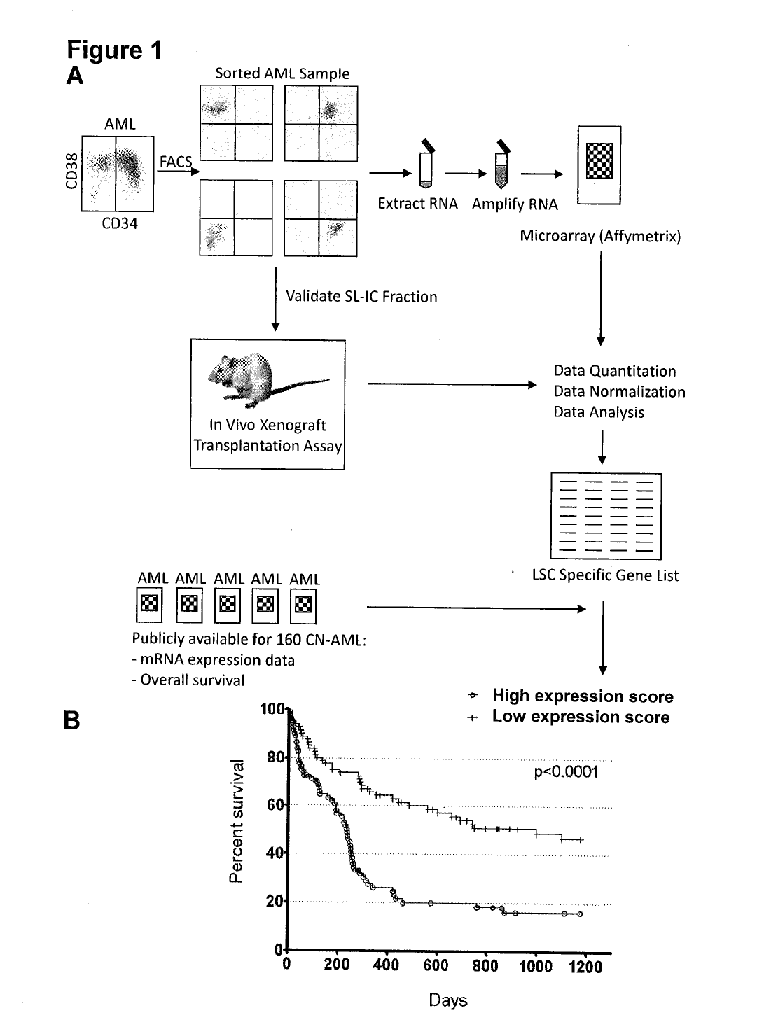

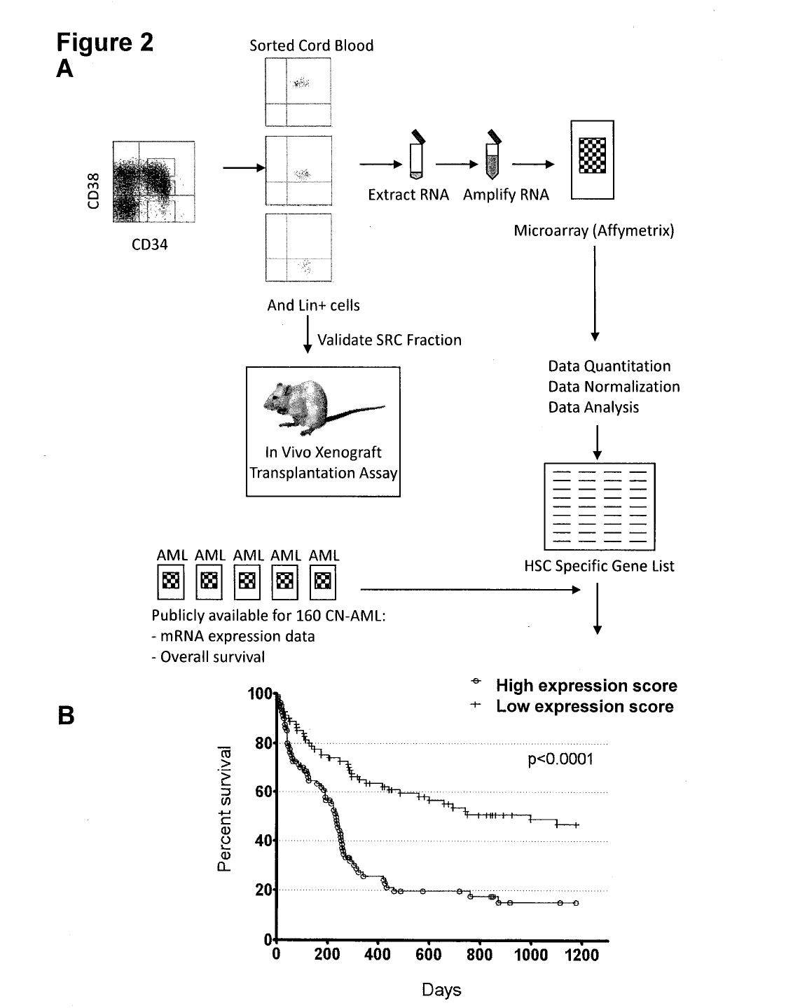

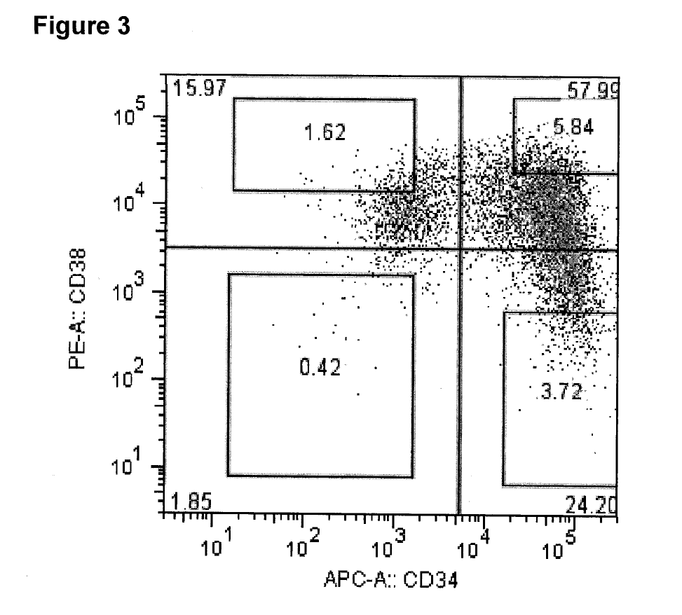

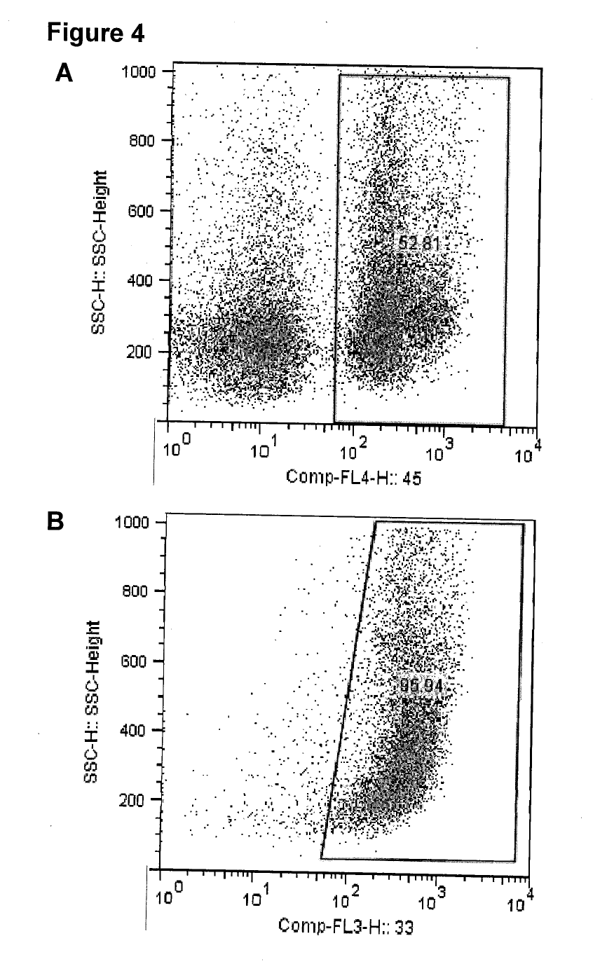

### DETAILED DESCRIPTION OF THE DISCLOSURE

### I. Definitions

As used herein, “Leukemia stem cell (LSC) signature genes” or “leukemic stem cell (LSC) signature genes includes genes listed in Tables 2, 6, and/or 12 and genes detectable by the probesets listed in Tables 1, 5 and/or 18 which are preferentially expressed in leukemic stem cells functionally defined.

As used herein, “LSC signature probe sets” as used herein refers to probesets listed for example in Tables 1, 5 and/or 18, each probeset comprising a set of probes, for example 11 probes that can be used to detect LSC signature genes.

As used herein, “Hematopoietic stem cell (HSC) signature genes” includes genes listed in Tables 4 and/or 14 and genes detectable by the probesets listed in Tables 3 and/or 17, which are preferentially expressed in hematopoietic stem cells functionally defined. Also included is the subset of HSC signature genes included in Table 20.

As used herein, “HSC signature probe sets” as used herein refers to the probesets listed for example in Tables 3 and/or 17, each probeset comprising a set of probes, for example 11 probes that can be used to detect HSC signature genes.

As used herein “core enriched HSC/LSC(CE-HSC/LSC) signature genes” refers to a subset of 44 HSC signature genes that are more highly expressed in LSC containing fractions (compared to non-LSC leukemic cells) and which are listed in Table 13 or Table 19, and which can for example detected using the corresponding probes and probesets listed for example in Tables 1, 3, 5, 17 and/or 18. These forty-four leading edge genes drive the GSEA enrichment of the HSC-R signature in the LSC gene expression data and represent HSC genes that are also differentially expressed in LSC.

As used herein “expression profile” refers to expression levels for a set of genes selected from LSC signature genes and/or HSC signature genes including for example CE-HSC/LSC signature genes. For example, an expression profile can comprise the quantitated relative expression levels of at least 2 or more genes listed in Table 2, 4 6, 12, 13, 14, 19 and/or 20 and/or genes detected by probes and probesets listed in Tables 1, 3, 5, 17 and/or 18.

A “subject expression profile” refers to the expression levels in (or corresponding to) a sample obtained from a subject. The gene expression levels can for example be used to prognose a clinical outcome based on similarity to a reference expression profile known to be associated with a particular outcome or used to calculate a subject risk score for comparison to a selected threshold.

The term “subject risk score” as used herein refers to a sum of the expression values of a set of genes selected from LSC signature genes and/or HSC signature genes (e.g. for example CE-HSC/LSC signature genes), which can be used to classify a subject. A subject risk score can be calculated for example by scaling (e.g. normalizing) each gene expression value detected for example with a probe or probeset, summing the expression values to obtain a risk score which can be compared to a reference value or standard (e.g. a threshold derived from subjects with a known outcome), where a subject risk score above the threshold predicts poor prognosis and below the threshold predicts good prognosis.

A “reference expression profile” or “reference profile” as used herein refers to the expression signature of a setset of genes (e.g. at least 2 genes LSC or HSC signature genes), associated with a clinical outcome in a patient having a hematological cancer such as a leukemia patient. The reference expression profile is identified using two or more reference patient expression profiles, wherein the expression profile is similar between reference patients with a similar outcome thereby defining an outcome class and is different to other reference expression profiles with a different outcome class. The reference expression profile is for example, a reference profile or reference signature of the expression of 2 or more, 3 or more, 4 or more or 5 or more genes listed in Table 2, 4, 6, 12, 13, 14, 19 and/or 20 and/or genes detectable with probes listed in Tables 1, 3, 5, 17 and/or 18 to which the expression levels of the corresponding genes in a patient sample are compared in methods for determining or predicting clinical outcome, e.g. good prognosis or poor prognosis. Similarly, a reference expression profile associated with good prognosis can be referred to a good prognosis reference profile and a reference expression profile associated with a poor prognosis can be referred to as a poor prognosis reference profile.

The term “classifying” as used herein refers to assigning, to a class or kind, an unclassified item. A “class” or “group” then being a grouping of items, based on one or more characteristics, attributes, properties, qualities, effects, parameters, etc., which they have in common, for the purpose of classifying them according to an established system or scheme. For example, subjects having increased expression of a set of genes selected from genes listed in Table 2, 4, 6, 12, 13, 14, 19 and/or 20 are predicted to have poor prognosis. The subject expression profile can for example be used to calculate a risk score to classify the subject, for example subjects having a summed expression value (e.g. subject risk score) above a selected threshold which can for example be the median score of a population of subjects having the same hematological cancer as the subject, can be classified as having a poor prognosis.

As used herein “prognosis” refers to an indication of the likelihood of a particular clinical outcome e.g. the resulting course of disease, for example, an indication of likelihood of survival or death due to disease within a fixed time period, and includes a “good prognosis” and a “poor prognosis”.

As used herein “outcome” or “clinical outcome” refers to the resulting course of disease and can be characterized for example by likelihood of survival or death due to disease within a fixed time period. For example a good clinical outcome includes cure, prevention of metastasis and/or survival for a fixed period of time, and a poor clinical outcome includes disease progression and/or death within a fixed period of time.

As used herein, “good prognosis” indicates that the subject is expected to survive within a set time period, for example five years of initial diagnosis of a hematological cancer such as leukemia. The set period of time varies with the disease type e.g. leukemia type and/or subtype. For example for AML, a good prognosis refers to a greater than 30%, greater than 40%, or greater than 50% chance of surviving more than 1 year, more than 2 years, more than 3 years, more than 4 years or more than 5 years after initial diagnosis. As another example, a good prognosis is used to mean an increased likelihood of survival within a predetermined time compared to a median outcome, for example the median outcome of a particular AML subtype.

As used herein, “poor prognosis” indicates that the subject is expected to die due to disease within a set time period, for example five years of initial diagnosis of a hematological cancer such as leukemia. The set period of time varies with the particular disease e.g. leukemia type and/or subtype. For example for AML, a poor prognosis refers to a less than 50%, less than 40%, or less than 30% chance of surviving greater than 1 year, greater than 2 years, greater than 3 years, greater than 4 years or greater than 5 years after initial diagnosis. As another example, a poor prognosis is used to mean a decreased likelihood of survival within a predetermined time compared for example to a median outcome, for example the median outcome of the particular hematological cancer. For example, the 5 year relative survival rates overall reported form 1999 to 2005 for ALL is 66.3% (90.9% in children under 5); for CLL is 78.8%, for AML 23.4% overall (60.2% in children under 15) and for CML 53.3% (http://www.leukemia-lymphoma.org/all_page?item_id=9346#_survival).

The term a “decreased likelihood of survival”, as used herein means an increased risk of shorter survival relative to for example the median outcome for the particular cancer. For example, increased expression of two or more genes in the gene signatures described herein can be prognostic of decreased likelihood of survival. The increased risk for example may be relative or absolute and may be expressed qualitatively or quantitatively. Examples of expressions of risk include but are not limited to, odds, probability, odds ratio, p-values, attributable risk, relative frequency, positive predictive value, negative predictive value, and relative risk.

The term an “increased likelihood of survival”, as used herein means an increased likelihood or risk of longer survival relative to a subject without the decreased expression levels. Examples of expressions of risk include but are not limited to, odds, probability, odds ratio, p-values, attributable risk, relative frequency, positive predictive value, negative predictive value, and relative risk.

As used herein “signature genes” refers to set of genes disclosed herein predicting clinical outcome in a hematological cancer subject and includes without limitation LSC-derived signature genes and/or HSC-derived signature genes as well as CE-HSC/LSC signature genes. For example, LSC signature genes includes the genes listed in Table 2, 6, and/or 12; HSC signature genes includes the genes listed in Table 4, 14 and/or 20 and CE-HSC/LSC signature genes includes genes listed in Tables 13 and 19. The gene sequences identified by accession number for example in Tables 2, 4, 6, 12, 13, 14 and 19 are herein incorporated by reference.

The term “expression level” of a gene as used herein refers to the measurable quantity of gene product produced by the gene in a sample of a patient wherein the gene product can be a transcriptional product or a translated transcriptional product. Accordingly the expression level can pertain to a nucleic acid gene product such as RNA or cDNA or a polypeptide. The expression level is derived from a subject/patient sample and/or a control sample, and can for example be detected de novo or correspond to a previous determination. The expression level can be determined or measured for example, using microarray methods, PCR methods, and/or antibody based methods, as is known to a person of skill in the art.

The term “determining an expression level” or “expression level is determined” as used in reference to a gene or (set of genes) means the application of an agent and/or method to a sample, for example a sample from the subject and/or a control sample, for ascertaining quantitatively, semi-quantitatively or qualitatively the amount of a gene expression product, for example the amount of polypeptide or mRNA. For example, a level of a gene expression can be determined by a number of methods including for example arrays and other hybridization based methods and/or PCR protocols where a probe or primer or primer set is used to ascertain the amount of nucleic acid of the gene. For example, an expression level of a gene can be determined using a probeset or one or more probes of the probeset, described herein for a particular gene. In addition more than one probeset where more than one exists, can be used to determine the expression level of the gene. Other examples include Nanostring® technology, serial analysis of gene expression (SAGE), RNA sequencing, RNase protection assays, and Northern Blot. The polypeptide level can be determined for example by immunoassay for example Western blot, flow cytometry, immunohistochemistry, ELISA, immunoprecipation and the like, where a gene or gene signature detection agent such as an antibody for example, a labeled antibody specifically binds the gene polypeptide product and permits for example relative or absolute ascertaining of the amount of polypeptide.

The term “hematological cancer” as used herein refers to cancers that affect blood and bone marrow, and include without limitation leukemia, lymphoma and multiple myeloma.

The term “CSC hematological cancer” as used herein refers to cancers that are sustained by a small population of stem-like, tumor-initiating cells

The term “leukemia” as used herein means any disease involving the progressive proliferation of abnormal leukocytes found in hemopoietic tissues, other organs and usually in the blood in increased numbers. For example, leukemia includes acute myeloid leukemia (AML), acute lymphocytic leukemia (ALL), chronic lymphocytic leukemia (CLL) and chronic myelogenous leukemia (CML) including cytogenetically normal and abnormal subtypes.

The term “lymphoma” as used herein means any disease involving the progressive proliferation of abnormal lymphoid cells. For example, lymphoma includes mantle cell lymphoma, Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas.

The term “myeloma” and/or “multiple myeloma” as used herein means any tumor or cancer composed of cells derived from the hematopoietic tissues of the bone marrow. Multiple myeloma is also knows as MM and/or plasma cell myeloma.

The term “cytogenetically normal AML” or “CN-AML” as used herein means AML or an AML cell that is characterized by normal chromosome number and structure.

The term “FLT3ITD” as used herein refers to a Fms-like tyrosine kinase 3 (FLT3) molecule (e.g. gene or protein) that comprises an internal tandem duplication (ITD). FLT3 is a receptor tyrosine kinase expressed in primitive hematopoietic cells that has been implicated in the regulation of HSC. Mutation of FLT3 is a strong prognostic indicator in CN-AML associated with poor outcome.

The term “NPM1” as used herein, refers to Nucleophosmin, including for example the sequences identified in entrez gene id 4869, herein incorporated by reference.

As used herein “sample” refers to any patient sample, including but not limited to a fluid, cell or tissue sample that comprises cancer cells such as leukemia cells including blasts, which can be assayed for gene expression levels, particularly genes differentially expressed in stem cell enriched populations or non-stem cell enriched populations, either leukemic or normal. The sample includes for example a blood sample, a fractionated blood sample, a bone marrow sample, a biopsy, a frozen tissue sample, a fresh tissue specimen, a cell sample, and/or a paraffin embedded section, material from which RNA can be extracted in sufficient quantities and with adequate quality to permit measurement of relative mRNA levels, or material from which polypeptides can be extracted in sufficient quantities and with adequate quality to permit measurement of relative polypeptide levels.

The term “sequence identity” as used herein refers to the percentage of sequence identity between two or more polypeptide sequences or two or more nucleic acid sequences that have identity or a percent identity for example about 70% identity, 80% identity, 90% identity, 95% identity, 98% identity, 99% identity or higher identity or a specified region. To determine the percent identity of two or more amino acid sequences or of two or more nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, word_length=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “subject” also referred to as “patient” as used herein refers to any member of the animal kingdom, preferably a human being.

The term “control” as used herein refers to a sample and/or an expression level or numerical value and/or range (e.g. control range) for a LSC or HSC signature gene or group of LSC or HSC signature genes, including for example CE-HSC/LSC signature genes, corresponding to their expression level in such a sample from a subject or a population of subjects (e.g. control subjects) who are known as not having or having a hematological cancer and a particular outcome. In another example, a level of expression in a sample from a subject is compared to a level of expression in a control, wherein the control comprises a control sample or a numerical value derived from a sample, optionally the same sample type as the sample (e.g. both the sample and the control are white blood cell containing fractions), from a subject known as not having or having hematological cancer and a particular outcome. Where the control is a numerical value or range, the numerical value or range is a predetermined value or range that corresponds to a level of the expression or range of levels of the genes in a group of subjects known as having a hematological cancer and outcome (e.g. threshold or cutoff level; or control range).

The term “non-cancer control” as used herein refers to a sample and/or expression level or numerical value corresponding to the expression level in a sample from a subject or a population of subjects (e.g. non-cancer control subjects) who are known as not having a hematological cancer. Similarly a “cancer” as used herein refers to a sample and/or expression level or numerical value corresponding to the expression level in a sample from a subject or a population of subjects (e.g. cancer control subjects) who are known as having a hematological cancer and a particular outcome, e.g. the same hematological cancer as the subject sample being tested e.g. both leukemias.

The term “difference in the level” as used herein when referring to a subject gene expression level in comparison to a control or previous sample refers to a measurable difference in the level or quantity of a LSC or HSC signature gene expression level or set of gene expression levels, compared to the control or previous sample that is of sufficient magnitude to indicate the subject is in a different class from the control and/or previous sample, for example a significant difference or a statistically significant difference. A difference in the level can for example be compared by calculating a subject risk score and comparing to a threshold that is for example statistically associated with a particular prognosis. A difference in a gene expression level can also be detected if a ratio of the level in a test sample as compared with a control (or previous sample) is greater than 1 or less than 1. For example, a ratio of greater than 1.5, 1.7, 2, 3, 3, 5, 10, 12, 15, 20 or more or a ratio less than 0.5, 0.25, 0.1, 0.05 or more

The term “measuring” or “measurement” as used herein refers to assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The term “set” as used herein in the context of “set of genes” means one or more, optionally 2 or more, 3 or more, 4 or more or 5 or more genes. The set can for example include genes listed in Tables 2, 4 6, 12, 13, 14, 19, and/or 20 and/or genes detected by probes listed in Tables 1, 3, 5, 17 and/or 18 or a subset thereof including any number between for example 1 and 121 genes.

The term “threshold” as used herein refers to a predetermined numerical value or range that corresponds to a level of gene expression or summed levels of gene expression level or range at which a subject is more likely to have a particular clinical outcome compared to a subject with a level of gene expression or summed level of gene expression below the threshold. The threshold can be selected according to a desired level of accuracy or specificity, for example the threshold can be a median level in a population, for example subjects with AML, or an average level in a population of subjects with known outcome, e.g. poor prognosis. The threshold or threshold can correspond to an average of the highest 50%, 40%, 30%, 20% or 10% expression levels in subjects with poor outcome.

The term “kit control” as used herein means a suitable assay control useful when determining an expression level of a LSC or HSC signature gene or set of genes. For kits for detecting RNA levels for example by hybridization, the kit control can comprise an oligonucleotide control, useful for example for detecting an internal control such as GAPDH for standardizing the amount of RNA in the sample and determining relative biomarker transcript levels. The kit can control can also include RNA from a cell line which can be used as a ‘baseline’ quality control in an assay, such as an array or PCR based method.

The term “hybridize” as used herein refers to the sequence-specific non-covalent binding interaction with a complementary nucleic acid. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed. With respect to an array, appropriate stringency conditions can be found and have been described for commercial microarrays, such as those manufactured and/or distributed by Agilent Inc, Affymetrix Inc, Roche-Nimblegen Inc. and other entities.

The term “microarray” or “array” as used herein refers to an ordered set of probes fixed to a solid surface that permits analysis such as gene analysis of a set of genes. A DNA microarray refers to an ordered set of DNA fragments fixed to the solid surface. For example, the microarray can be a gene chip. Methods of detecting gene expression and determining gene expression levels using arrays are well known in the art. Such methods are optionally automated.

The term “isolated nucleic acid sequence” as used herein refers to a nucleic acid substantially free of cellular material or culture medium when produced by recombinant DNA techniques, or chemical precursors, or other chemicals when chemically synthesized.

The term “polynucleotide”, “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand.

The term “probe” as used herein refers to a nucleic acid molecule that comprises a sequence of nucleotides that will hybridize specifically to a target nucleic acid sequence e.g. a coding sequence of a gene listed herein including in Table 2, 4, 6, 12 and/or 14. For example the probe comprises at least 10 or more, 15 or more, 20 or more bases or nucleotides that are complementary and hybridize contiguous bases and/or nucleotides in the target nucleic acid sequence. The length of probe depends on the hybridization conditions and the sequences of the probe and nucleic acid target sequence and can for example be 10-20, 21-70, 71-100, 101-500 or more bases or nucleotides in length. For example, the probe can comprise a sequence provided herein, including those listed in any one of Tables 1, 3, 5, 17 or 18 (e.g. comprise any one of SEQ ID NO:s 1-2533). The probes can optionally be fixed to a solid support such as an array chip or a DNA microarray chip.

A person skilled in the art would recognize that “all or part of” of a particular probe or primer can be used as long as the portion is sufficient for example in the case a probe, to specifically hybridize to the intended target and in the case of a primer, sufficient to prime amplification of the intended template.

The term “probe set” as used herein refers to a set of probes that hybridize with the mRNA of a specific gene and identified by a probe set ID number, such as 209993_at, 206385_at and others as listed in Table 1, 3 5, 17 or 18. Each probe set comprises one or more probes, for example 2, 3, 4, 5, 6, 7, 8, 9, 10, 11, 12 or more probes.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides or any number in between, although it can contain less. The factors involved in determining the appropriate length of primer are readily known to one of ordinary skill in the art.

The term “antibody” as used herein is intended to include monoclonal antibodies, polyclonal antibodies, and chimeric antibodies. The antibody may be from recombinant sources and/or produced in transgenic or non-transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

To produce polyclonal antibodies, animals can be injected once or repeatedly with an antigen representing a peptide fragment of the protein product corresponding to the nucleotide sequence of interest, alone or in conjunction with other proteins, potentially in combination with adjuvants designed to increase the immune response of the animal to this antigen or antigens in general. Polyclonal antibodies can then be harvested after variable lengths of time from the animal and subsequently utilized with or without additional purification. Such techniques are well known in the art.

To produce human monoclonal antibodies, antibody producing cells (lymphocytes) can be harvested from a human having cancer and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with cancer cells and the monoclonal antibodies can be isolated.

Specific antibodies, or antibody fragments, reactive against particular target polypeptide gene product antigens (e.g. Table 2, 4, 6, or 14 polypeptide), can also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)).

As used herein “a user interface device” or “user interfaced” refers to a hardware component or system of components that allows an individual to interact with a computer e.g. input data, or other electronic information system, and includes without limitation command line interfaces and graphical user interfaces.

The term “similar” in the context of a gene expression level as used herein refers to a subject gene expression level that falls within the range of levels associated with a particular class e.g. prognosis, for example associated with a particular disease outcome, such as likelihood of survival.

The term “most similar” in the context of a reference expression profile refers to a reference expression profile that shows the greatest number of identities and/or degree of changes with the subject expression profile.

The phrase “therapy”, treatment”, or “treatment regimen” as used herein, refers to an approach aimed at obtaining beneficial or desired results, including clinical results and includes medical procedures and applications including for example chemotherapy, pharmaceutical interventions, surgery, radiotherapy, bone marrow transplant, stem cell transplant and naturopathic interventions as well as test treatments for treating hematological cancers. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” or “treatment regimen” can also mean prolonging survival as compared to expected survival if not receiving treatment.

Moreover, a “treatment” or “prevention” regime of a subject with a therapeutically effective amount of a compound of the present disclosure may consist of a single administration, or alternatively comprise a series of applications.

A “suitable treatment” as used herein refers to a treatment suitable according to the determined prognosis. For example, a suitable treatment for a subject with a poor prognosis can include a more aggressive treatment, for example, in the case of AML, this can include a bone marrow transplant.

As used herein, “screening a new drug candidate” refers to evaluating the ability of a new drug or therapeutic equivalent to target CSCs for example LSCs in a hematological cancer.

As used herein, the term “molecular risk status” refers to the presence or absence of molecular risk factors associated with prognosis. For example, a subject in a “high molecular risk (HMR) group” includes a subject having NPM1wt/FLT3wt or FLT3ITD positive CN AML which is associated with poor prognosis; and a subject in a “low molecular risk (LMR) group” includes a subject with NPM1mut/FLT3wt CN AML.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

### II. Methods and Computer Product

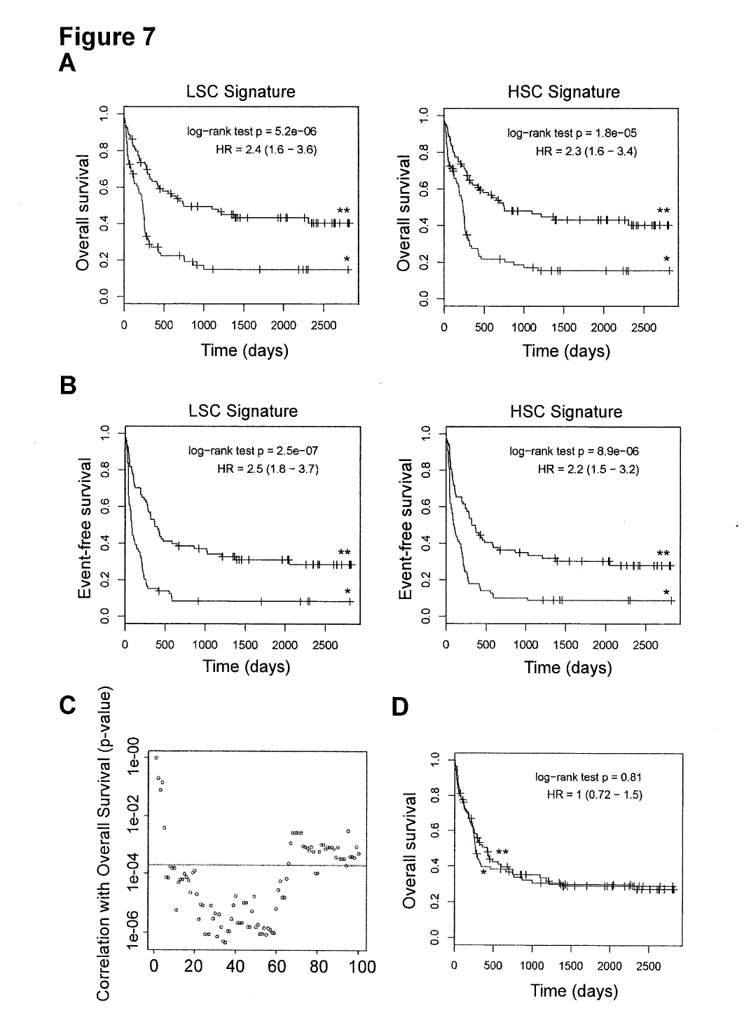

It is demonstrated herein that a LSC gene expression profile comprising for example 25 probe sets (Table 1, SEQ ID NO:1-280) corresponding to 23 genes (Table 2), 48 probe sets (Table 5; SEQ ID NO:1-280 and 759-1011) corresponding to 42 genes (Table 6) as well as smaller and larger probe sets (see FIG. 7c and Table 16) were able to distinguish patients with a poor prognosis from patients with a good prognosis. As an example, the top twenty-five probe sets associated with LSC within a FDR of 0.05 were chosen and assessed for prognostic ability as shown in Example 1. As another example, the top 48 probe sets associated with LSC within a FDR of 0.05 were chosen and assessed for prognostic ability as shown in Example 6. Other probes set groups comprising other numbers of probes sets are also predicted and herein shown to be prognostic (see for example FIG. 7c and Table 16).

It is also demonstrated herein that a HSC gene expression profile comprising 43 probe sets (Table 3; SEQ ID NO:281-758) corresponding to 39 genes (Table 4) were able to distinguish AML patients with a poor prognosis from patients with a good prognosis. It is also demonstrated herein that an HSC gene expression profile comprising 147 probesets (Table 3 and 17) and 121 genes (Table 14) could also distinguish AML patients with a poor prognosis from patients with a good prognosis. The forty-three HSC signature probesets were identified using an ANOVA test (FDR 0.01) and the 147 signature probesets were identified using an one-way ANOVA analysis using Tukey HSD post-hoc test and Benjamini-Hochberg multiple testing correction (FDR 0.05). Other gene marker sets and/or probes sets comprising other numbers of genes or probe sets are also predicted to be prognostic.

An aspect of the disclosure includes a method for determining prognosis of a subject having a hematological cancer, comprising:
a) determining a gene expression level of each of a set of genes, selected from leukemia stem cell (LSC) signature genes, a hematopoietic stem cell (HSC) signature genes and/or CE-HSC/LSC signature genes, in a sample taken from the subject;
b) correlating the gene expression levels of the set of genes with a prognosis; and
c) providing the prognosis associated with the gene expression levels.

In an embodiment, increased expression of the set of genes compared to a control (e.g. a subject or subjects with good prognosis) is indicative of a poor prognosis. In an embodiment, decreased expression compared to a control, in indicative of a good prognosis. In an embodiment, the gene expression levels is correlated with a prognosis by comparing to one or more reference profiles associated with a prognosis, wherein the prognosis associated with the reference expression profile most similar to the expression levels is the provided prognosis.

In an embodiment, the set of genes includes 2 or more genes described herein (e.g. listed in the Tables and/or detectable by a probe or probeset described herein).

An embodiment, includes a method for determining prognosis in a subject having a hematological cancer comprising:
a) determining an expression level for each gene of set a set of genes selected from leukemia stem cell (LSC) signature genes listed in Tables 2, 6 and/or 12, hematopoietic stem cell (HSC) signature genes listed in Tables 4, and/or 14, and/or CE-HSC/LSC signature genes listed in Table 19, to obtain a subject expression profile of a sample obtained from the subject; and
b) classifying the subject as having a good prognosis or a poor prognosis based on the subject expression profile;
   wherein a good prognosis predicts an increased likelihood of survival within a predetermined period after initial diagnosis and poor prognosis predicts a decreased likelihood of survival within the predetermined period after initial diagnosis.

As further described below, the subject can be classified by comparing the subject expression profile to one or more reference profiles associated with a prognosis and identifying the reference profile most similar to the subject expression profile thereby classifying the subject. In an embodiment, the subject is classifying by calculating a subject risk score and comparing the subject risk score to a threshold, wherein a subject risk score greater than the threshold classifies the subject as having a poor prognosis and a subject risk score less than the threshold classifies the subject as having a good prognosis. In an embodiment, the threshold is the median score associated with a population of subjects.

In an embodiment, the set of genes comprises at least 2 genes. As demonstrated in FIG. 17 for example, a LSC gene signature comprising 2 genes can differentiate AML subjects that have a poor survival from subjects that have a good survival is statistically significant.

Accordingly, an embodiment includes a method for determining prognosis in a subject having a hematological cancer comprising:

a) determining a gene expression level for each gene of a set of genes selected from Tables 2, 6, 12, 4, 14, 13 and/or 19 (e.g. LSC signature genes listed in Tables 2, 6, and/or 12 and/or hematopoietic stem cell (HSC) signature genes listed in Tables 4 and/or 14, and/or CE-HSC/LSC signature genes listed in Tables 13 or 19), to obtain a subject expression profile of a sample from the subject, wherein the set of genes comprises at least 2 genes; and

b) classifying the subject as having a good prognosis or a poor prognosis based on the subject expression profile;

wherein a good prognosis predicts an increased likelihood of survival within a predetermined period after initial diagnosis and poor prognosis predicts a decreased likelihood of survival within the predetermined period after initial diagnosis, compared optionally to a median outcome for the hematological cancer.

A further embodiment includes a method for determining prognosis in a subject having a hematological cancer comprising:
a) determining a gene expression level of each of a set of genes selected from LSC signature genes listed in Tables 2, 6, and/or 12, to obtain a subject expression profile in a sample from the subject, wherein the set of genes comprises at least 2 genes; and
b) classifying the subject as having a good prognosis or a poor prognosis based on the subject expression profile;
   wherein a good prognosis predicts an increased likelihood of survival within a predetermined period after initial diagnosis and poor prognosis predicts a decreased likelihood of survival within the predetermined period after initial diagnosis.

Table 12 comprises a list of the top 80 most predictive probesets and the genes detected by the probesets. Table 2 comprises 25 probesets that detect 23 genes and Table 6 comprises 48 probesets that detect 42 genes. The genes listed in Table 2 and 6 are also found in Table 12 and the genes listed in Table 2 are also found in Table 6. In an embodiment, the set of genes is selected from Table 6. In a further embodiment, the set of genes comprises the genes listed in Table 6.

Yet another embodiment includes a method for determining prognosis in a subject having a hematological cancer comprising:
a) determining a gene expression level of each gene of a set of genes selected from HSC signature genes listed in Tables 4 and/or 14, to obtain a subject expression profile in a sample from the subject, wherein the set of genes comprises at least 2 genes; and
b) classifying the subject as having a good prognosis or a poor prognosis based on the expression profile;
   wherein a good prognosis predicts an increased likelihood of survival within a predetermined period after initial diagnosis and poor prognosis predicts a decreased likelihood of survival within the predetermined period after initial diagnosis.

Table 4 comprises 48 probesets, which detect 39 genes and Table 14 comprises 149 probesets that detect 121 genes. Table 20 includes a subset of HSC signature genes that were analyzed by qRT-PCR analysis. The genes listed in Table 20 are also found in Table 14. In an embodiment, the set of genes is selected from Table 20.

A further embodiment, includes a method for determining prognosis in a subject having a hematological cancer comprising:
a) determining a gene expression level of each gene of a set of genes selected from CE-HSC/LSC signature genes listed in Table 19, to obtain a subject expression profile in a sample from the subject, wherein the set of genes comprises at least 2 genes; and
b) classifying the subject as having a good prognosis or a poor prognosis based on the expression profile;
   wherein a good prognosis predicts an increased likelihood of survival within a predetermined period after initial diagnosis and poor prognosis predicts a decreased likelihood of survival within the predetermined period after initial diagnosis.

Table 19 comprises a subset of HSC signature genes that are also expressed in LSC. Table 13 comprises a subset of the Table 19 genes. In an embodiment, the set of genes is selected from Table 13.

As mentioned, signatures comprising 2 genes can differentiate AML patients with poor and good survival. In an embodiment, at least one of the set of genes is ceroid-lipofuscinosis, neuronal 5 (CLN5) or neurofibromin 1 (NF1) In an embodiment, CLN5 is detected by one or mores of probe set ID: 214252_s_at. In an embodiment, NF1 is detected by one or more probes of probe set ID 212676_at.

Two genes overlap (RBPMS and FRMD4B) between the HSC and LSC signatures, or between the LSC and CE-HSC/LSC lists. In an embodiment, the set of genes comprises RBPMS and/or FRMD4B.

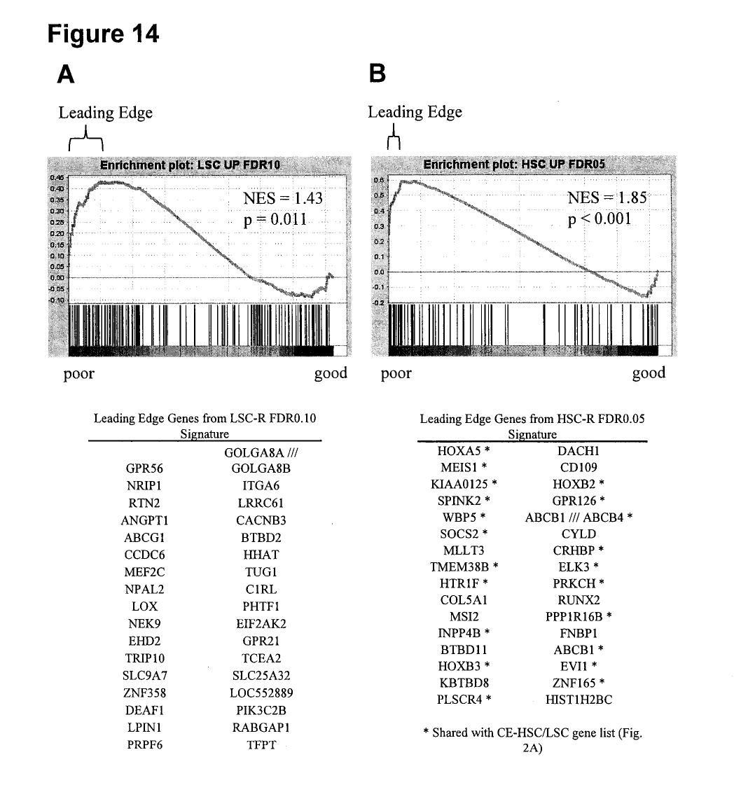

FIGS. 14a and 14b, shown an analysis of enrichment of LSC (14A) or HSC (14B) signatures in the expression data for poor cytogenetic risk AML vs good cytogenetic risk AML. FIGS. 14a and 14b show that the stem cell signatures correlate with the gene expression in poor risk AML vs good risk. In an embodiment, the set of genes comprises 2 or more of the genes listed in FIG. 14a and/or FIG. 14b.

FIG. 14 also lists ‘leading edge’ genes. In an embodiment, the set of genes comprises 2 or more of the leading edge genes in FIG. 14a and/or 14b. Also of the HSC leading edge genes, 21 overlap with the 44 CE-HSC/LSC signature gene list. Accordingly in an embodiment, the set of genes comprises 2 or more of the 21 overlap genes. In an embodiment, the set comprises at least 5, at least 10, at least 15, at least 20 or 21 of the 21 overlap genes.

Determination of prognosis, e.g. good prognosis or poor prognosis, involves in an embodiment, classifying a subject with a hematological cancer such as leukemia, based on the similarity of a subject's gene expression profile to a reference expression profile associated with a particular outcome. Accordingly, in an embodiment, the disclosure provides a method for classifying a subject having a hematological cancer as having a good prognosis or a poor prognosis, comprising:
a) calculating a first measure of similarity between a subject expression profile and a good prognosis reference profile and a second measure of similarity between the subject expression profile and a poor prognosis reference profile; the subject expression profile comprising the expression levels of a first set of genes in a sample from the subject; the good prognosis reference profile comprising, for each gene in the first set of genes, the average expression level of the gene in a set of good prognosis subjects; and the poor prognosis reference profile comprising, for each gene in the first set of genes, the average expression level of the gene in a set of poor prognosis subjects, the first set of genes comprising at least 2, or at least 5 of the genes listed in Table 2, 4 6, 12, 13, 14, 19, and/or 20 and/or genes detected by probes listed in Tables 1, 3, 5, 17 and/or 18;
b) classifying the subject as good prognosis if the subject expression profile has a higher similarity to the good prognosis reference profile than to the poor prognosis reference profile, or classifying the subject as poor prognosis if the subject expression profile has a higher similarity to the poor prognosis reference profile than to the good prognosis reference profile.

A number of algorithms can be used to assess similarity. For example, a Naïve Bayes probabilistic model is trained on data. In order to stratify the class of a new patient (prognosis of survival/non-survival) the Naïve Bayes classifier combines this probabilistic model with a decision rule: assign the sample to the class (survival/non-survival)) that is most probable; this is known as the maximum a posteriori or MAP decision rule.

The similarity can also be assessed by determining if the similarity between a subject expression profile and a reference profile is above or below a predetermined threshold. For example, the expression profile can be summed to provide a subject risk score. If the score is above a selected or predetermined threshold, the subject has a poor prognosis and if below the threshold the subject has a good prognosis.

In an embodiment, the subject expression profile is used to calculate a subject risk score, wherein the subject is classified as having a good prognosis if the subject risk score is low and as having a poor prognosis if the subject risk score is high. For example, the gene expression of 5 or more genes of a LSC and/or HSC signature genes could be determined by microarray analysis wherein the microarray comprises probes and/or probe sets directed to for example the 5 or more of the LSC and/or HSC signature genes The microarray results could be scaled to a standard expression range, (e.g. for example as determined using the 160 AML patients described in the Examples). An expression score is calculated from the summed expression levels detected using the probe or probe sets (e.g. one or more of the probes or probe sets listed in Tables 1, 3, 5, 17 and/or 18, or one or more probe sets selected from SEQ ID NOs:1-2533 and compared to a reference score or threshold (e.g. such as the median expression score of the 160 AML samples form the initial dataset) to determine if the subject falls within the poor prognosis or the good prognosis category based on the expression profile. In an embodiment, an expression profile is used to calculate a subject risk score, wherein the subject is classified as having a good prognosis if the subject risk score is below for example, a median risk score or threshold and as having a poor prognosis if for example the subject risk score is above the median or threshold. In another embodiment, an expression score or subject risk score is calculated by: a) calculating the log 2 expression value of the LSC or HSC gene signature marker set for the sample; b) centering the log 2 expression value of step b) to a zero mean; c) taking the sum of the log 2 expression values.

The predetermined period can vary depending on the likelihood of a particular outcome. In another embodiment, the predetermined period is 1 year, 2 years, 3 years, 4 years or 5 years.

The reference profiles and thresholds can be pre-generated, for example the reference expression profiles can be comprised in a database or generated de novo.

In an embodiment, the methods are used to measure treatment response. For example, the group used to test the prognostic power of the gene expression signature profiles described herein were therapeutically treated. The expression profiles were obtained prior to treatment and outcome was determined after treatment. Accordingly, the methods can be used to predict treatment response wherein a subject expression profile associated with poor prognosis is indicative of an increased likelihood of a poor or no treatment response and a subject expression profile associated with a good prognosis is indicative of an increased likelihood of a treatment response compared to for example the median response for example, the median response for the leukemia. Therefore, in an aspect, the disclosure includes a method for monitoring a response to a cancer treatment in a subject having a hematological cancer, comprising:
a. collecting a first sample from the subject before the subject has received the cancer treatment;
b. collecting a subsequent sample from the subject after the subject has received the cancer treatment;
c. determining the gene expression levels of a set of genes selected from LSC signature genes, HSC signature genes and/or CE-HSC/LSC signature genes in the first and the subsequent samples according to a method described herein, to obtain a first sample subject expression profile and a subsequent sample subject expression profile, wherein the set of genes comprises at least 2 genes; and
d. calculating a first sample subject risk score and a subsequent sample subject risk score;
   wherein a lower subsequent sample risk score compared to the first sample risk score is indicative of a positive response, and a higher subsequent sample risk score compared to the first risk score is indicative of a negative response.

In another aspect, the methods described herein are used to screen for a putative drug candidate for a hematological cancer. In an embodiment the method comprises: contacting a test population of cells with a test substance; determining a gene expression level for each gene of a set of genes selected from leukemia stem cell (LSC) signature genes listed in Tables 2, 6, and/or 12, hematopoietic stem cell (HSC) signature genes listed in Tables 4 and/or 14, and/or CE-HSC/LSC signature genes listed in Table 19, to obtain an expression profile for the test population of cells and comparing to a control population of cells; calculating an expression score for the test population of cells and the control population of cells wherein a decreased expression score in the test population of cells compared to the control population is indicative that the test substance is a putative drug candidate. In an embodiment, the test and control population of cells are hematological cancer cells.

In an embodiment, the set of genes comprises 2 or more of the genes listed in Table 2, 6, and/or 12 and the set of genes comprises 2 or more of the genes listed in Table 4 and/or 14. In another embodiment, the set of genes comprises 2 or more of the genes listed in Table 20. In another embodiment, the set of genes comprises 2 or more of the genes listed in Table 13 or Table 19.

In a further embodiment, the set of genes comprises at least at least 2-5, at least 6-10, at least 11-15, at least 16-20, at least 20-25, at least 26-30, at least 31-35, at least 36-40 or at least 41, at least 42 or at least 43, at least 41-45, at least 46-50, at least 51-55, at least 56-60, at least 61-65, at least 66-70, at least 71-75, at least 76-80, at least 81-85, at least 86-90, at least 91-95, at least 96-100, at least 101-105, at least 106 to 110, at least 111 to 115, at least 116 to 120 or 121 genes.

In an embodiment, the set of genes comprises the genes listed in Table 2, 4, 6, 12, 13, 14, 19 or 20. In an embodiment, the set of genes comprises the genes listed in Table 19. In another embodiment, the set of genes comprises the genes listed in Table 13.

In an embodiment, the set of genes does not include one or more of ABCB1, BAALC, ERG, MEIS1, and EVI1 (also known as MECOM).

In another embodiment, the gene expression levels are determined using probes and/or probe sets. In an embodiment, the probes and probe sets are selected from SEQ ID NOs: 1 to 2533.

In an embodiment, the gene expression levels are determined using at least 2-5, at least 6-10, at least 11-14, at least 15-19, at least 20-24, or 25 LSC probe sets listed in Table 1; and/or at least 2-5, at least 6-10, at least 11-15, at least 16-20, at least 21-25, at least 26-30, at least 31-35, at least 36-40, at least 41-45 at least 46-50, at least 51-55, at least 56-60, at least 61-65, at least 66-70, at least 71-75, least 81-85, at least 86-90, at least 91-95, at least 96-100, at least 101-105, at least 106-110, at least 111-115, at least 116-120, at least 121-125, at least 126-130, at least 131-135, at least 136-140, at least 141-145, or at least 146-147 probe sets. In an embodiment, combinations of probes and probes sets listed in different tables are used to determine the gene expression levels.

Successive addition of the most highly ranked, determined by p-value, probes demonstrated a correlation with overall survival (FIG. 7c). For example, successive addition of the top 35 probes, showed the greatest correlation with overall survival. Therefore, in still another embodiment, the gene expression level is determined by one or more probes and/or one or more probe sets selected from probesets listed in Table 16.

In yet another embodiment, a method described herein also comprises obtaining a sample from the subject, e.g. for determining the expression level of the set of genes. The sample, in an embodiment, comprises a blood sample or a bone marrow sample. In an embodiment, the sample comprises fresh tissue, frozen tissue sample, a cell sample, or a formalin-fixed paraffin-embedded sample. In an embodiment, the sample is submerged in a RNA preservation solution, for example to allow for storage. In an embodiment, the sample is submerged in Trizol®. In an embodiment, the sample is stored as soon as possible at ultralow (for example, below −190° C.) temperatures. Storage conditions are designed to maximally retain mRNA integrity and preserve the original relative abundance of mRNA species, as determined by those skilled in the art. The sample in an embodiment is optionally processed, for example, to obtain an isolated RNA fraction and/or an isolated polypeptide fraction. The sample is in an embodiment, treated with a RNAse inhibitor to prevent RNA degradation.

In another embodiment, the sample is a fractionated blood sample or a fractionated bone marrow sample. In an embodiment, the sample is fractionated to increase the percentage of LSC and/or HSC. In an embodiment, the fraction is predominantly for example greater than 90% CD34+. In another embodiment, the fraction is predominantly, for example greater than 90% CD38−. In a further embodiment, the fraction is predominantly, for example greater than 90% CD34+ and CD38−.

Wherein the gene expression level being determined is a nucleic acid, the gene expression levels can be determined using a number of methods for example a microarray chip or PCR, optionally multiplex PCR, northern blotting, or other methods and techniques designed to produce quantitative or relative data for the levels of mRNA species corresponding to specified nucleotide sequences present in a sample. These methods are known in the art. In an embodiment, the gene expression level is determined using a microarray chip and/or PCR, optionally multiplex PCR.

Further, for example a person skilled in the art would be familiar with the necessary normalizations necessary for each technique.

The methods described can utilize probes or probe sets comprising or optionally consisting of a nucleic acid sequence listed in Tables 1, 3, 5, 17 and/or 18. In an embodiment, the gene expression level is determined by detecting mRNA expression using one or more probes and/or one or more probe sets listed in Tables 1, 3, 5, 17 and/or 18.

In an embodiment, the method comprises additionally considering known prognostic factors, such as molecular risk status. For example, the mutational status of FLT3ITD and NPM1 has been associated with risk status in AML subjects, with low molecular risk associated with NPM1mut FLT3ITD− and high molecular risk associated with FLT3ITD+ or NPM1wtFLT3ITD−. It is demonstrated herein that the gene signatures can further stratify for example molecular risk subjects to identify subjects with poor prognosis.

Accordingly, in an embodiment, the method further comprises determining the molecular risk status of the subject. In an embodiment, the molecular risk status is low molecular risk (LMR) or high molecular risk (HMR) according to NPM1 and/or FLT3ITD status, wherein the subject is identified as LMR if the subject comprises a mutant NPMI gene and is FLT3IT positive, and is identified as HMR if the subject has a wildtype NPMI gene and is FLT3ITD negative. In a further embodiment, the subject is LMR and optionally the set of genes comprises genes selected from LSC signature genes. In an embodiment, the subject is HMR and optionally the set of comprises genes selected from HSC signature genes.

In an embodiment, the methods described herein can be used for example to select subjects for a clinical trial.

In an embodiment, the methods described herein can be used to select suitable treatment. For example, subjects with poor prognosis e.g. a high risk of non-survival may be advantageously treated with specific therapeutic regimens. More accurate classification can reduce the number of patients identified as high risk. Further, more accurate classification allows for treatments to be tailored and for aggressive therapies with greater risks or side effects to be reserved for patients with poor outcome. For example, CN-AML patients are considered intermediate risk of poor prognosis. One therapeutic option for treating AML is transplant. Given the intermediate risk, one option available to a patient is transplant, particularly if there was a related donor. However, where only an unrelated donor is available, because of complications, a transplant may not be recommended or carry additional risks. An application of the methods and products described herein is to provide a test to aid a medical professional in making such a decision. For example, where a patient has an intermediate risk but is identified by the methods and products described herein as having an increased likelihood of a good outcome, such a patient may be reclassified in a more “favorable’ category such that a transplant might not be recommended. Similarly, if the methods and products identified the patient as having an increased likelihood of a poor prognosis, the patient may be reclassified in a more “unfavorable’ category suggesting that a transplant, even from unrelated donors might be indicated. Accordingly, a better prognostic prediction could assist in making treatment decisions.

Accordingly in another aspect, the disclosure includes a method further comprising the step of providing a cancer treatment to a subject consistent with the disease outcome prognosis. In an embodiment, the disclosure provides use of a prognosis determined according to the method described herein, and identifying a suitable treatment for treating a subject with a hematological cancer. An embodiment includes a method of treating a subject having a hematological cancer, comprising determining a prognosis of the subject according to a method described herein and providing a suitable cancer treatment to the subject in need thereof according to the prognosis determined.

In another embodiment, the method further comprises providing a cancer treatment for the subject consistent with the molecular risk group and disease outcome prognosis. In an embodiment the cancer treatment is a stem cell transplant.

In an embodiment, the cancer treatment comprises a stem cell transplant. In another embodiment, the cancer treatment comprises a bone marrow transplant, or other standard treatment, such as chemotherapy.

In addition to being able to differentiate AML patients according to prognosis, the HSC signature is expected to be able to differentiate patients with hematological cancers other than AML, particularly other leukemias, that like AML for example have an altered growth and differentiation block and/or hematological cancers that are CSC hematological cancers. For example, it is myeloid leukemias such as MDS (Myelodysplastic Syndrome) or MPD (myeloproliferative disease, including CML—chronic myeloid leukemia which is considered a stem cell disease.

In an embodiment, the hematological cancer is leukemia. In an embodiment, the leukemia is acute myeloid leukemia (AML). In an embodiment, the hematological cancer is cytogenetically normal. In another embodiment, the AML is cytogenetically normal AML (CN-AML). In a further embodiment, the AML is M1, M2, M4, M4eO, M5, M5a, M5b, or unclassified AML. In yet a further embodiment, the AML is MO, M6, M7 or M8 AML. In another embodiment, the leukemia is ALL, CLL or CML or a subtype thereof. In another embodiment, the hematological cancer is lymphoma. In a further embodiment, the hematological cancer is multiple myeloma.

The methods described herein can be implemented using a computer.

Another aspect of the disclosure includes a computer-implemented method for determining a prognosis of a subject having a hematological cancer comprising: classifying, on a computer, the subject as having a good prognosis or a poor prognosis based on a subject expression profile comprising measurements of expression levels of a set of genes in a sample from the subject, the set of genes selected from genes listed in Table 2, 4 6, 12, 13, 14, 19, and/or 20 and/or genes detected by probes listed in Tables 1, 3, 5, 17 and/or 18; wherein a good prognosis predicts increased likelihood of survival within a predetermined period after initial diagnosis, and wherein a poor prognosis predicts a decreased likelihood of survival within the predetermined period after initial diagnosis.

In an aspect, the disclosure provides a computer-implemented method for determining a prognosis of a subject having a hematological cancer comprising: classifying, on a computer, the subject as having a good prognosis or a poor prognosis based on an expression profile comprising measurements of expression levels of a set of genes selected from LSC signature genes or HSC signature genes in a sample from the subject; wherein a good prognosis predicts an increased likelihood of survival within a predetermined period after initial diagnosis, and wherein a poor prognosis predicts a decreased likelihood of survival within the predetermined period after initial diagnosis. In an embodiment, the set of genes comprises at least one gene of the LSC signature genes or the HSC signature genes.

The results or the results of a step are optionally displayed or outputted. Accordingly, in an embodiment, the method further comprises displaying or outputting a result of one of the steps to a user interface device, a computer readable storage medium, a monitor, or a computer that is part of a network.

Another aspect of the disclosure includes a computer product for implementing the methods described herein e.g. for predicting prognosis, selecting patients for a clinical trial, or selecting therapy.

A further aspect of the disclosure provides a non-transitory computer readable storage medium with an executable program stored thereon, wherein the program is for predicting outcome or prognosis in a subject having a hematological cancer, and wherein the program instructs a microprocessor to perform one or more of the steps of any of the methods described herein.

A computer system comprising:
a) a user interface capable of receiving and/or inputting a selection of subject gene expression levels of a set of genes, the set comprising at least 2 genes listed in Table 2, 4 6, 12, 13, 14, 19, and/or 20 and/or genes detected by probes listed in Tables 1, 3, 5, 17 and/or 18, for use in comparing to the gene reference expression profiles in the database;
b) a reference database including records comprising reference expression profiles associated with clinical outcomes, each reference profile comprising the expression levels of a set of genes listed in Table 2, 4 6, 12, 13, 14, 19, and/or 20 and/or genes detected by probes listed in Tables 1, 3, 5, 17 and/or 18;
c) an analysis module for comparing the received or inputted selection of subject gene expression levels to the reference expression profiles and identifying a most similar reference profile and associated prognosis; and
d) an output that displays a prediction of prognosis according to the expression levels of the set of genes.

An exemplary system is a computer system having for example: a central processing unit; a main non-transitory storage unit, for example, a hard disk drive, for storing software and data, the storage unit controlled by storage controller; a system memory, preferably high speed random-access memory (RAM), for storing system control programs, data, and application programs, for example for viewing and manipulating data, evaluating formulae for the purpose of providing a prognosis, comprising programs and data loaded from non-transitory storage unit; system memory may also include read-only memory (ROM); a user interface, comprising one or more input devices (e.g., keyboard) and a display or other output device; a network interface card for connecting to any wired or wireless communication network (e.g., a wide area network such as the Internet); a communication bus for interconnecting the aforementioned elements of the system; and a power source to power the aforementioned elements. Operation of computer is controlled primarily by operating system, which is executed by central processing unit. Operating system can be stored in system memory. In addition to an operating system, in a typical implementation system memory includes: a file system for controlling access to the various files and data structures used by the methods and computer products disclosed herein. The system memory can optionally include a coprocessor dedicated to carrying out mathematical operations.

Another aspect includes a computerized control system 10 for carrying out the methods of the disclosure.

In an embodiment, the computerized control system 10 comprises at least one processor and memory configured to provide:
a) a control module 20 to receive a dataset comprising a subject expression profile comprising a set of gene expression levels for a set of genes, each gene of the set of genes selected from LSC signature genes listed in Tables 2, 6 and/or 12 or HSC signature genes listed in Tables 4 and/or 14;
c) an analysis module 30 to:
   i) compare the subject expression profile to a reference expression profile comprising an expression level for each gene of the set of genes; and
   ii) identify a prognosis associated with the subject expression levels.

A schematic representation of an embodiment of a computerized control system 10 is provided in FIG. 17.

In an embodiment, the set of genes is selected from Tables 2, 4 6, 12, 13, 14, 19, and/or 20 and/or genes detected by probes listed in Tables 1, 3, 5, 17 and/or 18.

In an embodiment, the subject expression profile is compared to a reference expression profile by comparing a subject risk score to a selected threshold, wherein the subject risk score is calculated by summing the subject expression profile gene expression values, optionally the log 2 expression values, of the set of genes.

In an embodiment, the dataset is generated using an array probed with a sample obtained from the subject.

In an embodiment, the computerized control system controls and/or receives data from an imaging module 50. In an embodiment, the imaging module is a microarray scanner, which optionally detects dye fluorescence. In an embodiment, the imaging module is configured to collect the images and spot intensity signals. In an embodiment, the computerized control system 10 further comprises an image data processor for processing the image data.

In an embodiment, the analysis module 30 further determines a prognosis characteristic such as a hazard ratio or risk score.

In an embodiment, the computerized control system 10 further comprises a search module 40 for searching an expression reference databases 70 to identify and retrieve reference expression profiles associated with a prognosis.

In an embodiment, the computerized control system 10 further comprises a user interface 60 operable to receive one or more selection criteria, wherein the processor is further operable to configure the analysis module 30 to include the criteria received in the user interface 60. For example, the selection criteria can comprise a selected threshold.

A further aspect comprises a non-transitory computer-readable storage medium comprising an executable program stored thereon, wherein the program instructs a processor to perform the following steps for a plurality of gene expression levels: calculate a subject risk score; and determine a prognosis according to the subject risk score.

In an embodiment, the program further instructs the processor to determine a prognosis characteristic such as a hazard ratio.

In an embodiment, the program further instructs the processor to output a prognosis and/or a prognosis characteristic such as a hazard ratio.

In an embodiment, one or more of the user interface components can be integrated with one another in embodiments such as handheld computers.

In an embodiment, the computer system comprises a computer readable storage medium described herein.

In an embodiment, the computer system is for performing a method described herein.

### III. Compositions, Arrays and Kits

An aspect provides a composition comprising a set of probes or primers for determining expression of a set of genes. In an embodiment, the composition comprises at least 2 nucleic acid molecules each comprising a polynucleotide probe sequence selected from Tables 1, 3, 5, 17 or 18 (SEQ ID NO:1-2533. In an embodiment, the composition comprises a set of nucleic acid molecules wherein the sequence of each molecule comprises a polynucleotide probe sequence selected from SEQ ID NO:1-2533.

Another aspect includes an array comprising, for each gene in a set of genes, the set of genes comprising at least 2 of the genes listed in Table 2, 4, 6, 12, 13, 14, 19 and/or 20, one or more polynucleotide probes complementary and hybridizable to a coding sequence in the gene.

In an embodiment, the composition or array comprises at least 3-22, at least 23-44, at least 45-66, at least 67-88, at least 89-110, at least 111-132, at least 133-154, at least 155-176, at least 177-198, at least 199-220, at least 221-242, at least 243-264, at least 265-286, at least 287-308, at least 309-330, at least 331-352, at least 353-374, at least 375-396, at least 397-418, at least 419-440, at least 441-462, at least 463-478 or more nucleic acid molecules each comprising a polynucleotide probe sequence selected from Tables 1, 3, 5, 17 and/or 18 (SEQ ID NOs:1-2533 In yet another embodiment, the composition comprises 2-2533, or any number there between, nucleic acid molecules comprising or consisting of a polynucleotide probe sequence listed in Tables 1, 3, 5, 17 and/or 18 (SEQ ID NOs:1-2533).

In yet another embodiment, the composition comprises at least 2 nucleic acid molecules each comprising a polynucleotide probe sequence selected from SEQ ID NO:1-280 and 759-1011.

In yet another embodiment, the composition comprises at least 2 nucleic acid molecules each comprising a polynucleotide probe sequence selected from SEQ ID NO:281-758 and 1012 to 2533.

In another embodiment, the composition or array comprises at least 3-22, at least 23-44, at least 45-66, at least 67-88, at least 89-110, at least 111-132, at least 133-154, at least 155-176, at least 177-198, at least 199-220, at least 221-242, at least 243-264, at least 265-280, at least 281-295, at least 296-310, at least 311-325, at least 326-340, at least 341-355, at least 356-380, at least 381-395, at least 396-410, at least 411-425, at least 426-440, at least 441-455, at least 456-470, at least 471-485, at least 486-500, at least 501-515, at least 516-532 or up to 533 nucleic acid molecules/probes. In an embodiment, the composition or array comprises any number of nucleic acid molecules/probes from 3 to 2533, or more.

In another embodiment, the composition comprises at least 2 nucleic acid molecules each comprising a polynucleotide sequence selected from the probes comprised in the probe set IDs listed in Table 16.

In an embodiment, the set of genes comprises at least 3-5, at least 6-10, at least 11-15, at least 16-20, at least 21-25 of the genes listed in Table 2 and/or at least 6-10, at least 11-15, at least 16-20, at least 21-25, at least 26-30, at least 31-35, or at least 36-39 of the genes listed in Table 4, at least 6-10, at least 11-15, at least 16-20, at least 21-25, at least 26-30, at least 31-35, or at least 36-39 or at least 41-43 of the genes listed in Table 6, at least at least 6-10, at least 11-15, at least 16-20, at least 21-25, at least 26-30, at least 31-35, at least 36-39, at least 41-45, 46-66, at least 67-80, of the genes listed in Table 12 and/or at least 6-10, at least 11-15, at least 16-20, at least 21-25, at least 26-30, at least 31-35, or at least 36-39, at least 41-45, 46-66, at least 67-88, at least 89-110, or at least 111-121 of the genes listed in Table 14.

The array can be a microarray designed for evaluation of the relative levels of mRNA species in a sample.

Another aspect of the disclosure provides a kit for determining prognosis in a subject having a hematological cancer comprising:
a) an array described herein;
b) a kit control; and
c) optionally instructions for use.

A further aspect of the disclosure includes a kit for determining prognosis in a subject having a hematological cancer comprising:
a) a set of probes wherein each probe of the set hybridizes and/or is complementary to a nucleic acid sequence corresponding to at least 2, or at least 5, genes selected from Table 2, 4, 6, 12 and/or 14;
b) a kit control; and
c) optionally instructions for use.

In an embodiment, the kit further comprises one or more specimen collectors and/or RNA preservation solution.

In an embodiment, the specimen collector comprises a sterile vial or tube suitable for receiving a biopsy or other sample. In an embodiment, the specimen collector comprises RNA preservation solution. In another embodiment, RNA preservation solution is added subsequent to the reception of sample. In another embodiment, the sample is frozen at ultralow (for example, below 190° C.) temperatures as soon as possible after collection.

In an embodiment the RNA preservation solution comprises one or more inhibitors of RNAse. In another embodiment, the RNA preservation solution comprises Trizol® or other reagents designed to improve stability of RNA.

In an embodiment, the kit comprises at least 3-22, at least 23-44, at least 45-66, at least 67-88, at least 89-110, at least 111-132, at least 133-154, at least 155-176, at least 177-198, at least 199-220, at least 221-242, at least 243-264, at least 265-286, at least 287-308, at least 309-330, at least 331-352, at least 353-374, at least 375-396, at least 397-418, at least 419-440, at least 441-462 or at least 463-473 and for example up to 2533 or any number between 1 and 2533, nucleic acid molecules, each comprising and/or corresponding to a polynucleotide probe sequence listed in Table 1, 3, 5, 17 and/or 18 (SEQ ID NO:1-2533.

Another aspect of the disclosure provides a kit determining prognosis in a subject having a hematological cancer comprising:
a set of antibodies comprising at least two antibodies, wherein each antibody of the set is specific for a polypeptide corresponding to a gene selected from Table 2, 4, 6, 12 and/or 14; and
instructions for use.

In an embodiment, the kit comprises a set of antibodies specific for polypeptides corresponding to at least 2, 3, 4, 5, 6, 7, 8, 9 or at least 10 of the genes listed in Table 2, 4, 6, 12 and/or 14. In another embodiment, the kit comprises a set of antibodies specific for polypeptides corresponding to at least 11-15, 16-20, 21-25, 26-30, 31-35, 36-40, 41-45 or more of the genes listed in Tables 2, 4, 6, 12 and/or 14.

In an embodiment, the antibody or probe is labeled. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. A person skilled in the art will appreciate that a number of methods can be used to determine the amount of a polypeptide product of a gene described herein, including immunoassays such as flow cytometry, Western blots, ELISA, and immunoprecipitation followed by SDS-PAGE, as well as immunocytochemistry or immunohistochemistry. For example, flow cytometry or other methods for detecting polypeptides, can be used for detecting surface protein expression levels.

The kit can comprise in an embodiment, one or more probes or one or more antibodies specific for a gene. In another embodiment, the set or probes or antibodies comprise probes or antibodies wherein each probe or antibody detects a different gene listed in Table 2, 4, 6, 12 or 14.

In an embodiment, the kit is used for a method described herein.

The following non-limiting examples are illustrative of the present disclosure:

### EXAMPLES

### Example 1

### Methods

### Sorting of Patient AML Samples

Peripheral blood cells were collected from patients with newly diagnosed AML after obtaining informed consent according to procedures approved by the Research Ethics Board of the University Health Network. Individuals were diagnosed according to the standards of the French-American-British (FAB) classification. Cells from sixteen different samples representing 7 AML subtypes were investigated in the studies. Specifically, low density peripheral blood cells were collected from 16 AML patients representing 7 FAB subtypes (2 M1, 1 M2, 1 M4, 1 M4e, 1 M5, 4 M5a, 1 M5b, 5 unclassified) by density centrifugation over a Ficoll® gradient. Low-density mononuclear cells isolated from individuals with AML were frozen viably in FCS plus 10% (vol/vol) DMSO. For sorting of AML sub-populations, AML blasts were stained with anti-CD34-APC (Becton-Dickinson) and anti-CD38-PE (Becton-Dickinson) and were sorted using either a Dako Mo-Flo (Becton-Dickinson) cell sorter or a BD FACSAria (Becton-Dickinson). Purity of each subpopulation exceeded 95%. Fractionated cells were captured in 100% FCS and recovered by centrifugation. As a result, each AML patient sample was sorted into 4 subpopulations based upon CD34 and CD38 antibody staining and cells recovered for functional and gene expression analysis.

Transplantation of Sorted AML Cells into NOD/SCID Mice

NOD/SCID mice (Jackson Laboratory, Bar Harbor, Me.) were bred and maintained in microisolater cages. Twenty-four hours before transplantation, mice were irradiated with 2.75 to 3.45 Gy gamma irradiation from a 137Cs source. Sorted AML cells were counted and resuspended into 1-5% FCS in 1× phosphate buffered saline (PBS) pH 7.4 and injected directly into the right femur of each experimental animal. Six and a half to fifteen weeks post-transplant, mice were euthanized by cervical dislocation and hind leg bones removed and flushed with media to recover engrafted cells. Percent human AML engraftment was assessed by flow cytometry for human CD45+ staining cells (Lapidot et al., 1994).

mRNA Expression Array

mRNA was extracted using the Trizol® RNA preparation as recommended by the manufacturer (Invitrogen) and the RNA was amplified by Nugen amplification per manufacturer's instructions (NuGEN Technologies, Inc.). Probes were labeled and Affymetrix U133A (high-throughput) microarrays were run as per manufacturer's instructions. Signal was normalized by RMA followed by log 2-transformation. The LSC/primitive cell-related gene list was computed standard two-group differential expression comparison (Smyth's moderated t-test¹⁸, SCID Leukemia-Initiating Cells (SL-IC) fractions vs non-SL-IC fractions). Each probe set consists of, generally, eleven oligonucleotide probes complimentary to a corresponding gene sequence. These eleven probes are used together to measure the mRNA transcript levels of a gene sequence. Quality control measures were taken. For example, a sample was rejected as the array results obtained after measurement by Affymetrix standard techniques and prior to normalization was an outlier when compared to the other samples on a box-whisker plot.

Correlation with Overall Survival.

To assess the prognostic impact of the LSC/primitive cell related profile, the 25 probe sets that were most positively correlated with the SL-IC AML populations versus non-SL-IC populations were selected as the 25 LSC probe set signature (genes listed in Table 2; probes listed in Table 1). Publicly available overall survival and expression data was analyzed¹⁷. In short, the expression value of each probe was scaled to 0 for each probe across the 160 AML using the median value. For each AML, the expression values of the LSC probe set signature was summed for each of the 160 bone marrow AML samples. This summed value was used to divide the AML group into two equal sized populations of 80 AML each based upon above or below median expression of the summed value of the 25 LSC probe set signature. The overall survival of the two groups was examined using a Kaplan-Meier plot and log-rank (Mantel-Cox) test. Similarly, the correlation with survival and the 43 HSC probe set signature was determined in a similar way (genes listed in Table 4, probes listed in Table 3), except the 43 HSC probe sets were used instead of the 25 LSC probe sets.

### Discussion

The gene expression profile of sorted populations of AML cells enriched for SL-IC cells, the LSC cells detected in the xenotransplant assay, were analyzed and compared to those populations without SL-IC, and a LSC/primitive cell related profile (25 LSC probe set signature) was developed. When this profile was used to examine overall survival in a group of 160 AML patients, there was a significant correlation with poor overall survival. Similarly, there was an excellent correlation between a 43 HSC probe set signature and poor overall survival, even though there is only one overlapping probe set between the two independently generated stem cell/primitive cell-related lists. Additionally, the AML cells used in the generation of the 25 LSC probe set signature were peripheral blood samples and the 43 HSC probe set signature was derived from cord blood, while the 160 AML samples were bone marrow samples. This suggests that these two stem cell related profiles are robust and unique.

Other groups have developed prognostic signatures for CN-AML from gene expression data of bulk AML. This approach is unique in that it involves the generation of the gene set that is based upon SL-IC in sorted cells, a functional readout that is independent of patient outcome. Likewise, the HSC profile is based upon the SCID repopulating cell assay, not overall survival. However, these independent investigations into stem cell regulation have a similar correlation with patient outcome, indicating that a stem cell profile is relevant to leukemia, whether it is the 43 HSC probe set signature or the 25 LSC probe set signature.

### Example 2

The LSC signature and HSC signatures can be tested in additional leukemia patient sample sets, including sets of patient samples that contain cytogenetically abnormal AML, in order to further support the prognostic value of the signatures. For example, other blood cancers such as acute lymphoblastic leukemia, lymphomas, CML, and CLL can be tested.

### Example 3

The expression levels of subsets of the LSC signature genes and HSC signature genes, combinations of the genes in the LSC probe set signature and HSC probe set signature as well as shared genes such as the CE-HSC/LSC signature genes will be determined and assessed to identify and/or confirm the prognostic abilities of said gene sets according to the methods described in Example 1.

### Example 5

Similar to Example 1, using the sorting of patient AML samples, transplantation of sorted AML cells into NOD/SCID mice, mRNA expression array, and correlation with overall survival procedures a 43 gene signature marker set prognostic of outcome was identified (Table 6). The expression levels of the genes in the LSC gene signature were detected using 48 probe sets (Table 5). The 48 probe set LSC/primitive cell-related gene list was computed USING standard two-group differential expression comparison (Smyth's moderated t-test 18, SL-IC fractions vs non-SL-IC fractions). Benjamini and Hochberg multiple testing correction was performed to generate a list of 48 probe sets with a false discovery rate of 0.05.”

### Example 6

Evidence from experimental xenografts show that some solid tumours and leukemias are organized as cellular hierarchies sustained by cancer stem cells (CSC). Despite the promise of the CSC model, the relevance to human disease remains uncertain and improvements to prognosis and therapy have yet to be derived from CSC properties. Moreover, there are conflicting reports of whether tumours continue to adhere to a CSC model when enhanced xenograft assays are applied. Here it is demonstrated that 16 primary human acute myeloid leukemia (AML) samples, fractionated into 4 populations and subjected to sensitive in vivo leukemia stem cell (LSC) analysis, follow a CSC model of organization. Each fraction was subjected to gene expression analysis and a global LSC-specific signature was determined from functionally defined LSC. Similarly, using human cord blood, a hematopoietic stem cell (HSC) enriched gene signature was established. Bioinformatic analysis identified a core transcriptional program that LSC and HSC share, revealing the molecular machinery that underlies stemness properties. Both LSC and HSC signatures, when assessed against a large group of cytogenetically normal AML samples, showed prognostic significance independent of other factors. The data establishes that determinants of stemness influence clinical outcome of AML and more broadly they provide direct evidence for the clinical relevance of CSC.

The cancer stem cell (CSC) model posits that many cancers are organized hierarchically and sustained by a subpopulation of CSC at the apex that possess self renewal capacity¹. This model has elicited considerable interest within the greater cancer community especially as data is accumulating showing the relative resistance of CSC to therapy²⁻⁷. A key implication of the model is that cure should be dependent upon eradication of CSC, consequently patient outcome is determined by CSC properties. The CSC paradigm is well supported by two lines of evidence derived from xenotransplant models: primary cancer cells capable of generating a tumour in vivo can be purified and distinguished from those cancer cells that lack this ability; and CSC can be serially transplanted providing evidence for self renewer. However, there is little progress in translating understanding of CSC biology to improved prognosis or treatment of human disease. Thus, the importance of CSC outside of xenotransplant models is unclear and their relevance to human disease is not firmly established.

The best evidence to substantiate the clinical significance of CSC would be robust demonstration of improved survival in patients treated with new CSC-targeted therapeutics. In the absence of treatment data, the prognostic relevance of CSC can be indirectly established by correlating patient survival outcomes with CSC-specific biological properties determined using state-of-the-art xenograft models. By extension, the CSC hypothesis predicts that the heterogeneous survival outcomes observed within uniformly treated patient cohorts may be reflective of variation in CSC properties among patients. Emerging evidence from leukemia samples lends support to this prediction as correlative studies have associated characteristics linked to stem cell properties with outcome, such as the ability to engraft mice or surface expression of LSC-linked markers^{8, 9}. However, these studies are based upon an older xenograft model and only investigated single cohorts, nevertheless they establish the feasibility of this approach.

If CSC properties are relevant to human disease, it follows that the molecular machinery that governs the stem cell state must influence clinical outcome. However, little is currently known of the identity of the molecular regulators that govern CSC-specific properties. Experimental data shows that LSC possess stem cell functions common to all stem cells, including self renewal and the ability to produce differentiated, non-stem cell progeny¹. Murine models have been successfully used to identify a small number of genes that regulate LSC function, including MEIS1 and BMI1^{10, 11}. Gene expression profiling provides an approach to define CSC-specific attributes on a genome-wide basis. Recently, a human breast CSC signature was generated from an expression analysis where CSC-enriched populations were obtained from xenografts and some pleural effusions and compared to normal mammary cells¹². The expression of the breast CSC genes correlated with patient outcome for breast and other cancer types, although some have questioned to what degree this correlation derives from cancer-specific versus CSC-specific properties¹²⁻¹⁴. Clearly, more focused studies of global gene expression in well defined CSC and non-CSC populations from primary samples are needed to generate CSC specific signatures. Such studies should reveal the identity of important stem cell regulators and provide the basis to determine whether CSC-specific signatures correlate to clinical aspects of human disease.

The prospective isolation and subsequent functional and molecular analysis of CSC from a heterogeneous tumour population is often dependent on the distinctive expression of surface marker proteins. Historically, xenografts into SCID or NOD/SCID mice were used to confirm these early marker-dependant sorting strategies^{15, 16}. However, a series of recent studies using either syngeneic murine cancer models or NOD/SCID mice with impaired residual innate immunity have cast doubt upon the reliability of NOD/SCID mice to accurately capture all cancer stem cell activityl¹⁷⁻²⁰. For example, while previous studies observed that LSC can be prospectively isolated only from the CD34+/CD38− cell fraction of acute myeloid leukemia (AML), identical to normal HSC, an improved xenotransplant system has enabled the detection of LSC in previously non-tumourigenic populations^{15, 16, 18, 19}. In a separate example, the use of optimized xenotransplant methods radically altered the apparent detectable frequency of CSC from 1 in 10⁵ tumour cells to 1 in 4 tumour cells, a result that stands in stark contrast to other studies²⁰⁻²². These studies suggest that some human cancers may not follow the CSC model and strongly demonstrate the requirement for a sensitive xenotransplant model to confirm or refute the existence of a CSC hierarchy in each human cancer. More importantly, sample to sample variation between cell surface marker expression and CSC activity establishes an important principle, that all experiments designed to investigate CSC properties in purified cell fractions must assess, at the same time, all cell fractions with well validated tumour- or leukemia-initiation assays (e.g. in regards to determining a LSC or HSC signature.

Here 16 AML and 3 cord blood primary samples were fractionated and a sensitive xenotransplant assay was utilized to detect and functionally quantify each fraction for cells with LSC or HSC activity, respectively. Leukemia stem cell (LSC) and hematopoietic stem cell (HSC) gene expression signatures were identified based on this functional stem cell characterization of each purified cell fraction and bioinformatic analyses showed that they are closely correlated. Both signatures predict poor overall survival independently of other prognostic factors in patients with cytogenetically normal AML, demonstrating that stem cell gene expression programs determine patient outcome. Overall, the results establish the clinical relevance of LSC defined solely on the basis of functional xenotransplant assays.

### Methods

### Collection of Patient Samples and Normal Hematopoietic Cells

Peripheral blood samples were collected from patients with AML after obtaining informed consent according to the procedures approved by the Research Ethics Board of the University Health Network. Low-density mononuclear cells isolated from individuals with AML were frozen viably in FCS plus 10% vol/vol DMSO. Human cord blood cells obtained from full-term deliveries from consenting healthy donors according to the procedures approved by the Research Ethics Board of the University Health Network were processed as described³³.

### Cell Staining, Sorting and Flow Cytometry

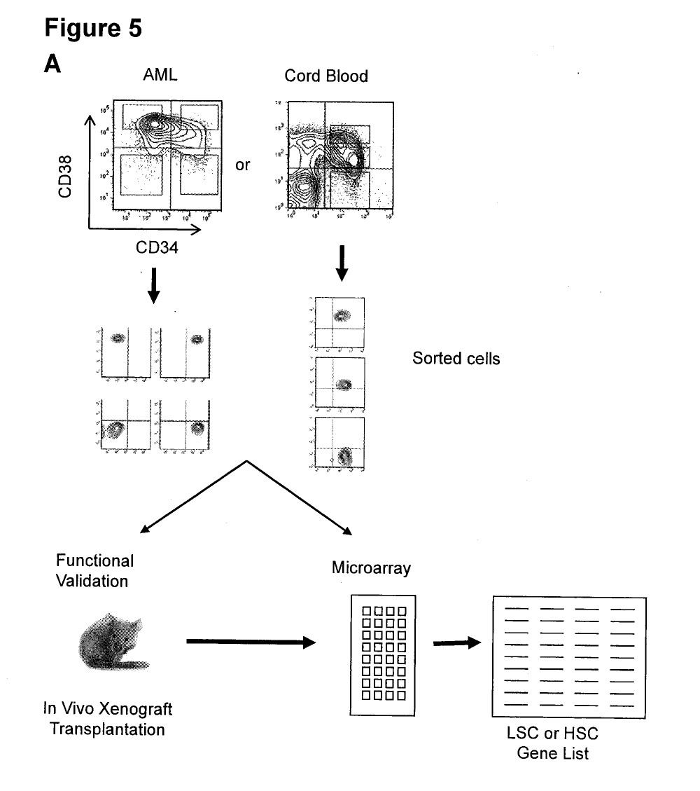

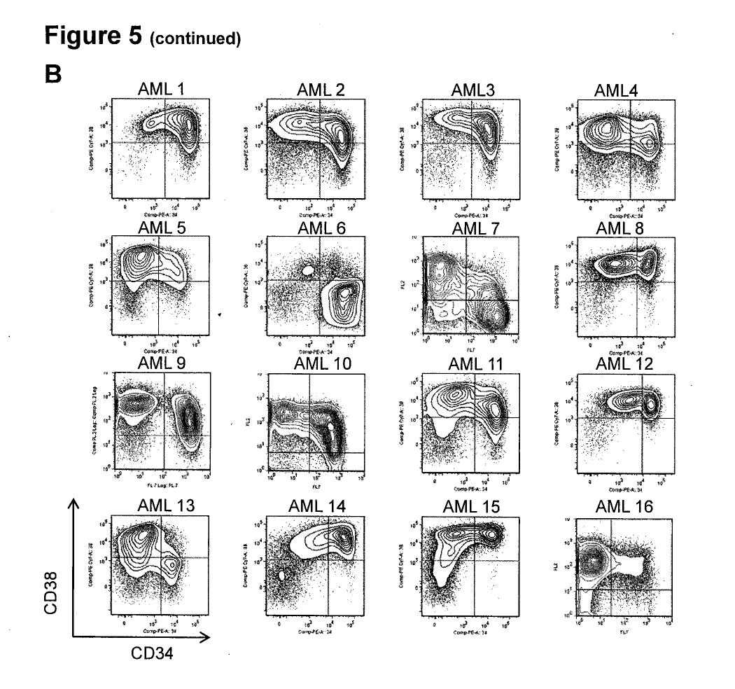

Cells were stained with antibodies to CD34, CD38, and in the case of cord blood CD36, and sorted on either a MoFlo (Beckman Coulter) or FACSAria (BD Biosciences) cells sorter. AML cells were sorted into CD34+/CD38−, CD34+/CD38+, CD34−/CD38+, CD34−/CD38− populations. Three independent pooled CB samples from 15-22 donors were used for isolation of HSC subsets and progenitors. Lin− Cord blood cells were sorted into CD34+/CD38− (HSC1), CD34+/CD38lo/CD36− (HSC2), and CD34+/CD38+ (Prog) populations. The mature cord blood fraction are cord blood cells after hemolysis (lin+). Representative sorting gates are in FIG. 5. The StemSep system (Stem Cell Technologies) was used to lineage deplete cord blood cells. Antibodies to CD34, CD38, CD15, CD14, CD19, CD33, CD45, CD36, HLA-DR, CD11b, CD117, and CD3 were used to characterize primary AML samples and AML after transplantation into mice. All antibodies were obtained from Beckman Coulter and BD Biosciences. Flow Cytometry was performed on either a FACScalibur or LSRII (BD-Biosciences).

Transplantation of Cells into NOD/scid Mice and Colony Formation Assays

NOD/ShiLtSz-scid (referred to as NOD/scid) mice were bred at the University Health Network/Princess Margaret Hospital. Animal experimentation followed protocols approved by the University Health Network/Princess Margaret Hospital Animal Care Committee. NOD/scid mice 8-13 weeks old were pretreated with 2.75-3.4Gy and antiCD122 antibody before being injected intrafemorally with transduced AML cells at a dose of 200 to 2.87×10̂6 sorted cells per mouse, as previously described²³. Anti-CD122 antibody was purified from hybridoma cell line TM-b1 (generously provided by Prof T. Tanaka, Hyogo University of Health Sciences) and 200 ug injected i.p. following irradiation. Mice were sacrificed at 6.5 to 15 weeks (mean 10 weeks) and bone marrow from the injected right femur and opposite femur and, in some cases, both tibias as well as spleen, were collected for flow cytometry and secondary transplantation. Human engraftment was evaluated by flow cytometry of the injected right femur and non-injected bones and spleen. A threshold of 1% human CD45+ cells in bone marrow was used as positive for human engraftment. For each case, sort purity was integrated with the frequency of LSC in the other fractions in order to estimate LSC contamination and eliminate false positives (LSC+). Mice with greater than 50% CD19+ cells were labeled as normal human engraftment. The mean purity for each fraction was 98.3%. To eliminate false negative results (LSC−), the sensitivity of detection for each fraction was based upon the equivalent of unsorted cells injected (based upon the frequency of the sorted population). Each sorted fraction negative for LSC in vivo represented the equivalent of 6.58×10̂7 unsorted cells (mean). 5×10̂6 unsorted AML cells were confirmed to engraft mice for each sample. CD33 positivity was used to confirm the AML nature of the engraftment. Secondary transplantation was performed by intrafemoral injection of cells from either right femur or pooled bone marrow from primary mice into 1-3 secondary mice pretreated with irradiation and anti-CD122 antibody. For validation of cord blood HSC, 3×10̂3 to 1×10̂5 cells were injected intrafemorally per mouse and human engraftment determined by assessment of human CD45, CD19 and CD33 as previously described³³. Human CFC assays were done as previously described³³.

### Microarray and Bioinformatics Analysis

RNA from cord blood or AML cells was extracted using Trizol (Invitrogen) or RNeasy (Qiagen). RNA was amplified before array analysis by either Nugen (NuGEN Technologies) or in vitro transcription amplification for AML and cord blood, respectively. The in vitro transcription method is an optimized version of the T7 RNA polymerase based RNA amplification published by Baugh et al⁷⁸. Human genome U133A and U133B arrays were used for cord blood and HT HG-U133A arrays for AML samples (Affymetrix). Data was normalized by RMA using either RMA Express ver. 1.0.4 or GeneSpring GX (Agilent). Clustering and heat maps were generated using MeV^{79, 80}. LSC data was clustered using Pearson correlation metric with average linkage. HSC data was clustered using Pearson uncentered metric with average linkage. Gene Ontology (GO) annotation was performed using DAVID Bioinformatics Resources 6.7^{81, 82}.

The LSC-R expression profile was generated by a comparison of gene expression in LSC fractions with those fractions without LSC. The HSC-R expression signature was derived from an ANOVA analysis of probes more highly expressed in HSC1 than all other populations as well as probes more highly expressed in HSC1 and HSC2 than other populations. qRT-PCR confirmation of HSC microarray expression was performed using an ABI PRISM 7900 sequence detection system (Applied Biosystems) and GAPDH to normalize expression.

Gene set enrichment analysis was performed using GSEA v2.0 with probes ranked by signal-to-noise ratio and statistical significance determined by 1000 gene set permutations^{34, 35}. Gene set permutation was used to enable direct comparisons between HSC and LSC results (<7 replicates and >7 replicates, respectively). Median of probes was used to collapse multiple probe sets/gene. For the GSEA analysis of the 110 AML cohort by the LSC-R signature, an LSC-R gene set generated by FDR cutoff of 0.1 was used in order to have >100 probes . . . .

Differentially expressed genes were mapped to known and interologous protein-protein interactions (PPIs) in I2D (Interolog Interaction Database) v1.72 (http://ophid.utoronto.ca/i2d)^{36, 37}, with additional updated PPIs (February 2010) from BioGrid (http://www.thebiogrid.org)⁸³, DIP (http://dip.doe-mbi.ucla.edu)⁸⁴, HPRD (v8; http://www.hprd.org)⁸⁵, IntAct (www.ebi.ac.uk/intact/⁸⁶) and MINT (mint.bio.uniroma2.it/mint/)⁸⁷. Experimental PPI networks were generated by querying I2D with the target genes/proteins to obtain their immediate interacting proteins, and their mutual interaction. Network visualization was performed using NAViGaTOR ver. 2.1.15 (http://ophid.utoronto.ca/navigator)^{37, 88}.

Correlation with Clinical Outcome

All patients in the 160 AML cohort received intensive double-induction and consolidation therapy^{55, 89}. 156 of these patients were enrolled in the AMLCG-1999 trial^{55, 89}. Of the 163 samples, 3 were removed for being peripheral blood or MDS RAEB. Characterization and gene expression profiling of these cohorts is described in Metzeler et al. (GEO accession GSE12417)⁵⁵. The log 2 expression values for each sample were centered to zero mean. The sum of log₂ expression values of the HSC-R or LSC-R probe sets was used as the risk score for each patient. The 160 patients were split into high and low risk groups above and below the median risk score. These risk groups were assessed for prognostication of overall survival and event-free survival in univariate Cox analysis (logrank test) and in multivariate Cox analysis (Wald test). Similarly, the sum of log 2 expression of LSC-R or HSC-R FDR0.05 signature was used to rank the 110 AML cohort (subdivided by cytogenetic risk (GEO accession GSE6891 matrix1)), and chi-squared test applied to the top quartile of samples (highest expression sum). The “phenotypically determined stem cell signature” (FIG. 7c) was derived from a comparison of AML CD34+/CD38− vs AML CD34+/CD38+ cells. This analysis included an additional 7 AML samples that were not included in the generation of the LSC-R data because they had not been functionally validated (Table 15).

### Statistics

Frequency of LSC was determined with a limited dilution analysis and interpreted with the L-Calc software (StemSoft Software Inc). The lower estimate of frequency in cases without negative results was estimated using ELDA (WEHI—Bioinformatics Division)⁹⁰. The HSC-R signature was generated using oneway ANOVA analysis using Tukey HSD post-hoc test and Benjamini-Hochberg multiple testing correction (FDR 0.05) (GeneSpring GX software Agilent). The LSC-R signature was generated using a Smyth's moderated t-test with Benjamini-Hochberg multiple testing correction to compare fractions positive for LSC against fractions without LSC⁹¹. Fisher's exact test was used to determine correlation between LSC-R or HSC-R and complete remission.

### Results:

AML LSC have Heterogeneous Surface Marker Profiles and Frequency

As an initial step to investigate the molecular regulation of LSC, primary human AML patient samples were fractionated into LSC-enriched and LSC-depleted populations to enable further analysis. A xenotransplant model, including the pretreatment of NOD/scid mice with an anti-CD122 antibody (to deplete residual natural killer and macrophage cell activity) and intrafemoral injection of cells, was previously shown to increase the sensitivity of engraftment and detection of stem cells^{18, 23, 24}. Thus, 16 primary human AML samples were sorted into 4 cell populations each based upon surface expression of CD34 and CD38, followed by functional validation in this optimized xenotransplant assay (FIG. 5, see Table 7 for patient and sample data).

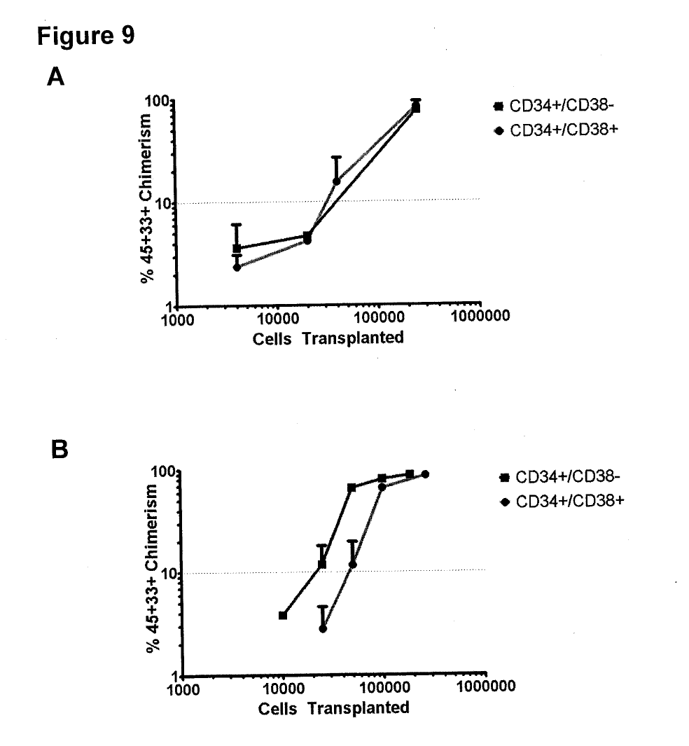

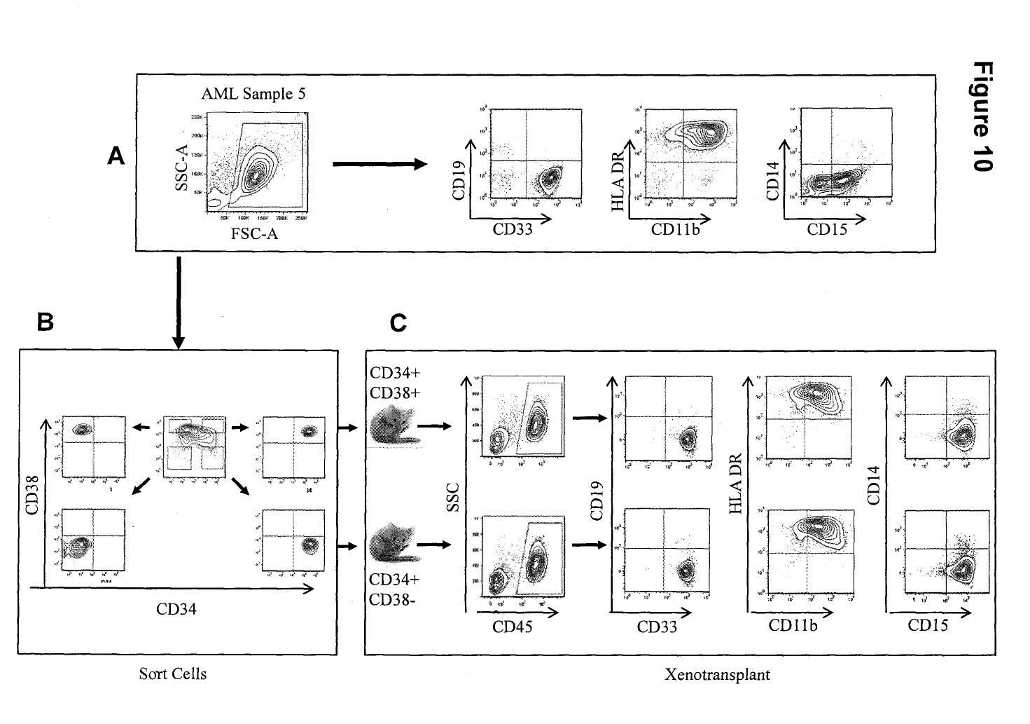

LSC were detectable in each of the four CD34/CD38 AML fractions as determined by human engraftment (≧1% human cells, 8+ weeks after injection) (FIG. 5, Table 8). As expected, LSC were observed in the CD34+/CD38− fraction in each informative case but one; in addition, LSC were also detected in other fractions in the majority of AML samples. The LSC were able to engraft secondary mice, a test of long term self renewal, irrespective of marker profile (Table 9). Additionally, the immunophenotype of the leukemic graft in mice was similar to the primary patient sample and the linear relationship between the number of LSC transplanted and level of human chimerism was the same regardless of the marker profile of the transplanted cells (FIG. 9, 10). This indicates that LSC from different fractions are functionally indistinguishable and can be treated equally in gene expression analysis. In those fractions where LSC were detected the frequency varied from 1/1.6×10³ to 1/1.1×10⁶ cells, as determined by limiting dilution analysis (LDA) in vivo, and was generally highest in the CD34+/CD38− fraction (Table 8). In ten cases the LDA analysis was repeated and the results were highly consistent among replicates. Further, an estimate of the absolute number of LSC contributed by each fraction revealed that the majority of LSC are in the minor CD34+/CD38− fraction in 50% of the patients, and in the CD34+/CD38+ fraction in the other 50% (Table 10, 11). Thus, using an optimized xenograft model, it can be concluded that AML LSC represent a minor population that can be reproducibly purified and they are able to self-renew and re-establish the AML hierarchy in xenograft models. Collectively, these data provide strong evidence that AML is organized as a hierarchy that follows a CSC model.

### Transcription Profiling of Functionally Defined LSC

To gain insight into the molecular regulation of LSC, each of the functionally validated fractions derived from all 16 primary human AML samples were subjected to global gene expression analysis (FIG. 5). Two assumptions were made. First, that an LSC specific transcriptional profile will contain at least some genes that govern the stem cell state. Second, that comparison of closely related cell fractions that differ only by the absence or presence of LSC will reveal LSC specific gene expression even though the actual LSC frequency remains relatively low. There is ample precedence for both assumptions from many gene expression studies of normal HSC, where subsequent studies have proven the HSC specific function of the differentially expressed genes²⁵⁻²⁸. Since the goal was to generate an LSC-related gene profile (LSC-R) bioinformatic analysis was undertaken to compare global gene expression of the 25 LSC enriched fractions with the 29 fractions in which LSC were absent (Table 12 for top 80 array probe list). The LSC-R signature, comprised of genes more highly expressed in LSC enriched populations, with a false discovery rate (FDR) of 0.05, consists of 42 genes (48 probes sets) (probe sets listed in Table 5 and genes listed in Table 6). This represents a common signature, as it was generated from AML samples that possessed a variety of karyotypic alterations and FAB subtype. Prior reports of LSC specific gene expression used simple comparisons of LSC to HSC²⁹⁻³¹, phenotypically defined cell populations (where both may have contained LSC as the data herein establishes)³² or used a small patient cohort⁵. Comparison of both the LSC-R and a normal HSC signature (described below) with prior work,^{2, 31, 32, 35} is shown in Example 7. By contrast, the approach taken here resolves these problems by focusing the analysis only on a large number of functionally validated LSC-enriched versus non-LSC AML populations resulting in the identification of a novel LSC-specific gene signature (probe sets listed in Table 5, genes listed in Table 6).

### Functionally Defined HSC Related Transcription Profiles

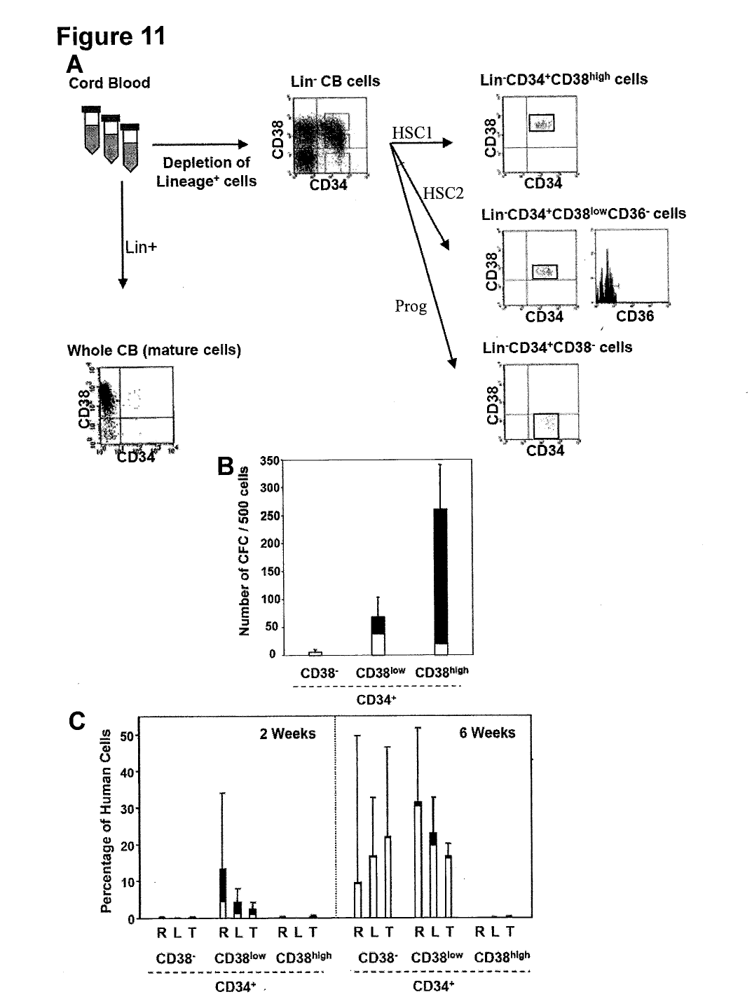

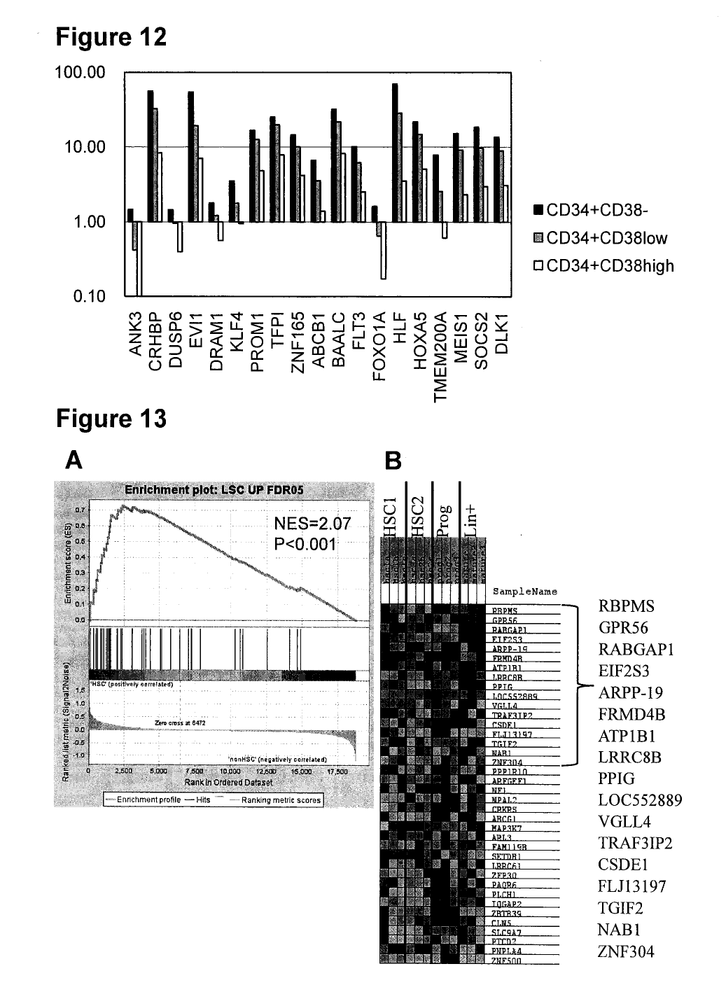

LSC and HSC both possess canonical stem cell functions such as self renewal and maturation processes that result in progeny that lack stem cell function¹. However it is not known if human LSC utilize molecular mechanisms also employed by HSC or if they are governed through unique pathways. If gene expression programs are shared between LSC and HSC, there is a high likelihood that some will govern common stem cell functions, and such a comparison provides the first step in their identification To determine the gene expression profile of HSC, gene expression in human cord blood CD34+/CD38− (HSC1), CD34+/CD38lo/CD36− (HSC2), and CD34+/CD38+ (progenitor) cells as well as lineage positive (mature) cells were examined (FIG. 11). It has been previously reported that the HSC2 fraction contains a lower frequency of HSC than HSC1 and a novel class of repopulating cells termed R-SRC³³. An HSC-related profile (HSC-R) was generated based on transcript enrichment in HSC fractions (FIG. 5a, FIG. 11, Table 14). The HSC and progenitor enrichment in each fraction was validated by in vitro colony formation and in vivo xenograft assays (FIG. 11). The HSC-R signature of genes with higher expression in HSC fractions (FDR 0.05) consists of 121 genes (147 probes sets (Table 14). The differential expression of 19 genes was validated by qRT-PCR (FIG. 12) In order to facilitate gene ontology (GO) analysis, larger lists using an FDR cutoff of 0.10 were also used: an FDR0.1 HSC signature is enriched in 63 GO categories, including the 5 GO categories in which the FDR0.10 LSC signature is enriched.

### LSC Express an HSC Gene Expression Profile

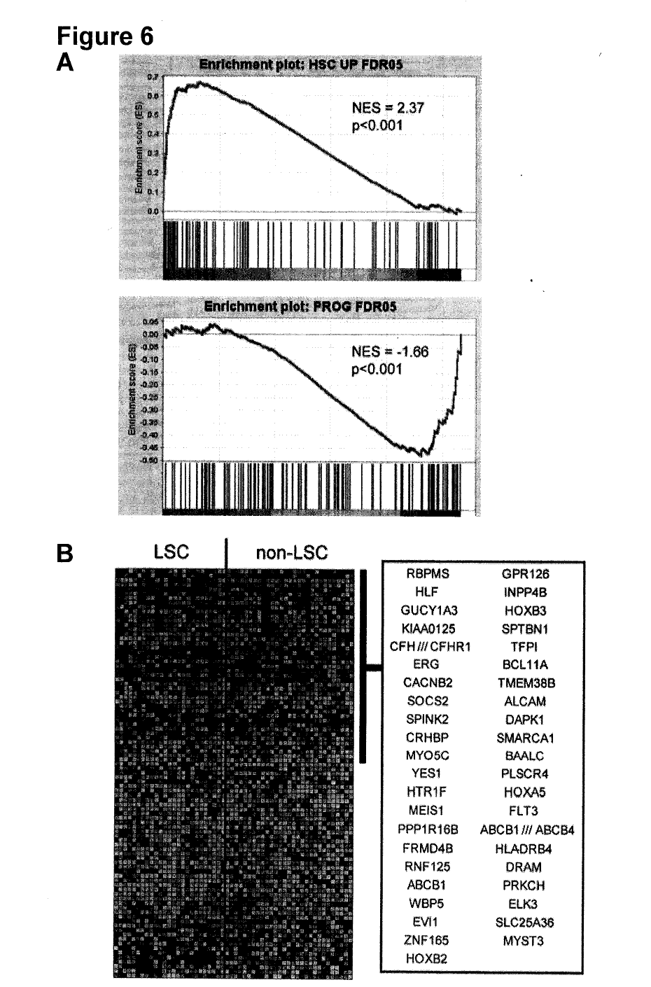

The LSC-R and HSC-R gene expression profiles were examined for common expression patterns. Gene Set Enrichment Analysis (GSEA), a threshold-free method of comparing gene expression between independent datasets, was used to compare the expression profiles and found enrichment of the HSC-R gene signature in the LSC-R profile (p<0.001) (FIG. 6A top panel, 6B)^{34, 35}. Conversely, the LSC-R signature was found to be enriched in the HSC-R expression profile (p<0.001) (FIG. 13). Forty-four leading edge genes termed the “core enriched HSC/LSC” genes (CE-HSC/LSC), drive the GSEA enrichment of the HSC-R signature in the LSC gene expression data and represent HSC genes that are also differentially expressed in LSC; of these 18 have previously been implicated in stem cell regulation, oncogenesis, or both, including ABCB1(MDR1), MEIS1, ERG, HLF, EVI1 and homeobox genes (FIG. 6B; see Example 8 for a complete description of these genes). A subset is included in Table 13.

To identify the core pathways that these genes might predict, a stem cell protein-protein interaction network from the CE-HSC/LSC genes was generated, consisting of direct protein-protein interactions as well as proteins that link CE-HSC/LSC proteins using the I2D protein interaction database^{36, 37}. The full network is available in NAViGaTOR 2.0³⁷ XML file format at http://www.cs.utoronto.ca/˜juris/data/NatMed10/. Further, a gene list as well as protein network representing more highly expressed genes common to normal lineage-committed progenitors was generated. The CE-HSC/LSC protein interaction network shows significant enrichment of multiple pathways separate from the progenitor network, including Notch and Jak-STAT signaling, which are implicated in stem cell regulation, thereby supporting the stem cell nature of the HSC and LSC-related gene profiles³⁸⁻⁴⁴. To gain further insight into the gene expression programs preferentially active in LSC, this data was compared with previously generated human and murine gene sets derived from stem, progenitor and mature cell populations as well as embryonic stem cells (ESC)^{25, 28, 45-51}. In a comparison of gene expression between LSC and non-LSC fractions by GSEA, LSC-R gene expression positively correlated with pre-existing primitive cell gene sets such as HSC genes and genes shared between HSC and lineage-committed progenitor cells, and negatively correlated with gene sets derived from more differentiated cells such as late lineage-committed progenitor and mature blood cells (FDR q≦0.05; see Example 9 for further description)^{25, 28, 45}. As well, the normal common lineage-committed progenitor-related gene list negatively correlated with genes more highly expressed in LSC fractions than with non-LSC (p<0.001) (FIG. 6A bottom panel). In a similar analysis, LSC were not enriched for ESC modules or ESC gene expression sets compared to non-LSC, unlike what was previously observed for murine MLL-induced leukemia LSC⁴⁶⁻⁵² (FDR q>0.05). Thus, an HSC expression program, and not a common lineage-committed progenitor or ESC expression pattern, is preferentially expressed in LSC compared to more mature leukemic cells.

### LSC and HSC Gene Expression Signatures Predict Outcome of Leukemia Patients

To investigate whether there is a correlation between these LSC-R and HSC-R gene signatures and clinical outcomes in AML patients, a pre-existing set of AML gene expression profiles were interrogated⁵³⁻⁵⁵. As discussed later, this approach assumes that, since a hallmark of AML is altered growth and blocked differentiation, some components of stem cell gene expression programs will persist in leukemic blasts. In their study, Valk et al. examined global gene expression in leukemic blasts from 285 AML patients and identified 16 distinct groups by unsupervised cluster analysis⁵³. In general, clustering was driven by the presence of gross chromosomal alterations and known point mutations. When the genes that define each cluster were examined in the LSC-R and HSC-R profiles, a significant enrichment for a number of clusters was found. Generally, the LSC-R and HSC-R profiles produced similar results in the enrichment of the clusters and correlated positively with clusters characterized by FLT3-ITD or EVI1 over-expression, molecular markers that indicate a poor prognosis^{53, 56-58}. They correlated negatively with clusters that have good prognosis, including karyotypes such as t(15;17) and inv(16) although 11q23 MLL was also in this group⁵³. Recently, 110 of these AML samples were stratified into ‘poor’ or ‘good’ prognostic risk groups, based upon cytogenetic alterations, and new gene expression data was generated⁵⁴. Higher expression of the LSC-R or HSC-R signatures was able to predict poor prognostic risk patients in this data set (p=0.0125 and p=0.001 respectively). Further, enrichment analysis identified subsets of LSC-R and HSC-R genes that correlate with poor cytogenetic risk groups (FIG. 14). This subset of the HSC-R signature has considerable overlap with the shared CE-HSC/LSC gene list (21 of 32 genes) (FIG. 6c, 14). Overall, these findings support the validity of the stem cell expression profiles and demonstrate that AML with worse prognosis express stem cell-related genes more highly than less aggressive AML samples. Furthermore, they establish the feasibility of using an LSC or HSC signature as a biomarker to stratify patients through analysis of their bulk blast populations.

To validate the clinical relevance of stem cell gene expression in leukemia, a second cohort of 160 cytogenetically normal (CN) AML patients were examined for whom gene expression and outcome data was available⁵⁵. CN AML represents approximately 45% of all AML subtypes and is an intermediate risk category^{57, 58}. The LSC-R or HSC-R gene signature was used to divide these patients into 2 equal groups based upon the median expression of the respective signature in bulk AML bone marrow cells. There was significant negative correlation between the rate of complete remission and high expression of the LSC-R signature (p=0.0054, n=158), while negative correlation with the HSC-R signature approached significance (p=0.073, n=158). Both signatures negatively significantly correlated with overall survival (LSC p=5.2×10̂−6, HR=2.4 (95% Cl 1.6-3.6); HSC p=1.8×10̂−5, HR=2.3 (95% Cl 1.6-3.4)) (FIG. 7a) and event-free survival (LSC p=2.5×10̂−7, HR=2.5 (95% Cl 1.8-3.7); HSC p=8.9×10̂−6, HR=2.2 (95% Cl 1.5-3.2)) (FIG. 7b). It is noteworthy that a signature generated using phenotypic stem cell markers alone without functional determination of LSC fractions was not prognostic (p=0.81, HR=1, Table 15), supporting the requirement for functional validation of LSC populations (FIG. 7d). Thus, this data demonstrates that high expression of stem cell expression signatures directly predict patient survival in CN AML and, therefore, variation in stem cell expression programs among patients is highly correlated to heterogeneity in disease outcome.

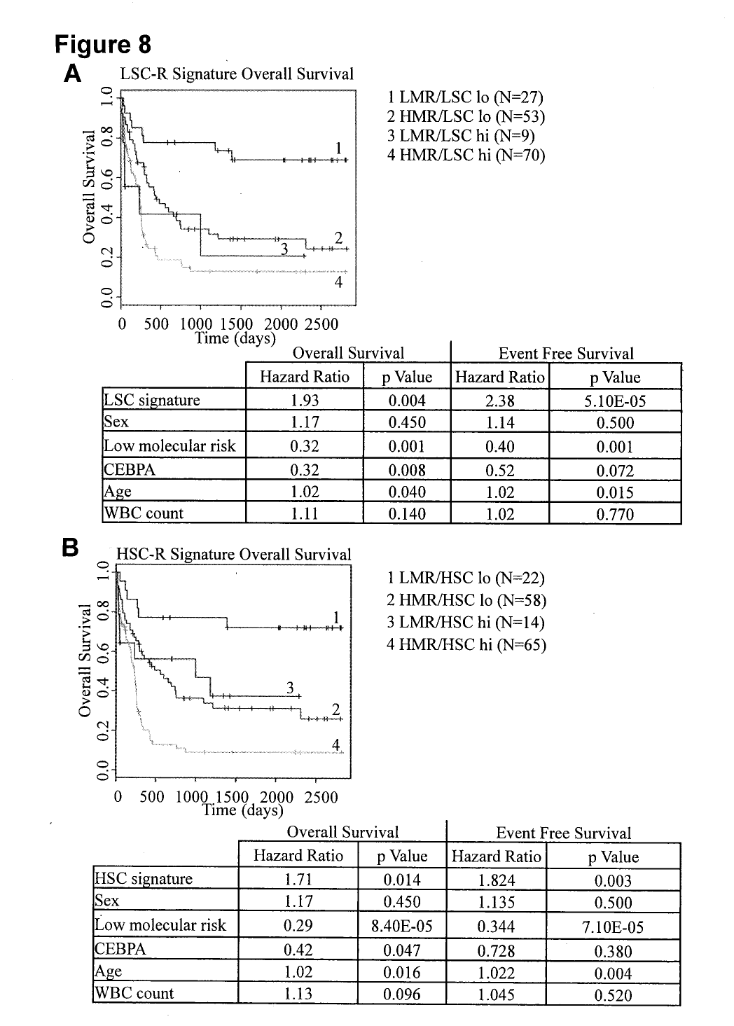

CN AML patients lack gross genomic changes making it difficult to identify a prognostic biomarker. However, there has been much effort to use mutational status of specific genes to determine prognosis⁵⁷⁻⁶¹. Recently, FLT3ITD status and NPM1 mutational status have been combined to designate low molecular risk (NPM1mut FLT3ITD−) (LMR) and high molecular risk (FLT3ITD+ or NPM1wt FLT3ITD−) (HMR) groups^{57, 60, 61}. Patients with LMR AML, who generally account for approximately 35% of CN-AML, have favorable prognosis and are offered standard treatment, however there is still heterogeneity in outcome^{57, 60, 61}. Multivariate analysis was used to demonstrate that the LSC-R and HSC-R signatures could predict outcome independently of known molecular prognostic factors such as molecular risk status and CEBPA (FIG. 8) (See Example 10 for an analysis with FLT3 and NPM1 as independent factors)^{57, 60-62}. Subdividing the 160 CN AML cohort by molecular risk status, it was observed that each stem cell signature identified patients with worse survival in both the HMR subset (LSC-R p=0.003, HR=1.9 (95% Cl 1.2-2.9); HSC-R p=0.00023, HR=2.2 (95% Cl 1.4-3.4)) and the LMR subset (LSC-R p=0.0033, HR=4.5 (95% Cl 1.5-13); HSC-R p=0.021, HR=3.3 (95% Cl 1.1-9.7)) (FIG. 8). Patients with high LSC-R signature represented only 25% of the LMR group and yet accounted for approximately 50% of the LMR patients that did not survive. As the patients in the LMR group were considered to have favorable prognosis, approximately only 10% of the patients in this cohort received a bone marrow transplant. Thus, the LSC-R and HSC-R signatures can be used to stratify patients currently identified as low risk into those who do well with standard therapy and those who could benefit from more intensive therapy, including stem cell transplant.

To determine the robustness of the clinical correlation, the prognostic value of the LSC-R signature was examined in an additive analysis (FIG. 7c). Starting with the highest ranked LSC probe in the LSC-R gene expression profile, the correlation with outcome was determined (as measured by p value, Table 16) after successive addition of each ranked probe. Correlation with overall survival was greatest with the top 35 probes. Beyond that, the correlation decreased but was still significant at 1000 probes (p=0.04). These findings indicate that the stem cell profile is consistently of prognostic significance and that this correlation is not driven by a single, or very few, genes or pathways. Collectively, these data provide strong evidence that stem cell properties influence patient survival outcomes.

### Discussion

This data provides human HSC and LSC-specific gene expression signatures derived from multiple sorted cell fractions where both HSC and LSC content was contemporaneously assayed by in vivo repopulation. LSC and HSC share a core transcriptional program that, when taken together, reveals components of the molecular machinery that govern stemness. Since both signatures show strong prognostic significance predicting AML patient outcome, the data establishes that determinants of stemness influence clinical outcome. These findings have two important implications on the role of stem cells in cancer. First, the firm linkage between LSC and HSC signatures and the ability of these signatures to predict survival, a seminal cancer property, provide strong evidence that LSC defined on the basis of functional stem cell properties are distinct and clinically relevant cells present in the leukemic clone. Although the validity of the CSC model continues to be contested for many tumour types, this data supports the contention that LSC are discrete cell types and not artifacts of experimental xenograft models or clinically unimportant^{17, 20, 63-66}. Second, the approach that has been taken in AML provides a paradigm for assessing both the identity and clinical relevance of LSC and CSC from other leukemias and solid tumours, respectively. A well validated and sensitive xenograft assay is essential since only functionally validated populations showed clinical relevance, while signatures derived from phenotypically defined populations did not. Furthermore, the finding of LSC clinical relevance predicts that therapies targeting LSC should improve survival outcomes and that xenograft models based on primary AML engraftment should be used for preclinical evaluation of new cancer drugs.

The identification of shared transcriptional profiles in LSC and HSC strongly predicts that these components of the molecular machinery must play a role in the establishment and maintenance of the stem cell state. Indeed biological studies have clearly established that LSC and HSC share a number of properties including quiescence, niche dependence, and self renewal¹. Although this study was not designed to determine the mechanism whereby these genes govern the stem cell properties, it can be inferred that many must have an important role. Genes such as EVI-1, MEIS1, HOXB3, and ERG as well as the pathways identified from network analysis are well known as critical regulators of normal murine and/or human HSC function⁶⁷⁻⁷⁰. Moreover, many genes such as EVI-1, ERG, FLT3 and BAALC are also associated with poor prognosis in AML^{58, 71}. As each is present in the shared stem cell gene profile, it is speculated that their value as a highly significant prognostic indicator derives from their role in governing stem cell function. Collectively, the identification of so many (eighteen) known stem cell and leukemia genes within the transcriptional profile provides confidence that many of the remaining genes not previously associated with the stem cell state are indeed functionally relevant in human LSC and HSC. The shared stem cell profile also adds to the discussion and controversy regarding the cell of origin for AML and whether LSC derive from the transformation of HSC or committed progenitors^{1, 16, 72-75}. GSEA showed that LSC were only enriched for HSC programs and not from progenitor or embryonic cell programs, pointing to their close relationship.

The prognostic value that was found in the LSC and HSC signatures is of significant clinical importance in a disease like AML where a large proportion of patients are cytogenetically normal. Gross genomic changes (e.g. chromosomal translocations) cannot be used to guide therapy, but the mutational status of a small number of genes is now widely employed to stratify LMR patients toward less aggressive treatment compared to HMR patients^{57, 60, 61}. It is particularly noteworthy that the LSC signature clearly identified a large subset (45%) of patients in the LMR group that had poor long term survival. Such patients might benefit from more aggressive therapy. It is somewhat counterintuitive that an LSC/HSC signature should be present in the leukemia blasts (i.e. non-LSC) of a patient with poor outcome. It is possible that the higher expression of a signature simply reflects a higher proportional content of LSC, as suggested previously¹², and such cells are harder to eradicate making patient survival shorter. However even in the peripheral blood of AML patients with the highest frequency of LSC only 1 in 500 to 1000 cells is an LSC making it highly unlikely their gene expression was detected. Alternatively, it is well known that as normal HSC maturation occurs there is an essential substitution of stem cell functions (including self renewal, quiescence, DNA damage response, apoptosis) by differentiation programs. In AML, differentiation is perturbed and abnormal but also highly variable between genetic and morphological subtypes⁷⁶. Additionally, human and murine studies have clearly shown that the self renewal capacity of LSC is abnormal resulting in massive LSC expansion compared to normal HSC^{1, 64, 77}. It is speculated that there is similar variation in the uncoupling of stem cell functions and maturation programs. This data argues that when this dissociation is poor the stem cell programs will persist in bulk leukemia blasts, while in other samples there is a more rigid demarcation between the LSC and non-LSC similar to normal HSC development. The reason the blasts in the former example lack actual LSC function is that any individual blast will only possess a limited repertoire of the full program but since RNA is collected from a large cell dose the full program will be uncovered. If this explanation is correct the greater retention of residual stem cell properties in all cells of the leukemic clone is reflective of an LSC whose stem cell properties are more deregulated resulting in disease progression, treatment failure and shortened survival. More broadly, this data points to the importance of developing LSC biomarkers to contribute to personalized cancer therapy and the need to identify therapeutic targets that will target all leukemic cells in the clone including the LSC.

### Example 7

The relationship of the LSC-R and HSC-R gene profiles to previously elucidated human LSC-associated gene expression data was examined. Four previous studies assessed LSC global gene expression. These involved either a comparison of LSC to HSC (AML vs normal, CD34+/CD38− cells)^{55, 56} or LSC to more differentiated AML cells in small patient cohorts (AML CD34+/CD38− vs CD34+/CD38+ cells)^{57, 58}. In one latter case, the LSC nature of each fraction was not functionally validated⁵⁸ and, as shown here and as others have shown, the use of CD34 and CD38 to identify stem cell fractions without concomitant functional analysis can mislabel the stem cell nature of sorted cell fractions.

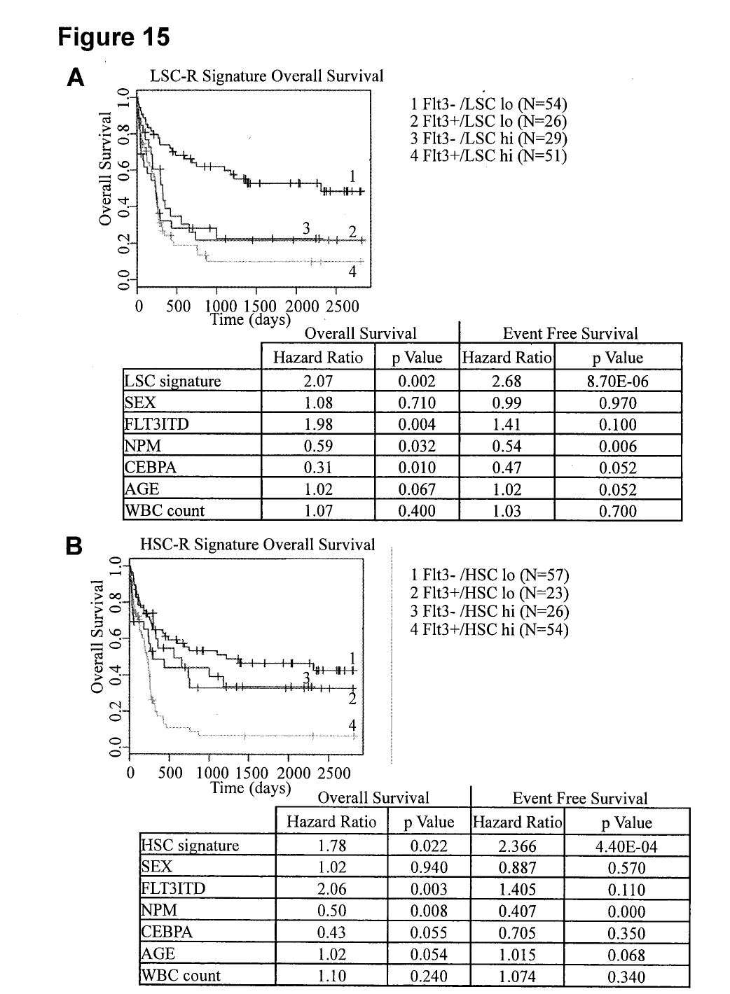

First, of the studies that compared LSC-enriched populations to non-stem cell enriched AML cells, no correlation with the LSC list generated by Gal et al based upon phenotypically defined populations (AML CD34+/CD38− vs CD34+/CD38+ cells)⁵⁸ was found. FIG. 15a-b). As there was no functional validation, the phenotypically determined non-LSC (CD34+/CD38+) samples likely included LSC in some patients, compromising the data analysis. However, there was a negative correlation of the genes underrepresented in LSC with both the LSC-R and HSC-R data sets. This suggests that the CD34+/CD38+ cell fractions included a mixed population, resulting in higher expression of genes linked to maturation than in the CD34+/CD38− population. In the second study of LSC to non-LSC AML populations, Ishikawa et al. used a cohort of 4 samples with 2 populations each to identify a small number of genes⁵⁷. In this case, there is some correlation with LSC-R and HSC-R although, critically, the LSC-R does not positively correlate with their LSC up regulated gene set nor does HSC-R negatively correlate with their down regulated LSC gene set (FIG. 15 a-b). This suggests that while this study was successful in identifying some LSC-related stem cell genes, it was limited by small sample size and the gene expression variability inherent in cancer samples.

The LSC-R and HSC-R gene expression data here was then compared with the gene sets identified in the two studies that contrasted the gene expression of LSC-enriched populations (AML CD34+/CD38− cells) with HSC-enriched populations (normal CD34+/CD38− cells)^{55, 56}. While a comparison of gene expression of LSC against HSC may identify genes deregulated in LSC, it does not take into account the expression of leukemia associated genes that are independent of the stem cell nature of the populations. When applied to the LSC-R and HSC-R data, the results are the same: in both cases, the genes more highly expressed in LSC vs HSC were negatively correlated with the LSC-R and HSC-R stem cell related expression data while the genes with lower expression in LSC vs HSC were positively correlated with the LSC-R and HSC-R stem cell related expression (FIG. 15 c-d) AML cells aberrantly express mature cell markers, even in the primitive cell population, and therefore also likely express multiple mature cell gene expression programs, even at only a low level. Thus, the list of genes with higher expression in LSC vs HSC likely includes genes normally highly expressed in mature cells that are aberrantly expressed in the AML CD34+/CD38− population. These gene lists are therefore found to correlate with the non-LSC and non-HSC genes in the LSC-R and HSC-R stem cell profiles developed here as they are generally highly expressed in differentiated cells. For example, the LSC list by Saito et al., contains genes expressed in more mature cells such as MPO, CD93, CD97, CD24, and HCK⁵⁶. This analysis supports the experimental design of Saito et al as one aim was to identify surface markers uniquely expressed in LSC and not HSC. Further, as the frequency of stem cells is substantially higher in the CD34+/CD38− compartment of normal cord blood and bone marrow compared to AML, it is not surprising that a comparison of these populations would identify stem cell genes as more highly expressed in the normal HSC population than the equivalent LSC population, as occurred in these two studies. Thus, these results indicate that the comparison of gene expression in LSC-enriched populations with HSC-enriched populations, as carried out in these two studies, succeeded in identifying genes aberrantly expressed in LSC. Critically, however, this strategy resulted in exclusion of most of the common stem cell genes as LSC-related genes.

Overall, these analyses establish the necessity in CSC gene expression studies to functionally validate each stem cell population in a sensitive xenograft model. Further, they highlight the requirement to compare CSC populations against non-CSC cancer populations, as opposed to CSC vs normal populations, when the goal of the study is to provide insight into the entire stem cell-related gene expression program present in CSC.

### Example 8

The HSC-R genes enriched in GSEA analysis of the LSC expression profile (CE-HSC/LSC) represent a group of stem cell related genes that are active in both stem cell populations compared to their respective non-stem cell fractions (FIG. 6d). Approximately half of these genes (18/44) have been implicated in stem cell function or leukemogenesis, or both (eg. EVI1):

ABCB1 (ATP-binding cassette, sub-family B (MDR/TAP), member 1; MDR1) acts as a drug transport pump and imparts a multidrug resistant phenotype to cancer cells^{1, 2}. Further, the high expression of ABCB1 in stem cells provides a mechanism for the high efflux of dyes, which can be used to isolate a ‘side population’ of cells that are enriched for stem cells^{3, 4}. Additionally, ABCB1 expression negatively correlates with treatment response in leukemia⁵.

ALCAM (activated leukocyte cell adhesion molecule; CD166) is a cell surface molecule identified as a marker for the enrichment of colon cancer stem cells⁶. ALCAM has been implicated in cancer; for example, increased expression of ALCAM is a prognostic marker for poor outcome in pancreatic cancel^{7, 8}.

BAALC (Brain and acute leukemia gene, cytoplasmic) was identified in an attempt to isolate genes differentially expressed in AML+8 compared to cytogenetically normal AML⁹. High expression of BAALC correlates with poor outcome in leukemia^{10, 11}. BAALC is preferentially expressed in CD34+ primitive cells and expression is down-regulated upon cell differentiation¹².

BCL11A (B-cell CLL/lymphoma 11A (zinc finger protein)) is implicated in leukemogenesis as a target of chromosomal translocations of the immunoglobulin heavy chain locus in B-cell non-Hodgkin lymphomas¹³.

DAPK1 (Death-associated protein kinase 1) is a serine/threonine kinase gene involved in regulating apoptosis¹⁴. Decreased expression of DAPK1 has been implicated in both inherited and sporadic chronic lymphocytic leukemia¹⁵.

ERG (Ets-related gene), a transcription factor required for normal adult HSC function, is rearranged in human myeloid leukemia and Ewing's sarcoma¹⁶⁻¹⁸. Additionally, over-expression of ERG is observed in leukemia and associated with poor patient outcome in AML with normal karyotype^{10, 19, 20}.

EVI1 (Ecotropic viral integration site 1) is a nuclear transcription factor implicated in regulation of adult HSC proliferation and maintenance²¹. Excision of EVI1 in mice results in a decrease of HSC frequency while over-expression results in greater self-renewal. Additionally, EVI1 plays a role in leukemogenesis²². It is a target of translocation events in human leukemia, for example, generating the fusion protein RUNX-EVI1 as a result of t(3;21)(q26;q22). High expression of EVI1 is associated with poor patient outcome^{22, 23}.

FLT3 (Fms-like tyrosine kinase 3; Stem cell tyrosine kinase 1, STK1; Flk-2) is a receptor tyrosine kinase expressed in primitive hematopoietic cells that has been implicated in the regulation of HSC^{16, 24-26}. Mutation of FLT3 is a strong prognostic indicator in CN-AML associated with poor outcome²⁷⁻²⁹.

HLA-DRB4 (major histocompatibility complex, class II, DR beta 4) has been linked to increased frequency of leukemia. For example, it is a marker for increased susceptibility for childhood ALL in males³⁰.

HLF (Hepatic leukemia factor), a leucine zipper gene, is involved in gene fusions in human leukemia as well as acting as a positive regulator of human HSC^{31, 32}.

HOXA5 (homeobox A5), along with HOXB2, HOXB3 and MEIS1 is a homeobox gene and is hypermethylated in leukemia³³. The hypermethylation of HOXA5 is correlated with progression of CML to blast crisis³⁴.

HOXB2 (homeobox B2) is a member of the HOX gene family. Increased HOXB2 expression is associated with NPM1 mutant CN AML, supporting a correlation between altered HOX expression and NPM1 mutation³⁵.

HOXB3 (homeobox B3) is expressed in a putative HSC cell population of CD34+ cells³⁶ and has been shown to regulate the proliferative capacity of murine HSC when mutated along with HOXB4³⁷. Furthermore, HOXB3 can induce AML in mice when expressed along with MEIS1³⁸.

INPP4B (inositol polyphosphate-4-phosphatase, type II, 105 kDa) has been implicated as a tumour suppressor gene, supported by the observation of common loss of heterozygosity of the INPP4B locus correlating with lower overall patient survival³⁹.

MEIS1 (Myeloid ecotropic viral integration site 1 homolog, Meis homeobox 1) is a homeobox gene that is highly expressed in MLL rearranged leukemias^{40, 41}. It has been shown to transform hematopoietic cells when co-expressed with genes such as HOXB3, HOXA9 and NUP98-HOXD13 and acts to regulate LSC frequency in a murine MLL leukemia model^{38, 42-44}. Further, it has recently been shown to regulate HSC metabolism through Hif-1alpha⁴⁵.

MYST3 (MYST histone acetyltransferase (monocytic leukemia) 3; MOZ) is a target of the t(8;16)(p11;p13) translocation commonly observed in M4/M5 AML⁴⁶. It is a transcriptional activator and has histone acetyl-transferase activity⁴⁶. As well, homozygous knockout of Myst3 resulted in HSC defects, indicating that it is the required for HSC function⁴⁷.

SPTBN1 (spectrin, beta, non-erythrocytic 1) is a cytoskeletal protein identified as a fusion partner of FLT3 in atypical chronic myeloid leukemia⁴⁸.

YES1 (v-yes-1 Yamaguchi sarcoma viral oncogene homolog 1) is a member of the SRC family of kinases and, like SRC, is ubiquitously expressed. YES1 expression was shown to be enriched in murine HSC, ESC and NSC⁴⁹. YES1 is implicated in maintaining mouse embryonic stem cells in an undifferentiated state⁵⁰. Furthermore, YES1 was found to be amplified in gastric cancer⁵¹.

### Example 9

Prior studies have generated normal human and murine hematopoietic gene signatures for populations enriched for stem, progenitor and mature cells. The overlap between the stem cell expression profiles shown here with 3 pre-existing stem cell expression sets available in the Molecular Signatures Database (MSigDB)⁵²⁻⁵⁴ using GSEA were examined. First, a human stem cell gene set, developed by Georgantas et al 2004, compared only CD34+ cells split into 2 populations consisting of stem cell enriched (CD34+/CD38− cells from bone marrow, cord blood and mobilized peripheral blood) and a progenitor enriched fraction (CD34+4/[CD38/Lin]+)⁵². This gene set (“HEMATOP_STEM_ALL_UP”) was enriched in both of the HSC-R and LSC-R expression profiles (FDR q<0.05), supporting the stem cell nature of the expression signatures described herein.

Next, a murine gene set representing genes more highly expressed in an HSC population than in a multipotent progenitor (MPP) population (Rhlo/Sca-1+/c-kit+/lin−/lo vs Rhhi/Sca-1+/c-kit+/lin−/lo) were examined⁵³. The MPP in this case represents a progenitor population that can generate both lymphoid and myeloid cells but not reconstitute beyond 4 weeks. This HSC vs MPP list (“PARK_HSC_VS_MPP_UP”) was enriched for in our LSC-R and HSC-R expression profiles (FDR q=0.03 and 0.04, respectively). This further supports the normal hematopoietic gene expression data and indicates that AML LSC preferentially express an HSC program, not an MPP program, compared to non-LSC stem cell populations.

Finally, the 24 murine gene sets generated by Ivanova et al. 2002 available in MSigDB were examined⁵⁴. These were generated by examining gene expression in murine stem cell, lineage committed progenitor and mature blood cells from both adult bone marrow and fetal liver and comparing multiple combinations of populations. In the case of adult bone marrow, both long-term and short-term HSCs were isolated (LT HSC and ST HSC, respectively). In general, the LSC-R and HSC-R profiles were enriched for gene sets from primitive cell populations and were negatively correlated with those derived from differentiated populations (“late progenitor” list and “mature” cell list). As expected, the HSC-R expression data correlated with the combined LT and ST HSC gene list (“HSC” FDR q=0.01) and weakly with the LT HSC list alone (FDR q=0.09). However, the HSC-R did not significantly correlate with the ST HSC gene set (FDR q=0.44). Since a ST HSC has not yet been isolated in the human system, this suggests two possible explanations, among others: that the ST HSC does not exist in humans or that the ST HSC gene expression program is unique and undetectable in our sorted population that contains all forms of human HSC. Examining the human LSC-R profile, there is enrichment of the genes in common to primitive cells (“HSC and progenitors”), a weak correlation with the murine LT HSC set (FDR q=0.14) but no correlation with the shared LT and ST stem cell (“HSC”) set (FDR q=0.45). This implies that LSC may preferentially express the gene programs expressed in murine primitive cells as well as, potentially, a subset of the programs specific for LT HSC, although these analyses may suffer from interspecies differences.

Overall, these analyses support the conclusion that HSC-related gene programs and not progenitor or mature gene programs are expressed in AML LSC compared to leukemic blast cells.

### Example 10

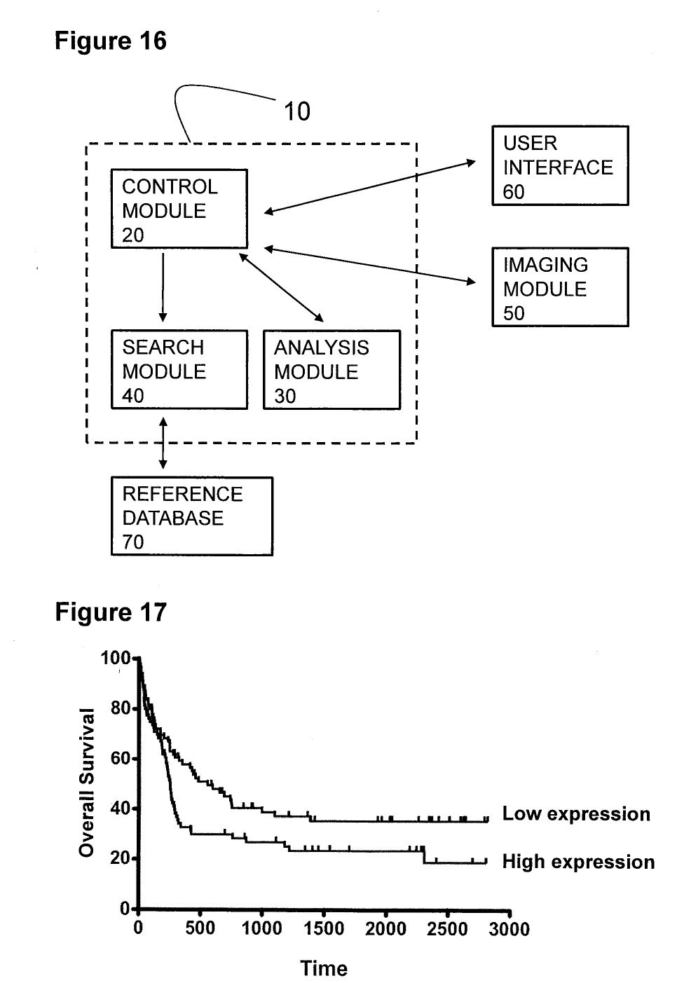

The FLT3ITD mutation is a strong prognostic indicator of poor outcome in cytogenetically normal AML²⁷⁻²⁹. Multivariate analysis demonstrated that the LSC-R and HSC-R signatures could predict outcome independently of known molecular prognostic factors such as FLT3ITD status, NPM1 mutation and CEBPA (FIG. 16)²⁹. Subdividing the 160 AML cohort by FLT3ITD status, it was found that stem cell signature gene expression was able to identify patients with worse outcome in each subset. The LSC-R signature was able to predict patients with worse outcome in the FLT3ITD− patients (p=0.00035, HR 2.8 (95% Cl 1.6-5.2) but not as effectively in the FLT3ITD+ patients (p=0.15, HR 1.5 (95% Cl 0.87-2.6) (FIG. 15). Conversely, the HSC-R signature is able to identify patients with worse outcome in the FLT3ITD+ group (p=0.0013, HR 2.6 (95% Cl 1.4-4.9) and not as successfully in the FLT3ITD− subset (p=0.15, HR 1.6 (95% Cl 0.85-2.9) (FIG. 15). Thus, the stem cell gene signatures are prognostically significant independently of other common prognostic factors.

### Example 11

### Determination of a Threshold

The expression values and clinical outcome data for the a group of normal AML such as the 160 cytogenetically normal AML samples used in the primary study will be used as a test group in an analysis to determine the optimal threshold of expression for the stratification of new patients into poor or good prognostic groups in the clinic.

### Example 12

Individuals who present or are suspected of having a hematological cancer will provide a blood sample. The white blood cell fraction will be tested for the expression of two or more genes listed in Tables 2, 4, 6, 12 and/or 14 or for example two or more CE-HSC/LSC genes such as those listed in tables 13 and 19. The expression values will be scaled (e.g. normalized) to a standard (e.g. using experimental controls) and then compared to a threshold value to determine poor or good prognosis prediction.

### Example 13

A prognostic analysis as conducted as was done in FIG. 7A was repeated for a combination of 2 probe sets from the LSC signature genes. Expression levels were significantly correlated with overall survival in the 160 AML cohort. The p value is 0.0293 and the hazard ratio is 1.53. The porbesets were 214252_s_at and 212676_at. The gene expression levels detected by these probesets are CLN5 and NF1.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
LSC probe set (25)
SEQ ID NO: 1-280

TABLE 2
LSC gene signature (25)

| | | | | | Representative |
|---|---|---|---|---|---|
| | Gene | | Entrez | | Public ID NCBI |
| Probe Set ID | Symbol | Gene Title | Gene ID | UniGene ID | Accession |
| 201242_s_at | ATP1B1 | ATPase, Na+/K+ | 481 | Hs.291196 | BC000006 |
| | | transporting, beta 1 | | | |
| | | polypeptide | | | |
| 201243_s_at | ATP1B1 | ATPase, Na+/K+ | 481 | Hs.291196 | NM_001677 |
| | | transporting, beta 1 | | | |
| | | polypeptide | | | |
| 201702_s_at | PPP1R10 | protein phosphatase 1, | 5514 | Hs.106019 | AI492873 |
| | | regulatory (inhibitor) | | | |
| | | subunit 10 | | | |
| 204028_s_at | RABGAP1 | RAB GTPase activating | 23637 | Hs.271341 | NM_012197 |
| | | protein 1 | | | |
| 205321_at | EIF2S3 | eukaryotic translation | 1968 | Hs.539684 | NM_001415 |
| | | initiation factor 2, | | | |
| | | subunit 3 gamma, | | | |
| | | 52 kDa | | | |
| 206582_s_at | GPR56 | G protein-coupled | 9289 | Hs.513633 | NM_005682 |
| | | receptor 56 | | | |
| 207090_x_at | ZFP30 | zinc finger protein 30 | 22835 | Hs.716719 | NM_014898 |
| | | homolog (mouse) | | | |
| 207836_s_at | RBPMS | RNA binding protein | 11030 | Hs.334587 | NM_006867 |
| | | with multiple splicing | | | |
| 208993_s_at | PPIG | peptidylprolyl | 9360 | Hs.470544 | AW340788 |
| | | isomerase G | | | |
| | | (cyclophilin G) | | | |
| 209272_at | NAB1 | NGFI-A binding protein | 4664 | Hs.570078 | AF045451 |
| | | 1 (EGR1 binding | | | |
| | | protein 1) | | | |
| 209487_at | RBPMS | RNA binding protein | 11030 | Hs.334587 | D84109 |
| | | with multiple splicing | | | |
| 209488_s_at | RBPMS | RNA binding protein | 11030 | Hs.334587 | D84109 |
| | | with multiple splicing | | | |
| 211113_s_at | ABCG1 | ATP-binding cassette, | 9619 | Hs.124649 | U34919 |
| | | sub-family G (WHITE), | | | |
| | | member 1 | | | |
| 212676_at | NF1 | neurofibromin 1 | 4763 | Hs.113577 | AW293356 |
| 212976_at | LRRC8B | leucine rich repeat | 23507 | Hs.482017 | R41498 |
| | | containing 8 family, | | | |
| | | member B | | | |
| 213056_at | FRMD4B | FERM domain | 23150 | Hs.709671 | AU145019 |
| | | containing 4B | | | |
| 214252_s_at | CLN5 | ceroid-lipofuscinosis, | 1203 | Hs.30213 | AV700514 |
| | | neuronal 5 | | | |
| 215411_s_at | TRAF3IP2 | TRAF3 interacting | 10758 | Hs.654708 | AL008730 |
| | | protein 2 | | | |
| 216262_s_at | TGIF2 | TGFB-induced factor | 60436 | Hs.632264 | AL050318 |
| | | homeobox 2 | | | |
| 218183_at | C16orf5 | chromosome 16 open | 29965 | Hs.654653 | NM_013399 |
| | | reading frame 5 | | | |
| 218907_s_at | LRRC61 | leucine rich repeat | 65999 | Hs.647119 | NM_023942 |
| | | containing 61 | | | |
| 219871_at | FLJ13197 | hypothetical FLJ13197 | 79667 | Hs.29725 | NM_024614 |
| 220128_s_at | NIPAL2 | NIPA-like domain | 79815 | Hs.309489 | NM_024759 |
| | | containing 2 | | | |
| 221621_at | C17orf86 | chromosome 17 open | 654434 | — | AF130050 |
| | | reading frame 86 | | | |
| 41113_at | ZNF500 | zinc finger protein 500 | 26048 | Hs.513316 | AI871396 |

TABLE 3
HSC probe set

| | | |
|---|---|---|
| Probe Set ID | probe sequence | Sequence ID No. |

| | | |
|---|---|---|
| 200672_x_at | 5′-AAAGACTGCTGCTTCTGGAATTCCC-3′ | SEQ ID NO: 281 |
| 200672_x_at | 5′-AAGAAGCTGTCTGCGAAGTGGCCCT-3′ | SEQ ID NO: 282 |
| 200672_x_at | 5′-AAGCAGGTCCTGGCACAATGTTTAT-3′ | SEQ ID NO: 283 |
| 200672_x_at | 5′-ACACATGGATCCAGGCTATCTCTTC-3′ | SEQ ID NO: 284 |
| 200672_x_at | 5′-AGAGAAGCGGTTCAGCCTTTTTGGC-3′ | SEQ ID NO: 285 |
| 200672_x_at | 5′-AGCGAGGTCCCTGTGAGTTTGAAAG-3′ | SEQ ID NO: 286 |
| 200672_x_at | 5′-CCTTCTCTTACCTTTTCAGTGAAAT-3′ | SEQ ID NO: 287 |
| 200672_x_at | 5′-CGCCATCTCCTCTGATAAACACGAG-3′ | SEQ ID NO: 288 |
| 200672_x_at | 5′-CTGTGCCTAATGTTCCTCAATGTGG-3′ | SEQ ID NO: 289 |
| 200672_x_at | 5′-GAACCAACACATTACTCTCTGTGCC-3′ | SEQ ID NO: 290 |
| 200672_x_at | 5′-GGCAATGAGTACCTCTTCCAAGCCA-3′ | SEQ ID NO: 291 |
| 201889_at | 5′-AAGCAGTATCTGTTATTTAGCTGTA-3′ | SEQ ID NO: 292 |
| 201889_at | 5′-AATACTTCCCTCAATTCTGTAAATT-3′ | SEQ ID NO: 293 |
| 201889_at | 5′-AATTTAGTGATCAAACTGCCATTCA-3′ | SEQ ID NO: 294 |
| 201889_at | 5′-ATGACTTTATACCCAATTCTACATA-3′ | SEQ ID NO: 295 |
| 201889_at | 5′-GATCTATCTTTTTTTGTTACCTTCA-3′ | SEQ ID NO: 296 |
| 201889_at | 5′-GCCATTCACAGTGTAAGGCAGCACT-3′ | SEQ ID NO: 297 |
| 201889_at | 5′-GGCAGCACTTAAATTTCGAACCTAA-3′ | SEQ ID NO: 298 |
| 201889_at | 5′-TTACCTTCAGATGTTCACTAAATAA-3′ | SEQ ID NO: 299 |
| 201889_at | 5′-TTGACCCCAAATGACTTTATACCCA-3′ | SEQ ID NO: 300 |
| 201889_at | 5′-TTGGGATTTTTGGTGCTTATATGCT-3′ | SEQ ID NO: 301 |
| 201889_at | 5′-TTTGGAGTACTGTTTCTTCCTTCAA-3′ | SEQ ID NO: 302 |
| 202551_s_at | 5′-ACCCATTTGTGCATTGAGTTTTCTT-3′ | SEQ ID NO: 303 |
| 202551_s_at | 5′-AGCACTTTTATACTAATTAACCCAT-3′ | SEQ ID NO: 304 |
| 202551_s_at | 5′-GAGCAGTCAGCATTGCACCTGCTAT-3′ | SEQ ID NO: 305 |
| 202551_s_at | 5′-GATACCCAGTATGCTTAACGTGAAA-3′ | SEQ ID NO: 306 |
| 202551_s_at | 5′-GATGGCAGTTCTTATCTGCATCACT-3′ | SEQ ID NO: 307 |
| 202551_s_at | 5′-GCATTGCACCTGCTATGGAGAAGGG-3′ | SEQ ID NO: 308 |
| 202551_s_at | 5′-GCTCACTGGCCAGAGACATTGATGG-3′ | SEQ ID NO: 309 |
| 202551_s_at | 5′-GGAAGTTTGTTGTAGTATGCCTCAA-3′ | SEQ ID NO: 310 |
| 202551_s_at | 5′-GTAAATACTTGGACAGAGGTTGCTG-3′ | SEQ ID NO: 311 |
| 202551_s_at | 5′-GTTTTCAATTTGCTCACTGGCCAGA-3′ | SEQ ID NO: 312 |
| 202551_s_at | 5′-TGGTAACTTTTCAAACAGCCCTTAG-3′ | SEQ ID NO: 313 |
| 203139_at | 5′-CAGAAGACCCCTGACTCATCATTTG-3′ | SEQ ID NO: 314 |
| 203139_at | 5′-CAGTCCCTTATAATTGGTGCATAGC-3′ | SEQ ID NO: 315 |
| 203139_at | 5′-CATTCCCTCTCATCTCAGGTAGAAG-3′ | SEQ ID NO: 316 |
| 203139_at | 5′-CCTCCTCCAGGGTGATTTTATGATC-3′ | SEQ ID NO: 317 |
| 203139_at | 5′-CTCATCATTTGTGGCAGTCCCTTAT-3′ | SEQ ID NO: 318 |
| 203139_at | 5′-GATCCTGGTTTCATAACTTCCTGTA-3′ | SEQ ID NO: 319 |
| 203139_at | 5′-GATGGTTTCCACATTTAGATCCTGG-3′ | SEQ ID NO: 320 |
| 203139_at | 5′-TACACACTGTCATGCTTCATCATTC-3′ | SEQ ID NO: 321 |
| 203139_at | 5′-TGATCAGTGTTGTTGCTCTAGGAAG-3′ | SEQ ID NO: 322 |
| 203139_at | 5′-TGTCCTAATTCTTCTGTCCTGAGAA-3′ | SEQ ID NO: 323 |
| 203139_at | 5′-TTTTCCGTTTGCTTTTGTTCCAATG-3′ | SEQ ID NO: 324 |
| 204069_at | 5′-AAGCCTTACAGTTATCCTGCAAGGG-3′ | SEQ ID NO: 325 |
| 204069_at | 5′-ATAGTCCCACCTTGGAGCATTTATG-3′ | SEQ ID NO: 326 |
| 204069_at | 5′-ATCAGCTGTTGCAGGCAGTGTCTTA-3′ | SEQ ID NO: 327 |
| 204069_at | 5′-CACCTTATACATCACTTCCTGTTTT-3′ | SEQ ID NO: 328 |
| 204069_at | 5′-CATCAAGCATCATTGTCCCCATGCA-3′ | SEQ ID NO: 329 |
| 204069_at | 5′-CGCCTAGGATTTCAGCCATGCGCGC-3′ | SEQ ID NO: 330 |
| 204069_at | 5′-GAAGCCTAATTGTCACATCAAGCAT-3′ | SEQ ID NO: 331 |
| 204069_at | 5′-GAGCAAAGCATCGGTCATGTGTGTA-3′ | SEQ ID NO: 332 |
| 204069_at | 5′-GCATGTCTAATTCATTTACTCACCA-3′ | SEQ ID NO: 333 |
| 204069_at | 5′-GTGTATTTTTTCATAGTCCCACCTT-3′ | SEQ ID NO: 334 |
| 204069_at | 5′-TCTTTCTTCTCGCCTAGGATTTCAG-3′ | SEQ ID NO: 335 |
| 204304_s_at | 5′-AACCTACAGCATATTCTTCACGCAG-3′ | SEQ ID NO: 336 |
| 204304_s_at | 5′-AAGATTGGCCATGTTCCACTTGGAA-3′ | SEQ ID NO: 337 |
| 204304_s_at | 5′-ACAATTCTTAGATCTGGTGTCCAGC-3′ | SEQ ID NO: 338 |
| 204304_s_at | 5′-ACAGATGCCAATTACGGTGTACAGT-3′ | SEQ ID NO: 339 |
| 204304_s_at | 5′-GAATTCCAGATGTAGGCATTCCCCC-3′ | SEQ ID NO: 340 |
| 204304_s_at | 5′-GAGAAGATCCTGTCACAATTCTTAG-3′ | SEQ ID NO: 341 |
| 204304_s_at | 5′-GAGTGCAGCTAACATGAGTATCATC-3′ | SEQ ID NO: 342 |
| 204304_s_at | 5′-GAGTTTGGTCCCTAAATTTGCATGA-3′ | SEQ ID NO: 343 |
| 204304_s_at | 5′-GCGTAACTCCATCTGACAAATTCAA-3′ | SEQ ID NO: 344 |
| 204304_s_at | 5′-TAGAGAAACCTGCGTAACTCCATCT-3′ | SEQ ID NO: 345 |
| 204304_s_at | 5′-TGCTTCAGGAGTTTCATGTTGGATC-3′ | SEQ ID NO: 346 |
| 204753_s_at | 5′-AGTTCCTGGAATGGCACGTTGCTGC-3′ | SEQ ID NO: 347 |
| 204753_s_at | 5′-ATTTTAAGCCCTATCACTGACACAT-3′ | SEQ ID NO: 348 |
| 204753_s_at | 5′-CACTGACACATCAGCATGTTTTCTG-3′ | SEQ ID NO: 349 |
| 204753_s_at | 5′-CTGCCACAAAAATGTTCACTTCGAA-3′ | SEQ ID NO: 350 |
| 204753_s_at | 5′-GAATGGCACGTTGCTGCCAGTGCCC-3′ | SEQ ID NO: 351 |
| 204753_s_at | 5′-GATGACGAATCCTGCTCTAAAATAC-3′ | SEQ ID NO: 352 |
| 204753_s_at | 5′-GGCCCGCACGTTTTATGAGGTTGAT-3′ | SEQ ID NO: 353 |
| 204753_s_at | 5′-GGCTTGTGATGACGAATCCTGCTCT-3′ | SEQ ID NO: 354 |
| 204753_s_at | 5′-GTCAGTTAACGTCACCCAAAAGCAC-3′ | SEQ ID NO: 355 |
| 204753_s_at | 5′-TATCGGTGCTATGTGTTTGGTTTAT-3′ | SEQ ID NO: 356 |
| 204753_s_at | 5′-TTATGACAGTATCGAGGCTTGTGAT-3′ | SEQ ID NO: 357 |
| 204754_at | 5′-AGTCCAAACCTTTATCTGTCTGTTA-3′ | SEQ ID NO: 358 |
| 204754_at | 5′-CAACACCACAAAGATCGCATCTGTT-3′ | SEQ ID NO: 359 |
| 204754_at | 5′-CAAGGCATGGGACCAGGCCTGCTTG-3′ | SEQ ID NO: 360 |
| 204754_at | 5′-CCACTGGCAAGGCCAAGGTCTCCTC-3′ | SEQ ID NO: 361 |
| 204754_at | 5′-GAGCAAAGCCTTATCCGAATCGGAT-3′ | SEQ ID NO: 362 |
| 204754_at | 5′-GGATTTAGCACTGGGGTCTCAGCAC-3′ | SEQ ID NO: 363 |
| 204754_at | 5′-GGCCTGCTTGCCTATGTGTGATGGC-3′ | SEQ ID NO: 364 |
| 204754_at | 5′-GTCAATTAGAGCGATCCCAAGGCAT-3′ | SEQ ID NO: 365 |
| 204754_at | 5′-GTCTGAGACTAAGTGATCTGCCCTC-3′ | SEQ ID NO: 366 |
| 204754_at | 5′-GTCTTTAATTTTGAGCACCTTACCA-3′ | SEQ ID NO: 367 |
| 204754_at | 5′-TCCTCCACGTTTTTTCTGCAATTAA-3′ | SEQ ID NO: 368 |
| 204755_x_at | 5′-AAGGTGTTCATTTTGTCACAAGCTG-3′ | SEQ ID NO: 369 |
| 204755_x_at | 5′-ATGAGCATCTCAAATGTTTTCTGCA-3′ | SEQ ID NO: 370 |
| 204755_x_at | 5′-ATGGCCGTATCAAATGGTAGCTGAA-3′ | SEQ ID NO: 371 |
| 204755_x_at | 5′-ATGGGATTTTCTAGTTTCCTGCCTT-3′ | SEQ ID NO: 372 |
| 204755_x_at | 5′-ATTTGAGCACTGGTCTCTCTTGGAA-3′ | SEQ ID NO: 373 |
| 204755_x_at | 5′-CTCGTCAATCCATCAGCAATGCTTC-3′ | SEQ ID NO: 374 |
| 204755_x_at | 5′-GCAATGCTTCTCTCATAGTGTCATA-3′ | SEQ ID NO: 375 |
| 204755_x_at | 5′-GGACCATCCAAATTTATGGCCGTAT-3′ | SEQ ID NO: 376 |
| 204755_x_at | 5′-GGACGTAGAGTTGGCCTTTTTACAG-3′ | SEQ ID NO: 377 |
| 204755_x_at | 5′-TCCTGCCTTCAGAGTATCTAATCCT-3′ | SEQ ID NO: 378 |
| 204755_x_at | 5′-TTAATGATCTGGTGGTCTCCTCGTC-3′ | SEQ ID NO: 379 |
| 204917_s_at | 5′-ATGCATATTCAACACACTGCCTTAT-3′ | SEQ ID NO: 380 |
| 204917_s_at | 5′-CCAAGTCCTTTAACTCGTTGCAGTC-3′ | SEQ ID NO: 381 |
| 204917_s_at | 5′-CCAGTCCTTGGCTGTATCCATGTAA-3′ | SEQ ID NO: 382 |
| 204917_s_at | 5′-GAAATCCCCGGGAAGAGTTAGCCTG-3′ | SEQ ID NO: 383 |
| 204917_s_at | 5′-GAATTGCTGTCTAGCCTTAGTCAAT-3′ | SEQ ID NO: 384 |
| 204917_s_at | 5′-GAGTTAGCCTGGATAGCCTTGAAAA-3′ | SEQ ID NO: 385 |
| 204917_s_at | 5′-GTATCATGTATCTCTCTGTGGTGGT-3′ | SEQ ID NO: 386 |
| 204917_s_at | 5′-GTGGTGGTTCATTCCACAGGACGAA-3′ | SEQ ID NO: 387 |
| 204917_s_at | 5′-TAAGTACTTGGTCCCGTGGATGCTC-3′ | SEQ ID NO: 388 |
| 204917_s_at | 5′-TGAAAGTTGGGGCCCAGTCCTTGGC-3′ | SEQ ID NO: 389 |
| 204917_s_at | 5′-TGGATGCTCTTTCAATGCAGCACCC-3′ | SEQ ID NO: 390 |
| 205376_at | 5′-AAATCTCCTTCAAAATATCCAATCC-3′ | SEQ ID NO: 391 |
| 205376_at | 5′-AAGCTGACACCTAAGTTTACCAACA-3′ | SEQ ID NO: 392 |
| 205376_at | 5′-ACATGCTACAGCTGATGGCTTTCCC-3′ | SEQ ID NO: 393 |
| 205376_at | 5′-CAAGGACTTCTTTATCCGAGCGCTG-3′ | SEQ ID NO: 394 |
| 205376_at | 5′-CCGAGCGCTGGATTGCATGAGAAGA-3′ | SEQ ID NO: 395 |
| 205376_at | 5′-CTGGCTGCAACGATTTGCCGCAAAC-3′ | SEQ ID NO: 396 |
| 205376_at | 5′-GAATGGTATTCGTTTCACCTGTTGT-3′ | SEQ ID NO: 397 |
| 205376_at | 5′-GATGAGCACCAGTTACACAAGGACT-3′ | SEQ ID NO: 398 |
| 205376_at | 5′-GATGCCTCCTGATTATATTTCACAT-3′ | SEQ ID NO: 399 |
| 205376_at | 5′-GTAGAAATTATGTGGCTGGCTGCAA-3′ | SEQ ID NO: 400 |
| 205376_at | 5′-GTCAGTGACACTTGAACAATGCTCA-3′ | SEQ ID NO: 401 |
| 205984_at | 5′-AAATATCTGATCTTACCCTGGGACA-3′ | SEQ ID NO: 402 |
| 205984_at | 5′-AGATGACGCCTTTAGCTGATCTCTG-3′ | SEQ ID NO: 403 |
| 205984_at | 5′-ATCGTCAGCTGGAGCCGTACGAGCT-3′ | SEQ ID NO: 404 |
| 205984_at | 5′-GAAACTGCAGCTTCTCCATAATTTA-3′ | SEQ ID NO: 405 |
| 205984_at | 5′-GAGGGAACTGGATTGGACCCTTCCA-3′ | SEQ ID NO: 406 |
| 205984_at | 5′-GCTGTGACAACACTGTGGTGCGCAT-3′ | SEQ ID NO: 407 |
| 205984_at | 5′-GGAATTCTGTTTGTCTGGTCTTTGA-3′ | SEQ ID NO: 408 |
| 205984_at | 5′-GTGCGCATGGTCTCCAGTGGAAAAC-3′ | SEQ ID NO: 409 |
| 205984_at | 5′-TAACCAACCCAGTGATTTACATGCT-3′ | SEQ ID NO: 410 |
| 205984_at | 5′-TCATACCAGTCAGTATTTCCCAGCC-3′ | SEQ ID NO: 411 |
| 205984_at | 5′-TTCATGGCCCGGCCCAGATGAAAGT-3′ | SEQ ID NO: 412 |
| 206385_s_at | 5′-AAAGCCCTTCATCTAATATTTGTTG-3′ | SEQ ID NO: 413 |
| 206385_s_at | 5′-AAATGCTTGCCGCTTTAGAGGTGGA-3′ | SEQ ID NO: 414 |
| 206385_s_at | 5′-AAGCCAATCATTTGTAACCATTCTA-3′ | SEQ ID NO: 415 |
| 206385_s_at | 5′-ACCATACACTGGATGACCTAGTCGA-3′ | SEQ ID NO: 416 |
| 206385_s_at | 5′-GCATTCATTGACACATAGCTCTAAT-3′ | SEQ ID NO: 417 |
| 206385_s_at | 5′-GCTAGTAGAATGGCAGCACGCTGTA-3′ | SEQ ID NO: 418 |
| 206385_s_at | 5′-GTAACCATTCTAGCAGTGTCATATT-3′ | SEQ ID NO: 419 |
| 206385_s_at | 5′-GTAGACACCTTTCAGTAAGCCAATC-3′ | SEQ ID NO: 420 |
| 206385_s_at | 5′-TATACGGTAGTTGCTTTAGGGGGTG-3′ | SEQ ID NO: 421 |
| 206385_s_at | 5′-TGGTGCTCATAAAAGGCCCCAGTCG-3′ | SEQ ID NO: 422 |
| 206385_s_at | 5′-TTACTGTATTGTGTACTGGCTATAA-3′ | SEQ ID NO: 423 |
| 206478_at | 5′-ACACACTCTTACTCCCGTGATGTGT-3′ | SEQ ID NO: 424 |
| 206478_at | 5′-AGTCAAAGGCTGATGTCCTGTTTCT-3′ | SEQ ID NO: 425 |
| 206478_at | 5′-ATTTGACCACGTCCATTGTTTCCAT-3′ | SEQ ID NO: 426 |
| 206478_at | 5′-CAAGCCATGGCAATATCTGTCCCAC-3′ | SEQ ID NO: 427 |
| 206478_at | 5′-CATCTACATCCATATCATGCCCATG-3′ | SEQ ID NO: 428 |
| 206478_at | 5′-CATGCCCATGCATCTGTAACTTGCT-3′ | SEQ ID NO: 429 |
| 206478_at | 5′-GAGTTTGTTCAATGCATGTGTCTGT-3′ | SEQ ID NO: 430 |
| 206478_at | 5′-GTGATGTGTGTTAAGGGCTCCGATG-3′ | SEQ ID NO: 431 |
| 206478_at | 5′-TGAATTTCTGCACGCTGTTGTCTGT-3′ | SEQ ID NO: 432 |
| 206478_at | 5′-TGTAACTTGCTTTTCCCGTGTAAGA-3′ | SEQ ID NO: 433 |
| 206478_at | 5′-TGTTTCCATCTTTTGGGCTGTTCTT-3′ | SEQ ID NO: 434 |
| 206683_at | 5′-AAAACCTTCCGAGTGAGCTCACATC-3′ | SEQ ID NO: 435 |
| 206683_at | 5′-AACATGCAGCAGTTTTCAGTGGAGA-3′ | SEQ ID NO: 436 |
| 206683_at | 5′-ACATCTTATTCGACACTTTAGAATT-3′ | SEQ ID NO: 437 |
| 206683_at | 5′-AGAGCTCAAACCTTAGTCAACACCA-3′ | SEQ ID NO: 438 |
| 206683_at | 5′-AGCTCAAAACTTGCTAGGCATCAGA-3′ | SEQ ID NO: 439 |
| 206683_at | 5′-GAACTCACATCTTATCAGGCATCAG-3′ | SEQ ID NO: 440 |
| 206683_at | 5′-GCTCAGATCTTACTAGACATCGGCG-3′ | SEQ ID NO: 441 |
| 206683_at | 5′-GCTTTCAGGCACAGCTCAAAACTTG-3′ | SEQ ID NO: 442 |
| 206683_at | 5′-GCTTTGCAGAGAGCTCAGATCTTAC-3′ | SEQ ID NO: 443 |
| 206683_at | 5′-GGAGAGCATTCAACCTGAACTCACA-3′ | SEQ ID NO: 444 |
| 206683_at | 5′-TAGACATCGGCGAATTCACACTGGG-3′ | SEQ ID NO: 445 |
| 208892_s_at | 5′-ATGGCGAAGTCTTTAGTCTTTTTCA-3′ | SEQ ID NO: 446 |
| 208892_s_at | 5′-ATTTGCAGCATGCTTGACTTTACCA-3′ | SEQ ID NO: 447 |
| 208892_s_at | 5′-CACTAAGACCTTGTTATGGCGAAGT-3′ | SEQ ID NO: 448 |
| 208892_s_at | 5′-CGGACACTATTATCACTAAGACCTT-3′ | SEQ ID NO: 449 |
| 208892_s_at | 5′-GACTTTACCAATTCTGATGACATCT-3′ | SEQ ID NO: 450 |
| 208892_s_at | 5′-GATAATCTGGGAAAGACACCAAATC-3′ | SEQ ID NO: 451 |
| 208892_s_at | 5′-GATGACATCTTTACGGACACTATTA-3′ | SEQ ID NO: 452 |
| 208892_s_at | 5′-GTTGTCGCAAAGGGGATAATCTGGG-3′ | SEQ ID NO: 453 |
| 208892_s_at | 5′-TATGCCTTACCTTTGTAAATATTTT-3′ | SEQ ID NO: 454 |
| 208892_s_at | 5′-TGCTTGTGTTGTCGCAAAGGGGATA-3′ | SEQ ID NO: 455 |
| 208892_s_at | 5′-TTAGTCTTTTTCATGTATTTTCCTC-3′ | SEQ ID NO: 456 |
| 209487_at | 5′-AACTATTTCTTGGCGACCTTTGAGA-3′ | SEQ ID NO: 457 |
| 209487_at | 5′-AATTAGATTTGTCTCTGGGAATGTG-3′ | SEQ ID NO: 458 |
| 209487_at | 5′-CTTTCACCAAAACTATTTCTTGGCG-3′ | SEQ ID NO: 459 |
| 209487_at | 5′-GGAGCTCCCATGTTGAATTTGTTTG-3′ | SEQ ID NO: 460 |
| 209487_at | 5′-GTGTTTCTCTCCTGAGGCAAAGCCC-3′ | SEQ ID NO: 461 |
| 209487_at | 5′-GTGTTTGTAACATACCAACCTACTG-3′ | SEQ ID NO: 462 |
| 209487_at | 5′-TCTTGGCGACCTTTGAGAGATTTCA-3′ | SEQ ID NO: 463 |
| 209487_at | 5′-TGTAACATACCAACCTACTGCAGAC-3′ | SEQ ID NO: 464 |
| 209487_at | 5′-TTGTCCACTTCTCCAGCAAATTAGA-3′ | SEQ ID NO: 465 |
| 209487_at | 5′-TTGTCTCTGGGAATGTGTTTGTAAC-3′ | SEQ ID NO: 466 |
| 209487_at | 5′-TTTTGTCCACTTCTCCAGCAAATTA-3′ | SEQ ID NO: 467 |
| 209560_s_at | 5′-AATCTGGTGAACGCTACGCTTACAT-3′ | SEQ ID NO: 468 |
| 209560_s_at | 5′-CAAGTGCGAGACCTGGGTGTCCAAC-3′ | SEQ ID NO: 469 |
| 209560_s_at | 5′-GAGGAGATCTAAGCAGCGTTCCCAC-3′ | SEQ ID NO: 470 |
| 209560_s_at | 5′-GAGTTCCGCAGAGCTTACTATACGC-3′ | SEQ ID NO: 471 |
| 209560_s_at | 5′-GTATCGTCTTCCTCAACAAGTGCGA-3′ | SEQ ID NO: 472 |
| 209560_s_at | 5′-GTTCGCTATCTCTTGTGTCAAATCT-3′ | SEQ ID NO: 473 |
| 209560_s_at | 5′-TACTATACGCGGTCTGTCCTAATCT-3′ | SEQ ID NO: 474 |
| 209560_s_at | 5′-TCGACATGACCACCTTCAGCAAGGA-3′ | SEQ ID NO: 475 |
| 209560_s_at | 5′-TGCAAAAACAATCCTCTTTCTCTCT-3′ | SEQ ID NO: 476 |
| 209560_s_at | 5′-TGCGCTACAACCACATGCTGCGGAA-3′ | SEQ ID NO: 477 |
| 209560_s_at | 5′-TGTCCTAATCTTTGTGGTGTTCGCT-3′ | SEQ ID NO: 478 |
| 209993_at | 5′-AAAGCGCCAGTGAACTCTGACTGTA-3′ | SEQ ID NO: 479 |
| 209993_at | 5′-AACAACGCATTGCCATAGCTCGTGC-3′ | SEQ ID NO: 480 |
| 209993_at | 5′-AGCCACGTCAGCTCTGGATACAGAA-3′ | SEQ ID NO: 481 |
| 209993_at | 5′-CAAAGGAACTCAGCTCTCTGGTGGC-3′ | SEQ ID NO: 482 |
| 209993_at | 5′-CAGCCTCATATTTTGCTTTTGGATG-3′ | SEQ ID NO: 483 |
| 209993_at | 5′-GAGTGAGAGACATCATCAAGTGGAG-3′ | SEQ ID NO: 484 |
| 209993_at | 5′-GCCCTTGTTAGACAGCCTCATATTT-3′ | SEQ ID NO: 485 |
| 209993_at | 5′-GTCACTGCCTAATAAATATAGCACT-3′ | SEQ ID NO: 486 |
| 209993_at | 5′-TCCTCAGTCAAGTTCAGAGTCTTCA-3′ | SEQ ID NO: 487 |
| 209993_at | 5′-TCTGTTTAACATTTCCTCAGTCAAG-3′ | SEQ ID NO: 488 |
| 209993_at | 5′-TTTGGATGAAGCCACGTCAGCTCTG-3′ | SEQ ID NO: 489 |
| 211597_s_at | 5′-AAGCTATGTGTATCTTCTGTGTAAA-3′ | SEQ ID NO: 490 |
| 211597_s_at | 5′-AATGGTGTGGCTAGCATTTCCCTTT-3′ | SEQ ID NO: 491 |
| 211597_s_at | 5′-ACTTCCTTGGAATATAGCTGCATTA-3′ | SEQ ID NO: 492 |
| 211597_s_at | 5′-AGTCACTTTCCTTATGTATCATCTA-3′ | SEQ ID NO: 493 |
| 211597_s_at | 5′-CTTCCCTAAGTCACTTTCCTTATGT-3′ | SEQ ID NO: 494 |
| 211597_s_at | 5′-GAAGCCTGTTGGGCCAGAAGACAGA-3′ | SEQ ID NO: 495 |
| 211597_s_at | 5′-GAAGGGAACACATTTCCTTCTGAAC-3′ | SEQ ID NO: 496 |
| 211597_s_at | 5′-GCAATCCAGGCCTCTGTTGAAAAGA-3′ | SEQ ID NO: 497 |
| 211597_s_at | 5′-TAAGTTTGCTTTTGACCATCACCTC-3′ | SEQ ID NO: 498 |
| 211597_s_at | 5′-TAATCCATTTAGCAATCCAGGCCTC-3′ | SEQ ID NO: 499 |
| 211597_s_at | 5′-TCACCTCCCAGTAGCAATTTGCTTT-3′ | SEQ ID NO: 500 |
| 212071_s_at | 5′-AAACCATTTGTATCTGGCATCACTT-3′ | SEQ ID NO: 501 |
| 212071_s_at | 5′-AATTTTCATCTTACTGCACAATCAA-3′ | SEQ ID NO: 502 |
| 212071_s_at | 5′-ACATGCGGCTTTTCTGCATCAACTG-3′ | SEQ ID NO: 503 |
| 212071_s_at | 5′-GAGGCTGGGCCTGAACAGGGAGGTG-3′ | SEQ ID NO: 504 |
| 212071_s_at | 5′-GTGCTCAGTCGTACGACCTGTACCT-3′ | SEQ ID NO: 505 |
| 212071_s_at | 5′-TAACACACGACATGCGGCTTTTCTG-3′ | SEQ ID NO: 506 |
| 212071_s_at | 5′-TAATTTGCTTCATTTCCTTGCTATT-3′ | SEQ ID NO: 507 |
| 212071_s_at | 5′-TAGGAATGAACTCCAGAGGCTGGGC-3′ | SEQ ID NO: 508 |
| 212071_s_at | 5′-TCTAATGGTTACTTGCTCGTGCGTT-3′ | SEQ ID NO: 509 |
| 212071_s_at | 5′-TCTGGCATCACTTACTAACACACGA-3′ | SEQ ID NO: 510 |
| 212071_s_at | 5′-TGCATTTCTCTGTCACTGTAACTAT-3′ | SEQ ID NO: 511 |
| 212488_at | 5′-AAAAGCCATAGCCGAGGACTGTCCC-3′ | SEQ ID NO: 512 |
| 212488_at | 5′-AACACCGCCAGCGTGGATTTTCCAA-3′ | SEQ ID NO: 513 |
| 212488_at | 5′-ACCACCAGAATGCAGTTCCAGCTTA-3′ | SEQ ID NO: 514 |
| 212488_at | 5′-CAGACCACTCTAGCCACAGTATATT-3′ | SEQ ID NO: 515 |
| 212488_at | 5′-CCGTGGACTGCGTCTAGGTCATGTG-3′ | SEQ ID NO: 516 |
| 212488_at | 5′-CTCTGTGGTCCCTTCAAAGTTGTTA-3′ | SEQ ID NO: 517 |
| 212488_at | 5′-GAAAGGCGATCTCTTCACTGTGAAA-3′ | SEQ ID NO: 518 |
| 212488_at | 5′-GAGAGTCTCTGGAGCCCAGGATGCC-3′ | SEQ ID NO: 519 |
| 212488_at | 5′-GGATGCCAGCATGTGCCAATGACTG-3′ | SEQ ID NO: 520 |
| 212488_at | 5′-TGCCAATGACTGTCACCTTCATCTC-3′ | SEQ ID NO: 521 |
| 212488_at | 5′-TGGAAAGTAAGTCTCGCTCTTGCCA-3′ | SEQ ID NO: 522 |
| 212750_at | 5′-AAAATCTTCGCAGATCTTTGATATC-3′ | SEQ ID NO: 523 |
| 212750_at | 5′-AAGGCCTGTGACAGAATTCGCTGTT-3′ | SEQ ID NO: 524 |
| 212750_at | 5′-ATGGGCATTGCAAGTGCCACCGTGC-3′ | SEQ ID NO: 525 |
| 212750_at | 5′-CCTGCTTCCCATGGGCATTGCAAGT-3′ | SEQ ID NO: 526 |
| 212750_at | 5′-CTCCCCAACAGGTCTCTCTTGTTGG-3′ | SEQ ID NO: 527 |
| 212750_at | 5′-CTCCGCAATAATTCACCAGACCAGA-3′ | SEQ ID NO: 528 |
| 212750_at | 5′-CTGCCCCAGGGCACATAAGAGCAAA-3′ | SEQ ID NO: 529 |
| 212750_at | 5′-GGATGACTCTGCAAAAGTGACCCCC-3′ | SEQ ID NO: 530 |
| 212750_at | 5′-GTATACTGTATCAGCAGCTTTGTGT-3′ | SEQ ID NO: 531 |
| 212750_at | 5′-TAACTTGGGGATGGTCTCCCCTGCC-3′ | SEQ ID NO: 532 |
| 212750_at | 5′-TACTGAGGTAACTTCCACGTAGCCC-3′ | SEQ ID NO: 533 |
| 213094_at | 5′-AATTCAGACTCTCTTTTCATTATGT-3′ | SEQ ID NO: 534 |
| 213094_at | 5′-AGAGTCATAGTCTAGGATCCTGAGA-3′ | SEQ ID NO: 535 |
| 213094_at | 5′-GATTGAGCCAAATTCTGTTGTCAGT-3′ | SEQ ID NO: 536 |
| 213094_at | 5′-GTTCTAAGCATGCAGTTCTCACCTC-3′ | SEQ ID NO: 537 |
| 213094_at | 5′-TAGCTAATTTGCCATTTTACTTAAA-3′ | SEQ ID NO: 538 |
| 213094_at | 5′-TAGCTGGGGAGCCTAAATTTAGTTC-3′ | SEQ ID NO: 539 |
| 213094_at | 5′-TCCTTTCTTAGCTTGATATTGCCTA-3′ | SEQ ID NO: 540 |
| 213094_at | 5′-TGTCACCATTCACTTGCATTGTAAA-3′ | SEQ ID NO: 541 |
| 213094_at | 5′-TTCTGTTGTCAGTTCTAAGCATGCA-3′ | SEQ ID NO: 542 |
| 213094_at | 5′-TTGATATTGCCTAGCTTTGTTGTTT-3′ | SEQ ID NO: 543 |
| 213094_at | 5′-TTTTCTTTGTCTGTTGTTGGCATAG-3′ | SEQ ID NO: 544 |
| 213510_x_at | 5′-AATATCTAGTTCTCAGAGCATTTGG-3′ | SEQ ID NO: 545 |
| 213510_x_at | 5′-ACTTGTTGACAATGCACTGACTTTA-3′ | SEQ ID NO: 546 |
| 213510_x_at | 5′-ATATAAAATCTGTCCTTTCCTACCT-3′ | SEQ ID NO: 547 |
| 213510_x_at | 5′-CTACTAATGTTGTTTGATCTGTGTT-3′ | SEQ ID NO: 548 |
| 213510_x_at | 5′-GATCTGTGTTTGTTATACTGGTTGT-3′ | SEQ ID NO: 549 |
| 213510_x_at | 5′-GGAGTGGCCTAAATTATCTAATGTA-3′ | SEQ ID NO: 550 |
| 213510_x_at | 5′-GGTTATCTTAAATGGCTACCTAAAT-3′ | SEQ ID NO: 551 |
| 213510_x_at | 5′-TAACCACATTCACCTTGTAAATGAC-3′ | SEQ ID NO: 552 |
| 213510_x_at | 5′-TGGCTACCTAAATTGAAATCCTTTT-3′ | SEQ ID NO: 553 |
| 213510_x_at | 5′-TTTATCTGTAACTGTTATCCAAACA-3′ | SEQ ID NO: 554 |
| 213510_x_at | 5′-TTTCCTACCTGGACATGTCCCATTA-3′ | SEQ ID NO: 555 |
| 213844_at | 5′-AAATAGCACATGCTCTTTGCCTCTC-3′ | SEQ ID NO: 556 |
| 213844_at | 5′-AGGTGACTTTCTGAAACTCCCTTGT-3′ | SEQ ID NO: 557 |
| 213844_at | 5′-AGTAGATCTGCTTTCTGTTCATCTC-3′ | SEQ ID NO: 558 |
| 213844_at | 5′-CCCTGGATGCGCAAGCTGCACATAA-3′ | SEQ ID NO: 559 |
| 213844_at | 5′-CGTCCCTGAGTATCTGAGCGTTTAA-3′ | SEQ ID NO: 560 |
| 213844_at | 5′-CGTTACCTGACCCGCAGAAGGAGGA-3′ | SEQ ID NO: 561 |
| 213844_at | 5′-GTTCATCTCTTTGTCCTGAATGGCT-3′ | SEQ ID NO: 562 |
| 213844_at | 5′-GTTTATTGCCATTATAGCGCCTGTA-3′ | SEQ ID NO: 563 |
| 213844_at | 5′-TAGCGGATCCCGCGTAGTGTCAGTA-3′ | SEQ ID NO: 564 |
| 213844_at | 5′-TCATGACAACATAGGCGGCCCGGAA-3′ | SEQ ID NO: 565 |
| 213844_at | 5′-TCGTTGCCCTAATTCATCTTTTAAT-3′ | SEQ ID NO: 566 |
| 218379_at | 5′-AGCATAAATCCCCTTTTCAGGAAGA-3′ | SEQ ID NO: 567 |
| 218379_at | 5′-AGCCTTTAAGTGCTGCTTCTGTCAG-3′ | SEQ ID NO: 568 |
| 218379_at | 5′-ATCCCATTTGAGGTATAAGTCACTC-3′ | SEQ ID NO: 569 |
| 218379_at | 5′-CAGTGTTAGCATAAATCCCCTTTTC-3′ | SEQ ID NO: 570 |
| 218379_at | 5′-CCACAGCATTTGTACTGTTCCTTTT-3′ | SEQ ID NO: 571 |
| 218379_at | 5′-GAGCTTTACCCTAGTTGAACATACA-3′ | SEQ ID NO: 572 |
| 218379_at | 5′-GATTTACACATACTGTTTCATTCTA-3′ | SEQ ID NO: 573 |
| 218379_at | 5′-GGAAGTTAAAATATCTCTACACGTA-3′ | SEQ ID NO: 574 |
| 218379_at | 5′-GTGACATGCTCTTGAGCTTTACCCT-3′ | SEQ ID NO: 575 |
| 218379_at | 5′-GTGCTGCTTCTGTCAGTCAAACGTT-3′ | SEQ ID NO: 576 |
| 218379_at | 5′-TTCAAAGTGCCCAGACTGTGTACAA-3′ | SEQ ID NO: 577 |
| 218723_s_at | 5′-ACTGAATTCTCCAACAGACTCTACC-3′ | SEQ ID NO: 578 |
| 218723_s_at | 5′-CAGGCTCACCTTAAAATCAGCCCTT-3′ | SEQ ID NO: 579 |
| 218723_s_at | 5′-CCACTGTCACTCCTCAGAAAGCTAA-3′ | SEQ ID NO: 580 |
| 218723_s_at | 5′-GAACAGACGATCCATGCTAATATTG-3′ | SEQ ID NO: 581 |
| 218723_s_at | 5′-GAAGCCTTCATTGCTGATCTTGACA-3′ | SEQ ID NO: 582 |
| 218723_s_at | 5′-GAGGACCTGCTAAAATCAGCTACTA-3′ | SEQ ID NO: 583 |
| 218723_s_at | 5′-GCTTCAGAAAGTTCCGAGGACCTGC-3′ | SEQ ID NO: 584 |
| 218723_s_at | 5′-GGACAAAGACGTGCACTCAACCTTC-3′ | SEQ ID NO: 585 |
| 218723_s_at | 5′-TAGCAGTAAGCTTTCCCATTATAAT-3′ | SEQ ID NO: 586 |
| 218723_s_at | 5′-TCAGCTACTAGAATCTGCTGCCAGA-3′ | SEQ ID NO: 587 |
| 218723_s_at | 5′-TCTGGGTCCTTTCATCATAAGGGAG-3′ | SEQ ID NO: 588 |
| 218899_s_at | 5′-AATGCATCTGGCTACTTTTTCATGT-3′ | SEQ ID NO: 589 |
| 218899_s_at | 5′-ACAAGACTTTACCATACACGCAACT-3′ | SEQ ID NO: 590 |
| 218899_s_at | 5′-ACTGGCATTACTCAGCAGGAGCCCC-3′ | SEQ ID NO: 591 |
| 218899_s_at | 5′-AGAAACTAATCCTTACTATCCTATT-3′ | SEQ ID NO: 592 |
| 218899_s_at | 5′-ATTAGGATACCACTTTTCATTGCAA-3′ | SEQ ID NO: 593 |
| 218899_s_at | 5′-CAAGTTCAAGGGCTCTTTCTCCCTG-3′ | SEQ ID NO: 594 |
| 218899_s_at | 5′-CTGCATCAGTTCACTGCTGCATGTT-3′ | SEQ ID NO: 595 |
| 218899_s_at | 5′-GAAACACTTTCTCACTTACAGGGGA-3′ | SEQ ID NO: 596 |
| 218899_s_at | 5′-GGATTTCACGGAGACAGCAACCAGA-3′ | SEQ ID NO: 597 |
| 218899_s_at | 5′-TGGCTTCTCTTTACAGCTTTGTTTC-3′ | SEQ ID NO: 598 |
| 218899_s_at | 5′-TTCATATGTCCCCACTGGCATTACT-3′ | SEQ ID NO: 599 |
| 218966_at | 5′-AAGAATCCCAATTGCACCTTCTGTT-3′ | SEQ ID NO: 600 |
| 218966_at | 5′-ACTTTCGCTCTCTAATCAGCATTTC-3′ | SEQ ID NO: 601 |
| 218966_at | 5′-ATTGTGTCGGACCCTACTTTTGAGA-3′ | SEQ ID NO: 602 |
| 218966_at | 5′-GCAACCTAAATTACTTTCGCTCTCT-3′ | SEQ ID NO: 603 |
| 218966_at | 5′-GCACCTTCTGTTTCTGACAGTCACA-3′ | SEQ ID NO: 604 |
| 218966_at | 5′-GCATCACCCTGCTAATACATAATAA-3′ | SEQ ID NO: 605 |
| 218966_at | 5′-TAGTCTCTGGCCTGTGGATCCAGTG-3′ | SEQ ID NO: 606 |
| 218966_at | 5′-TCTTACCTGCCAACATATTCACCAT-3′ | SEQ ID NO: 607 |
| 218966_at | 5′-TGGATCCAGTGCTATTCTGTCACCA-3′ | SEQ ID NO: 608 |
| 218966_at | 5′-TGGGAACTGGCTATTCCTTGTCCCG-3′ | SEQ ID NO: 609 |
| 218966_at | 5′-TTGATAAGCACTCCTAGTCTCTGGC-3′ | SEQ ID NO: 610 |
| 219497_s_at | 5′-ATGGTGCTTTATATTTAGATTGGAA-3′ | SEQ ID NO: 611 |
| 219497_s_at | 5′-ATTATTGCTTATGTGCCCTGTTCAA-3′ | SEQ ID NO: 612 |
| 219497_s_at | 5′-ATTCCAGCATCTTACCTTCATATGC-3′ | SEQ ID NO: 613 |
| 219497_s_at | 5′-GAAAGCCCGCTTTAGTCAATACTTT-3′ | SEQ ID NO: 614 |
| 219497_s_at | 5′-GAAAGCTGTTTGTCGTAACTTGAAA-3′ | SEQ ID NO: 615 |
| 219497_s_at | 5′-GGCAGTTGTCTGCATTAACCTGTTC-3′ | SEQ ID NO: 616 |
| 219497_s_at | 5′-GGCCTTTTCTATTCCTGTAATGAAA-3′ | SEQ ID NO: 617 |
| 219497_s_at | 5′-TATCTTTTACTATGGGAGTCACTAT-3′ | SEQ ID NO: 618 |
| 219497_s_at | 5′-TATGTAGTGTGCTTTTTGTCCCTTT-3′ | SEQ ID NO: 619 |
| 219497_s_at | 5′-TATTTGTTTCTGGTCTTTGTTAAGT-3′ | SEQ ID NO: 620 |
| 219497_s_at | 5′-TGTTATTGGCCTTTTCTATTCCTGT-3′ | SEQ ID NO: 621 |
| 220416_at | 5′-AAACCTCAGTTCTGTCACTTCTTAC-3′ | SEQ ID NO: 622 |
| 220416_at | 5′-AAGTGATTCGGGCATATTTGTGTGA-3′ | SEQ ID NO: 623 |
| 220416_at | 5′-AGCTCAAATTTCAGTCCACATATGA-3′ | SEQ ID NO: 624 |
| 220416_at | 5′-CAATGGTTTTTCTAACAACCTCAGT-3′ | SEQ ID NO: 625 |
| 220416_at | 5′-CATCATCCAGACCATTAATAGAATC-3′ | SEQ ID NO: 626 |
| 220416_at | 5′-GAAATGTGAGAGAGGCTCGCCACTA-3′ | SEQ ID NO: 627 |
| 220416_at | 5′-GAGGCTCGCCACTAAGTATTCTAAA-3′ | SEQ ID NO: 628 |
| 220416_at | 5′-GATACTCAGCTGTCATGTTTATAAT-3′ | SEQ ID NO: 629 |
| 220416_at | 5′-GCTCTCAGTCTGTGTCATGTAAGGA-3′ | SEQ ID NO: 630 |
| 220416_at | 5′-TAGTTGCTTTTGATACTCAGCTGTC-3′ | SEQ ID NO: 631 |
| 220416_at | 5′-TTCAAAAAGCTCTCAGTCTGTGTCA-3′ | SEQ ID NO: 632 |
| 221841_s_at | 5′-AAACTGCTGCATACTTTGACAAGGA-3′ | SEQ ID NO: 633 |
| 221841_s_at | 5′-AAAGATCACCTTGTATTCTCTTTAC-3′ | SEQ ID NO: 634 |
| 221841_s_at | 5′-AATCTATATTTGTCTTCCGATCAAC-3′ | SEQ ID NO: 635 |
| 221841_s_at | 5′-ATACCTGGTTTACTTCTTTAGCATT-3′ | SEQ ID NO: 636 |
| 221841_s_at | 5′-ATCCGACTTGAATATTCCTGGACTT-3′ | SEQ ID NO: 637 |
| 221841_s_at | 5′-CAGACAGTCTGTTATGCACTGTGGT-3′ | SEQ ID NO: 638 |
| 221841_s_at | 5′-GATGGTGCTTGGTGAGTCTTGGTTC-3′ | SEQ ID NO: 639 |
| 221841_s_at | 5′-GCCAAGGGGGTGACTGGAAGTTGTG-3′ | SEQ ID NO: 640 |
| 221841_s_at | 5′-GGAAGACCAGAATTCCCTTGAATTG-3′ | SEQ ID NO: 641 |
| 221841_s_at | 5′-GGTTTATTCCCAAGTATGCCTTAAG-3′ | SEQ ID NO: 642 |
| 221841_s_at | 5′-TTTTCTATATAGTTCCTTGCCTTAA-3′ | SEQ ID NO: 643 |
| 222164_at | 5′-AGAAAACACCTGTGAAGCTGGAGGT-3′ | SEQ ID NO: 644 |
| 222164_at | 5′-AGTTGACTTCCATCAGTGTTGAGCC-3′ | SEQ ID NO: 645 |
| 222164_at | 5′-ATAAGAAAATCTCCTTGTGGTGAAG-3′ | SEQ ID NO: 646 |
| 222164_at | 5′-CACTCATCGCTGTTCCGAACAAGTC-3′ | SEQ ID NO: 647 |
| 222164_at | 5′-GAATGTCTAAGTGAAGGGACCAGTT-3′ | SEQ ID NO: 648 |
| 222164_at | 5′-GAGATTGTTAAGCAGTTGACTTCCA-3′ | SEQ ID NO: 649 |
| 222164_at | 5′-GGTGTGTGCTGACTGGATTCAGAGG-3′ | SEQ ID NO: 650 |
| 222164_at | 5′-GGTTCAGAGACATGGGATCGTTTCC-3′ | SEQ ID NO: 651 |
| 222164_at | 5′-TCCATCAGTGTTGAGCCAGGAATTG-3′ | SEQ ID NO: 652 |
| 222164_at | 5′-TCGCTGTTCCGAACAAGTCAGCCAG-3′ | SEQ ID NO: 653 |
| 222164_at | 5′-TGAAGCTGGAGGTGACCATTCACCA-3′ | SEQ ID NO: 654 |
| 226206_at | 5′-AAACAGATCACATGTGGGCCCGTGT-3′ | SEQ ID NO: 655 |
| 226206_at | 5′-AAGAGATCCAGGTCTTTGCGTTTCC-3′ | SEQ ID NO: 656 |
| 226206_at | 5′-AAGCACGGTGTGTTCTGCTTTTCTT-3′ | SEQ ID NO: 657 |
| 226206_at | 5′-AGACGAGGGACTCTTTGTCACGTGG-3′ | SEQ ID NO: 658 |
| 226206_at | 5′-CACCTAATTTATTGCCGTGCGTCCT-3′ | SEQ ID NO: 659 |
| 226206_at | 5′-GCCGGGGAAGCACGGTGTGTTCTGC-3′ | SEQ ID NO: 660 |
| 226206_at | 5′-GTGACTGCTTTTGTACCTTTGCAAT-3′ | SEQ ID NO: 661 |
| 226206_at | 5′-TGCGGCCACCACCTAATTTATTGCC-3′ | SEQ ID NO: 662 |
| 226206_at | 5′-TGTGCTACTTGGCAGTTCCATTTCA-3′ | SEQ ID NO: 663 |
| 226206_at | 5′-TTCTTGGTGTCCACGTCTTGTGGGC-3′ | SEQ ID NO: 664 |
| 226206_at | 5′-TTTTGTGCTGCTTTTTATCATGATA-3′ | SEQ ID NO: 665 |
| 226420_at | 5′-AAATAGCACTGTTCCAGTCAGCCAC-3′ | SEQ ID NO: 666 |
| 226420_at | 5′-AATGAAGTGTTCCCAACCTTATGTT-3′ | SEQ ID NO: 667 |
| 226420_at | 5′-ACTCCATATTTTATGCTGGTTGTCT-3′ | SEQ ID NO: 668 |
| 226420_at | 5′-ACTGTATTCAGTTATTTTGCCCTTT-3′ | SEQ ID NO: 669 |
| 226420_at | 5′-ACTTTATGACGTCTGAGGCACACCC-3′ | SEQ ID NO: 670 |
| 226420_at | 5′-ATGGTGTTTGGCTTTTCTTAACATT-3′ | SEQ ID NO: 671 |
| 226420_at | 5′-GCCTTTCAGTGCATTACTATGGGAG-3′ | SEQ ID NO: 672 |
| 226420_at | 5′-GTCAGCCACTACTTTATGACGTCTG-3′ | SEQ ID NO: 673 |
| 226420_at | 5′-GTTGTCTGCAAGCTTGTGCGATGTT-3′ | SEQ ID NO: 674 |
| 226420_at | 5′-TGAGGTACTTTCTTCAAATGCTTTG-3′ | SEQ ID NO: 675 |
| 226420_at | 5′-TTTTGCCCTTTATTGAGGAACCAGA-3′ | SEQ ID NO: 676 |
| 229344_x_at | 5′-AATGCACCGGTTTGGATTCAGGCAC-3′ | SEQ ID NO: 677 |
| 229344_x_at | 5′-ATAACTCCAACCTGTTTGATTCCGT-3′ | SEQ ID NO: 678 |
| 229344_x_at | 5′-CTTCCCCCAATAATGCAGCTGTATA-3′ | SEQ ID NO: 679 |
| 229344_x_at | 5′-CTTCTGCGTCTGTGAGGCCAATGCA-3′ | SEQ ID NO: 680 |
| 229344_x_at | 5′-GACTAAGATTCCTGCATTTTGACTC-3′ | SEQ ID NO: 681 |
| 229344_x_at | 5′-GAGGCCAATGCAAATCCTTTTCAGG-3′ | SEQ ID NO: 682 |
| 229344_x_at | 5′-GATTTGACTGTGTGCTTTTTCAAGT-3′ | SEQ ID NO: 683 |
| 229344_x_at | 5′-GTTTGATTCCGTCTGTTTTCTAAAT-3′ | SEQ ID NO: 684 |
| 229344_x_at | 5′-TCCCCCTTCCTGATGATGAGTGAGA-3′ | SEQ ID NO: 685 |
| 229344_x_at | 5′-TGAGAACTTTCGGGGTCAGTGCCCT-3′ | SEQ ID NO: 686 |
| 229344_x_at | 5′-TTTTTTGCTTACCCTCATCAACAGA-3′ | SEQ ID NO: 687 |
| 235490_at | 5′-CAGGAGTGCACGGCGCAGATGTATA-3′ | SEQ ID NO: 688 |
| 235490_at | 5′-GATAACTTTAATCCTCACTTCTCAG-3′ | SEQ ID NO: 689 |
| 235490_at | 5′-GCACATCAGTAAATATCTGCAGTCT-3′ | SEQ ID NO: 690 |
| 235490_at | 5′-GTCGTTTGATAACTTTAATCCTCAC-3′ | SEQ ID NO: 691 |
| 235490_at | 5′-GTGCACGGCGCAGATGTATATACAT-3′ | SEQ ID NO: 692 |
| 235490_at | 5′-GTGGTTGCCCTCAGGATGGTATTCA-3′ | SEQ ID NO: 693 |
| 235490_at | 5′-TAATACAAATGGGCTCTTTGTTTTT-3′ | SEQ ID NO: 694 |
| 235490_at | 5′-TCTGCAGTCTTGTGCACATGGTGGT-3′ | SEQ ID NO: 695 |
| 235490_at | 5′-TTAATCCTCACTTCTCAGGAAACAT-3′ | SEQ ID NO: 696 |
| 235490_at | 5′-TTCTCAGGAAACATTGCACATCAGT-3′ | SEQ ID NO: 697 |
| 235490_at | 5′-TTGGTCTGTCGCCAAGGCAGGAGTG-3′ | SEQ ID NO: 698 |
| 239328_at | 5′-AAATGGTAGCAACAGACAGCCCTCT-3′ | SEQ ID NO: 699 |
| 239328_at | 5′-AGCATGGAATTGTCTACGCCTTTTG-3′ | SEQ ID NO: 700 |
| 239328_at | 5′-CTTTTGATTGGAATGCACTCCCCCT-3′ | SEQ ID NO: 701 |
| 239328_at | 5′-GAAAGACCATCAATCCTGGGTTTTA-3′ | SEQ ID NO: 702 |
| 239328_at | 5′-GGAGGTGAACGTCTTTGTGGCTATG-3′ | SEQ ID NO: 703 |
| 239328_at | 5′-GGTGAAAGTCGGCCTGTGAGTAACA-3′ | SEQ ID NO: 704 |
| 239328_at | 5′-TAGGTTCAGGGTCAGTTACCAGCCT-3′ | SEQ ID NO: 705 |
| 239328_at | 5′-TCACCATTCTTTCCCATAAGGCTTG-3′ | SEQ ID NO: 706 |
| 239328_at | 5′-TCAGTTCCGTGCTCTGTAAAACCGA-3′ | SEQ ID NO: 707 |
| 239328_at | 5′-TGCTTTACCTACCTTCCAAGGTTAT-3′ | SEQ ID NO: 708 |
| 239328_at | 5′-TTGTACATAAGCCCTACCTTTTGTC-3′ | SEQ ID NO: 709 |
| 239451_at | 5′-ACACCTATCCAGGACCTAGTTTCCA-3′ | SEQ ID NO: 710 |
| 239451_at | 5′-AGGATAGGGCAATCATTCCCAAGGA-3′ | SEQ ID NO: 711 |
| 239451_at | 5′-ATTTTGTTGGAAGCTCCATTCCCAA-3′ | SEQ ID NO: 712 |
| 239451_at | 5′-CAGGACCTAGTTTCCATGACCATGC-3′ | SEQ ID NO: 713 |
| 239451_at | 5′-CCCTTTTCTCATTGTCCATGTGATC-3′ | SEQ ID NO: 714 |
| 239451_at | 5′-GAACGATGGCTGCTAACACCTATCC-3′ | SEQ ID NO: 715 |
| 239451_at | 5′-GCTCCATTCCCAAAGCTTAACACTT-3′ | SEQ ID NO: 716 |
| 239451_at | 5′-TAAACAGGACAGTTCCATGCAGGGA-3′ | SEQ ID NO: 717 |
| 239451_at | 5′-TCCTTTGCCCACTTCTTAAATGTTA-3′ | SEQ ID NO: 718 |
| 239451_at | 5′-TTCTCCAAGTTAAGTTTCAGCCCTT-3′ | SEQ ID NO: 719 |
| 239451_at | 5′-TTTATGTAGTCTTATCCACTGCCAC-3′ | SEQ ID NO: 720 |
| 241756_at | 5′-AAACTTCTTAATTATGGAGGTACAT-3′ | SEQ ID NO: 721 |
| 241756_at | 5′-AAGAAGAAATCTTACCTTGCTCTGT-3′ | SEQ ID NO: 722 |
| 241756_at | 5′-AAGCCCATTTCTAATTGGTGATTGT-3′ | SEQ ID NO: 723 |
| 241756_at | 5′-AGAAATCTTACCTTGCTCTGTATCT-3′ | SEQ ID NO: 724 |
| 241756_at | 5′-ATGGAGGTACATCTCCAATACCTAA-3′ | SEQ ID NO: 725 |
| 241756_at | 5′-CATCCCCCTGTCAAAATGTTTGCTT-3′ | SEQ ID NO: 726 |
| 241756_at | 5′-GGCACACACTGTAGTTTCCTAAGCA-3′ | SEQ ID NO: 727 |
| 241756_at | 5′-GTACATCTCCAATACCTAAAATTAA-3′ | SEQ ID NO: 728 |
| 241756_at | 5′-GTATTGTCATTTAAGCCCATTTCTA-3′ | SEQ ID NO: 729 |
| 241756_at | 5′-GTTTCCTAAGCAGTTTGTTCTAATT-3′ | SEQ ID NO: 730 |
| 241756_at | 5′-TTCACATCCCCCTGTCAAAATGTTT-3′ | SEQ ID NO: 731 |
| 244447_at | 5′-AAAGACCTCATACCATACCTGTAAT-3′ | SEQ ID NO: 732 |
| 244447_at | 5′-AATGGTAGTAGGTGTGCCTCTCTCC-3′ | SEQ ID NO: 733 |
| 244447_at | 5′-ATTGCCACTACTGTGAGGTTTGGGT-3′ | SEQ ID NO: 734 |
| 244447_at | 5′-CAGTTGCAGGTAGCTACTCTGGAAA-3′ | SEQ ID NO: 735 |
| 244447_at | 5′-CCTCTCTCCCATGAACGGATATCGC-3′ | SEQ ID NO: 736 |
| 244447_at | 5′-GTCAGAACCCATAACAACAGGCCAG-3′ | SEQ ID NO: 737 |
| 244447_at | 5′-GTCTTAGTCCCCTTAATGGTAGTAG-3′ | SEQ ID NO: 738 |
| 244447_at | 5′-GTGTAGCTGAACTTCCTTAGTATCA-3′ | SEQ ID NO: 739 |
| 244447_at | 5′-TCTTCTTAGCCAAATACTTCTCCTT-3′ | SEQ ID NO: 740 |
| 244447_at | 5′-TGCCGCACTCTTAGTTTTTTTGCCC-3′ | SEQ ID NO: 741 |
| 244447_at | 5′-TTGATAATTTTCGTCTTAGTCCCCT-3′ | SEQ ID NO: 742 |
| 41577_at | 5′-AACTCTGTATACTGTATCAGCAGCT-3′ | SEQ ID NO: 743 |
| 41577_at | 5′-AATTCACCAGACCAGAAGCCACTGG-3′ | SEQ ID NO: 744 |
| 41577_at | 5′-ACACCCAGGAAAAGTCTGCAGACCC-3′ | SEQ ID NO: 745 |
| 41577_at | 5′-ACATGTCCCTGGAGTTGCTTCCAGC-3′ | SEQ ID NO: 746 |
| 41577_at | 5′-ACTGTATCAGCAGCTTTGTGTAAAA-3′ | SEQ ID NO: 747 |
| 41577_at | 5′-ATGGGCATTGCAAGTGCCACCGTGC-3′ | SEQ ID NO: 748 |
| 41577_at | 5′-CACCAGACCAGAAGCCACTGGTGTA-3′ | SEQ ID NO: 749 |
| 41577_at | 5′-CAGAAGCCACTGGTGTACAGAGAAC-3′ | SEQ ID NO: 750 |
| 41577_at | 5′-CCACTGGTGTACAGAGAACACTTAA-3′ | SEQ ID NO: 751 |
| 41577_at | 5′-CCCAAAGGGGGCACATGTCCCTGGA-3′ | SEQ ID NO: 752 |
| 41577_at | 5′-CGCAATAATTCACCAGACCAGAAGC-3′ | SEQ ID NO: 753 |
| 41577_at | 5′-CTTCCCATGGGCATTGCAAGTGCCA-3′ | SEQ ID NO: 754 |
| 41577_at | 5′-GAGGTAACTTCCACGTAGCCCCTTG-3′ | SEQ ID NO: 755 |
| 41577_at | 5′-GCCTGGCTCTGCACACCCAGGAAAA-3′ | SEQ ID NO: 756 |
| 41577_at | 5′-GCCTGTGACAGAATTCGCTGTTAAG-3′ | SEQ ID NO: 757 |
| 41577_at | 5′-TTTGATATCGTACTGAGGTAACTTC-3′ | SEQ ID NO: 758 |

TABLE 4
HSC gene signature

| | | | Entrez | | Representative |
|---|---|---|---|---|---|
| | | | Gene | | Public ID |
| Probe Set ID | Gene Symbol | Gene Title | ID | UniGene ID | NCBI Accession |
| 200672_x_at | SPTBN1 | spectrin, beta, non-erythrocytic 1 | 6711 | Hs.503178 | NM_003128 |
| 201889_at | FAM3C | family with sequence similarity 3, member C | 10447 | Hs.434053 | NM_014888 |
| 202551_s_at | CRIM1 | cysteine rich transmembrane BMP regulator 1 (chordin-like) | 51232 | Hs.699247 | BG546884 |
| 203139_at | DAPK1 | death-associated protein kinase 1 | 1612 | Hs.380277 | NM_004938 |
| 204069_at | MEIS1 | Meis homeobox 1 | 4211 | Hs.526754 | NM_002398 |
| 204304_s_at | PROM1 | prominin 1 | 8842 | Hs.614734 | NM_006017 |
| 204753_s_at | HLF | hepatic leukemia factor | 3131 | Hs.196952 | AI810712 |
| 204754_at | HLF | hepatic leukemia factor | 3131 | Hs.196952 | W60800 |
| 204755_x_at | HLF | hepatic leukemia factor | 3131 | Hs.196952 | M95585 |
| 204917_s_at | MLLT3 | myeloid/lymphoid or mixed-lineage leukemia (trithorax | 4300 | Hs.591085 | AV756536 |
| | | homolog, Drosophila); translocated to, 3 | | | |
| 205376_at | INPP4B | inositol polyphosphate-4-phosphatase, type II, 105 kDa | 8821 | Hs.658245 | NM_003866 |
| 205984_at | CRHBP | corticotropin releasing hormone binding protein | 1393 | Hs.115617 | NM_001882 |
| 206385_s_at | ANK3 | ankyrin 3, node of Ranvier (ankyrin 6) | 288 | Hs.499725 | NM_020987 |
| 206478_at | KIAA0125 | KIAA0125 | 9834 | Hs.649259 | NM_014792 |
| 206683_at | ZNF165 | zinc finger protein 165 | 7718 | Hs.535177 | NM_003447 |
| 208892_s_at | DUSP6 | dual specificity phosphatase 6 | 1848 | Hs.298654 | BC003143 |
| 209487_at | RBPMS | RNA binding protein with multiple splicing | 11030 | Hs.334587 | D84109 |
| 209560_s_at | DLK1 | delta-like 1 homolog (Drosophila) | 8788 | Hs.533717 | U15979 |
| 209993_at | ABCB1 | ATP-binding cassette, sub-family B (MDR/TAP), member 1 | 5243 | Hs.489033 | AF016535 |
| 211597_s_at | HOPX | HOP homeobox | 84525 | Hs.654864 | AB059408 |
| 212071_s_at | SPTBN1 | spectrin, beta, non-erythrocytic 1 | 6711 | Hs.705692 | BE968833 |
| 212488_at | COL5A1 | collagen, type V, alpha 1 | 1289 | Hs.210283 | N30339 |
| 212750_at | PPP1R16B | protein phosphatase 1, regulatory (inhibitor) subunit 16B | 26051 | Hs.45719 | AB020630 |
| 213094_at | GPR126 | G protein-coupled receptor 126 | 57211 | Hs.715560 | AL033377 |
| 213510_x_at | LOC220594 | TL132 protein | 220594 | Hs.462475 | AW194543 |
| 213844_at | HOXA5 | homeobox A5 | 3202 | Hs.655218 | NM_019102 |
| 218379_at | RBM7 | RNA binding motif protein 7 | 10179 | — | NM_016090 |
| 218723_s_at | C13orf15 | chromosome 13 open reading frame 15 | 28984 | Hs.507866 | NM_014059 |
| 218899_s_at | BAALC | brain and acute leukemia, cytoplasmic | 79870 | Hs.533446 | NM_024812 |
| 218966_at | MYO5C | myosin VC | 55930 | Hs.487036 | NM_018728 |
| 219497_s_at | BCL11A | B-cell CLL/lymphoma 11A (zinc finger protein) | 53335 | Hs.370549 | NM_022893 |
| 220416_at | ATP8B4 | ATPase, class I, type 8B, member 4 | 79895 | Hs.511311 | NM_024837 |
| 221841_s_at | KLF4 | Kruppel-like factor 4 (gut) | 9314 | Hs.376206 | BF514079 |
| 222164_at | FGFR1 | fibroblast growth factor receptor 1 | 2260 | Hs.264887 | AU145411 |
| 41577_at | PPP1R16B | protein phosphatase 1, regulatory (inhibitor) subunit 16B | 26051 | Hs.45719 | AB020630 |
| 226206_at | MAFK | v-maf musculoaponeurotic fibrosarcoma oncogene homolog K | 7975 | Hs.520612 | BG231691 |
| | | (avian) | | | |
| 226420_at | MECOM | MDS1 and EVI1 complex locus | 2122 | Hs.719216 | BG261252 |
| 229344_x_at | RIMKLB | ribosomal modification protein rimK-like family member B | 57494 | Hs.504670 | AW135012 |
| 235490_at | TMEM107 | transmembrane protein 107 | 84314 | Hs.513933 | AV743951 |
| 239328_at | — | — | — | Hs.668429 | AW512339 |
| 239451_at | — | — | — | Hs.658060 | AI684643 |
| 241756_at | — | — | — | Hs.655362 | T51136 |
| 244447_at | — | — | — | Hs.666767 | AW292830 |

TABLE 5
LSC probe set (48)

| | | |
|---|---|---|
| Probe Set ID | probe sequence | Sequence ID No. |

| | | |
|---|---|---|
| 201242_s_at | 5′-AACCTACTAGTCTTGAACAAACTGT-3′ | SEQ ID NO: 1 |
| 201242_s_at | 5′-AACTGTCATACGTATGGGACCTACA-3′ | SEQ ID NO: 2 |
| 201242_s_at | 5′-ACACTTAATCTATATGCTTTACACT-3′ | SEQ ID NO: 3 |
| 201242_s_at | 5′-AGAGCTGATCACAAGCACAAATCTT-3′ | SEQ ID NO: 4 |
| 201242_s_at | 5′-ATATGCTTTACACTAGCTTTCTGCA-3′ | SEQ ID NO: 5 |
| 201242_s_at | 5′-CTTTCCCACTAGCCATTTAATAAGT-3′ | SEQ ID NO: 6 |
| 201242_s_at | 5′-GCTTTACACTAGCTTTCTGCATTTA-3′ | SEQ ID NO: 7 |
| 201242_s_at | 5′-GCTTTCTGCATTTAATAGGTTAGAA-3′ | SEQ ID NO: 8 |
| 201242_s_at | 5′-GGACCTACACTTAATCTATATGCTT-3′ | SEQ ID NO: 9 |
| 201242_s_at | 5′-GTATGGGACCTACACTTAATCTATA-3′ | SEQ ID NO: 10 |
| 201242_s_at | 5′-TGATCACAAGCACAAATCTTTCCCA-3′ | SEQ ID NO: 11 |
| 201243_s_at | 5′-AAGCTGTGTCTGAGATCTGGATCTG-3′ | SEQ ID NO: 12 |
| 201243_s_at | 5′-CTTGTCCTCCGGTATGTTCTAAAGC-3′ | SEQ ID NO: 13 |
| 201243_s_at | 5′-GAATGCTGTCTTGACATCTCTTGCC-3′ | SEQ ID NO: 14 |
| 201243_s_at | 5′-GACTGGTGTTAAATGTTGTCTACAG-3′ | SEQ ID NO: 15 |
| 201243_s_at | 5′-GAGGCATCACATGCTGGTGCTGTGT-3′ | SEQ ID NO: 16 |
| 201243_s_at | 5′-GATCTTGTATTCAGTCAGGTTAAAA-3′ | SEQ ID NO: 17 |
| 201243_s_at | 5′-GGTGATGGGTTGTGTTATGCTTGTA-3′ | SEQ ID NO: 18 |
| 201243_s_at | 5′-GGTGCTGTGTCTTTATGAATGTTTT-3′ | SEQ ID NO: 19 |
| 201243_s_at | 5′-GTTATGCTTGTATTGAATGCTGTCT-3′ | SEQ ID NO: 20 |
| 201243_s_at | 5′-TCCGGTATGTTCTAAAGCTGTGTCT-3′ | SEQ ID NO: 21 |
| 201243_s_at | 5′-TCTGAGATCTGGATCTGCCCATCAC-3′ | SEQ ID NO: 22 |
| 201702_s_at | 5′-ACAACACCTAATGCCACCAAAGAGA-3′ | SEQ ID NO: 23 |
| 201702_s_at | 5′-AGAGGTGAAGGCTGAGACCCGGGCT-3′ | SEQ ID NO: 24 |
| 201702_s_at | 5′-AGCCTATGGAGGGCCTGGGCTTTCT-3′ | SEQ ID NO: 25 |
| 201702_s_at | 5′-AGCGACTGGATGGCTGTCATCCGCT-3′ | SEQ ID NO: 26 |
| 201702_s_at | 5′-CCAAGTTCCGTTCCACTGGACTAGA-3′ | SEQ ID NO: 27 |
| 201702_s_at | 5′-CCTTCCTGAGCGACCTTTGACAGAG-3′ | SEQ ID NO: 28 |
| 201702_s_at | 5′-GAAGAGCTCCGGAAATTGGCCTCAG-3′ | SEQ ID NO: 29 |
| 201702_s_at | 5′-GAATGCCAGCACAGTGGTGGTTTCT-3′ | SEQ ID NO: 30 |
| 201702_s_at | 5′-GCAACGTAGCTGCTCCAGGAGATGC-3′ | SEQ ID NO: 31 |
| 201702_s_at | 5′-GTCATCCGCTCTCAGAGCAGTACCC-3′ | SEQ ID NO: 32 |
| 201702_s_at | 5′-TAGAGCTGGAGACACCATCCTTGGT-3′ | SEQ ID NO: 33 |
| 204028_s_at | 5′-AAAGGCTGGGGTGGGTGACTTGACT-3′ | SEQ ID NO: 34 |
| 204028_s_at | 5′-AACCTCACTGTTCAGATGGGCTGTA-3′ | SEQ ID NO: 35 |
| 204028_s_at | 5′-AATATGCCCCGTTGACAGTGTTTAA-3′ | SEQ ID NO: 36 |
| 204028_s_at | 5′-ATAAATATCTTTCCCAATATGCCCC-3′ | SEQ ID NO: 37 |
| 204028_s_at | 5′-CACTCAAGGTTCATTGGGCTCTGCT-3′ | SEQ ID NO: 38 |
| 204028_s_at | 5′-GACTAGGACTGCTGATCTGCACAAT-3′ | SEQ ID NO: 39 |
| 204028_s_at | 5′-GCAGGGTGCACATGCTGCGAGGTCT-3′ | SEQ ID NO: 40 |
| 204028_s_at | 5′-GCGTGTCTGTAAATGTCTGCGCAGG-3′ | SEQ ID NO: 41 |
| 204028_s_at | 5′-GGAGCTGTGGACAGAGCTCCCTCAC-3′ | SEQ ID NO: 42 |
| 204028_s_at | 5′-GTATGCCTGGGTACAAACCTCACTG-3′ | SEQ ID NO: 43 |
| 204028_s_at | 5′-TCCTCCCTGCCATTACGGGAGCTGT-3′ | SEQ ID NO: 44 |
| 205321_at | 5′-AAATTGCCCTTAGCCGAAGAGTTGA-3′ | SEQ ID NO: 45 |
| 205321_at | 5′-ACGGCTTCTAGGTGTACGCACTGAA-3′ | SEQ ID NO: 46 |
| 205321_at | 5′-ATGATCTGCAATATGCTGCTCCAGG-3′ | SEQ ID NO: 47 |
| 205321_at | 5′-CAAAAATTGACCCCACTTTGTGCCG-3′ | SEQ ID NO: 48 |
| 205321_at | 5′-CCCACTTTGTGCCGGGCTGACAGAA-3′ | SEQ ID NO: 49 |
| 205321_at | 5′-GAGTTAGTGCTGTCAAGGCCGATTT-3′ | SEQ ID NO: 50 |
| 205321_at | 5′-GCAAGTACTTGGTGCAGTCGGAGCT-3′ | SEQ ID NO: 51 |
| 205321_at | 5′-GCTGCTCCAGGCGGTCTTATTGGAG-3′ | SEQ ID NO: 52 |
| 205321_at | 5′-GGTGAACATAGGATCCCTGTCAACA-3′ | SEQ ID NO: 53 |
| 205321_at | 5′-GTCGGAGCTTTACCTGAGATATTCA-3′ | SEQ ID NO: 54 |
| 205321_at | 5′-TATTTCCTGCTTAGACGGCTTCTAG-3′ | SEQ ID NO: 55 |
| 206582_s_at | 5′-AATTGGCCTTGGGGACTACTCGGCT-3′ | SEQ ID NO: 56 |
| 206582_s_at | 5′-ACAGAAATGTGGCTCCAGTTGCTCT-3′ | SEQ ID NO: 57 |
| 206582_s_at | 5′-CCCACCTGCCCATGTGATGAAGCAG-3′ | SEQ ID NO: 58 |
| 206582_s_at | 5′-CCCACGGGACTCAGAAGTGCGCCGC-3′ | SEQ ID NO: 59 |
| 206582_s_at | 5′-CTCAGCTCCCACGGGACTCAGAAGT-3′ | SEQ ID NO: 60 |
| 206582_s_at | 5′-CTTGGATCTTGAGGGTCTGGCACAT-3′ | SEQ ID NO: 61 |
| 206582_s_at | 5′-GCCGTTGCCATGGTGGACGGACTCC-3′ | SEQ ID NO: 62 |
| 206582_s_at | 5′-GGAAAGCCCAACGACCATGGAGAGA-3′ | SEQ ID NO: 63 |
| 206582_s_at | 5′-GTCAGCCGCAGACTTTGGAAAGCCC-3′ | SEQ ID NO: 64 |
| 206582_s_at | 5′-TGGAGAGATGGGCCGTTGCCATGGT-3′ | SEQ ID NO: 65 |
| 206582_s_at | 5′-TGGCACATCCTTAATCCTGTGCCCC-3′ | SEQ ID NO: 66 |
| 207090_x_at | 5′-AAATGTGGCTAGTCCAAATTCAAAT-3′ | SEQ ID NO: 67 |
| 207090_x_at | 5′-AATGGACTAGACCTGTACTAATATA-3′ | SEQ ID NO: 68 |
| 207090_x_at | 5′-CACTAGCAACCTGTTGAGCACTTGA-3′ | SEQ ID NO: 69 |
| 207090_x_at | 5′-CCGGCTCTCACTTCATATGTTTAAA-3′ | SEQ ID NO: 70 |
| 207090_x_at | 5′-CCTCAGACTTCCGAGTGGCTGGGAT-3′ | SEQ ID NO: 71 |
| 207090_x_at | 5′-CGCCACCACACCAGGTTGATTTTTG-3′ | SEQ ID NO: 72 |
| 207090_x_at | 5′-GAAATTGAGTTATTGAGCACTGAAA-3′ | SEQ ID NO: 73 |
| 207090_x_at | 5′-GCAATTACTACTGCTAAATGTGGGA-3′ | SEQ ID NO: 74 |
| 207090_x_at | 5′-GGTAGTCACTAGCAACCTGTTGAGC-3′ | SEQ ID NO: 75 |
| 207090_x_at | 5′-GTTAAGTATCTCAATTTTTCATATT-3′ | SEQ ID NO: 76 |
| 207090_x_at | 5′-TATATGTAGCTCACGTATTTCTATT-3′ | SEQ ID NO: 77 |
| 207836_s_at | 5′-ACTTCTCAGGGCTGGAAGTCCCGTC-3′ | SEQ ID NO: 78 |
| 207836_s_at | 5′-ATCTTCAGTGGTGGCTACTGTTCTC-3′ | SEQ ID NO: 79 |
| 207836_s_at | 5′-CAGGTGTGTGATGGCGGCTGCAATC-3′ | SEQ ID NO: 80 |
| 207836_s_at | 5′-CTAGCTGTTCTACAAAACTGGAGCA-3′ | SEQ ID NO: 81 |
| 207836_s_at | 5′-GAGGCTACTTCTCAGGGCTGGAAGT-3′ | SEQ ID NO: 82 |
| 207836_s_at | 5′-GCAATCTGTCTTGTGGGTATTAATG-3′ | SEQ ID NO: 83 |
| 207836_s_at | 5′-GCTGCAATCTGTCTTGTGGGTATTA-3′ | SEQ ID NO: 84 |
| 207836_s_at | 5′-GTCTTGTGGGTATTAATGCAATCTT-3′ | SEQ ID NO: 85 |
| 207836_s_at | 5′-TCTCAGGGCTGGAAGTCCCGTCAGT-3′ | SEQ ID NO: 86 |
| 207836_s_at | 5′-TCTCTAGCTGTTCTACAAAACTGGA-3′ | SEQ ID NO: 87 |
| 207836_s_at | 5′-TGCAATCTTCAGTGGTGGCTACTGT-3′ | SEQ ID NO: 88 |
| 208993_s_at | 5′-AACTCCTCATTTAGATGGGCATCAT-3′ | SEQ ID NO: 89 |
| 208993_s_at | 5′-AATTTCTCTTGTCAATGGCCAACAG-3′ | SEQ ID NO: 90 |
| 208993_s_at | 5′-CAGATGCAGCTAGCAAACCGTTTGC-3′ | SEQ ID NO: 91 |
| 208993_s_at | 5′-CATAACAACGAAACCAACTCCTCAT-3′ | SEQ ID NO: 92 |
| 208993_s_at | 5′-CCTCTGATTCCGAAAGTGCTACTGA-3′ | SEQ ID NO: 93 |
| 208993_s_at | 5′-GAGTTGTCTCTTTCACAGAGTTGTC-3′ | SEQ ID NO: 94 |
| 208993_s_at | 5′-GATACAAATGGTTCACAGTTCTTCA-3′ | SEQ ID NO: 95 |
| 208993_s_at | 5′-GCGAGAACTTTCGTTGTCTTTGTAC-3′ | SEQ ID NO: 96 |
| 208993_s_at | 5′-GCGGAGGTACGGATACTCAGTTGTG-3′ | SEQ ID NO: 97 |
| 208993_s_at | 5′-GTGTGCCCCAAAACATGCGAGAACT-3′ | SEQ ID NO: 98 |
| 208993_s_at | 5′-GTTGTGGAGAGCTGATTCCCAAATC-3′ | SEQ ID NO: 99 |
| 209272_at | 5′-ACGTTTCCTGTATTCTAATCTATTT-3′ | SEQ ID NO: 100 |
| 209272_at | 5′-ATCTTCCAACTTCCAATATTTATCC-3′ | SEQ ID NO: 101 |
| 209272_at | 5′-CCCGAGTCTCTTACACTTTATTGTG-3′ | SEQ ID NO: 102 |
| 209272_at | 5′-GAGGTGGGACGAATGCACTTGCTTC-3′ | SEQ ID NO: 103 |
| 209272_at | 5′-GATGTCCACGTTTTTGTGACTCTTC-3′ | SEQ ID NO: 104 |
| 209272_at | 5′-GGTTACCTCAGTATTACAGCCAATA-3′ | SEQ ID NO: 105 |
| 209272_at | 5′-GTGGACCCACAGATTGCATCTTTAA-3′ | SEQ ID NO: 106 |
| 209272_at | 5′-TATAGTCCAAGGGACCATTTCTCCC-3′ | SEQ ID NO: 107 |
| 209272_at | 5′-TGCACTTGCTTCCTGTGGCAATAAA-3′ | SEQ ID NO: 108 |
| 209272_at | 5′-TTATGTTTCTAGTCTTTCAAGCTTA-3′ | SEQ ID NO: 109 |
| 209272_at | 5′-TTTATCCATTCGTTGTGGACCCACA-3′ | SEQ ID NO: 110 |
| 209487_at | 5′-AACTATTTCTTGGCGACCTTTGAGA-3′ | SEQ ID NO: 111 |
| 209487_at | 5′-AATTAGATTTGTCTCTGGGAATGTG-3′ | SEQ ID NO: 112 |
| 209487_at | 5′-CTTTCACCAAAACTATTTCTTGGCG-3′ | SEQ ID NO: 113 |
| 209487_at | 5′-GGAGCTCCCATGTTGAATTTGTTTG-3′ | SEQ ID NO: 114 |
| 209487_at | 5′-GTGTTTCTCTCCTGAGGCAAAGCCC-3′ | SEQ ID NO: 115 |
| 209487_at | 5′-GTGTTTGTAACATACCAACCTACTG-3′ | SEQ ID NO: 116 |
| 209487_at | 5′-TCTTGGCGACCTTTGAGAGATTTCA-3′ | SEQ ID NO: 117 |
| 209487_at | 5′-TGTAACATACCAACCTACTGCAGAC-3′ | SEQ ID NO: 118 |
| 209487_at | 5′-TTGTCCACTTCTCCAGCAAATTAGA-3′ | SEQ ID NO: 119 |
| 209487_at | 5′-TTGTCTCTGGGAATGTGTTTGTAAC-3′ | SEQ ID NO: 120 |
| 209487_at | 5′-TTTTGTCCACTTCTCCAGCAAATTA-3′ | SEQ ID NO: 121 |
| 209488_s_at | 5′-AAGCTCACATCTAAACAGCCTGTAG-3′ | SEQ ID NO: 122 |
| 209488_s_at | 5′-AATTCCGCAAACACTACGACTAGAG-3′ | SEQ ID NO: 123 |
| 209488_s_at | 5′-ACTGTACCTCAGTTCATTGCCAGAG-3′ | SEQ ID NO: 124 |
| 209488_s_at | 5′-CAAGAACAAACTCGTAGGGACTCCA-3′ | SEQ ID NO: 125 |
| 209488_s_at | 5′-CGCTTCGATCCTGAAATTCCGCAAA-3′ | SEQ ID NO: 126 |
| 209488_s_at | 5′-GAATGCTTTGAATGGCATCCGCTTC-3′ | SEQ ID NO: 127 |
| 209488_s_at | 5′-GCCATATGAGCTCACAGTGCCTGCA-3′ | SEQ ID NO: 128 |
| 209488_s_at | 5′-GTCAGTTTTGACAGTCGCTCAGAAG-3′ | SEQ ID NO: 129 |
| 209488_s_at | 5′-TAGCCCTGAAGTGTGGGCCCCGTAC-3′ | SEQ ID NO: 130 |
| 209488_s_at | 5′-TCTGTACCCAGCGGAGTTAGCGCCT-3′ | SEQ ID NO: 131 |
| 209488_s_at | 5′-TTTACCCCAGTAGCCCTGAAGTGTG-3′ | SEQ ID NO: 132 |
| 211113_s_at | 5′-AACTGCAAGCAGCCTCTCAGCTGAT-3′ | SEQ ID NO: 133 |
| 211113_s_at | 5′-CACCAGGCACCGTGGGTCCTGGATG-3′ | SEQ ID NO: 134 |
| 211113_s_at | 5′-CATTCCCCTTTCTAGCTTTAACTAG-3′ | SEQ ID NO: 135 |
| 211113_s_at | 5′-GATGAGAGGCTTCCTCAGTCCAGTC-3′ | SEQ ID NO: 136 |
| 211113_s_at | 5′-GGAAGATTAGACACTGTGGCCGAGG-3′ | SEQ ID NO: 137 |
| 211113_s_at | 5′-GGACTTCATCGTACTCGGGATTTTC-3′ | SEQ ID NO: 138 |
| 211113_s_at | 5′-GGCCGAGGGCACGTCTAGAATCGAG-3′ | SEQ ID NO: 139 |
| 211113_s_at | 5′-GGGTCCTGGATGGGGAACTGCAAGC-3′ | SEQ ID NO: 140 |
| 211113_s_at | 5′-GTCCTCAGGTACAAAATCCGGGCAG-3′ | SEQ ID NO: 141 |
| 211113_s_at | 5′-TACTCGGGATTTTCTTCATCTCCCT-3′ | SEQ ID NO: 142 |
| 211113_s_at | 5′-TAGAACCGCGTTGGGTTTGTGGGTG-3′ | SEQ ID NO: 143 |
| 212676_at | 5′-AAGACTGGTCAGCCTGCATTAGTAT-3′ | SEQ ID NO: 144 |
| 212676_at | 5′-AGAATTGCTGCTATACTGGTGGTAT-3′ | SEQ ID NO: 145 |
| 212676_at | 5′-ATATTTCACATTTATCCACACAGTA-3′ | SEQ ID NO: 146 |
| 212676_at | 5′-ATTTCTTTGTGGTACCTGCAGTTTA-3′ | SEQ ID NO: 147 |
| 212676_at | 5′-CAAAAAGATATTAATCCCTCTACTC-3′ | SEQ ID NO: 148 |
| 212676_at | 5′-GAGCATATTGGTATCTGGATGTTCC-3′ | SEQ ID NO: 149 |
| 212676_at | 5′-GAGTTTCCTGTAGTGCTGTTTCATT-3′ | SEQ ID NO: 150 |
| 212676_at | 5′-GGTGGTATGGATTATCATGGCATTG-3′ | SEQ ID NO: 151 |
| 212676_at | 5′-GTAATGCAGATCCAATTTCTTTGTG-3′ | SEQ ID NO: 152 |
| 212676_at | 5′-GTAGGGGGGCTGTTAGAATTGCTGC-3′ | SEQ ID NO: 153 |
| 212676_at | 5′-TACTCCCAGGTTCCCTTTATATGTT-3′ | SEQ ID NO: 154 |
| 212976_at | 5′-ATCTGTGTACAATTGTTTTTGCTTC-3′ | SEQ ID NO: 155 |
| 212976_at | 5′-ATGAATGCCTTCTGCATGTTGTACA-3′ | SEQ ID NO: 156 |
| 212976_at | 5′-CTTGTATAATACACTACTGCTGAGA-3′ | SEQ ID NO: 157 |
| 212976_at | 5′-GAATGGATGTGTTCGTGCATATATA-3′ | SEQ ID NO: 158 |
| 212976_at | 5′-GAGATGGCTTTCAGTTGAGTTTAAT-3′ | SEQ ID NO: 159 |
| 212976_at | 5′-GCATGTTGTACATTATCTCTAACAG-3′ | SEQ ID NO: 160 |
| 212976_at | 5′-GCATTTTTGGTGGTAAATCCCTTTG-3′ | SEQ ID NO: 161 |
| 212976_at | 5′-GCCACAGATTCAGTAGCTTTTGGTA-3′ | SEQ ID NO: 162 |
| 212976_at | 5′-GGTAAATCCCTTTGCCACAGATTCA-3′ | SEQ ID NO: 163 |
| 212976_at | 5′-GTAGCTTTTGGTAAACTTCACTGTT-3′ | SEQ ID NO: 164 |
| 212976_at | 5′-TGGGCCAATCTGGAATAGAGACATT-3′ | SEQ ID NO: 165 |
| 213056_at | 5′-AAAGCAAATGATTTCCATATTCCTG-3′ | SEQ ID NO: 166 |
| 213056_at | 5′-AAAGCTCCAAGCTGCAGTGGATTTA-3′ | SEQ ID NO: 167 |
| 213056_at | 5′-AACAACGACAAAAAGCTCCAAGCTG-3′ | SEQ ID NO: 168 |
| 213056_at | 5′-AACTGGTCCTTAGTCATTTGTATAA-3′ | SEQ ID NO: 169 |
| 213056_at | 5′-ACAAGTTTCTTGTTCATATTGTGAA-3′ | SEQ ID NO: 170 |
| 213056_at | 5′-ACTACCTCATACTTTCCTTGGAAGA-3′ | SEQ ID NO: 171 |
| 213056_at | 5′-ATTTCCATATTCCTGATTGATCTTT-3′ | SEQ ID NO: 172 |
| 213056_at | 5′-ATTTGTATAGCCTTCTAGAATCAGA-3′ | SEQ ID NO: 173 |
| 213056_at | 5′-GAAATAACCTTTTTGCATATTCTTT-3′ | SEQ ID NO: 174 |
| 213056_at | 5′-GATTTGTTAAACTGGTCCTTAGTCA-3′ | SEQ ID NO: 175 |
| 213056_at | 5′-GGCTAAAACTACCTCATACTTTCCT-3′ | SEQ ID NO: 176 |
| 214252_s_at | 5′-AATGGGACATTAGTTCAAGTAGCAA-3′ | SEQ ID NO: 177 |
| 214252_s_at | 5′-ACCTGAAATGGATGCCCCTTTCTGG-3′ | SEQ ID NO: 178 |
| 214252_s_at | 5′-ACTTGGCAACTGTACATTTCCCCAT-3′ | SEQ ID NO: 179 |
| 214252_s_at | 5′-ATCTCCGACCTGAAATGGATGCCCC-3′ | SEQ ID NO: 180 |
| 214252_s_at | 5′-ATGCCCCTTTCTGGTGTAATCAAGG-3′ | SEQ ID NO: 181 |
| 214252_s_at | 5′-GGATTCAGAAGTACATTAACTGGCA-3′ | SEQ ID NO: 182 |
| 214252_s_at | 5′-TAACTGGCAAGAACTACACAATGGA-3′ | SEQ ID NO: 183 |
| 214252_s_at | 5′-TATGCATGATGCCATTGGATTCAGA-3′ | SEQ ID NO: 184 |
| 214252_s_at | 5′-TGCTTTTTTGAGGGAATTGATGATG-3′ | SEQ ID NO: 185 |
| 214252_s_at | 5′-TGGTATGAACTTTTCCAACTTGGCA-3′ | SEQ ID NO: 186 |
| 214252_s_at | 5′-TTCTGGTGTAATCAAGGCGCTGCCT-3′ | SEQ ID NO: 187 |
| 215411_s_at | 5′-AAACCATTGCAGGTGCCAGTGTCCC-3′ | SEQ ID NO: 188 |
| 215411_s_at | 5′-AGTGGAGTCTGTGACTGCTCTGCAT-3′ | SEQ ID NO: 189 |
| 215411_s_at | 5′-ATAAAAAAAACATCCTGCTGCGGCT-3′ | SEQ ID NO: 190 |
| 215411_s_at | 5′-CAGAACACTCATGTCTACAGCTGGC-3′ | SEQ ID NO: 191 |
| 215411_s_at | 5′-GAAACCTGTTGTGCAGAGCTCTTCC-3′ | SEQ ID NO: 192 |
| 215411_s_at | 5′-GAGGCCAGGCCATGTTTGGGGCCTT-3′ | SEQ ID NO: 193 |
| 215411_s_at | 5′-GCTTGTGTATCCTCAGACCAAACTG-3′ | SEQ ID NO: 194 |
| 215411_s_at | 5′-GGCCTTGTTCTGACAGCATTCTGGC-3′ | SEQ ID NO: 195 |
| 215411_s_at | 5′-GTTAGCCAGATGCTTGTGTATCCTC-3′ | SEQ ID NO: 196 |
| 215411_s_at | 5′-TCCACACACCCTGGCTTTGAAGTGG-3′ | SEQ ID NO: 197 |
| 215411_s_at | 5′-TGGCCCCCAGGAAACCTGTTGTGCA-3′ | SEQ ID NO: 198 |
| 216262_s_at | 5′-ATCCAGGTTAACTGATGCTGCCATT-3′ | SEQ ID NO: 199 |
| 216262_s_at | 5′-CCGTGTGCCCCAGGGGGATCAGGGA-3′ | SEQ ID NO: 200 |
| 216262_s_at | 5′-CTGGTTGGCATTTCCCCATTATGTA-3′ | SEQ ID NO: 201 |
| 216262_s_at | 5′-GAACATGGCTTCATCCAGGTTAACT-3′ | SEQ ID NO: 202 |
| 216262_s_at | 5′-GCTTTGCTCTCTCTAGGTGGGCAAG-3′ | SEQ ID NO: 203 |
| 216262_s_at | 5′-GGATGCCTGTAGTAGGGAACTCTGG-3′ | SEQ ID NO: 204 |
| 216262_s_at | 5′-GTGAGGGAGCCATGCTGCTGAATTC-3′ | SEQ ID NO: 205 |
| 216262_s_at | 5′-GTGGGAGTGTGAACGGATCGCTGAA-3′ | SEQ ID NO: 206 |
| 216262_s_at | 5′-GTGTTGGGTAGGGCAGACTCTGCTT-3′ | SEQ ID NO: 207 |
| 216262_s_at | 5′-TCGCCCATCTGTTGCTGTGGGAGTG-3′ | SEQ ID NO: 208 |
| 216262_s_at | 5′-TGGGCTGAGGTGGGATTTTCCCTCC-3′ | SEQ ID NO: 209 |
| 218183_at | 5′-ATGGCATCCACGCATGGGATCTGCA-3′ | SEQ ID NO: 210 |
| 218183_at | 5′-ATGGGATCTGCAAGCTGGAGCCCTC-3′ | SEQ ID NO: 211 |
| 218183_at | 5′-CATCTCTGCACTAACTCATCTGAAT-3′ | SEQ ID NO: 212 |
| 218183_at | 5′-CGGCAGTGGCTGTAAGGTCACCTTC-3′ | SEQ ID NO: 213 |
| 218183_at | 5′-CTGTGACTGGGCCAGGGCACACGTT-3′ | SEQ ID NO: 214 |
| 218183_at | 5′-GACAGACTGGGCTGAGGCTGACAGG-3′ | SEQ ID NO: 215 |
| 218183_at | 5′-GGCTGCAGGCAGTCTACTGGCAGGA-3′ | SEQ ID NO: 216 |
| 218183_at | 5′-GGTGGCAGTCTTGGTCAGTAGTTTA-3′ | SEQ ID NO: 217 |
| 218183_at | 5′-GGTGTAGACCAGCCCTGGGATTTCC-3′ | SEQ ID NO: 218 |
| 218183_at | 5′-TCAGTGCTGATGCCATGCCAACTGC-3′ | SEQ ID NO: 219 |
| 218183_at | 5′-TCTGCACACGCAGGTTCTGGGCGAC-3′ | SEQ ID NO: 220 |
| 218907_s_at | 5′-CCTGCACACTGGGCTATTGCTTTAT-3′ | SEQ ID NO: 221 |
| 218907_s_at | 5′-CTCCACATGCTGCAAGGACAGACTG-3′ | SEQ ID NO: 222 |
| 218907_s_at | 5′-CTGGGCTATTGCTTTATCCCTATCC-3′ | SEQ ID NO: 223 |
| 218907_s_at | 5′-GAAAGGTAGGGATGGGCCAGCCTCC-3′ | SEQ ID NO: 224 |
| 218907_s_at | 5′-GAAGGGCTGTGAGCAGGTGTAAGGG-3′ | SEQ ID NO: 225 |
| 218907_s_at | 5′-GACAGTAGGCAGGCTGAGTGGCCCA-3′ | SEQ ID NO: 226 |
| 218907_s_at | 5′-GAGCAGGTGTAAGGGCTCCCACATC-3′ | SEQ ID NO: 227 |
| 218907_s_at | 5′-GCTTTATCCCTATCCTGAGAGCAGC-3′ | SEQ ID NO: 228 |
| 218907_s_at | 5′-TCAGCTGTTGGGAGACAGTAGGCAG-3′ | SEQ ID NO: 229 |
| 218907_s_at | 5′-TGCTCCAGCCTGCAACTTAGTGGAA-3′ | SEQ ID NO: 230 |
| 218907_s_at | 5′-TTAGTGGAAGGAATTACTTCCTCCT-3′ | SEQ ID NO: 231 |
| 219871_at | 5′-AACAGATTCATCATTATTCCTAAAG-3′ | SEQ ID NO: 232 |
| 219871_at | 5′-AGTGCCTACTTTTCTTCGATATCAT-3′ | SEQ ID NO: 233 |
| 219871_at | 5′-GAACATTGTCATTTAGCCAAGCAAA-3′ | SEQ ID NO: 234 |
| 219871_at | 5′-GAGATTTCTCATATGTTTGCGTATA-3′ | SEQ ID NO: 235 |
| 219871_at | 5′-GAGCCAGCAGGTTCACCAGAAAGCT-3′ | SEQ ID NO: 236 |
| 219871_at | 5′-GAGCGTTTGCTGGAACACATTATGC-3′ | SEQ ID NO: 237 |
| 219871_at | 5′-GATATCATTAGCTGTTTTTCGAAAC-3′ | SEQ ID NO: 238 |
| 219871_at | 5′-GGAGCCAGTCGAAGATCCTGTTCAA-3′ | SEQ ID NO: 239 |
| 219871_at | 5′-GGCAGGCATTTCTTGAACATTGTCA-3′ | SEQ ID NO: 240 |
| 219871_at | 5′-TAGAAAGTATCCACCAGTGCCTACT-3′ | SEQ ID NO: 241 |
| 219871_at | 5′-TATGCTTCTGTGGCAGGCATTTCTT-3′ | SEQ ID NO: 242 |
| 220128_s_at | 5′-ACAGCCCCTGCACAAGGCTGACACA-3′ | SEQ ID NO: 243 |
| 220128_s_at | 5′-ACTAATGCTATCAAAGTCCTCCTTT-3′ | SEQ ID NO: 244 |
| 220128_s_at | 5′-AGCCCGGCTGCTCTAGCAGGAATGT-3′ | SEQ ID NO: 245 |
| 220128_s_at | 5′-AGGACTCTGCTTGTTTCAGTAGCCC-3′ | SEQ ID NO: 246 |
| 220128_s_at | 5′-CCTTGACTGGTGGGCTTTTTACGTG-3′ | SEQ ID NO: 247 |
| 220128_s_at | 5′-GCTTCTCCCACGGGTAGTGTCAGTT-3′ | SEQ ID NO: 248 |
| 220128_s_at | 5′-GGACCTCTCCCTAGTGATTATCTAG-3′ | SEQ ID NO: 249 |
| 220128_s_at | 5′-TAAGACACCTTTTATAAGCCTCCCT-3′ | SEQ ID NO: 250 |
| 220128_s_at | 5′-TACATTTGCGGTTTGGCCACAGGTC-3′ | SEQ ID NO: 251 |
| 220128_s_at | 5′-TTAAAAAGTCACTTCAGCCCCACAA-3′ | SEQ ID NO: 252 |
| 220128_s_at | 5′-TTATCTAGCCAGCTACACCTTACTC-3′ | SEQ ID NO: 253 |
| 221621_at | 5′-AAGTGTATATTGACATTTCTGGAAT-3′ | SEQ ID NO: 254 |
| 221621_at | 5′-CACTCACAAGAGTGTATACCCTGTG-3′ | SEQ ID NO: 255 |
| 221621_at | 5′-GCACAATTTGGGCCACTCACAAGAG-3′ | SEQ ID NO: 256 |
| 221621_at | 5′-GGAAATGTATTAATTGCCCAAAGTA-3′ | SEQ ID NO: 257 |
| 221621_at | 5′-GGCAGGAGAGCCGAGGTAAGACTTA-3′ | SEQ ID NO: 258 |
| 221621_at | 5′-GGTAAGACTTACTGTAGGCTGTCGT-3′ | SEQ ID NO: 259 |
| 221621_at | 5′-GTTTTTTGTCTTTGCGATGGAGTCT-3′ | SEQ ID NO: 260 |
| 221621_at | 5′-TAAACAGTTACCTACATTCTCCTCT-3′ | SEQ ID NO: 261 |
| 221621_at | 5′-TACTGTAGGCTGTCGTTTTTTTTGT-3′ | SEQ ID NO: 262 |
| 221621_at | 5′-TCCTCTGCATGCTTGTCTTTAGAGG-3′ | SEQ ID NO: 263 |
| 221621_at | 5′-TGTTTGCACAATTTGGGCCACTCAC-3′ | SEQ ID NO: 264 |
| 41113_at | 5′-AAGTCGTAGGGCAGCTATGGAAACC-3′ | SEQ ID NO: 265 |
| 41113_at | 5′-AGAAGCCTTCACCTTCCAGCTTTTG-3′ | SEQ ID NO: 266 |
| 41113_at | 5′-AGACAAGCAGTGTGATAGAGTCCTT-3′ | SEQ ID NO: 267 |
| 41113_at | 5′-AGTCACTGTATATACGTGCACATTT-3′ | SEQ ID NO: 268 |
| 41113_at | 5′-CCAGCTTTTGTCTGGCCTGTGCTGC-3′ | SEQ ID NO: 269 |
| 41113_at | 5′-CGTGGGAGCCACTGGTCTGTGCACA-3′ | SEQ ID NO: 270 |
| 41113_at | 5′-GCCTGGGATGCTCCATTGCATTTGT-3′ | SEQ ID NO: 271 |
| 41113_at | 5′-GGCAGCTATGGAAACCACTGGGTTC-3′ | SEQ ID NO: 272 |
| 41113_at | 5′-GGTGGGTTTAGTCATCTCGGAAGTC-3′ | SEQ ID NO: 273 |
| 41113_at | 5′-GTCCTTGGTGGGTTTAGTCATCTCG-3′ | SEQ ID NO: 274 |
| 41113_at | 5′-TCACCTAGTCACTGTATATACGTGC-3′ | SEQ ID NO: 275 |
| 41113_at | 5′-TCGTAGGGCAGCTATGGAAACCACT-3′ | SEQ ID NO: 276 |
| 41113_at | 5′-TCTCGGAAGTCGTAGGGCAGCTATG-3′ | SEQ ID NO: 277 |
| 41113_at | 5′-TGAGTGGCCAAGACAAGCAGTGTGA-3′ | SEQ ID NO: 278 |
| 41113_at | 5′-TGGTCTGTGCACATCCACGGTGGGT-3′ | SEQ ID NO: 279 |
| 41113_at | 5′-TTCATCCCAGCCTGGGATGCTCCAT-3′ | SEQ ID NO: 280 |
| 202646_s_at | 5′-ATAAGTAGCCGCCTGGTTACTGTGT-3′ | SEQ ID NO: 759 |
| 202646_s_at | 5′-CGCCTGGTTACTGTGTCCTGTAAAA-3′ | SEQ ID NO: 760 |
| 202646_s_at | 5′-AAAATACAGACACTTGACCCTTGGT-3′ | SEQ ID NO: 761 |
| 202646_s_at | 5′-CCTTGGTGTAGCTTCTGTTCAACTT-3′ | SEQ ID NO: 762 |
| 202646_s_at | 5′-TGGATGGGTCTGATTTCTTGGCCCT-3′ | SEQ ID NO: 763 |
| 202646_s_at | 5′-TTCTTGGCCCTCTTCTTGAATTGGC-3′ | SEQ ID NO: 764 |
| 202646_s_at | 5′-GAATTGGCCATATACAGGGTCCCTG-3′ | SEQ ID NO: 765 |
| 202646_s_at | 5′-CCAGTGGACTGAAGGCTTTGTCTAA-3′ | SEQ ID NO: 766 |
| 202646_s_at | 5′-GATGTGGGGGAGGGCGGTTTTATCT-3′ | SEQ ID NO: 767 |
| 202646_s_at | 5′-TTGAGGTTTTGATCTCTGGGTAAAG-3′ | SEQ ID NO: 768 |
| 202646_s_at | 5′-GAGGCCGTTTATCTTTGTAAACACG-3′ | SEQ ID NO: 769 |
| 202956_at | 5′-GGTAGGTGGTGATTTTGAGGCTGTA-3′ | SEQ ID NO: 770 |
| 202956_at | 5′-TGAGGCTGTAACATGCCCAGAAGCT-3′ | SEQ ID NO: 771 |
| 202956_at | 5′-GAAGCTGTTGTGGCCGACACTTCAA-3′ | SEQ ID NO: 772 |
| 202956_at | 5′-GTGGCCGACACTTCAACAATAGGGA-3′ | SEQ ID NO: 773 |
| 202956_at | 5′-ATATCCCTACTGACAGTAACTACCT-3′ | SEQ ID NO: 774 |
| 202956_at | 5′-GTAACTACCTGTCACATATTTCTCT-3′ | SEQ ID NO: 775 |
| 202956_at | 5′-CTTTTGGGTGGTGGGGCTTGATGTA-3′ | SEQ ID NO: 776 |
| 202956_at | 5′-GGCATGGTTTGCGGAGGTTAGATTT-3′ | SEQ ID NO: 777 |
| 202956_at | 5′-GTGAATTGTGCTCTGATGGTTAAAA-3′ | SEQ ID NO: 778 |
| 202956_at | 5′-AGATTGTCAAGCATTCCGTATTAAC-3′ | SEQ ID NO: 779 |
| 202956_at | 5′-ATTGATTCCCATCTGGCATATTCTA-3′ | SEQ ID NO: 780 |
| 203474_at | 5′-ACTGTGATATAGGTACTCTGATTTA-3′ | SEQ ID NO: 781 |
| 203474_at | 5′-AACTTTGGACATCCTGTGATCTGTT-3′ | SEQ ID NO: 782 |
| 203474_at | 5′-GGGGGTGGGAAATTTAGCTGACTAG-3′ | SEQ ID NO: 783 |
| 203474_at | 5′-GACAAACATGTAAACCTATTTTCCT-3′ | SEQ ID NO: 784 |
| 203474_at | 5′-AAATGTCCCACTTGAATAACGTAAT-3′ | SEQ ID NO: 785 |
| 203474_at | 5′-CTGTCTTCTGGGAGTTATCAATTTT-3′ | SEQ ID NO: 786 |
| 203474_at | 5′-GAAAGTGCACTACTGCCTCATGTAA-3′ | SEQ ID NO: 787 |
| 203474_at | 5′-TACTGCCTCATGTAAAGACTCTTGC-3′ | SEQ ID NO: 788 |
| 203474_at | 5′-AAGACTCTTGCACGCAGAGCCTTTA-3′ | SEQ ID NO: 789 |
| 203474_at | 5′-GCACGCAGAGCCTTTAAGTGACTAA-3′ | SEQ ID NO: 790 |
| 203474_at | 5′-TGAATACTTCAATTGTGCCTCTCAA-3′ | SEQ ID NO: 791 |
| 205256_at | 5′-TTTTGCTAGTGTTGAATTTTCTTCT-3′ | SEQ ID NO: 792 |
| 205256_at | 5′-CAAGCCCAAGACTGCTTAACTTCCA-3′ | SEQ ID NO: 793 |
| 205256_at | 5′-GGTATGGGAGTGGGCTCTATGGGGT-3′ | SEQ ID NO: 794 |
| 205256_at | 5′-CTCTATGGGGTGGTCTGCACCCATC-3′ | SEQ ID NO: 795 |
| 205256_at | 5′-TGGGACTCTTTTCCCTAAATCCTGC-3′ | SEQ ID NO: 796 |
| 205256_at | 5′-GGCAGGGTGCACAGCATTAGTTTCA-3′ | SEQ ID NO: 797 |
| 205256_at | 5′-CGCCCCCACCTTGAATAGCTAAAGT-3′ | SEQ ID NO: 798 |
| 205256_at | 5′-GAGTTGTTGACGTCTAACTCCTTCC-3′ | SEQ ID NO: 799 |
| 205256_at | 5′-GTCTAACTCCTTCCATTAAATTAAT-3′ | SEQ ID NO: 800 |
| 205256_at | 5′-AAGTACTGACCTCCTAATATTTAAG-3′ | SEQ ID NO: 801 |
| 205256_at | 5′-GATTCTTTTATATTCCATTGTTCAG-3′ | SEQ ID NO: 802 |
| 207837_at | 5′-TCTGCTGAATACTATACCCTTCAGC-3′ | SEQ ID NO: 803 |
| 207837_at | 5′-GAATACTATACCCTTCAGCAATGGC-3′ | SEQ ID NO: 804 |
| 207837_at | 5′-TCAGCAATGGCTACTAGAAGGACGA-3′ | SEQ ID NO: 805 |
| 207837_at | 5′-CTAGAAGGACGAACAATTGCCCTCC-3′ | SEQ ID NO: 806 |
| 207837_at | 5′-AAGGACGAACAATTGCCCTCCTTTG-3′ | SEQ ID NO: 807 |
| 207837_at | 5′-TTGGAAGTACGGCTAATAGAAGCCC-3′ | SEQ ID NO: 808 |
| 207837_at | 5′-ATAGAAGCCCTAGATCCGAATAAGA-3′ | SEQ ID NO: 809 |
| 207837_at | 5′-GCCCTAGATCCGAATAAGATCCGAA-3′ | SEQ ID NO: 810 |
| 207837_at | 5′-TAAGAATATGTAATGGACCAGGCGC-3′ | SEQ ID NO: 811 |
| 207837_at | 5′-ATGTAATGGACCAGGCGCAGTGCCT-3′ | SEQ ID NO: 812 |
| 207837_at | 5′-TGATGACAGAAGTGTGAGACCAGCC-3′ | SEQ ID NO: 813 |
| 207753_at | 5′-CAACATTGAGGCAGGGCTCACTCTC-3′ | SEQ ID NO: 814 |
| 207753_at | 5′-AGGGCTCACTCTCCTAAATTGTAGG-3′ | SEQ ID NO: 815 |
| 207753_at | 5′-GACAGATCTAACTTTCCTAGTGGAA-3′ | SEQ ID NO: 816 |
| 207753_at | 5′-GTTTCAGCATGTGTGTACACCTATG-3′ | SEQ ID NO: 817 |
| 207753_at | 5′-TACACCTATGAAACCACCACAGTCA-3′ | SEQ ID NO: 818 |
| 207753_at | 5′-ACCACAGTCAAGATATCCAACACAA-3′ | SEQ ID NO: 819 |
| 207753_at | 5′-AAGATTGTCCCTTTATAATCCTCAA-3′ | SEQ ID NO: 820 |
| 207753_at | 5′-TATAATCCTCAATTTTTCCTTATCT-3′ | SEQ ID NO: 821 |
| 207753_at | 5′-TTTCCACAATTCACAAGCAACAGCA-3′ | SEQ ID NO: 822 |
| 207753_at | 5′-GGTTAATCCATTATCTTGTTGCATG-3′ | SEQ ID NO: 823 |
| 207753_at | 5′-GAATCAATTGTTTGCTCATTTGTAT-3′ | SEQ ID NO: 824 |
| 208883_at | 5′-AACCTCTGTATGCACATGATGGGAT-3′ | SEQ ID NO: 825 |
| 208883_at | 5′-AGGACATTTGAAACCCTAATTGTGA-3′ | SEQ ID NO: 826 |
| 208883_at | 5′-AGGCACTATGCTTTTATTATATAAC-3′ | SEQ ID NO: 827 |
| 208883_at | 5′-ATGCACAATGTCTTAAGTCTTCCTA-3′ | SEQ ID NO: 828 |
| 208883_at | 5′-GATATTCTCAGCCCTGTTAACACTA-3′ | SEQ ID NO: 829 |
| 208883_at | 5′-GCCTTGAGGATAGTCTTCATGTTCA-3′ | SEQ ID NO: 830 |
| 208883_at | 5′-GTAGTGACTCATTGTATTACTTAAA-3′ | SEQ ID NO: 831 |
| 208883_at | 5′-GTCTTCATGTTCAAAGGCACTATGC-3′ | SEQ ID NO: 832 |
| 208883_at | 5′-TAAAACTTATATAACACGCTGTATT-3′ | SEQ ID NO: 833 |
| 208883_at | 5′-TACATCACCTTAACCTCTGTATGCA-3′ | SEQ ID NO: 834 |
| 208883_at | 5′-TGAACCACATGATATTCTCAGCCCT-3′ | SEQ ID NO: 835 |
| 209740_s_at | 5′-ACTCTAGAGTAATGATGGTCCCTGT-3′ | SEQ ID NO: 836 |
| 209740_s_at | 5′-ATAAACACCAACGATGGCCTCTTTT-3′ | SEQ ID NO: 837 |
| 209740_s_at | 5′-CATATGTATTTGACCCTGTGGGAGG-3′ | SEQ ID NO: 838 |
| 209740_s_at | 5′-CCCCTTCCTTTGATCATTTCATGTG-3′ | SEQ ID NO: 839 |
| 209740_s_at | 5′-GATTCTCAATTGTTATGTCCACTTA-3′ | SEQ ID NO: 840 |
| 209740_s_at | 5′-GGAGTTATGCATAGACCCACTCTAG-3′ | SEQ ID NO: 841 |
| 209740_s_at | 5′-GGTCCCTGTGGTATATACTTTCTCC-3′ | SEQ ID NO: 842 |
| 209740_s_at | 5′-GGTTCTCAGAAGCCAAAATACACAA-3′ | SEQ ID NO: 843 |
| 209740_s_at | 5′-GTCCACTTATTCACTAGGTAAATTT-3′ | SEQ ID NO: 844 |
| 209740_s_at | 5′-TAAATTCCTTGTTGATGTACCCTTA-3′ | SEQ ID NO: 845 |
| 209740_s_at | 5′-TACTTTCTCCTACTCTAGCAAACAT-3′ | SEQ ID NO: 846 |
| 211536_x_at | 5′-AGGTGAGCAGTAGGTCATCCAGTCC-3′ | SEQ ID NO: 847 |
| 211536_x_at | 5′-CAACTCGAAGTCATCCATGGACCCC-3′ | SEQ ID NO: 848 |
| 211536_x_at | 5′-CACAGCCTATTCCAAGCCTAAACGG-3′ | SEQ ID NO: 849 |
| 211536_x_at | 5′-CAGCCAAGACGTAGATCCATCCAAG-3′ | SEQ ID NO: 850 |
| 211536_x_at | 5′-CCATCCCAATGGCTTATCTTACACT-3′ | SEQ ID NO: 851 |
| 211536_x_at | 5′-CCTTTCTACTTACTACCAGCAATGC-3′ | SEQ ID NO: 852 |
| 211536_x_at | 5′-GCAAAATACATCTCGCCTGGTACAG-3′ | SEQ ID NO: 853 |
| 211536_x_at | 5′-GGACCCCTGATGATTCCACAGATAC-3′ | SEQ ID NO: 854 |
| 211536_x_at | 5′-GGGAGCAGTGTGGAGAGCTTGCCCC-3′ | SEQ ID NO: 855 |
| 211536_x_at | 5′-TCTGGATGTCCCTGAGATCGTCATA-3′ | SEQ ID NO: 856 |
| 211536_x_at | 5′-TGATTACTACCTCAGGACCAACCTC-3′ | SEQ ID NO: 857 |
| 211537_x_at | 5′-AGGTGAGCAGTAGGTCATCCAGTCC-3′ | SEQ ID NO: 858 |
| 211537_x_at | 5′-CAAAAGCCTTTCTACTTACTACCAG-3′ | SEQ ID NO: 859 |
| 211537_x_at | 5′-CAACTCGAAGTCATCCATGGACCCC-3′ | SEQ ID NO: 860 |
| 211537_x_at | 5′-CAGCCAAGACGTAGATCCATCCAAG-3′ | SEQ ID NO: 861 |
| 211537_x_at | 5′-CCATCCCAATGGCTTATCTTACACT-3′ | SEQ ID NO: 862 |
| 211537_x_at | 5′-GACAAGGCACTTCATGATTCTCTGG-3′ | SEQ ID NO: 863 |
| 211537_x_at | 5′-GACCAACCTCAGAAAAGCCAACTCG-3′ | SEQ ID NO: 864 |
| 211537_x_at | 5′-GATTCTCTGGGACCGTTACATTTTG-3′ | SEQ ID NO: 865 |
| 211537_x_at | 5′-GCAAAATACATCTCGCCTGGTACAG-3′ | SEQ ID NO: 866 |
| 211537_x_at | 5′-GGACCCCTGATGATTCCACAGATAC-3′ | SEQ ID NO: 867 |
| 211537_x_at | 5′-TGATTACTACCTCAGGACCAACCTC-3′ | SEQ ID NO: 868 |
| 212114_at | 5′-AAGTAGTCCATCCTATACAGATAGC-3′ | SEQ ID NO: 869 |
| 212114_at | 5′-AGAGGGTACATACTCCTTTCTGGGG-3′ | SEQ ID NO: 870 |
| 212114_at | 5′-CCAGGGACCACTGCCTGGCATTATC-3′ | SEQ ID NO: 871 |
| 212114_at | 5′-GAATGCTCCCTACCATATAGTTGAC-3′ | SEQ ID NO: 872 |
| 212114_at | 5′-GATTATGTGTATTGATCACCCTGCA-3′ | SEQ ID NO: 873 |
| 212114_at | 5′-GTATAAGGTGGGCTTGGTCCAACAG-3′ | SEQ ID NO: 874 |
| 212114_at | 5′-TAGCTGATTAACTGTATTCCCCTTT-3′ | SEQ ID NO: 875 |
| 212114_at | 5′-TGATCACCCTGCAATCCTATTATGT-3′ | SEQ ID NO: 876 |
| 212114_at | 5′-TGCCTGGCATTATCGCATGCTGGGA-3′ | SEQ ID NO: 877 |
| 212114_at | 5′-TGGCCCCTCTACCAATAGGGCAGTA-3′ | SEQ ID NO: 878 |
| 212114_at | 5′-TTTCTTCCATACATTAGTTCCCACC-3′ | SEQ ID NO: 879 |
| 212875_s_at | 5′-AAAGCAGCCTGCACAGGGCAAGGCC-3′ | SEQ ID NO: 880 |
| 212875_s_at | 5′-CAAACCGGCCTAGACACGAAGACCA-3′ | SEQ ID NO: 881 |
| 212875_s_at | 5′-CCTCGTTCTCTCAGTTAGCAGCTGG-3′ | SEQ ID NO: 882 |
| 212875_s_at | 5′-GAAACACAATACACTGCCTCGTTCT-3′ | SEQ ID NO: 883 |
| 212875_s_at | 5′-GATTGTATTCCTCAGTAGCACTTTA-3′ | SEQ ID NO: 884 |
| 212875_s_at | 5′-GCAGCGCACCATTCATCATTTAGGC-3′ | SEQ ID NO: 885 |
| 212875_s_at | 5′-GGCAGGACACGTATCTCTGTCTGAC-3′ | SEQ ID NO: 886 |
| 212875_s_at | 5′-GGCTTGTGGTTTGTTGTTTACTCTA-3′ | SEQ ID NO: 887 |
| 212875_s_at | 5′-GTCCATGACCGTTTGCATTCGAAAC-3′ | SEQ ID NO: 888 |
| 212875_s_at | 5′-TAATCTCACGGCTCTTGATCTGGAA-3′ | SEQ ID NO: 889 |
| 212875_s_at | 5′-TTGGCCTGACGCTGGAGTGCGGTGA-3′ | SEQ ID NO: 890 |
| 213433_at | 5′-AAGTCAGCGATTATGCCGGCGGTTA-3′ | SEQ ID NO: 891 |
| 213433_at | 5′-ATGTCGGTGCACAGCTGAAAGTCAG-3′ | SEQ ID NO: 892 |
| 213433_at | 5′-ATTCCCGTCAGAGTTTGCTTTGATT-3′ | SEQ ID NO: 893 |
| 213433_at | 5′-CACTCCATGTGGTTTCAGGGTTCAG-3′ | SEQ ID NO: 894 |
| 213433_at | 5′-GCCGGCGGTTAGAAATGTGCCAGGG-3′ | SEQ ID NO: 895 |
| 213433_at | 5′-GGGTGTCATTGATGTGGGCTGAGCT-3′ | SEQ ID NO: 896 |
| 213433_at | 5′-GGTTAAAGGAGTCCGCAGCTCCCAC-3′ | SEQ ID NO: 897 |
| 213433_at | 5′-TGAGCTGGGGAACATGTCGGTGCAC-3′ | SEQ ID NO: 898 |
| 213433_at | 5′-TGGCCTGGAGGGTGACACCATGTCA-3′ | SEQ ID NO: 899 |
| 213433_at | 5′-TTATTTTTAGCTCTGCACTCCATGT-3′ | SEQ ID NO: 900 |
| 213433_at | 5′-TTCTTTATTCCCCTCTGGACTAAAG-3′ | SEQ ID NO: 901 |
| 213557_at | 5′-CAGAGGAGGCTAAGCCCGGGCAGCT-3′ | SEQ ID NO: 902 |
| 213557_at | 5′-CCCAGTGCCCAGAAACAATGCCTAG-3′ | SEQ ID NO: 903 |
| 213557_at | 5′-CCCAGTTACACACTTCCATGGTACT-3′ | SEQ ID NO: 904 |
| 213557_at | 5′-CTCATTCTCAACTCCTTAGACTCAG-3′ | SEQ ID NO: 905 |
| 213557_at | 5′-CTTTCCATACCTGTACTCACAACTA-3′ | SEQ ID NO: 906 |
| 213557_at | 5′-GTACTATATATCATTCCTTCAGAGC-3′ | SEQ ID NO: 907 |
| 213557_at | 5′-GTCCTTTGCAAACTCATTCTCAACT-3′ | SEQ ID NO: 908 |
| 213557_at | 5′-TATTTCCTATGTATTTGTCCAGTCA-3′ | SEQ ID NO: 909 |
| 213557_at | 5′-TCCTTAGACTCAGTCAAGTCCCCCA-3′ | SEQ ID NO: 910 |
| 213557_at | 5′-TGACCATTTCTATCTGTGTTCACCA-3′ | SEQ ID NO: 911 |
| 213557_at | 5′-TGTTCACCAATGTGTTCCCAGTGCC-3′ | SEQ ID NO: 912 |
| 213861_s_at | 5′-AAATTACCTTCCTATTGCATTTCCT-3′ | SEQ ID NO: 913 |
| 213861_s_at | 5′-AGGGTAGGGCTGTGGTTTACTCCTG-3′ | SEQ ID NO: 914 |
| 213861_s_at | 5′-CTTTCCTGAGCCTCTTGCTTGAATG-3′ | SEQ ID NO: 915 |
| 213861_s_at | 5′-GACATTTGTGATTCTCATTTTCTCA-3′ | SEQ ID NO: 916 |
| 213861_s_at | 5′-GATGTACTCTTTGTTCTCTAAAACC-3′ | SEQ ID NO: 917 |
| 213861_s_at | 5′-GCTTGAATGTGATTTCTTTCTCCCT-3′ | SEQ ID NO: 918 |
| 213861_s_at | 5′-TATTGCCACCTGTCAAAATCTTCAT-3′ | SEQ ID NO: 919 |
| 213861_s_at | 5′-TGGACAAATTCTCGAACCCATTCAC-3′ | SEQ ID NO: 920 |
| 213861_s_at | 5′-TGTCTAAACCCCTGAAGCCTAACAC-3′ | SEQ ID NO: 921 |
| 213861_s_at | 5′-TTGCTACGTGTATTGGACCTCTGGC-3′ | SEQ ID NO: 922 |
| 213861_s_at | 5′-TTTCTCCCTGAGACCCATAAGGTTC-3′ | SEQ ID NO: 923 |
| 214004_s_at | 5′-ACACGTGGCTCCAGATCAAAGCGGC-3′ | SEQ ID NO: 924 |
| 214004_s_at | 5′-CAAAGCGGCCAAGGACGGAGCATCC-3′ | SEQ ID NO: 925 |
| 214004_s_at | 5′-CAAAGCTCTGGGTGACACGTGGCTC-3′ | SEQ ID NO: 926 |
| 214004_s_at | 5′-CAAGAATTACAAGGAGCCCGAGCCG-3′ | SEQ ID NO: 927 |
| 214004_s_at | 5′-CCACCTGTGACCCCGTGGTGGAGGA-3′ | SEQ ID NO: 928 |
| 214004_s_at | 5′-CCTCCTCCAACAACACGTGGATCTG-3′ | SEQ ID NO: 929 |
| 214004_s_at | 5′-CGTGGATCTGCATGGTTTGCCTGAG-3′ | SEQ ID NO: 930 |
| 214004_s_at | 5′-GACGACCACTTTGCCAAAGCTCTGG-3′ | SEQ ID NO: 931 |
| 214004_s_at | 5′-GGTTTGCCTGAGCTTTGAACAGTCA-3′ | SEQ ID NO: 932 |
| 214004_s_at | 5′-GTGGTGGAGGAGCATTTCCGCAGGA-3′ | SEQ ID NO: 933 |
| 214004_s_at | 5′-TGTGGTCTCCTGAAGGGAGCGCCTC-3′ | SEQ ID NO: 934 |
| 214197_s_at | 5′-CAAGCTGTATGTGGGCAGTCGGGTG-3′ | SEQ ID NO: 935 |
| 214197_s_at | 5′-CAGTCGGGTGGTCGCCAAATACAAA-3′ | SEQ ID NO: 936 |
| 214197_s_at | 5′-CCATTTGCCGGCCACTGAAAAAGAC-3′ | SEQ ID NO: 937 |
| 214197_s_at | 5′-GAAGGCACGTGGTGGAAGTCCCGAG-3′ | SEQ ID NO: 938 |
| 214197_s_at | 5′-GCCCCATGGTACTGCTCAAGAGTGG-3′ | SEQ ID NO: 939 |
| 214197_s_at | 5′-GCTCAAGAGTGGCCAGCTTATCAAG-3′ | SEQ ID NO: 940 |
| 214197_s_at | 5′-GGAAGTCCCGAGTTGAGGAGGTGGA-3′ | SEQ ID NO: 941 |
| 214197_s_at | 5′-GGACATAGAAGACATCTCCTGCCGT-3′ | SEQ ID NO: 942 |
| 214197_s_at | 5′-GGATGGCAGCCTAGTCAGGATCCTC-3′ | SEQ ID NO: 943 |
| 214197_s_at | 5′-TAGAGGAGTATGTCACTGCCTACCC-3′ | SEQ ID NO: 944 |
| 214197_s_at | 5′-TCTCCTGCCGTGACTTCATAGAGGA-3′ | SEQ ID NO: 945 |
| 214745_at | 5′-ACTGACATGCATTATTTTCACTGTG-3′ | SEQ ID NO: 946 |
| 214745_at | 5′-GAATAGGCCGTGAGGGTGTGAGGAA-3′ | SEQ ID NO: 947 |
| 214745_at | 5′-GAATGAGGGACTTCCATCAGACTCT-3′ | SEQ ID NO: 948 |
| 214745_at | 5′-GAGTTGCCAAACTACCTGTTGTACT-3′ | SEQ ID NO: 949 |
| 214745_at | 5′-GCAATGATGTTCTTCCTGGAATTCA-3′ | SEQ ID NO: 950 |
| 214745_at | 5′-GTTCTTATCCCACCCATAATGAGAG-3′ | SEQ ID NO: 951 |
| 214745_at | 5′-TACAGACTGCGAACAACGGCTTTCA-3′ | SEQ ID NO: 952 |
| 214745_at | 5′-TGCCCTTCCCACTTTTTGGAATAGG-3′ | SEQ ID NO: 953 |
| 214745_at | 5′-TTCAGGGAACCAAGCAACTCTATTT-3′ | SEQ ID NO: 954 |
| 214745_at | 5′-TTTAGGATGTTCTTATCCCACCCAT-3′ | SEQ ID NO: 955 |
| 214745_at | 5′-TTTTGCTAATGGCTTTGTATGTAAC-3′ | SEQ ID NO: 956 |
| 214860_at | 5′-ACACCACTGAGTGCCATGCAGAGAA-3′ | SEQ ID NO: 957 |
| 214860_at | 5′-ACATTAAGTATTTTCAGCCCACTAG-3′ | SEQ ID NO: 958 |
| 214860_at | 5′-AGAGTCCGAGTGTCTTTACACCACT-3′ | SEQ ID NO: 959 |
| 214860_at | 5′-AGCATTCAACTTTTGAGGGCTACCA-3′ | SEQ ID NO: 960 |
| 214860_at | 5′-AGGACTGAAGTATCTACTCTGGGTT-3′ | SEQ ID NO: 961 |
| 214860_at | 5′-CTGCTGCACCAGCTTAACATGTGGG-3′ | SEQ ID NO: 962 |
| 214860_at | 5′-CTTTCTGGATGAGCTGTTCTGTCTG-3′ | SEQ ID NO: 963 |
| 214860_at | 5′-GAAACCTACAAGGCACCAGGCTAGA-3′ | SEQ ID NO: 964 |
| 214860_at | 5′-GAATTCCAAACTTTGAGCCGACGAA-3′ | SEQ ID NO: 965 |
| 214860_at | 5′-GATTTCAGTGGCCACCTGAGGAATC-3′ | SEQ ID NO: 966 |
| 214860_at | 5′-GTCATTTTCCTTGTATCTGGGGAGG-3′ | SEQ ID NO: 967 |
| 215557_at | 5′-ACAGAGGCATGCTACCATACCTGGT-3′ | SEQ ID NO: 968 |
| 215557_at | 5′-ACCTCCTAATACCAACACCTTGAAG-3′ | SEQ ID NO: 969 |
| 215557_at | 5′-AGAGCGGTAGGTTACTCTGGGCACA-3′ | SEQ ID NO: 970 |
| 215557_at | 5′-CAGAGGCTCTGGCTCGAAGGAAGCG-3′ | SEQ ID NO: 971 |
| 215557_at | 5′-CCAAGGCCTTCCTTGGTGTTGCCTC-3′ | SEQ ID NO: 972 |
| 215557_at | 5′-GAAGGAAGCGGAGGGCGTGGCTGCT-3′ | SEQ ID NO: 973 |
| 215557_at | 5′-GCCTTTTCTTAGTGCCTTAGAGGGC-3′ | SEQ ID NO: 974 |
| 215557_at | 5′-GGCTGCTGAGACAGCCAACACCTCT-3′ | SEQ ID NO: 975 |
| 215557_at | 5′-GTGTGCTCTTCCCAGTAGAGCGGTA-3′ | SEQ ID NO: 976 |
| 215557_at | 5′-TTGCCTCCATTCCCTGGAAAGGTCT-3′ | SEQ ID NO: 977 |
| 215557_at | 5′-TTTTACATTTCAGTGTGCTCTTCCC-3′ | SEQ ID NO: 978 |
| 219236_at | 5′-AACCAGGCCGAGAGGCCACACACTT-3′ | SEQ ID NO: 979 |
| 219236_at | 5′-ATGCATGCGTGTCCAGGCTGAAGAT-3′ | SEQ ID NO: 980 |
| 219236_at | 5′-CCATCCCCACAAACCAGGTAATGCC-3′ | SEQ ID NO: 981 |
| 219236_at | 5′-CTGAATGCTTCTTGCTAACCAGGCC-3′ | SEQ ID NO: 982 |
| 219236_at | 5′-CTTCTGGAAGTCTCTGCTCAGCACA-3′ | SEQ ID NO: 983 |
| 219236_at | 5′-GAAGATGCCCCTATATTCTGTCAAA-3′ | SEQ ID NO: 984 |
| 219236_at | 5′-GACCGTGAGGGGGCTCTTGATGGGA-3′ | SEQ ID NO: 985 |
| 219236_at | 5′-GCTCAAGGTGTCCAGGCTTTTGGGG-3′ | SEQ ID NO: 986 |
| 219236_at | 5′-GTCCTGGTCATAACTGTGTGCTCAA-3′ | SEQ ID NO: 987 |
| 219236_at | 5′-GTTTGCCAGCAGCTATTTGCCTATA-3′ | SEQ ID NO: 988 |
| 219236_at | 5′-TGGGCCTATCTGGGTGCATTATGGA-3′ | SEQ ID NO: 989 |
| 219658_at | 5′-AACAGAATACTAAGGGCCCCTACTG-3′ | SEQ ID NO: 990 |
| 219658_at | 5′-AGGTGTTTGCTGAATCCAGGTCTGA-3′ | SEQ ID NO: 991 |
| 219658_at | 5′-CACTGTACCACATTATCTCTTTTCA-3′ | SEQ ID NO: 992 |
| 219658_at | 5′-CCAGGTCTGAGATCACAATCCCACC-3′ | SEQ ID NO: 993 |
| 219658_at | 5′-CTCTGCCCTCATAGAATCCTAATTG-3′ | SEQ ID NO: 994 |
| 219658_at | 5′-GAAACATTGAACAGCCCCATTTAGA-3′ | SEQ ID NO: 995 |
| 219658_at | 5′-GAGGCCCAATCTCAACTGTAGACTG-3′ | SEQ ID NO: 996 |
| 219658_at | 5′-GATTCCTAGTCTAGTATCCTTCCCA-3′ | SEQ ID NO: 997 |
| 219658_at | 5′-GGATCCGCATATGAGAGTCGCACAT-3′ | SEQ ID NO: 998 |
| 219658_at | 5′-TACCGACCTCTTAGGCTTGGTGTGA-3′ | SEQ ID NO: 999 |
| 219658_at | 5′-TCTTGTCCTTGTGCTCTGTGAAACA-3′ | SEQ ID NO: 1000 |
| 221483_s_at | 5′-ACTTCGCCTGTACTGAAAGGGCCAA-3′ | SEQ ID NO: 1001 |
| 221483_s_at | 5′-CAACCTTCTAATTAGGTAGGCCTCT-3′ | SEQ ID NO: 1002 |
| 221483_s_at | 5′-CCCTTGGATCTGTTACTGCATCACT-3′ | SEQ ID NO: 1003 |
| 221483_s_at | 5′-GATCACTGCTGGTCTTGATAGCCAT-3′ | SEQ ID NO: 1004 |
| 221483_s_at | 5′-GATGCAATAGAACACTTCGCCTGTA-3′ | SEQ ID NO: 1005 |
| 221483_s_at | 5′-GCTGAATTTGTCAAATACCCCTTCC-3′ | SEQ ID NO: 1006 |
| 221483_s_at | 5′-TAATTTGAGCCACATTCCCAACTCT-3′ | SEQ ID NO: 1007 |
| 221483_s_at | 5′-TAGGCCTCTAGGTATTCTGCAGATC-3′ | SEQ ID NO: 1008 |
| 221483_s_at | 5′-TATCTCACTCTGTCATTGTTAATCT-3′ | SEQ ID NO: 1009 |
| 221483_s_at | 5′-TGTTACTGCATCACTAGCACTTGTG-3′ | SEQ ID NO: 1010 |
| 221483_s_at | 5′-TTCCCCACCACACCTTATAAAATTG-3′ | SEQ ID NO: 1011 |

TABLE 6
LSC gene signature (48)

| | | | Entrez | | Representative |
|---|---|---|---|---|---|
| Probe Set ID | Gene Symbol | Gene Title | Gene ID | UniGene ID | Public ID |
| 201242_s_at | ATP1B1 | “ATPase, Na+/K+ transporting, beta 1 polypeptide” | 481 | Hs.291196 | BC000006 |
| 201243_s_at | ATP1B1 | “ATPase, Na+/K+ transporting, beta 1 polypeptide” | 481 | Hs.291196 | NM_001677 |
| 201702_s_at | PPP1R10 | “protein phosphatase 1, regulatory (inhibitor) subunit 10” | 5514 | Hs.106019 | AI492873 |
| 202646_s_at | CSDE1 | “cold shock domain containing E1, RNA-binding” | 7812 | Hs.69855 | AA167775 |
| 202956_at | ARFGEF1 | ADP-ribosylation factor guanine nucleotide-exchange factor | 10565 | Hs.656902 | NM_006421 |
| | | 1(brefeldin A-inhibited) | | | |
| 203474_at | IQGAP2 | IQ motif containing GTPase activating protein 2 | 10788 | Hs.291030 | NM_006633 |
| 204028_s_at | RABGAP1 | RAB GTPase activating protein 1 | 23637 | Hs.271341 | NM_012197 |
| 205256_at | ZBTB39 | zinc finger and BTB domain containing 39 | 9880 | Hs.591025 | NM_014830 |
| 205321_at | EIF2S3 | “eukaryotic translation initiation factor 2, subunit 3 gamma, | 1968 | Hs.539684 | NM_001415 |
| | | 52 kDa” | | | |
| 206582_s_at | GPR56 | G protein-coupled receptor 56 | 9289 | Hs.513633 | NM_005682 |
| 207090_x_at | ZFP30 | zinc finger protein 30 homolog (mouse) | 22835 | Hs.716719 | NM_014898 |
| 207753_at | ZNF304 | zinc finger protein 304 | 57343 | Hs.287374 | NM_020657 |
| 207836_s_at | RBPMS | RNA binding protein with multiple splicing | 11030 | Hs.334587 | NM_006867 |
| 207837_at | RBPMS | RNA binding protein with multiple splicing | 11030 | Hs.334587 | NM_006867 |
| 208883_at | UBR5 | ubiquitin protein ligase E3 component n-recognin 5 | 51366 | Hs.591856 | BF515424 |
| 208993_s_at | PPIG | peptidylprolyl isomerase G (cyclophilin G) | 9360 | Hs.470544 | AW340788 |
| 209272_at | NAB1 | NGFI-A binding protein 1 (EGR1 binding protein 1) | 4664 | Hs.570078 | AF045451 |
| 209487_at | RBPMS | RNA binding protein with multiple splicing | 11030 | Hs.334587 | D84109 |
| 209488_s_at | RBPMS | RNA binding protein with multiple splicing | 11030 | Hs.334587 | D84109 |
| 209740_s_at | PNPLA4 | patatin-like phospholipase domain containing 4 | 8228 | Hs.264 | U03886 |
| 211113_s_at | ABCG1 | “ATP-binding cassette, sub-family G (WHITE), member 1” | 9619 | Hs.124649 | U34919 |
| 211536_x_at | MAP3K7 | mitogen-activated protein kinase kinase kinase 7 | 6885 | Hs.719192 | AB009358 |
| 211537_x_at | MAP3K7 | mitogen-activated protein kinase kinase kinase 7 | 6885 | Hs.719192 | AF218074 |
| 212114_at | LOC552889 | hypothetical protein LOC552889 | 552889 | Hs.213541 | BE967207 |
| 212676_at | NF1 | neurofibromin 1 | 4763 | Hs.113577 | AW293356 |
| 212875_s_at | C2CD2 | C2 calcium-dependent domain containing 2 | 25966 | Hs.473894 | AP001745 |
| 212976_at | LRRC8B | “leucine rich repeat containing 8 family, member B” | 23507 | Hs.482017 | R41498 |
| 213056_at | FRMD4B | FERM domain containing 4B | 23150 | Hs.709671 | AU145019 |
| 213433_at | ARL3 | ADP-ribosylation factor-like 3 | 403 | Hs.182215 | AF038193 |
| 213557_at | CRKRS | “Cdc2-related kinase, arginine/serine-rich” | 51755 | Hs.416108 | AW305119 |
| 213861_s_at | FAM119B | “family with sequence similarity 119, member B” | 25895 | Hs.632720 | N67741 |
| 214004_s_at | VGLL4 | vestigial like 4 (Drosophila) | 9686 | Hs.38032 | AI806207 |
| 214197_s_at | SETDB1 | “SET domain, bifurcated 1” | 9869 | Hs.643565 | AI762193 |
| 214252_s_at | CLN5 | “ceroid-lipofuscinosis, neuronal 5” | 1203 | Hs.30213 | AV700514 |
| 214745_at | PLCH1 | “phospholipase C, eta 1” | 23007 | Hs.567423 | AW665865 |
| 214860_at | SLC9A7 | “solute carrier family 9 (sodium/hydrogen exchanger), | 84679 | Hs.496057 | AL022165 |
| | | member 7” | | | |
| 215411_s_at | TRAF3IP2 | TRAF3 interacting protein 2 | 10758 | Hs.654708 | AL008730 |
| 215557_at | — | — | — | Hs.658129 | AU144900 |
| 216262_s_at | TGIF2 | TGFB-induced factor homeobox 2 | 60436 | Hs.632264 | AL050318 |
| 218183_at | C16orf5 | chromosome 16 open reading frame 5 | 29965 | Hs.654653 | NM_013399 |
| 218907_s_at | LRRC61 | leucine rich repeat containing 61 | 65999 | Hs.647119 | NM_023942 |
| 219236_at | PAQR6 | progestin and adipoQ receptor family member VI | 79957 | Hs.235873 | NM_024897 |
| 219658_at | PTCD2 | pentatricopeptide repeat domain 2 | 79810 | Hs.126906 | NM_024754 |
| 219871_at | FLJ13197 | hypothetical FLJ13197 | 79667 | Hs.29725 | NM_024614 |
| 220128_s_at | NIPAL2 | NIPA-like domain containing 2 | 79815 | Hs.309489 | NM_024759 |
| 221483_s_at | ARPP19 | “cAMP-regulated phosphoprotein, 19 kDa” | 10776 | Hs.512908 | AF084555 |
| 221621_at | C17orf86 | chromosome 17 open reading frame 86 | 654434 | — | AF130050 |
| 41113_at | ZNF500 | zinc finger protein 500 | 26048 | Hs.513316 | AI871396 |

TABLE 7
Summary of Patient Data

| AML | Relapse or | | | | Karyotype and Molecular |
|---|---|---|---|---|---|
| # | Diagnosis | FAB | Age | Sex | Marker |
| 1 | Relapse | M2 | 48 | f | 46, t(2; 21)(p21; q22)[4]/46, 9 |
| | (AML #9) | | | | (1; 21)(q22; q22) |
| 2 | Diag | M5a | 58 | f | normal, FLT3ITD |
| 3 | Diag | unclass | 52 | f | +8 |
| 4 | Diag | unclass | 62 | m | normal |
| 5 | Diag | M5a | 39 | f | +8 |
| 6 | Diag | unclass | 80 | f | normal |
| 7 | Diag | M5 | 48 | m | no data |
| 8 | Diag | M1 | 72 | f | normal |
| 9 | Diag | M2 | 47 | f | 46, t(2:21)[4]/t(6:21)[2]/t(15: |
| | | | | | 21)[2] |
| 10 | Diag | M2 | 62 | f | trisomy 13 |
| 11 | Diag | M1 | 45 | f | normal |
| 12 | Diag | M4eo | 39 | m | 46, inv(16)(p13; q22) |
| 13 | Diag | M5a | 40 | m | normal, FLT3ITD |
| 14 | Diag | M5a | 75 | m | normal |
| 15 | Diag | M4 | 23 | m | normal |
| 16 | Diag | M5b | 80 | m | no data |

TABLE 8
Frequency of LSC in each fraction of 16 AML

| | | | | CD34−/CD38− |
|---|---|---|---|---|
| | CD34+/CD38− | CD34+/CD38+ | CD34−/CD38+ | Frequency |
| | Frequency | Frequency | Frequency | 1 LSC per X |
| | 1 LSC per X cells | 1 LSC per X cells | 1 LSC per X cells | cells |
| AML | (95% CI) | (95% CI) | (95% CI) | (95% CI) |
| 1 | 1.6 × 10³ | 1.3 × 10⁵ | 0 | 0 |
| | (2.7 × 10²-9.9 × 10³) | (4.6 × 10⁴-3.7 × 10⁵) | | |
| 2 | 5.8 × 10³ | 4.2 × 10³ | 0 | 0 |
| | (1.8 × 10³-1.8 × 10⁴) | (1.4 × 10³-1.3 × 10⁴) | | |
| 3 | 6.2 × 10³* | 7.6 × 10³* | 9.6 × 10³ | 7.7 × 10³* |
| | (1-6.2 × 10³) | (1-7.6 × 10³) | (2.5 × 10³-3.7 × 10⁴) | (1-7.7 × 10³) |
| 4 | 7.1 × 10³ | 9.2 × 10⁴ | 0 | 4.4 × 10⁵* |
| | (1.1 × 10³-4.6 × 10⁴) | (2.7 × 10⁴-3.1 × 10⁵) | | (1-4.4 × 10⁵) |
| 5 | 1.1 × 10⁴ | 4.5 × 10⁴ | 0 | 0 |
| | (3.7 × 10³-3.4 × 10⁴) | (1.8 × 10⁴-1.2 × 10⁵) | | |
| 6 | 1.7 × 10⁵ | 1.5 × 10⁵ | 0 | 0 |
| | (6.9 × 10⁴-4.2 × 10⁵) | (5.8 × 10⁴-4.1 × 10⁵) | | |
| 7 | 1.7 × 10⁵* | nt | nt | 9.1 × 10⁵* |
| | (1-1.7 × 10⁵) | | | (1-9.1 × 10⁵) |
| 8 | 2.1 × 10⁵ | 0 | 0 | 0 |
| | (9.3 × 10⁴-4.9 × 10⁵) | | | |
| 9 | 2.6 × 10⁵* | nt | nt | nt |
| | (1-2.6 × 10⁵) | | | |
| 10 | 2.5 × 10⁵ | nt | nt | nt |
| | (6.0 × 10⁴-1.0 × 10⁶) | | | |
| 11 | 4.5 × 10⁵ | 4.9 × 10⁴ | 0 | 0 |
| | (6.4 × 10⁴-3.1 × 10⁶) | (1.9 × 10⁴-1.3 × 10⁵) | | |
| 12 | 4.9 × 10⁵ | 0 | 0 | 0 |
| | (6.9 × 10⁴-3.5 × 10⁶) | | | |
| 13 | 1.1 × 10⁶ | 2.4 × 10⁵ | 0 | 0 |
| | (2.7 × 10⁵-4.3 × 10⁶) | (9.0 × 10⁴-6.3 × 10⁵) | | |
| 14 | ** | 0 | 0 | 0 |
| 15 | *** | 0 | 0 | 0 |
| 16 | *** | 0 | 0 | 0 |
| Total | 13/14 (93%) | 8/13 (62%) | 1/13 (8%) | 3/14 (21%) |

TABLE 9
Secondary engraftment of samples with LSC in multiple fractions
Secondary Transplantation/Primary Mice

| AML | 34+ 38− | 34+ 38+ | 34− 38+ | 34− 38− |
|---|---|---|---|---|
| 1 | 3/3 | 2/2 | | |
| 2 | 3/5 | 3/6 | | |
| 4 | 3/3 | 1/2 | | 2/2 |
| 5 | 0/2 | 0/2 | | |
| 11 | 0/1 | 1/2 | | |

Note:
“Number of primary mice with secondary engraftment”/“total number of primary mice tested”

TABLE 10
Percentage of each CD34 and CD38 sorted populations
in 16 primary human AML samples

Percentage of Each Sorted Fraction

| AML | +/− | +/+ | −/+ | −/− |
|---|---|---|---|---|
| 1 | 5.9 | 80.4 | 13.1 | 0.6 |
| 2 | 15.3 | 50.3 | 31.0 | 3.4 |
| 3 | 8.6 | 65.4 | 24.1 | 2.0 |
| 4 | 10.9 | 17.2 | 62.2 | 9.7 |
| 5 | 3.7 | 18.0 | 72.4 | 5.9 |
| 6 | 90.8 | 3.0 | 2.9 | 3.3 |
| 7 | 49.8 | 15.3 | 29.0 | 5.9 |
| 8 | 1.0 | 31.1 | 66.1 | 1.9 |
| 9 | 4.2 | 62.1 | 33.1 | 0.6 |
| 10 | 4.8 | 60.0 | 19.5 | 15.7 |
| 11 | 11.8 | 39.8 | 39.8 | 8.5 |
| 12 | 1.2 | 48.6 | 48.8 | 1.5 |
| 13 | 12.3 | 5.3 | 67.0 | 15.3 |
| 14 | 0.4 | 71.3 | 22.6 | 5.8 |
| 15 | 0.1 | 46.2 | 43.3 | 10.5 |
| 16 | 0.7 | 7.7 | 86.6 | 4.9 |

TABLE 11
Percentage of total LSC in each sorted fraction of primary human AML

Percentage of Total LSC in Each Fraction*

| | +/− | +/+ | −/+ | −/− |
|---|---|---|---|---|
| AML | (%) | (%) | (%) | (%) |
| 1 | 85 | 15 | 0 | 0 |
| 2 | 18 | 82 | 0 | 0 |
| 3 | 13** | 79** | 6** | 2** |
| 4 | 75** | 18** | 0 | 7** |
| 5 | 46 | 54 | 0 | 0 |
| 6 | 96 | 4 | 0 | 0 |
| 8 | 100 | 0 | 0 | 0 |
| 11 | 3 | 97 | 0 | 0 |
| 12 | 100 | 0 | 0 | 0 |
| 13 | 33 | 67 | 0 | 0 |

*estimated by multiplying LSC frequency by the percentage of total patient cells each fraction represents
**Estimate from lower 95% interval

TABLE 12
Complete LSC-R Probe List, including FDR<=0.05

| | Gene | | Entrez | Representative |
|---|---|---|---|---|
| Probe Set ID | Symbol | Gene Title | Gene ID | Public ID |
| 201018_at | EIF1AX | eukaryotic translation | 1964 | AL079283 |
| | | initiation factor 1A, X- | | |
| | | linked | | |
| 201080_at | PIP4K2B | phosphatidylinositol-5- | 8396 | BF338509 |
| | | phosphate 4-kinase, type | | |
| | | II, beta | | |
| 201242_s_at | ATP1B1 | ATPase, Na+/K+ | 481 | BC000006 |
| | | transporting, beta 1 | | |
| | | polypeptide | | |
| 201243_s_at | ATP1B1 | ATPase, Na+/K+ | 481 | NM_001677 |
| | | transporting, beta 1 | | |
| | | polypeptide | | |
| 201702_s_at | PPP1R10 | protein phosphatase 1, | 5514 | AI492873 |
| | | regulatory (inhibitor) | | |
| | | subunit 10 | | |
| 202599_s_at | NRIP1 | nuclear receptor | 8204 | NM_003489 |
| | | interacting protein 1 | | |
| 202646_s_at | CSDE1 | cold shock domain | 7812 | AA167775 |
| | | containing E1, RNA- | | |
| | | binding | | |
| 202956_at | ARFGEF1 | ADP-ribosylation factor | 10565 | NM_006421 |
| | | guanine nucleotide- | | |
| | | exchange factor | | |
| | | 1(brefeldin A-inhibited) | | |
| 203106_s_at | VPS41 | vacuolar protein sorting | 27072 | NM_014396 |
| | | 41 homolog (S. cerevisiae) | | |
| 203474_at | IQGAP2 | IQ motif containing | 10788 | NM_006633 |
| | | GTPase activating protein 2 | | |
| 204028_s_at | RABGAP1 | RAB GTPase activating | 23637 | NM_012197 |
| | | protein 1 | | |
| 204837_at | MTMR9 | myotubularin related | 66036 | AL080178 |
| | | protein 9 | | |
| 205094_at | PEX12 | peroxisomal biogenesis | 5193 | NM_000286 |
| | | factor 12 | | |
| 205256_at | ZBTB39 | zinc finger and BTB | 9880 | NM_014830 |
| | | domain containing 39 | | |
| 205321_at | EIF2S3 | eukaryotic translation | 1968 | NM_001415 |
| | | initiation factor 2, subunit | | |
| | | 3 gamma, 52 kDa | | |
| 205608_s_at | ANGPT1 | angiopoietin 1 | 284 | U83508 |
| 205702_at | PHTF1 | putative homeodomain | 10745 | NM_006608 |
| | | transcription factor 1 | | |
| 206582_s_at | GPR56 | G protein-coupled | 9289 | NM_005682 |
| | | receptor 56 | | |
| 206874_s_at | SLK | STE20-like kinase (yeast) | 9748 | AL138761 |
| 206945_at | LCT | lactase | 3938 | NM_002299 |
| 207090_x_at | ZFP30 | zinc finger protein 30 | 22835 | NM_014898 |
| | | homolog (mouse) | | |
| 207737_at | — | — | — | NM_021981 |
| 207753_at | ZNF304 | zinc finger protein 304 | 57343 | NM_020657 |
| 207836_s_at | RBPMS | RNA binding protein with | 11030 | NM_006867 |
| | | multiple splicing | | |
| 207837_at | RBPMS | RNA binding protein with | 11030 | NM_006867 |
| | | multiple splicing | | |
| 207968_s_at | MEF2C | myocyte enhancer factor | 4208 | NM_002397 |
| | | 2C | | |
| 208634_s_at | MACF1 | microtubule-actin | 23499 | AB029290 |
| | | crosslinking factor 1 | | |
| 208883_at | UBR5 | ubiquitin protein ligase E3 | 51366 | BF515424 |
| | | component n-recognin 5 | | |
| 208993_s_at | PPIG | peptidylprolyl isomerase | 9360 | AW340788 |
| | | G (cyclophilin G) | | |
| 209200_at | MEF2C | myocyte enhancer factor | 4208 | AL536517 |
| | | 2C | | |
| 209272_at | NAB1 | NGFI-A binding protein 1 | 4664 | AF045451 |
| | | (EGR1 binding protein 1) | | |
| 209425_at | AMACR /// | alpha-methylacyl-CoA | 114899 | AA888589 |
| | C1QTNF3 | racemase /// C1q and | /// | |
| | | tumor necrosis factor | 23600 | |
| | | related protein 3 | | |
| 209487_at | RBPMS | RNA binding protein with | 11030 | D84109 |
| | | multiple splicing | | |
| 209488_s_at | RBPMS | RNA binding protein with | 11030 | D84109 |
| | | multiple splicing | | |
| 209740_s_at | PNPLA4 | patatin-like | 8228 | U03886 |
| | | phospholipase domain | | |
| | | containing 4 | | |
| 210132_at | EFNA3 | ephrin-A3 | 1944 | AW189015 |
| 211113_s_at | ABCG1 | ATP-binding cassette, | 9619 | U34919 |
| | | sub-family G (WHITE), | | |
| | | member 1 | | |
| 211255_x_at | DEDD | death effector domain | 9191 | AF064605 |
| | | containing | | |
| 211536_x_at | MAP3K7 | mitogen-activated | 6885 | AB009358 |
| | | protein kinase kinase | | |
| | | kinase 7 | | |
| 211537_x_at | MAP3K7 | mitogen-activated | 6885 | AF218074 |
| | | protein kinase kinase | | |
| | | kinase 7 | | |
| 211877_s_at | PCDHGA11 | protocadherin gamma | 56105 | AF152505 |
| | | subfamily A, 11 | | |
| 212114_at | ATXN7L3B | ataxin 7-like 3B | 552889 | BE967207 |
| 212397_at | RDX | radixin | 5962 | AL137751 |
| 212676_at | NF1 | neurofibromin 1 | 4763 | AW293356 |
| 212851_at | DCUN1D4 | DCN1, defective in cullin | 23142 | AA194584 |
| | | neddylation 1, domain | | |
| | | containing 4 (S. cerevisiae) | | |
| 212875_s_at | C2CD2 | C2 calcium-dependent | 25966 | AP001745 |
| | | domain containing 2 | | |
| 212976_at | LRRC8B | leucine rich repeat | 23507 | R41498 |
| | | containing 8 family, | | |
| | | member B | | |
| 213056_at | FRMD4B | FERM domain containing | 23150 | AU145019 |
| | | 4B | | |
| 213313_at | RABGAP1 | RAB GTPase activating | 23637 | AI922519 |
| | | protein 1 | | |
| 213433_at | ARL3 | ADP-ribosylation factor- | 403 | AF038193 |
| | | like 3 | | |
| 213557_at | CDK12 | cyclin-dependent kinase | 51755 | AW305119 |
| | | 12 | | |
| 213639_s_at | ZNF500 | zinc finger protein 500 | 26048 | AI871396 |
| 213861_s_at | FAM119B | family with sequence | 25895 | N67741 |
| | | similarity 119, member B | | |
| 214004_s_at | VGLL4 | vestigial like 4 | 9686 | AI806207 |
| | | (Drosophila) | | |
| 214197_s_at | SETDB1 | SET domain, bifurcated 1 | 9869 | AI762193 |
| 214252_s_at | CLN5 | ceroid-lipofuscinosis, | 1203 | AV700514 |
| | | neuronal 5 | | |
| 214738_s_at | NEK9 | NIMA (never in mitosis | 91754 | BE792298 |
| | | gene a)-related kinase 9 | | |
| 214745_at | PLCH1 | phospholipase C, eta 1 | 23007 | AW665865 |
| 214820_at | BRWD1 | bromodomain and WD | 54014 | AJ002572 |
| | | repeat domain containing 1 | | |
| 214860_at | SLC9A7 | solute carrier family 9 | 84679 | AL022165 |
| | | (sodium/hydrogen | | |
| | | exchanger), member 7 | | |
| 215411_s_at | TRAF3IP2 | TRAF3 interacting protein 2 | 10758 | AL008730 |
| 215557_at | — | — | — | AU144900 |
| 216262_s_at | TGIF2 | TGFB-induced factor | 60436 | AL050318 |
| | | homeobox 2 | | |
| 218183_at | C16orf5 | chromosome 16 open | 29965 | NM_013399 |
| | | reading frame 5 | | |
| 218907_s_at | LRRC61 | leucine rich repeat | 65999 | NM_023942 |
| | | containing 61 | | |
| 219232_s_at | EGLN3 | egl nine homolog 3 (C. elegans) | 112399 | NM_022073 |
| 219236_at | PAQR6 | progestin and adipoQ | 79957 | NM_024897 |
| | | receptor family member | | |
| | | VI | | |
| 219383_at | PRR5L | proline rich 5 like | 79899 | NM_024841 |
| 219658_at | PTCD2 | pentatricopeptide repeat | 79810 | NM_024754 |
| | | domain 2 | | |
| 219718_at | FGGY | FGGY carbohydrate | 55277 | NM_018291 |
| | | kinase domain containing | | |
| 219871_at | FLJ13197 | hypothetical FLJ13197 | 79667 | NM_024614 |
| 220128_s_at | NIPAL2 | NIPA-like domain | 79815 | NM_024759 |
| | | containing 2 | | |
| 220360_at | THAP9 | THAP domain containing 9 | 79725 | NM_024672 |
| 221020_s_at | SLC25A32 | solute carrier family 25, | 81034 | NM_030780 |
| | | member 32 | | |
| 221294_at | GPR21 | G protein-coupled | 2844 | NM_005294 |
| | | receptor 21 | | |
| 221483_s_at | ARPP19 | cAMP-regulated | 10776 | AF084555 |
| | | phosphoprotein, 19 kDa | | |
| 221621_at | C17orf86 | chromosome 17 open | 654434 | AF130050 |
| | | reading frame 86 | | |
| 34408_at | RTN2 | reticulon 2 | 6253 | AF004222 |
| 34726_at | CACNB3 | calcium channel, voltage- | 784 | U07139 |
| | | dependent, beta 3 | | |
| | | subunit | | |
| 41113_at | ZNF500 | zinc finger protein 500 | 26048 | AI871396 |

TABLE 13

| | | | Entrez | Representative |
|---|---|---|---|---|
| Probe Set ID | Gene Symbol | Gene Title | Gene ID | Public ID |
| 200672_x_at | SPTBN1 | spectrin, beta, non-erythrocytic 1 | 6711 | NM_003128 |
| 201917_s_at | SLC25A36 | solute carrier family 25, member 36 | 55186 | AI694452 |
| 201952_at | ALCAM | activated leukocyte cell adhesion molecule | 214 | AA156721 |
| 202932_at | YES1 | v-yes-1 Yamaguchi sarcoma viral oncogene homolog 1 | 7525 | NM_005433 |
| 203139_at | DAPK1 | death-associated protein kinase 1 | 1612 | NM_004938 |
| 203372_s_at | SOCS2 | suppressor of cytokine signaling 2 | 8835 | AB004903 |
| 203875_at | SMARCA1 | SWI/SNF related, matrix associated, actin dependent | 6594 | NM_003069 |
| | | regulator of chromatin, subfamily a, member 1 | | |
| 204753_s_at | HLF | hepatic leukemia factor | 3131 | AI810712 |
| 204754_at | HLF | hepatic leukemia factor | 3131 | W60800 |
| 204755_x_at | HLF | hepatic leukemia factor | 3131 | M95585 |
| 205376_at | INPP4B | inositol polyphosphate-4-phosphatase, type II, 105 kDa | 8821 | NM_003866 |
| 205453_at | HOXB2 | homeobox B2 | 3212 | NM_002145 |
| 205984_at | CRHBP | corticotropin releasing hormone binding protein | 1393 | NM_001882 |
| 206099_at | PRKCH | protein kinase C, eta | 5583 | NM_006255 |
| 206310_at | SPINK2 | serine peptidase inhibitor, Kazal type 2 (acrosin-trypsin | 6691 | NM_021114 |
| | | inhibitor) | | |
| 206478_at | KIAA0125 | KIAA0125 | 9834 | NM_014792 |
| 206674_at | FLT3 | fms-related tyrosine kinase 3 | 2322 | NM_004119 |
| 206683_at | ZNF165 | zinc finger protein 165 | 7718 | NM_003447 |
| 209487_at | RBPMS | RNA binding protein with multiple splicing | 11030 | D84109 |
| 209676_at | TFPI | tissue factor pathway inhibitor (lipoprotein-associated | 7035 | J03225 |
| | | coagulation inhibitor) | | |
| 209728_at | HLA-DRB4 | major histocompatibility complex, class II, DR beta 4 | 3126 | BC005312 |
| 209994_s_at | ABCB1 /// | ATP-binding cassette, sub-family B (MDR/TAP), member 1 /// | 5243 /// | AF016535 |
| | ABCB4 | ATP-binding cassette, sub-family B (MDR/TAP), member 4 | 5244 | |
| 210664_s_at | TFPI | tissue factor pathway inhibitor (lipoprotein-associated | 7035 | AF021834 |
| | | coagulation inhibitor) | | |
| 210665_at | TFPI | tissue factor pathway inhibitor (lipoprotein-associated | 7035 | AF021834 |
| | | coagulation inhibitor) | | |
| 212071_s_at | SPTBN1 | spectrin, beta, non-erythrocytic 1 | 6711 | BE968833 |
| 212750_at | PPP1R16B | protein phosphatase 1, regulatory (inhibitor) subunit 16B | 26051 | AB020630 |
| 213056_at | FRMD4B | FERM domain containing 4B | 23150 | AU145019 |
| 213094_at | GPR126 | G protein-coupled receptor 126 | 57211 | AL033377 |
| 213714_at | CACNB2 | calcium channel, voltage-dependent, beta 2 subunit | 783 | AI040163 |
| 213844_at | HOXA5 | homeobox A5 | 3202 | NM_019102 |
| 215388_s_at | CFH /// | complement factor H /// complement factor H-related 1 | 3075 /// | X56210 |
| | CFHR1 | | 3078 | |
| 217975_at | WBP5 | WW domain binding protein 5 | 51186 | NM_016303 |
| 218627_at | DRAM1 | DNA-damage regulated autophagy modulator 1 | 55332 | NM_018370 |
| 218764_at | PRKCH | protein kinase C, eta | 5583 | NM_024064 |
| 218772_x_at | TMEM38B | transmembrane protein 38B | 55151 | NM_018112 |
| 218901_at | PLSCR4 | phospholipid scramblase 4 | 57088 | NM_020353 |
| 218966_at | MYO5C | myosin VC | 55930 | NM_018728 |
| 219497_s_at | BCL11A | B-cell CLL/lymphoma 11A (zinc finger protein) | 53335 | NM_022893 |
| 221458_at | HTR1F | 5-hydroxytryptamine (serotonin) receptor 1F | 3355 | NM_000866 |
| 221773_at | ELK3 | ELK3, ETS-domain protein (SRF accessory protein 2) | 2004 | AW575374 |
| 221942_s_at | GUCY1A3 | guanylate cyclase 1, soluble, alpha 3 | 2982 | AI719730 |
| 41577_at | PPP1R16B | protein phosphatase 1, regulatory (inhibitor) subunit 16B | 26051 | AB020630 |
| 222735_at | TMEM38B | transmembrane protein 38B | 55151 | AW452608 |
| 226547_at | MYST3 | MYST histone acetyltransferase (monocytic leukemia) 3 | 7994 | AI817830 |
| 228904_at | HOXB3 | homeobox B3 | 3213 | AW510657 |
| 235199_at | RNF125 | ring finger protein 125 | 54941 | AI969697 |

TABLE 14
HSC-R FDR = 0.05 Probe List

| | Gene | | Entrez | Representative |
|---|---|---|---|---|
| Probe Set ID | Symbol | Gene Title | Gene ID | Public ID |
| 200033_at | DDX5 | DEAD (Asp-Glu-Ala-Asp) box | 1655 | NM_004396 |
| | | polypeptide 5 | | |
| 200672_x_at | SPTBN1 | spectrin, beta, non-erythrocytic 1 | 6711 | NM_003128 |
| 200962_at | RPL31 | ribosomal protein L31 | 6160 | AI348010 |
| 201466_s_at | JUN | jun oncogene | 3725 | NM_002228 |
| 201625_s_at | INSIG1 | insulin induced gene 1 | 3638 | BE300521 |
| 201695_s_at | PNP | purine nucleoside phosphorylase | 4860 | NM_000270 |
| 201889_at | FAM3C | family with sequence similarity 3, | 10447 | NM_014888 |
| | | member C | | |
| 201917_s_at | SLC25A36 | solute carrier family 25, member | 55186 | AI694452 |
| | | 36 | | |
| 201952_at | ALCAM | activated leukocyte cell adhesion | 214 | AA156721 |
| | | molecule | | |
| 202551_s_at | CRIM1 | cysteine rich transmembrane BMP | 51232 | BG546884 |
| | | regulator 1 (chordin-like) | | |
| 202724_s_at | FOXO1 | forkhead box O1 | 2308 | NM_002015 |
| 202822_at | LPP | LIM domain containing preferred | 4026 | BF221852 |
| | | translocation partner in lipoma | | |
| 202842_s_at | DNAJB9 | DnaJ (Hsp40) homolog, subfamily | 4189 | AL080081 |
| | | B, member 9 | | |
| 202932_at | YES1 | v-yes-1 Yamaguchi sarcoma viral | 7525 | NM_005433 |
| | | oncogene homolog 1 | | |
| 203139_at | DAPK1 | death-associated protein kinase 1 | 1612 | NM_004938 |
| 203372_s_at | SOCS2 | suppressor of cytokine signaling 2 | 8835 | AB004903 |
| 203394_s_at | HES1 | hairy and enhancer of split 1, | 3280 | BE973687 |
| | | (Drosophila) | | |
| 203875_at | SMARCA1 | SWI/SNF related, matrix | 6594 | NM_003069 |
| | | associated, actin dependent | | |
| | | regulator of chromatin, subfamily | | |
| | | a, member 1 | | |
| 204069_at | MEIS1 | Meis homeobox 1 | 4211 | NM_002398 |
| 204304_s_at | PROM1 | prominin 1 | 8842 | NM_006017 |
| 204753_s_at | HLF | hepatic leukemia factor | 3131 | AI810712 |
| 204754_at | HLF | hepatic leukemia factor | 3131 | W60800 |
| 204755_x_at | HLF | hepatic leukemia factor | 3131 | M95585 |
| 204917_s_at | MLLT3 | myeloid/lymphoid or mixed- | 4300 | AV756536 |
| | | lineage leukemia (trithorax | | |
| | | homolog, Drosophila); | | |
| | | translocated to, 3 | | |
| 205376_at | INPP4B | inositol polyphosphate-4- | 8821 | NM_003866 |
| | | phosphatase, type II, 105 kDa | | |
| 205453_at | HOXB2 | homeobox B2 | 3212 | NM_002145 |
| 205501_at | PDE10A | phosphodiesterase 10A | 10846 | AI143879 |
| 205984_at | CRHBP | corticotropin releasing hormone | 1393 | NM_001882 |
| | | binding protein | | |
| 206099_at | PRKCH | protein kinase C, eta | 5583 | NM_006255 |
| 206310_at | SPINK2 | serine peptidase inhibitor, Kazal | 6691 | NM_021114 |
| | | type 2 (acrosin-trypsin inhibitor) | | |
| 206385_s_at | ANK3 | ankyrin 3, node of Ranvier (ankyrin | 288 | NM_020987 |
| | | G) | | |
| 206478_at | KIAA0125 | KIAA0125 | 9834 | NM_014792 |
| 206674_at | FLT3 | fms-related tyrosine kinase 3 | 2322 | NM_004119 |
| 206683_at | ZNF165 | zinc finger protein 165 | 7718 | NM_003447 |
| 207563_s_at | OGT | O-linked N-acetylglucosamine | 8473 | U77413 |
| | | (GlcNAc) transferase (UDP-N- | | |
| | | acetylglucosamine:polypeptide-N- | | |
| | | acetylglucosaminyl transferase) | | |
| 207564_x_at | OGT | O-linked N-acetylglucosamine | 8473 | NM_003605 |
| | | (GlcNAc) transferase (UDP-N- | | |
| | | acetylglucosamine:polypeptide-N- | | |
| | | acetylglucosaminyl transferase) | | |
| 208523_x_at | HIST1H2BI | histone cluster 1, H2bi | 8346 | NM_003525 |
| 208527_x_at | HIST1H2BE | histone cluster 1, H2be | 8344 | NM_003523 |
| 208707_at | EIF5 | eukaryotic translation initiation | 1983 | BE552334 |
| | | factor 5 | | |
| 208820_at | PTK2 | PTK2 protein tyrosine kinase 2 | 5747 | AL037339 |
| 208891_at | DUSP6 | dual specificity phosphatase 6 | 1848 | BC003143 |
| 208892_s_at | DUSP6 | dual specificity phosphatase 6 | 1848 | BC003143 |
| 208988_at | KDM2A | lysine (K)-specific demethylase 2A | 22992 | BE675843 |
| 209020_at | C20orf111 | chromosome 20 open reading | 51526 | AF217514 |
| | | frame 111 | | |
| 209146_at | SC4MOL | sterol-C4-methyl oxidase-like | 6307 | AV704962 |
| 209487_at | RBPMS | RNA binding protein with multiple | 11030 | D84109 |
| | | splicing | | |
| 209560_s_at | DLK1 | delta-like 1 homolog (Drosophila) | 8788 | U15979 |
| 209676_at | TFPI | tissue factor pathway inhibitor | 7035 | J03225 |
| | | (lipoprotein-associated | | |
| | | coagulation inhibitor) | | |
| 209728_at | HLA- | major histocompatibility complex, | 3126 | BC005312 |
| | DRB4 | class II, DR beta 4 | | |
| 209907_s_at | ITSN2 | intersectin 2 | 50618 | AF182198 |
| 209911_x_at | HIST1H2BD | histone cluster 1, H2bd | 3017 | BC002842 |
| 209993_at | ABCB1 | ATP-binding cassette, sub-family B | 5243 | AF016535 |
| | | (MDR/TAP), member 1 | | |
| 209994_s_at | ABCB1 | ATP-binding cassette, sub-family B | 5243 /// | AF016535 |
| | /// | (MDR/TAP), member 1 /// ATP- | 5244 | |
| | ABCB4 | binding cassette, sub-family B | | |
| | | (MDR/TAP), member 4 | | |
| 210664_s_at | TFPI | tissue factor pathway inhibitor | 7035 | AF021834 |
| | | (lipoprotein-associated | | |
| | | coagulation inhibitor) | | |
| 210665_at | TFPI | tissue factor pathway inhibitor | 7035 | AF021834 |
| | | (lipoprotein-associated | | |
| | | coagulation inhibitor) | | |
| 210942_s_at | ST3GAL6 | ST3 beta-galactoside alpha-2,3- | 10402 | AB022918 |
| | | sialyltransferase 6 | | |
| 211597_s_at | HOPX | HOP homeobox | 84525 | AB059408 |
| 212071_s_at | SPTBN1 | spectrin, beta, non-erythrocytic 1 | 6711 | BE968833 |
| 212176_at | SFRS18 | splicing factor, arginine/serine-rich | 25957 | AA902326 |
| | | 18 | | |
| 212179_at | SFRS18 | splicing factor, arginine/serine-rich | 25957 | AW157501 |
| | | 18 | | |
| 212314_at | SEL1L3 | sel-1 suppressor of lin-12-like 3 (C. elegans) | 23231 | AB018289 |
| 212488_at | COL5A1 | collagen, type V, alpha 1 | 1289 | N30339 |
| 212750_at | PPP1R16B | protein phosphatase 1, regulatory | 26051 | AB020630 |
| | | (inhibitor) subunit 16B | | |
| 212764_at | ZEB1 | zinc finger E-box binding | 6935 | AI806174 |
| | | homeobox 1 | | |
| 212958_x_at | PAM | peptidylglycine alpha-amidating | 5066 | AI022882 |
| | | monooxygenase | | |
| 213056_at | FRMD4B | FERM domain containing 4B | 23150 | AU145019 |
| 213094_at | GPR126 | G protein-coupled receptor 126 | 57211 | AL033377 |
| 213355_at | ST3GAL6 | ST3 beta-galactoside alpha-2,3- | 10402 | AI989567 |
| | | sialyltransferase 6 | | |
| 213510_x_at | LOC220594 | TL132 protein | 220594 | AW194543 |
| 213541_s_at | ERG | v-ets erythroblastosis virus E26 | 2078 | AI351043 |
| | | oncogene homolog (avian) | | |
| 213714_at | CACNB2 | calcium channel, voltage- | 783 | AI040163 |
| | | dependent, beta 2 subunit | | |
| 213750_at | RSL1D1 | ribosomal L1 domain containing 1 | 26156 | AA928506 |
| 213844_at | HOXA5 | homeobox A5 | 3202 | NM_019102 |
| 214327_x_at | TPT1 | tumor protein, translationally- | 7178 | AI888178 |
| | | controlled 1 | | |
| 214349_at | — | — | — | AV764378 |
| 215388_s_at | CFH /// | complement factor H /// | 3075 /// | X56210 |
| | CFHR1 | complement factor H-related 1 | 3078 | |
| 215779_s_at | HIST1H2BG | histone cluster 1, H2bg | 8339 | BE271470 |
| 217975_at | WBP5 | WW domain binding protein 5 | 51186 | NM_016303 |
| 218280_x_at | HIST2H2AA3 | histone cluster 2, H2aa3 /// | 723790 | NM_003516 |
| | /// | histone cluster 2, H2aa4 | /// 8337 | |
| | HIST2H2AA4 | | | |
| 218332_at | BEX1 | brain expressed, X-linked 1 | 55859 | NM_018476 |
| 218379_at | RBM7 | RNA binding motif protein 7 | 10179 | NM_016090 |
| 218627_at | DRAM1 | DNA-damage regulated autophagy | 55332 | NM_018370 |
| | | modulator 1 | | |
| 218723_s_at | C13orf15 | chromosome 13 open reading | 28984 | NM_014059 |
| | | frame 15 | | |
| 218764_at | PRKCH | protein kinase C, eta | 5583 | NM_024064 |
| 218772_x_at | TMEM38B | transmembrane protein 38B | 55151 | NM_018112 |
| 218899_s_at | BAALC | brain and acute leukemia, | 79870 | NM_024812 |
| | | cytoplasmic | | |
| 218901_at | PLSCR4 | phospholipid scramblase 4 | 57088 | NM_020353 |
| 218966_at | MYO5C | myosin VC | 55930 | NM_018728 |
| 218971_s_at | WDR91 | WD repeat domain 91 | 29062 | NM_014149 |
| 219054_at | C5orf23 | chromosome 5 open reading | 79614 | NM_024563 |
| | | frame 23 | | |
| 219497_s_at | BCL11A | B-cell CLL/lymphoma 11A (zinc | 53335 | NM_022893 |
| | | finger protein) | | |
| 219559_at | SLC17A9 | solute carrier family 17, member 9 | 63910 | NM_022082 |
| 219648_at | MREG | melanoregulin | 55686 | NM_018000 |
| 220122_at | MCTP1 | multiple C2 domains, | 79772 | NM_024717 |
| | | transmembrane 1 | | |
| 220416_at | ATP8B4 | ATPase, class I, type 8B, member 4 | 79895 | NM_024837 |
| 221458_at | HTR1F | 5-hydroxytryptamine (serotonin) | 3355 | NM_000866 |
| | | receptor 1F | | |
| 221773_at | ELK3 | ELK3, ETS-domain protein (SRF | 2004 | AW575374 |
| | | accessory protein 2) | | |
| 221833_at | LONP2 | Lon peptidase 2, peroxisomal | 83752 | AI971258 |
| 221841_s_at | KLF4 | Kruppel-like factor 4 (gut) | 9314 | BF514079 |
| 221942_s_at | GUCY1A3 | guanylate cyclase 1, soluble, alpha 3 | 2982 | AI719730 |
| 222067_x_at | HIST1H2BD | histone cluster 1, H2bd | 3017 | AL353759 |
| 222164_at | FGFR1 | fibroblast growth factor receptor 1 | 2260 | AU145411 |
| 222315_at | — | — | — | AW972855 |
| 41577_at | PPP1R16B | protein phosphatase 1, regulatory | 26051 | AB020630 |
| | | (inhibitor) subunit 16B | | |
| 60084_at | CYLD | cylindromatosis (turban tumor | 1540 | AI453099 |
| | | syndrome) | | |
| 200033_at | DDX5 | DEAD (Asp-Glu-Ala-Asp) box | 1655 | NM_004396 |
| | | polypeptide 5 | | |
| 222735_at | TMEM38B | transmembrane protein 38B | 55151 | AW452608 |
| 222815_at | RLIM | ring finger protein, LIM domain | 51132 | BE966018 |
| | | interacting | | |
| 225629_s_at | ZBTB4 | zinc finger and BTB domain | 57659 | AI669498 |
| | | containing 4 | | |
| 226206_at | MAFK | v-maf musculoaponeurotic | 7975 | BG231691 |
| | | fibrosarcoma oncogene homolog K | | |
| | | (avian) | | |
| 226420_at | MECOM | MDS1 and EVI1 complex locus | 2122 | BG261252 |
| 226545_at | CD109 | CD109 molecule | 135228 | AL110152 |
| 226547_at | MYST3 | MYST histone acetyltransferase | 7994 | AI817830 |
| | | (monocytic leukemia) 3 | | |
| 226985_at | FGD5 | FYVE, RhoGEF and PH domain | 152273 | AW269340 |
| | | containing 5 | | |
| 228465_at | — | — | — | T79942 |
| 228570_at | BTBD11 | BTB (POZ) domain containing 11 | 121551 | BF510581 |
| 228857_at | GNL1 | guanine nucleotide binding | 2794 | AA775731 |
| | | protein-like 1 | | |
| 228904_at | HOXB3 | homeobox B3 | 3213 | AW510657 |
| 228915_at | DACH1 | dachshund homolog 1 | 1602 | AI650353 |
| | | (Drosophila) | | |
| 229287_at | PCNX | pecanex homolog (Drosophila) | 22990 | BE326214 |
| 229344_x_at | RIMKLB | ribosomal modification protein | 57494 | AW135012 |
| | | rimK-like family member B | | |
| 230389_at | FNBP1 | formin binding protein 1 | 23048 | BE046511 |
| 230698_at | CALN1 | calneuron 1 | 83698 | AW072102 |
| 230788_at | GCNT2 | glucosaminyl (N-acetyl) | 2651 | BF059748 |
| | | transferase 2, I-branching enzyme | | |
| | | (I blood group) | | |
| 232098_at | DST | dystonin | 667 | AK025142 |
| 232231_at | RUNX2 | runt-related transcription factor 2 | 860 | AL353944 |
| 234994_at | TMEM200A | transmembrane protein 200A | 114801 | AA088177 |
| 235048_at | FAM169A | family with sequence similarity | 26049 | AV720650 |
| | | 169, member A | | |
| 235199_at | RNF125 | ring finger protein 125 | 54941 | AI969697 |
| 235252_at | KSR1 | kinase suppressor of ras 1 | 8844 | AI090141 |
| 235490_at | TMEM107 | transmembrane protein 107 | 84314 | AV743951 |
| 235826_at | — | — | — | AI693281 |
| 236193_at | HIST1H2BC | histone cluster 1, H2bc | 8347 | AA037483 |
| 238041_at | TCF12 | transcription factor 12 | 6938 | AA151712 |
| 238488_at | IPO11 | importin 11 /// leucine rich repeat | 100130733 | BF511602 |
| | /// | containing 70 | /// | |
| | LRRC70 | | 51194 | |
| 238633_at | — | — | — | W93523 |
| 238974_at | C2orf69 | chromosome 2 open reading | 205327 | N47077 |
| | | frame 69 | | |
| 239328_at | — | — | — | AW512339 |
| 239451_at | — | — | — | AI684643 |
| 239835_at | KBTBD8 | kelch repeat and BTB (POZ) | 84541 | AA669114 |
| | | domain containing 8 | | |
| 240165_at | — | — | — | AI678013 |
| 241756_at | — | — | — | T51136 |
| 243010_at | MSI2 | musashi homolog 2 (Drosophila) | 124540 | BE000929 |
| 243092_at | LOC100288730 | hypothetical LOC100288730 | 100288730 | AI140189 |
| 243835_at | ZDHHC21 | zinc finger, DHHC-type containing | 340481 | BE467787 |
| | | 21 | | |
| 244110_at | MLL | Myeloid/lymphoid or mixed- | 4297 | BE669782 |
| | | lineage leukemia (trithorax | | |
| | | homolog, Drosophila) | | |
| 244447_at | — | — | — | AW292830 |
| 244519_at | ASXL1 | additional sex combs like 1 | 171023 | AI829840 |
| | | (Drosophila) | | |

TABLE 15
Summary of 7 additional patient samples used in generation
of CD34+/CD38− vs CD34+/CD38+ signature

| | Relapse or | | | | |
|---|---|---|---|---|---|
| AML # | Diagnosis | FAB | Age | Sex | Karyotype |
| 17 | Diag | M2 | 83 | m | 49xy, +3, +9, +12 |
| 18 | No data | M4 | No data | No data | No data |
| 19 | Diag | M5b | 47 | m | 8 |
| 20 | Diag | M0 | 70 | f | complex |
| 21 | Relapse | M6 | 48 | f | normal |
| 22 | Diag | M4 | 65 | f | complex |
| 23 | Diag | M4 | 63 | f | normal |

TABLE 16
Additive Correlation of LSC-R Probes and Patient Outcome

| | | p value - |
|---|---|---|
| Rank in LSC-R | | correlation with |
| gene list | LSC probeID | overall survival |
| 1 | 220128_s_at | 0.977682866 |
| 2 | 209488_s_at | 0.194703224 |
| 3 | 215411_s_at | 0.075914897 |
| 4 | 201702_s_at | 0.138735935 |
| 5 | 201243_s_at | 0.003708531 |
| 6 | 212676_at | 7.47E−05 |
| 7 | 209487_at | 7.14E−05 |
| 8 | 219871_at | 0.000175004 |
| 9 | 207836_s_at | 0.000151178 |
| 10 | 211113_s_at | 0.000151178 |
| 11 | 214252_s_at | 5.63E−06 |
| 12 | 212976_at | 4.95E−05 |
| 13 | 213056_at | 6.23E−05 |
| 14 | 207090_x_at | 6.23E−05 |
| 15 | 221621_at | 9.59E−05 |
| 16 | 218183_at | 7.56E−05 |
| 17 | 216262_s_at | 5.89E−05 |
| 18 | 204028_s_at | 2.26E−05 |
| 19 | 208993_s_at | 0.000107232 |
| 20 | 206582_s_at | 0.000122344 |
| 21 | 205321_at | 1.98E−05 |
| 22 | 209272_at | 2.76E−06 |
| 23 | 218907_s_at | 8.89E−06 |
| 24 | 201242_s_at | 8.25E−06 |
| 25 | 41113_at | 8.57E−07 |
| 26 | 202646_s_at | 8.57E−07 |
| 27 | 215557_at | 8.57E−07 |
| 28 | 212875_s_at | 7.94E−06 |
| 29 | 219236_at | 2.90E−06 |
| 30 | 213861_s_at | 4.39E−06 |
| 31 | 221483_s_at | 7.23E−07 |
| 32 | 214197_s_at | 4.05E−06 |
| 33 | 205256_at | 1.52E−06 |
| 34 | 207837_at | 4.90E−07 |
| 35 | 214860_at | 4.53E−07 |
| 36 | 211537_x_at | 1.11E−06 |
| 37 | 213433_at | 1.11E−06 |
| 38 | 207753_at | 2.86E−06 |
| 39 | 212114_at | 8.40E−06 |
| 40 | 214004_s_at | 1.73E−05 |
| 41 | 208883_at | 2.12E−06 |
| 42 | 219658_at | 2.12E−06 |
| 43 | 213557_at | 2.12E−06 |
| 44 | 203474_at | 1.02E−05 |
| 45 | 214745_at | 1.02E−05 |
| 46 | 202956_at | 1.54E−06 |
| 47 | 211536_x_at | 1.54E−06 |
| 48 | 209740_s_at | 5.21E−06 |
| 49 | 34408_at | 1.66E−05 |
| 50 | 201018_at | 1.54E−06 |
| 51 | 214820_at | 1.83E−06 |
| 52 | 212397_at | 8.92E−07 |
| 53 | 221294_at | 8.92E−07 |
| 54 | 219718_at | 1.43E−06 |
| 55 | 209425_at | 8.24E−07 |
| 56 | 220360_at | 1.43E−06 |
| 57 | 213313_at | 1.23E−06 |
| 58 | 204837_at | 9.79E−07 |
| 59 | 205094_at | 9.79E−07 |
| 60 | 211877_s_at | 6.40E−06 |
| 61 | 205702_at | 2.77E−05 |
| 62 | 212851_at | 5.69E−05 |
| 63 | 206874_s_at | 1.56E−05 |
| 64 | 219232_s_at | 1.56E−05 |
| 65 | 201080_at | 6.72E−05 |
| 66 | 209200_at | 0.000222547 |
| 67 | 208634_s_at | 0.001135991 |
| 68 | 205608_s_at | 0.002585716 |
| 69 | 214738_s_at | 0.002585716 |
| 70 | 207968_s_at | 0.002585716 |
| 71 | 203106_s_at | 0.002585716 |
| 72 | 213639_s_at | 0.002585716 |
| 73 | 202599_s_at | 0.0008893 |
| 74 | 211255_x_at | 0.000836413 |
| 75 | 219383_at | 0.000780115 |
| 76 | 207737_at | 0.000615825 |
| 77 | 221020_s_at | 0.000836413 |
| 78 | 206945_at | 0.00057802 |
| 79 | 34726_at | 0.000103453 |
| 80 | 210132_at | 0.000103453 |
| 81 | 206061_s_at | 0.00057802 |
| 82 | 212299_at | 0.000992242 |
| 83 | 204592_at | 0.000992242 |
| 84 | 209814_at | 0.000714641 |
| 85 | 202629_at | 0.000808658 |
| 86 | 205762_s_at | 0.000808658 |
| 87 | 202817_s_at | 0.000808658 |
| 88 | 218724_s_at | 0.000714641 |
| 89 | 204217_s_at | 0.00037038 |
| 90 | 219603_s_at | 0.000808658 |
| 91 | 210694_s_at | 0.000325071 |
| 92 | 212276_at | 0.000325071 |
| 93 | 212678_at | 0.000325071 |
| 94 | 207034_s_at | 0.000191313 |
| 95 | 209199_s_at | 0.002994201 |
| 96 | 208879_x_at | 0.00038804 |
| 97 | 206822_s_at | 0.00038804 |
| 98 | 212796_s_at | 0.00036057 |
| 99 | 213322_at | 0.000852069 |
| 100 | 213666_at | 0.000504441 |

TABLE 17

| | | |
|---|---|---|
| Probe Set Name | Probe Sequence | Sequence ID No: |

| | | |
|---|---|---|
| 200033_at | AGAATGGTGTTTACAGTGCTGCAAA | SEQ ID NO: 1374 |
| 200033_at | CAGTGCTGCAAATTACACCAATGGG | SEQ ID NO: 1375 |
| 200033_at | GAAGTAATTTTGTGTCTGCTGGTAT | SEQ ID NO: 1376 |
| 200033_at | AGGACTGGTAATCCAACAGGGACTT | SEQ ID NO: 1377 |
| 200033_at | GAATGGTTATGATAGCACTCAGCAA | SEQ ID NO: 1378 |
| 200033_at | TGCATATCCTGCTACTGCAGCTGCA | SEQ ID NO: 1379 |
| 200033_at | GCAGCTGCACCTATGATTGGTTATC | SEQ ID NO: 1380 |
| 200033_at | TCCAATGCCAACAGGATATTCCCAA | SEQ ID NO: 1381 |
| 200033_at | GTCTGTTTTTCATAATTGCTCTTTA | SEQ ID NO: 1382 |
| 200033_at | TATGGTGCACTTTTTCGCTATTTAA | SEQ ID NO: 1383 |
| 200033_at | AGTTGGATATTTCTCTACATTCCTG | SEQ ID NO: 1384 |
| 200033_at | AGAATGGTGTTTACAGTGCTGCAAA | SEQ ID NO: 1385 |
| 200033_at | CAGTGCTGCAAATTACACCAATGGG | SEQ ID NO: 1386 |
| 200033_at | GAAGTAATTTTGTGTCTGCTGGTAT | SEQ ID NO: 1387 |
| 200033_at | AGGACTGGTAATCCAACAGGGACTT | SEQ ID NO: 1388 |
| 200033_at | GAATGGTTATGATAGCACTCAGCAA | SEQ ID NO: 1389 |
| 200033_at | TGCATATCCTGCTACTGCAGCTGCA | SEQ ID NO: 1390 |
| 200033_at | GCAGCTGCACCTATGATTGGTTATC | SEQ ID NO: 1391 |
| 200033_at | TCCAATGCCAACAGGATATTCCCAA | SEQ ID NO: 1392 |
| 200033_at | GTCTGTTTTTCATAATTGCTCTTTA | SEQ ID NO: 1393 |
| 200033_at | TATGGTGCACTTTTTCGCTATTTAA | SEQ ID NO: 1394 |
| 200033_at | AGTTGGATATTTCTCTACATTCCTG | SEQ ID NO: 1395 |
| 200962_at | GATATGAGTCTGCATGGCCTCAGGA | SEQ ID NO: 1396 |
| 200962_at | GATTTTAGGTTGTCTGCACTCTAGC | SEQ ID NO: 1397 |
| 200962_at | GCACTCTAGCTTTTTTGTCGTTTTC | SEQ ID NO: 1398 |
| 200962_at | ATACATCATATCTTAATTTCCACTG | SEQ ID NO: 1399 |
| 200962_at | TCTACACGGCCGGGGTTTCAACAAG | SEQ ID NO: 1400 |
| 200962_at | ACAAGGTACTGATGTCTTCTGCCCT | SEQ ID NO: 1401 |
| 200962_at | TGCCCTTGCCTCTTCGACAGGCAAG | SEQ ID NO: 1402 |
| 200962_at | ATTCTTTAGGCACACAAATTCACAT | SEQ ID NO: 1403 |
| 200962_at | CATTATACTTCCTGATCTGTGATTG | SEQ ID NO: 1404 |
| 200962_at | TCGTAACTAGTATGTCTGTCCCACC | SEQ ID NO: 1405 |
| 200962_at | TATGTCTGTCCCACCTTTAAAAAGT | SEQ ID NO: 1406 |
| 201466_s_at | AAATCACTCTCAGTGCTTCTTACTA | SEQ ID NO: 1407 |
| 201466_s_at | GCAGTAAAAACTGTTCTCTATTAGA | SEQ ID NO: 1408 |
| 201466_s_at | ATGTACCTGATGTACCTGATGCTAT | SEQ ID NO: 1409 |
| 201466_s_at | ATCTATATGGAATTGCTTACCAAAG | SEQ ID NO: 1410 |
| 201466_s_at | TAGTGCGATGTTTCAGGAGGCTGGA | SEQ ID NO: 1411 |
| 201466_s_at | AGCCCACTGAGAAGTCAAACATTTC | SEQ ID NO: 1412 |
| 201466_s_at | GTGGCATGTGCTGTGACCATTTATA | SEQ ID NO: 1413 |
| 201466_s_at | TTTACAATAGGTGCTTATTCTCAAA | SEQ ID NO: 1414 |
| 201466_s_at | AGGTGCTTATTCTCAAAGCAGGAAT | SEQ ID NO: 1415 |
| 201466_s_at | GCAGGAATTGGTGGCAGATTTTACA | SEQ ID NO: 1416 |
| 201466_s_at | CTTCTCTTTGACAATTCCTAGATAA | SEQ ID NO: 1417 |
| 201625_s_at | TAGCAGCCCTATCTTTGGGCCTTTG | SEQ ID NO: 1418 |
| 201625_s_at | GGGCCTTTGGTGGACATTTGATCGT | SEQ ID NO: 1419 |
| 201625_s_at | TGATCGTTCCAGAAGTGGCCTTGGG | SEQ ID NO: 1420 |
| 201625_s_at | GGCTGGGGATCACCATAGCTTTTCT | SEQ ID NO: 1421 |
| 201625_s_at | TAGCTACGCTGATCACGCAGTTTCT | SEQ ID NO: 1422 |
| 201625_s_at | TTCCTCTATATTCGTTCTTGGCTCC | SEQ ID NO: 1423 |
| 201625_s_at | TTTTCTCAGGAGGCGTCACGGTGGG | SEQ ID NO: 1424 |
| 201625_s_at | GTTCCTGAAAAGCCCCATAGTGATT | SEQ ID NO: 1425 |
| 201625_s_at | GGGCTGACTGTACAAATGACTCCTG | SEQ ID NO: 1426 |
| 201625_s_at | GATGACTTACCCTGAAGTCTTCCCT | SEQ ID NO: 1427 |
| 201625_s_at | CTTCCCAAGTATTCGATTTCATTCA | SEQ ID NO: 1428 |
| 201695_s_at | TCCCACACAAGACCCAAGTAGCTGC | SEQ ID NO: 1429 |
| 201695_s_at | CCAAGTAGCTGCTACCTTCTTTGGC | SEQ ID NO: 1430 |
| 201695_s_at | TCTACCAGACCCTTCTGGTGCCAGA | SEQ ID NO: 1431 |
| 201695_s_at | TCATTCCTGTTCTTTCTTACACAAG | SEQ ID NO: 1432 |
| 201695_s_at | GACTCGGGCCTTAGAACTTTGCATA | SEQ ID NO: 1433 |
| 201695_s_at | ATAGCAGCTGCTACTAGCTCTTTGA | SEQ ID NO: 1434 |
| 201695_s_at | ATACATTCCGAGGGGCTCAGTTCTG | SEQ ID NO: 1435 |
| 201695_s_at | GCTTCTCACTCATCACTAACAAGGT | SEQ ID NO: 1436 |
| 201695_s_at | GAACAGTTTGTCTCCATTCTTATGG | SEQ ID NO: 1437 |
| 201695_s_at | TCCATTCTTATGGCCAGCATTCCAC | SEQ ID NO: 1438 |
| 201695_s_at | ACTCCCTGACAAAGCCAGTTGACCT | SEQ ID NO: 1439 |
| 201917_s_at | TTCAGACTCTATCTTTGCTTGTTCA | SEQ ID NO: 1440 |
| 201917_s_at | TGGGTCTCTTTATCGTGGTCTGACA | SEQ ID NO: 1441 |
| 201917_s_at | CGTGGTCTGACAACTCATCTAGTGA | SEQ ID NO: 1442 |
| 201917_s_at | GAATTGGTGGTTTACCTACTCAATG | SEQ ID NO: 1443 |
| 201917_s_at | GCAGCACGAGGACTGCTGTACTGCA | SEQ ID NO: 1444 |
| 201917_s_at | ATCACACCACATTACTTGGCCTTTC | SEQ ID NO: 1445 |
| 201917_s_at | GGGCATGTCTGCTTCATATGCTGGT | SEQ ID NO: 1446 |
| 201917_s_at | ATCAGAGACTGTTATCCATTTTGTT | SEQ ID NO: 1447 |
| 201917_s_at | GTGGGAATGATGCTAGCTGCTGCCA | SEQ ID NO: 1448 |
| 201917_s_at | GCTGCCACCTCAAAAACTTGTGCCA | SEQ ID NO: 1449 |
| 201917_s_at | GCCACAACTATAGCATATCCACATG | SEQ ID NO: 1450 |
| 201952_at | ACCTGCTCTCCACAATAAATCACAA | SEQ ID NO: 1451 |
| 201952_at | ACAGCTGTCAGAACCTCGAGAGCAG | SEQ ID NO: 1452 |
| 201952_at | ACTCAGAGCTCTGGACCGAAAGCAG | SEQ ID NO: 1453 |
| 201952_at | ATTACCATCGATTCAGTGCCTGGAT | SEQ ID NO: 1454 |
| 201952_at | GCTTACTTGTTTAATGGCAGCCACA | SEQ ID NO: 1455 |
| 201952_at | GGCAGCCACATGCACGAAGATGCTA | SEQ ID NO: 1456 |
| 201952_at | GAATTCCAAATCCTCAACTTTTGAG | SEQ ID NO: 1457 |
| 201952_at | ACTTTTGAGGTTTCGGCTCTCCAAT | SEQ ID NO: 1458 |
| 201952_at | TTCGGCTCTCCAATTTAACTCTTTG | SEQ ID NO: 1459 |
| 201952_at | AGTTCAAGGTTCACTCCCTATATGT | SEQ ID NO: 1460 |
| 201952_at | GATTAACATACCCGTCTATGCCTAA | SEQ ID NO: 1461 |
| 202724_s_at | GAGCAGTAAATCAATGGAACATCCC | SEQ ID NO: 1462 |
| 202724_s_at | ACAAATTGGACTTGTTCAACTGCTG | SEQ ID NO: 1463 |
| 202724_s_at | CAGCCCCAACTTAAAATTCTTACAT | SEQ ID NO: 1464 |
| 202724_s_at | ACAGACCAACCTGGCATTACAGTTG | SEQ ID NO: 1465 |
| 202724_s_at | TTGGCCTCTCCTTGAGGTGGGCACA | SEQ ID NO: 1466 |
| 202724_s_at | GCCAGGGGTGGCCATGTAAGTCCCA | SEQ ID NO: 1467 |
| 202724_s_at | GCTACCCGAGTTTAGTAACAGTGCA | SEQ ID NO: 1468 |
| 202724_s_at | AACAGTGCAGATTCCACGTTCTTGT | SEQ ID NO: 1469 |
| 202724_s_at | CGTTCTTGTTCCGATACTCTGAGAA | SEQ ID NO: 1470 |
| 202724_s_at | GATGTTGATGTACTTACAGACACAA | SEQ ID NO: 1471 |
| 202724_s_at | GACACAAGAACAATCTTTGCTATAA | SEQ ID NO: 1472 |
| 202822_at | CTCTTGTCAAATCTGTGTCGGCTGC | SEQ ID NO: 1473 |
| 202822_at | CCCTGATCCTTCCATTATCAAGTTT | SEQ ID NO: 1474 |
| 202822_at | ACTGATGTAACCTCAAAGCCTCTCA | SEQ ID NO: 1475 |
| 202822_at | TCACCATTCCTCTTGGCTTGGAAAG | SEQ ID NO: 1476 |
| 202822_at | ACAAGCGATTGTCCATCTGTTGCCT | SEQ ID NO: 1477 |
| 202822_at | CCTGCTTTAGCCATCTGTGGGAAAC | SEQ ID NO: 1478 |
| 202822_at | GACACCTCTGCAAAATGTGCCTCAA | SEQ ID NO: 1479 |
| 202822_at | GTGCCTCAAGTCCATTTCTTGGGAT | SEQ ID NO: 1480 |
| 202822_at | CATTTCTTGGGATCGCTCGTTTGGT | SEQ ID NO: 1481 |
| 202822_at | GTTTGGTGCACTCTCGTGGGAGACA | SEQ ID NO: 1482 |
| 202822_at | AACATATACTTGTGCCTTATTTTCA | SEQ ID NO: 1483 |
| 202842_s_at | GTTTGATATTTACCACAGCGCTGTG | SEQ ID NO: 1484 |
| 202842_s_at | GCGCTGTGCCTTTCTACAGTAGAAC | SEQ ID NO: 1485 |
| 202842_s_at | GGTTTTATTGCCCATAGTCATTTAG | SEQ ID NO: 1486 |
| 202842_s_at | ATATTTCTTTCTTAGTTGTTGGCAC | SEQ ID NO: 1487 |
| 202842_s_at | GTTGTTGGCACTCTTAGGTCTTAGT | SEQ ID NO: 1488 |
| 202842_s_at | GTGTGTGTGTAGTTTATCCTCTCTC | SEQ ID NO: 1489 |
| 202842_s_at | GATTGACTGATACCTCATTCTGTTT | SEQ ID NO: 1490 |
| 202842_s_at | AATTTCTGTGCAACCTTACTATGTG | SEQ ID NO: 1491 |
| 202842_s_at | GTGTGCTTTTGTTTTCGGATAGACT | SEQ ID NO: 1492 |
| 202842_s_at | ATTTCTTTAGTTCTGCACTTTTCCA | SEQ ID NO: 1493 |
| 202842_s_at | CACTTTTCCACATTATACTCCATAT | SEQ ID NO: 1494 |
| 202932_at | TGGCAGTGGTTCTGGTACTAAAAAT | SEQ ID NO: 1495 |
| 202932_at | GTTCTGGTACTAAAAATTGTGGTTG | SEQ ID NO: 1496 |
| 202932_at | TTTTCTGTTTACGTAACCTGCTTAG | SEQ ID NO: 1497 |
| 202932_at | ACGTAACCTGCTTAGTATTGACACT | SEQ ID NO: 1498 |
| 202932_at | AACCTGCTTAGTATTGACACTCTCT | SEQ ID NO: 1499 |
| 202932_at | GCTTAGTATTGACACTCTCTACCAA | SEQ ID NO: 1500 |
| 202932_at | GACACTCTCTACCAAGAGGGTCTTC | SEQ ID NO: 1501 |
| 202932_at | CTCTACCAAGAGGGTCTTCCTAAGA | SEQ ID NO: 1502 |
| 202932_at | CTTCCTAAGAAGAGTGCTGTCATTA | SEQ ID NO: 1503 |
| 202932_at | GAGTGCTGTCATTATTTCCTCTTAT | SEQ ID NO: 1504 |
| 202932_at | TTTCCTCTTATCAACAACTTGTGAC | SEQ ID NO: 1505 |
| 203372_s_at | GAGATAGCTCGCATTCAGACTACCT | SEQ ID NO: 1506 |
| 203372_s_at | CTCGCATTCAGACTACCTACTAACA | SEQ ID NO: 1507 |
| 203372_s_at | TCAGCTGGACCAACTAATCTTCGAA | SEQ ID NO: 1508 |
| 203372_s_at | AAATTCAGATTGGACTCTATCATAT | SEQ ID NO: 1509 |
| 203372_s_at | ATCATATGTGTCAAATCCAAGCTTA | SEQ ID NO: 1510 |
| 203372_s_at | GTGTGGTTCATCTGATCGACTACTA | SEQ ID NO: 1511 |
| 203372_s_at | TCGACTACTATGTTCAGATGTGCAA | SEQ ID NO: 1512 |
| 203372_s_at | TAAGCGGACAGGTCCAGAAGCCCCC | SEQ ID NO: 1513 |
| 203372_s_at | ACTGTTCACCTTTATCTGACCAAAC | SEQ ID NO: 1514 |
| 203372_s_at | CTGACCAAACCGCTCTACACGTCAG | SEQ ID NO: 1515 |
| 203372_s_at | TTAACAAATGTACCGGTGCCATCTG | SEQ ID NO: 1516 |
| 203394_s_at | AGGATCCGGAGCTGGTGCTGATAAC | SEQ ID NO: 1517 |
| 203394_s_at | TGCTGATAACAGCGGAATCCCCCGT | SEQ ID NO: 1518 |
| 203394_s_at | TTGGTCCTGGAACAGCGCTACTGAT | SEQ ID NO: 1519 |
| 203394_s_at | GTCCTGGAACAGCGCTACTGATCAC | SEQ ID NO: 1520 |
| 203394_s_at | GGAACAGCGCTACTGATCACCAAGT | SEQ ID NO: 1521 |
| 203394_s_at | TCACCAAGTAGCCACAAAATATAAT | SEQ ID NO: 1522 |
| 203394_s_at | TATAATAAACCCTCAGCACTTGCTC | SEQ ID NO: 1523 |
| 203394_s_at | ATAAACCCTCAGCACTTGCTCAGTA | SEQ ID NO: 1524 |
| 203394_s_at | AACCCTCAGCACTTGCTCAGTAGTT | SEQ ID NO: 1525 |
| 203394_s_at | CAGCACTTGCTCAGTAGTTTTGTGA | SEQ ID NO: 1526 |
| 203394_s_at | GTAGTTTTGTGAAAGTCTCAAGTAA | SEQ ID NO: 1527 |
| 203875_at | GATTTAACATTGTTGGGCCATTTAA | SEQ ID NO: 1528 |
| 203875_at | AAATGTGCATATTGGAGCAGAACAT | SEQ ID NO: 1529 |
| 203875_at | ATCTGTTTCCATTTTAGTCACAGAA | SEQ ID NO: 1530 |
| 203875_at | ACAATGCTTTCTACCTGAAATGTGT | SEQ ID NO: 1531 |
| 203875_at | CCTCTCAGTCCTTGTTCTTTTGAAG | SEQ ID NO: 1532 |
| 203875_at | GTCCTTGTTCTTTTGAAGCTTGTGC | SEQ ID NO: 1533 |
| 203875_at | GCTTGTGCTGAGGTTTTAGCTTTTC | SEQ ID NO: 1534 |
| 203875_at | GTGCTGAGGTTTTAGCTTTTCTATG | SEQ ID NO: 1535 |
| 203875_at | GCCGCTGCTTTGAAAGAGAACCTAG | SEQ ID NO: 1536 |
| 203875_at | GAGAACCTAGATTCTATAGTTGTAT | SEQ ID NO: 1537 |
| 203875_at | TATTATTGTTGTTTCATACTTTAAA | SEQ ID NO: 1538 |
| 205453_at | GGTCCCTTTTTCCGAGGAAGAGCTG | SEQ ID NO: 1539 |
| 205453_at | ATTTTTTCACCAGTACGCTCTGTGC | SEQ ID NO: 1540 |
| 205453_at | CTCCTTGGCCGTCTACTGGAAAAAT | SEQ ID NO: 1541 |
| 205453_at | ACTGGAAAAATCGAGCCTCTCCCAC | SEQ ID NO: 1542 |
| 205453_at | CCACCCTCAGTCGCATAGACTTATG | SEQ ID NO: 1543 |
| 205453_at | GAATTAGCGTTTAATCCACTTCCTT | SEQ ID NO: 1544 |
| 205453_at | TATTGGGCACTCGGTTATCTTTTAA | SEQ ID NO: 1545 |
| 205453_at | TTCCGTTTGGTAGACTCCTTCCAAT | SEQ ID NO: 1546 |
| 205453_at | GGTAGACTCCTTCCAATGAAATCTC | SEQ ID NO: 1547 |
| 205453_at | CCCGGGCCATTGCCAGAAGACGTCT | SEQ ID NO: 1548 |
| 205453_at | GCCAGAAGACGTCTTCTCGGGGCGC | SEQ ID NO: 1549 |
| 205501_at | ATGCTTGCCCAACACACTGTGAAAT | SEQ ID NO: 1550 |
| 205501_at | ATGCAGCATCTTCATTCTTTCTGAG | SEQ ID NO: 1551 |
| 205501_at | GATGGTTTTCTTTACATGAACAAAT | SEQ ID NO: 1552 |
| 205501_at | GAGATCCTAGATCCATAACGTAGCT | SEQ ID NO: 1553 |
| 205501_at | AAGGCATCTAAGAGTTTGCTGTTGA | SEQ ID NO: 1554 |
| 205501_at | TGCTGTTGATAATCTTGCTGACCAA | SEQ ID NO: 1555 |
| 205501_at | GTAACACAGGTTATATGCCATCACA | SEQ ID NO: 1556 |
| 205501_at | ATGCCATCACAAATACAATGCTCAT | SEQ ID NO: 1557 |
| 205501_at | AGAGTCAATGAACCTGTGTCCAGAA | SEQ ID NO: 1558 |
| 205501_at | AGAGGTCTTAACTTTGCATTTATAA | SEQ ID NO: 1559 |
| 205501_at | TCATTTGCAGTCTTTGTATTTAAAA | SEQ ID NO: 1560 |
| 206099_at | ATGATGAGGTGGTCTACCCTACCTG | SEQ ID NO: 1561 |
| 206099_at | CCCTACCTGGCTCCATGAAGATGCC | SEQ ID NO: 1562 |
| 206099_at | CAGGGAGGCGAGCACGCCATCTTGA | SEQ ID NO: 1563 |
| 206099_at | GCCATCTTGAGACATCCTTTTTTTA | SEQ ID NO: 1564 |
| 206099_at | TTAAGGAAATCGACTGGGCCCAGCT | SEQ ID NO: 1565 |
| 206099_at | GAACCATCGCCAAATAGAACCGCCT | SEQ ID NO: 1566 |
| 206099_at | TCAGACCCAGAATCAAATCCCGAGA | SEQ ID NO: 1567 |
| 206099_at | AGAAACTTTTCCTATGTGTCTCCAG | SEQ ID NO: 1568 |
| 206099_at | GTGTCTCCAGAATTGCAACCATAGC | SEQ ID NO: 1569 |
| 206099_at | CCAGGAATTTCCTCTATCGGACCTT | SEQ ID NO: 1570 |
| 206099_at | CTTCCCAGCATCAGCCTTAGAACAA | SEQ ID NO: 1571 |
| 206310_at | CTCTGATCCCTCAATTTGGTCTGTT | SEQ ID NO: 1572 |
| 206310_at | ATAGAACGCCAAACTGCTCTCAGTA | SEQ ID NO: 1573 |
| 206310_at | TAGATTACCAGGATGTCCCAGACAC | SEQ ID NO: 1574 |
| 206310_at | TCCCAGACACTTTAACCCTGTGTGT | SEQ ID NO: 1575 |
| 206310_at | CCCTGTGTGTGGCAGTGACATGTCC | SEQ ID NO: 1576 |
| 206310_at | GTGACATGTCCACTTATGCCAATGA | SEQ ID NO: 1577 |
| 206310_at | CATTCGAAATGGACCCTGCTGATGG | SEQ ID NO: 1578 |
| 206310_at | GGCGCAGGTAACAGACCGCAGGGGC | SEQ ID NO: 1579 |
| 206310_at | AGAATCCTTGTTTCTTGGCTTTTGC | SEQ ID NO: 1580 |
| 206310_at | TCTTGGCTTTTGCTCCTGGAGTTAA | SEQ ID NO: 1581 |
| 206310_at | GAGTTAAGCTTACTGCCCAGGTGAC | SEQ ID NO: 1582 |
| 206674_at | GATGGCCGTGTTTCGGAATGTCCTC | SEQ ID NO: 1583 |
| 206674_at | TCCTCACACCTACCAAAACAGGCGA | SEQ ID NO: 1584 |
| 206674_at | AAACAGGCGACCTTTCAGCAGAGAG | SEQ ID NO: 1585 |
| 206674_at | AGATGGATTTGGGGCTACTCTCTCC | SEQ ID NO: 1586 |
| 206674_at | CTCCGCAGGCTCAGGTCGAAGATTC | SEQ ID NO: 1587 |
| 206674_at | TAGTTTTAAGGACTTCATCCCTCCA | SEQ ID NO: 1588 |
| 206674_at | CCACCTATCCCTAACAGGCTGTAGA | SEQ ID NO: 1589 |
| 206674_at | TTATCAACTGCTGCTTCACCAGACT | SEQ ID NO: 1590 |
| 206674_at | TTTCTCTAGAAGCCGTCTGCGTTTA | SEQ ID NO: 1591 |
| 206674_at | GGAGCATTGATCTGCATCCAAGGCC | SEQ ID NO: 1592 |
| 206674_at | GGCCGGCTTGAGTGAATTGTGTACC | SEQ ID NO: 1593 |
| 207563_s_at | AGCGTGTTCCCAATAGTGTACTCTG | SEQ ID NO: 1594 |
| 207563_s_at | TACTCTGGCTGTTGCGTTTTCCAGC | SEQ ID NO: 1595 |
| 207563_s_at | GCCCCAGAACCGTATCATTTTTTCA | SEQ ID NO: 1596 |
| 207563_s_at | GAGGAACACGTCAGGAGAGGCCAGC | SEQ ID NO: 1597 |
| 207563_s_at | GGACACTCCACTCTGTAATGGGCAC | SEQ ID NO: 1598 |
| 207563_s_at | GATGGATGTCCTCTGGGCAGGGACC | SEQ ID NO: 1599 |
| 207563_s_at | ACCCCCATGGTGACTATGCCAGGAG | SEQ ID NO: 1600 |
| 207563_s_at | AGGAGAGACTCTTGCTTCTCGAGTT | SEQ ID NO: 1601 |
| 207563_s_at | ATCCCAGCTCACTTGCTTAGGTTGT | SEQ ID NO: 1602 |
| 207563_s_at | GAGCGGCTCTATCTACAGATGTGGG | SEQ ID NO: 1603 |
| 207563_s_at | TGCAGCTGGCAACAAACCTGACCAC | SEQ ID NO: 1604 |
| 207564_x_at | TCAGTCTTCTGGATTTTTTTTTCTT | SEQ ID NO: 1605 |
| 207564_x_at | TAAGCTAAAATGTTACTCCCTGTTT | SEQ ID NO: 1606 |
| 207564_x_at | TACTCCCTGTTTTAGTTTCTGAACT | SEQ ID NO: 1607 |
| 207564_x_at | GGGACTTTGCTGGTGTAGTCTTTTT | SEQ ID NO: 1608 |
| 207564_x_at | ACCACTTGAGCCTATATCAGTCGTT | SEQ ID NO: 1609 |
| 207564_x_at | ATCAGTCGTTTTAGTGTCTGACCTA | SEQ ID NO: 1610 |
| 207564_x_at | GTCTGACCTAATATTTGGAGCTATC | SEQ ID NO: 1611 |
| 207564_x_at | GGAGCTATCAGTGCTTTGTTGATTT | SEQ ID NO: 1612 |
| 207564_x_at | AGATTTTTTCTGGTCCATTTCCCAT | SEQ ID NO: 1613 |
| 207564_x_at | TCACCCTTAAAATTCTCCTGTAACT | SEQ ID NO: 1614 |
| 207564_x_at | AAGCCTGATTCAAAACATCCTAGGG | SEQ ID NO: 1615 |
| 208523_x_at | GGAGAGCTATTCCGTGTACGTGTAC | SEQ ID NO: 1616 |
| 208523_x_at | CAAGGTGCTGAAGCAGGTCCACCCC | SEQ ID NO: 1617 |
| 208523_x_at | GCCTGAACCAGCTAAGTCAGCTCCC | SEQ ID NO: 1618 |
| 208523_x_at | CATCTCGTCCAAGGCTATGGGGATT | SEQ ID NO: 1619 |
| 208523_x_at | GATTATGAACTCCTTCGTCAACGAC | SEQ ID NO: 1620 |
| 208523_x_at | TTTTCGAGCGCATTGCAGGCGAGGC | SEQ ID NO: 1621 |
| 208523_x_at | TCCCGCCTGGCGCATTATAACAAGC | SEQ ID NO: 1622 |
| 208523_x_at | TTATAACAAGCGCTCGACCATCACT | SEQ ID NO: 1623 |
| 208523_x_at | CCAGGGAGATCCAAACGGCTGTGCG | SEQ ID NO: 1624 |
| 208523_x_at | AAACACGCGGTGTCGGAGGGCACCA | SEQ ID NO: 1625 |
| 208523_x_at | GAAGGGCTCCAAGAAGGCGGTGACC | SEQ ID NO: 1626 |
| 208527_x_at | AAGCGCAGCCGCAAGGAGAGCTACT | SEQ ID NO: 1627 |
| 208527_x_at | GAGAGCTACTCCGTATACGTGTACA | SEQ ID NO: 1628 |
| 208527_x_at | ATGCCTGAGCCAGCGAAATCCGCTC | SEQ ID NO: 1629 |
| 208527_x_at | GCATCTCCTCTAAAGCCATGGGGAT | SEQ ID NO: 1630 |
| 208527_x_at | TGTCAACGACATCTTCGAGCGCATC | SEQ ID NO: 1631 |
| 208527_x_at | GCATTACAACAAGCGCTCGACCATC | SEQ ID NO: 1632 |
| 208527_x_at | TCGACCATCACCTCCAGGGAGATCC | SEQ ID NO: 1633 |
| 208527_x_at | GGCCAAGCACGCTGTGTCAGAGGGC | SEQ ID NO: 1634 |
| 208527_x_at | GTCAGAGGGCACCAAGGCCGTTACC | SEQ ID NO: 1635 |
| 208527_x_at | TTACCAAGTACACCAGCTCCAAGTA | SEQ ID NO: 1636 |
| 208527_x_at | GAAGGGCTCCAAGAAGGCCGTGACC | SEQ ID NO: 1637 |
| 208707_at | GTTGACCCTGCAGTTCGGTTATGCA | SEQ ID NO: 1638 |
| 208707_at | GAGGATTCACTTGGGTGTTGGGATC | SEQ ID NO: 1639 |
| 208707_at | CAAATTTGGATTCTGTCCCAGGCCT | SEQ ID NO: 1640 |
| 208707_at | TTCTGTCCCAGGCCTTACTGTAAAA | SEQ ID NO: 1641 |
| 208707_at | ACTAGGGGATTGCCTTTCCATATCT | SEQ ID NO: 1642 |
| 208707_at | CATATCTGCTGGGGGTGGAGACCCT | SEQ ID NO: 1643 |
| 208707_at | CACTCAATCCCACTGGAAGCCTAAT | SEQ ID NO: 1644 |
| 208707_at | GAAGCAATGCCTGGCTGGGGCAGTA | SEQ ID NO: 1645 |
| 208707_at | ATTCCACCCAATTTTGCTATGAGCC | SEQ ID NO: 1646 |
| 208707_at | GCTATGAGCCTAAAACCTCTTTAAA | SEQ ID NO: 1647 |
| 208707_at | ACACTGTTTACAAGAGCATCACCTA | SEQ ID NO: 1648 |
| 208820_at | TGCAATATGCTAATCCCACTTTACA | SEQ ID NO: 1649 |
| 208820_at | ACCTGCCTTTTACTTTCGTGTGGAT | SEQ ID NO: 1650 |
| 208820_at | TATGTGAAGCATTGGGTCGGGAACT | SEQ ID NO: 1651 |
| 208820_at | GGGTCGGGAACTAGCTGTAGAACAC | SEQ ID NO: 1652 |
| 208820_at | GAATAATGTGCCAGTTTTTTGTAGC | SEQ ID NO: 1653 |
| 208820_at | AAATGCTTTGTACCAGAGCACCTCC | SEQ ID NO: 1654 |
| 208820_at | CAGAGCACCTCCAAACTGCATTGAG | SEQ ID NO: 1655 |
| 208820_at | AAAGCCATGTTGACTATTTTACAGC | SEQ ID NO: 1656 |
| 208820_at | ACAGCCACTGGAGTTAACTAACCCT | SEQ ID NO: 1657 |
| 208820_at | TTTCTTTTGATGTCCAGTTACACCA | SEQ ID NO: 1658 |
| 208820_at | GTTACACCATCCATTCTGTTAATTT | SEQ ID NO: 1659 |
| 208891_at | AATTGTGCTCTTTTCTAATCCAAAG | SEQ ID NO: 1660 |
| 208891_at | CAAAGGGTATATTTGCAGCATGCTT | SEQ ID NO: 1661 |
| 208891_at | AATAAAAAAACCTTCAGCTGTGCTA | SEQ ID NO: 1662 |
| 208891_at | CTGTGCTAAACAGTATATTACCTCT | SEQ ID NO: 1663 |
| 208891_at | ATATTACCTCTGTATAAAATTCTTC | SEQ ID NO: 1664 |
| 208891_at | AATTCTTCAGGGAGTGTCACCTCAA | SEQ ID NO: 1665 |
| 208891_at | GAGTGTCACCTCAAATGCAATACTT | SEQ ID NO: 1666 |
| 208891_at | TGCAATACTTTGGGTTGGTTTCTTT | SEQ ID NO: 1667 |
| 208891_at | GTGTGTGAGCATGGGTACCCATTTG | SEQ ID NO: 1668 |
| 208891_at | ATGGGTACCCATTTGATAAGAGAAA | SEQ ID NO: 1669 |
| 208891_at | AATTCTCCATTATGTTCGTGGTGTA | SEQ ID NO: 1670 |
| 208988_at | GTTGCTGATTTAGAGTCAATCTCCA | SEQ ID NO: 1671 |
| 208988_at | TAGAGTCAATCTCCAATGTTGTGCT | SEQ ID NO: 1672 |
| 208988_at | GGGATAAGTCTTATGCTATCTCAGT | SEQ ID NO: 1673 |
| 208988_at | TATGCTATCTCAGTTGACACATTGA | SEQ ID NO: 1674 |
| 208988_at | CAGTTGACACATTGAGGTTATTTTG | SEQ ID NO: 1675 |
| 208988_at | GAAGCTAGTTGGACTTTGTTTTGTT | SEQ ID NO: 1676 |
| 208988_at | TGTTTTCCAAAAGTTCTCCACTATT | SEQ ID NO: 1677 |
| 208988_at | AAGTTCTCCACTATTGGTTTTAGAG | SEQ ID NO: 1678 |
| 208988_at | AGCAAGGACATCTTTCCTCTGACAC | SEQ ID NO: 1679 |
| 208988_at | ACGTGGGAATGGGTGATATTTGTGT | SEQ ID NO: 1680 |
| 208988_at | GAAATAGCCTCCAATGGGAAATATT | SEQ ID NO: 1681 |
| 209020_at | GTGAGAAGACATCTCTTTCTGCTCA | SEQ ID NO: 1682 |
| 209020_at | CAGGGGCAGTCGTTGAGCCTTTGAG | SEQ ID NO: 1683 |
| 209020_at | CCCCAAGCAAGTCTCAAAGCCAGTG | SEQ ID NO: 1684 |
| 209020_at | CAGTGATCTCTCTGACTTTCAATCA | SEQ ID NO: 1685 |
| 209020_at | ACCAGGGGCAAGCCATGCACATGCA | SEQ ID NO: 1686 |
| 209020_at | TATTCCTTTTCAGGCCTGCAGAGTG | SEQ ID NO: 1687 |
| 209020_at | GGCTCCAGAACGAAGATCCACACTT | SEQ ID NO: 1688 |
| 209020_at | TTGAGGACTACTCTCAGTCGCTGCA | SEQ ID NO: 1689 |
| 209020_at | ACGCCAGAACTCTGTCTGGCTCTCC | SEQ ID NO: 1690 |
| 209020_at | CCGATCCTGTTCTGAGCAAGCTCGA | SEQ ID NO: 1691 |
| 209020_at | GCAAGCTCGAGTCTTCGTGGATGAT | SEQ ID NO: 1692 |
| 209146_at | GAACCTCATCAATTGATAGCAGTGA | SEQ ID NO: 1693 |
| 209146_at | GTGAGTGACTGAAGCTTCCAAATCA | SEQ ID NO: 1694 |
| 209146_at | ATCAAGAAAAGCCGGCACCAAGAAC | SEQ ID NO: 1695 |
| 209146_at | GGCACCAAGAACTTCCATTCTAATC | SEQ ID NO: 1696 |
| 209146_at | TAATCTAGAGCTGACCAGTTTGAGC | SEQ ID NO: 1697 |
| 209146_at | GATTGCAGTGCAGTACTGGCATTTC | SEQ ID NO: 1698 |
| 209146_at | TTACCCTTCCATTTTTGTATATCAA | SEQ ID NO: 1699 |
| 209146_at | GTATATCAAATTTCCATTGTCATTA | SEQ ID NO: 1700 |
| 209146_at | GTATCTTGAAACTTTGTGAACTGAC | SEQ ID NO: 1701 |
| 209146_at | GTGAACTGACTTGCTGTATTTGCAC | SEQ ID NO: 1702 |
| 209146_at | GTATTTGCACTTTGAGCTCTTGAAA | SEQ ID NO: 1703 |
| 209676_at | TTCTATGCTTATTGTACTTGTTATC | SEQ ID NO: 1704 |
| 209676_at | ACACGTTTGTATCAGAGTTGCTTTT | SEQ ID NO: 1705 |
| 209676_at | GTATCAGAGTTGCTTTTCTAATCTT | SEQ ID NO: 1706 |
| 209676_at | AAATTGCTTATTCTAGGTCTGTAAT | SEQ ID NO: 1707 |
| 209676_at | TAATTTATTAACTGGCTACTGGGAA | SEQ ID NO: 1708 |
| 209676_at | ATTACTTATTTTCTGGATCTATCTG | SEQ ID NO: 1709 |
| 209676_at | AAATTATCATACTACCGGCTACATC | SEQ ID NO: 1710 |
| 209676_at | TACCGGCTACATCAAATCAGTCCTT | SEQ ID NO: 1711 |
| 209676_at | TCAGTCCTTTGATTCCATTTGGTGA | SEQ ID NO: 1712 |
| 209676_at | ATTCAGTCATTGGGAAATGCCGCCC | SEQ ID NO: 1713 |
| 209676_at | AATGCCGCCCATTTAAGTACAGTGG | SEQ ID NO: 1714 |
| 209728_at | CCCCTTGTGCCACACATTGCATTAT | SEQ ID NO: 1715 |
| 209728_at | CCCTTGTGCCACACATTGCATTATT | SEQ ID NO: 1716 |
| 209728_at | CTTGTGCCACACATTGCATTATTAA | SEQ ID NO: 1717 |
| 209728_at | GTGCCACACATTGCATTATTAAATG | SEQ ID NO: 1718 |
| 209728_at | GCATCCAAGCATGATGAGCCCTCTC | SEQ ID NO: 1719 |
| 209728_at | CATCCAAGCATGATGAGCCCTCTCA | SEQ ID NO: 1720 |
| 209728_at | AGCCCTCTCACGGTGCAATGGAGTG | SEQ ID NO: 1721 |
| 209728_at | GCCCTCTCACGGTGCAATGGAGTGC | SEQ ID NO: 1722 |
| 209728_at | CCCTCTCACGGTGCAATGGAGTGCA | SEQ ID NO: 1723 |
| 209728_at | GCAATGGAGTGCACGGTCTGAATCT | SEQ ID NO: 1724 |
| 209728_at | AGCCAACAGGACTCTTGAGCTGAAG | SEQ ID NO: 1725 |
| 209907_s_at | ATCTATGCAAACACCTTTCCCATAA | SEQ ID NO: 1726 |
| 209907_s_at | AACCAAACCCCATAGTACAGTGCCT | SEQ ID NO: 1727 |
| 209907_s_at | TACAGTGCCTTGTCCTAGTGTTCAC | SEQ ID NO: 1728 |
| 209907_s_at | AGTGTTCACATGTTCAGCTCTGTTT | SEQ ID NO: 1729 |
| 209907_s_at | GATGCCAAGGTTTCCATTTTCAGGG | SEQ ID NO: 1730 |
| 209907_s_at | TTACCGCTCGGTTGAATGTGTCCAC | SEQ ID NO: 1731 |
| 209907_s_at | TTGGTGACGCTGTAACCATTCCACG | SEQ ID NO: 1732 |
| 209907_s_at | CACTTGGCGCGGCCTGATACTGAAA | SEQ ID NO: 1733 |
| 209907_s_at | TAGCGTCTACTCGTGCACTGAATAA | SEQ ID NO: 1734 |
| 209907_s_at | AGATTTTATCACTCTCTGCTAAGAC | SEQ ID NO: 1735 |
| 209907_s_at | AAGCTTTATCATTGCCCATATGTAC | SEQ ID NO: 1736 |
| 209911_x_at | CGTCAACGACATCTTCGAGCGCATC | SEQ ID NO: 1737 |
| 209911_x_at | CCCGCCTGGCGCATTACAACAAGCG | SEQ ID NO: 1738 |
| 209911_x_at | GCATTACAACAAGCGCTCGACCATC | SEQ ID NO: 1739 |
| 209911_x_at | TCGACCATCACCTCCAGGGAGATCC | SEQ ID NO: 1740 |
| 209911_x_at | TCACCAAGTACACCAGTTCCAAGTA | SEQ ID NO: 1741 |
| 209911_x_at | GAACTTAGGAAGTCTCATCTGCCTG | SEQ ID NO: 1742 |
| 209911_x_at | TGACTGTGTGGATCCCACCCAAATC | SEQ ID NO: 1743 |
| 209911_x_at | AAATCCAACTCATCCTGGTTTGCTG | SEQ ID NO: 1744 |
| 209911_x_at | AGGTGTTTGCACTTCATGTTACTTT | SEQ ID NO: 1745 |
| 209911_x_at | ATTTACTTCTGTTACAGACCTAGTT | SEQ ID NO: 1746 |
| 209911_x_at | TACTTGCCATGGACTACCTTTGCTA | SEQ ID NO: 1747 |
| 209994_s_at | GAAAAGGTTGTCCAAGAAGCCCTGG | SEQ ID NO: 1748 |
| 209994_s_at | GAAGCCCTGGACAAAGCCAGAGAAG | SEQ ID NO: 1749 |
| 209994_s_at | ACAAAGCCAGAGAAGGCCGCACCTG | SEQ ID NO: 1750 |
| 209994_s_at | CACCTGCATTGTGATTGCTCACCGC | SEQ ID NO: 1751 |
| 209994_s_at | CACCATCCAGAATGCAGACTTAATA | SEQ ID NO: 1752 |
| 209994_s_at | GCAGACTTAATAGTGGTGTTTCAGA | SEQ ID NO: 1753 |
| 209994_s_at | AGAGTCAAGGAGCATGGCACGCATC | SEQ ID NO: 1754 |
| 209994_s_at | GTCAAGGAGCATGGCACGCATCAGC | SEQ ID NO: 1755 |
| 209994_s_at | TCAAGGAGCATGGCACGCATCAGCA | SEQ ID NO: 1756 |
| 209994_s_at | ATCTATTTTTCAATGGTCAGTGTCC | SEQ ID NO: 1757 |
| 209994_s_at | TTTTTCAATGGTCAGTGTCCAGGCT | SEQ ID NO: 1758 |
| 210664_s_at | GCCAGATTTCTGCTTTTTGGAAGAA | SEQ ID NO: 1759 |
| 210664_s_at | AAGATCCTGGAATATGTCGAGGTTA | SEQ ID NO: 1760 |
| 210664_s_at | GTCGAGGTTATATTACCAGGTATTT | SEQ ID NO: 1761 |
| 210664_s_at | GAACGTTTCAAGTATGGTGGATGCC | SEQ ID NO: 1762 |
| 210664_s_at | CAAGTATGGTGGATGCCTGGGCAAT | SEQ ID NO: 1763 |
| 210664_s_at | GATGCCTGGGCAATATGAACAATTT | SEQ ID NO: 1764 |
| 210664_s_at | GAGACACTGGAAGAATGCAAGAACA | SEQ ID NO: 1765 |
| 210664_s_at | GATGGTCCGAATGGTTTCCAGGTGG | SEQ ID NO: 1766 |
| 210664_s_at | AATTATGGAACCCAGCTCAATGCTG | SEQ ID NO: 1767 |
| 210664_s_at | AATGCTGTGAATAACTCCCTGACTC | SEQ ID NO: 1768 |
| 210664_s_at | CCTGACTCCGCAATCAACCAAGGTT | SEQ ID NO: 1769 |
| 210665_at | GATTGGATAGCATTTCATGCCTATG | SEQ ID NO: 1770 |
| 210665_at | CATGCCTATGTTAATATTTGTGCTT | SEQ ID NO: 1771 |
| 210665_at | TTATATGTATACGTGATGCCTTTGT | SEQ ID NO: 1772 |
| 210665_at | GTGATGCCTTTGTAGCATACTGCTA | SEQ ID NO: 1773 |
| 210665_at | AAATGATGGTTGGAAGAATGCGGCT | SEQ ID NO: 1774 |
| 210665_at | GAATGCGGCTCATATTTACCAAGTC | SEQ ID NO: 1775 |
| 210665_at | GCGGCTCATATTTACCAAGTCTTTC | SEQ ID NO: 1776 |
| 210665_at | TTACCAAGTCTTTCTGAACGCCTTC | SEQ ID NO: 1777 |
| 210665_at | GCCTTCTGCATTCATGCATCCATGT | SEQ ID NO: 1778 |
| 210665_at | TCATGCATCCATGTTCTTTCTAGGA | SEQ ID NO: 1779 |
| 210665_at | CATGTTCTTTCTAGGATTGGATAGC | SEQ ID NO: 1780 |
| 210942_s_at | TCAGAAACCTAAACACCCAACAACA | SEQ ID NO: 1781 |
| 210942_s_at | ACAGGAATTATTGCCATCACATTGG | SEQ ID NO: 1782 |
| 210942_s_at | ATGTCACGAAGTTCACCTAGCTGGT | SEQ ID NO: 1783 |
| 210942_s_at | TTAAATACAACTTTTCTGACCTCAA | SEQ ID NO: 1784 |
| 210942_s_at | TGACCTCAAGAGTCCTTTGCACTAC | SEQ ID NO: 1785 |
| 210942_s_at | GCAGAGCAGCTCTTTTTGAAGGACA | SEQ ID NO: 1786 |
| 210942_s_at | AAAACCTCGTAATCAACTTGACTCA | SEQ ID NO: 1787 |
| 210942_s_at | GACTCAAGATTGACTCTACAGACTC | SEQ ID NO: 1788 |
| 210942_s_at | ATATGTTGGATGCACTCGTCAAATA | SEQ ID NO: 1789 |
| 210942_s_at | GATTCATAACCACCAGCTTAATTTC | SEQ ID NO: 1790 |
| 210942_s_at | GAAACCAGCCTTAAACCTGATTTAT | SEQ ID NO: 1791 |
| 212176_at | CAAAGTTGAAAGTGTCCTTTCTCTC | SEQ ID NO: 1792 |
| 212176_at | CTCCCCGTCGTAAACGCTGAGGAAT | SEQ ID NO: 1793 |
| 212176_at | GGCAAGAATGCCATGATGTTCTTTA | SEQ ID NO: 1794 |
| 212176_at | GAGTTTTAAGGGCTTGTCTCATTAT | SEQ ID NO: 1795 |
| 212176_at | GGGCTTGTCTCATTATAGAGGCACA | SEQ ID NO: 1796 |
| 212176_at | GGCACATTGTGGCTGTGTAGGTGAA | SEQ ID NO: 1797 |
| 212176_at | ATAGGTGTACTTTTTCCAATGCTGC | SEQ ID NO: 1798 |
| 212176_at | TCCAATGCTGCTCCAAGTTACTTAA | SEQ ID NO: 1799 |
| 212176_at | ATAAACATGCCATTCTCTTTCAGCT | SEQ ID NO: 1800 |
| 212176_at | TCTTTCAGCTGTAATGTTCTTAAAA | SEQ ID NO: 1801 |
| 212176_at | TTATTCTTGAATGTACTGTGATGTC | SEQ ID NO: 1802 |
| 212179_at | AGTATGCCTTCTTACCAGCAATAGT | SEQ ID NO: 1803 |
| 212179_at | ATCATGCCAGATTTTTGCCAAGATC | SEQ ID NO: 1804 |
| 212179_at | CCAAGATCAGTGTTTCCTCAACATG | SEQ ID NO: 1805 |
| 212179_at | GTATAGTGTGCTCTTGTACCTCTAC | SEQ ID NO: 1806 |
| 212179_at | GTGCTCTTGTACCTCTACATAGATT | SEQ ID NO: 1807 |
| 212179_at | AGCAGTTACACATTTATCTAAAGGA | SEQ ID NO: 1808 |
| 212179_at | AATGCATGTTTACCAAAATGGCTGT | SEQ ID NO: 1809 |
| 212179_at | TTAGACATCGATCACATCTGGAGAC | SEQ ID NO: 1810 |
| 212179_at | GTAGGCGAGCTAACACAGTGTACCT | SEQ ID NO: 1811 |
| 212179_at | ACACAGTGTACCTAATTGCAGAATT | SEQ ID NO: 1812 |
| 212179_at | AGTTGTATAACATTTTCATATCTTA | SEQ ID NO: 1813 |
| 212314_at | TATTTTGGTACCTGTGCTTGCCACA | SEQ ID NO: 1814 |
| 212314_at | TTGATAGATTTCTCTTTGACTTCCA | SEQ ID NO: 1815 |
| 212314_at | TTGACTTCCAAGACCTAGCAGTTAT | SEQ ID NO: 1816 |
| 212314_at | GTCCTAGTGCTTCCGAATCATTTAA | SEQ ID NO: 1817 |
| 212314_at | AATGGCATTGTCGGATATCTTTTAC | SEQ ID NO: 1818 |
| 212314_at | ATCTTTTACATTTCAATTGCAATCC | SEQ ID NO: 1819 |
| 212314_at | AGTACTTAACTGTAGTCTTCTCCAT | SEQ ID NO: 1820 |
| 212314_at | GTAGTCTTCTCCATGAATTACACGT | SEQ ID NO: 1821 |
| 212314_at | GCCTCTAGCTTATAGTTTCATCCCT | SEQ ID NO: 1822 |
| 212314_at | GCCTGCGTGAGTCTGTACAGGGATA | SEQ ID NO: 1823 |
| 212314_at | GGTCCAAACTACTCTTTGCACTACT | SEQ ID NO: 1824 |
| 212764_at | GATGCAATTGGTTCTCCTGCATTGA | SEQ ID NO: 1825 |
| 212764_at | GTTAACATTTATACTTGCCTTGGAC | SEQ ID NO: 1826 |
| 212764_at | TACTTGCCTTGGACTGTAGAACAGA | SEQ ID NO: 1827 |
| 212764_at | TACAATCAAGTCATTTTACCTTTAC | SEQ ID NO: 1828 |
| 212764_at | ATAGCATGATGCTCTGCAGTTTTAT | SEQ ID NO: 1829 |
| 212764_at | TAACCATACAACTCTCATTTCCTTA | SEQ ID NO: 1830 |
| 212764_at | AACTCTCATTTCCTTAGTAAGCCAA | SEQ ID NO: 1831 |
| 212764_at | AATGTTTAACATTTTGTGCCAATTT | SEQ ID NO: 1832 |
| 212764_at | GCCAATTTGTTCCTGTATTCATGTA | SEQ ID NO: 1833 |
| 212764_at | GTTACAGATCTGACTCTTCATTTTT | SEQ ID NO: 1834 |
| 212764_at | AGTTCCTTGTTACATCATGGTCATT | SEQ ID NO: 1835 |
| 212958_x_at | AATTTCCACAGATACTTCCCTTAGA | SEQ ID NO: 1836 |
| 212958_x_at | TGAGCGAGGCCTTGTCAATTTTAAG | SEQ ID NO: 1837 |
| 212958_x_at | TAGGAAGGACCACAACATGACCCGT | SEQ ID NO: 1838 |
| 212958_x_at | TACACACTTTATTTACTTCGTTTTG | SEQ ID NO: 1839 |
| 212958_x_at | GTTGGCTTCTGTTTCTAGTTGAGGA | SEQ ID NO: 1840 |
| 212958_x_at | TCCTCTTTTTCCATCATAATTCTAA | SEQ ID NO: 1841 |
| 212958_x_at | GATTTGCCCATTTACACTTTTGAGA | SEQ ID NO: 1842 |
| 212958_x_at | GTAAATAACCCCATTCTTTGCTTGA | SEQ ID NO: 1843 |
| 212958_x_at | GTATTTTCCCAATAGCACTTTCATT | SEQ ID NO: 1844 |
| 212958_x_at | ATTGCCAGTGTCTTTCTTTGGTGCC | SEQ ID NO: 1845 |
| 212958_x_at | TTCAGCATTCTTAGCCTGTGGCAAT | SEQ ID NO: 1846 |
| 213056_at | AACAACGACAAAAAGCTCCAAGCTG | SEQ ID NO: 1847 |
| 213056_at | AAAGCTCCAAGCTGCAGTGGATTTA | SEQ ID NO: 1848 |
| 213056_at | GGCTAAAACTACCTCATACTTTCCT | SEQ ID NO: 1849 |
| 213056_at | ACTACCTCATACTTTCCTTGGAAGA | SEQ ID NO: 1850 |
| 213056_at | AAAGCAAATGATTTCCATATTCCTG | SEQ ID NO: 1851 |
| 213056_at | ATTTCCATATTCCTGATTGATCTTT | SEQ ID NO: 1852 |
| 213056_at | ACAAGTTTCTTGTTCATATTGTGAA | SEQ ID NO: 1853 |
| 213056_at | GATTTGTTAAACTGGTCCTTAGTCA | SEQ ID NO: 1854 |
| 213056_at | AACTGGTCCTTAGTCATTTGTATAA | SEQ ID NO: 1855 |
| 213056_at | ATTTGTATAGCCTTCTAGAATCAGA | SEQ ID NO: 1856 |
| 213056_at | GAAATAACCTTTTTGCATATTCTTT | SEQ ID NO: 1857 |
| 213355_at | TAAGCTAGTTTTCTGAGGTGTTTTC | SEQ ID NO: 1858 |
| 213355_at | GTGTTTTCACACGTCTTTTTATAGT | SEQ ID NO: 1859 |
| 213355_at | TTATAGTTACTTCATCTTAGATTTT | SEQ ID NO: 1860 |
| 213355_at | AAGGGATATGACTTCCTACTAAGGA | SEQ ID NO: 1861 |
| 213355_at | GTTTACCACAACAATTCTGACTACA | SEQ ID NO: 1862 |
| 213355_at | TTGAGGAGGATATTTGGCTACTGTA | SEQ ID NO: 1863 |
| 213355_at | GGCTACTGTAAACATGGCTGGTGGA | SEQ ID NO: 1864 |
| 213355_at | GGCAAGCCGAAACCACTTGGCTCTG | SEQ ID NO: 1865 |
| 213355_at | GGCTCTGGAAATCTAAGTTCATACT | SEQ ID NO: 1866 |
| 213355_at | TGGTTTAATTAAGCTCTCTCCTGAC | SEQ ID NO: 1867 |
| 213355_at | TGACAACCCCCAGAATTAAATGAAC | SEQ ID NO: 1868 |
| 213541_s_at | CTCGAGGGTTCATGCAGTCAGTGTT | SEQ ID NO: 1869 |
| 213541_s_at | GTCAGTGTTATACCAAACCCAGTGT | SEQ ID NO: 1870 |
| 213541_s_at | AAAAATGCGCATCTCTTTCTTTGTT | SEQ ID NO: 1871 |
| 213541_s_at | TTCAGGACCTCATCATTATGTGGGG | SEQ ID NO: 1872 |
| 213541_s_at | CAGGTAAGAGATGGCCTTCTTGGCT | SEQ ID NO: 1873 |
| 213541_s_at | GGCTGCCACAATCAGAAATCACGCA | SEQ ID NO: 1874 |
| 213541_s_at | GCATTTTGGGTAGGCGGCCTCCAGT | SEQ ID NO: 1875 |
| 213541_s_at | CCAGTTTTCCTTTGAGTCGCGAACG | SEQ ID NO: 1876 |
| 213541_s_at | GTCGCGAACGCTGTGCGTTTGTCAG | SEQ ID NO: 1877 |
| 213541_s_at | ACTACGAGTTGATCTCGGCCAGCCA | SEQ ID NO: 1878 |
| 213541_s_at | TCGGCCAGCCAAAGACACACGACAA | SEQ ID NO: 1879 |
| 213714_at | GTTCTACTCCATACAGTTCACACTG | SEQ ID NO: 1880 |
| 213714_at | GATTGTGACACATTCTTAGTAGCTA | SEQ ID NO: 1881 |
| 213714_at | GCTAGTGTCTGTTCTAGTCACTGCA | SEQ ID NO: 1882 |
| 213714_at | AGTCACTGCACTGGAGTCTACGAGC | SEQ ID NO: 1883 |
| 213714_at | GAGTCTACGAGCCGGAACTCGCTAT | SEQ ID NO: 1884 |
| 213714_at | CGGAACTCGCTATATGCACGTGTGT | SEQ ID NO: 1885 |
| 213714_at | ACGTGTGTGTGTCCGTATGTAAGAA | SEQ ID NO: 1886 |
| 213714_at | GAAAGTGTGCACCGAGTGACTGAAT | SEQ ID NO: 1887 |
| 213714_at | GACTGATATCGAGCATTCTGCCCAC | SEQ ID NO: 1888 |
| 213714_at | GCTTTAACAACCCATTGAGCAGTCA | SEQ ID NO: 1889 |
| 213714_at | GGGAATGTGAGTAAGCTTGCTGCCA | SEQ ID NO: 1890 |
| 213750_at | ACTGTCCTTTTGGGCTTCTATAAAT | SEQ ID NO: 1891 |
| 213750_at | ATATGTAATCGTGCCAGTCTGTTCT | SEQ ID NO: 1892 |
| 213750_at | CAGTCTGTTCTCTGCATGACATAAT | SEQ ID NO: 1893 |
| 213750_at | ATGACATAATTTTCCAGCAATAGCT | SEQ ID NO: 1894 |
| 213750_at | GCTGTGTGGTTTTTGTAATCCTATC | SEQ ID NO: 1895 |
| 213750_at | GTAATCCTATCATCTAGTCAGTTCA | SEQ ID NO: 1896 |
| 213750_at | GTCAGTTCAAGATCTTGCAACACTG | SEQ ID NO: 1897 |
| 213750_at | CAACACTGTGTGATTCTTTGCTCCG | SEQ ID NO: 1898 |
| 213750_at | TTGCTCCGTAGTTCAGTCTTGTTGA | SEQ ID NO: 1899 |
| 213750_at | GACACAGGTGTTTACTTTCCTGTTC | SEQ ID NO: 1900 |
| 213750_at | TTTCCTGTTCTTGCATCTAGTTTCA | SEQ ID NO: 1901 |
| 214327_x_at | GAATCCAGATGGCATGGTTGCTCTA | SEQ ID NO: 1902 |
| 214327_x_at | GGTTGCTCTATTGGACTACCGTGAG | SEQ ID NO: 1903 |
| 214327_x_at | CTACCGTGAGGATGGTGTGACCCCA | SEQ ID NO: 1904 |
| 214327_x_at | GTGACCCCATATATGATTTTCTTTA | SEQ ID NO: 1905 |
| 214327_x_at | ATGTGGCAATTATTTTGGATCTATC | SEQ ID NO: 1906 |
| 214327_x_at | GACTGATGTCATCTTGAGCTCTTCA | SEQ ID NO: 1907 |
| 214327_x_at | TTCCCTTGTACTGTAGTTTGTTTTG | SEQ ID NO: 1908 |
| 214327_x_at | GAGCTCTTCATTTATTTTGACTGTG | SEQ ID NO: 1909 |
| 214327_x_at | TTTGGAGTGGAGGCATTGTTTTTAA | SEQ ID NO: 1910 |
| 214327_x_at | GTTTGTTTTGAATGGCATGTATTTG | SEQ ID NO: 1911 |
| 214327_x_at | TAATTCTAGGTATTTTGTTTGCTTC | SEQ ID NO: 1912 |
| 214349_at | GATACAACGTGTTTCCTAAAAGTAG | SEQ ID NO: 1913 |
| 214349_at | CTTGACTTAACTGCTTCCCTGAAGT | SEQ ID NO: 1914 |
| 214349_at | GACTTAACTGCTTCCCTGAAGTACC | SEQ ID NO: 1915 |
| 214349_at | TAACTGCTTCCCTGAAGTACCGTGA | SEQ ID NO: 1916 |
| 214349_at | GCTTCCCTGAAGTACCGTGAGGTTC | SEQ ID NO: 1917 |
| 214349_at | AGTACCGTGAGGTTCCTGATGTGCG | SEQ ID NO: 1918 |
| 214349_at | CCGTGAGGTTCCTGATGTGCGGGCG | SEQ ID NO: 1919 |
| 214349_at | TTCCTGATGTGCGGGCGGTAGACGG | SEQ ID NO: 1920 |
| 214349_at | ATGTGCGGGCGGTAGACGGTAGGCT | SEQ ID NO: 1921 |
| 214349_at | CGGGCGGTAGACGGTAGGCTTATGC | SEQ ID NO: 1922 |
| 214349_at | TAGACGGTAGGCTTATGCGGCACGC | SEQ ID NO: 1923 |
| 215388_s_at | TATTCATACGTAAAATTTTGGATTA | SEQ ID NO: 1924 |
| 215388_s_at | GAACCACCTCAATGCAAAGATTCTA | SEQ ID NO: 1925 |
| 215388_s_at | GAGGGTAACAAGCGAATAACATGTA | SEQ ID NO: 1926 |
| 215388_s_at | CCACCAAAATGCTTACATCCGTGTG | SEQ ID NO: 1927 |
| 215388_s_at | ACATCCGTGTGTAATATCCCGAGAA | SEQ ID NO: 1928 |
| 215388_s_at | TCCGTGTGTAATATCCCGAGAAATT | SEQ ID NO: 1929 |
| 215388_s_at | AACATAGCATTAAGGTGGACAGCCA | SEQ ID NO: 1930 |
| 215388_s_at | GCATTAAGGTGGACAGCCAAACAGA | SEQ ID NO: 1931 |
| 215388_s_at | GAATTTGTGTGTAAACGGGGATATC | SEQ ID NO: 1932 |
| 215388_s_at | TCACGTTCTCACACATTGCGAACAA | SEQ ID NO: 1933 |
| 215388_s_at | CACACATTGCGAACAACATGTTGGG | SEQ ID NO: 1934 |
| 215779_s_at | AAGCGCAAGCGCAGTCGTAAGGAGA | SEQ ID NO: 1935 |
| 215779_s_at | GCGCAGTCGTAAGGAGAGCTACTCC | SEQ ID NO: 1936 |
| 215779_s_at | GTGCTAAAACAGGTTCACCCCGATA | SEQ ID NO: 1937 |
| 215779_s_at | TAAAACAGGTTCACCCCGATACTGG | SEQ ID NO: 1938 |
| 215779_s_at | AAGCACGCAGTGTCCGAAGGTACCA | SEQ ID NO: 1939 |
| 215779_s_at | GTCCGAAGGTACCAAGGCTGTCACC | SEQ ID NO: 1940 |
| 215779_s_at | GGCTGTCACCAAGTATACAAGCTCC | SEQ ID NO: 1941 |
| 215779_s_at | TACAAGCTCCAAGTAAATGTGTGCT | SEQ ID NO: 1942 |
| 215779_s_at | TCAGCTCCTGCTCCGAAGAAGGGTT | SEQ ID NO: 1943 |
| 215779_s_at | CCTGCTCCGAAGAAGGGTTCCAAGA | SEQ ID NO: 1944 |
| 215779_s_at | GTTCCAAGAAGGCTGTGACCAAGGC | SEQ ID NO: 1945 |
| 217975_at | GTGATGCGTTGGAAGGTTAATCGAA | SEQ ID NO: 1946 |
| 217975_at | CCATCCTTACCCCTATTTAATGTAG | SEQ ID NO: 1947 |
| 217975_at | AACAATACCATATAGCTTGCTTTTT | SEQ ID NO: 1948 |
| 217975_at | CTTTGTCCATATTTCTACTTATAAC | SEQ ID NO: 1949 |
| 217975_at | TATTTCTACTTATAACCTGTTGCTA | SEQ ID NO: 1950 |
| 217975_at | TGTATCTCTTGTTATCTGCATCTCA | SEQ ID NO: 1951 |
| 217975_at | GTTATCTGCATCTCATTGTTTATTG | SEQ ID NO: 1952 |
| 217975_at | GAACCAATCTACAAGTCTCTGTCTT | SEQ ID NO: 1953 |
| 217975_at | AGCCTCTCGGTGGTGGGATTATGAA | SEQ ID NO: 1954 |
| 217975_at | TTATGAATGATTTTTCTCCTTTTGC | SEQ ID NO: 1955 |
| 217975_at | TTCTCCTTTTGCTTGTTAGTATTTT | SEQ ID NO: 1956 |
| 218280_x_at | CCTTCAGTTCCCGGTAGGGCGAGTG | SEQ ID NO: 1957 |
| 218280_x_at | GCATCGCTTGCTGCGCAAAGGCAAC | SEQ ID NO: 1958 |
| 218280_x_at | TCCTCGAGTATCTGACCGCCGAGAT | SEQ ID NO: 1959 |
| 218280_x_at | CGCCGAGATCCTGGAGCTGGCGGGC | SEQ ID NO: 1960 |
| 218280_x_at | CAGGCAGGAGTTTCTCTCGGTGACT | SEQ ID NO: 1961 |
| 218280_x_at | AAGCTGCTGGGCAAAGTCACCATCG | SEQ ID NO: 1962 |
| 218280_x_at | TCTTGCCTAACATCCAGGCCGTACT | SEQ ID NO: 1963 |
| 218280_x_at | AAAGGGCAAGTGAGGCTGACGTCCG | SEQ ID NO: 1964 |
| 218280_x_at | GCGTCTCGAAGGGGCACCTGTGAAC | SEQ ID NO: 1965 |
| 218280_x_at | TACTATCGCTGTCATGTCTGGTCGT | SEQ ID NO: 1966 |
| 218280_x_at | GCAAGCAAGGAGGCAAGGCCCGCGC | SEQ ID NO: 1967 |
| 218332_at | CCCTCCCTTTGGATGCTGGTGAATA | SEQ ID NO: 1968 |
| 218332_at | TTGGATGCTGGTGAATACTGTGTGC | SEQ ID NO: 1969 |
| 218332_at | GATGGGATATGATGCATAGGCTTGG | SEQ ID NO: 1970 |
| 218332_at | TGATGGTTTCCCTAAAGTTATTACG | SEQ ID NO: 1971 |
| 218332_at | GACCCCTGCTTTCGAATTTACATGT | SEQ ID NO: 1972 |
| 218332_at | ATGTTCATGATGTGCCCTTGTTGTA | SEQ ID NO: 1973 |
| 218332_at | ATGATGTGCCCTTGTTGTAAACCTT | SEQ ID NO: 1974 |
| 218332_at | TGTAAACCTTTACCTGTCACTTGTT | SEQ ID NO: 1975 |
| 218332_at | CTGTCACTTGTTTACGTGGGTCTCC | SEQ ID NO: 1976 |
| 218332_at | CACTTGTTTACGTGGGTCTCCTATT | SEQ ID NO: 1977 |
| 218332_at | ATTGTGTTTTTGAACCAGTCTGTAA | SEQ ID NO: 1978 |
| 218627_at | TAATCATTTCTGGGTTCACTGCGAC | SEQ ID NO: 1979 |
| 218627_at | CACTGCGACTCACTGTAGTGCTGGG | SEQ ID NO: 1980 |
| 218627_at | ATCCCCCTTGTAACACTGGAACTGA | SEQ ID NO: 1981 |
| 218627_at | GAGGAGAAATGCCACATACCTTTCC | SEQ ID NO: 1982 |
| 218627_at | ATACCTTTCCCATGGGACCTGTGGT | SEQ ID NO: 1983 |
| 218627_at | CGAGCAGACTTTTGTTCTCGGCGCT | SEQ ID NO: 1984 |
| 218627_at | GGCGCTCCTCACGATGGAGTTTCAT | SEQ ID NO: 1985 |
| 218627_at | GTTTCATGCTTCATTTTCACATCTC | SEQ ID NO: 1986 |
| 218627_at | GAGTACGTGCCTTAATCTTTATCTT | SEQ ID NO: 1987 |
| 218627_at | ATGAACAGAGTGCCTCCTGGTACAC | SEQ ID NO: 1988 |
| 218627_at | AGAATGGGATTTACTCTGCTTTACC | SEQ ID NO: 1989 |
| 218764_at | CACCAAGACGACTGCTTCAGCTTCT | SEQ ID NO: 1990 |
| 218764_at | TCTCTTATCCTTACTTTCTTTAATA | SEQ ID NO: 1991 |
| 218764_at | AAAGGTGCCACAATGCCCAGTATTG | SEQ ID NO: 1992 |
| 218764_at | AGCTTTCATTCATTCTGGAGTCTAC | SEQ ID NO: 1993 |
| 218764_at | ATTCTGTGAAATGCCTCTCCACGTT | SEQ ID NO: 1994 |
| 218764_at | TCTCCACGTTGCATATGTCACACTT | SEQ ID NO: 1995 |
| 218764_at | GTCTGCACATAACTCTTTTTTCACA | SEQ ID NO: 1996 |
| 218764_at | GCCACAACAGCACAGTCAGCGGGTG | SEQ ID NO: 1997 |
| 218764_at | GTCAGCGGGTGAATTACAGGTGCCT | SEQ ID NO: 1998 |
| 218764_at | GTAATCTGATCTTGTCTGTATCGCC | SEQ ID NO: 1999 |
| 218764_at | AGAATTGCAGGCCACTCATGTCAGT | SEQ ID NO: 2000 |
| 218772_x_at | TTTCCATAGCAGGTATTTTCTACTA | SEQ ID NO: 2001 |
| 218772_x_at | TCTGAAGTCTTTTTCATGCCCTTGT | SEQ ID NO: 2002 |
| 218772_x_at | AGCTTGACTTATTTTTTTCTCTCTC | SEQ ID NO: 2003 |
| 218772_x_at | GGAGAAATTTTCTCAGCATTTTGCA | SEQ ID NO: 2004 |
| 218772_x_at | GCATTTTGCATGTTCTTTCTAATCT | SEQ ID NO: 2005 |
| 218772_x_at | GTTCTTTCTAATCTTTGTTGGTCTG | SEQ ID NO: 2006 |
| 218772_x_at | TCAAAAATTTTTCCACTATGTCTTT | SEQ ID NO: 2007 |
| 218772_x_at | TATGTCTTTTTTCTAGTGGCTACTG | SEQ ID NO: 2008 |
| 218772_x_at | GTGGCTACTGTTTTAGTTTTCTAGT | SEQ ID NO: 2009 |
| 218772_x_at | ATCTCTGACAAGCTTTCGTATGGTT | SEQ ID NO: 2010 |
| 218772_x_at | GGTTTTGTTATATCTTCATCTACAT | SEQ ID NO: 2011 |
| 218901_at | TATATTCATCTTTTCAGGGTAAATT | SEQ ID NO: 2012 |
| 218901_at | GAGTTTCTCGTAATGCTCATTTTTA | SEQ ID NO: 2013 |
| 218901_at | CTCATTTTTACATGCTGCTACTAGC | SEQ ID NO: 2014 |
| 218901_at | GTGCCATTGCAATCGTAAGTAGACT | SEQ ID NO: 2015 |
| 218901_at | GTAGACTATGTATTTCCTATAATGA | SEQ ID NO: 2016 |
| 218901_at | TTTAACTTGCCTAGATCCCTGTATT | SEQ ID NO: 2017 |
| 218901_at | TAGATCCCTGTATTCCAAAACCTGC | SEQ ID NO: 2018 |
| 218901_at | TGTATTCCAAAACCTGCTGCATCAT | SEQ ID NO: 2019 |
| 218901_at | CATGATTTCTATGTTTCTTAATGAT | SEQ ID NO: 2020 |
| 218901_at | GGAATTTGTGCGTTCATGCTTTTTC | SEQ ID NO: 2021 |
| 218901_at | GTTCATGCTTTTTCGTATTCTTTAT | SEQ ID NO: 2022 |
| 218971_s_at | CATGCTGACATGTTCTGCCACAGGC | SEQ ID NO: 2023 |
| 218971_s_at | GCGTCATCTACAAGCTGGGTGGCGA | SEQ ID NO: 2024 |
| 218971_s_at | ACTGGAGCACTGCCATGGACTGTGG | SEQ ID NO: 2025 |
| 218971_s_at | AAGGGCCACCCGAGGAAGCAGTATT | SEQ ID NO: 2026 |
| 218971_s_at | AGGACAGGAAAACCACGTGCTCCAC | SEQ ID NO: 2027 |
| 218971_s_at | TGGCAAGGAGGCTCAGGTGCTTCCA | SEQ ID NO: 2028 |
| 218971_s_at | GTGCTTCCATCTGTGGTGACTGGAA | SEQ ID NO: 2029 |
| 218971_s_at | GGAATGGGACCCACGTGGAGTAGGT | SEQ ID NO: 2030 |
| 218971_s_at | GAGTAGGTGACATATGCTTCCCAGA | SEQ ID NO: 2031 |
| 218971_s_at | GTGGCTGTGCCAGGAGTACATGTGA | SEQ ID NO: 2032 |
| 218971_s_at | ATATATGTGCCCATTTATCTTTTTC | SEQ ID NO: 2033 |
| 219054_at | GACAACAATGAAGTAGCCCCTGAAC | SEQ ID NO: 2034 |
| 219054_at | GTAGCCCCTGAACAGCATGGAGTTG | SEQ ID NO: 2035 |
| 219054_at | GAGTTGCTGTGAGTTTGTTCGTTGC | SEQ ID NO: 2036 |
| 219054_at | GTTCGTTGCAGACCTTTGTGTTGGG | SEQ ID NO: 2037 |
| 219054_at | GGTCCTGGGAATCTGAGCTTTGTTC | SEQ ID NO: 2038 |
| 219054_at | CTTTGTTCCCTGTGCATGGTGGATA | SEQ ID NO: 2039 |
| 219054_at | GGGATAGACCTTGTGACAGACCAAT | SEQ ID NO: 2040 |
| 219054_at | GACAGACCAATTCTGTGACCCCTGT | SEQ ID NO: 2041 |
| 219054_at | TGACCCCTGTCTTCTGGGTCACATT | SEQ ID NO: 2042 |
| 219054_at | AAATGTGTATGTGTCCTTGTAAATG | SEQ ID NO: 2043 |
| 219054_at | GCAAGAATGCCACGTACTCAGAGTA | SEQ ID NO: 2044 |
| 219559_at | TGTCCTGCACACTGTAGGATGCTTA | SEQ ID NO: 2045 |
| 219559_at | GATGCTTAAAGGTATCCCTGGCCTC | SEQ ID NO: 2046 |
| 219559_at | CCCAGTCAGACATGACCTCAGAGTC | SEQ ID NO: 2047 |
| 219559_at | CTCAGAGTCTCTGTGTCTCCTAGAA | SEQ ID NO: 2048 |
| 219559_at | CTCCTAGAAGCCTGACAGAGACCCC | SEQ ID NO: 2049 |
| 219559_at | TGGGTGGGTGGCGGGCTAGAGACCC | SEQ ID NO: 2050 |
| 219559_at | CCCTCCGCACTAACAGTGTTCTCAG | SEQ ID NO: 2051 |
| 219559_at | GCCTGGTGATTCTGCTCTCCAGGGA | SEQ ID NO: 2052 |
| 219559_at | CTCCCTTTTCGTTGCCTGAGGAGCT | SEQ ID NO: 2053 |
| 219559_at | GGAGCTGGTGGTTTCATGAGTTAAT | SEQ ID NO: 2054 |
| 219559_at | GTGGAAAAGCACGCCAAAGCCTTAT | SEQ ID NO: 2055 |
| 219648_at | ATGCTGTGAATGCAGCTTGCTTCTC | SEQ ID NO: 2056 |
| 219648_at | GTCCAGCTTCAAAAGTTACTTGCCA | SEQ ID NO: 2057 |
| 219648_at | AGATTTTGCACTTCTGAATTCAGGT | SEQ ID NO: 2058 |
| 219648_at | TCTGCTTAGAGGACTGTGACTTGAA | SEQ ID NO: 2059 |
| 219648_at | TGAGCTTTTTGGTAGCGTCCACAAT | SEQ ID NO: 2060 |
| 219648_at | ATAGGCGAGATCCGTGTTCTCCATT | SEQ ID NO: 2061 |
| 219648_at | TGTAGACCAATTTAACTGCTGTGTT | SEQ ID NO: 2062 |
| 219648_at | TTAGTGCTTAATCTTTGCCTCATGT | SEQ ID NO: 2063 |
| 219648_at | TTCTCTCAATTCTGTAGACTCTCGC | SEQ ID NO: 2064 |
| 219648_at | GCACTGAAGATCTTTGCTGGACCTT | SEQ ID NO: 2065 |
| 219648_at | CTGGACCTTCTTCTCTTCAGAAGAT | SEQ ID NO: 2066 |
| 220122_at | CACCTGTGCTCTGATTAAATCTACA | SEQ ID NO: 2067 |
| 220122_at | AGTAATCCATTACACTTTTCTATGT | SEQ ID NO: 2068 |
| 220122_at | ATTCTGGCTTTAGATCCCGACATTC | SEQ ID NO: 2069 |
| 220122_at | CCGACATTCACTCCTGTGCAAATTA | SEQ ID NO: 2070 |
| 220122_at | GTACATTCACTCCCTCAAGAGAATC | SEQ ID NO: 2071 |
| 220122_at | ATTTCAATCAATCATTCCATCTAAA | SEQ ID NO: 2072 |
| 220122_at | AAATCTCTACAGGACTACATAACAT | SEQ ID NO: 2073 |
| 220122_at | AAACGATTGCCTATCTGAATTTTTA | SEQ ID NO: 2074 |
| 220122_at | TGAATTTTTATACCTACCACTACTT | SEQ ID NO: 2075 |
| 220122_at | GGAAACTATATCCATATCGCTTTTG | SEQ ID NO: 2076 |
| 220122_at | ATCGCTTTTGGTGTCAGATTGTATC | SEQ ID NO: 2077 |
| 221458_at | GGCATGGCTTGGGTATCTCAATTCC | SEQ ID NO: 2078 |
| 221458_at | TCTCAATTCCCTTATAAATCCACTG | SEQ ID NO: 2079 |
| 221458_at | TGCATCATCAAGCACGACCACATTG | SEQ ID NO: 2080 |
| 221458_at | ACATTGTTTCCACCATTTACTCAAC | SEQ ID NO: 2081 |
| 221458_at | ATCCCACTGGCATTGATTTTGATCC | SEQ ID NO: 2082 |
| 221458_at | GGAGGTGAATGGCCAAGTCCTTTTG | SEQ ID NO: 2083 |
| 221458_at | ATCAGTTTCCACATCCTATGTACTA | SEQ ID NO: 2084 |
| 221458_at | AAAGTCTTTATCTGACCCATCAACA | SEQ ID NO: 2085 |
| 221458_at | AGAGAACGGAAAGCAGCCACTACCC | SEQ ID NO: 2086 |
| 221458_at | GCAGCCACTACCCTGGGATTAATCT | SEQ ID NO: 2087 |
| 221458_at | TAATATGTTGGCTTCCTTTTTTTGT | SEQ ID NO: 2088 |
| 221773_at | GAACACATCCAAAATGCATGATTCT | SEQ ID NO: 2089 |
| 221773_at | TATAGATCTGATTCTTTCTTTTCCT | SEQ ID NO: 2090 |
| 221773_at | AACTGGGATTAATGTATGCTCTAGA | SEQ ID NO: 2091 |
| 221773_at | TGTATGCTCTAGATCCATTTATTAG | SEQ ID NO: 2092 |
| 221773_at | ATAACTCACTCATATAGCTCTGCCT | SEQ ID NO: 2093 |
| 221773_at | ATGTCTGCTTAATCAGTGTTAAACT | SEQ ID NO: 2094 |
| 221773_at | ATAACCTGAATGTTGGTCTCTTTGT | SEQ ID NO: 2095 |
| 221773_at | TGGTCTCTTTGTACACATCTTTTCT | SEQ ID NO: 2096 |
| 221773_at | CACATCTTTTCTATGACTGCAAATC | SEQ ID NO: 2097 |
| 221773_at | GACTGCAAATCTTCACTTTATGTAT | SEQ ID NO: 2098 |
| 221773_at | CTTTATGTATCATTTTTACTGTCAT | SEQ ID NO: 2099 |
| 221833_at | AAGCACCAGGGCACGGACAGGAATA | SEQ ID NO: 2100 |
| 221833_at | GTACTGAATTAGCCACTTTCTCCAT | SEQ ID NO: 2101 |
| 221833_at | CTTTCTCCATAGCCAAGTTGCGAAT | SEQ ID NO: 2102 |
| 221833_at | GGCCCCGGCAAGTTGGACAACATGT | SEQ ID NO: 2103 |
| 221833_at | GAGCTTTGGGCGACAGTTGCTACAA | SEQ ID NO: 2104 |
| 221833_at | AACAAGATGGCCACTCTGACATTGA | SEQ ID NO: 2105 |
| 221833_at | GACTGGACACTCAAAAAGACTCGCC | SEQ ID NO: 2106 |
| 221833_at | ATTGTTGGATGCAGTTGTGCCAGTC | SEQ ID NO: 2107 |
| 221833_at | TGGACACTTCGAGGTACCGGTAGGT | SEQ ID NO: 2108 |
| 221833_at | AGCAGTCTGACGGCTCATTTCTGAA | SEQ ID NO: 2109 |
| 221833_at | GTTTACATGCCATAAGTCCTTTTAA | SEQ ID NO: 2110 |
| 221942_s_at | TTTTGCTGGCGTCGTTGGAGTTAAA | SEQ ID NO: 2111 |
| 221942_s_at | TGGAGTTAAAATGCCCCGTTACTGT | SEQ ID NO: 2112 |
| 221942_s_at | AAACAATGTCACTCTGGCTAACAAA | SEQ ID NO: 2113 |
| 221942_s_at | ACTCAAAGACTGTCCTGGTTTCGTG | SEQ ID NO: 2114 |
| 221942_s_at | TCGTGTTTACCCCTCGATCAAGGGA | SEQ ID NO: 2115 |
| 221942_s_at | TTCCACCAAACTTCCCTAGTGAAAT | SEQ ID NO: 2116 |
| 221942_s_at | AGTGAAATCCCCGGAATCTGCCATT | SEQ ID NO: 2117 |
| 221942_s_at | AACAAACTCAAAACCATGCTTCCAA | SEQ ID NO: 2118 |
| 221942_s_at | GTCACAATCTTTCTCCTGTTTAACA | SEQ ID NO: 2119 |
| 221942_s_at | CTGATGAAGTTATGTCTCCCCATGG | SEQ ID NO: 2120 |
| 221942_s_at | CTCCCCATGGAGAACCTATCAAGAT | SEQ ID NO: 2121 |
| 222067_x_at | CCGGCATCTCTTCCAAGGCAATGGG | SEQ ID NO: 2122 |
| 222067_x_at | GGATCATGAATTCCTTCGTCAACGA | SEQ ID NO: 2123 |
| 222067_x_at | CGTCAACGACATCTTCGAGCGCATC | SEQ ID NO: 2124 |
| 222067_x_at | ATTAACGCTACGATGCCTGAACCTA | SEQ ID NO: 2125 |
| 222067_x_at | GCATTACAACAAGCGCTCGACCATC | SEQ ID NO: 2126 |
| 222067_x_at | TCGACCATCACCTCCAGGGAGATCC | SEQ ID NO: 2127 |
| 222067_x_at | GTAAGCATCTTTACACCTAATCCCA | SEQ ID NO: 2128 |
| 222067_x_at | TACACCTAATCCCAAAGGCTCTTTT | SEQ ID NO: 2129 |
| 222067_x_at | TAAGAGCCACGCATGTTTTCAATAA | SEQ ID NO: 2130 |
| 222067_x_at | GCTCCTGCCCCAAAGAAGGGCTCCA | SEQ ID NO: 2131 |
| 222067_x_at | AGGGCTCCAAGAAGGCGGTGACTAA | SEQ ID NO: 2132 |
| 222315_at | GGCCTGCAGTGGATAGAGCCTAGCA | SEQ ID NO: 2133 |
| 222315_at | ATCTCTGACAGTGATTTCCAGCGAC | SEQ ID NO: 2134 |
| 222315_at | CCAGCGACTTTGTCAACACGGTCCG | SEQ ID NO: 2135 |
| 222315_at | TCCGCCCCCAGCAAGTATAAGAGGA | SEQ ID NO: 2136 |
| 222315_at | ACAAATGTCTTTACTGCCTTGTCTT | SEQ ID NO: 2137 |
| 222315_at | CCCTTGCCACTTGTCATTATTCAAG | SEQ ID NO: 2138 |
| 222315_at | TTACCAGCTGTGCTTGCGTTGCAAG | SEQ ID NO: 2139 |
| 222315_at | CTTGCGTTGCAAGACCTGTCACAGT | SEQ ID NO: 2140 |
| 222315_at | CTGCACCATTCAAACTAGCCAACCC | SEQ ID NO: 2141 |
| 222315_at | TCTTCGGGGCTCATGCTAGGCCCGA | SEQ ID NO: 2142 |
| 222315_at | GCTAGGCCCGAGTGCATTCAATAAA | SEQ ID NO: 2143 |
| 222735_at | GAACTTCATATGGCAGTCCATTTAG | SEQ ID NO: 2144 |
| 222735_at | TAAAATCTGGTTCCTTCTTAGCAAA | SEQ ID NO: 2145 |
| 222735_at | AAAACTCTGTGACATAGTTTCTTTT | SEQ ID NO: 2146 |
| 222735_at | TACTCCCCGTATCAGGTATTTTCGA | SEQ ID NO: 2147 |
| 222735_at | AAGTACTCAAGTCACATCACATTCA | SEQ ID NO: 2148 |
| 222735_at | AAACACCAGCAGATACTATTACTTG | SEQ ID NO: 2149 |
| 222735_at | ATTGGGAGGGGGCACTTTTCATAGT | SEQ ID NO: 2150 |
| 222735_at | GGCACTTTTCATAGTCTTGGAATGC | SEQ ID NO: 2151 |
| 222735_at | TATTATATTTGATACTCTTACAGTT | SEQ ID NO: 2152 |
| 222735_at | AATTATTGACCAGTTTTGAAGTTTG | SEQ ID NO: 2153 |
| 222735_at | GAAGGACTCTTGTTTTACACTTGTA | SEQ ID NO: 2154 |
| 222815_at | TGATCTTTAAATTTTCCCACACCAT | SEQ ID NO: 2155 |
| 222815_at | AAATTTTCCCACACCATAAGAGAGG | SEQ ID NO: 2156 |
| 222815_at | AAAGCTATATCATTCCCAGTTATTA | SEQ ID NO: 2157 |
| 222815_at | GTTAACACAAATTCAGCCACATTCT | SEQ ID NO: 2158 |
| 222815_at | GAGTATTGTTTGTTCACCTTTCAGA | SEQ ID NO: 2159 |
| 222815_at | TGTTTGTTCACCTTTCAGACTTGGT | SEQ ID NO: 2160 |
| 222815_at | GACTTGGTGATACTGGACATGTCAG | SEQ ID NO: 2161 |
| 222815_at | AGGATCTTCTAAGTGTATAACTGTC | SEQ ID NO: 2162 |
| 222815_at | GCCCATCACTGTGGCACACTGTAGA | SEQ ID NO: 2163 |
| 222815_at | AAAGCCTATGCTTGTGTAAGTGAAA | SEQ ID NO: 2164 |
| 222815_at | TAGAGGCTCAGTACTTTTCCAATGC | SEQ ID NO: 2165 |
| 225629_s_at | GCTCTATACGTAGTGAGGACCCAGA | SEQ ID NO: 2166 |
| 225629_s_at | GTGAGGACCCAGATTTAGAGAAACT | SEQ ID NO: 2167 |
| 225629_s_at | ATTTATCTCCGCATTTGTGTGTGTG | SEQ ID NO: 2168 |
| 225629_s_at | AACTCTGTAGGCCAATAAACCAACA | SEQ ID NO: 2169 |
| 225629_s_at | AAATAGCTTCCAGAATGTGGTGGTT | SEQ ID NO: 2170 |
| 225629_s_at | GAATGTGGTGGTTCTGGGCAACAAA | SEQ ID NO: 2171 |
| 225629_s_at | GAGATTGTGGCGACGTGGAGATTAA | SEQ ID NO: 2172 |
| 225629_s_at | TGATCAAGTCTTGTCAGTTCGTGCC | SEQ ID NO: 2173 |
| 225629_s_at | TCTTTCCCCATGTTCCCTGGGAAGA | SEQ ID NO: 2174 |
| 225629_s_at | GTTCTGTGCCGCAGCACGCAAAATT | SEQ ID NO: 2175 |
| 225629_s_at | GAATTCTACAGACTAGCTCTATACG | SEQ ID NO: 2176 |
| 226545_at | CATGTGTCTCTGTAATAGGGATAAT | SEQ ID NO: 2177 |
| 226545_at | TCTATCTTATGTTGTCTTGAGGCCA | SEQ ID NO: 2178 |
| 226545_at | GAGGCCAAGATTTACCACGTTTGCC | SEQ ID NO: 2179 |
| 226545_at | TTACCACGTTTGCCCAGTGTATTGA | SEQ ID NO: 2180 |
| 226545_at | GGTAGAAGGTAGTTCCATGTTCCAT | SEQ ID NO: 2181 |
| 226545_at | TCCATGTTCCATTTGTAGATCTTTA | SEQ ID NO: 2182 |
| 226545_at | AGAATGTGGCTCAGTTCTGGTCCTT | SEQ ID NO: 2183 |
| 226545_at | GGTCCTTCAAGCCTGTATGGTTTGG | SEQ ID NO: 2184 |
| 226545_at | TTGGATTTTCAGTAGGGGACAGTTG | SEQ ID NO: 2185 |
| 226545_at | GGAGTCAATCTCTTTGGTACACAGG | SEQ ID NO: 2186 |
| 226545_at | TTCATTCACGAATCTCTTATTTTGG | SEQ ID NO: 2187 |
| 226547_at | AATATTGGTACCTGTCATTTTTTCA | SEQ ID NO: 2188 |
| 226547_at | TGTTAGTGACTTTGATGCCTTTTAA | SEQ ID NO: 2189 |
| 226547_at | AAAGAGATCTCTAGCGTGTGTGAAT | SEQ ID NO: 2190 |
| 226547_at | GCGTGTGTGAATAGAGCTCCAGATG | SEQ ID NO: 2191 |
| 226547_at | GCTCCAGATGCCTCTAAAAGCCGCA | SEQ ID NO: 2192 |
| 226547_at | AGCCGCATGTACAAAGGAAGCCACG | SEQ ID NO: 2193 |
| 226547_at | AAAGGAAGCCACGTCTATCCTGTCT | SEQ ID NO: 2194 |
| 226547_at | TGCTTTTCCTGTTTTGTAACCTCTT | SEQ ID NO: 2195 |
| 226547_at | TTGTAACCTCTTTGTACTTTGTTCA | SEQ ID NO: 2196 |
| 226547_at | GTACTTTGTTCATGGTGACTTGTAA | SEQ ID NO: 2197 |
| 226547_at | GGAAGGGGTGCCTAGATGCCTTTGT | SEQ ID NO: 2198 |
| 226985_at | GGCCTCTGAAGAGTCAAGGTCTGCT | SEQ ID NO: 2199 |
| 226985_at | TGTGTTTACCTCACTCAAGCTGACA | SEQ ID NO: 2200 |
| 226985_at | GGGAATCTATCCTTCTTTTAGACAC | SEQ ID NO: 2201 |
| 226985_at | GACACACGGTAATCCTTGGGCTGTA | SEQ ID NO: 2202 |
| 226985_at | GGGCTGTATTACTGAAGGCTTTTTA | SEQ ID NO: 2203 |
| 226985_at | AGGTGAATTCCTGGTCTTGGCAGAT | SEQ ID NO: 2204 |
| 226985_at | GGAGCACAGAAGTCGTGGCCTGAGG | SEQ ID NO: 2205 |
| 226985_at | GGCCTGAGGCTGTTCTATGGGCACT | SEQ ID NO: 2206 |
| 226985_at | TGGGCACTTGGGGCTAAATCGCCTC | SEQ ID NO: 2207 |
| 226985_at | AATCGCCTCCTGAGGGTGACTGTTG | SEQ ID NO: 2208 |
| 226985_at | GTGACTGTTGCTTATTCTGCTGGAC | SEQ ID NO: 2209 |
| 228465_at | GAAGTGGTAGGCAAGAGTCTCTGTG | SEQ ID NO: 2210 |
| 228465_at | GAGTCTCTGTGTTACCATGGGAACG | SEQ ID NO: 2211 |
| 228465_at | GAACGATTAAGTTTTCCAAGGTGCA | SEQ ID NO: 2212 |
| 228465_at | CCTCATTCCAGCTTCAGGGTCAATG | SEQ ID NO: 2213 |
| 228465_at | GGGTCAATGACTTACTAGCTCAGAG | SEQ ID NO: 2214 |
| 228465_at | ACATACCTACTATCTGTACAGAGTG | SEQ ID NO: 2215 |
| 228465_at | GAGTGACTCTCATTACCCAGAGAAC | SEQ ID NO: 2216 |
| 228465_at | GGGGAGTACTTAAGGTGTATGAGCA | SEQ ID NO: 2217 |
| 228465_at | AACAAATTGTTATCCAGGTCACTCC | SEQ ID NO: 2218 |
| 228465_at | TCACTCCAGAACTGTTGTATACAGA | SEQ ID NO: 2219 |
| 228465_at | TTGTGCCCTGAAAATTGTATCAACA | SEQ ID NO: 2220 |
| 228570_at | TAAACCTATTTCCTAGCATGCCTTC | SEQ ID NO: 2221 |
| 228570_at | GTTGTGCCAGACCCTAGATTGTGAA | SEQ ID NO: 2222 |
| 228570_at | CACTGTTCTTCTGTTGTACGAGCTC | SEQ ID NO: 2223 |
| 228570_at | CAATGTCACATCGCTTCATGGGCAT | SEQ ID NO: 2224 |
| 228570_at | GGCATGGCCCATGGAGCATCTGGGT | SEQ ID NO: 2225 |
| 228570_at | TATTGGCTCTTCTGCGAGGCTGATA | SEQ ID NO: 2226 |
| 228570_at | CCTCTCTTCCACATGATCATTTGCA | SEQ ID NO: 2227 |
| 228570_at | CTGCGTGGATGTTTCCTTAACCTCA | SEQ ID NO: 2228 |
| 228570_at | TGTCTAATGCTAGTTCAGGGCCTCC | SEQ ID NO: 2229 |
| 228570_at | GGCCTCCAGGCATTGATTTGTACAG | SEQ ID NO: 2230 |
| 228570_at | GGTAACTCCCAATGAGGCTTCTGTT | SEQ ID NO: 2231 |
| 228857_at | GGTGGGCGTGGTACTGAGAGTCCCA | SEQ ID NO: 2232 |
| 228857_at | GTGAGGGGAGTGCCCTCAGGCAGGC | SEQ ID NO: 2233 |
| 228857_at | GGAGGGAACAGCGCTGACATTCAGC | SEQ ID NO: 2234 |
| 228857_at | TCAGCTGGTTCGCACTGATACGGCT | SEQ ID NO: 2235 |
| 228857_at | GATACGGCTCAACCAGTTTGTTAAA | SEQ ID NO: 2236 |
| 228857_at | GGACTTCCCGCTGCATTTGAGAAGC | SEQ ID NO: 2237 |
| 228857_at | TTGAGAAGCTTTGCAGCGCCATCTG | SEQ ID NO: 2238 |
| 228857_at | TGCTTTGCGCCTTCATCTTGAAGCA | SEQ ID NO: 2239 |
| 228857_at | GAAGCACTCTGAAATTGCCTGTTTA | SEQ ID NO: 2240 |
| 228857_at | GAATCATGGAGTTGCTACTGCTTCT | SEQ ID NO: 2241 |
| 228857_at | AGTGCATTGTCGTTCTTGTGTCAGT | SEQ ID NO: 2242 |
| 228904_at | AACTGTGAGAGATGTCTGGGCCTGC | SEQ ID NO: 2243 |
| 228904_at | GAGATGTCTGGGCCTGCAGAAGTCC | SEQ ID NO: 2244 |
| 228904_at | GCAGAAGTCCAGCATTGCTCAAAAA | SEQ ID NO: 2245 |
| 228904_at | ATTATTTATCCCCCTACATTATGTA | SEQ ID NO: 2246 |
| 228904_at | AGGACATTGTGTTTCCTGTCATGTA | SEQ ID NO: 2247 |
| 228904_at | AAAGGCATGAACTCAGCTCCTAATC | SEQ ID NO: 2248 |
| 228904_at | ACTCAGCTCCTAATCGTCACTGTAT | SEQ ID NO: 2249 |
| 228904_at | AATCGTCACTGTATAGTCCTGAATT | SEQ ID NO: 2250 |
| 228904_at | TAGAGTTAATTCCCTCTTGGAACTT | SEQ ID NO: 2251 |
| 228904_at | TTTCTTTGTTCTTCAGTAGTTACTT | SEQ ID NO: 2252 |
| 228904_at | AAGGGTTGTCTGTCAAACAATTCTT | SEQ ID NO: 2253 |
| 228915_at | GAAAAAAGCTATCAGCTGTATGTTA | SEQ ID NO: 2254 |
| 228915_at | AGAGAGACTCTTACTAACATGTTGT | SEQ ID NO: 2255 |
| 228915_at | ATTTTATGGTTTCCATGCTTTTGTA | SEQ ID NO: 2256 |
| 228915_at | TCCATGCTTTTGTAATCCTAAAAAT | SEQ ID NO: 2257 |
| 228915_at | AAAATATTAATGTCTAGTTGTTCTA | SEQ ID NO: 2258 |
| 228915_at | TTATAACCACATTTGCGCTCTATGC | SEQ ID NO: 2259 |
| 228915_at | CACATTTGCGCTCTATGCAAGCCCT | SEQ ID NO: 2260 |
| 228915_at | CGCTCTATGCAAGCCCTTGGAACAG | SEQ ID NO: 2261 |
| 228915_at | AATTTTTCTATGGTAGCCTAGTTAT | SEQ ID NO: 2262 |
| 228915_at | GTAGCCTAGTTATTTGAGCCTGGTT | SEQ ID NO: 2263 |
| 228915_at | ATTTGAGCCTGGTTTCAATGTGAGA | SEQ ID NO: 2264 |
| 229287_at | GATTAAACCTATACAAGTCTGGCAA | SEQ ID NO: 2265 |
| 229287_at | TACAAGTCTGGCAATGAGCTCTGCA | SEQ ID NO: 2266 |
| 229287_at | AATGAGCTCTGCATGAGGAAATGGA | SEQ ID NO: 2267 |
| 229287_at | TCCTTTTCTGATCATGGGCTCTGGA | SEQ ID NO: 2268 |
| 229287_at | GATCATGGGCTCTGGAAAGTATTCA | SEQ ID NO: 2269 |
| 229287_at | GAAAGTATTCATGGCCTTTACCAGC | SEQ ID NO: 2270 |
| 229287_at | ACCAGCATTCAGTATAAACCAGAGA | SEQ ID NO: 2271 |
| 229287_at | ATATGTACTTACGTGTGTCTGTGAG | SEQ ID NO: 2272 |
| 229287_at | TGTGTGTCTGAGTGTTATTCTGAAC | SEQ ID NO: 2273 |
| 229287_at | GAGTGTTATTCTGAACAGCTTGTAA | SEQ ID NO: 2274 |
| 229287_at | AAGCTGAGTTCTTTTGGCAAATATA | SEQ ID NO: 2275 |
| 230389_at | ATATGTTTAGAGATGCCGCCAGAAC | SEQ ID NO: 2276 |
| 230389_at | AGCATGTTCTCCATTTGCAGTCTAC | SEQ ID NO: 2277 |
| 230389_at | GAAAATCCTTACCAGTTGTTTGTCA | SEQ ID NO: 2278 |
| 230389_at | TCTTGTTCTCTTGCTGGTTATTGGC | SEQ ID NO: 2279 |
| 230389_at | GCTGGTTATTGGCAGACTCAGTCTT | SEQ ID NO: 2280 |
| 230389_at | GATAGGGAAACCCACGTATGCCTTT | SEQ ID NO: 2281 |
| 230389_at | ATGCCTTTGAGGCTAGGGACTATGT | SEQ ID NO: 2282 |
| 230389_at | GGGACTATGTTGTAAGTTCACCTGT | SEQ ID NO: 2283 |
| 230389_at | GTTCACCTGTGATGGCCAGGTCATA | SEQ ID NO: 2284 |
| 230389_at | AGACTGGGGACCCAGAGGCACTTGT | SEQ ID NO: 2285 |
| 230389_at | ACTTGTTATGCTTCCACACTACGAA | SEQ ID NO: 2286 |
| 230698_at | ACTTGGGACGTGAGTTGTCTCTCAA | SEQ ID NO: 2287 |
| 230698_at | GAGTTGTCTCTCAAAGCACAGTAGT | SEQ ID NO: 2288 |
| 230698_at | AAGCACAGCTGGGGATTGATCATGG | SEQ ID NO: 2289 |
| 230698_at | GGAGCTTGGCAGCTCTCATATCCAG | SEQ ID NO: 2290 |
| 230698_at | GCAGCTCTCATATCCAGAATAAGCC | SEQ ID NO: 2291 |
| 230698_at | ATAAGCCACTAAGACGGAACTCATC | SEQ ID NO: 2292 |
| 230698_at | ACTAAGACGGAACTCATCAATCACC | SEQ ID NO: 2293 |
| 230698_at | AATTAACTTAGCATGCAACTTACCG | SEQ ID NO: 2294 |
| 230698_at | AACTGCCATATTTACCAGATGTTTT | SEQ ID NO: 2295 |
| 230698_at | CAGATGTTTTCTTTAACCGAACTTG | SEQ ID NO: 2296 |
| 230698_at | TTAACCGAACTTGTCTGTAAATATA | SEQ ID NO: 2297 |
| 230788_at | GATAGCGAATGCACTCAGGGTCAGC | SEQ ID NO: 2298 |
| 230788_at | ACTTATTTAAATGACAGCACCTGAG | SEQ ID NO: 2299 |
| 230788_at | AGAGGAACCGTTTTACACTGGATGT | SEQ ID NO: 2300 |
| 230788_at | TACATGTCTGTTGTTGGTCATCTCT | SEQ ID NO: 2301 |
| 230788_at | GTCATCTCTCCTGTGTCTTAAATAC | SEQ ID NO: 2302 |
| 230788_at | GAGCATAGTGTTTGGGCTAGTGGGT | SEQ ID NO: 2303 |
| 230788_at | GCTAGTGGGTTTCTGACAGCCCATG | SEQ ID NO: 2304 |
| 230788_at | ACAGCCCATGGGAATGCCCTGAAAC | SEQ ID NO: 2305 |
| 230788_at | GGAATGCCCTGAAACTACTGTATCT | SEQ ID NO: 2306 |
| 230788_at | GATGTTTGTTTTCGATGAGGTTCCA | SEQ ID NO: 2307 |
| 230788_at | CGATGAGGTTCCATGTTTTGTTTTC | SEQ ID NO: 2308 |
| 232098_at | TTGGACTAGTCCTATCATAAATGGG | SEQ ID NO: 2309 |
| 232098_at | GATACTGTACCATTTGCATGTGTGC | SEQ ID NO: 2310 |
| 232098_at | TGTGTTTGTGTCTTTCTGCAGGCAC | SEQ ID NO: 2311 |
| 232098_at | TGTGTCTTTCTGCAGGCACATCTCA | SEQ ID NO: 2312 |
| 232098_at | ATCACTTTTGTGATAGGCTCACTTT | SEQ ID NO: 2313 |
| 232098_at | GGCTCACTTTTGTGAATGATCTGAG | SEQ ID NO: 2314 |
| 232098_at | GTTTGAAAGATCTAGTTGCATACAC | SEQ ID NO: 2315 |
| 232098_at | TTGCATACACAGACTCTTGGATCAA | SEQ ID NO: 2316 |
| 232098_at | CTCTGGGCTCACTTCTTAGATCAGT | SEQ ID NO: 2317 |
| 232098_at | ACTTCTTAGATCAGTCTGTGGCCAA | SEQ ID NO: 2318 |
| 232098_at | AATTCCTGGCACATCAGTTTGTCAA | SEQ ID NO: 2319 |
| 232231_at | AAGACACTTCTTCCAAACCTTGAAT | SEQ ID NO: 2320 |
| 232231_at | GATGTGTGTTTACTTCATGTTTACA | SEQ ID NO: 2321 |
| 232231_at | ATCAGCCAAAACCATAACTTACAAT | SEQ ID NO: 2322 |
| 232231_at | TTGGATATGCTTTACCATTCTTAGG | SEQ ID NO: 2323 |
| 232231_at | ACCATTCTTAGGTTTCTGTGGAACA | SEQ ID NO: 2324 |
| 232231_at | TTTTTCCAATTGCTATTGCCCAAGA | SEQ ID NO: 2325 |
| 232231_at | GCTATTGCCCAAGAATTGCTTTCCA | SEQ ID NO: 2326 |
| 232231_at | GAATTGCTTTCCATGCACATATTGT | SEQ ID NO: 2327 |
| 232231_at | TTGTAAAAATTCCGCTTTGTGCCAC | SEQ ID NO: 2328 |
| 232231_at | GCTTTGTGCCACAGGTCATGATTGT | SEQ ID NO: 2329 |
| 232231_at | AGGGACTATTTGTATTGTATGTTGC | SEQ ID NO: 2330 |
| 234994_at | ACAATCGGCTAACCTTGACATTTCT | SEQ ID NO: 2331 |
| 234994_at | CATATGCCACTATCTCGGTAGTTCA | SEQ ID NO: 2332 |
| 234994_at | TAAATTGCCTTGAAGTTTACCTTGT | SEQ ID NO: 2333 |
| 234994_at | CCTTGTGCTGGAGAGCCTTATGATA | SEQ ID NO: 2334 |
| 234994_at | GATAACTCCAAAGACTTTCTTACGG | SEQ ID NO: 2335 |
| 234994_at | TAGGATTGTGTTTCTTAGTCACTGA | SEQ ID NO: 2336 |
| 234994_at | ATACCTAAACATTTCTGAACATCAG | SEQ ID NO: 2337 |
| 234994_at | TCTGAACATCAGTATTGCAGTTGTG | SEQ ID NO: 2338 |
| 234994_at | GGAGGATACATTTGTTTGTGTTGCT | SEQ ID NO: 2339 |
| 234994_at | AAAATTCCACCTTGCATTTGCATCA | SEQ ID NO: 2340 |
| 234994_at | CCCTCAATTGAGGCAGTTTTCTTTG | SEQ ID NO: 2341 |
| 235048_at | TCAGTATTTTTATTCGCCTTCTAGA | SEQ ID NO: 2342 |
| 235048_at | ATCCACACATCACCCATTTATATTA | SEQ ID NO: 2343 |
| 235048_at | GGCTTACCTTCTGTCATCAAGTGAT | SEQ ID NO: 2344 |
| 235048_at | GTATCATCCTGGATCGTCATTTCCA | SEQ ID NO: 2345 |
| 235048_at | GTCATTTCCAAGGAACTAGCCTTTC | SEQ ID NO: 2346 |
| 235048_at | CTTTCTTTTCCTAAGCGTCTGTATG | SEQ ID NO: 2347 |
| 235048_at | GTATGTGTTCTAAAACTTCCAGTAT | SEQ ID NO: 2348 |
| 235048_at | CTGGAGTACCTATGTTTGTTTTCTT | SEQ ID NO: 2349 |
| 235048_at | GATTGTTTCCTGGTCTGTGTTTTTA | SEQ ID NO: 2350 |
| 235048_at | TTTCCTTCAGTTTTCCTCATGAAGA | SEQ ID NO: 2351 |
| 235048_at | ATCACATTGGTTGTACTCTGAAGAC | SEQ ID NO: 2352 |
| 235199_at | GCATTTTTCCAACATTGAAGGTATT | SEQ ID NO: 2353 |
| 235199_at | GTCACTAAGAGATTCATTCTTTTAT | SEQ ID NO: 2354 |
| 235199_at | AAAAGTTTCACTCTCTTTATAGTGC | SEQ ID NO: 2355 |
| 235199_at | GTGCTTCAGGATACAACTTTTTCAG | SEQ ID NO: 2356 |
| 235199_at | GATACAACTTTTTCAGGGCCTTATT | SEQ ID NO: 2357 |
| 235199_at | ACTGATTCACATGTTATTCTTCTAA | SEQ ID NO: 2358 |
| 235199_at | AGATATGGTTCCAGGCAGACCTCCT | SEQ ID NO: 2359 |
| 235199_at | TTCCAGGCAGACCTCCTTAGAGACC | SEQ ID NO: 2360 |
| 235199_at | ATTTCATTACTGTTACTGGGTGCCA | SEQ ID NO: 2361 |
| 235199_at | GGGTGCCAAGTGTCTTTCATTTGGA | SEQ ID NO: 2362 |
| 235199_at | GGAAGTGAACTTACTCCAGTTATTG | SEQ ID NO: 2363 |
| 235252_at | CCAAATCAAAACACCCTCTGTCATC | SEQ ID NO: 2364 |
| 235252_at | GCCAGTTGGAGTTTGTGCTATGCAG | SEQ ID NO: 2365 |
| 235252_at | GGATCTCATCAGCGTGCAAACCTAG | SEQ ID NO: 2366 |
| 235252_at | GTGCAAACCTAGCATCTTCTGTGGC | SEQ ID NO: 2367 |
| 235252_at | CCACAAGCCACACACTTGCTTTTTT | SEQ ID NO: 2368 |
| 235252_at | CCTGGGTTTCTGTCTAACTCGAAGT | SEQ ID NO: 2369 |
| 235252_at | TGTATCGGGTTTTTTTGCCACTGGC | SEQ ID NO: 2370 |
| 235252_at | GGCAAGAACATGCCCTCTGTGCTAA | SEQ ID NO: 2371 |
| 235252_at | ACTCGAAGTCTTGAATCCTAGCTAG | SEQ ID NO: 2372 |
| 235252_at | CTGTGCTAAGCCAGGCCTGGGTGTC | SEQ ID NO: 2373 |
| 235252_at | GTAGCAAAGTTGATCTCTCCATGTC | SEQ ID NO: 2374 |
| 235826_at | ATTTTTCTGCAGGGGTACACCCACA | SEQ ID NO: 2375 |
| 235826_at | GGGTACACCCACATCTATTGTATTA | SEQ ID NO: 2376 |
| 235826_at | TTTTCTCTGGTTGATCGGGATGCAT | SEQ ID NO: 2377 |
| 235826_at | GATCGGGATGCATTATCCACCAGAA | SEQ ID NO: 2378 |
| 235826_at | AAAACACTGTAGACGACTCACTCAC | SEQ ID NO: 2379 |
| 235826_at | ATCAAGTCTTATGAGCCAGGTGCAG | SEQ ID NO: 2380 |
| 235826_at | GAGGGTGGAGTGTGATATGATCGTC | SEQ ID NO: 2381 |
| 235826_at | AGCACTGCATCCTGGACAAGATAGG | SEQ ID NO: 2382 |
| 235826_at | ACTGCATTGTACATTCATTGAGGAC | SEQ ID NO: 2383 |
| 235826_at | GAGGACAGGGACTTTAAACTTCATT | SEQ ID NO: 2384 |
| 235826_at | ACTTCATTATATTGCTGTTGCTGTG | SEQ ID NO: 2385 |
| 236193_at | TAATACCTTAGGTTAAGGCCACATA | SEQ ID NO: 2386 |
| 236193_at | GCCTTTTCTGCGGAGGACTCTGAAG | SEQ ID NO: 2387 |
| 236193_at | GGAGGACTCTGAAGGGATACTAAAC | SEQ ID NO: 2388 |
| 236193_at | TACTTTTACCTACATTGTCTCTTAT | SEQ ID NO: 2389 |
| 236193_at | GAAAGTGTTTACTATGGACTGAATT | SEQ ID NO: 2390 |
| 236193_at | TCATATATTGAAGCCATAAACCCCA | SEQ ID NO: 2391 |
| 236193_at | TAAACCCCAATATGACTCTATTCCT | SEQ ID NO: 2392 |
| 236193_at | GACTCTATTCCTAGACAGGACTTAT | SEQ ID NO: 2393 |
| 236193_at | GGTCATTAGGATGGGTTCCTAACTG | SEQ ID NO: 2394 |
| 236193_at | ATGTTTCTTGTTAGCCATGACCCTA | SEQ ID NO: 2395 |
| 236193_at | CTTGTTAGCCATGACCCTATAAGAA | SEQ ID NO: 2396 |
| 238041_at | GACTCAGATTGTATGTCTCTAAGAA | SEQ ID NO: 2397 |
| 238041_at | TTCTCTTTCTCTTTGCAGATTTCTA | SEQ ID NO: 2398 |
| 238041_at | TGCAGATTTCTAGGCCGCTTCTGCT | SEQ ID NO: 2399 |
| 238041_at | CTTTTCTATAGTTCATGTTTTCTTT | SEQ ID NO: 2400 |
| 238041_at | AAGAATCTTAAGCTTTGGCATTAAA | SEQ ID NO: 2401 |
| 238041_at | GCTTTGGCATTAAATAGTCCTCGAT | SEQ ID NO: 2402 |
| 238041_at | AGTCCTCGATTCAAATCTAAGCTCA | SEQ ID NO: 2403 |
| 238041_at | TAAGCTCAACATCTGATTAACTTCA | SEQ ID NO: 2404 |
| 238041_at | GGAAAGCTCTTATGGTTCTTGTCAC | SEQ ID NO: 2405 |
| 238041_at | GCTCTTATGGTTCTTGTCACCTAAG | SEQ ID NO: 2406 |
| 238041_at | GAGACCATCTAGTAAATGACCTCAT | SEQ ID NO: 2407 |
| 238488_at | GAGGCACAGGTCATTCTTTTTGAAC | SEQ ID NO: 2408 |
| 238488_at | AAACTTCAGTGCCATGGACATGATT | SEQ ID NO: 2409 |
| 238488_at | GAGACTACAGCAGTGTTACCTGTGC | SEQ ID NO: 2410 |
| 238488_at | ACAACTTACTACTTCTGTTACCTTG | SEQ ID NO: 2411 |
| 238488_at | AGTGCTCTACCGAATGATGCTGCTT | SEQ ID NO: 2412 |
| 238488_at | GACATTTTGCTAGCTTTTTTCATCT | SEQ ID NO: 2413 |
| 238488_at | CTTTTTTCATCTTAGCTTGTGTTTT | SEQ ID NO: 2414 |
| 238488_at | GAATACTACAGCTTTTATCAGTCAG | SEQ ID NO: 2415 |
| 238488_at | AACTGCCTCAATTTGTAACACTTCC | SEQ ID NO: 2416 |
| 238488_at | TAACACTTCCCCAAATTCTCTAGAA | SEQ ID NO: 2417 |
| 238488_at | ATTCTCTAGAAAGTCCTGGCTTGGA | SEQ ID NO: 2418 |
| 238633_at | GCTGTGGCTTTACCTTGTTGTGGAA | SEQ ID NO: 2419 |
| 238633_at | GGAAGTTGGGTTCGGACACCAGGAT | SEQ ID NO: 2420 |
| 238633_at | ATAATAGAATCTTCCTCTCATTTCC | SEQ ID NO: 2421 |
| 238633_at | TTCCCCCAGATCCTTGACAGTATAA | SEQ ID NO: 2422 |
| 238633_at | GGAATTGCATACCTTGGTTTTCAGG | SEQ ID NO: 2423 |
| 238633_at | GGAAGTCCAGGAGTCGCGTGGATTT | SEQ ID NO: 2424 |
| 238633_at | ACTGTAATACTTCTCTTGGTACTGT | SEQ ID NO: 2425 |
| 238633_at | AGCTCAGATTGTCTAGTTGGGCACT | SEQ ID NO: 2426 |
| 238633_at | GGGCACTGACTTTCAGCACATTGTC | SEQ ID NO: 2427 |
| 238633_at | GTCTCATGAGACACTACCTCTTAAT | SEQ ID NO: 2428 |
| 238633_at | GAGTAGCATGGCCATTTGTTTATTT | SEQ ID NO: 2429 |
| 238974_at | CATTTATTTTCACATGATTAACTGA | SEQ ID NO: 2430 |
| 238974_at | AAATCTAGGTTGTCTATCCAGTATG | SEQ ID NO: 2431 |
| 238974_at | GTCTATCCAGTATGTGAATGCTTAA | SEQ ID NO: 2432 |
| 238974_at | GAGTAAGTCACCAGGTACACAAAAC | SEQ ID NO: 2433 |
| 238974_at | ATATATTCAAGTTGATCCATATTCA | SEQ ID NO: 2434 |
| 238974_at | AATACTTCAGATTGGTCCTTTGTCC | SEQ ID NO: 2435 |
| 238974_at | TGGTCCTTTGTCCACATTTGTTTAA | SEQ ID NO: 2436 |
| 238974_at | ATCCTTGGCTAAATTCACATGTATC | SEQ ID NO: 2437 |
| 238974_at | TATACTTTTGGATTGTGCCTTTGTC | SEQ ID NO: 2438 |
| 238974_at | TTTTGGATTGTGCCTTTGTCATGAG | SEQ ID NO: 2439 |
| 238974_at | AGCTGAGTTACTGAATTCTATAAGG | SEQ ID NO: 2440 |
| 239835_at | GTATGTAGCACTTTCCTATATATTT | SEQ ID NO: 2441 |
| 239835_at | TGAAAACTGGACTGGGTATAACTAT | SEQ ID NO: 2442 |
| 239835_at | AAAAGGCACAATGGTACTACAGAAT | SEQ ID NO: 2443 |
| 239835_at | GTTTTCTGTTCTACAAAGTTGATGC | SEQ ID NO: 2444 |
| 239835_at | GAATCAGATTCCCTATGTAAAGCAG | SEQ ID NO: 2445 |
| 239835_at | GGAATTCAATGTTCAGTGCTCAGGT | SEQ ID NO: 2446 |
| 239835_at | TGTAGTAAGTACTGTAGTCCTGTGG | SEQ ID NO: 2447 |
| 239835_at | GTAGTCCTGTGGGGGCAAATGTGTA | SEQ ID NO: 2448 |
| 239835_at | GGTCTAACATAATGCCAGTTCCACT | SEQ ID NO: 2449 |
| 239835_at | ATGCCAGTTCCACTTTAACTTTGTT | SEQ ID NO: 2450 |
| 239835_at | GAAGAATGTATGTAGCACTTTCCTA | SEQ ID NO: 2451 |
| 240165_at | AAGGAAGGTCAGTCAGTGAATGGGA | SEQ ID NO: 2452 |
| 240165_at | GAAAGGGAGCTCCTCTAGCATCAAA | SEQ ID NO: 2453 |
| 240165_at | GAGCTCCTCTAGCATCAAACTGTCT | SEQ ID NO: 2454 |
| 240165_at | CAAACTGTCTGCATGTCGAGTCTCA | SEQ ID NO: 2455 |
| 240165_at | CTGCATGTCGAGTCTCAGAAAAACA | SEQ ID NO: 2456 |
| 240165_at | AACAAGGATTCGTCAGTCAACCCCT | SEQ ID NO: 2457 |
| 240165_at | CCCCTTTCTGCATGCACAGTGGATT | SEQ ID NO: 2458 |
| 240165_at | GCATGCACAGTGGATTTAGGGTAAA | SEQ ID NO: 2459 |
| 240165_at | TAAAGTTTATGTTACCCTGTCTTTG | SEQ ID NO: 2460 |
| 240165_at | AATGACTCATGAACTTAAGGTACTT | SEQ ID NO: 2461 |
| 240165_at | CCATAGCGGAGAACTACTGAGTTAA | SEQ ID NO: 2462 |
| 243010_at | CTTAGCCTGACAGTGTCCTGTTCTC | SEQ ID NO: 2463 |
| 243010_at | GAAATACACCCACTCTCTTGGAATA | SEQ ID NO: 2464 |
| 243010_at | ATGACGTACCACTCAGTTGGACCCT | SEQ ID NO: 2465 |
| 243010_at | GACCCTCAAGAGTCACTGCTTTGTC | SEQ ID NO: 2466 |
| 243010_at | CGCACGCTTCCATTTGATGCATTTG | SEQ ID NO: 2467 |
| 243010_at | ATGTCATTGTCCTTGAGACCCTACA | SEQ ID NO: 2468 |
| 243010_at | GAGACCCTACATGTGCAGTTTGGCT | SEQ ID NO: 2469 |
| 243010_at | TTTCCTGCAGGCTTTTCCATGAGTA | SEQ ID NO: 2470 |
| 243010_at | GAACAAATCTGTATGGCTTTTCCCC | SEQ ID NO: 2471 |
| 243010_at | GTGAACTTGTCCTAGTATGCTTGCC | SEQ ID NO: 2472 |
| 243010_at | CTTGCCTCACAAACGTTTTAGCCAT | SEQ ID NO: 2473 |
| 243092_at | GGTGATGTTCTCTAGCCAAATTCGA | SEQ ID NO: 2474 |
| 243092_at | AGCAGTTTCGCTTATTTGATTATTC | SEQ ID NO: 2475 |
| 243092_at | ACGCATTACGTGTACCAGAAACTGT | SEQ ID NO: 2476 |
| 243092_at | GGTACACTTAACTGTGGAGCTGGGG | SEQ ID NO: 2477 |
| 243092_at | ACATGCCGCCTTAAGTGAGTTCAGA | SEQ ID NO: 2478 |
| 243092_at | GAGTTCAGATGGCTTATCTTCCGGT | SEQ ID NO: 2479 |
| 243092_at | GAGGCATCAAGTACACAGGTCCGTT | SEQ ID NO: 2480 |
| 243092_at | ACAGGTCCGTTGTAAACCAGTGTCT | SEQ ID NO: 2481 |
| 243092_at | AACCAGTGTCTTAAGTGCTAACCTT | SEQ ID NO: 2482 |
| 243092_at | TGCTAACCTTATCACATTTGCTATT | SEQ ID NO: 2483 |
| 243092_at | TGCCTTGTCTGTACACCTGGATTAA | SEQ ID NO: 2484 |
| 243835_at | GTAATTGTCCAAATGTAATGCTGCT | SEQ ID NO: 2485 |
| 243835_at | GCTATGTATATTATTTGGGTTCCAG | SEQ ID NO: 2486 |
| 243835_at | TTCTCAAAACACTCAGTGTCCTTAC | SEQ ID NO: 2487 |
| 243835_at | GTGTCCTTACAACTGCAGCTAAAAT | SEQ ID NO: 2488 |
| 243835_at | CAACCTCTCCTTGAATGTAGATACA | SEQ ID NO: 2489 |
| 243835_at | GGTTTTGCAGTCAATTCTGAATGGA | SEQ ID NO: 2490 |
| 243835_at | ATGTGGCTTCAGATCATTTGAACGA | SEQ ID NO: 2491 |
| 243835_at | GCAACATTATCTCTCTCTAATCTGC | SEQ ID NO: 2492 |
| 243835_at | ATATTCCCTAGATTGTGTTGCCACT | SEQ ID NO: 2493 |
| 243835_at | TTGTGTTGCCACTGTATTGATTCTG | SEQ ID NO: 2494 |
| 243835_at | AATTTGGCTTGTTTATGCGTGATTT | SEQ ID NO: 2495 |
| 244110_at | AAAATTGCATTGGCCAACTTGGAGG | SEQ ID NO: 2496 |
| 244110_at | GGCCAACTTGGAGGCTTCAGTGTTA | SEQ ID NO: 2497 |
| 244110_at | AGAAGAATGTTCACTTTTGTCATCT | SEQ ID NO: 2498 |
| 244110_at | GTCATCTAATTTTACACTGCTCCTT | SEQ ID NO: 2499 |
| 244110_at | ACACTGCTCCTTCAGCAAACTGACT | SEQ ID NO: 2500 |
| 244110_at | GAGAGATAACCCTGTTTACCTTTAG | SEQ ID NO: 2501 |
| 244110_at | AGTTGTGGATTCCTCAGTCTTACTC | SEQ ID NO: 2502 |
| 244110_at | GTCTTACTCCCATTACTATTGGTCA | SEQ ID NO: 2503 |
| 244110_at | TATTGGTCATTCAACAGCCCATCTT | SEQ ID NO: 2504 |
| 244110_at | GTAACCTGACTTTTGCGCCAGAATA | SEQ ID NO: 2505 |
| 244110_at | AGTTAACCACTTAAACTTGTCATAT | SEQ ID NO: 2506 |
| 244519_at | TTAGAAAACTACTCGGATGCTCCAA | SEQ ID NO: 2507 |
| 244519_at | CTACTCGGATGCTCCAATGACACCA | SEQ ID NO: 2508 |
| 244519_at | CAATGACACCAAAACAGATTCTGCA | SEQ ID NO: 2509 |
| 244519_at | TCTCGCATGCCTCAATGCTATGCTA | SEQ ID NO: 2510 |
| 244519_at | CGCATGCCTCAATGCTATGCTACAT | SEQ ID NO: 2511 |
| 244519_at | GCCTCAATGCTATGCTACATTCCAA | SEQ ID NO: 2512 |
| 244519_at | CAATGCTATGCTACATTCCAATTCA | SEQ ID NO: 2513 |
| 244519_at | TGTTTTATAAACTGCCTGGCCGAAT | SEQ ID NO: 2514 |
| 244519_at | ATAAACTGCCTGGCCGAATCAGCCT | SEQ ID NO: 2515 |
| 244519_at | ATCAGCCTTTTCACGCTCAAGGTGT | SEQ ID NO: 2516 |
| 244519_at | GCCTTTTCACGCTCAAGGTGTGAGC | SEQ ID NO: 2517 |
| 60084_at | GTTATAATCTCTTCCTAGCTAATGG | SEQ ID NO: 2518 |
| 60084_at | CTCTTCCTAGCTAATGGGCTTACTC | SEQ ID NO: 2519 |
| 60084_at | CTTCCTAGCTAATGGGCTTACTCAA | SEQ ID NO: 2520 |
| 60084_at | TAGCTAATGGGCTTACTCAAAGATT | SEQ ID NO: 2521 |
| 60084_at | TGGGCTTACTCAAAGATTCACCACC | SEQ ID NO: 2522 |
| 60084_at | CTAGCAATGATATTCTCAGTTGTTT | SEQ ID NO: 2523 |
| 60084_at | AGCAATGATATTCTCAGTTGTTTCT | SEQ ID NO: 2524 |
| 60084_at | GCAATGATATTCTCAGTTGTTTCTC | SEQ ID NO: 2525 |
| 60084_at | CTCAGTTGTTTCTCTCTTGTGGTGC | SEQ ID NO: 2526 |
| 60084_at | TTCTCTCTTGTGGTGCAGAGTTGCA | SEQ ID NO: 2527 |
| 60084_at | TCTCTTGTGGTGCAGAGTTGCATTG | SEQ ID NO: 2528 |
| 60084_at | CTCTTGTGGTGCAGAGTTGCATTGG | SEQ ID NO: 2529 |
| 60084_at | TGCAGAGTTGCATTGGGTTTTCTAC | SEQ ID NO: 2530 |
| 60084_at | TGCATTGGGTTTTCTACATTTTCCC | SEQ ID NO: 2531 |
| 60084_at | GCATTGGGTTTTCTACATTTTCCCA | SEQ ID NO: 2532 |
| 60084_at | CCCACTGAGTCTTCCCTGTTGTAAA | SEQ ID NO: 2533 |

TABLE 18

| Probe Set ID | Probe sequence | Sequence ID No. |
|---|---|---|
| 201018_at | GCTTCAGGTTCTTCACCTCTAAGAT | SEQ ID NO: 1012 |
| 201018_at | GGGGATGATGAAAACAGTACCTGTC | SEQ ID NO: 1013 |
| 201018_at | GTACCTGTCATGCAGAATTGTTGGG | SEQ ID NO: 1014 |
| 201018_at | TGCTCTTTTCACTTGATATCCAGTA | SEQ ID NO: 1015 |
| 201018_at | GAAGGTGCATGTCTTCTGTATTCTG | SEQ ID NO: 1016 |
| 201018_at | CCCATTTCTTTTGCGTGCAGTCTTT | SEQ ID NO: 1017 |
| 201018_at | TTGCGTGCAGTCTTTGATTCGTACA | SEQ ID NO: 1018 |
| 201018_at | GAAATTGCTACCAAACTCATTTAAT | SEQ ID NO: 1019 |
| 201018_at | ATACCAACTGTTCTATATTTCTTTA | SEQ ID NO: 1020 |
| 201018_at | ATCTTCAGTGATTCCTTTTACTATA | SEQ ID NO: 1021 |
| 201018_at | AGGTTTCCTTTCCCATCATATGGAA | SEQ ID NO: 1022 |
| 201080_at | CCCATTCAGACAACTGTTCCCCAAT | SEQ ID NO: 1023 |
| 201080_at | CTACCAGCCATCTGCAGGGGTCAGT | SEQ ID NO: 1024 |
| 201080_at | GTGCCACTTATGAAGAGTGCCCCAT | SEQ ID NO: 1025 |
| 201080_at | AAAAGGAGACTCAGCTGTCCCTTGG | SEQ ID NO: 1026 |
| 201080_at | CTTGTGCCAGTATCCCAGGGCAGAA | SEQ ID NO: 1027 |
| 201080_at | CCTTGCGCAGAGCCACTGTGAGAGG | SEQ ID NO: 1028 |
| 201080_at | TGAGAGGCGGTGGGAGCCAACACCC | SEQ ID NO: 1029 |
| 201080_at | ATTAAGTTCATATCCACCTTTTGGG | SEQ ID NO: 1030 |
| 201080_at | CCAAGTGTGTGACTTCTCCATATCC | SEQ ID NO: 1031 |
| 201080_at | TGGGAATTTTCAATCCCCTGTGCTT | SEQ ID NO: 1032 |
| 201080_at | TGCTTGTCTAACGTCTGCTTTAAAA | SEQ ID NO: 1033 |
| 202599_s_at | ATTTAAGTTGTGATTACCTGCTGCA | SEQ ID NO: 1034 |
| 202599_s_at | AAGTGGCATGGGGGACCCTGTGCAT | SEQ ID NO: 1035 |
| 202599_s_at | GACCCTGTGCATCTGTGCATTTGGC | SEQ ID NO: 1036 |
| 202599_s_at | TCCATTTCTGGACATGACGTCTGTG | SEQ ID NO: 1037 |
| 202599_s_at | GACGTCTGTGGTTTAAGCTTTGTGA | SEQ ID NO: 1038 |
| 202599_s_at | AATGTGCTTTGATTCGAAGGGTCTT | SEQ ID NO: 1039 |
| 202599_s_at | TAATCGTCAACCACTTTTAAACATA | SEQ ID NO: 1040 |
| 202599_s_at | AGAATTCACACAACTACTTTCATGA | SEQ ID NO: 1041 |
| 202599_s_at | ATTCCAAGAGTATCCCAGTATTAGC | SEQ ID NO: 1042 |
| 202599_s_at | ATATAGGCACATTACCATTCATAGT | SEQ ID NO: 1043 |
| 202599_s_at | AATTTGATGCGATCTGCTCAGTAAT | SEQ ID NO: 1044 |
| 203106_s_at | TATTATTGAATGTACCCCTCAGCCT | SEQ ID NO: 1045 |
| 203106_s_at | AGCATTTCCTTATCCCAAGACTAGT | SEQ ID NO: 1046 |
| 203106_s_at | CCAAGACTAGTGTGCTTTCTGCTAC | SEQ ID NO: 1047 |
| 203106_s_at | CTTTCTGCTACACTGCTAGTTTTCA | SEQ ID NO: 1048 |
| 203106_s_at | GCTACACTGCTAGTTTTCAGTTTTG | SEQ ID NO: 1049 |
| 203106_s_at | AACATTACCAATTTACAGATTCAGT | SEQ ID NO: 1050 |
| 203106_s_at | TTACATTTACATTAATCCTCACTTA | SEQ ID NO: 1051 |
| 203106_s_at | TGAGCAAGCTCATTTCCAGAAAAGT | SEQ ID NO: 1052 |
| 203106_s_at | TTTCAGTGAAGTCATTTTGCTTCAG | SEQ ID NO: 1053 |
| 203106_s_at | ATTATCCTAGTTACCAAGTCCTATT | SEQ ID NO: 1054 |
| 203106_s_at | TATGTTCGTTTATCATTTCAGAAAT | SEQ ID NO: 1055 |
| 204837_at | ATTCATGCTCTGCTAGTCTATGCCT | SEQ ID NO: 1056 |
| 204837_at | GTCTATGCCTGCAACTCCAAATGTT | SEQ ID NO: 1057 |
| 204837_at | CAGTATTTCCCACCTACATTTCTGT | SEQ ID NO: 1058 |
| 204837_at | TATGACCGAGTCTAGTTTTTCTTTA | SEQ ID NO: 1059 |
| 204837_at | AAATACTTTTCATCACCAATTGCCC | SEQ ID NO: 1060 |
| 204837_at | TGCTTCCTCAGCCTTGTAGCAAAGG | SEQ ID NO: 1061 |
| 204837_at | AGCAAAGGCTACACAGCAGCCCACA | SEQ ID NO: 1062 |
| 204837_at | GCCCACAGTCCACAGTCTTTTTGGG | SEQ ID NO: 1063 |
| 204837_at | CTGCCACCTTCTTTAAGCTCAGTTT | SEQ ID NO: 1064 |
| 204837_at | TTTGACTTACTTTCTTTGCTGTAGT | SEQ ID NO: 1065 |
| 204837_at | TTCTCGTAGCTCTGCGTTGTGTGAA | SEQ ID NO: 1066 |
| 205094_at | AGAAAGCATTTACCTGCCTGTCTGT | SEQ ID NO: 1067 |
| 205094_at | GCATTTACCTGCCTGTCTGTAAGGT | SEQ ID NO: 1068 |
| 205094_at | GTGGAAATTTCATCAGTTTGCAAAC | SEQ ID NO: 1069 |
| 205094_at | AAAAAGCTCCTTCCATATACTGTGA | SEQ ID NO: 1070 |
| 205094_at | GAGACATTTGTTAAGTGACATCTAT | SEQ ID NO: 1071 |
| 205094_at | GACATCTATTGTTTATCAGCTTTTA | SEQ ID NO: 1072 |
| 205094_at | GGATATTCCTTTATGAGCTCTCCAT | SEQ ID NO: 1073 |
| 205094_at | AGCTCTCCATATCCTTCTTGAGAAA | SEQ ID NO: 1074 |
| 205094_at | GAGAGTAGTCTGAAGATTCCTGTGT | SEQ ID NO: 1075 |
| 205094_at | AATAAGTTCTTTCTGCTTGCTGCTA | SEQ ID NO: 1076 |
| 205094_at | TCTGCTTGCTGCTAAGAGTTTGCTA | SEQ ID NO: 1077 |
| 205608_s_at | AGAGCAGCCTGATCTTACACGGTGC | SEQ ID NO: 1078 |
| 205608_s_at | GTGCAAATGTGCCCTCATGTTAACA | SEQ ID NO: 1079 |
| 205608_s_at | TCAAAGGGCCCAGTTACTCCTTACG | SEQ ID NO: 1080 |
| 205608_s_at | TACTCCTTACGTTCCACAACTATGA | SEQ ID NO: 1081 |
| 205608_s_at | GCAAACAATATTGTCTCCCTTCCAG | SEQ ID NO: 1082 |
| 205608_s_at | GGTTCTTGACCGTGAATCTGGAGCC | SEQ ID NO: 1083 |
| 205608_s_at | AATCTGGAGCCGTTTGAGTTCACAA | SEQ ID NO: 1084 |
| 205608_s_at | GTCTCTACTTGGGGTGACAGTGCTC | SEQ ID NO: 1085 |
| 205608_s_at | TGCTCACGTGGCTCGACTATAGAAA | SEQ ID NO: 1086 |
| 205608_s_at | AAAACTCCACTGACTGTCGGGCTTT | SEQ ID NO: 1087 |
| 205608_s_at | GCTTGCTGTGCTTCAAACTACTACT | SEQ ID NO: 1088 |
| 205702_at | GAATCCCTTAATCTACAATATCACA | SEQ ID NO: 1089 |
| 205702_at | TCCTTTCTGCTGTCTCAGGTGTTAT | SEQ ID NO: 1090 |
| 205702_at | TGAGTTAAATGCCTGGACTCTCCCC | SEQ ID NO: 1091 |
| 205702_at | TCCCCTGGCTGGTATCAAAACTTAC | SEQ ID NO: 1092 |
| 205702_at | AAACCAGTGAGATACCCACCTGCTT | SEQ ID NO: 1093 |
| 205702_at | CCACCTGCTTGTTCACATGCACAGG | SEQ ID NO: 1094 |
| 205702_at | GTTCACATGCACAGGTGCTCTCAGC | SEQ ID NO: 1095 |
| 205702_at | GCTCTCAGCTCTGCAAAGCGAATGA | SEQ ID NO: 1096 |
| 205702_at | GGAGGAGCAAGTCCTTTTCCAACTG | SEQ ID NO: 1097 |
| 205702_at | TTTTCCAACTGGGTGTGCATGCTAA | SEQ ID NO: 1098 |
| 205702_at | GATAGTTTAGCTTCAGTACTGTGAC | SEQ ID NO: 1099 |
| 206874_s_at | GAAGGGTCTCTGATTTCTTGAGCAT | SEQ ID NO: 1100 |
| 206874_s_at | GAAGCCAAATTCTGTCCAAGTATTA | SEQ ID NO: 1101 |
| 206874_s_at | GAGAGTTCCAGTTCTAATAGTCTTT | SEQ ID NO: 1102 |
| 206874_s_at | AATGGCTGTATTGTTGCTATTCCGT | SEQ ID NO: 1103 |
| 206874_s_at | GCTATTCCGTTGCTGACATGTTTTT | SEQ ID NO: 1104 |
| 206874_s_at | AAAGCTTTAACATTCCTGCTACTAA | SEQ ID NO: 1105 |
| 206874_s_at | GCGGAGAGTGTTTGCCAGGTTTCAA | SEQ ID NO: 1106 |
| 206874_s_at | AGGTTTCAATGTGGGCTGCAGCTTT | SEQ ID NO: 1107 |
| 206874_s_at | CTCCTTCTCTGGTTTGCAGTGTAAT | SEQ ID NO: 1108 |
| 206874_s_at | GATTATGCCTCTTATCTACTTGAGA | SEQ ID NO: 1109 |
| 206874_s_at | GAGAGCAACATGTCTTTTCAATCAT | SEQ ID NO: 1110 |
| 206945_at | TTCTCTTGTGCTTCTTGGAGTCTGT | SEQ ID NO: 1111 |
| 206945_at | GTGGCTTGGCATTTCTGTCATACAA | SEQ ID NO: 1112 |
| 206945_at | TACAAGTACTGCAAGCGCTCTAAGC | SEQ ID NO: 1113 |
| 206945_at | AACAGGAATTGAGCCCGGTGTCTTC | SEQ ID NO: 1114 |
| 206945_at | GTTACCACCTCAAGTTCTATGAAGC | SEQ ID NO: 1115 |
| 206945_at | GCCACCAAACACCTTAGGGTCTTAG | SEQ ID NO: 1116 |
| 206945_at | AGACTCTGCTGATACTGGACTTCTC | SEQ ID NO: 1117 |
| 206945_at | AAAGTCCTGCTGCACCGTTAGAGAT | SEQ ID NO: 1118 |
| 206945_at | TCTCCATCTTGCTCCAGTATCAGAG | SEQ ID NO: 1119 |
| 206945_at | GATACTGGTCTAGTGGGTCTGTGAA | SEQ ID NO: 1120 |
| 206945_at | TAGACTGCAATATCATCTCCTGCCC | SEQ ID NO: 1121 |
| 207737_at | GAAGTTTTCAGTAATTGTGACTTTT | SEQ ID NO: 1122 |
| 207737_at | GGAAGTACATCCAGTAAACAATGCC | SEQ ID NO: 1123 |
| 207737_at | TAAACAATGCCATGTACATTCCCCC | SEQ ID NO: 1124 |
| 207737_at | TCCCCATTTGCTGTCCAGAGTGTGA | SEQ ID NO: 1125 |
| 207737_at | GAGTGTGACCACAGTTAACGGTTAA | SEQ ID NO: 1126 |
| 207737_at | GTTAACGGTTAATGTGCATCTTTTA | SEQ ID NO: 1127 |
| 207737_at | GCATCTTTTATGTACTTAACATGTC | SEQ ID NO: 1128 |
| 207737_at | GAACTTCCATGTTAGTATGTGCAGC | SEQ ID NO: 1129 |
| 207737_at | GTGCAGCTGTAACACATTCTTTTTT | SEQ ID NO: 1130 |
| 207737_at | ATTCTTTTTTTAGTAGCCACATAGT | SEQ ID NO: 1131 |
| 207737_at | AAAATACATTACCCATTTCCTGCTG | SEQ ID NO: 1132 |
| 207968_s_at | AGTGCAGACCTGTCATCTCTGTCTG | SEQ ID NO: 1133 |
| 207968_s_at | TCTCTGTCTGGGTTTAACACCGCCA | SEQ ID NO: 1134 |
| 207968_s_at | CGCTCTTCACCTTGGTTCAGTAACT | SEQ ID NO: 1135 |
| 207968_s_at | GCAACACCTACATAACATGCCACCA | SEQ ID NO: 1136 |
| 207968_s_at | CCATCTGCCCTCAGTCAGTTGGGAG | SEQ ID NO: 1137 |
| 207968_s_at | CTTGCACTAGCACTCATTTATCTCA | SEQ ID NO: 1138 |
| 207968_s_at | CCTTCTACTCAAAGCCTCAACATCA | SEQ ID NO: 1139 |
| 207968_s_at | GGCGGGGAGATCTCCTGTTGACAGC | SEQ ID NO: 1140 |
| 207968_s_at | GCAGCTGTAGCAGTTCGTACGACGG | SEQ ID NO: 1141 |
| 207968_s_at | GGATCACCGGAACGAATTCCACTCC | SEQ ID NO: 1142 |
| 207968_s_at | CAAGCGCATGCGACTTTCTGAAGGA | SEQ ID NO: 1143 |
| 208634_s_at | TAAACTGATTTGTTGCTCCCTATCC | SEQ ID NO: 1144 |
| 208634_s_at | ACCAGTAACTCTTGTGTTCACCAGG | SEQ ID NO: 1145 |
| 208634_s_at | GGGATAGGCTCGTTGGTGACATTGT | SEQ ID NO: 1146 |
| 208634_s_at | TAAATGGTCGATCAACTTCCCACAA | SEQ ID NO: 1147 |
| 208634_s_at | TGAATTCCACGAGCCTGTTCTGAAA | SEQ ID NO: 1148 |
| 208634_s_at | AAGACAAACACGTGCTCGTCCTTTA | SEQ ID NO: 1149 |
| 208634_s_at | TAATGGAGTTCACCAGCACACTTGT | SEQ ID NO: 1150 |
| 208634_s_at | AGCACACTTGTTAACCAGTCCTGTT | SEQ ID NO: 1151 |
| 208634_s_at | TTTGCTTTCGTCTTTTTTTGTGCGT | SEQ ID NO: 1152 |
| 208634_s_at | ATGAAAAGGGGCTGTCTGGGGCTCC | SEQ ID NO: 1153 |
| 208634_s_at | AGCTCCGACCATGTTGCTGTGTGAT | SEQ ID NO: 1154 |
| 209200_at | GGAGCAATCCAAGCCACATATCTTC | SEQ ID NO: 1155 |
| 209200_at | ATCTGGTATTGCATTTTGCCTTCCC | SEQ ID NO: 1156 |
| 209200_at | CTTCCCTGTTCATACCTCAAATTGA | SEQ ID NO: 1157 |
| 209200_at | AAGTGACGGATTCTGTTGTGGTTTG | SEQ ID NO: 1158 |
| 209200_at | GAATGCAGTACCAGTGTTCTCTTCG | SEQ ID NO: 1159 |
| 209200_at | GTAGACCTGGGTCACTGTAGGCATA | SEQ ID NO: 1160 |
| 209200_at | GGACTTGGATTGCTTCAGATGGTTT | SEQ ID NO: 1161 |
| 209200_at | TCTTTTCCTGGGGACTTGTTTCCAT | SEQ ID NO: 1162 |
| 209200_at | ATAGAGGCTCACAGCGGCATAAGCT | SEQ ID NO: 1163 |
| 209200_at | TGGACTTTGTCGCCACTAGATGACA | SEQ ID NO: 1164 |
| 209200_at | CCACATCTGTGTATCTCAAGGGACT | SEQ ID NO: 1165 |
| 209425_at | CTACCTCACTAGTAGTTCACGTGAT | SEQ ID NO: 1166 |
| 209425_at | GTAGTTCACGTGATGTCTGACAGAT | SEQ ID NO: 1167 |
| 209425_at | TGAGATACTCTTGTGAGGTCACTCT | SEQ ID NO: 1168 |
| 209425_at | CTTGTGAGGTCACTCTAATGCCCTG | SEQ ID NO: 1169 |
| 209425_at | TAAGCTTTCATATTCTAGCCTTCAG | SEQ ID NO: 1170 |
| 209425_at | CATATTCTAGCCTTCAGTCTTGTTC | SEQ ID NO: 1171 |
| 209425_at | CAGTCTTGTTCTTCAACCATTTTTA | SEQ ID NO: 1172 |
| 209425_at | TTAGGAACTTTCCCATAAGGTTATG | SEQ ID NO: 1173 |
| 209425_at | ATAAGGTTATGTTTTCCAGCCCAGG | SEQ ID NO: 1174 |
| 209425_at | TCCAGCCCAGGCATGGAGGATCACT | SEQ ID NO: 1175 |
| 209425_at | GGCCACAGTGAATTAGGATTGCACC | SEQ ID NO: 1176 |
| 210132_at | GGGGAGGGGACTAGATGGGCAAGGG | SEQ ID NO: 1177 |
| 210132_at | TGGGCAAGGGGCAGCACTGCCTGCT | SEQ ID NO: 1178 |
| 210132_at | TTCCTTCCCCTGTTTACAGCAATAA | SEQ ID NO: 1179 |
| 210132_at | TTACAGCAATAAGCACGTCCTCCTC | SEQ ID NO: 1180 |
| 210132_at | ACTCCCACTTCCAGGATTGTGGTTT | SEQ ID NO: 1181 |
| 210132_at | CAAGTTTACAAGTAGACACCCCTGG | SEQ ID NO: 1182 |
| 210132_at | AAGGGGTGGGCATTGGGGTGCCAGG | SEQ ID NO: 1183 |
| 210132_at | CCAGGCAGGCATGTACAGACTCTAT | SEQ ID NO: 1184 |
| 210132_at | GACAGGACCTATGCAACGCACAGAC | SEQ ID NO: 1185 |
| 210132_at | CGCACAGACACTTTTGGAGACCGTA | SEQ ID NO: 1186 |
| 210132_at | CTTTCATACTCTGCTCTTAGTCTAA | SEQ ID NO: 1187 |
| 211255_x_at | GACTCCCTCAAGCAAGCTGTGGGGC | SEQ ID NO: 1188 |
| 211255_x_at | CTCCCCACTATCCTGTGGTGTGTTG | SEQ ID NO: 1189 |
| 211255_x_at | CTTCGGGTCCTCAGATGTGTAGCAA | SEQ ID NO: 1190 |
| 211255_x_at | GACACTGGGCAGTTTATGCTATTCA | SEQ ID NO: 1191 |
| 211255_x_at | GTACATCAGACTGCGGGTTCGGGCT | SEQ ID NO: 1192 |
| 211255_x_at | ACTGCCAGCATGAGACTGCTCTGCA | SEQ ID NO: 1193 |
| 211255_x_at | TCTGCAGGGCAATGTCTTCTCTAAC | SEQ ID NO: 1194 |
| 211255_x_at | GTTTGAGCGCTTTAACCAGGCCAAC | SEQ ID NO: 1195 |
| 211255_x_at | ACATCAAGTTCTCTGAGCTCACCTA | SEQ ID NO: 1196 |
| 211255_x_at | TACCTCGATGCATTCTGGCGTGACT | SEQ ID NO: 1197 |
| 211255_x_at | GGCGTGACTACATCAATGGCTCTTT | SEQ ID NO: 1198 |
| 211877_s_at | GCTGTGGCGCTGGCATAAGTCACGC | SEQ ID NO: 1199 |
| 211877_s_at | CCTGCTGCAGGCTTCTGAAGGCGGG | SEQ ID NO: 1200 |
| 211877_s_at | AAGGCGGGTTGGCAGGTATGCCCAC | SEQ ID NO: 1201 |
| 211877_s_at | GTCACATTTTGTAGGCGTGGACGGG | SEQ ID NO: 1202 |
| 211877_s_at | GTAGGCGTGGACGGGGTACAGGCTT | SEQ ID NO: 1203 |
| 211877_s_at | TCTCTCTCATTGCGGACTCGCAGAA | SEQ ID NO: 1204 |
| 211877_s_at | CGCAGAAGAGTCACCTGATTTTCCC | SEQ ID NO: 1205 |
| 211877_s_at | GAAAAGCGAGCCACTCTTGATAGCT | SEQ ID NO: 1206 |
| 211877_s_at | GCCACTCTTGATAGCTGAAGACTCA | SEQ ID NO: 1207 |
| 211877_s_at | GAAGACTCAGCTATCATTTTAGGCA | SEQ ID NO: 1208 |
| 211877_s_at | GGCAAATGTGACCCGACAAGTAATC | SEQ ID NO: 1209 |
| 212397_at | GAAGTGACTGTTGTACCATGGTTGT | SEQ ID NO: 1210 |
| 212397_at | GTACCATGGTTGTGCACATGCTTCA | SEQ ID NO: 1211 |
| 212397_at | GTGCACATGCTTCAGAATCCTATGG | SEQ ID NO: 1212 |
| 212397_at | GAATATTCCTACTTGCAGTACATCA | SEQ ID NO: 1213 |
| 212397_at | GAATGGATGGTGGACCCTACTATTC | SEQ ID NO: 1214 |
| 212397_at | GTGGACCCTACTATTCATGTTTTGA | SEQ ID NO: 1215 |
| 212397_at | TGTGCACTACCATAGCTACATCAGT | SEQ ID NO: 1216 |
| 212397_at | ATATTTTGCTGTTTATGATCTATTT | SEQ ID NO: 1217 |
| 212397_at | TTTAAGGCTGTGTGAATTTTTCTAA | SEQ ID NO: 1218 |
| 212397_at | TAGCAGTCGCGAGCACATGTTCATA | SEQ ID NO: 1219 |
| 212397_at | TCCCAGTAGGCTTTTACCATTAGCA | SEQ ID NO: 1220 |
| 212851_at | AATTCAAATTGCACCTCTTTTCTTA | SEQ ID NO: 1221 |
| 212851_at | TTTGCATTCTTCTAGCCAGTGATTG | SEQ ID NO: 1222 |
| 212851_at | ATGCTTTCTTTGCCACTCTAAGTAA | SEQ ID NO: 1223 |
| 212851_at | GCTGGCTGTTTATAACTGCATCGCA | SEQ ID NO: 1224 |
| 212851_at | GCATCGCACTTCTAGTTGTGGCTTG | SEQ ID NO: 1225 |
| 212851_at | TGTTTCATGCTAGGCTTTTCCTGGC | SEQ ID NO: 1226 |
| 212851_at | TTTCCTGGCAGCATGTCCATTGCAG | SEQ ID NO: 1227 |
| 212851_at | GAAACCACCAGCATTGAGCTAACCC | SEQ ID NO: 1228 |
| 212851_at | GCTAACCCAGTACATGCTAGGACCT | SEQ ID NO: 1229 |
| 212851_at | TGTCCTAGAGGGGCCACTTTTCATT | SEQ ID NO: 1230 |
| 212851_at | GGCCACTTTTCATTACCTGAGTTAT | SEQ ID NO: 1231 |
| 213313_at | GTGATATGCTGACAGGCTGACACGC | SEQ ID NO: 1232 |
| 213313_at | GCTGACACGCAGATGGTTTTGTCCT | SEQ ID NO: 1233 |
| 213313_at | CTGCGTTCAGTGTTGAGGCGGCTGC | SEQ ID NO: 1234 |
| 213313_at | GCGGCTGCTTACAAGAGGCACTGGT | SEQ ID NO: 1235 |
| 213313_at | GACACTCGGGTTGTTTTGTAGCTCT | SEQ ID NO: 1236 |
| 213313_at | GTAGCTCTTTTTCTTATTGGCTGTA | SEQ ID NO: 1237 |
| 213313_at | TATTGGCTGTACTAACGCTTGCTGA | SEQ ID NO: 1238 |
| 213313_at | AACGCTTGCTGAGGTTATCTGTAAT | SEQ ID NO: 1239 |
| 213313_at | GCTTTCTGTGTCTTTCTTGTTCAGT | SEQ ID NO: 1240 |
| 213313_at | GCTAGGTGTGTGGACATTGTGCTAA | SEQ ID NO: 1241 |
| 213313_at | GTGCTAAGGTAGTTTCAGTGTGTCA | SEQ ID NO: 1242 |
| 213639_s_at | TGTCTGGAATGTGGCCTTCCACGGT | SEQ ID NO: 1243 |
| 213639_s_at | GCACATCCACGGTGGGTGAGTGGCC | SEQ ID NO: 1244 |
| 213639_s_at | GTCCTTGGTGGGTTTAGTCATCTCG | SEQ ID NO: 1245 |
| 213639_s_at | GTCATCTCGGAAGTCGTAGGGCAGC | SEQ ID NO: 1246 |
| 213639_s_at | GAACGTTCCAGCCAGGCAGTGGTTG | SEQ ID NO: 1247 |
| 213639_s_at | AGGCAGTGGTTGTTCCTCATAGGTA | SEQ ID NO: 1248 |
| 213639_s_at | TAGGTAGGTGGCCTTGGCCTTCATC | SEQ ID NO: 1249 |
| 213639_s_at | TCCATTGCATTTGTCACCTAGTCAC | SEQ ID NO: 1250 |
| 213639_s_at | GTATATACGTGCACATTTGACCTTT | SEQ ID NO: 1251 |
| 213639_s_at | TTCTCATTCCCTTAACTGACATTAT | SEQ ID NO: 1252 |
| 213639_s_at | TTTAGTGTCAGAGGCCGAGCACAGT | SEQ ID NO: 1253 |
| 214738_s_at | TTAGATTCAGATTCCTGGTGCCTCC | SEQ ID NO: 1254 |
| 214738_s_at | CCTGGGAACAGACTCCTGTAGACCC | SEQ ID NO: 1255 |
| 214738_s_at | CTAGTCTCCTGAGCCTATAGAGCCC | SEQ ID NO: 1256 |
| 214738_s_at | TAGAGCCCCCAGGAGACTGGGACCC | SEQ ID NO: 1257 |
| 214738_s_at | GGGACCCAAAGAACTTCACAGCACA | SEQ ID NO: 1258 |
| 214738_s_at | TCACAGCACACTTACCGAATGCAGA | SEQ ID NO: 1259 |
| 214738_s_at | TGCAGAGAGCAGCTTTCCTGGCTTT | SEQ ID NO: 1260 |
| 214738_s_at | GCAGAGGCTCTGAAGCACTTTCCTT | SEQ ID NO: 1261 |
| 214738_s_at | TAGCAACAGCAGCTCTGTACCTCAT | SEQ ID NO: 1262 |
| 214738_s_at | TCTGTTGATCCCACCTTTGAAGAGG | SEQ ID NO: 1263 |
| 214738_s_at | GACACAGTGCTCACCTTAATTGCGC | SEQ ID NO: 1264 |
| 214820_at | GATTTGGTTTCATCAGAAGCAGCAA | SEQ ID NO: 1265 |
| 214820_at | TTTTTGGTTATGGTGCTATTCCTAA | SEQ ID NO: 1266 |
| 214820_at | GGTGCTATTCCTAAGGTTAACTTTG | SEQ ID NO: 1267 |
| 214820_at | TAACTTTGAATATGTGACACACACA | SEQ ID NO: 1268 |
| 214820_at | CACACACACTCCTAAGTACCTTTAA | SEQ ID NO: 1269 |
| 214820_at | ATATTTTGACAGTTTAGGCTTCATT | SEQ ID NO: 1270 |
| 214820_at | GAACTATTCTGATTATTTGGACTGC | SEQ ID NO: 1271 |
| 214820_at | TGGACTGCATTAATTGGTCACTGAC | SEQ ID NO: 1272 |
| 214820_at | TAATTGGTCACTGACTGGCCATCCA | SEQ ID NO: 1273 |
| 214820_at | CTGGCCATCCAATTACCATTTTTTC | SEQ ID NO: 1274 |
| 214820_at | AATAGTTAGACCCTTGCATACAGAA | SEQ ID NO: 1275 |
| 219232_s_at | AAGCTTCTACTCCTGCAGTAAGCAC | SEQ ID NO: 1276 |
| 219232_s_at | CAGTAAGCACAGATCGCACTGCCTC | SEQ ID NO: 1277 |
| 219232_s_at | GATCGCACTGCCTCAATAACTTGGT | SEQ ID NO: 1278 |
| 219232_s_at | AACTTGGTATTGAGCACGTATTTTG | SEQ ID NO: 1279 |
| 219232_s_at | AATTTCCAGATAAGACATGTCACCA | SEQ ID NO: 1280 |
| 219232_s_at | CACCATTAATTCTCAACGACTGCTC | SEQ ID NO: 1281 |
| 219232_s_at | ACGACTGCTCTATTTTGTTGTACGG | SEQ ID NO: 1282 |
| 219232_s_at | GTACGGTAATAGTTATCACCTTCTA | SEQ ID NO: 1283 |
| 219232_s_at | TGTTTATTGTCTTGTATCCTTTCTC | SEQ ID NO: 1284 |
| 219232_s_at | GTATCCTTTCTCTGGAGTGTAAGCA | SEQ ID NO: 1285 |
| 219232_s_at | AATGCAACATACTCTCAGCACCTAA | SEQ ID NO: 1286 |
| 219383_at | TGTGCTCTTGATGGCTGGGAATTTA | SEQ ID NO: 1287 |
| 219383_at | TCTGCTGATCTGCTGAGAATTTCAA | SEQ ID NO: 1288 |
| 219383_at | TCTACAGACTGACTAACATGCATTA | SEQ ID NO: 1289 |
| 219383_at | GTAACTGATAGCTTCTGTCCTTATT | SEQ ID NO: 1290 |
| 219383_at | GCTTCTGTCCTTATTAGTACACTTA | SEQ ID NO: 1291 |
| 219383_at | GAGACTAGTATTTATTGATCCAGGC | SEQ ID NO: 1292 |
| 219383_at | CATGCTTGGGCTTACTTTTTCAGTT | SEQ ID NO: 1293 |
| 219383_at | AATGCCTTTTCCATATCTTAAATGT | SEQ ID NO: 1294 |
| 219383_at | ATAGACCCATTGTACTTAAGTGCTG | SEQ ID NO: 1295 |
| 219383_at | TAAGTGCTGATGACTGTTAGCCAGT | SEQ ID NO: 1296 |
| 219383_at | AGTTTACAACTTTTTACCATCGATG | SEQ ID NO: 1297 |
| 219718_at | GGTCACCGGATTGAAACTGTCTCAG | SEQ ID NO: 1298 |
| 219718_at | GTCTCAGGACCTTGATGATCTTGCC | SEQ ID NO: 1299 |
| 219718_at | TCTCTACCTGGCCACAGTTCAAGCC | SEQ ID NO: 1300 |
| 219718_at | CTTTGGGGACTCGCTTCATTATAGA | SEQ ID NO: 1301 |
| 219718_at | AGCAGGGCACTCAATCAGTACTCTT | SEQ ID NO: 1302 |
| 219718_at | TCTTTTCCTATGTGGAGGCCTCAGC | SEQ ID NO: 1303 |
| 219718_at | GCGGACATTACTGGCATGCCTGTGG | SEQ ID NO: 1304 |
| 219718_at | AGAGGTGGAGTCCGTTCTTGTGGGT | SEQ ID NO: 1305 |
| 219718_at | CCTCAGGGGATTTCGCTTCTGTACA | SEQ ID NO: 1306 |
| 219718_at | GAAAGTTGTGTTCCCGAGACTACAG | SEQ ID NO: 1307 |
| 219718_at | CAGGGCTTGCAGGTGCTGATGCCAG | SEQ ID NO: 1308 |
| 220360_at | TCAGAAAGTCTGTGTCGGGTCATAA | SEQ ID NO: 1309 |
| 220360_at | GAGCGAGTTGTAAGAACCCATTCAA | SEQ ID NO: 1310 |
| 220360_at | ATGGCAATTTTTGAACTAGTTTCTA | SEQ ID NO: 1311 |
| 220360_at | GAGCTTTCTGGGCATATTGATCTTT | SEQ ID NO: 1312 |
| 220360_at | GTGTGTGCCATCAATCACTTTGTCA | SEQ ID NO: 1313 |
| 220360_at | AAATGTTGCACAGAATCCTTTAAAA | SEQ ID NO: 1314 |
| 220360_at | GAAACACTGGTCATCTGTACAGGAT | SEQ ID NO: 1315 |
| 220360_at | ATGTTCAAGTTTTGCTAATACCAGT | SEQ ID NO: 1316 |
| 220360_at | TCAGGCATTTGCTAAGTAACGATGG | SEQ ID NO: 1317 |
| 220360_at | TTTGAAGTTCAATTTACCATATTTT | SEQ ID NO: 1318 |
| 220360_at | TAAATTGCGCATTCTGCACAGTGAA | SEQ ID NO: 1319 |
| 221020_s_at | CAGTTGGGATGCACTACCTAGCGAA | SEQ ID NO: 1320 |
| 221020_s_at | ACATCTATTGTCATTCCATTGCTAT | SEQ ID NO: 1321 |
| 221020_s_at | TAAAATCCTAGATCCAGTTCTTGTT | SEQ ID NO: 1322 |
| 221020_s_at | AAAATCTGAGCTTCTAGGATCCAGC | SEQ ID NO: 1323 |
| 221020_s_at | CTTCTAGGATCCAGCTGTGTCAACC | SEQ ID NO: 1324 |
| 221020_s_at | CTGTGTCAACCTTTATTTAGCATAT | SEQ ID NO: 1325 |
| 221020_s_at | ATAGATCACCTTTTACAGATGCTGA | SEQ ID NO: 1326 |
| 221020_s_at | GATTAATCTTCATTGGTTTCTCAAA | SEQ ID NO: 1327 |
| 221020_s_at | TAAAAGGGCCTGTACCCAAAGGATG | SEQ ID NO: 1328 |
| 221020_s_at | AAACATCCACGAGTGCTGTTGCACT | SEQ ID NO: 1329 |
| 221020_s_at | CTGTTGCACTACCATCTATTTGTTG | SEQ ID NO: 1330 |
| 221294_at | GCCAACGACCCTTACACAGTTAGAA | SEQ ID NO: 1331 |
| 221294_at | TCCTGATTTGGCTATACTCGACCCT | SEQ ID NO: 1332 |
| 221294_at | TTCAGTGGTGTGCGGAGTCCTGGCA | SEQ ID NO: 1333 |
| 221294_at | CTACTTCACCCTGTTCATCGTGATG | SEQ ID NO: 1334 |
| 221294_at | TGATGATGTTATATGCCCCAGCAGC | SEQ ID NO: 1335 |
| 221294_at | GGCCTGTCCTGATAAGCGCTATGCC | SEQ ID NO: 1336 |
| 221294_at | CTATGCCATGGTCCTGTTTCGAATC | SEQ ID NO: 1337 |
| 221294_at | GTATTTTACATCCTCTGGTTGCCAT | SEQ ID NO: 1338 |
| 221294_at | GGTTGCCATATATCATCTACTTCTT | SEQ ID NO: 1339 |
| 221294_at | GACTAAAGCGCCTCTCAGGGGCTAT | SEQ ID NO: 1340 |
| 221294_at | CAGGGGCTATGTGTACTTCTTGTGC | SEQ ID NO: 1341 |
| 34726_at | TGGCAGCCACATCCAAGACTGGAGC | SEQ ID NO: 1342 |
| 34726_at | CCAAGACTGGAGCAGCAGGCTGGCC | SEQ ID NO: 1343 |
| 34726_at | AGAGAGAGCTCACAGCTGAAGCTCT | SEQ ID NO: 1344 |
| 34726_at | AGCTCACAGCTGAAGCTCTTGGAGG | SEQ ID NO: 1345 |
| 34726_at | GACCAGGAGCATGGTGAAGCCAAGT | SEQ ID NO: 1346 |
| 34726_at | CCAAGTGGCAGATGGGAGCCAACCT | SEQ ID NO: 1347 |
| 34726_at | TTTGCCCTGCATCCTGTCATTTCTG | SEQ ID NO: 1348 |
| 34726_at | GTTCTTGTCCCTCATACATCTTTGG | SEQ ID NO: 1349 |
| 34726_at | TTGTCCCTCATACATCTTTGGAGAA | SEQ ID NO: 1350 |
| 34726_at | CCCTCATACATCTTTGGAGAACCGG | SEQ ID NO: 1351 |
| 34726_at | TCATACATCTTTGGAGAACCGGGCT | SEQ ID NO: 1352 |
| 34726_at | TGCCTTATGGCTCTAGTGTGTGACC | SEQ ID NO: 1353 |
| 34726_at | CTTATGGCTCTAGTGTGTGACCTAC | SEQ ID NO: 1354 |
| 34726_at | ATGGCTCTAGTGTGTGACCTACAGA | SEQ ID NO: 1355 |
| 34726_at | CTCTAGTGTGTGACCTACAGAGCAT | SEQ ID NO: 1356 |
| 34726_at | TGTGACCTACAGAGCATGCTCCACA | SEQ ID NO: 1357 |
| 34408_at | TCCGAGCTAAAATCCCAGGGACCGG | SEQ ID NO: 1358 |
| 34408_at | TTACCTGAGCGACCAGGACTACATT | SEQ ID NO: 1359 |
| 34408_at | GCCTGCTGGGACTTGTAGTTGCCTA | SEQ ID NO: 1360 |
| 34408_at | TGCTGGGACTTGTAGTTGCCTAGAC | SEQ ID NO: 1361 |
| 34408_at | TGGGACTTGTAGTTGCCTAGACAGG | SEQ ID NO: 1362 |
| 34408_at | TGTAGTTGCCTAGACAGGGCACCAC | SEQ ID NO: 1363 |
| 34408_at | GTAGTTGCCTAGACAGGGCACCACC | SEQ ID NO: 1364 |
| 34408_at | AGGCGTTGGTGTCTCCTGGATGCTA | SEQ ID NO: 1365 |
| 34408_at | GGCGTTGGTGTCTCCTGGATGCTAC | SEQ ID NO: 1366 |
| 34408_at | GCGTTGGTGTCTCCTGGATGCTACT | SEQ ID NO: 1367 |
| 34408_at | CGTTGGTGTCTCCTGGATGCTACTA | SEQ ID NO: 1368 |
| 34408_at | GGGAGGCCTGAGCTTGGATTTACAC | SEQ ID NO: 1369 |
| 34408_at | GGAGGCCTGAGCTTGGATTTACACT | SEQ ID NO: 1370 |
| 34408_at | GGCCTGAGCTTGGATTTACACTGTA | SEQ ID NO: 1371 |
| 34408_at | CTGAGCTTGGATTTACACTGTAATA | SEQ ID NO: 1372 |
| 34408_at | CTTGGATTTACACTGTAATAAAGAC | SEQ ID NO: 1373 |

TABLE 19
CE-HSC/LSC signature genes

| | | | Entrez | Representative |
|---|---|---|---|---|
| Probe Set ID | Gene Symbol | Gene Title | Gene ID | Public ID |
| 200672_x_at | SPTBN1 | spectrin, beta, non-erythrocytic 1 | 6711 | NM_003128 |
| 201917_s_at | SLC25A36 | solute carrier family 25, member 36 | 55186 | AI694452 |
| 201952_at | ALCAM | activated leukocyte cell adhesion molecule | 214 | AA156721 |
| 202932_at | YES1 | v-yes-1 Yamaguchi sarcoma viral oncogene homolog 1 | 7525 | NM_005433 |
| 203139_at | DAPK1 | death-associated protein kinase 1 | 1612 | NM_004938 |
| 203372_s_at | SOCS2 | suppressor of cytokine signaling 2 | 8835 | AB004903 |
| 203875_at | SMARCA1 | SWI/SNF related, matrix associated, actin dependent | 6594 | NM_003069 |
| | | regulator of chromatin, subfamily a, member 1 | | |
| 204069_at | MEIS1 | Meis homeobox 1 | 4211 | NM_002398 |
| 204753_s_at | HLF | hepatic leukemia factor | 3131 | AI810712 |
| 204754_at | HLF | hepatic leukemia factor | 3131 | W60800 |
| 204755_x_at | HLF | hepatic leukemia factor | 3131 | M95585 |
| 205376_at | INPP4B | inositol polyphosphate-4-phosphatase, type II, 105 kDa | 8821 | NM_003866 |
| 205453_at | HOXB2 | homeobox B2 | 3212 | NM_002145 |
| 205984_at | CRHBP | corticotropin releasing hormone binding protein | 1393 | NM_001882 |
| 206099_at | PRKCH | protein kinase C, eta | 5583 | NM_006255 |
| 206310_at | SPINK2 | serine peptidase inhibitor, Kazal type 2 (acrosin-trypsin | 6691 | NM_021114 |
| | | inhibitor) | | |
| 206478_at | KIAA0125 | KIAA0125 | 9834 | NM_014792 |
| 206674_at | FLT3 | fms-related tyrosine kinase 3 | 2322 | NM_004119 |
| 206683_at | ZNF165 | zinc finger protein 165 | 7718 | NM_003447 |
| 209487_at | RBPMS | RNA binding protein with multiple splicing | 11030 | D84109 |
| 209676_at | TFPI | tissue factor pathway inhibitor (lipoprotein-associated | 7035 | J03225 |
| | | coagulation inhibitor) | | |
| 209728_at | HLA-DRB4 | major histocompatibility complex, class II, DR beta 4 | 3126 | BC005312 |
| 209993_at | ABCB1 | ATP-binding cassette, sub-family B (MDR/TAP), member 1 | 5243 | AF016535 |
| 209994_s_at | ABCB1 /// | ATP-binding cassette, sub-family B (MDR/TAP), member 1 /// | 5243 /// | AF016535 |
| | ABCB4 | ATP-binding cassette, sub-family B (MDR/TAP), member 4 | 5244 | |
| 210664_s_at | TFPI | tissue factor pathway inhibitor (lipoprotein-associated | 7035 | AF021834 |
| | | coagulation inhibitor) | | |
| 210665_at | TFPI | tissue factor pathway inhibitor (lipoprotein-associated | 7035 | AF021834 |
| | | coagulation inhibitor) | | |
| 212071_s_at | SPTBN1 | spectrin, beta, non-erythrocytic 1 | 6711 | BE968833 |
| 212750_at | PPP1R16B | protein phosphatase 1, regulatory (inhibitor) subunit 16B | 26051 | AB020630 |
| 213056_at | FRMD4B | FERM domain containing 4B | 23150 | AU145019 |
| 213094_at | GPR126 | G protein-coupled receptor 126 | 57211 | AL033377 |
| 213541_s_at | ERG | v-ets erythroblastosis virus E26 oncogene homolog (avian) | 2078 | AI351043 |
| 213714_at | CACNB2 | calcium channel, voltage-dependent, beta 2 subunit | 783 | AI040163 |
| 213844_at | HOXA5 | homeobox A5 | 3202 | NM_019102 |
| 215388_s_at | CFH /// | complement factor H /// complement factor H-related 1 | 3075 /// | X56210 |
| | CFHR1 | | 3078 | |
| 217975_at | WBP5 | WW domain binding protein 5 | 51186 | NM_016303 |
| 218627_at | DRAM1 | DNA-damage regulated autophagy modulator 1 | 55332 | NM_018370 |
| 218764_at | PRKCH | protein kinase C, eta | 5583 | NM_024064 |
| 218772_x_at | TMEM38B | transmembrane protein 38B | 55151 | NM_018112 |
| 218899_s_at | BAALC | brain and acute leukemia, cytoplasmic | 79870 | NM_024812 |
| 218901_at | PLSCR4 | phospholipid scramblase 4 | 57088 | NM_020353 |
| 218966_at | MYO5C | myosin VC | 55930 | NM_018728 |
| 219497_s_at | BCL11A | B-cell CLL/lymphoma 11A (zinc finger protein) | 53335 | NM_022893 |
| 221458_at | HTR1F | 5-hydroxytryptamine (serotonin) receptor 1F | 3355 | NM_000866 |
| 221773_at | ELK3 | ELK3, ETS-domain protein (SRF accessory protein 2) | 2004 | AW575374 |
| 221942_s_at | GUCY1A3 | guanylate cyclase 1, soluble, alpha 3 | 2982 | AI719730 |
| 41577_at | PPP1R16B | protein phosphatase 1, regulatory (inhibitor) subunit 16B | 26051 | AB020630 |
| 222735_at | TMEM38B | transmembrane protein 38B | 55151 | AW452608 |
| 226547_at | MYST3 | MYST histone acetyltransferase (monocytic leukemia) 3 | 7994 | AI817830 |
| 228904_at | HOXB3 | homeobox B3 | 3213 | AW510657 |
| 235199_at | RNF125 | ring finger protein 125 | 54941 | AI969697 |
| 226420_at | MECOM | MDS1 and EVI1 complex locus | 2122 | BG261252 |

TABLE 20
The 19 HSC genes validated by qRT-PCR.

| Gene Symbol | Gene Title |
|---|---|
| ANK3 | Ankyrin 3, node of Ranvier (ankyrin G) |
| CRHBP | corticotropin releasing hormone binding protein |
| DUSP6 | dual specificity phosphatase 6 |
| EVI1 (or MECOM) | MDS1 and EVI1 complex locus |
| DRAM1 | DNA-damage regulated autophagy modulator 1 |
| KLF4 | Kruppel-like factor 4 (gut) |
| PROM1 | Prominin 1 |
| TFPI | tissue factor pathway inhibitor (lipoprotein- |
| | associated coagulation inhibitor) |
| ZNF165 | zinc finger protein 165 |
| ABCB1 | ATP-binding cassette, sub-family B |
| | (MDR/TAP), member 1 |
| BAALC | brain and acute leukemia, cytoplasmic |
| FLT3 | Fms-related tyrosine kinase 3 |
| FOXO1 | Forkhead box O1 |
| HLF | hepatic leukemia factor |
| HOXA5 | homeobox A5 |
| TMEM200A | transmembrane protein 200A |
| MEIS1 | Meis homeobox 1 |
| SOCS2 | suppressor of cytokine signaling 2 |
| DLK1 | delta-like 1 homolog (Drosophila) |

### CITATIONS FOR REFERENCES REFERRED TO IN THE BACKGROUND, SUMMARY, DETAILED DESCRIPTION AND EXAMPLE 1

1 Tallman, M S. New strategies for the treatment of acute myeloid leukemia including antibodies and other novel agents. Hematology. (Am. Soc. Hematol. Educ. Program.). 2005; 143-50:143-150
2 Appelbaum, F R, Rowe, J M, Radich, J, & Dick, J E. Acute myeloid leukemia. Hematology. Am. Soc. Hematol. Educ. Program. 2001; 62-86:62-86
3 Lapidot, T, Sirard, C, Vormoor, J, Murdoch, B, Hoang, T, Caceres-Cortes, J, Minden, M, Paterson, B, Caligiuri, M A, & Dick, J E. A cell initiating human acute myeloid leukaemia after transplantation into SCID mice. Nature. 1994; 367:645-648
4 Singh, S K, Clarke, I D, Terasaki, M, Bonn, V E, Hawkins, C, Squire, J, & Dirks, P B. Identification of a cancer stem cell in human brain tumors. Cancer Res. 2003; 63:5821-5828
5 Al Hajj, M, Wicha, M S, Benito-Hernandez, A, Morrison, S J, & Clarke, M F. Prospective identification of tumorigenic breast cancer cells. Proc. Natl. Acad. Sci. U.S.A. 2003; 100:3983-3988
6 Hemmati, H D, Nakano, I, Lazareff, J A, Masterman-Smith, M, Geschwind, D H, Bronner-Fraser, M, & Kornblum, H I. Cancerous stem cells can arise from pediatric brain tumors. Proc. Natl. Acad. Sci. U.S.A. 2003; 100:15178-15183
7 Yilmaz, O H, Valdez, R, Theisen, B K, Guo, W, Ferguson, D O, Wu, H, & Morrison, S J. Pten dependence distinguishes haematopoietic stem cells from leukaemia-initiating cells. Nature. 2006; 441:475-482
8 Zhang, J, Grindley, J C, Yin, T, Jayasinghe, S, He, X C, Ross, J T, Haug, J S, Rupp, D, Porter-Westpfahl, K S, Wiedemann, L M, Wu, H, & Li, L. PTEN maintains haematopoietic stem cells and acts in lineage choice and leukaemia prevention. Nature. 2006; 441:518-522
9 Gal, H, Amariglio, N, Trakhtenbrot, L, Jacob-Hirsh, J, Margalit, O, Avigdor, A, Nagler, A, Tavor, S, Ein-Dor, L, Lapidot, T, Domany, E, Rechavi, G et al. Gene expression profiles of AML derived stem cells; similarity to hematopoietic stem cells. Leukemia. 2006; 20:2147-2154
Majeti, R, Becker, M W, Tian, Q, Lee, T L, Yan, X, Liu, R, Chiang, J H, Hood, L, Clarke, M F, & Weissman, I L. Dysregulated gene expression networks in human acute myelogenous leukemia stem cells. Proc. Natl. Acad. Sci. U.S.A. 2009; 106:3396-3401
11 Grimwade, D, Walker, H, Oliver, F, Wheatley, K, Harrison, C, Harrison, G, Rees, J, Hann, I, Stevens, R, Burnett, A, & Goldstone, A. The importance of diagnostic cytogenetics on outcome in AML: analysis of 1,612 patients entered into the MRC AML 10 trial. The Medical Research Council Adult and Children's Leukaemia Working Parties. Blood. 1998; 92:2322-2333
12 Grimwade, D, Walker, H, Harrison, G, Oliver, F, Chatters, S, Harrison, C J, Wheatley, K, Burnett, A K, & Goldstone, A H. The predictive value of hierarchical cytogenetic classification in older adults with acute myeloid leukemia (AML): analysis of 1065 patients entered into the United Kingdom Medical Research Council AML11 trial. Blood. 2001; 98:1312-1320
13 Schlenk, R F, Dohner, K, Krauter, J, Frohling, S, Corbacioglu, A, Bullinger, L, Habdank, M, Spath, D, Morgan, M, Benner, A, Schlegelberger, B, Heil, G et al. Mutations and treatment outcome in cytogenetically normal acute myeloid leukemia. N. Engl. J. Med. 2008; 358:1909-1918
14 Langer, C, Marcucci, G, Holland, K B, Radmacher, M D, Maharry, K, Paschka, P, Whitman, S P, Mrozek, K, Baldus, C D, Vij, R, Powell, B L, Carroll, A J et al. Prognostic Importance of MN1 Transcript Levels, and Biologic Insights From MN1-Associated Gene and MicroRNA Expression Signatures in Cytogenetically Normal Acute Myeloid Leukemia: A Cancer and Leukemia Group B Study. J. Clin. Oncol. 2009;
15 Bullinger, L, Dohner, K, Bair, E, Frohling, S, Schlenk, R F, Tibshirani, R, Dohner, H, & Pollack, J R. Use of gene-expression profiling to identify prognostic subclasses in adult acute myeloid leukemia. N. Engl. J. Med. 2004; 350:1605-1616
16 Radmacher, M D, Marcucci, G, Ruppert, A S, Mrozek, K, Whitman, S P, Vardiman, J W, Paschka, P, Vukosavljevic, T, Baldus, C D, Kolitz, J E, Caligiuri, M A, Larson, R A et al. Independent confirmation of a prognostic gene-expression signature in adult acute myeloid leukemia with a normal karyotype: a Cancer and Leukemia Group B study. Blood. 2006; 108:1677-1683
17 Metzeler, K H, Hummel, M, Bloomfield, C D, Spiekermann, K, Braess, J, Sauerland, M C, Heinecke, A, Radmacher, M, Marcucci, G, Whitman, S P, Maharry, K, Paschka, P et al., An 86-probe-set gene-expression signature predicts survival in cytogenetically normal acute myeloid leukemia. Blood. 2008; 112:4193-4201
18 Smyth G K, Linear models and empirical bayes methods for assessing differential expression in microarray experiments. Stat Appl Genet Mol Biol. 2004; 3: Article 3
19 Schlenk, R. F. et al. Mutations and treatment outcome in cytogenetically normal acute myeloid leukemia. N. Engl. J. Med. 358, 1909-1918 (2008).
Dohner, K. et al. Mutant nucleophosmin (NPM1) predicts favorable prognosis in younger adults with acute myeloid leukemia and normal cytogenetics: interaction with other gene mutations. Blood 106, 3740-3746 (2005).
21 Schnittger, S. et al. Nucleophosmin gene mutations are predictors of favorable prognosis in acute myelogenous leukemia with a normal karyotype. Blood 106, 3733-3739 (2005).

### CITATIONS FOR REFERENCES REFERRED TO IN EXAMPLE 2

1. Dick, J. E. Stem cell concepts renew cancer research. Blood 112, 4793-4807 (2008).
2. Bao, S. et al. Glioma stem cells promote radioresistance by preferential activation of the DNA damage response. Nature 444, 756-760 (2006).
3. Diehn, M. et al. Association of reactive oxygen species levels and radioresistance in cancer stem cells. Nature 458, 780-783 (2009).
4. Li, X. et al. Intrinsic resistance of tumorigenic breast cancer cells to chemotherapy. J. Natl. Cancer Inst. 100, 672-679 (2008).
5. Ishikawa, F. et al. Chemotherapy-resistant human AML stem cells home to and engraft within the bone-marrow endosteal region. Nat. Biotechnol. 25, 1315-1321 (2007).
6. Dylla, S. J. et al. Colorectal cancer stem cells are enriched in xenogeneic tumors following chemotherapy. PLoS. One. 3, e2428 (2008).
7. Guzman, M. L. et al. Nuclear factor-kappaB is constitutively activated in primitive human acute myelogenous leukemia cells. Blood 98, 2301-2307 (2001).
8. Pearce, D. J. et al. AML engraftment in the NOD/SCID assay reflects the outcome of AML: implications for our understanding of the heterogeneity of AML. Blood 107, 1166-1173 (2006).
9. van Rhenen, A. et al. High stem cell frequency in acute myeloid leukemia at diagnosis predicts high minimal residual disease and poor survival. Clin. Cancer Res. 11, 6520-6527 (2005).
10. Wong, P., Iwasaki, M., Somervaille, T. C., So, C. W. & Cleary, M. L. Meis1 is an essential and rate-limiting regulator of MLL leukemia stem cell potential. Genes Dev. 21, 2762-2774 (2007).
11. Lessard, J. & Sauvageau, G. Bmi-1 determines the proliferative capacity of normal and leukaemic stem cells. Nature. 423, 255-260 (2003).
12. Liu, R. et al. The prognostic role of a gene signature from tumorigenic breast-cancer cells. N. Engl. J. Med. 356, 217-226 (2007).
13. Hussenet, T., Dembele, D., Martinet, N., Vignaud, J. M. & du, M. S. An adult tissue-specific stem cell molecular phenotype is activated in epithelial cancer stem cells and correlated to patient outcome. Cell Cycle 9, 321-327 (2010).
14. Massague, J. Sorting out breast-cancer gene signatures. N. Engl. J. Med. 356, 294-297 (2007).
15. Lapidot, T. et al. A cell initiating human acute myeloid leukaemia after transplantation into SCID mice. Nature. 367, 645-648 (1994).
16. Bonnet, D. & Dick, J. E. Human acute myeloid leukemia is organized as a hierarchy that originates from a primitive hematopoietic cell. Nat. Med. 3, 730-737 (1997).
17. Kelly, P. N., Dakic, A., Adams, J. M., Nutt, S. L. & Strasser, A. Tumor growth need not be driven by rare cancer stem cells. Science 317, 337 (2007).
18. Taussig, D. C. et al. Anti-CD38 antibody-mediated clearance of human repopulating cells masks the heterogeneity of leukemia-initiating cells. Blood 112, 568-575 (2008).
19. Taussig, D. C. et al. Leukemia-initiating cells from some acute myeloid leukemia patients with mutated nucleophosmin reside in the CD34(−) fraction. Blood 115, 1976-1984 (2010).
20. Quintana, E. et al. Efficient tumour formation by single human melanoma cells. Nature 456, 593-598 (2008).
21. Boiko, A. D. et al. Human melanoma-initiating cells express neural crest nerve growth factor receptor CD271. Nature 466, 133-137 (2010).
22. Schatton, T. et al. Identification of cells initiating human melanomas. Nature 451, 345-349 (2008).
23. McKenzie, J. L., Gan, O. I., Doedens, M. & Dick, J. E. Human short-term repopulating stem cells are efficiently detected following intrafemoral transplantation into NOD/SCID recipients depleted of CD122+ cells. Blood. 106, 1259-1261 (2005).
24. McDermott, S. P., Eppert, K., Lechman, E., Doedens, M. & Dick, J. E. Comparison of human cord blood engraftment between immunocompromised mouse strains. Blood (2010).
25. Georgantas, R. W., III et al. Microarray and serial analysis of gene expression analyses identify known and novel transcripts overexpressed in hematopoietic stem cells. Cancer Res. 64, 4434-4441 (2004).
26. Shojaei, F. et al., Hierarchical and ontogenic positions serve to define the molecular basis of human hematopoietic stem cell behavior. Dev. Cell 8, 651-663 (2005).
27. Wagner, W. et al. Molecular evidence for stem cell function of the slow-dividing fraction among human hematopoietic progenitor cells by genome-wide analysis. Blood. 104, 675-686 (2004).
28. Ivanova, N. B. et al. A stem cell molecular signature. Science. 298, 601-604 (2002).
29. Guzman, M. L. et al. Expression of tumor-suppressor genes interferon regulatory factor 1 and death-associated protein kinase in primitive acute myelogenous leukemia cells. Blood 97, 2177-2179 (2001).
30. Saito, Y. et al. Identification of therapeutic targets for quiescent, chemotherapy-resistant human leukemia stem cells. Sci. Transl. Med. 2, 17ra9 (2010).
31. Majeti, R. et al. Dysregulated gene expression networks in human acute myelogenous leukemia stem cells. Proc. Natl. Acad. Sci. U.S.A. 106, 3396-3401 (2009).
32. Gal, H. et al. Gene expression profiles of AML derived stem cells; similarity to hematopoietic stem cells. Leukemia. 20, 2147-2154 (2006).
33. Mazurier, F., Doedens, M., Gan, O. I. & Dick, J. E. Rapid myeloerythroid repopulation after intrafemoral transplantation of NOD-SCID mice reveals a new class of human stem cells. Nat. Med. 9, 959-963 (2003).
34. Subramanian, A. et al. Gene set enrichment analysis: a knowledge-based approach for interpreting genome-wide expression profiles. Proc. Natl. Acad. Sci. U.S. A 102, 15545-15550 (2005).
35. Mootha, V. K. et al. PGC-1alpha-responsive genes involved in oxidative phosphorylation are coordinately downregulated in human diabetes. Nat. Genet. 34, 267-273 (2003).
36. Brown, K. R. & Jurisica, I. Online predicted human interaction database. Bioinformatics. 21, 2076-2082 (2005).
37. Brown, K. R. et al. NAViGaTOR: Network Analysis, Visualization and Graphing Toronto. Bioinformatics. 25, 3327-3329 (2009).
38. Moore, M. A., Dorn, D. C., Schuringa, J. J., Chung, K. Y. & Morrone, G. Constitutive activation of Flt3 and STAT5A enhances self-renewal and alters differentiation of hematopoietic stem cells. Exp. Hematol. 35, 105-116 (2007).
39. Stier, S., Cheng, T., Dombkowski, D., Carlesso, N. & Scadden, D. T. Notch1 activation increases hematopoietic stem cell self-renewal in vivo and favors lymphoid over myeloid lineage outcome. Blood 99, 2369-2378 (2002).
40. Chung, Y. J. et al. Unique effects of Stat3 on the early phase of hematopoietic stem cell regeneration. Blood 108, 1208-1215 (2006).
41. Oh, I. H. & Eaves, C. J. Overexpression of a dominant negative form of STAT3 selectively impairs hematopoietic stem cell activity. Oncogene 21, 4778-4787 (2002).
42. Varnum-Finney, B. et al. Pluripotent, cytokine-dependent, hematopoietic stem cells are immortalized by constitutive Notch1 signaling. Nat. Med. 6, 1278-1281 (2000).
43. Karanu, F. N. et al. The notch ligand jagged-1 represents a novel growth factor of human hematopoietic stem cells. J. Exp. Med. 192, 1365-1372 (2000).
44. Ohishi, K., Varnum-Finney, B. & Bernstein, I. D. Delta-1 enhances marrow and thymus repopulating ability of human CD34(+)CD38(−) cord blood cells. J. Clin. Invest 110, 1165-1174 (2002).
45. Park, I. K. et al. Differential gene expression profiling of adult murine hematopoietic stem cells. Blood 99, 488-498 (2002).
46. Bhattacharya, B. et al. Gene expression in human embryonic stem cell lines: unique molecular signature. Blood 103, 2956-2964 (2004).
47. Ben Porath, I. et al. An embryonic stem cell-like gene expression signature in poorly differentiated aggressive human tumors. Nat. Genet. 40, 499-507 (2008).
48. Assou, S. et al. A meta-analysis of human embryonic stem cells transcriptome integrated into a web-based expression atlas. Stem Cells 25, 961-973 (2007).
49. Boyer, L. A. et al. Core transcriptional regulatory circuitry in human embryonic stem cells. Cell 122, 947-956 (2005).
50. Lee, T. I. et al. Control of developmental regulators by polycomb in human embryonic stem cells. Cell 125, 301-313 (2006).
51. Wong, D. J. et al. Module map of stem cell genes guides creation of epithelial cancer stem cells. Cell Stem Cell 2, 333-344 (2008).
52. Somervaille, T. C. et al. Hierarchical maintenance of MLL myeloid leukemia stem cells employs a transcriptional program shared with embryonic rather than adult stem cells. Cell Stem Cell 4, 129-140 (2009).
53. Valk, P. J. et al. Prognostically useful gene-expression profiles in acute myeloid leukemia. N. Engl. J. Med. 350, 1617-1628 (2004).
54. Verhaak, R. G. et al. Prediction of molecular subtypes in acute myeloid leukemia based on gene expression profiling. Haematologica 94, 131-134 (2009).
55. Metzeler, K. H. et al. An 86-probe-set gene-expression signature predicts survival in cytogenetically normal acute myeloid leukemia. Blood 112, 4193-4201 (2008).
56. Kottaridis, P. D. et al. The presence of a FLT3 internal tandem duplication in patients with acute myeloid leukemia (AML) adds important prognostic information to cytogenetic risk group and response to the first cycle of chemotherapy: analysis of 854 patients from the United Kingdom Medical Research Council AML 10 and 12 trials. Blood 98, 1752-1759 (2001).
57. Schlenk, R. F. et al. Mutations and treatment outcome in cytogenetically normal acute myeloid leukemia. N. Engl. J. Med. 358, 1909-1918 (2008).
58. Mrozek, K., Marcucci, G., Paschka, P., Whitman, S. P. & Bloomfield, C. D. Clinical relevance of mutations and gene-expression changes in adult acute myeloid leukemia with normal cytogenetics: are we ready for a prognostically prioritized molecular classification? Blood 109, 431-448 (2007).
59. Metzeler, K. H. et al. ERG expression is an independent prognostic factor and allows refined risk stratification in cytogenetically normal acute myeloid leukemia: a comprehensive analysis of ERG, MN1, and BAALC transcript levels using oligonucleotide microarrays. J. Clin. Oncol. 27, 5031-5038 (2009).
60. Dohner, K. et al. Mutant nucleophosmin (NPM1) predicts favorable prognosis in younger adults with acute myeloid leukemia and normal cytogenetics: interaction with other gene mutations. Blood 106, 3740-3746 (2005).
61. Schnittger, S. et al. Nucleophosmin gene mutations are predictors of favorable prognosis in acute myelogenous leukemia with a normal karyotype. Blood 106, 3733-3739 (2005).
62. Marcucci, G. et al. Prognostic significance of, and gene and microRNA expression signatures associated with, CEBPA mutations in cytogenetically normal acute myeloid leukemia with high-risk molecular features: a Cancer and Leukemia Group B Study. J. Clin. Oncol. 26, 5078-5087 (2008).
63. Rosen, J. M. & Jordan, C. T. The increasing complexity of the cancer stem cell paradigm. Science 324, 1670-1673 (2009).
64. Kennedy, J. A., Barabe, F., Poeppl, A. G., Wang, J. C. & Dick, J. E. Comment on “Tumor growth need not be driven by rare cancer stem cells”. Science 318, 1722 (2007).
65. Adams, J. M. & Strasser, A. Is tumor growth sustained by rare cancer stem cells or dominant clones? Cancer Res. 68, 4018-4021 (2008).
66. Shackleton, M., Quintana, E., Fearon, E. R. & Morrison, S. J. Heterogeneity in cancer: cancer stem cells versus clonal evolution. Cell 138, 822-829 (2009).
67. Goyama, S. et al. Evi-1 is a critical regulator for hematopoietic stem cells and transformed leukemic cells. Cell Stem Cell 3, 207-220 (2008).
68. Simsek, T. et al. The Distinct Metabolic Profile of Hematopoietic Stem Cells Reflects Their Location in a Hypoxic Niche. Cell Stem Cell 7, 380-390 (2010).
69. Bjornsson, J. M. et al. Reduced proliferative capacity of hematopoietic stem cells deficient in Hoxb3 and Hoxb4. Mol. Cell Biol. 23, 3872-3883 (2003).
70. Loughran, S. J. et al. The transcription factor Erg is essential for definitive hematopoiesis and the function of adult hematopoietic stem cells. Nat. Immunol. 9, 810-819 (2008).
71. Barjesteh van Waalwijk van Doorn-Khosrovani et al., High EVI1 expression predicts poor survival in acute myeloid leukemia: a study of 319 de novo AML patients. Blood 101, 837-845 (2003).
72. Krivtsov, A. V. et al. Transformation from committed progenitor to leukaemia stem cell initiated by MLL-AF9. Nature 442, 818-822 (2006).
73. Chen, W. et al. Malignant transformation initiated by MII-AF9: gene dosage and critical target cells. Cancer Cell 13, 432-440 (2008).
74. Huntly, B. J. et al. MOZ-TIF2, but not BCR-ABL, confers properties of leukemic stem cells to committed murine hematopoietic progenitors. Cancer Cell 6, 587-596 (2004).
75. Cozzio, A. et al. Similar MLL-associated leukemias arising from self-renewing stem cells and short-lived myeloid progenitors. Genes Dev. 17, 3029-3035 (2003).
76. Tenen, D. G. Disruption of differentiation in human cancer: AML shows the way. Nat. Rev. Cancer 3, 89-101 (2003).
77. Kroon, E. et al. Hoxa9 transforms primary bone marrow cells through specific collaboration with Meisla but not Pbxlb. EMBO J. 17, 3714-3725 (1998).
78. Baugh, L. R., Hill, A. A., Brown, E. L. & Hunter, C. P. Quantitative analysis of mRNA amplification by in vitro transcription. Nucleic Acids Res. 29, E29 (2001).
79. Saeed, A. I. et al. TM4 microarray software suite. Methods Enzymol. 411, 134-193 (2006).
80. Saeed, A. I. et al. TM4: a free, open-source system for microarray data management and analysis. BioTechniques 34, 374-378 (2003).
81. Huang, d. W., Sherman, B. T. & Lempicki, R. A. Systematic and integrative analysis of large gene lists using DAVID bioinformatics resources. Nat. Protoc. 4, 44-57 (2009).
82. Dennis, G., Jr. et al. DAVID: Database for Annotation, Visualization, and Integrated Discovery. Genome Biol. 4, 3 (2003).
83. Stark, C. et al. BioGRID: a general repository for interaction datasets. Nucleic Acids Res. 34, D535-D539 (2006).
84. Salwinski, L. et al. The Database of Interacting Proteins: 2004 update. Nucleic Acids Res. 32, D449-D451 (2004).
85. Keshava Prasad, T. S. et al. Human Protein Reference Database—2009 update. Nucleic Acids Res. 37, D767-D772 (2009).
86. Aranda, B. et al. The IntAct molecular interaction database in 2010. Nucleic Acids Res. 38, D525-D531 (2010).
87. Chatr-Aryamontri, A. et al. MINT: the Molecular INTeraction database. Nucleic Acids Res. 35, D572-D574 (2007).
88. McGuffin, M. J. & Jurisica, I. Interaction techniques for selecting and manipulating subgraphs in network visualizations. IEEE Trans. Vis. Comput. Graph. 15, 937-944 (2009).
89. Buchner, T. et al. Double induction containing either two courses or one course of high-dose cytarabine plus mitoxantrone and postremission therapy by either autologous stem-cell transplantation or by prolonged maintenance for acute myeloid leukemia. J. Clin. Oncol. 24, 2480-2489 (2006).
90. Hu, Y. & Smyth, G. K. ELDA: extreme limiting dilution analysis for comparing depleted and enriched populations in stem cell and other assays. J. Immunol. Methods 347, 70-78 (2009).
91. Smyth, G. K. Linear models and empirical bayes methods for assessing differential expression in microarray experiments. Stat. Appl. Genet. Mol. Biol. 3, Article 3 (2004).

### CITATIONS FOR REFERENCES REFERRED TO IN EXAMPLES 3 TO 6

1. Kartner, N., Evernden-Porelle, D., Bradley, G. & Ling, V. Detection of P-glycoprotein in multidrug-resistant cell lines by monoclonal antibodies. Nature 316, 820-823 (1985).
2. Riordan, J. R. et al. Amplification of P-glycoprotein genes in multidrug-resistant mammalian cell lines. Nature 316, 817-819 (1985).
3. Goodell, M. A., Brose, K., Paradis, G., Conner, A. S. & Mulligan, R. C. Isolation and functional properties of murine hematopoietic stem cells that are replicating in vivo. J. Exp. Med. 183, 1797-1806 (1996).
4. Bunting, K. D., Zhou, S., Lu, T. & Sorrentino, B. P. Enforced P-glycoprotein pump function in murine bone marrow cells results in expansion of side population stem cells in vitro and repopulating cells in vivo. Blood 96, 902-909 (2000).
5. Campos, L. et al. Clinical significance of multidrug resistance P-glycoprotein expression on acute nonlymphoblastic leukemia cells at diagnosis. Blood 79, 473-476 (1992).
6. Dalerba, P. et al. Phenotypic characterization of human colorectal cancer stem cells. Proc. Natl. Acad. Sci. U.S. A 104, 10158-10163 (2007).
7. Ofori-Acquah, S. F. & King, J. A. Activated leukocyte cell adhesion molecule: a new paradox in cancer. Transl. Res. 151, 122-128 (2008).
8. Kahlert, C. et al. Increased expression of ALCAM/CD166 in pancreatic cancer is an independent prognostic marker for poor survival and early tumour relapse. Br. J. Cancer 101, 457-464 (2009).
9. Tanner, S. M. et al. BAALC, the human member of a novel mammalian neuroectoderm gene lineage, is implicated in hematopoiesis and acute leukemia. Proc. Natl. Acad. Sci. U.S. A 98, 13901-13906 (2001).
10. Metzeler, K. H. et al. ERG expression is an independent prognostic factor and allows refined risk stratification in cytogenetically normal acute myeloid leukemia: a comprehensive analysis of ERG, MN1, and BAALC transcript levels using oligonucleotide microarrays. J. Clin. Oncol. 27, 5031-5038 (2009).
11. Baldus, C. D. et al. BAALC expression predicts clinical outcome of de novo acute myeloid leukemia patients with normal cytogenetics: a Cancer and Leukemia Group B Study. Blood 102, 1613-1618 (2003).
12. Baldus, C. D. et al. BAALC, a novel marker of human hematopoietic progenitor cells. Exp. Hematol. 31, 1051-1056 (2003).
13. Satterwhite, E. et al. The BCL11 gene family: involvement of BCL11A in lymphoid malignancies. Blood 98, 3413-3420 (2001).
14. Deiss, L. P., Feinstein, E., Berissi, H., Cohen, O. & Kimchi, A. Identification of a novel serine/threonine kinase and a novel 15-kD protein as potential mediators of the gamma interferon-induced cell death. Genes Dev. 9, 15-30 (1995).
15. Raval, A. et al. Downregulation of death-associated protein kinase 1 (DAPK1) in chronic lymphocytic leukemia. Cell 129, 879-890 (2007).
16. Loughran, S. J. et al. The transcription factor Erg is essential for definitive hematopoiesis and the function of adult hematopoietic stem cells. Nat. Immunol. 9, 810-819 (2008).
17. Shimizu, K. et al. An ets-related gene, ERG, is rearranged in human myeloid leukemia with t(16;21) chromosomal translocation. Proc. Natl. Acad. Sci. U.S. A 90, 10280-10284 (1993).
18. Sorensen, P. H. et al. A second Ewing's sarcoma translocation, t(21;22), fuses the EWS gene to another ETS-family transcription factor, ERG. Nat. Genet. 6, 146-151 (1994).
19. Marcucci, G. et al. Overexpression of the ETS-related gene, ERG, predicts a worse outcome in acute myeloid leukemia with normal karyotype: a Cancer and Leukemia Group B study. J. Clin. Oncol. 23, 9234-9242 (2005).
20. Baldus, C. D. et al. Acute myeloid leukemia with complex karyotypes and abnormal chromosome 21: Amplification discloses overexpression of APP, ETS2, and ERG genes. Proc. Natl. Acad. Sci. U.S. A 101, 3915-3920 (2004).
21. Goyama, S. et al. Evi-1 is a critical regulator for hematopoietic stem cells and transformed leukemic cells. Cell Stem Cell 3, 207-220 (2008).
22. Goyama, S. & Kurokawa, M. Pathogenetic significance of ecotropic viral integration site-1 in hematological malignancies. Cancer Sci. 100, 990-995 (2009).
23. Barjesteh van Waalwijk van Doorn-Khosrovani et al. High EVI1 expression predicts poor survival in acute myeloid leukemia: a study of 319 de novo AML patients. Blood 101, 837-845 (2003).
24. Moore, M. A., Dorn, D. C., Schuringa, J. J., Chung, K. Y. & Morrone, G. Constitutive activation of Flt3 and STAT5A enhances self-renewal and alters differentiation of hematopoietic stem cells. Exp. Hematol. 35, 105-116 (2007).
25. Christensen, J. L. & Weissman, I. L. Flk-2 is a marker in hematopoietic stem cell differentiation: a simple method to isolate long-term stem cells. Proc. Natl. Acad. Sci. U. S. A 98, 14541-14546 (2001).
26. Adolfsson, J. et al. Upregulation of Flt3 expression within the bone marrow Lin(−) Sca1(+)c-kit(+) stem cell compartment is accompanied by loss of self-renewal capacity. Immunity. 15, 659-669 (2001).
27. Kottaridis, P. D. et al. The presence of a FLT3 internal tandem duplication in patients with acute myeloid leukemia (AML) adds important prognostic information to cytogenetic risk group and response to the first cycle of chemotherapy: analysis of 854 patients from the United Kingdom Medical Research Council AML 10 and 12 trials. Blood 98, 1752-1759 (2001).
28. Schlenk, R. F. et al., Mutations and treatment outcome in cytogenetically normal acute myeloid leukemia. N. Engl. J. Med. 358, 1909-1918 (2008).
29. Mrozek, K., Marcucci, G., Paschka, P., Whitman, S. P. & Bloomfield, C. D. Clinical relevance of mutations and gene-expression changes in adult acute myeloid leukemia with normal cytogenetics: are we ready for a prognostically prioritized molecular classification? Blood 109, 431-448 (2007).
30. Dorak, M. T. et al. A male-specific increase in the HLA-DRB4 (DR53) frequency in high-risk and relapsed childhood ALL. Leuk. Res. 26, 651-656 (2002).
31. Inaba, T. et al. Fusion of the leucine zipper gene HLF to the E2A gene in human acute B-lineage leukemia. Science 257, 531-534 (1992).
32. Shojaei, F. et al., Hierarchical and ontogenic positions serve to define the molecular basis of human hematopoietic stem cell behavior. Dev. Cell 8, 651-663 (2005).
33. Strathdee, G., Sim, A., Soutar, R., Holyoake, T. L. & Brown, R. HOXA5 is targeted by cell-type-specific CpG island methylation in normal cells and during the development of acute myeloid leukaemia. Carcinogenesis 28, 299-309 (2007).
34. Strathdee, G. et al. Inactivation of HOXA genes by hypermethylation in myeloid and lymphoid malignancy is frequent and associated with poor prognosis. Clin. Cancer Res. 13, 5048-5055 (2007).
35. Mullighan, C. G. et al. Pediatric acute myeloid leukemia with NPM1 mutations is characterized by a gene expression profile with dysregulated HOX gene expression distinct from MLL-rearranged leukemias. Leukemia 21, 2000-2009 (2007).
36. Sauvageau, G. et al. Differential expression of homeobox genes in functionally distinct CD34+ subpopulations of human bone marrow cells. Proc. Natl. Acad. Sci. U.S. A 91, 12223-12227 (1994).
37. Bjornsson, J. M. et al. Reduced proliferative capacity of hematopoietic stem cells deficient in Hoxb3 and Hoxb4. Mol. Cell Biol. 23, 3872-3883 (2003).
38. Thorsteinsdottir, U., Kroon, E., Jerome, L., Blasi, F. & Sauvageau, G. Defining roles for HOX and MEIS1 genes in induction of acute myeloid leukemia. Mol. Cell Biol. 21, 224-234 (2001).
39. Gewinner, C. et al. Evidence that inositol polyphosphate 4-phosphatase type II is a tumor suppressor that inhibits PI3K signaling. Cancer Cell 16, 115-125 (2009).
40. Armstrong, S. A. et al. MLL translocations specify a distinct gene expression profile that distinguishes a unique leukemia. Nat. Genet. 30, 41-47 (2002).
41. Rozovskaia, T. et al. Upregulation of Meis1 and HoxA9 in acute lymphocytic leukemias with the t(4:11) abnormality. Oncogene 20, 874-878 (2001).
42. Kroon, E. et al. Hoxa9 transforms primary bone marrow cells through specific collaboration with Meis1a but not Pbx1b. EMBO J. 17, 3714-3725 (1998).
43. Pineault, N. et al. Induction of acute myeloid leukemia in mice by the human leukemia-specific fusion gene NUP98-HOXD13 in concert with Meis1. Blood 101, 4529-4538 (2003).
44. Wong, P., Iwasaki, M., Somervaille, T. C., So, C. W. & Cleary, M. L. Meis1 is an essential and rate-limiting regulator of MLL leukemia stem cell potential. Genes Dev. 21, 2762-2774 (2007).
45. Simsek, T. et al. The Distinct Metabolic Profile of Hematopoietic Stem Cells Reflects Their Location in a Hypoxic Niche. Cell Stem Cell 7, 380-390 (2010).
46. Yang, X. J. & Ullah, M. MOZ and MORF, two large MYSTic HATs in normal and cancer stem cells. Oncogene 26, 5408-5419 (2007).
47. Thomas, T. et al. Monocytic leukemia zinc finger protein is essential for the development of long-term reconstituting hematopoietic stem cells. Genes Dev. 20, 1175-1186 (2006).
48. Grand, F. H. et al. A constitutively active SPTBN1-FLT3 fusion in atypical chronic myeloid leukemia is sensitive to tyrosine kinase inhibitors and immunotherapy. Exp. Hematol. 35, 1723-1727 (2007).
49. Ramalho-Santos, M., Yoon, S., Matsuzaki, Y., Mulligan, R. C. & Melton, D. A. “Stemness”: transcriptional profiling of embryonic and adult stem cells. Science. 298, 597-600 (2002).
50. Anneren, C., Cowan, C. A. & Melton, D. A. The Src family of tyrosine kinases is important for embryonic stem cell self-renewal. J. Biol. Chem. 279, 31590-31598 (2004).
51. Seki, T., Fujii, G., Mori, S., Tamaoki, N. & Shibuya, M. Amplification of c-yes-1 proto-oncogene in a primary human gastric cancer. Jpn. J. Cancer Res. 76, 907-910 (1985).
52. Georgantas, R. W., III et al. Microarray and serial analysis of gene expression analyses identify known and novel transcripts overexpressed in hematopoietic stem cells. Cancer Res. 64, 4434-4441 (2004).
53. Park, I. K. et al. Differential gene expression profiling of adult murine hematopoietic stem cells. Blood 99, 488-498 (2002).
54. Ivanova, N. B. et al. A stem cell molecular signature. Science. 298, 601-604 (2002).
55. Majeti, R. et al. Dysregulated gene expression networks in human acute myelogenous leukemia stem cells. Proc. Natl. Acad. Sci. U.S.A. 106, 3396-3401 (2009).
56. Saito, Y. et al. Identification of therapeutic targets for quiescent, chemotherapy-resistant human leukemia stem cells. Sci. Transl. Med. 2, 17ra9 (2010).
57. Ishikawa, F. et al. Chemotherapy-resistant human AML stem cells home to and engraft within the bone-marrow endosteal region. Nat. Biotechnol. 25, 1315-1321 (2007).
58. Gal, H. et al. Gene expression profiles of AML derived stem cells; similarity to hematopoietic stem cells. Leukemia. 20, 2147-2154 (2006).

1. A method for determining a prognosis of a subject having leukemia or myelodysplastic syndrome (MDS) comprising:
a) obtaining a sample from a subject;
b) determining a gene expression level for each gene of a set of genes selected from leukemia stem cell (LSC) signature genes listed in Tables 2, 6, and/or 12, hematopoietic stem cell (HSC) signature genes listed in Tables 4 and/or 14, and/or CE-HSC/LSC signature genes listed in Table 19, to obtain a subject expression profile of a sample obtained from the subject; and
c) classifying the subject as having a good prognosis or a poor prognosis based on the subject expression profile;
wherein a good prognosis predicts an increased likelihood of survival within a predetermined period after initial diagnosis and poor prognosis predicts a decreased likelihood of survival within the predetermined period after initial diagnosis.

2. (canceled)

3. The method of claim 1, wherein the set of genes comprises at least two genes listed in Table 2 and/or 6, the genes listed in Table 4 and/or 14 and/or the genes listed in Table 19, optionally wherein the set of genes comprises ceroid lipofuscinosis neuronal 5 (CLN5) or neurofibromin 1 (NF1).

4.-9. (canceled)

10. The method of claim 1, wherein the subject expression profile is used to calculate a subject risk score, wherein the subject is classified as having a good prognosis if the subject risk score is low and/or below a selected threshold and as having a poor prognosis if the subject risk score is high and/or above the selected threshold.

11. A method for monitoring a response to a treatment in a subject having leukemia or myelodysplastic syndrome (MDS) comprising:
a. collecting a first sample from the subject before the subject has received the treatment;
b. collecting a subsequent sample from the subject after the subject has received the treatment;
c. determining the gene expression levels of a set of genes selected from LSC signature genes and/or HSC signature genes in the first and the subsequent samples according to the method of claim 1, to obtain a first sample subject expression profile and a subsequent sample subject expression profile, wherein the set of genes comprises at least 2 genes; and
d. calculating a first sample subject expression profile score and a subsequent sample subject expression profile score;
wherein a lower subsequent sample expression profile score compared to the first sample expression profile score is indicative of a positive response, and a higher subsequent sample expression profile score compared to the first expression profile score is indicative of a negative response.

12. The method of claim 10, wherein the subject expression profile score is calculated by:
a. calculating log 2 expression value of the set of genes for the sample;
b. centering the log 2 expression value of step a to a zero mean; and
c. taking the sum of the log 2 expression values to give the subject risk score.

13. (canceled)

14. The method of claim 1, wherein the gene expression level is determined by detecting mRNA expression using one or more probes and/or one or more probe sets, optionally wherein the one or more probes and/or the one or more probe sets are selected from SEQ ID NOs:1-2533.

15.-17. (canceled)

18. The method of claim 1, wherein the leukemia is AML, ALL, CML or CLL.

19. The method of claim 18 wherein the AML is cytogenetically normal AML (CN-AML).

20. (canceled)

21. The method of claim 1, further comprising the step of providing a cancer treatment to the subject suitable with the prognosis determined.

22. The method of claim 1, further comprising the classifying the subject as low molecular risk (LMR) or high molecular risk (HMR) according to Nucleophosmin (NPM1) and FLT3 mutated internal tandem duplication (FLT3ITD) status, wherein the subject is identified as LMR if the subject comprises a mutant NPMI gene and is FLT3IT positive, and is identified as HMR if the subject has a wildtype NPMI gene and is FLT3ITD negative.

23.-26. (canceled)

27. The method of claim 1, wherein the gene expression level is determined using Nanostring® technology, serial analysis of gene expression (SAGE), RNA sequencing, RNase protection assays, Northern Blot, a microarray chip and/or a PCR protocol, optionally multiplex PCR.

28. (canceled)

29. (canceled)

30. The method of claim 1, further comprising displaying or outputting a result of one of the steps to a user interface device, a computer readable storage medium, a monitor, or a computer that is part of a network.

31. A method of treating a subject having leukemia or myelodysplastic syndrome (MDS), comprising determining a prognosis of the subject according to the method of claim 1, and providing a suitable treatment to the subject in need thereof according to the prognosis determined.

32. The method of claim 31, wherein the subject is determined to have a poor prognosis, and the treatment comprises a stem cell transplant.

33. (canceled)

34. A composition comprising a set of nucleic acid molecules each comprising a polynucleotide probe sequence selected from SEQ ID NO:1-2533.

35.-37. (canceled)

38. An array comprising for each gene in a set of genes, the set of genes comprising at least 2 of the genes listed in Table 2, 4, 6, 12 and/or 14, one or more polynucleotide probes complementary and hybridizable to a coding sequence in the gene, for determining a prognosis according to claim 1.

39. (canceled)

40. The array of claim 38 wherein the one or more polynucleotide probes are selected from SEQ ID NO:1-2533.

41. A kit for determining prognosis in a subject having a hematological cancer according to the method of claim 1 comprising:
a) an array of claim 38 a set of probes wherein each probe of the set hybridizes and/or is complementary to a nucleic acid sequence corresponding to a gene selected from Table 2, 4, 6, 12 and/or 14 or one or more primers or sets of primers, each primer or set of primers specific for a gene selected from Table 2, 4, 6, 12 and/or 14;
b) a kit control; and
c) optionally instructions for use.

42. (canceled)

43. (canceled)

44. A non-transitory computer readable storage medium with an executable program stored thereon, wherein the program is for predicting outcome or prognosis in a subject having a hematological cancer, and wherein the program instructs a microprocessor to perform one or more of the steps of claim 1.

45. A computer system for performing one or more steps of claim 1 comprising:
a) a database including records comprising reference expression profiles associated with clinical outcomes, each reference profile comprising the expression levels of a set of genes listed in Table 2, 4, 6, 12 and/or 14;
b) a user interface capable of receiving and/or inputting a selection of gene expression levels of a set of genes, the set comprising at least 2 genes listed in Table 2, 4, 6, and/or 14 for use in comparing to the gene reference expression profiles in the database;
c) an output that displays a prediction of clinical prognosis according to the expression levels of the set of genes.

46. (canceled)
